(19) United States
(12) Patent Application Publication
(10) Pub. No.: US 2008/0076924 A1
(43) Pub. Date: Mar. 27, 2008

(54) Piperazines as P2X7 antagonists

(76) Inventors: Patrick Betschmann, Bueren SO (CH); William A. Carroll, Evanston, IL (US); Anna M. Ericsson, Shrewsbury, MA (US); Shannon R. Fix-Stenzel, Shrewsbury, MA (US); Michael Friedman, Newton, MA (US); Gavin C. Hirst, San Diego, CA (US); Nathan S. Josephsohn, Boston, MA (US); Biqin Li, Northborough, MA (US); Arturo Perez-Medrano, Grayslake, IL (US); Michael J. Morytko, Framingham, MA (US); Paul Rafferty, Westborough, MA (US); Haipeng Chen, Chesterfield, MO (US)

Correspondence Address: ABBOTT BIORESEARCH, 100 RESEARCH DRIVE, WORCESTER, MA, 01605-4314, US

(21) Appl. No.: 11/824,441

(22) Filed: Jun. 29, 2007

Related U.S. Application Data
(60) Provisional application No. 60/818,263, filed on Jun. 30, 2006.

(51) Int. Cl. C07D 471/04; C07D 401/00

(52) U.S. Cl. 544/279; 544/363; 544/382

(57) ABSTRACT

Novel compounds of Formula (I) or pharmaceutically acceptable salts thereof, metabolites thereof, isomers thereof, enantiomers thereof or prodrugs thereof of Formula (I) 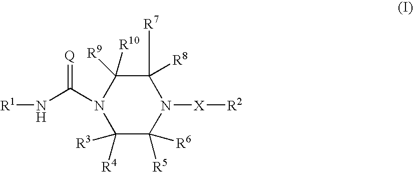 wherein the substituents are as defined herein, which are useful as therapeutic agents.

### CROSS REFERENCE TO PRIORITY APPLICATION

This application claims priority to U.S. provisional application 60/818,263 filed Jun. 30, 2006.

### BACKGROUND OF THE INVENTION

The P2X₇ purinergic receptor (previously known as P2Z), which is one of a family of ligand gated ion channels, is present on a variety of cell types known to be involved in inflammatory and immune processes, specifically macrophages, mast cells and (T and B) lymphocytes. Activation of the P2X₇ receptor by extracellular nucleotides, in particular ATP, leads to caspase-1 (ICE) activation, a protease required for the processing and release of IL-1b and IL-18. In addition, giant cell formation (macrophages), degranulation (mast cells), and proliferation (T-cells) apoptosis and L-selectin shedding (lymphocytes) occurs on P2X₇R activation. In addition in a mouse anti-collagen arthritis model, P2X₇ deficient animals showed a substantial reduction in symptom severity relative to wild-type controls.

The P2X₇R is also known to be a pain sensor in the nervous system is expressed by CNS microglia and peripheral nerves. Experiments using P2X₇ deficient mice demonstrate the role of P2X₇ in the development of pain as these mice were protected from the development of both adjuvant-induced inflammatory pain and partial nerve ligation induced neuropathic pain.

In view of the clinical importance of P2X₇, the identification of compounds that modulate P2X₇ receptor function represents an attractive avenue into the development of new therapeutic agents. Such compounds are provided herein.

### SUMMARY OF THE INVENTION

The present invention provides a compound of Formula I 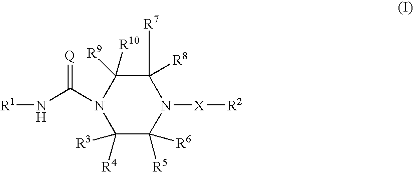
pharmaceutically acceptable salts thereof, metabolites thereof, isomers thereof, enantiomers thereof, or prodrugs thereof wherein
Q is N—CN or S;
X is selected from the group consisting of a bond, C(O), C(O)—N(X¹), S(O)₂ and C(O)—N(X¹)—S(O)₂;
   wherein X¹ is H or alkyl;
R¹ is selected from the optionally substituted group consisting of diphenylalkyl, alkoxy, alkoxycarbonylalkyl, alkyl, amino, aryl, arylalkyl, benzyloxy, cycloalkyl, cycloalkylalkyl, heteroaryl and heterocyclyl; or
R¹ is A-B wherein A is attached to the nitrogen of the amino and
   A is —C(O)— or is selected from the optionally substituted group consisting of alkylidenyl, heterocyclyl, aryl and heteroaryl;
   B is selected from the optionally substituted group consisting of —(CH₂), —C(O)—O(CH₂)ₙ-aryl, —(CH₂)ₙ—NR^{a}—C(O)—O(CH₂)ₙ-phenyl, —NR^{a}—C(O)—(CH₂)ₙ—NR^{a}R^{b}, —C(O)—O(CH₂)ₙ-aryl, C(O)—(CH₂)ₙ—C(O)—NR^{a}R^{b}, —C(O)—R^{c}—NR^{a}—C(O)-cycloalkyl, —NR^{a}—C(O)—(CH₂)_{c}ycloalkyl, —NR^{a}—C(O)—O(CH₂)ₙ-phenyl, alkoxy, alkyl-NR^{a}R^{b}, NR^{a}-alkyl-NR^{a}R^{b}, aryl, aryloxy, benzyloxy, cycloalkyl, cycloalkylalkyl, heterocyclyl, heterocyclyoalkyl, heterocycloalkylamine and heteroaryl; or
R² is H or —(CH₂)ₙ—C(O)O-alkyl; or
R² is selected from the optionally substituted group consisting of alkyl, aryl, arylalkyl, cycloalkyl, heteroaryl and heterocyclyl; or
R² is Y-Z wherein Y is attached to X and
   Y is selected from the group consisting of alkylidenyl, alkenyl, aryl, cycloalkenyl, heteroaryl, heterocyclyl and 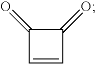
   Z is —NR^{a}(CH₂)ₙ-Z¹⁰⁰, —NR^{a}—(CH₂)ₙ—C(O)-Z¹⁰⁰, —NR^{a}(CH₂)ₙ—S(O)₂-Z²⁰⁰, —NR^{a}—C(O)—(CH₂), —OH or is selected from the optionally substituted group consisting of —(CH₂)ₙ—NR^{a}—C(O)—O(CH₂)ₙ-aryl, -alkylNR^{a}R^{b}, aryl, aryloxy, benzyloxy, heteroaryl, heterocyclyl, —C(O)NR^{a}(CH₂), OH, —C(O)—O(CH₂)ₙ-aryl, —C(O)—R^{d}, —C(O)—NR^{a}R^{b}, —C(O)—NR^{a}—R^{d}, —O—R^{d} and —NR^{a}—R^{d};
   wherein Z¹⁰⁰ is selected from the group consisting of OH, —NR^{a}R^{b}, alkoxy or optionally substituted heterocyclyl;
   wherein Z²⁰⁰ is selected from the group consisting of alkyl and optionally substituted heterocyclyl and optionally substituted heterocyclylalkyl;
R³, R⁴, R⁵, R⁶, R⁷, R⁸, R⁹ and R¹⁰ are independently H, COOH, —C(O)—NH₂, —CH₂—O—(CH₂)ₘ—O—CH₂CH₂—OCH₃, —CH₂—O—CH₂—O—(CH₂)ₘ—OCH₃ or are independently selected from the optionally substituted group consisting of alkyl, alkenyl, alkynyl, alkoxyalkyl, cycloalkyl, aryl, heterocyclyl and heteroaryl; or
R⁷ and R⁸ taken together with the carbon to which they are attached to form a cycloalkyl attached to the piperazine; or
R⁹ and R¹⁰ taken together with the carbon atom to which they are attached form a cycloalkyl attached to the piperazine; or
R⁷ and R⁹ taken together with the carbon to which they are attached to form a cycloalkyl group attached to the piperazine; or
R⁸ and R¹⁰ taken together with the carbon atoms to which they are attached form a cycloalkyl group attached to the piperazine;
   provided that R³ and R⁴ are not cycloalkyl, aryl, heteroaryl or heterocyclyl at the same time;
   provided that R⁵ and R⁶ are not cycloalkyl, aryl, heteroaryl or heterocyclyl at the same time;
   provided that R⁷, R⁸, R⁹ and R¹⁰ are not cycloalkyl, aryl, heteroaryl or heterocyclyl at the same time;
R^{a} and R^{b} are independently selected from H, alkyl, cycloalkyl and aryl;
R^{c} is selected from the optionally substituted group consisting of alkyl, alkoxy, alkoxyalkyl, amino, alkyl-C(O)—NR^{a}R^{b}, cycloalkyl, cycloalkylalkyl, NR^{a}R^{b}, alkyl-NR^{a}R^{b}, —NH-alkyl-NR^{a}R^{b}, heteroaryl, heteroarylalkyl, heterocyclyl, heterocyclylalkyl, phenylalkylamino, —NH-heteroaryl and —NH-heterocyclyl
R^{d} is selected from the optionally substituted group consisting of —(CH₂)ₘ—NH₂, alkyl, alkoxy, aryl, heteroaryl, heterocyclyl and arylalkyl;
m is 1 or 2; and
n is 1, 2, 3 or 4; or
Q is O; and
R¹ is selected from the optionally substituted group consisting of alkyl, —(CH₂)ₙC(O)—OCH₃, —(CH₂)ₙC(O)—O-alkyl, C(O)-phenyl, adamantanyl, benzo[1,3]dioxolyl, benzyl, cyclohexyl, cyclopentyl, diphenylmethyl, fluorenyl, indanyl, isoquinolinyl, diphenylmethyl, naphthyl, phenyl, piperidinyl, quinolinyl and thienyl; or
R¹ is A-B wherein
   A is selected from the optionally substituted group consisting of methyl, ethyl, phenoxy and phenyl;
   B is selected from the optionally substituted group consisting of benzyloxy, furanyl, phenoxy and phenyl;
X is selected from the group consisting of a bond, C(O) and C(O)NH;
R² is selected from the optionally substituted group consisting of benzimidazolyl, benzoxazolyl, benzyl. cyclohexyl, phenyl, piperidinyl, pyrazinyl, pyridazinyl, pyrimidinyl, pyrrolo[3,2-d]pyrimidinyl, quinazolinyl quinolinyl, quinoxalinyl and thieno[3,2-d]pyrimidinyl; or
R² is Y-Z wherein Y is attached to X and
   Y is selected from the group consisting of naphthyl, phenyl, pyridazinyl, pyrimidinyl, tetrazolyl and 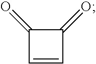
   Z is selected from the optionally substituted group consisting of phenoxy, phenyl and piperazinyl;
R³ is H or isopropyl;
R⁴, R⁵, R⁷, R⁸ and R¹⁰ are H;
R⁶ is H, isopropyl or phenyl;
R⁹ is H, isopropyl or optionally substituted phenyl;
provided that X—R² is not H;
provided that the compound is not 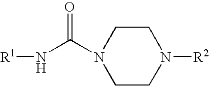
wherein
R¹ is phenyl substituted with CF₃, piperidinyl substituted with methyl, cyclohexyl substituted with one or more C(O)OH, methyl, CF₃, methoxy, F, Cl or H or 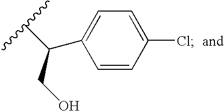
R² is benzyl, phenyl optionally substituted with one or more OH, Cl or methoxy, piperidinyl substituted with methyl, pyrimidinyl substituted with Cl or quinolinyl substituted with Cl;
provided that the compound is not 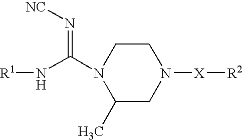
wherein
X is a bond;
R¹ is selected from the group consisting of methyl, ethyl, t-butyl, butyl, cyclohexyl, furanylmethyl, pyridinylmethyl, pyridinylethyl, optionally substituted phenyl, optionally substituted benzyl, optionally substituted phenylethyl, optionally substituted phenylpropyl, 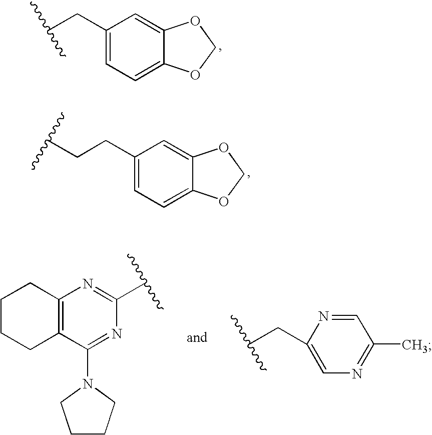
wherein the substituents are selected from the group consisting of Cl, Br, F, methyl, propyl, isobutyl, butyl, t-butyl, OCH3, isopropoxy, O-t-butyl, cyclohexyl, CF₃, SCH₃, SO₂CH₃ and CH₂C(CF₃)₃; and
R² is selected from the group consisting of phenylethyl, 1,2,3-triazolyl, pyridinyl substituted with Cl, quinolinyl substituted with Cl, 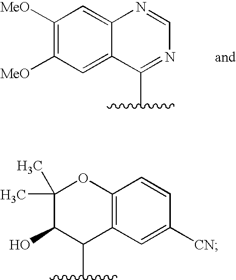
provided that the compound is not 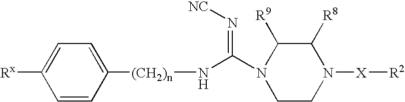
wherein
X is a bond;
R^{x} is selected the group consisting of from t-butyl, isobutyl, sec-butyl, t-butoxy, isopropyl, CF₃, ethyl, OCF₃, halo, n-butyl and n-propyl;
R⁸ and R⁹ are independently H or methyl;
R² is selected from the group consisting of 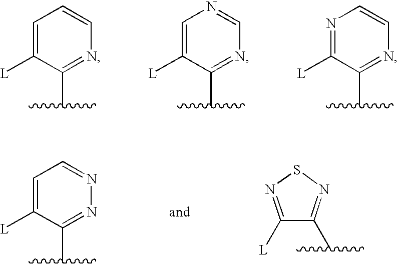
wherein L is Cl, methyl, CF₃, OH, NO₂, CN, Br, I or F; and
n is 0, 2 or 3.

In a second embodiment the invention provides pharmaceutically acceptable salts thereof, metabolites thereof, isomers thereof, enantiomers thereof or prodrugs thereof wherein Q is O.

In a third embodiment the invention provides pharmaceutically acceptable salts thereof, metabolites thereof, isomers thereof, enantiomers thereof or prodrugs thereof according to any of the foregoing inventions wherein X is C(O).

In a fourth embodiment the invention provides pharmaceutically acceptable salts thereof, metabolites thereof, isomers thereof, enantiomers thereof or prodrugs thereof according to any of the foregoing inventions wherein
R¹ is selected from optionally substituted group consisting of adamantanyl, benzo[1,3]dioxolyl, benzyl, butyl, t-butyl, C(O)-phenyl, cyclohexyl, cyclopentyl, diphenylmethyl, ethyl, fluorenyl, naphthyl and phenyl; or
R¹ is A-B wherein
   A is selected from the group consisting of ethyl and phenyl; and
   B is selected from the group consisting of benzyloxy, phenoxy and phenyl; and
   R² is selected from the optionally substituted group consisting of phenyl and benzyl.

In a fifth embodiment the invention provides pharmaceutically acceptable salts thereof, metabolites thereof, isomers thereof, enantiomers thereof or prodrugs thereof according to any of the foregoing inventions wherein
R¹ is selected from the optionally substituted group consisting of t-butyl, adamantanyl, benzo[1,3]dioxolyl, benzyl, C(O)-phenyl, cyclohexyl, cyclopentyl, diphenylmethyl, fluorenyl, naphthyl and phenyl; or
R¹ is A-B wherein
   A is selected from the group consisting of ethyl and phenyl; and
   B is selected from the group consisting of benzyloxy, phenoxy and phenyl;
R⁶ is phenyl; and
R³ and R⁹ are H or isopropyl.

In a sixth embodiment the invention provides pharmaceutically acceptable salts thereof, metabolites thereof, isomers thereof, enantiomers thereof or prodrugs thereof according to any of the foregoing inventions wherein
R¹ is selected from the optionally substituted group consisting of benzyl, naphthyl and phenyl;
   wherein the benzyl is substituted with methyl or Cl and the phenyl is optionally substituted with one or more methyls;
R² is benzyl substituted with two OCH₃; and
R⁶ is H.

In a seventh embodiment the invention provides pharmaceutically acceptable salts thereof, metabolites thereof, isomers thereof, enantiomers thereof or prodrugs thereof according to first or second embodiment of the invention wherein X is a bond.

In an eighth embodiment the invention provides pharmaceutically acceptable salts thereof, metabolites thereof, isomers thereof, enantiomers thereof or prodrugs thereof according to the seventh embodiment of the invention wherein
R¹ is selected from the optionally substituted group consisting of methyl, ethyl, propyl, butyl, t-butyl, pentyl, CH₂CH₂C(O)—OCH₂CH₃, adamantanyl, benzyl, cyclohexyl, indanyl, naphthyl, phenyl, piperidinyl, quinolinyl and thienyl; or
R¹ is A-B wherein
   A is selected from the optionally substituted group consisting of methyl, ethyl phenoxy and phenyl; and
   B is selected from the optionally substituted group consisting of furanyl, phenoxy and phenyl;
   R² is selected from the optionally substituted group consisting of benzimidazolyl, benzoxazolyl, benzyl, cyclohexyl, pyrazinyl, pyridazinyl, pyrimidinyl, pyrrolo[3,2-d]pyrimidinyl, quinazolinyl and thieno[3,2-d]pyridinyl; or
R² is Y-Z wherein
   Y is selected from the optionally substituted group consisting of naphthyl, phenyl, pyridazinyl, pyrimidinyl, tetrazolyl and 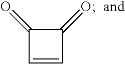
   Z is selected from the optionally substituted group consisting of phenyl and piperazinyl.
R³ is isopropyl; and
R⁹ is H, isopropyl or phenyl.

In a ninth embodiment the invention provides pharmaceutically acceptable salts thereof, metabolites thereof, isomers thereof, enantiomers thereof or prodrugs thereof according to the seventh and eighth embodiments of the invention wherein
R¹ is selected from the optionally substituted group consisting of methyl, propyl, butyl, CH₂CH₂C(O)—OCH₂CH₃, adamantanyl, benzyl, cyclohexyl, indanyl, naphthyl, phenyl, piperidinyl, quinolinyl and thienyl; or
R¹ is A-B wherein
   A is selected from the optionally substituted group consisting of methyl, ethyl and phenyl; and
   B is selected from the optionally substituted group consisting of furanyl, phenoxy and phenyl;
R² is selected from the optionally substituted group consisting of benzimidazolyl, benzoxazolyl, pyrazinyl, pyridazinyl, pyrimidinyl, pyrrolo[3,2-d]pyrimidinyl, quinazolinyl and thieno[3,2-d]pyrimidinyl; or
R² is Y-Z wherein Y is selected from the optionally substituted group consisting of pyridazinyl, pyrimidinyl and tetrazolyl; and
Z is selected from the optionally substituted group consisting of phenyl and piperazinyl.

In a tenth embodiment the invention provides pharmaceutically acceptable salts thereof, metabolites thereof, isomers thereof, enantiomers thereof or prodrugs thereof according to the seventh through ninth embodiments of the invention wherein
R¹ is selected from the optionally substituted group consisting of naphthyl, phenyl and quinolinyl;
R² is selected from the optionally substituted group consisting of pyrazinyl, pyridazinyl, pyrimidinyl, pyrrolo[3,2-d]pyrimidinyl and thieno[3,2-d]pyrimidinyl; or
R² is Y-Z wherein Y is pyridazinyl and Z is phenyl.

In an eleventh embodiment the invention provides pharmaceutically acceptable salts thereof, metabolites thereof, isomers thereof, enantiomers thereof or prodrugs thereof according to the second embodiment of the invention wherein X is C(O)NH wherein the C(O) is attached to the nitrogen of the piperazine.

In a twelfth embodiment the invention provides pharmaceutically acceptable salts thereof, metabolites thereof, isomers thereof, enantiomers thereof or prodrugs thereof according to the second, tenth and eleventh embodiments of the invention wherein R¹ is selected from the optionally substituted group consisting of naphthyl and benzyl;
R² is 4-chlorophenyl;
R³, R⁴, R⁵, R⁶, R¹, R⁸ and R¹⁰ are H; and
R⁹ is isopropyl.

In a thirteenth embodiment the invention provides pharmaceutically acceptable salts thereof, metabolites thereof, isomers thereof, enantiomers thereof or prodrugs thereof according to the second and tenth through twelfth embodiments of the invention wherein R¹ is benzyl substituted with methyl or Cl.

In a fourteenth embodiment the invention provides pharmaceutically acceptable salts thereof, metabolites thereof, isomers thereof, enantiomers thereof or prodrugs thereof according to the first embodiment of the invention wherein Q is S.

In a fifteenth embodiment the invention provides pharmaceutically acceptable salts thereof, metabolites thereof, isomers thereof, enantiomers thereof or prodrugs thereof according to the first and fourteenth embodiments of the invention wherein X is a bond.

In a sixteenth embodiment the invention provides pharmaceutically acceptable salts thereof, metabolites thereof, isomers thereof, enantiomers thereof or prodrugs thereof according to the first and fifteenth embodiments of the invention wherein R¹ is selected from the optionally substituted group consisting of indazolyl, indolyl, phenyl and quinolinyl; or
R¹ is A-B wherein A is phenyl and B is C(O)—NR^{a}R^{b};
R² is selected from the optionally substituted group consisting of benzoxazolyl, benzimidazolyl, pyrazinyl, pyridazinyl, pyridinyl, pyrimidinyl, pyrrolo[2,3-d]pyrimidinyl; or
R² is Y-Z wherein
   Y is selected from the optionally substituted group consisting of pyrazinyl, pyridazinyl, pyrimidinyl and quinazolinyl; and
   Z is NH(CH₂)ₙNR^{a}R^{b}, NH(CH₂)ₙO-alkyl, NH(CH₂)ₙOH or NH(CH₂)ₙS(O) alkyl; or
   Z is selected from the optionally substituted group consisting of NH(CH₂)ₙ-morpholino, NH(CH₂)ₙS(O)₂(CH₂)ₙ-pyrrolidinyl, 4-methoxybenzylamino, C(O)-morpholinyl, C(O)—OCH₂-phenyl, 2,3-dihydrobenzofuranyl, benzo[1,2,5]oxadiazolyl, benzo[b]thiophenyl, benzylamino, indolyl, isoxazolyl, morpholinyl, phenyl, piperazinyl, piperidinyl, pyrazolyl, pyridinyl, pyrimidinyl, pyrrolyl, quinolinyl, thieno[3,2-d]pyrimidinyl and thienyl;
R³ and R⁹ are independently selected from H and isopropyl;
R⁵ and R⁶ are independently selected from H and methyl;
R⁴, R⁷, R⁸ and R¹⁰ are H;
R^{a} and R^{b} are independently selected from H and methyl.

In a seventeenth embodiment the invention provides pharmaceutically acceptable salts thereof, metabolites thereof, isomers thereof, enantiomers thereof or prodrugs thereof according to the first and fourteenth through sixteenth embodiments of the invention wherein
R² is selected from the optionally substituted group consisting of benzoxazolyl, benzimidazolyl, pyrazinyl, pyridazinyl, pyridinyl, pyrimidinyl and pyrrolo[2,3-d]pyrimidinyl; or
R² is Y-Z wherein
   Y is selected from the optionally substituted group consisting of pyrazinyl, pyridazinyl and pyrimidinyl; and
   Z is —NH(CH₂)ₙN(CH₃)₂, —NH(CH₂)ₙOCH₃, —NH(CH₂)ₙOH or —NH(CH₂)ₙ—S(O)alkyl; or
   Z is selected from the optionally substituted group consisting of —NH(CH₂)ₙ-morpholino, 4-methoxybenzylamino, C(O)-morpholinyl, —C(O)—OCH₂-phenyl, —NH(CH₂)ₙS(O)₂CH₂-pyrrolidinyl, 2,3-dihydrobenzofuranyl, benzo[1,2,5]oxadiazolyl, benzo[b]thiophenyl, benzylamino, indolyl, isoxazolyl, morpholinyl, phenyl, piperazinyl, pyrazolyl, pyridinyl, pyrimidinyl, pyrrolyl, quinolinyl, thieno[3,2-d]pyrimidinyl and thienyl;
R^{a} and R^{b} are each methyl; and
n is 1, 2 or 3.

In an eighteenth embodiment the invention provides pharmaceutically acceptable salts thereof, metabolites thereof, isomers thereof, enantiomers thereof or prodrugs thereof according to the first and fourteenth through seventeenth embodiments of the invention wherein
R² is selected from the optionally substituted group consisting of benzimidazolyl, pyrazinyl, pyridazinyl, pyridinyl, pyrimidinyl and pyrrolo[2,3-d]pyrimidinyl; or
R² is Y-Z wherein
Y is selected from the optionally substituted group consisting of pyrazinyl, pyridazinyl and pyrimidinyl; and
Z is —NHCH₂CH₂S(O)₂CH₂CH₃, C(O)NHCH₂CH₂OH; or
Z is selected from the optionally substituted group consisting of C(O)-morpholinyl, —C(O)—OCH₂-phenyl, —NHCH₂CH₂-morpholino, benzo[b]thiophenyl, isoxazolyl, morpholinyl, piperazinyl, pyrazolyl, pyridinyl, 4-methoxybenzylamino, phenyl, piperazinyl, pyridinyl, pyrimidinyl, pyrrolyl, quinolinyl, thieno[3,2-d]pyrimidinyl and thienyl.

In a nineteenth embodiment, the invention provides pharmaceutically acceptable salts thereof, metabolites thereof, isomers thereof, enantiomers thereof or prodrugs thereof according to the first and fourteenth through eighteenth embodiments of the invention wherein
R¹ is quinolinyl;
R² is Y-Z wherein
   Y is pyridazinyl; and
   Z is benzo[b]thiophenyl substituted with methyl;
R³ is isopropyl; and
R⁴, R⁵, R⁶, R⁷, R⁸, R⁹, R¹⁰ are H.

In a twentieth embodiment, the invention provides pharmaceutically acceptable salts thereof, metabolites thereof, isomers thereof, enantiomers thereof or prodrugs thereof according to the first, and fourteenth through nineteenth embodiments of the invention wherein X is C(O)—NH wherein the C(O) is attached to the nitrogen of the piperazine.

In a twenty-first embodiment the invention provides pharmaceutically acceptable salts thereof, metabolites thereof, isomers thereof, enantiomers thereof or prodrugs thereof. according to the first and fourteenth through twentieth embodiments of the invention wherein R¹ is 2,3-dihydrobenzofuranyl, quinolinyl or phenyl wherein
the quinolinyl is optionally substituted with methyl and the phenyl is substituted with —C(O)OCH₃ or —S(O)₂CH₃;
R² is phenyl substituted with Cl, F or CF₃;
R³ is H or isopropyl;
R⁴, R⁵, R⁶, R⁷, R⁸ and R¹⁰ are H; and
R⁹ is H or isopropyl.

In a twenty-second embodiment the invention provides pharmaceutically acceptable salts thereof, metabolites thereof, isomers thereof, enantiomers thereof or prodrugs thereof according to the first embodiment wherein Q is N—CN.

In a twenty-third embodiment the invention provides pharmaceutically acceptable salts thereof, metabolites thereof, isomers thereof, enantiomers thereof or prodrugs thereof according to the first and twenty-second embodiments wherein X is a bond.

In a twenty-fourth embodiment the invention provides pharmaceutically acceptable salts thereof, metabolites thereof, isomers thereof, enantiomers thereof or prodrugs thereof according to the first, twenty-second and twenty-third embodiments wherein
R¹ is selected from the optionally substituted group consisting of dihydrobenzofuranyl, indazolyl, isoquinolinyl, isoxazolo[5,4-b]pyridinyl, phenyl, quinolinyl and tetrahydroquinolinyl; or
R¹ is A-B wherein
   A is selected from the optionally substituted group consisting of isoquinolinyl, phenyl and pyrazinyl;
   B is —C(O)—OCH₂-phenyl, —C(O)—OCH₂-fluorene, —C(O)—R^{c} or —C(O)—NR^{a}R^{b};
R² is selected from the optionally substituted group consisting of benzimidazolyl, benzo[4,5]thieno[3,2-d]pyrimidinyl, benzoxazolyl, benzothiazolyl, imidazo[1,2-b]pyridazinyl, pyrazinyl, pyridazinyl, pyridinyl, pyrimidinyl, pyrrolo[3,2-d]pyrimidinyl, quinazolinyl, quinoxalinyl, thiazolyl, thieno[2,3-d]pyrimidinyl, thieno[3,2-d]pyrimidinyl and thienyl; or
R² is Y-Z wherein
   Y is selected from the optionally substituted group consisting of pyrazinyl, pyridazinyl, pyrimidinyl, quinazolinyl, tetrazolyl, tetrahydroquinolinyl, thieno[3,2-d]pyrimidinyl (opt subs w/t-butyl) and 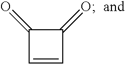
   Z is selected from the optionally substituted group consisting of —C(O)—R^{d}, —C(O)—NR^{a}R^{b}, —C(O)NH(CH₂)ₙOH, —C(O)—O(CH₂)ₙ-phenyl, —NH(CH₂)ₙ-morpholino, —NH(CH₂)ₙN(CH₃)₂, —NH(CH₂)ₙN(alkyl)₂, —NH(CH₂)ₙOCH₃, —NH(CH₂)ₙ, morpholinyl, phenyl, piperazinyl, pyrazolyl and pyridinyl;
R³ and R⁷ are H or isopropyl;
R⁸ is H or phenyl;
R⁹ is isopropyl or phenyl;
R⁴, R⁵, R⁶ and R¹⁰ are H;
R^{a} and R^{b} are independently selected from H and methyl;
R^{c} is selected from the optionally substituted group consisting of alkyl and amino; and
R^{d} is selected from the optionally substituted group consisting of alkyl, alkoxy, aryl, heteroaryl and heterocyclyl.

In a twenty-fifth embodiment the invention provides pharmaceutically acceptable salts thereof, metabolites thereof, isomers thereof, enantiomers thereof or prodrugs thereof according to the first and twenty-second through twenty-fourth embodiments of the invention wherein
R¹ is selected from the optionally substituted group consisting of dihydrobenzofuranyl, indazolyl, isoquinolinyl, isoxazolo[5,4-b]pyridinyl, phenyl, quinolinyl and tetrahydroquinolinyl; or
R¹ is A-B wherein
   A is isoquinolinyl, pyrazinyl or optionally substituted phenyl; and
   B is C(O)—OCH₂-phenyl, C(O)-alkyl or C(O)—NR^{a}R^{b};
R² is selected from the optionally substituted group consisting of benzimidazolyl, benzo[4,5]thieno[3,2-d]pyrimidinyl, benzoxazolyl, benzothiazolyl, imidazo[1,2-b]pyridazinyl, pyrazinyl, pyridazinyl, pyridinyl, pyrimidinyl, pyrrolo[3,2-d]pyrimidinyl, quinazolinyl, quinoxalinyl, thiazolyl, thieno[2,3-d]pyrimidinyl, thieno[3,2-d]pyrimidinyl and thienyl; or
R² is Y-Z wherein
   Y is selected from the optionally substituted group consisting of pyrazinyl, pyridazinyl, pyrimidinyl, quinazolinyl, tetrazolyl and thieno[3,2-d]pyrimidinyl and 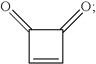
   Z is selected from the optionally substituted group consisting of —C(O)—R^{d}, —C(O)—NR^{a}R^{b}, —C(O)NHCH₂CH₂N(CH₃)₂, —C(O)NHCH₂CH₂OH, —C(O)—O—CH₂-phenyl, —NHCH₂CH₂-morpholino, —NHCH₂CH₂N(CH₃)₂, —NHCH₂CH₂N(CH₃)₂, —NHCH₂CH₂CH₂N(CH₃)₂, —NHCH₂CH₂OCH₃, —NHCH₂CH₂OH, morpholinyl, phenyl, piperazinyl, pyrazolyl, pyridinyl; and
R^{d} is selected from the optionally substituted group consisting of alkoxy, alkyl, isoxazolyl, morpholinyl, phenyl, piperazinyl, piperidinyl, pyridinyl, pyrrolidinyl, thiazolyl and triazolyl.

In a twenty-sixth embodiment the invention provides pharmaceutically acceptable salts thereof, metabolites thereof, isomers thereof, enantiomers thereof or prodrugs thereof according to the first and twenty-second through twenty-fifth embodiments of the invention wherein
R¹ is selected from the optionally substituted group consisting of dihydrobenzofuranyl, indazolyl, isoquinolinyl, phenyl, quinolinyl and tetrahydroquinolinyl; or
R¹ is A-B wherein
   A is unsubstituted isoquinolinyl or phenyl substituted with methyl; and
   B is selected from the group consisting of —C(O)—OCH₂-phenyl, —C(O)—CH₃ and —C(O)—N(CH₃)₂;
R² is selected from the optionally substituted group consisting of benzimidazolyl, benzoxazolyl, imidazo[1,2-b]pyridazinyl, pyrazinyl, pyridazinyl, pyridinyl, pyrimidinyl, pyrrolo[3,2-d]pyrimidinyl, quinazolinyl, quinoxalinyl, thiazolyl, thieno[2,3-d]pyrimidinyl, thieno[3,2-d]pyrimidinyl and thienyl; or
R² is Y-Z wherein
   Y is selected from the optionally substituted group consisting of pyrazinyl, pyridazinyl, pyrimidinyl and 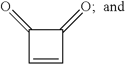
   Z is selected from the optionally substituted group consisting of C(O)-morpholinyl, C(O)-piperazinyl, C(O)-piperidinyl, C(O)-pyrrolidinyl, —C(O)—NH-isoxazolyl, —C(O)—NH-phenyl, —C(O)—NH-pyridinyl, —C(O)—NH-thiazolyl, —C(O)NHCH₂CH₂OH, NHCH₂CH₂-morpholino, phenyl, piperazinyl, pyrazolyl and pyridinyl; and
R⁹ is isopropyl.

In a twenty-seventh embodiment pharmaceutically acceptable salts thereof, metabolites thereof, isomers thereof, enantiomers thereof or prodrugs thereof according to the first and twenty-second embodiments of the invention wherein X is C(O)NH wherein the C(O) is attached to the piperazine.

In a twenty-eighth embodiment pharmaceutically acceptable salts thereof, metabolites thereof, isomers thereof, enantiomers thereof or prodrugs thereof according to the first, twenty-second and twenty-seventh embodiments of the invention wherein
R¹ is selected from the optionally substituted group consisting of C(O)-cycloalkyl, benzimidazolyl, benzo[1,3]dioxazolyl, benzothiazolyl, benzyl, cinnolinyl, cyclopropyl, dihydrobenzofuranyl, alkyl indazolyl, indolyl, naphthyl, phenyl, pyrazolyl, pyridinyl, quinolinyl, tetrahydronaphthyl and tetrahydroquinolinyl; or
R¹ is A-B wherein
   A is selected from the optionally substituted group consisting of alkylidenyl, isoquinolinyl, phenyl, pyridinyl and tetrahydroquinolinyl;
   B is selected from the optionally substituted group consisting of —C(O)—R^{c}, —C(O)CH₂CH₂—C(O)—NH₂, —NH—C(O)-cycloalkyl, —NH—C(O)—(CH₂)₂-cycloalkyl, cycloalkylalkyl, cycloalkyl, heteroaryl, heterocyclyl, heterocycloalkyl, phenyl and —NH—C(O)—OCH₂-phenyl;
R² is selected from the optionally substituted group consisting of benzo[1,3]dioxazolyl, benzo[1,2,5]thiadiazolyl, benzothiazolyl, benzo[b]thienyl 1,1,dioxide, benzyl, —CH₂C(O)OCH₂CH₃, cyclohexyl, dihydrobenzo[1,4]dioxinyl, alkyl, indanyl, isoxazolyl, naphthyl, phenyl and thienyl; or
R² is Y-Z wherein
   Y is selected from the optionally substituted group consisting of alkylidenyl, phenyl and thienyl;
   Z is selected from the optionally substituted group consisting of benzyl, benzyloxy, C(O)-heterocyclyl, phenoxy, phenyl, pyridinyl, thienyl, —CH₂—NH—C(O)—OCH₂-phenyl
R³ is H or is selected from the optionally substituted group consisting of alkyl, cycloalkyl, and phenyl;
R⁴ and R⁶ are H or methyl;
R⁷ is H or is selected from the optionally substituted group consisting of alkyl, furanyl, pyridinyl, phenyl and thienyl;
R⁸ is H or alkyl;
R⁹ is H, alkyl or phenyl; or
R⁷ and R⁸ taken together taken together with the carbon atom to which they are attached form a cyclopropyl, cyclobutyl, cyclopentyl or cyclohexyl attached to the piperazine;
R^{c} is selected from the optionally substituted group consisting of cycloalkyl, cycloalkylalkyl, heteroarylalkyl, heterocyclyl and heterocyclylalkyl.

In a twenty-ninth embodiment, the invention provides pharmaceutically acceptable salts thereof, metabolites thereof, isomers thereof, enantiomers thereof or prodrugs thereof according to the first, twenty-second, twenty-seventh and twenty-eight embodiments wherein
R¹ is selected from the optionally substituted group consisting of benzimidazolyl, benzo[1,3]dioxazolyl, benzyl, cinnolinyl, cyclopropyl, dihydrobenzofuranyl, ethyl, indazolyl, indolyl, naphthyl, phenyl, pyrazolyl, pyridinyl, quinolinyl, tetrahydronaphthyl and tetrahydroquinolinyl; or
R¹ is A-B wherein
   A is attached to the piperazine and A is selected from the optionally substituted group consisting of alkylidenyl, isoquinolinyl, phenyl, pyridinyl and tetrahydroquinolinyl;
   B is selected from the optionally substituted group consisting of —C(O)CH₂CH₂-cyclopentyl, —C(O)CH₂-cyclopropyl, —C(O)CH₂CH₂-imidazolyl, —C(O)CH₂CH₂-piperidinyl, —C(O)CH₂CH₂—C(O)—NH₂, C(O)-tetrahydropyranyl, C(O)-cyclopropyl, —C(O)—CH₂-pyridinyl, —NH—C(O)-cyclopropyl, —NH—C(O)—CH₂CH₂-cyclopentyl, cyclohexylmethyl, cyclopropyl, cyclopropylmethyl, morpholinyl, morpholinylmethyl, morpholinylethyl, oxazolyl, pentylmethyl, phenyl, piperidinylmethyl, pyridinyl, pyrrolidinylmethyl, tetrahydropyranyl or —NH—C(O)—OCH₂-phenyl;
R² is selected from the optionally substituted group consisting of benzo[1,3]dioxazolyl, benzo[1,2,5]thiadiazolyl, benzo[b]thienyl 1,1,dioxide, benzothiazolyl, benzyl, butyl, t-butyl, cyclohexyl, —CH₂C(O)OCH₂CH₃, dihydrobenzo[1,4]dioxinyl, ethyl, indanyl, isopropyl, isoxazolyl, naphthyl, phenyl and thienyl; or
R² is Y-Z wherein
   Y is selected from the optionally substituted group consisting of methyl, ethyl, phenyl and thienyl; and
   Z is selected from the optionally substituted group consisting of benzyl, benzyloxy, C(O)-morpholinyl, C(O)-piperazinyl, phenoxy, phenyl, thienyl and —CH₂—NHC(O)—OCH₂-phenyl
R³ is H or is selected from the optionally substituted group consisting of methyl, isopropyl, propyl, cyclopropyl, isobutyl and phenyl;
R⁴ is H or methyl;
R⁵ is selected from the group consisting of H, isopropyl, methyl and phenyl;
R⁶ is H, methyl or phenyl;
R⁷ is H or is selected from the optionally substituted group consisting of methyl, ethyl, isopropyl, propyl, butyl, isobutyl, propyl, pentyl, C(O)NH₂, furanyl, pyridinyl, phenyl and thienyl;
R⁸ is H, methyl, ethyl or propyl;
R⁹ is H, methyl, isopropyl or phenyl; or
R⁷ and R⁸ taken together taken together with the carbon atom to which they are attached form a cyclopropyl, cyclobutyl, cyclopentyl or cyclohexyl attached to the piperazine.

In a thirtieth embodiment the invention provides pharmaceutically acceptable salts thereof, metabolites thereof, isomers thereof, enantiomers thereof or prodrugs thereof according to first, twenty-second and twenty-eighth through twenty-ninth embodiments of the invention wherein
R¹ is selected from the optionally substituted group consisting of indolyl, phenyl, pyridinyl and quinolinyl or
R¹ is A-B wherein
   A is methyl or optionally substituted tetrahydroquinolinyl; and
   B is selected from the optionally substituted group consisting of —C(O)CH₂-cyclopropyl, cyclopropylmethyl and pyridinyl;
R² is selected from the optionally substituted group consisting of benzo[1,3]dioxazolyl, benzo[1,2,5]thiadiazolyl, benzo[b]thienyl 1,1,dioxide, benzyl, dihydrobenzo[1,4]dioxinyl, indanyl, isopropyl, isoxazolyl, naphthyl, phenyl and thienyl; or
R² is Y-Z wherein
   Y is selected from the optionally substituted group consisting of methyl, ethyl and phenyl; and
   Z is selected from the optionally substituted group consisting of C(O)-morpholinyl, phenyl, thienyl and —CH₂—NH—C(O)—OCH₂-phenyl;
   R³ is H or isopropyl;
   R⁵ is H, isopropyl or phenyl;
   R⁶ is H;
   R⁷ is H, methyl, isopropyl, isobutyl, propyl, C(O)NH₂ or phenyl;
R⁸ is H or methyl; and
R⁹ is isopropyl;

In a thirty-first embodiment, the invention provides pharmaceutically acceptable salts thereof, metabolites thereof, isomers thereof, enantiomers thereof or prodrugs thereof according to the first, twenty-second and twenty-eighth through thirtieth embodiments wherein
R¹ is phenyl substituted with methyl or R¹ is unsubstituted quinolinyl;
R² is unsubstituted dihydrobenzo[1,4]dioxinyl, unsubstituted thienyl or phenyl substituted with one or more CN, Cl, F, SO₂CH₃ or OCH₃; or
R² is Y-Z wherein Y is ethyl and Z is phenyl substituted with one or more C(O)CH₃ or OCH₃, or Z is unsubstituted CH₂—NH—C(O)—OCH₂-phenyl;
R³ is isopropyl; and
R⁷ is phenyl.

In a thirty-second embodiment, the invention provides pharmaceutically acceptable salts thereof, metabolites thereof, isomers thereof, enantiomers thereof or prodrugs thereof according to the first, twenty-second and twenty-eighth through thirtieth-first embodiments wherein X is C(O).

In a thirty-third embodiment, the invention provides pharmaceutically acceptable salts thereof, metabolites thereof, isomers thereof, enantiomers thereof or prodrugs thereof according to the first, twenty-second and twenty-eighth through thirty-second embodiments wherein
R¹ is selected from the optionally substituted group consisting of phenyl and quinolinyl;
R² is selected from the optionally substituted group consisting of alkyl, aryl, heteroaryl, heterocyclyl and —NH—CH₂—C(O)—O-alkyl; or
R² is Y-Z wherein
   Y is selected from the optionally substituted group consisting of alkylidenyl, heteroaryl and heterocyclyl; and
   Z is selected from the optionally substituted group consisting of [1,2,4]triazolyl, benzotriazolyl, furanyl, imidazolyl, indolyl, isoxazolyl naphthyl, morpholinyl, oxazolyl, phenoxy, phenyl, pyrazolyl, pyridinyl, quinolinyl, thiazolyl thienyl and —NH—CH₂—C(O)—O-alkyl;
R³ is H or methyl or isopropyl
R⁵ and R⁷ are independently selected from the group consisting of H, methyl, isopropyl and phenyl;
R⁸ is H or methyl; and
R⁹ is H, isopropyl or phenyl.

In a thirty-fourth embodiment, the invention provides pharmaceutically acceptable salts thereof, metabolites thereof, isomers thereof, enantiomers thereof or prodrugs thereof according to the first, twenty-second and twenty-eighth through thirty-third embodiments wherein
R¹ is selected from the optionally substituted group consisting of phenyl and quinolinyl;
R² is selected from the optionally substituted group consisting of [1,5]naphthyridinyl, [1,2,4]triazolo[1,5-a]pyrimidinyl, adamantanyl, benzofuranyl, benzo[b]thienyl, benzimidazolyl, benzo[1,2,5]oxadiazolyl, benzothiazolyl, benzotriazolyl, benzyl, chromenyl, cinnolinyl, furazanyl, furanyl, imidazolyl, imidazol[1,2-a]pyridinyl, indolyl, isoquinolinyl, isothiazolyl, isoxazolyl, methyl, phenyl, pyrazinyl, pyrazolo[1,5-a]pyrimidinyl, pyrazolyl, pyridinyl, pyrimidinyl, pyrrolidinyl, pyrrolyl, quinoxalinyl, tetrahydroindazolyl, thiazolyl, thienyl and NH—CH₂—C(O)—OCH₂CH₃; or
R² is Y—X wherein
   Y is selected from the optionally substituted group consisting of alkylidenyl, isoxazolyl, pyridinyl, pyrazolyl, thiazolyl and thienyl; and
   Z is selected from the optionally substituted group consisting of [1,2,4]triazolyl, benzotriazolyl, furanyl, imidazolyl, indolyl, isoxazolyl, morpholinyl, naphthyl, oxazolyl, phenoxy, phenyl, pyrazolyl, pyridinyl, thienyl and NH—CH₂—C(O)—OCH₂CH₃;
R⁵ is H, methyl or phenyl;
R⁷ is H, isopropyl or phenyl.

In a thirty-fifth embodiment the invention provides pharmaceutically acceptable salts thereof, metabolites thereof, isomers thereof, enantiomers thereof or prodrugs thereof according to the first, twenty-second, and thirty-second through thirty-fourth embodiments wherein
R² is selected from the optionally substituted group consisting of adamantanyl, benzyl, indolyl, phenyl, pyrazinyl, pyrazolyl, pyrrolyl, thiazolyl and thienyl; or
R² is Y-Z wherein
Y is selected from the optionally substituted group consisting of methyl, ethyl, propyl, pyridinyl and thienyl; and
Z is selected from the optionally substituted group consisting of benzotriazolyl, furanyl, isoxazolyl, morpholinyl, oxazolyl, phenyl, pyrazolyl, pyridinyl and thienyl;
R⁷ is isopropyl or phenyl; and
R⁹ is H, isopropyl or phenyl.

In a thirty-sixth embodiment the invention provides pharmaceutically acceptable salts thereof, metabolites thereof, isomers thereof, enantiomers thereof or prodrugs thereof according to the first and twenty-second embodiments wherein X is S(O)₂.

In a thirty-seventh embodiment the invention provides pharmaceutically acceptable salts thereof, metabolites thereof, isomers thereof, enantiomers thereof or prodrugs thereof according to the first, twenty-second and thirty-sixth embodiments wherein
R¹ is optionally substituted phenyl;
R² is selected from the optionally substituted group consisting of aryl, heteroaryl and heterocyclyl; or
R² is Y-Z wherein
   Y is selected from the optionally substituted group consisting of phenyl, pyrazolyl, pyridinyl and thienyl;
   Z is selected from the optionally substituted group consisting of isoxazolyl, morpholinyl, oxazolyl, phenoxy, phenyl, pyrazolyl, O-pyridinyl, quinolinyl and thiazolyl; and
R⁷ is isopropyl or phenyl.

In a thirty-eighth embodiment the invention provides pharmaceutically acceptable salts thereof, metabolites thereof, isomers thereof, enantiomers thereof or prodrugs thereof according to the first, twenty-second, thirty-sixth and thirty-seventh embodiments wherein
R¹ is phenyl substituted with one or more methyl, —NH—C(O)CH₃ or OCH₃,
R² is selected from the optionally substituted group consisting of benzo[1,2,5]oxadioazolyl, benzo[1,2,5]thiadiazolyl, benzo[b]thienyl, benzoxazolyl, benzyl, furanyl, imidazolyl, imidazol[2,1-b]thiazolyl, indolyl, isoquinolinyl, isoxazolyl, naphthyl, phenyl, pyrazolyl, thiazolyl and thienyl; or
R² is Y-Z wherein
   Y is selected from the optionally substituted group consisting of phenyl, pyrazolyl, pyridinyl and thienyl; and
   Z is selected from the optionally substituted group consisting of isoxazolyl, morpholinyl, oxazolyl, phenoxy, pyrazolyl, O-pyridinyl, quinolinyl and thiazolyl.

In a thirty-ninth embodiment the invention provides pharmaceutically acceptable salts thereof, metabolites thereof, isomers thereof, enantiomers thereof or prodrugs thereof according to the first, twenty-second and thirty-sixth through thirty-eighth embodiments wherein
R¹ is phenyl substituted with methyl or NH—C(O)CH₃;
R² is unsubstituted benzo[1,2,5]oxadioazolyl, unsubstituted benzo[1,2,5]thiadiazolyl, benzoxazolyl substituted with oxo, phenyl substituted with one or more methyl, F, CN, Cl, or OCH₃ or thienyl optionally substituted with methyl; and
R⁷ is phenyl.

### DETAILED DESCRIPTION OF THE INVENTION

A compound of formula (I) or a salt thereof or pharmaceutical compositions containing a therapeutically effective amount thereof is useful in the treatment of a disorder selected from the group comprising depression, rheumatoid arthritis, osteoarthritis, juvenile chronic arthritis, Lyme arthritis, psoriatic arthritis, reactive arthritis, and septic arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection (including but not limited to bone marrow and solid organ rejection), acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia areata, seronegative arthopathy, arthropathy, Reiter's disease, psoriatic arthropathy, ulcerative colitic arthropathy, enteropathic synovitis, chlamydia, yersinia and salmonella associated arthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis B, Hepatitis C, common varied immunodeficiency (common variable hypogammaglobulinaemia), dilated cardiomyopathy, female infertility, ovarian failure, premature ovarian failure, fibrotic lung disease, chronic wound healing, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, Lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjögren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo, acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci (GBS) infection, mental disorders (e.g., depression and schizophrenia), Th2 Type and Th1 Type mediated diseases, and cancers such as lung, breast, stomach, bladder, colon, pancreas, ovarian, prostate and rectal cancer and hematopoietic malignancies (leukemia and lymphoma), and hematopoietic malignancies (leukemia and lymphoma), and diseases involving inappropriate vascularization for example diabetic retinopathy, retinopathy of prematurity, choroidal neovascularization due to age-related macular degeneration, and infantile hemangiomas in human beings. In addition, such compounds may be useful in the treatment of disorders such as, edema, ascites, effusions, and exudates, including for example macular edema, cerebral edema, acute lung injury, adult respiratory distress syndrome (ARDS), proliferative disorders such as restenosis, fibrotic disorders such as hepatic cirrhosis and atherosclerosis, mesangial cell proliferative disorders such as glomerulonephritis, diabetic nephropathy, malignant nephrosclerosis, thrombotic microangiopathy syndromes, and glomerulopathies, myocardial angiogenesis, coronary and cerebral collaterals, ischemic limb angiogenesis, ischemia/reperfusion injury, peptic ulcer Helicobacter related diseases, virally-induced angiogenic disorders, Crow-Fukase syndrome (POEMS), preeclampsia, menometrorrhagia, cat scratch fever, rubeosis, neovascular glaucoma and retinopathies such as those associated with diabetic retinopathy, retinopathy of prematurity, or age-related macular degeneration, traumatic arthritis, rubella arthritis, acute synovitis, emphysema, bronchitis chronic obstructive pulmonary inflammatory disease, chronic pulmonary inflammatory disease, silicosis, pulmonary sarcoidosis, bronchitis chronic sarcoidosis, allergic reactions, allergic contact hypersensitivity, contact dermatitis, allergic contact dermatitis), sunburn, tissue ulceration, periodontal disease, epidermolysis bullosa, osteoporosis, bone resorption disease, loosening of artificial joint implants, aortic aneurysm, congestive heart failure, neurodegeneration, cerebral ischemia, head trauma, neurotrauma, spinal cord injury, migraine, peripheral neuropathy, pain, neuropathic pain, cerebral amyloid angiopathy, nootropic or cognition enhancement, amyotrophic lateral sclerosis, ocular angiogenesis, corneal injury, corneal scarring, scleritis, abnormal wound healing, chronic wound healing, burns, diabetes, endotoxic shock, conjunctivitis shock, conjunctivitis, gram negative sepsis, cerebral malaria, cardiac and renal reperfusion injury, thrombosis, organ transplant toxicity, organ transplant rejection, muscle degeneration, allergic dermatitis, hyperresponsiveness of the airway, irritable bowel disease, growth and metastases of malignant cells, myoblastic leukemia, bum injury, ischemic heart disease, varicose veins, an ocular condition, blastoma, teratocarcinoma, Abetalipoprotemia, Acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic rhinitis, alpha-1 antitrypsin deficiency, nemia, angina pectoris, anterior horn cell degeneration, anti cd3 therapy, antiphospholipid syndrome, anti-receptor hypersensitivity reactions, hypersensitivity reactions, hyperkinetic movement disorders, hypersensitivity pneumonitis, hypertension, hypokinetic movement disorders, aortic and peripheral aneurysms, hypothalamic-pituitary-adrenal axis evaluation, aortic dissection, arterial hypertension, arteriovenous fistula, ataxia, spinocerebellar degenerations, streptococcal myositis, structural lesions of the cerebellum, Subacute sclerosing panencephalitis, Syncope, syphilis of the cardiovascular system, systemic anaphylaxis, systemic inflammatory response syndrome, systemic onset juvenile rheumatoid arthritis, T-cell or FAB ALL, Telangiectasia, thromboangitis obliterans, transplants, trauma/hemorrhage, type III hypersensitivity reactions, type IV hypersensitivity, unstable angina, uremia, urosepsis, urticaria, valvular heart diseases, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue, atrial fibrillation (sustained or paroxysmal), atrial flutter, atrioventricular block, B cell lymphoma, bone graft rejection, bone marrow transplant (BMT) rejection, small bowel transplant rejection, spinal ataxia, bundle branch block, Burkitt's lymphoma, burns, cardiac arrhythmias, cardiac stun syndrome, cardiac tumors, cardiopulmonary bypass inflammation response, cartilage transplant rejection, cerebellar cortical degenerations, cerebellar disorders, chaotic or multifocal atrial tachycardia, chemotherapy associated disorders, chromic myelocytic leukemia (CML), chronic alcoholism, chronic inflammatory pathologies, chronic lymphocytic leukemia (CLL), chronic salicylate intoxication, colorectal carcinoma, conjunctivitis, cor pulmonale, coronary artery disease, Creutzfeldt-Jakob disease, culture negative sepsis, cystic fibrosis, cytokine therapy associated disorders, Dementia pugilistica, demyelinating diseases, dengue hemorrhagic fever, dermatologic conditions, diabetic ateriosclerotic disease, Diffuses Lewy body disease, dilated congestive cardiomyopathy, disorders of the basal ganglia, Down's Syndrome in middle age, drug-induced movement disorders induced by drugs which block CNS dopamine receptors, drug sensitivity, eczema, encephalomyelitis, endocarditis, endocrinopathy, epiglottitis, Epstein Barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, glomerular nephritis, gram positive sepsis, granulomas due to intracellular organisms, hairy cell leukemia, Hallerrorden-Spatz disease, hay fever, heart transplant rejection, hemachromatosis, hemodialysis, hemolytic uremic syndrome/thrombolytic thrombocytopenic purpura, hemorrhage, antibody mediated cytotoxicity, Asthenia, infantile spinal muscular atrophy, inflammation of the aorta, influenza A, ionizing radiation exposure, iridocyclitis/uveitis/optic neuritis, juvenile rheumatoid arthritis, juvenile spinal muscular atrophy, kidney transplant rejection, legionella, leishmaniasis, lipedema, liver transplant rejection, lymphederma, malaria, malignant Lymphoma, malignant histiocytosis, malignant melanoma, meningococcemia, metabolic/idiopathic, mitochondrial multi-system disorder, monoclonal gammopathy, multiple myeloma, multiple systems degenerations (Mencel Dejerine-Thomas Shi-Drager and Machado-Joseph), myasthenia gravis, mycobacterium avium intracellulare, mycobacterium tuberculosis, myelodyplastic syndrome, myocardial ischemic disorders, nasopharyngeal carcinoma, neonatal chronic lung disease, nephritis, nephrosis, neurogenic I muscular atrophies, neutropenic fever, non-hodgkins lymphoma, occlusion of the abdominal aorta and its branches, occulsive arterial disorders, okt3 therapy, orchitis/epidydimitis, orchitis/vasectomy reversal procedures, organomegaly, pancreas transplant rejection, pancreatic carcinoma, paraneoplastic syndrome/hypercalcemia of malignancy, parathyroid transplant rejection, pelvic inflammatory disease, perennial rhinitis, pericardial disease, Kaposi's sarcoma, Hodgkin's disease, myeloma, endometriosis, pulmonary hypertension, Herpes simplex, Herpes Zoster, human immunodeficiency virus, parapoxvirus, protozoa or toxoplasmosis, Progressive supranucleo Palsy, primary pulmonary hypertension, radiation therapy, Raynaud's phenomenon and disease, Refsum's disease, regular narrow QRS tachycardia, renovascular hypertension, restrictive cardiomyopathy, senile chorea, Senile Dementia of Lewy body type, shock, skin allograft, skin changes syndrome, ocular or macular edema, ocular neovascular disease, scieritis, radial keratotomy, Stargardt's disease, Eales disease, retinopathy, ischemic stroke, vascular occlusion, carotid obstructive disease, diabetes mellitus, peripheral vascular disorders, peritonitis, H is bundle arrythmias, HIV infection/HIV neuropathy, pneumocystis carinii pneumonia, pneumonia, acute and chronic pain (different forms of pain), toxicity, transplants, acute inflammatory demyelinating polyradiculoneuropathy, acute ischemia, adult Still's disease, anaphylaxis, anti-phospholipid antibody syndrome, aplastic anemia, atopic eczema, atopic dermatitis, autoimmune dermatitis, autoimmune diabetes, autoimmune disorder associated with streptococcus infection, autoimmune enteropathy, autoimmune hearing loss, autoimmune lymphoproliferative syndrome (ALPS), autoimmune myocarditis, autoimmune premature ovarian failure, autoimmune uveitis, Behcet's disease, blepharitis, bronchiectasis, bullous pemphigoid, catastrophic antiphospholipid syndrome, celiac disease, cervical spondylosis, chronic ischemia, cicatricial pemphigoid, clinical isolated syndrome (CIS) with risk for multiple sclerosis, childhood onset psychiatric disorder, dacrocystitis, dermatomyositis, disc herniation, disc prolapse, drug induced immune hemolytic anemia, endophthalmitis, episcleritis, erythema multiforme, erythema multiforme major, gestational pemphigoid, Guillain-Barre syndrome, heart failure, Hughes syndrome, idiopathic Parkinson's disease, idiopathic interstitial pneumonia, IgE-mediated allergy, immune hemolytic anemia, inclusion body myositis, infectious ocular inflammatory disease, inflammatory demyelinating disease, inflammatory heart disease, inflammatory kidney disease, IPF/UIP, iritis, keratitis, keratojuntivitis sicca, Kussmaul disease or Kussmaul-Meier disease, Landry's paralysis, Langerhan's cell hisiocytosis, livedo reticularis, microscopic polyangiitis, morbus bechterev, motor neuron disorders, mucous membrane pemphigoid, primary progressive multiple sclerosis, secondary progressive multiple sclerosis, relapsing remitting multiple sclerosis, multiple organ failure, myelodysplastic syndrome, nerve root disorder, neuropathy, Non-A Non-B hepatitis, osteolysis, ovarian cancer, pauciarticular JRA, peripheral artery occlusive disease (PAOD), periphral vascular disease (PVD), peripheral artery disease (PAD), phlebitis, polychondritis, polymyalgia rheumatica, poliosis, polyarticular JRA, polyendocrine deficiency syndrome, polymyositis, post-pump syndrome, primary parkinsonism, prostatitis, psoratic arthropathy, pure red cell aplasia, primary adrenal insufficiency, Reiter's disease, recurrent neuromyelitis optica, rheumatic heart disease, SAPHO (synovitis, acne, pustulosis, hyperostosis, and osteitis), scleroderma, secondary amyloidosis, shock lung, sciatica, secondary adrenal insufficency, septic arthritis, seronegative arthropathy, silicone associated connective tissue disease, Sneddon-Wilkinson Dermatosis, spondilitis ankylosans, Stevens-Johnson Syndrome (SJS), systemic inflammatory response syndrome, temporal arteritis, toxoplasmic retinitis, toxic epidermal necrolysis, TRAPS (Tumor Necrosis factor receptor), type 1 allergic reaction, type II diabetes, urticaria, usual interstitial pneumonia (UIP), vernal conjunctivitis, viral retinitis, Vogt-Koyanagi-Harada syndrome (VKH syndrome) and wet macular degeneration. In addition, these compounds can be used as active agents against solid tumors, malignant ascites, von Hippel Lindau disease, hematopoietic cancers and hyperproliferative disorders such as thyroid hyperplasia (especially Grave's disease), and cysts (such as hypervascularity of ovarian stroma characteristic of polycystic ovarian syndrome (Stein-Leventhal syndrome), and polycystic kidney disease since such diseases require a proliferation of blood vessel cells for growth and/or metastasis.

Compounds of formula (I) of the invention can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the compound of the present invention. The additional agent also can be an agent that imparts a beneficial attribute to the therapeutic composition e.g., an agent which effects the viscosity of the composition.

It should further be understood that the combinations which are to be included within this invention are those combinations useful for their intended purpose. The agents set forth below are illustrative for purposes and not intended to be limited. The combinations, which are part of this invention, can be the compounds of the present invention and at least one additional agent selected from the lists below. The combination can also include more than one additional agent, e.g., two or three additional agents if the combination is such that the formed composition can perform its intended function.

For example, in the treatment or prevention of inflammation, the present compounds may be used in conjunction or combination with an antiinflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent, for example with a compound such as acetaminophen, aspirin, codiene, fentanyl, ibuprofen, indomethacin, ketorolac, morphine, naproxen, phenacetin, piroxicam, a steroidal analgesic, sufentanyl, sunlindac, tenidap, and the like. Similarly, the instant compounds may be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedrine, or levo-desoxy-ephedrine; an antiitussive such as codeine, hydrocodone, caramiphen, carbetapentane, or dextromethorphan; a diuretic; and a sedating or non-sedating antihistamine. Likewise, compounds of the present invention may be used in combination with other drugs that are used in the treatment/prevention/suppression or amelioration of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention. Examples of other active ingredients that may be combined with a compound of the present invention, either administered separately or in the same pharmaceutical compositions, include, but are not limited to: (a) VLA-4 antagonists, (b) steroids such as beclomethasone, methylprednisolone, betamethasone, prednisone, dexamethasone, and hydrocortisone; (c) immunosuppressants such as cyclosporine (cyclosporine A, Sandimmune®, Neoral®), tacrolimus (FK-506, Prograf®), rapamycin (sirolimus, Rapamune®) and other FK-506 type immunosuppressants, and mycophenolate, e.g., mycophenolate mofetil (CellCept®); (d) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and the like; (e) non-steroidal anti-asthmatics such as .beta.2-agonists (terbutaline, metaproterenol, fenoterol, isoetharine, albuterol, bitolterol, and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (f) non- steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors such as celecoxib (Celebrex®) and rofecoxib (Vioxx®); (h) inhibitors of phosphodiesterase type IV (PDE-IV); (i) gold compounds such as auranofin and aurothioglucose, (j) inhibitors of phosphodiesterase type IV (PDE-IV); (k) other antagonists of the chemokine receptors, especially CCR1, CCR2, CCR3, CCR5, CCR6, CCR8 and CCR10; (l) cholesterol lowering agents such as HMG-CoA reductase inhibitors (lovastatin, simvastatin and pravastatin, fluvastatin, atorvastatin, and other statins), sequestrants (cholestyramine and colestipol), nicotinic acid, fenofibric acid derivatives (genfibrozil, clofibrat, fenofibrate and benzafibrate), and probucol; (m) anti-diabetic agents such as insulin, sulfonylureas, biguanides (metformin), α-glucosidase inhibitors (acarbose) and glitazones (troglitazone and pioglitazone); (n) preparations of interferon beta interferon β-1α; interferon β-1b;); (o) etanercept (Enbrel®), (p) antibody therapies such as orthoclone (OKT3), daclizumab (Zenapax®), infliximab (Remicade®), basiliximab (Simulect®) and anti-CD40 ligand antibodies (e.g., MRP-1); and (q) other compounds such as 5-aminosalicylic acid and prodrugs thereof, hydroxychloroquine, D- penicillamine, antimetabolites such as azathioprene and 6-mercaptopurine, and cytotoxic cancer chemotherapeutic agents. The weight ratio of the compound of the present invention to the second active ingredient may be varied and will depend upon the effective dose of each ingredient. Generally, an effective dose of each will be used. Thus, for example, when a compound of the present invention is combined with an NSAID the weight ratio of the compound of the present invention to the NSAID will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

Immunosuppressants within the scope of the present invention further include, but are not limited to, leflunomide, RAD001, ERL080, FTY720, CTLA4, antibody therapies such as orthoclone (OKT3), daclizumab (Zenapax®) and basiliximab (Simulect®), and antithymocyte globulins such as thymoglobulins.

In particularly preferred embodiments, the present methods are directed to the treatment or prevention of multiple sclerosis using a compound of the invention either alone or in combination with a second therapeutic agent selected from betaseron, avonex, azathioprene (Imurek®, Imuran®), capoxone, prednisolone and cyclophosphamide. When used in combination, the practitioner can administer a combination of the therapeutic agents, or administration can be sequential.

In still other particularly preferred embodiments, the present methods are directed to the treatment or prevention of rheumatoid arthritis, wherein the compound of the invention is administered either alone or in combination with a second therapeutic agent selected from the group consisting of methotrexate, sulfasalazine, hydroxychloroquine, cyclosporine A, D-penicillamine, infliximab (Remicade®), etanercept (Enbrel®), adalimumab (Humira®), auranofin and aurothioglucose.

In yet other particularly preferred embodiments, the present methods are directed to the treatment or prevention of an organ transplant condition wherein the compound of the invention is used alone or in combination with a second therapeutic agent selected from the group consisting of cyclosporine A, FK-506, rapamycin, mycophenolate, prednisolone, azathioprene, cyclophosphamide and an antilymphocyte globulin.

A compound of formula (I) of the invention may also be combined with agents, such as methotrexate, 6-MP, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signaling by proinflammatory cytokines such as TNFα or IL-1 (e.g. IRAK, NIK, IKK, p38 or MAP kinase inhibitors), IL-1β converting enzyme inhibitors, TNFα converting enzyme (TACE) inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors and the derivatives p75TNFRIgG (Enbrel™ and p55TNFRIgG (Lenercept)), sIL-1RI, sIL-1RII, sIL-6R), antiinflammatory cytokines (e.g. IL-4, IL-10, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone HCl, hydrocodone bitartrate/apap, diclofenac sodium/misoprostol, fentanyl, anakinra, human recombinant, tramadol HCl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline HCl, sulfadiazine, oxycodone HCl/acetaminophen, olopatadine HCl, misoprostol, naproxen sodium, omeprazole, cyclophosphamide, rituximab, IL-1 TRAP, MRA, CTLA4-IG, IL-18 BP, anti-IL-12, Anti-IL15, BIRB-796, SCIO-469, VX-702, AMG-548, VX-740, Roflumilast, IC-485, CDC-801, and Mesopram. Preferred combinations include methotrexate or leflunomide and in moderate or severe rheumatoid arthritis cases, cyclosporine and anti-TNF antibodies as noted above.

Non-limiting examples of therapeutic agents for inflammatory bowel disease with which a compound of formula (I) of the invention can be combined include the following: budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1β monoclonal antibodies; anti-IL-6 monoclonal antibodies; growth factors; elastase inhibitors; pyridinyl-imidazole compounds; antibodies to or antagonists of other human cytokines or growth factors, for example, TNF, LT, IL-1, IL-2, IL-6, IL-7, IL-8, IL-12, IL-15, IL-16, EMAP-II, GM-CSF, FGF, and PDGF; cell surface molecules such as CD2, CD3, CD4, CD8, CD25, CD28, CD30, CD40, CD45, CD69, CD90 or their ligands; methotrexate; cyclosporine; FK506; rapamycin; mycophenolate mofetil; leflunomide; NSAIDs, for example, ibuprofen; corticosteroids such as prednisolone; phosphodiesterase inhibitors; adenosine agonists; antithrombotic agents; complement inhibitors; adrenergic agents; agents which interfere with signalling by proinflammatory cytokines such as TNFα or IL-1 (e.g. IRAK, NIK, IKK, p38 or MAP kinase inhibitors); IL-1, converting enzyme inhibitors; TNFα converting enzyme inhibitors; T-cell signalling inhibitors such as kinase inhibitors; metalloproteinase inhibitors; sulfasalazine; azathioprine; 6-mercaptopurines; angiotensin converting enzyme inhibitors; soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-11, IL-13 and TGFβ). Preferred examples of therapeutic agents for Crohn's disease in which a compound of formula (I) can be combined include the following: TNF antagonists, for example, anti-TNF antibodies, D2E7 (U.S. Pat. No. 6,090,382; HUMIRA™), CA2 (REMICADE™), CDP 571, TNFR-Ig constructs, (p75TNFRIgG (ENBREL™) and p55TNFRIgG (LENERCEPT™)) inhibitors and PDE4 inhibitors. A compound of formula (I) can be combined with corticosteroids, for example, budenoside and dexamethasone; sulfasalazine, 5-aminosalicylic acid; olsalazine; and agents which interfere with synthesis or action of proinflammatory cytokines such as IL-1, for example, IL-1β converting enzyme inhibitors and IL-1ra; T cell signaling inhibitors, for example, tyrosine kinase inhibitors 6-mercaptopurines; IL-11; mesalamine; prednisone; azathioprine; mercaptopurine; infliximab; methylprednisolone sodium succinate; diphenoxylate/atrop sulfate; loperamide hydrochloride; methotrexate; omeprazole; folate; ciprofloxacin/dextrose-water; hydrocodone bitartrate/apap; tetracycline hydrochloride; fluocinonide; metronidazole; thimerosal/boric acid; cholestyramine/sucrose; ciprofloxacin hydrochloride; hyoscyamine sulfate; meperidine hydrochloride; midazolam hydrochloride; oxycodone HCl/acetaminophen; promethazine hydrochloride; sodium phosphate; sulfamethoxazole/trimethoprim; celecoxib; polycarbophil; propoxyphene napsylate; hydrocortisone; multivitamins; balsalazide disodium; codeine phosphate/apap; colesevelam HCl; cyanocobalamin; folic acid; levofloxacin; methylprednisolone; natalizumab and interferon-gamma.

Non-limiting examples of therapeutic agents for multiple sclerosis with which a compound of formula (I) can be combined include the following: corticosteroids; prednisolone; methylprednisolone; azathioprine; cyclophosphamide; cyclosporine; methotrexate; 4-aminopyridine; tizanidine; interferon-β1a (AVONEX; Biogen); interferon-β1b (BETASERON; Chiron/Berlex); interferon α-n3) (Interferon Sciences/Fujimoto), interferon-α (Alfa Wassermann/J&J), interferon β1A-IF (Serono/Inhale Therapeutics), Peginterferon α 2b (Enzon/Schering-Plough), Copolymer 1 (Cop-1; COPAXONE; Teva Pharmaceutical Industries, Inc.); hyperbaric oxygen; intravenous immunoglobulin; clabribine; antibodies to or antagonists of other human cytokines or growth factors and their receptors, for example, TNF, LT, IL-1, IL-2, IL-6, IL-7, IL-8, IL-12, IL-23, IL-15, IL-16, EMAP-II, GM-CSF, FGF, and PDGF. A compound of formula (I) can be combined with antibodies to cell surface molecules such as CD2, CD3, CD4, CD8, CD19, CD20, CD25, CD28, CD30, CD40, CD45, CD69, CD80, CD86, CD90 or their ligands. A compound of formula (I) may also be combined with agents, such as methotrexate, cyclosporine, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNFα or IL-1 (e.g. IRAK, NIK, IKK, p38 or MAP kinase inhibitors), IL-1β converting enzyme inhibitors, TACE inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-13 and TGFβ).

Preferred examples of therapeutic agents for multiple sclerosis in which a compound of formula (I) can be combined to include interferon-β, for example, IFNβ1a and IFNβ1b; copaxone, corticosteroids, caspase inhibitors, for example inhibitors of caspase-1, IL-1 inhibitors, TNF inhibitors, and antibodies to CD40 ligand and CD80.

A compound of formula (I) may also be combined with agents, such as alemtuzumab, dronabinol, Unimed, daclizumab, mitoxantrone, xaliproden hydrochloride, fampridine, glatiramer acetate, natalizumab, sinnabidol, α-immunokine NNSO3, ABR-215062, AnergiX.MS, chemokine receptor antagonists, BBR-2778, calagualine, CPI-1189, LEM (liposome encapsulated mitoxantrone), THC.CBD (cannabinoid agonist) MBP-8298, mesopram (PDE4 inhibitor), MNA-715, anti-IL-6 receptor antibody, neurovax, pirfenidone allotrap 1258 (RDP-1258), sTNF-R1, talampanel, teriflunomide, TGF-beta2, tiplimotide, VLA-4 antagonists (for example, TR-14035, VLA4 Ultrahaler, Antegran-ELAN/Biogen), interferon gamma antagonists and IL4 agonists.

Non-limiting examples of therapeutic agents for Angina with which a compound of formula (I) of the invention can be combined include the following: aspirin, nitroglycerin, isosorbide mononitrate, metoprolol succinate, atenolol, metoprolol tartrate, amlodipine besylate, diltiazem hydrochloride, isosorbide dinitrate, clopidogrel bisulfate, nifedipine, atorvastatin calcium, potassium chloride, furosemide, simvastatin, verapamil HCl, digoxin, propranolol hydrochloride, carvedilol, lisinopril, spironolactone, hydrochlorothiazide, enalapril maleate, nadolol, ramipril, enoxaparin sodium, heparin sodium, valsartan, sotalol hydrochloride, fenofibrate, ezetimibe, bumetanide, losartan potassium, lisinopril/hydrochlorothiazide, felodipine, captopril and bisoprolol fumarate.

Non-limiting examples of therapeutic agents for Ankylosing Spondylitis with which a compound of formula (I) can be combined include the following: ibuprofen, diclofenac and misoprostol, naproxen, meloxicam, indomethacin, diclofenac, celecoxib, rofecoxib, Sulfasalazine, Methotrexate, azathioprine, minocyclin, prednisone, etanercept, infliximab and adalimumab (Humira®).

Non-limiting examples of therapeutic agents for Asthma with which a compound of formula (I) can be combined include the following: albuterol, salmeterol/fluticasone, montelukast sodium, fluticasone propionate, budesonide, prednisone, salmeterol xinafoate, levalbuterol HCl, albuterol sulfate/ipratropium, prednisolone sodium phosphate, triamcinolone acetonide, beclomethasone dipropionate, ipratropium bromide, azithromycin, pirbuterol acetate, prednisolone, theophylline anhydrous, methylprednisolone sodium succinate, clarithromycin, zafirlukast, formoterol fumarate, influenza virus vaccine, methylprednisolone, amoxicillin trihydrate, flunisolide, allergy injection, cromolyn sodium, fexofenadine hydrochloride, flunisolide/menthol, amoxicillin/clavulanate, levofloxacin, inhaler assist device, guaifenesin, dexamethasone sodium phosphate, moxifloxacin HCl, doxycycline hyclate, guaifenesin/d-methorphan, p-ephedrine/cod/chlorphenir, gatifloxacin, cetirizine hydrochloride, mometasone furoate, salmeterol xinafoate, benzonatate, cephalexin, pe/hydrocodone/chlorphenir, cetirizine HCl/pseudoephed, phenylephrine/cod/promethazine, codeine/promethazine, cefprozil, dexamethasone, guaifenesin/pseudoephedrine, chlorpheniramine/hydrocodone, nedocromil sodium, terbutaline sulfate, epinephrine, methylprednisolone and metaproterenol sulfate.

Non-limiting examples of therapeutic agents for COPD with which a compound of formula (I) can be combined include the following: albuterol sulfate/ipratropium, ipratropium bromide, salmeterol/fluticasone, albuterol, salmeterol xinafoate, fluticasone propionate, prednisone, theophylline anhydrous, methylprednisolone sodium succinate, montelukast sodium, budesonide, formoterol fumarate, triamcinolone acetonide, levofloxacin, guaifenesin, azithromycin, beclomethasone dipropionate, levalbuterol HCl, flunisolide, ceftriaxone sodium, amoxicillin trihydrate, gatifloxacin, zafirlukast, amoxicillin/clavulanate, flunisolide/menthol, chlorpheniramine/hydrocodone, metaproterenol sulfate, methylprednisolone, mometasone furoate, p-ephedrine/cod/chlorphenir, pirbuterol acetate, p-ephedrine/loratadine, terbutaline sulfate, tiotropium bromide, (R,R)-formoterol, TgAAT, Cilomilast and Roflumilast.

Non-limiting examples of therapeutic agents for HCV with which a compound of formula (I) can be combined include the following: Interferon-alpha-2a, Interferon-alpha-2b, Interferon-alpha con1, Interferon-alpha-n1, Pegylated interferon-alpha-2a, Pegylated interferon-alpha-2b, ribavirin, Peginterferon alfa-2b+ribavirin, Ursodeoxycholic Acid, Glycyrrhizic Acid, Thymalfasin, Maxamine, VX-497 and any compounds that are used to treat HCV through intervention with the following targets: HCV polymerase, HCV protease, HCV helicase and HCV IRES (internal ribosome entry site).

Non-limiting examples of therapeutic agents for Idiopathic Pulmonary Fibrosis with which a compound of formula (I) can be combined include the following: prednisone, azathioprine, albuterol, colchicine, albuterol sulfate, digoxin, gamma interferon, methylprednisolone sod succ, lorazepam, furosemide, lisinopril, nitroglycerin, spironolactone, cyclophosphamide, ipratropium bromide, actinomycin d, alteplase, fluticasone propionate, levofloxacin, metaproterenol sulfate, morphine sulfate, oxycodone HCl, potassium chloride, triamcinolone acetonide, tacrolimus anhydrous, calcium, interferon-alpha, methotrexate, mycophenolate mofetil and Interferon-gamma-1β.

Non-limiting examples of therapeutic agents for Myocardial Infarction with which a compound of formula (I) can be combined include the following: aspirin, nitroglycerin, metoprolol tartrate, enoxaparin sodium, heparin sodium, clopidogrel bisulfate, carvedilol, atenolol, morphine sulfate, metoprolol succinate, warfarin sodium, lisinopril, isosorbide mononitrate, digoxin, furosemide, simvastatin, ramipril, tenecteplase, enalapril maleate, torsemide, retavase, losartan potassium, quinapril HCl/mag carb, bumetanide, alteplase, enalaprilat, amiodarone hydrochloride, tirofiban HCl m-hydrate, diltiazem hydrochloride, captopril, irbesartan, valsartan, propranolol hydrochloride, fosinopril sodium, lidocaine hydrochloride, eptifibatide, cefazolin sodium, atropine sulfate, aminocaproic acid, spironolactone, interferon, sotalol hydrochloride, potassium chloride, docusate sodium, dobutamine HCl, alprazolam, pravastatin sodium, atorvastatin calcium, midazolam hydrochloride, meperidine hydrochloride, isosorbide dinitrate, epinephrine, dopamine hydrochloride, bivalirudin, rosuvastatin, ezetimibe/simvastatin, avasimibe and cariporide.

Non-limiting examples of therapeutic agents for Psoriasis with which a compound of formula (I) can be combined include the following: calcipotriene, clobetasol propionate, triamcinolone acetonide, halobetasol propionate, tazarotene, methotrexate, fluocinonide, betamethasone diprop augmented, fluocinolone acetonide, acitretin, tar shampoo, betamethasone valerate, mometasone furoate, ketoconazole, pramoxine/fluocinolone, hydrocortisone valerate, flurandrenolide, urea, betamethasone, clobetasol propionate/emoll, fluticasone propionate, azithromycin, hydrocortisone, moisturizing formula, folic acid, desonide, pimecrolimus, coal tar, diflorasone diacetate, etanercept folate, lactic acid, methoxsalen, hc/bismuth subgal/znox/resor, methylprednisolone acetate, prednisone, sunscreen, halcinonide, salicylic acid, anthralin, clocortolone pivalate, coal extract, coal tar/salicylic acid, coal tar/salicylic acid/sulfur, desoximetasone, diazepam, emollient, fluocinonide/emollient, mineral oil/castor oil/na lact, mineral oil/peanut oil, petroleum/isopropyl myristate, psoralen, salicylic acid, soap/tribromsalan, thimerosal/boric acid, celecoxib, infliximab, cyclosporine, alefacept, efalizumab, tacrolimus, pimecrolimus, PUVA, UVB and sulfasalazine.

Non-limiting examples of therapeutic agents for Psoriatic Arthritis with which a compound of formula (I) can be combined include the following: methotrexate, etanercept, rofecoxib, celecoxib, folic acid, sulfasalazine, naproxen, leflunomide, methylprednisolone acetate, indomethacin, hydroxychloroquine sulfate, prednisone, sulindac, betamethasone diprop augmented, infliximab, methotrexate, folate, triamcinolone acetonide, diclofenac, dimethylsulfoxide, piroxicam, diclofenac sodium, ketoprofen, meloxicam, methylprednisolone, nabumetone, tolmetin sodium, calcipotriene, cyclosporine, diclofenac sodium/misoprostol, fluocinonide, glucosamine sulfate, gold sodium thiomalate, hydrocodone bitartrate/apap, ibuprofen, risedronate sodium, sulfadiazine, thioguanine, valdecoxib, alefacept, efalizumab and adalimumab (Humira®).

Non-limiting examples of therapeutic agents for Restenosis with which a compound of formula (I) can be combined include the following: sirolimus, paclitaxel, everolimus, tacrolimus, ABT-57 and acetaminophen.

Non-limiting examples of therapeutic agents for Sciatica with which a compound of formula (I) can be combined include the following: hydrocodone bitartrate/apap, rofecoxib, cyclobenzaprine HCl, methylprednisolone, naproxen, ibuprofen, oxycodone HCl/acetaminophen, celecoxib, valdecoxib, methylprednisolone acetate, prednisone, codeine phosphate/apap, tramadol HCl/acetaminophen, metaxalone, meloxicam, methocarbamol, lidocaine hydrochloride, diclofenac sodium, gabapentin, dexamethasone, carisoprodol, ketorolac tromethamine, indomethacin, acetaminophen, diazepam, nabumetone, oxycodone HCl, tizanidine HCl, diclofenac sodium/misoprostol, propoxyphene napsylate/apap, asa/oxycod/oxycodone ter, ibuprofen/hydrocodone bit, tramadol HCl, etodolac, propoxyphene HCl, amitriptyline HCl, carisoprodol/codeine phos/asa, morphine sulfate, multivitamins, naproxen sodium, orphenadrine citrate and temazepam.

Preferred examples of therapeutic agents for SLE (Lupus) in which a compound of formula (I) include the following: NSAIDS, for example, diclofenac, naproxen, ibuprofen, piroxicam, indomethacin; COX2 inhibitors, for example, Celecoxib, rofecoxib, valdecoxib; anti-malarials, for example, hydroxychloroquine; Steroids, for example, prednisone, prednisolone, budenoside, dexamethasone; Cytotoxics, for example, azathioprine, cyclophosphamide, mycophenolate mofetil, methotrexate; inhibitors of PDE4 or purine synthesis inhibitor, for example Cellcept. A compound of formula (I) may also be combined with agents such as sulfasalazine, 5-aminosalicylic acid, olsalazine, Imuran and agents which interfere with synthesis, production or action of proinflammatory cytokines such as IL-1, for example, caspase inhibitors like IL-1β converting enzyme inhibitors and IL-Ira. A compound of formula (I) may also be used with T cell signaling inhibitors, for example, tyrosine kinase inhibitors; or molecules that target T cell activation molecules, for example, CTLA4-IgG or anti-B7 family antibodies, anti-PD-1 family antibodies. A compound of formula (I) can be combined with IL-11 or anti-cytokine antibodies, for example, fonotolizumab (anti-IFNg antibody), or anti-receptor receptor antibodies, for example, anti-IL-6 receptor antibody and antibodies to B-cell surface molecules. A compound of formula (I) may also be used with LJP 394 (abetimus), agents that deplete or inactivate B-cells, for example, Rituximab (anti-CD20 antibody), lymphostat-B (anti-BlyS antibody), TNF antagonists, for example, anti-TNF antibodies, adalimumab (HUMIRA™), CA2 (REMICADE™), CDP 571, TNFR-Ig constructs, (p75TNFRIgG (ENBREL™) and p55TNFRIgG (LENERCEPT™)).

In this invention, the following definitions are applicable:

“Pharmaceutically acceptable salts” refers to those salts which retain the biological effectiveness and properties of the free bases and which are obtained by reaction with inorganic acids, for example, hydrochloric acid, hydrobromic acid, hydroiodic acid, sulfuric acid, nitric acid, and phosphoric acid or organic acids such as sulfonic acid, carboxylic acid, organic phosphoric acid, methanesulfonic acid, ethanesulfonic acid, p-toluenesulfonic acid, citric acid, fumaric acid, maleic acid, succinic acid, benzoic acid, salicylic acid, lactic acid, acetic acid, trifluoracetic acid, tartaric acid (e.g. (+) or (−)-tartaric acid or mixtures thereof), amino acids (e.g. (+) or (−)-amino acids or mixtures thereof), and the like. The compounds of this invention embrace radicals having “cis” and “trans” orientations, or alternatively, “E” and “Z” orientations, as appreciated by those of ordinary skill in the art. These salts can be prepared by methods known to those skilled in the art.

Certain compounds of formula I which have acidic substituents may exist as salts with pharmaceutically acceptable salts with bases. The present invention includes such salts. Examples of such salts include sodium salts, potassium salts, lysine salts and arginine salts. These salts may be prepared by methods known to those skilled in the art.

Certain compounds of formula I and their salts may exist in more than one crystal form and the present invention includes each crystal form and mixtures thereof.

Certain compounds of formula I and their salts may also exist in the form of solvates, for example hydrates, and the present invention includes each solvate and mixtures thereof.

Certain compounds of formula I may contain one or more chiral centers, and exist in different optically active forms. When compounds of formula I contain one chiral center, the compounds exist in two enantiomeric forms and the present invention includes both enantiomers and mixtures of enantiomers, such as racemic mixtures. The enantiomers may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a compound of formula I contains more than one chiral center it may exist in diastereoisomeric forms. The diastereoisomeric pairs may be separated by methods known to those skilled in the art, for example chromatography or crystallization and the individual enantiomers within each pair may be separated as described above. The present invention includes each diastereoisomer of compounds of formula I and mixtures thereof.

Certain compounds of formula I may exist in different tautomeric forms or as different geometric isomers, and the present invention includes each tautomer and/or geometric isomer of compounds of formula I and mixtures thereof.

Certain compounds of formula I may exist in different stable conformational forms which may be separable. Torsional asymmetry due to restricted rotation about an asymmetric single bond, for example because of steric hindrance or ring strain, may permit separation of different conformers. The present invention includes each conformational isomer of compounds of formula I and mixtures thereof.

Certain compounds of formula I may exist in zwitterionic form and the present invention includes each zwitterionic form of compounds of formula I and mixtures thereof.

As used herein the term “pro-drug” refers to an agent which is converted into the parent drug in vivo by some physiological chemical process (e.g., a prodrug on being brought to the physiological pH is converted to the desired drug form). Pro-drugs are often useful because, in some situations, they may be easier to administer than the parent drug. They may, for instance, be bioavailable by oral administration whereas the parent drug is not. The prodrug may also have improved solubility in pharmacological compositions over the parent drug. An example, without limitation, of a pro-drug would be a compound of the present invention wherein it is administered as an ester (the “pro-drug”) to facilitate transmittal across a cell membrane where water solubility is not beneficial, but then it is metabolically hydrolyzed to the carboxylic acid once inside the cell where water solubility is beneficial

Pro-drugs have many useful properties. For example, a pro-drug may be more water soluble than the ultimate drug, thereby facilitating intravenous administration of the drug. A pro-drug may also have a higher level of oral bioavailability than the ultimate drug. After administration, the prodrug is enzymatically or chemically cleaved to deliver the ultimate drug in the blood or tissue.

Exemplary pro-drugs upon cleavage release the corresponding free acid, and such hydrolyzable ester-forming residues of the compounds of this invention include but are not limited to carboxylic acid substituents (e.g., —(CH₂)C(O)H or a moiety that contains a carboxylic acid) wherein the free hydrogen is replaced by (C₁-C₄)alkyl, (C₂-C₁₂)alkanoyloxymethyl, (C₄-C₉)1-(alkanoyloxy)ethyl, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)-alkylcarbamoyl-(C₁-C₂)alkyl and piperidino-, pyrrolidino- or morpholino(C₂-C₃)alkyl.

Other exemplary pro-drugs release an alcohol of Formula I wherein the free hydrogen of the hydroxyl substituent (e.g., R¹ contains hydroxyl) is replaced by (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylamino-methyl, succinoyl, (C₁-C₆)alkanoyl, α-amino(C₁-C₄)alkanoyl, arylactyl and α-aminoacyl, or α-aminoacyl-α-aminoacyl wherein said α-aminoacyl moieties are independently any of the naturally occurring L-amino acids found in proteins, P(O)(OH)₂, —P(O)(O(C₁-C₆)alkyl)₂ or glycosyl (the radical resulting from detachment of the hydroxyl of the hemiacetal of a carbohydrate).

The term “heterocyclic” or “heterocyclyl” as used herein, include non-aromatic, ring systems, including, but not limited to, monocyclic, bicyclic and tricyclic rings, which can be completely saturated or which can contain one or more units of unsaturation and have 3 to 12 atoms including at least one heteroatom, such as nitrogen, oxygen, or sulfur. For purposes of exemplification, which should not be construed as limiting the scope of this invention: azetidinyls, morpholines, pyrrolidines, piperazines, piperidines, pyrans, triazoles, tetrazoles, tetrahydropyranyl, thiadiazoles, thiomorpholines or triazoles.

The term “heteroaryl”, as used herein include aromatic and non-aromatic, ring systems, including, but not limited to, monocyclic, bicyclic and tricyclic rings, which can be completely saturated or which can contain one or more units of unsaturation and have 3 to 12 atoms including at least one heteroatom, such as nitrogen, oxygen, or sulfur. For purposes of exemplification, which should not be construed as limiting the scope of this invention: azaindole, benzo[b]thienyl, benzimidazolyl, benzo[1,3]dioxolyl, benzo[1,3]dioxazinyl, benz[1,3,4]oxathiazinyl, dihydrobenz[1,4]oxazinyl, benzo[1,4]oxazinyl, benzo[d]isoxazolyl, benzo[d]isothiazolyl, benzofuranyl, benzotriazolyl, benzoxazolyl, benzothiazolyl, benzothiadiazolyl, benzo[1,2,5]isoxazolyl, benzo[1,2,5]oxadiazolyl, benzo[1,2,5]thiadiazolyl, benzoxadiazolyl, cinnolines, chromenes, dihydrobenzofurans, 2,3-dihydrobenzo[1,4]dioxines, 2,3-dihydrobenzo[1,4]dioxines, 2,3-dihydrobenzo[b]thiophenes, 5,6-dihydroimidazo[2,1-b]thiazoles, dihydroindazoles, dihydroquinolines, furans, furazans, imidazoles, imidazoles, imidazopyridine, imidazo[1,2-a]pyridines, imidazo[1,2-b]pyridazines, imidazo[2,1-b]thiazoles, indoles, indans, indazoles, isoxazoles, isoquinolines, isothiazoles, isoxazoles, oxadiazoles, oxazoles, naphthyridines, purine, pyrazines, pyrazoles, pyridazines pyridines, pyrimidines, pyrroles, pyrrolidines, pyrrolo[2,3-d]pyrimidine, pyrazolo[1,5-a]pyrimidinyl, pyrazolo[3,4-b]pyrimidine, pyrazolo[3,4-d]pyrimidine), quinazolines, quinoxalines, quinolines, quinazolines, thiazoles, [1,2,4]triazolyl, [1,2,4]triazolo[1,5-a]pyrimidinyl, 4,5,6,7-tetrahydroindazoles, tetrahydronaphthyl, tetrahydroquinolines, tetrahydroisoquinolines, tetrahydroindole, thieno[2,3-d]pyrimidines, or thienyls.

As used herein, many moieties or substituents are termed as being either “substituted” or “optionally substituted”. When a moiety is modified by one of these terms, it denotes that any portion of the moiety that is known to one skilled in the art as being available for substitution can be substituted, which includes one or more substituents, where if more than one substituent then each substituent is independently selected. Such means for substitution are well-known in the art and/or taught by the instant disclosure. For purposes of exemplification, which should not be construed as limiting the scope of this invention, some examples of groups that are substituents are: alkenyl groups, alkoxy group (which itself can be substituted, such as —O—C₁-C₆-alkyl-OR, —O—C₁-C₆-alkyl-N(R)₂, and OCF₃), alkoxyalkoxy, alkoxycarbonyl, alkoxycarbonylpiperidinyl-alkoxy, alkyl groups (which itself can also be substituted, such as —C₁-C₆-alkyl-OR, —C₁-C₆-alkyl-N(R)₂, and —CF₃), alkylamino, alkylcarbonyl, alkylester, alkylnitrile, alkylsulfonyl, amino, aminoalkoxy, benzyl, CF₃, COH, COOH, CN, cycloalkyl, dialkylamino, dialkylaminoalkoxy, dialkylaminocarbonyl, dialkylaminocarbonylalkoxy, dialkylaminosulfonyl, esters (—C(O)—OR, where R is groups such as alkyl, heterocycloalkyl (which can be substituted), heterocyclyl, etc., which can be substituted), halogen or halo group (F, Cl, Br, I), hydroxy, morpholinoalkoxy, morpholinoalkyl, nitro, oxo, OCF₃, optionally substituted phenyl, S(O)₂N(CH₃)₂, S(O)₂CH₃, S(O)₂CF₃, and sulfonyl, N-alkylamino or N,N-dialkylamino (in which the alkyl groups can also be substituted).

For purposes of exemplification, which should not be construed as limiting the scope of this invention, some examples of groups that are substituents of amine groups are: alkenyl groups, alkyl groups (which itself can also be substituted, such as —C₁-C₆-alkyl-OR, —C₁-C₆-alkyl-N(R)₂, and —CF₃), —C(O)—O-alkyl, cycloalkyl, phenylcarbonyl (which itself can also be substituted) 1, benzylcarbonyl (which itself can also be substituted), thienylcarbonyl (which itself can also be substituted) and alkylcarbonyl (which itself can also be substituted), benzyl (which itself can also be substituted) and phenyl (which itself can also be substituted).

When the term “substituted heterocyclic” (or heterocyclyl), “substituted heteroaryl (or heteroaryl) or “substituted aryl” (or aryl) is used, what is meant is that the heterocyclic group is substituted with one or more substituents that can be made by one of ordinary skill in the art and results in a molecule that is a kinase inhibitor. For purposes of exemplification, which should not be construed as limiting the scope of this invention, preferred substituents for the heterocyclyls of this invention are each independently selected from the optionally substituted group consisting of alkenyl, alkoxy, alkoxyalkoxy, alkoxyalkyl, alkoxycarbonyl, alkoxycarbonylheterocycloalkoxy, alkyl, alkylcarbonyl, alkylester, alkyl-O—C(O)—, alkyl-heterocyclyl, alkyl-cycloalkyl, alkyl-cycloalkenyl, alkyl-nitrile, alkynyl, amido groups, amino, aminoalkyl, aminocarbonyl, benzyl, carbonitrile, carbonylalkoxy, carboxamido, CF₃, CN, —C(O)OH, —C(O)H, —C(O)—(O)(CH₃)₃, —OH, —C(O)O-alkyl, —C(O)O-cycloalkyl, —C(O)O-heterocyclyl, —C(O)-alkyl, —C(O)-amino, —C(O)-cycloalkyl, —C(O)-heterocyclyl, —C(O)—NH—R_{c}, cycloalkyl, dialkylaminoalkoxy, dialkylaminocarbonylalkoxy, dialkylaminocarbonyl, halogen, heterocyclyl, a heterocycloalkyl group, heterocyclyloxy, hydroxy, hydroxyalkyl, morpholinyl, nitro, NO₂, OCF₃, oxo, phenyl, phenylcarbonyl, pyrrolidinyl, —SO₂CH₃, —SO₂CR₃, tetrazolyl, thienylalkoxy, trifluoromethylcarbonylamino, trifluoromethylsulfonamido, heterocyclylalkoxy, heterocyclyl-S(O)ₚ, cycloalkyl-S(O)ₚ, alkyl-S—, heterocyclyl-S, heterocycloalkyl, cycloalkylalkyl, heterocycolthio, cycloalkylthio, -Z¹⁰⁵-C(O)N(R)₂, -Z¹⁰⁵-N(R)—C(O)-Z²⁰⁰, -Z¹⁰⁵-N(R)—S(O)₂-Z²⁰⁰, -Z¹⁰⁵-N(R)—C(O)—N(R)-Z²⁰⁰, —N(R)—C(O)R, —N(R)—C(O)OR, OR—C(O)-heterocyclyl-OR, R_{c} and —CH₂OR_{c};
where R_{c} for each occurrence is independently hydrogen, optionally substituted alkyl, optionally substituted aryl, —(C₁-C₆)—NR_{d}Rₑ, -E-(CH₂)ₜ—NR_{d}Rₑ, -E-(CH₂)ₜ—O-alkyl, -E-(CH₂), —S-alkyl, or -E-(CH₂)ₜ—OH
   wherein t is an integer from about 1 to about 6;
Z¹⁰⁵ for each occurrence is independently a covalent bond, alkyl, alkenyl or alkynyl; and
Z²⁰⁰ for each occurrence is independently selected from an optionally substituted group selected from the group consisting of alkyl, alkenyl, alkynyl, phenyl, alkyl-phenyl, alkenyl-phenyl or alkynyl-phenyl;
E is a direct bond, O, S, S(O), S(O)₂, or NR_{f}, wherein R_{f} is H or alkyl and R_{d} and Rₑ are independently H, alkyl, alkanoyl or SO₂-alkyl; or R_{d}, Rₑ and the nitrogen atom to which they are attached together form a five- or six-membered heterocyclic ring.

When the term “substituted phenyl” is used, what is meant is that the phenyl group is substituted with one or more substituents that can be made by one of ordinary skill in the art and results in a molecule that is a kinase inhibitor. For purposes of exemplification, which should not be construed as limiting the scope of this invention, preferred substituents for the phenyls of this invention are each independently selected from the optionally substituted group consisting of alkenyl, alkoxy, alkoxyalkyl, alkoxycarbonyl, alkyl, alkylcarbonyl, alkylester, alkyl-heterocyclyl, alkyl-cycloalkyl, alkyl-cycloalkenyl, alkynyl, amido groups, amino, aminoalkyl, aminocarbonyl, benzyl, carbonitrile, carbonylalkoxy, CF₃, CHF₂, CN, —C(O)OH, —C(O)H, —C(O)—(O)(CH₃)₃, —OH, —C(O)-alkyl, —C(O)-amino, —C(O)-cycloalkyl, —C(O)-heterocyclyl, —C(O)—NH-heterocyclyl, especially —C(O)—NH-tetrazolyl, cycloalkyl, dialkylaminoalkoxy, dialkylaminocarbonyl, halogen, heterocyclyl, a heterocycloalkyl group, heterocyclyloxy, hydroxy, hydroxyalkyl, morpholinyl, nitro, NO₂, OCF₃, oxo, phenyl, pyrrolidinyl, —SO₂CH₃, —SO₂CR₃, tetrazolyl, trifluoromethylsulfonamido, heterocyclylalkoxy, heterocyclyl-S(O)ₚ, cycloalkyl-S(O)ₚ, alkyl-S—, heterocyclyl-S, heterocycloalkyl, cycloalkylalkyl, heterocycolthio, cycloalkylthio, -Z¹⁰⁵-C(O)N(R)₂, -Z¹⁰⁵-N(R)—C(O)-Z²⁰⁰, -Z¹⁰⁵—N(R)—S(O)₂-Z²⁰⁰, -Z¹⁰⁵-N(R)—C(O)—N(R)-Z²⁰⁰, —N(R)—C(O)R, —N(R)—C(O)OR, OR—C(O)-heterocyclyl-OR, R_{c} and —CH₂OR_{c};
where R_{c} for each occurrence is independently hydrogen, optionally substituted alkyl, optionally substituted aryl, —(C₁-C₆)—NR_{d}Rₑ, -E-(CH₂)ₜ—NR_{d}Rₑ, -E-(CH₂)ₜ—O-alkyl, -E-(CH₂)ₜ—S-alkyl, or -E-(CH₂)ₜ—OH
   wherein t is an integer from about 1 to about 6;
Z¹⁰⁵ for each occurrence is independently a covalent bond, alkyl, alkenyl or alkynyl; and
Z²⁰⁰ for each occurrence is independently selected from an optionally substituted group selected from the group consisting of alkyl, alkenyl, alkynyl, phenyl, alkyl-phenyl, alkenyl-phenyl or alkynyl-phenyl;
E is a direct bond, O, S, S(O), S(O)₂, or NR_{f}, wherein R_{f} is H or alkyl and R_{d} and Rₑ are independently H, alkyl, alkanoyl or SO₂-alkyl; or R_{d}, Rₑ and the nitrogen atom to which they are attached together form a five- or six-membered heterocyclic ring.

An “heterocycloalkyl” group, as used herein, is a heterocyclic group that is linked to a compound by an aliphatic group having from one to about eight carbon atoms. For example, imidazolylethyl, tetrahydropyranylmethyl, morpholinoethyl, morpholinomethyl, piperidinylmethyl and pyrrolidinylmethyl groups are examples of heterocycloalkyl groups.

As used herein, “aliphatic” or “an aliphatic group” or notations such as “(C₀-C₈)” include straight chained or branched hydrocarbons which are completely saturated or which contain one or more units of unsaturation, and, thus, includes alkyl, alkenyl, alkynyl and hydrocarbons comprising a mixture of single, double and triple bonds. When the group is a C₀ it means that the moiety is not present or in other words, it is a bond. As used herein, “alkyl” means C₁-C₈ and includes straight chained or branched hydrocarbons which are completely saturated. Preferred alkyls are methyl, ethyl, propyl, butyl, pentyl, hexyl, isopropyl, isobutyl, t-butyl, tert-butyl, and isomers thereof. As used herein, “alkenyl” and “alkynyl” means C₂-C₈ and includes straight chained or branched hydrocarbons which contain one or more units of unsaturation, one or more double bonds for alkenyl and one or more triple bonds for alkynyl.

As used herein “alkylidenyl” means C₁-C₄ bivalent radicals derived from saturated unbranched alkanes by removal of two hydrogen atoms, for example, —CH₂—, —CH₂CH₂—, —CH₂—CH₂—CH₂—, —CH₂CH₂—CH₂CH₂—.

As used herein, aromatic groups (or aryl groups) include aromatic carbocyclic ring systems (e.g. phenyl and cyclopentyldienyl) and fused polycyclic aromatic ring systems (e.g. naphthyl and quinolinyl).

As used herein, cycloalkyl means C₃-C₁₂ monocyclic or multicyclic (e.g., bicyclic, tricyclic, etc.) hydrocarbons which is completely saturated or has one or more unsaturated bonds but does not amount to an aromatic group. Preferred examples of a cycloalkyl group are adamantanyl, cyclopropyl, cyclobutyl, cyclopentyl, cyclopentenyl, cyclohexyl and cyclohexenyl.

As used herein, amido group means —NHC(═O)—.

As used herein, acyloxy groups are —OC(O)R.

The term “alkoxyalkyl”, as used herein includes, but is not limited to, moieties such as —CH₂—OCH₃—CH(CH₃)—OEt and —CH₂—OEt-OCH₃.

The term “alkoxyalkoxyalkyl”, as used herein includes, but is not limited to, moieties such as —CH₂—OEt-OEt and —CH₂—OEt-OEt-OEt.

The term “aryl”, as used herein includes, but is not limited to, moieties such as fluorene, naphthyl, tetrahydronaphthyl and phenyl.

The term “diphenylalkyl”, as used herein includes, but is not limited to, moieties such as methyl disubstituted with phenyl.

The term “alkoxy”, as used herein includes, but is not limited to, moieties such as OCH₃.

The term “arylalkyl”, as used herein includes, but is not limited to, moieties such as benzyl and phenylethyl.

The term “cycloalkylalkyl”, as used herein includes, but is not limited to, moieties such as cyclopropylmethyl, cyclohexylmethyl and cyclopentylmethyl.

The term “aminoalkyl”, as used herein includes, but is not limited to, moieties such as aminopropyl.

The term “aminoalkylamine”, as used herein includes, but is not limited to, moieties such as diethylaminoethylamine.

The term “aryloxy”, as used herein includes, but is not limited to, moieties such as benzyloxy and phenoxy.

The term “cycloalkyenyl”, as used herein includes, but is not limited to, moieties such as cyclobutene.

The term “aryloxyalkyl”, as used herein includes, but is not limited to, moieties such as benzyloxy.

The term “aralkyl”, as used herein includes, but is not limited to, moieties such as benzyl.

Pharmaceutical Formulations

One or more compounds of this invention can be administered to a human patient by themselves or in pharmaceutical compositions where they are mixed with biologically suitable carriers or excipient(s) at doses to treat or ameliorate a disease or condition as described herein. Mixtures of these compounds can also be administered to the patient as a simple mixture or in suitable formulated pharmaceutical compositions. A therapeutically effective dose refers to that amount of the compound or compounds sufficient to result in the prevention or attenuation of a disease or condition as described herein. Techniques for formulation and administration of the compounds of the instant application may be found in references well known to one of ordinary skill in the art, such as “Remington's Pharmaceutical Sciences,” Mack Publishing Co., Easton, Pa., latest edition.

Routes of Administration.

Suitable routes of administration may, for example, include oral, eyedrop, rectal, transmucosal, topical, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one may administer the compound in a local rather than a systemic manner, for example, via injection of the compound directly into an edematous site, often in a depot or sustained release formulation.

Furthermore, one may administer the drug in a targeted drug delivery system, for example, in a liposome coated with endothelial cell-specific antibody.

Composition/Formulation

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds can be formulated for parenteral administration by injection, e.g. bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly or by intramuscular injection). Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

An example of a pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethysulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free base forms.

Effective Dosage

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art.

For any compound used in a method of the present invention, the therapeutically effective dose can be estimated initially from cellular assays. For example, a dose can be formulated in cellular and animal models to achieve a circulating concentration range that includes the IC₅₀ as determined in cellular assays (i.e., the concentration of the test compound which achieves a half-maximal inhibition of a given protein kinase activity). In some cases it is appropriate to determine the IC₅₀ in the presence of 3 to 5% serum albumin since such a determination approximates the binding effects of plasma protein on the compound. Such information can be used to more accurately determine useful doses in humans. Further, the most preferred compounds for systemic administration effectively inhibit protein kinase signaling in intact cells at levels that are safely achievable in plasma.

A therapeutically effective dose refers to that amount of a compound of Formula I or a combination of two or more such compounds, which inhibits, totally or partially, the progression of a condition or alleviates, at least partially, one or more symptoms of the condition. A therapeutically effective amount can also be an amount which is prophylactically effective. Toxicity and therapeutic efficacy of such compounds can be determined by standard pharmaceutical procedures in cell cultures or experimental animals, e.g., for determining the maximum tolerated dose (MTD) and the ED₅₀ (effective dose for 50% maximal response). The dose ratio between toxic and therapeutic effects is the therapeutic index and it can be expressed as the ratio between MTD and ED₅₀. Compounds which exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. A therapeutically effective amount can also be an amount which is prophylactically effective. The amount which is therapeutically effective will depend upon the patient's size and gender, the condition to be treated, the severity of the condition and the result sought. For a given patient, a therapeutically effective amount can be determined by methods known to those of skill in the art. The exact formulation, route of administration and dosage can be chosen by the individual physician in view of the patient's condition. (See e.g. Fingl et al., 1975, in “The Pharmacological Basis of Therapeutics”, Ch. 1 p1). In the treatment of crises, the administration of an acute bolus or an infusion approaching the MTD may be required to obtain a rapid response.

Dosage amount and interval may be adjusted individually to provide plasma levels of the active moiety which are sufficient to maintain the kinase modulating effects, or minimal effective concentration (MEC). The MEC will vary for each compound but can be estimated from in vitro data; e.g. the concentration necessary to achieve 50-90% inhibition of protein kinase using the assays described herein. Dosages necessary to achieve the MEC will depend on individual characteristics and route of administration. However, HPLC assays or bioassays can be used to determine plasma concentrations.

Dosage intervals can also be determined using the MEC value. Compounds should be administered using a regimen which maintains plasma levels above the MEC for 10-90% of the time, preferably between 30-90% and most preferably between 50-90% until the desired amelioration of symptoms is achieved. In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

Packaging

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labelled for treatment of an indicated condition.

In some formulations it may be beneficial to use the compounds of the present invention in the form of particles of very small size, for example as obtained by fluid energy milling.

The use of compounds of the present invention in the manufacture of pharmaceutical compositions is illustrated by the following description. In this description the term “active compound” denotes any compound of the invention but particularly any compound which is the final product of one of the preceding Examples.

a) Capsules

In the preparation of capsules, 10 parts by weight of active compound and 240 parts by weight of lactose can be de-aggregated and blended. The mixture can be filled into hard gelatin capsules, each capsule containing a unit dose or part of a unit dose of active compound.

b) Tablets

Tablets can be prepared, for example, from the following ingredients.

Parts by weight

| | |
|---|---|
| Active compound | 10 |
| Lactose | 190 |
| Maize starch | 22 |
| Polyvinylpyrrolidone | 10 |
| Magnesium stearate | 3 |

The active compound, the lactose and some of the starch can be de-aggregated, blended and the resulting mixture can be granulated with a solution of the polyvinyl-pyrrolidone in ethanol. The dry granulate can be blended with the magnesium stearate and the rest of the starch. The mixture is then compressed in a tabletting machine to give tablets each containing a unit dose or a part of a unit dose of active compound.

c) Enteric Coated Tablets

Tablets can be prepared by the method described in (b) above. The tablets can be enteric coated in a conventional manner using a solution of 20% cellulose acetate phthalate and 3% diethyl phthalate in ethanol:dichloromethane (1:1).

d) Suppositories

In the preparation of suppositories, for example, 100 parts by weight of active compound can be incorporated in 1300 parts by weight of triglyceride suppository base and the mixture formed into suppositories each containing a therapeutically effective amount of active ingredient.

In the compositions of the present invention the active compound may, if desired, be associated with other compatible pharmacologically active ingredients. For example, the compounds of this invention can be administered in combination with another therapeutic agent that is known to treat a disease or condition described herein. For example, with one or more additional pharmaceutical agents that inhibit or prevent the production of VEGF or angiopoietins, attenuate intracellular responses to VEGF or angiopoietins, block intracellular signal transduction, inhibit vascular hyperpermeability, reduce inflammation, or inhibit or prevent the formation of edema or neovascularization. The compounds of the invention can be administered prior to, subsequent to or simultaneously with the additional pharmaceutical agent, whichever course of administration is appropriate. The additional pharmaceutical agents include, but are not limited to any of the agents, for examples, described in pages 20-28. The compounds of the invention and the additional pharmaceutical agents act either additively or synergistically. Thus, the administration of such a combination of substances that inhibit angiogenesis, vascular hyperpermeability and/or inhibit the formation of edema can provide greater relief from the deletrious effects of a hyperproliferative disorder, angiogenesis, vascular hyperpermeability or edema than the administration of either substance alone. In the treatment of malignant disorders combinations with antiproliferative or cytotoxic chemotherapies or radiation are included in the scope of the present invention.

The present invention also comprises the use of a compound of formula I as a medicament.

A further aspect of the present invention provides the use of a compound of formula I or a salt thereof in the manufacture of a medicament for treating vascular hyperpermeability, angiogenesis-dependent disorders, proliferative diseases and/or disorders of the immune system in mammals, particularly human beings.

The present invention also provides a method of treating vascular hyperpermeability, inappropriate neovascularization, proliferative diseases and/or disorders of the immune system which comprises the administration of a therapeutically effective amount of a compound of formula I to a mammal, particularly a human being, in need thereof.

The contents of all references, patents and published patent applications, in their entirety, cited throughout this application are incorporated herein by reference.

Assays for Screening Compounds of Formula (I)

The in vitro potency of compounds in inhibiting P2X7 is discussed herein or described in the art may be determined by the procedures detailed below.

FLIPR Assay

Tissue culture. 1321N1 human astrocytoma cells stably expressing the following recombinant receptors were cloned, transfected and grown according to previously published protocols (Bianchi et al, 1999; Lynch et al., 1999). Briefly, human, rat, and mouse P2X₇ cells were maintained in a humidified 5% CO₂ atmosphere at 37 C in DMEM containing 1% L-Alanyl-L-Glutamine, 1% Antibiotic/Antimycotic, 10% FBS and 300 ug/ml Geneticin. Ca²⁺ Influx FLIPR Assay. Agonist-induced Ca²⁺ dynamics were assessed in all of the cell lines using the Ca²⁺ chelating dye, Fluo-4, in conjunction with a Fluorometric Imaging Plate Reader (FLIPR; Molecular Devices, Sunnyvale, Calif.) as previously described (Bianchi et al, 1999) with noted minor modifications. The cells were plated out the day before the experiment onto Poly-D-Lysine coated black 96 well plates (Becton-Dickinson, Bedford, Mass. and Sigma, St. Louis Mo.). Cell concentration was 5×10⁶ cells per plate. Fluo-4 was dissolved in anhydrous DMSO to a final concentration of 5 ug/ml in DPBS. The dye was loaded onto the adherent cells and the plates were centrifuged for 5 minutes at 1000 rpm. Cells were loaded for at least one hour, but not more than 3 hours and kept in the dark at room temperature. After loading, the unincorporated Fluo-4 was removed by washing with DPBS using a SkanWasher 400 (Molecular Devices, Sunnyvale, Calif.). All compound solutions were prepared in DPBS. After the agonist addition, Ca²⁺ dynamics were recorded on a second time scale for 3 minutes using the FLIPR. Ligands were tested at 11 half-log concentrations. Independent measurements of a positive control (100%) were performed on each plate in order to normalize values from plate to plate. 5 uM BzATP was used as positive control for human P2X₇, 10 uM BzATP for rat P2X₇ and 150 uM BzATP for mouse P2X₇. For measurement of antagonist activity, ligands were added to the cell plate and fluorescence data collected for 3 minutes before the addition of the agonist. Fluorescence data was collected for another 2 minutes after the agonist addition. Controls used for normalization in the antagonist experiments were the same as those used for agonist experiments. Concentration-response data were analyzed using non-linear regression in Assay Explorer, the IC₅₀ values were derived from a single curve fit to the mean data of n=4-6, in duplicates.

Human Whole Blood Assay Protocol:

1. Obtain fresh blood in heparinized (green-top, 10 ml vacutainer) tubes.
2. Mix 25 ml blood from each donor with 25 ml RPMI/20 mM HEPES.
3. Add 150 ul/well of blood/RPMI to wells in columns 1-10 of a 96 well flat bottomed plate (Costar # 3599).
4. Compound Dilution: Done in 96 well, round bottom polypropylene plates.
   a. Take 10 mM stock (100% DMSO) and dilute 1:5 (10 ul+40 ul DMSO) in row
   A (one compound/column)=2 mM in 100% DMSO.
   b. Add 40 ul DMSO to wells in rows B-H. Make 1:5 serial dilutions of the 2 mM compounds (10 ul+40 ul DMSO) down to row F. Rows G and H are no drug treatment wells.
   c. Dilute the dilutions 1:4 in RPMI+20 mM HEPES (10 ul+30 ul medium)=25× compound in 25% DMSO in a new plate, making duplicate columns for each drug (5 drugs/plate).
   d. Transfer 6 ul/well of each dilution into blood plate wells. This is a 1:25 dilution that will give 1× compound in 1% DMSO final concentration Add 6 ul 25% DMSO to control wells.
5. Incubate at 37°/5% CO₂ for 30 minutes.
6. Add 6 ul/well 25×LPS. 500 ug/ml stock diluted in RPMI/20mM HEPES (75 ul stock+9.925 ml RPMI=3.75 ug/ml). 150 ng/ml final. Use RPMI/20 mM HEPES for neg. control wells.
7. Incubate at 37°/5% CO₂ for 2 hours.
8. Add 6 ul/well of 25×ATP. 0.5M stock diluted in RPMI/20 mM HEPES (2 ml stock+6 ml RPMI/20 mM HEPES=125 mM. 5 mM final. Use RPMI/20 mM HEPES for negative control wells.
9. Incubate at 37°/5% CO₂ for 2 hours.
10. Spin plates for 10 minutes at 1000 rpm.
11. Collect 70 ul plasma/well. Dilute 1:2 in dPBS/0.1% BSA for hIL-1b determination using MSD Single-spot Assay/Protocol. Ec50s are calculated from curves (% inhibition) using 1% DMSO/+LPS/−ATP controls as background signal, and 1% DMSO/+LPS/+ATP treated controls as 100% signal.
   Reagents:
   DMSO: Sigma, Cat.# D2650
   HEPES: Invitrogen, Cat.# 1530-080
   RPMI: Invitrogen, Cat.# 21870-076
   LPS: Calbiochem #437625, 055:B5.
   ATP: Amersham # 27-1006.
   IL-1b Assay: Meso Scale Discovery, hIL-1b single-spot assay, Cat # L411AGB-1, read on Sector 6000 reader.
   Mouse Whole Blood Assay Protocol:
1. Collect Balb/C mouse blood, by cardiac puncture, into 5 ml tubes (green top, vacutainer).
2. Put 200 ul pooled Balb/C mouse blood (diluted 1:1 with RPMI/20 mM HEPES)/well (96 well, flat bottomed plate Costar # 3599).
3. Compound Dilution: Done in 96 well, round bottom polypropylene plates.
   a. Take 10 mM stock (100% DMSO) and dilute 1:5 (10 ul+40 ul DMSO) in row A (one compound/column)=2 mM in 100% DMSO.
   b. Add 40 ul DMSO to wells in rows B-H. Make 1:5 serial dilutions of the 2 mM compounds (10 ul+40 ul DMSO) down to row F. Rows G and H are no drug treatment wells.
   c. Dilute the dilutions 1:4 in RPMI+20 mM HEPES (10 ul+30 ul medium)=25× compound in 25% DMSO in a new plate, making duplicate columns for each drug (5 drugs/plate).
   d. Transfer 8.5 ul/well of each dilution into blood plate wells. This is a 1:25 dilution that will give 1× compound in 1% DMSO final concentration Add 8.5 ul 25% DMSO to control wells.
4. Add 20 ul LPS (11.4 ug/ml stock =1 ug/ml final conc.) to all wells except (−) LPS wells (RPMI/20 mM HEPES). Incubate at 37° C., 5% CO₂ for 1.5 hrs.
5. Add 20 ul 12.4 mM ATP (7.5 mg/ml) so final conc. is 1 mM. Incubate for 2 hrs at 37° C., 5% CO₂.
6. Spin down Plate (10 minutes, 1000 RPM), remove plasma and dilute 1:3 in separate plate (dPBS/0.1% BSA). Measure IL-1b in samples using R&D, IL-1b kit/protocol. Ec50 values calculated from Prism curves using LPS/no ATP controls as background signal, and LPS/ATP treated controls as 100% signal.

Reagents:

DMSO: Sigma, Cat.# D2650,

HEPES: Invitrogen, Cat.# 1530-080

RPMI: Invitrogen, Cat.# 21870-076

LPS: Calbiochem # 437625, 055:B5.

ATP: Amersham # 27-1006.

R&D m IL-1b ELISA kit: MLB00

The in vivo potency of compounds in inhibiting P2X7 is discussed herein or described in the art may be determined by the procedure detailed below.

Collagen-Induced Arthritis Protocol

Mice were immunized with 100 μl of a 1:1 emulsion of bovine type II collagen (100 μg per mouse) in complete Freund's adjuvant (CFA) intradermally at the base of the tail. A boost with 1 mg Zymosan A was given i.p. on day 21. (This procedure has been adapted from Joosten L A et al). The arthritis was graded clinically according to the number of joints involved, presence of redness and swelling at one or more sites, deformity in the paws, and stiffness in the joints (ankylosis). Paw swelling was measured by calipers. Test compounds were administered PO, before onset or after onset of arthritis. The immunized mice were followed up for 2-3 weeks after onset of disease after which they were euthanized.

Animals:

Strain: DBA1/J, Age: ≧6 weeks, Size: 20-35 grams

Source: Jackson Labs, Bar Harbor, Me.

Specialized Instruments and Procedures:

Arthritic animals are maintained on Transgel (CRL). The arthritis was graded clinically on a score of 0-3 according to presence of redness and swelling at one or more sites (1), two or more sites (2), or deformity in the paws and stiffness in the joints (ankylosis) (3). Paw swelling was measured by calipers.

### REFERENCES

Joosten L A, Helsen M M and van den Berg W B (1994). Accelerated onset of collagen-induced arthritis by remote inflammation. Clin Exp Immunol: 97:204-11.

### EXPERIMENTALS

### Abbreviations

DMF N,N-Dimethylformamide
DMA N,N-Dimethylacetamide
SEM 2-(Trimethylsilyl)ethoxymethyl
PMB p-Methoxybenzyl
Cbz Benzyloxycarbonyl
TMS Trimethylsilyl
Boc tert-Butoxycarbonyl
RP Reverse Phase
HPLC High Performance Liquid Chromatography
Rₜ Retention time
TBDMS tert-Butyldimethylsilyl
THF Tetrahydrofuran
HOAc Acetic acid
i-PrOH 2-Propanol
t-BuOH tert-Butyl alcohol
t-BuOK Potassium tert-butoxide
Et₂O Diethyl ether
EtOAc Ethyl acetate
DME 1,2-Dimethoxyethane
Racemic-BINAP (±)-2,2′-Bis(diphenylphosphino)-1,1′-binaphthalene
(R)-BINAP(R)-(+)-2,2′-Bis(diphenylphosphino)-1,1′-binaphthalene
(S)-BINAP (S)-(−)-2,2′-Bis(diphenylphosphino)-1,1′-binaphthalene
DPPF 1,1′-Bis(diphenylphosphino)ferrocene
TFA Trifluoroacetic acid
DCC N,N′-Dicyclohexylcarbodiimide
DIC N,N′-Diisopropylcarbodiimide
EDC 1-(3-Dimethylaminopropyl)-3-ethylcarbodiimide
HBTU O-Benzotriazol-1-yl-N,N,N′,N′-tetramethyluronium hexafluorophosaphate
HATU O-(7-Azabenzotriazol-1-yl)-N,N,N′,N′-tetramethyluronium hexafluorophosaphate
TFFH Fluoro-N,N,N′,N′-tetramethylformamidinium hexafluorophosphate
HOBT 1-Hydroxybenzotriazole
HOAT 1-Hydroxy-7-azabenzotriazole
DIEA N,N-Diisopropylethylamine
XANTPHOS 9,9-Dimethyl-4,5-bis(diphenylphosphino)xanthene
KOAc Potassium acetate
DMSO Dimethyl sulfoxide
LDA Lithium diisopropylamide
PPh₃ Triphenylphosphine
Et₃N Triethylamine
PPTS Pyridinium p-toluenesulfonate
DMFDMA N,N-Dimethylformamide dimethyl acetal
TBAF tetra-n-Butylammonium fluoride
MP-carbonate Polymer bound tetraalkylammonium carbonate
Si-DCT Silica bound dichlorotriazine
CDI 1,1′-Carbonyldiimidazole
ThioCDI 1,1′-Thiocarbonyldiimidazole
Fmoc 9-Fluorenylmethoxycarbonyl
DCM Dichloromethane
MeOH Methanol
MeCN Acetonitrile
n-PrOH 1-Propanol
EtOH Ethanol
m-CPBA 3-Chloroperbenzoic acid
   Synthetic Details

Analytical data is included either in the illustrations of the general procedures or in the tables of examples. Unless otherwise stated, all ¹H or ¹³C NMR data were collected on a Varian Mercury Plus 400 MHz or a Bruker DRX 400 MHz instrument; chemical shifts are quoted in parts per million (ppm). High pressure liquid chromatography (HPLC) analytical data are either detailed within the experimental or referenced to the Table of HPLC conditions, using the lower case method letter.

Table of HPLC conditions

| | HPLC Conditions |
|---|---|
| | Unless indicated otherwise mobile phase A was 10 mM ammonium acetate, |
| Method | mobile phase B was HPLC grade acetonitrile. |
| a | 5-95% B over 3.7 min with a hold at 95% B for 1 min (1.3 mL/min flow |
| | rate). 4.6 × 50 mm Waters Atlantis dC18 column (5 μm particles). |
| | Detection methods are diode array (DAD) and evaporative light scattering |
| | (ELSD) detection as well as pos/neg electrospray ionization. |
| b | 5-60% B over 1.5 min then 60-95% B to 2.5 min with a hold at 95% B for |
| | 1.2 min (1.3 mL/min flow rate). 4.6 × 30 mm Vydac Genesis C8 column (4 μm |
| | particles). Detection methods are diode array (DAD) and evaporative |
| | light scattering (ELSD) detection as well as pos/neg electrospray ionization. |
| c | 5-60% B over 1.5 min then 60-95% B over 2.5 min with a hold at 95% B for |
| | 1.2 min (1.3 ml/min flow rate). 4.6 × 50 mm Zorbax XDB C8 column (5 μm |
| | particles). Detection methods are diode array (DAD) and evaporative light |
| | scattering (ELSD) detection as well as pos/neg electrospray ionization. |
| d | 30% to 95% B over 2.0 min; 95% B for 1.5 min at 1.0 ml/min; UV λ = 210-360 nm; |
| | Genesis C8, 4 μm, 30 × 4.6 mm column; ESI +ve/−ve. |
| e | 10% to 40% B over 4.0 min; 40% to 95% B over 2.0 min; 95% B for 1.0 min |
| | at 1.0 ml/min; UV λ = 210-360 nm; Genesis C8, 4 μm, 30 × 4.6 mm column; |
| | ESI +ve/−ve. |
| f | 5% to 95% B over 2.0 min; 95% B for 1.5 min at 1.4 ml/min; UV λ = 210-360 nm; |
| | Genesis C8, 4 μm, 30 × 4.6 mm column; ESI +ve/−ve. |
| g | 30% to 95% B over 2.0 min; 95% B for 3.5 min at 1.0 ml/min; UV λ = 190-400 nm; |
| | Genesis C8, 4 μm, 30 × 4.6 mm column; ESI +ve/−ve. |
| h | 5% to 35% B over 4.0 min; 35%-95% B over 2 min; 95% B for 1.0 min at |
| | 1.0 ml/min; UV λ = 190-400 nm; Genesis C8, 4 μm, 30 × 4.6 mm column; |
| | ESI +ve/−ve. |
| i | 30% to 95% B over 3.0 min; 95% B for 1.5 min at 0.8 ml/min; UV λ = 210-350 nm; |
| | Genesis C18, 3 μm, 33 × 4.6 mm column; ESI +ve/−ve. |
| j | Analytical LC-MS was performed on a Waters ZMD mass spectrometer and |
| | Alliance HPLC system running MassLynx 3.4 and Openlynx 3.4 software. |
| | The ZMD mass spectrometer was operated under positive APCI ionization |
| | conditions. The HPLC system comprised a Waters 2795 autosampler |
| | sampling from 96-well plates, a Waters 996 diode-array detector and Sedere |
| | Sedex-75 evaporative light scattering detector. The column used was a |
| | Phenomenex Luna Combi-HTS C8(2) 5 μm 100 Å (2.1 mm × 30 mm). A |
| | gradient of 10-100% acetonitrile (A) and 0.1% trifluoroacetic acid in water |
| | (B) was used, at a flow rate of 1.5 mL/min (0-0.1 min 10% A, 0.1-3.1 min |
| | 10-100% A, 3.1-3.9 min 100-10% A, 3.9-4.0 min 100-10% A). |
| k | 5% to 95% B over 3.0 min; 95%-100% B over 0.7 min; 95%-5% B over |
| | 0.1 min; 5% B for 0.2 min at 2.0 ml/min; Mobile phase A was 5 mM |
| | ammonium acetate; UV λ = 254 nm; Pecosphere C18, 3 μm, 80a, 33 × 4.6 mm |
| | column; ESI +ve/−ve. |
| l | 5% to 95% B over 3.0 min; 95%-100% B over 0.7 min; 95%-5% B over |
| | 0.1 min; 5% B for 0.2 min at 1.3 ml/min; Mobile phase A was 5 mM |
| | ammonium acetate; UV λ = 254 nm; Genesis C8, 4 μm, 30 × 4.6 mm |
| | column; ESI +ve/−ve. |
| m | CI method used isobutane as carrier/ionization gas. |
| | GC column used for all listed methods was Restek RTX-5MS |
| | (10 mL × .25 mm ID × .25 μm film thickness) |
| | 2 min wait prior to MS detection to avoid solvent peak detection. |
| | Initial 100° C., hold 1 min; 30° C./min to 340° C., hold 3 min; 12 min run. |

List of General Procedures
General Procedure A: Carbamimidate formation
General Procedure B: Cyanoguanidine formation from a carbamimidate
General Procedure C: Urea formation
General procedure D: Amide formation
General Procedure E: Sulfonamide formation
General Procedure F: Isothiocyanate formation
General Procedure G: Cyanoguanidine and thiourea formation from an isothiocyanate
General Procedure H: Heteroarylamine formation from a haloheteroarene
General Procedure I: Quinoline-N-oxide formation
General Procedure J: Reductive amination
General procedure K: Suzuki reaction
General Procedure L: Cbz protection of an amine
General Procedure M: Cbz deprotection
General Procedure N: Thiourea formation
General Procedure 0: Fmoc protection of an amine
General Procedure P: Fmoc deprotection
General Procedure Q: Arylamide formation by palladium-catalyzed carbonylation

The general synthetic schemes that were utilized to construct the majority of compounds disclosed in this application are described below in (Schemes 1-28).

The general procedure letter codes constitute a synthetic route to the final product. 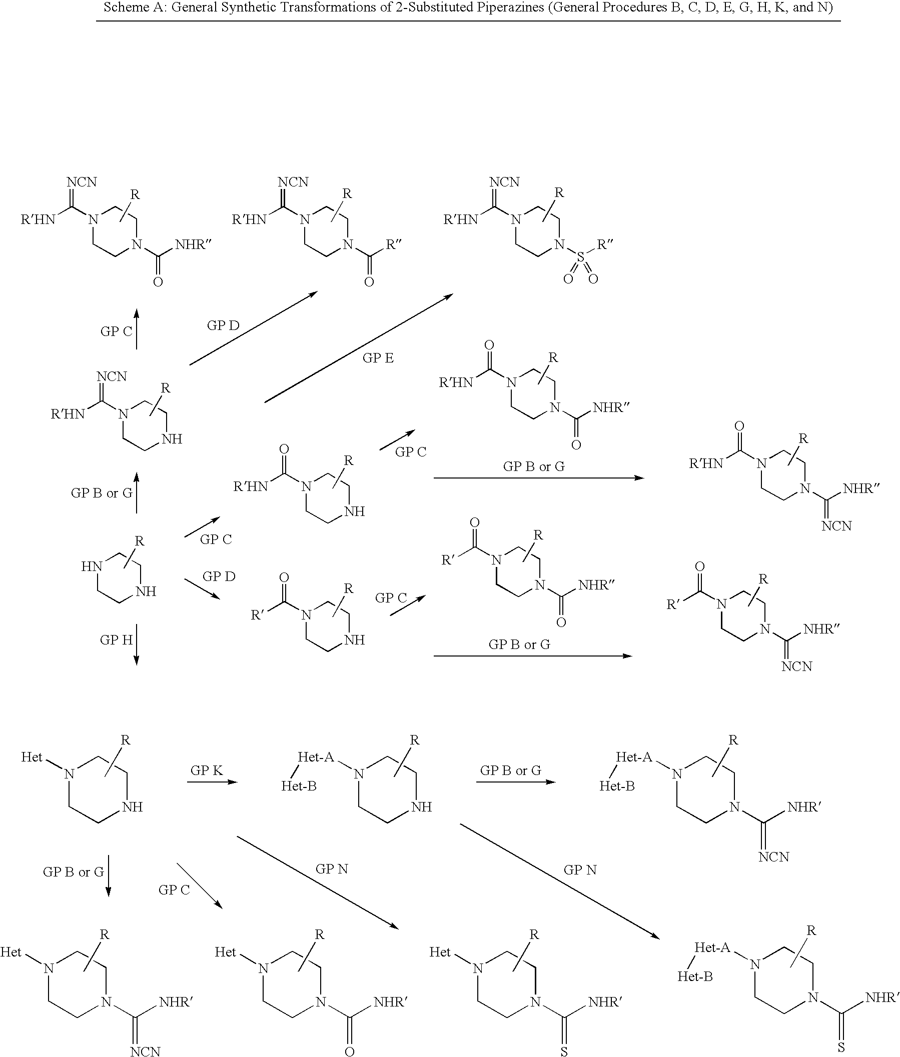
General Procedure
General Procedure A: Carbamimidate Formation

A mixture of the amine (1-11 equivalents, preferably 1 equivalent) and diphenylcyanocarboimidate (1 equivalent) is heated in acetonitrile at about 80° C. for 4-18 hours (preferably 18 hours) under inert atmosphere. The mixture is allowed to cool to ambient temperature before the reaction volume is approximately doubled with ether. The resulting precipitate is filtered and washed with ether to afford the product.

Illustration of General Procedure A

### Preparation 1: Phenyl N′-cyano-N-o-tolylcarbamimidate

A mixture of o-toluidine (22.8 ml, 212.4 mmol) and diphenylcyanocarboimidate (50.6 g, 212.4 mmol) in acetonitrile (100 ml) was heated at about 80° C. for 18 hours under nitrogen atmosphere. The mixture was allowed to cool to ambient temperature, then ether (100 ml) was added. The resulting precipitate was filtered and washed with ether (100 ml) to give (Z)-phenyl N′-cyano-N-o-tolylcarbamimidate as a white solid (5.5 g, 21.8 mmol); ¹HNMR (DMSO-d₆, 400 MHz) δ 10.49 (s, 1H), 7.43 (m, 2H), 7.37 (m, 1H), 7.31 (m, 2H), 7.25 (m, 4H), 2.31 (s, 3H); RP-HPLC (Method i) Rₜ 2.57 min; MS m/z: (M−H)⁻ 250.

General Procedure B: Cyanoguanidine Formation from a Carbamiimidate

To a mixture of the amine (1 equivalent) and an organic base (preferably triethylamine; 1-2 equivalents, preferably 1 equivalent) in an organic solvent (preferably acetonitrile) is added the carbamimidate (1 equivalent). The reaction is heated at about 80° C. for 5-18 hours (preferably 5 hours) under inert atmosphere. The mixture is allowed to cool to ambient temperature and is concentrated in vacuo. The product is purified by chromatography.

Illustration of General Procedure B

### Preparation 2: N′-Cyano-3-phenyl-N-o-tolylpiperazine-1-carboximidamide

To a mixture of 2-phenylpiperazine (15.0 g, 92.46 mmol) and triethylamine (13.0 mL, 92.46 mmol) in acetonitrile (100 ml) was added phenyl-N′-cyano-N-o-tolylcarbamimidate (23.2 g, 92.46 mmol). The reaction was heated at about 80° C. for about 5 hours under a nitrogen atmosphere. The mixture was allowed to cool to ambient temperature and was concentrated in vacuo. The residue was purified by flash chromatography on silica gel using 1% methanol in dichloromethane as the mobile phase to give N′-cyano-3-phenyl-N-o-tolylpiperazine-1-carboximidamide as a white foam (13.8 g, 43.2 mmol); ¹HNMR (DMSO-d₆, 400 MHz) δ 8.89 (s, 1H), 7.38-7.05 (m, 9H), 3.96 (t, 2H), 3.65 (m, 1H), 2.99 (m, 2H), 2.75 (m, 2H), 2.20 (s, 3H); RP-HPLC (Method i) Rₜ 1.85 min; MS m/z: (M+H)⁺ 250.

General Procedure C: Urea Formation

At 0° C. or ambient temperature (preferably 0° C.), a solution of the isocyanate (1.0-1.5 equivalents, preferably 1.0 equivalent) in an organic solvent (for example tetrahydrofuran, dichloromethane or acetonitrile, preferably tetrahydrofuran) is added dropwise to an organic solution of the amine (1 equivalent) in an organic solvent (for example tetrahydrofuran, dichloromethane or acetonitrile, preferably tetrahydrofuran). The mixture is allowed to stir at ambient temperature for 2-18 hours (preferably 2 hours) before quenching the reaction by addition of water. The product is purified by chromatography or by filtration of the precipitate after dilution with ether (preferably chromatography).

Illustration of General Procedure C

### Example 1

### N-(4-Chlorophenyl)-4-(N′-cyano-N-o-tolylcarbamimidoyl)-2-phenylpiperazine-1-carboxamide

At 0° C., a solution of 4-chlorophenylisocyanate (1.63 g, 10.64 mmol) in tetrahydrofuran (10 mL) was added dropwise to a solution of the N′-cyano-3-phenyl-N-o-tolylpiperazine-1-carboximidamide (3.40 g, 10.64 mmol) in tetrahydrofuran (20 mL). The mixture was allowed to stir at ambient temperature for 2 hours before quenching the reaction by the addition water (0.5 mL). The reaction was diluted with ether (30 mL). The resulting precipitate was collected by filtration, washed with ether (30 mL) and dried in vacuo to give N-(4-chlorophenyl)-4-(N′-cyano-N-o-tolylcarbamimidoyl)-2-phenylpiperazine-1-carboxamide as a white solid (4.4 g, 9.3 mmol); ¹HNMR (DMSO-d₆, 400 MHz) δ 8.85 (s, 1H), 8.75 (s, 1H), 7.49 (m, 2H), 7.32 (m, 7H), 7.18 (m, 1H), 7.08 (m, 2H), 6.72 (m, 1H), 5.50 (m, 1H), 4.45 (m, 1H), 4.02 (m, 1H), 3.75 (m, 2H), 3.38 (m, 2H), 2.05 (s, 3H); RP-HPLC (Method i) Rₜ 2.78 min; MS m/z: (M+H)⁺ 473.

General Procedure D: Amide Formation

At 0° C. or ambient temperature (preferably 0° C.), a solution of the acid chloride or carboxylic acid that is activated by an equimolar amount of coupling reagent (for example EDC, HATU, CDI or 2-chloro-4,6-dimethoxy-1,3,5-triazine with N-methylmorpholine, preferably EDC) (1.0-1.5 equivalents, preferably 1.0 equivalent) in an organic solvent (for example tetrahydrofuran, dichloromethane or acetonitrile, preferably dichloromethane) is added dropwise to an organic solution of the amine (1 equivalent) and an organic base (for example triethylamine, diisopropylethylamine or pyridine, preferably triethylamine; 1.0-3.0 equivalents, preferably 2.0 equivalents) in an organic solvent (for example tetrahydrofuran, dichloromethane or acetonitrile, preferably dichloromethane). The mixture is allowed to stir at ambient temperature for 2-18 hours (preferably 2 hours) before quenching the reaction by the addition of water. The solvent is removed in vacuo before the product is purified by reverse-phase HPLC.

Illustration of General Procedure D

### Example 2

### 4-(2-(4-Chlorophenyl)acetyl)-N′-cyano-3-phenyl-N-o-tolylpiperazine-1-carboximidamide

At 0° C., a solution of 4-chlorophenylacetylchloride (49.0 mg, 0.258 mmol) in dichloromethane (1 mL) was added dropwise to a solution of N′-cyano-3-phenyl-N-o-tolylpiperazine-1-carboximidamide (75 mg, 0.235 mmol) and pyridine (0.060 mL, 0.704 mmol) in dichloromethane (1 mL). The mixture was allowed to stir at ambient temperature for 18 hours before quenching the reaction by the addition of water (0.1 mL). The solvent was removed in vacuo before the product was purified by reverse-phase HPLC on a Hyperprep HS C18 column, 8 4m, 250×21.2 mm; 20% acetonitrile-50 mM ammonium acetate over 1 min, 20-60% acetonitrile-50 mM ammonium acetate for 34 min, 60-100% acetonitrile for 1 min, 100% acetonitrile for 5 min, 25 mL/min. The product was isolated by lyophilization of the desired fractions to give 4-(2-(4-chlorophenyl)acetyl)-N′-cyano-3-phenyl-N-o-tolylpiperazine-1-carboximidamide (17 mg, 0.036 mmol); ¹HNMR (DMSO-d₆, 400 MHz) δ 8.85 (bs, 1H), 7.37 (m, 4H), 7.30 (m, 4H), 7.16 (m, 1H), 7.06 (m, 3H), 6.72 (m, 1H), 5.50 (m, 1H), 4.42 (m, 1H), 3.95 (m, 3H), 3.69 (m, 2H), 3.38 (m, 2H), 2.05 (s, 3H); RP-HPLC (Method i) Rₜ 2.92 min; MS m/z: (M+H)⁺ 472.

General Procedure E: Sulfonamide Formation

At 0° C. or ambient temperature (preferably 0° C.), a solution of the sulfonyl chloride (1.0-1.5 equivalents, preferably 1.0 equivalent) in an organic solvent (for example tetrahydrofuran, dichloromethane or acetonitrile, preferably dichloromethane) is added dropwise to an organic solution of the amine (1 equivalent) and an organic base (for example triethylamine, diisopropylethylamine or pyridine, preferably triethylamine; 1.0-3.0 equivalents, preferably 2.0 equivalents) in an organic solvent (for example tetrahydrofuran, dichloromethane or acetonitrile, preferably dichloromethane). The mixture is allowed to stir at ambient temperature for 2-18 hours (preferably 2 hours) before quenching the reaction by the addition of water. The solvent is removed in vacuo before the product is purified by reverse-phase HPLC.

Illustration of General Procedure E

### Example 3

### 4-(4-Chlorophenylsulfonyl)-N′-cyano-3-phenyl-N-o-tolylpiperazine-1-carboximidamide

At 0° C., a solution of 4-chlorophenylsulfonylchloride (33.0 mg, 0.157 mmol) in dichloromethane (0.5 mL) was added dropwise to a solution of N′-cyano-3-phenyl-N-o-tolylpiperazine-1-carboximidamide (50 mg, 0.157 mmol) and pyridine (0.025 mL, 0.313 mmol) in dichloromethane (0.5 mL). The mixture was allowed to stir at ambient temperature for 18 hours before quenching the reaction by the addition of water (0.1 mL). The solvent is removed in vacuo before the product was purified by reverse-phase HPLC on a Hyperprep HS C18 column, 8 μm, 250×21.2 mm; 20% acetonitrile-50 mM ammonium acetate over 1 min, 20-60% acetonitrile-50 mM ammonium acetate for 34 min, 60-100% acetonitrile for 1 min, 100% acetonitrile for 5 min, 25 mL/min. The product was isolated by lyophilization of the desired fractions to give 4-(4-chlorophenylsulfonyl)-N′-cyano-3-phenyl-N-o-tolylpiperazine-1-carboximidamide (5.0 mg, 0.010 mmol); ¹HNMR (CDCl₃, 400 MHz) δ 7.62 (m, 2H), 7.42 (m, 2H), 7.35 (m, 3H), 7.21 (m, 2H), 7.15 (m, 1H), 7.07 (m, 1H), 6.90 (m, 1H), 6.46 (s, 1H), 6.37 (m, 1H), 5.11 (s, 1H), 4.20 (d, 1H), 3.54 (m, 1H), 3.38 (m, 1H), 3.27 (dd, 1H), 3.16 (m, 1H), 2.91 (m, 1H), 2.07 (s, 3H); RP-HPLC (Method i) Rₜ 3.12 min; MS m/z: (M+H)⁺ 494.

General Procedure F: Isothiocyanate Formation

At 0° C. or ambient temperature (preferably 0° C.), 1,1′-thiocarbonyldiimidazole (1.0-1.2 equivalents, preferably 1.0 equivalent) is added to a solution of the amine (1.0 equivalent) in an organic solvent (for example tetrahydrofuran, dichloromethane or acetonitrile, preferably dichloromethane). The mixture is allowed to stir at ambient temperature for 2-18 hours (preferably 2 hours) before the solvent is removed in vacuo. The product is purified by chromatography.

Illustration of General Procedure F

### Preparation 3: 5-Isothiocyanatoquinoline

At 0° C., 1,1′-thiocarbonyldiimidazole (12.94 g, 72.62 mmol) was added to a solution of 5-aminoquinoline (10.47 g, 72.62 mmol) in dichloromethane (40 mL). The mixture is allowed to stir at ambient temperature for 2 hours before the solvent is removed in vacuo. The residue was purified by flash chromatography on silica gel using dichloromethane as the mobile phase to give 5-isothiocyanatoquinoline as a pale yellow solid (11.40 g, 61.21 mmol); ¹HNMR (CDCl₃, 400 MHz) δ 9.00 (m, 1H), 8.49 (m, 1H), 8.09 (m, 1H), 7.69 (m, 1H), 7.55 (m, 2H); RP-HPLC (Method d) Rₜ 2.26 min; MS m/z: (M+H)⁺ 187.

General Procedure G: Cyanoguanidine and Thiourea Formation from an Isothiocyanate

At ambient temperature, the isothiocyanate (1.0-1.2 equivalents, preferably 1.0 equivalent) is added to a suspension of the sodium hydrogen cyanamide (1.0-1.2 equivalents, preferably 1.0 equivalent) in an organic solvent (for example tetrahydrofuran, N,N-dimethylformamide or ethanol, preferably N,N-dimethylformamide). The mixture is allowed to stir at ambient temperature for 0.2-4.0 hours (preferably 0.5 hours) before the addition of zinc dichloride (0-1 equivalents, preferably 1 equivalent or 0 equivalents). At ambient temperature, N-(3-dimethylaminopropyl)-N′-ethylcarbodiimide hydrochloride (1.0-1.2 equivalents, preferably 1.1 equivalents) is premixed with the amine (1.0 equivalent) in an organic solvent (for example tetrahydrofuran, N,N-dimethylformamide or ethanol, preferably N,N-dimethylformamide) before it is added to the reaction dropwise. The reaction is allowed to stir at ambient temperature for 1-18 hours (preferably 6 hours). For reactions containing zinc dichloride, the reaction is partitioned with organic solvent (for example dichloromethane or ethyl acetate, preferably dichloromethane) and brine. The organic layer is dried with sodium sulfate or magnesium sulfate before it is filtered. The solvent is removed in vacuo before the product(s) are purified by reverse-phase HPLC.

Illustration of General Procedure G

### Example 4

### N-(4-Chlorophenyl)-4-(N′-cyano-N-(2-methylquinolin-5-yl)carbamimidoyl)-3-isopropylpiperazine-1-carboxamide and

### Example 5

### 3-isopropyl-4-(2-methylquinolin-5-ylthiocarbamoyl)piperazine-1-carboxylic acid(4-chlorophenyl)amide

At ambient temperature, 5-isothiocyanato-2-methylquinoline (611 mg, 3.05 mmol) was added to a suspension of the sodium hydrogen cyanamide (195 mg, 3.05 mmol) in N,N-dimethylformamide (6 mL). At ambient temperature, N-(3-dimethylaminopropyl)-N′-ethylcarbodiimide hydrochloride (585 mg, 3.05 mmol) was premixed with N-(4-chlorophenyl)-3-isopropylpiperazine-1-carboxamide (860 mg, 3.05 mmol) in N,N-dimethylformamide (6 mL) before it was added to the reaction dropwise 1 hour later. The reaction was allowed to stir at ambient temperature for 18 hours before the products were purified by reverse-phase HPLC on a Hyperprep HS C18 column, 8 μm, 250×21.2 mm; 20% acetonitrile-50 mM ammonium acetate over 1 min, 20-60% acetonitrile-50 mM ammonium acetate for 34 min, 60-100% acetonitrile for 1 min, 100% acetonitrile for 5 min, 25 mL/min. The products were isolated by lyophilization of the desired fractions to give N-(4-chlorophenyl)-4-(N′-cyano-N-(2-methylquinolin-5-yl)carbamimidoyl)-3-isopropylpiperazine-1-carboxamide (220 mg, 0.45 mmol); ¹HNMR (DMSO-d₆, 400 MHz) δ 8.72 (bs, 1H), 8.30 (m, 1H), 7.74 (m, 1H), 7.66 (m, 1H), 7.49 (m, 3H), 7.29 (m, 2H), 7.22 (m, 1H), 4.30 (m, 1H), 4.12 (m, 2H), 3.98 (m, 1H), 3.23 (m, 1H), 3.05 (m, 2H), 2.67 (s, 3H), 2.05 (m, 1H), 0.95 (m, 6H); RP-HPLC (Method d) Rₜ 1.76 min; MS m/z: (M+H)⁺ 490 and 3-isopropyl-4-(2-methylquinolin-5-ylthiocarbamoyl)piperazine-1-carboxylic acid(4-chlorophenyl)amide (23 mg, 0.05 mmol); ¹HNMR (DMSO-d₆, 400 MHz) δ 8.74 (bs, 1H), 8.06 (m, 1H), 7.82 (m, 1H), 7.68 (m, 1H), 7.52 (m, 2H), 7.38 (m, 1H), 7.30 (m, 3H), 4.34 (m, 2H), 4.17 (m, 1H), 3.28 (m, 1H), 3.09 (m, 3H), 2.65 (s, 3H), 2.14 (m, 1H), 1.01 (m, 6H); RP-HPLC (Method d) Rₜ 1.93 min; MS m/z: (M+H)⁺ 482.

General Procedure H: Heteroarylamine Formation from a Haloheteroarene

The haloheteroarene (1.0-1.5 equivalents, preferably 1.0 equivalent) is added to the amine (1 equivalent) in an organic solvent (for example tetrahydrofuran, dichloromethane, N,N-dimethylformamide, acetonitrile or 1-propanol, preferably 1-propanol). The mixture is stirred at 25-170° C. (preferably at 120° C.). An oil bath or a microwave oven can be used for heating if necessary (preferably microwave oven) for 0.3-18 hours (preferably 0.3 or 6 hours). The product is purified by chromatography or by filtration of the precipitate (preferably filtration of the precipitate).

Illustration of General Procedure H

### Preparation 4: 2-(3-Isopropylpiperazin-1-yl)benzo[d]oxazole

At ambient temperature, 2-chlorobenzoxazole (1.81 mL, 15.60 mmol) was added to a solution of 2-isopropylpiperazine (2.00 g, 15.60 mmol) in dichloromethane (20 mL). The reaction was stirred at ambient temperature for 0.5 hour before it was diluted with brine. The organic layer was separated and was dried with sodium sulfate. The solvent is removed in vacuo before the product was purified by silica gel chromatography using 3% methanol in dichloromethane to give 2-(3-isopropylpiperazin-1-yl)benzo[d]oxazole (1.69 g, 6.89 mmol); ¹HNMR (DMSO-d₆, 400 MHz) δ 7.38 (m, 1H), 7.27 (m, 1H), 7.14 (m, 1H), 7.00 (m, 1H), 3.97 (m, 2H), 3.00 (m, 2H), 2.79 (t, 1H), 2.68 (m, 1H), 2.36 (m, 1H), 1.60 (m, 1H), 0.95 (m, 6H); RP-HPLC (Method d) Rₜ 1.00 min; MS m/z: (M+H)⁺ 246.

General Procedure I: Quinoline-N-Oxide Formation

At ambient temperature, m-CPBA (1.0-1.3 equivalents, preferably 1.1 equivalents) is added to a solution of the quinoline (1.0 equivalent) in dichloromethane. The mixture is allowed to stir at ambient temperature for 6-18 hours (preferably 18 hours) before the reaction is partitioned with saturated sodium bicarbonate solution. The organic layer is separated and dried with sodium sulfate or magnesium sulfate before the solvent is removed in vacuo. The product is purified by reverse-phase HPLC.

Illustration of General Procedure I

### Example 6

### 5-(3-Ethyl-2-ethynyl-3-(1-(3-(3-fluorophenyl)ureido)-3-methylbutan-2-yl)guanidino)quinoline-1-oxide

At ambient temperature, m-CPBA (341 mg, 1.38 mmol) was added to a solution of the 4-(N′-cyano-N-(quinolin-5-yl)carbam imidoyl)-N-(3-fluorophenyl)-3-isopropylpiperazine-1-carboxamide (489 mg, 1.06 mmol) in dichloromethane (20 mL). The mixture was allowed to stir at ambient temperature for 18 hours before the reaction was partitioned with saturated sodium bicarbonate solution. The organic layer was separated and dried with sodium sulfate before the solvent was removed in vacuo. The product was purified by reverse-phase HPLC on a Hyperprep HS C18 column, 8 μm, 250×21.2 mm; 20% acetonitrile-50 mM ammonium acetate over 1 min, 20-50% acetonitrile-50 mM ammonium acetate for 34 min, 50-100% acetonitrile for 1 min, 100% acetonitrile for 5 min, 25-mL/min. The product was isolated by lyophilization of the desired fractions to give 5-(3-ethyl-2-ethynyl-3-(1-(3-(3-fluorophenyl)ureido)-3-methylbutan-2-yl)guanidino)quinoline-1-oxide (220 mg, 0.46 mmol); ¹HNMR (DMSO-d₆, 400 MHz) δ 8.80 (s, 1H), 8.61 (m, 1H), 8.33 (m, 1H), 7.96 (m, 1H), 7.75 (m, 1H), 7.51 (m, 1H), 7.43 (m, 2H), 7.26 (m, 2H), 6.75 (m, 1H), 4.34 (m, 1H), 4.15 (m, 2H), 4.04 (m, 1H), 3.20 (m, 1H), 3.08 (m, 2H), 2.07 (m, 1H), 0.96 (m, 6H); RP-HPLC (Method d) Rₜ 1.38 min; MS m/z: (M+H)⁺ 476.

General Procedure J: Reductive Amination

At ambient temperature, an aldehyde (1.0 equivalent) is added to a solution of a secondary amine (1.0 equivalent) in N,N-dimethylformamide. After the complete addition of the aldehyde, sodium triacetoxyborohydride (1.0-1.2 equivalents, preferably 1.1 equivalents) followed by a catalytic amount of glacial acetic acid is added to the reaction. The mixture is allowed to stir at ambient temperature for 6-18 hours (preferably 18 hours) before the reaction is partitioned with a saturated solution of sodium bicarbonate and dichloromethane. The organic layer is separated and dried with sodium sulfate or magnesium sulfate before the solvent is removed in vacuo. The product is purified by chromatography.

Illustration of General Procedure J

### Preparation 5: 2-(Cyclopropylmethyl)-1,2,3,4-tetrahydroisoquinolin-5-amine

At ambient temperature, cyclopropanecarbaldehyde (0.504 ml, 6.75 mmol) was added to a solution of the 1,2,3,4-tetrahydro-5-aminoisoquinoline (1.00 g, 6.75 mmol) in N,N-dimethylformamide (5 mL). After the complete addition of the aldehyde, sodium triacetoxyborohydride (1.50 g, 7.08 mmol) followed by a catalytic amount of glacial acetic acid (0.02 mL, 0.40 mmol) was added to the reaction. The mixture was allowed to stir at ambient temperature 18 hours before partitioning with a saturated solution of sodium bicarbonate and dichloromethane. The organic layer was separated and dried with sodium sulfate before the solvent was removed in vacuo. The product was purified by silica gel chromatography using 24% methanol in dichloromethane to give 2-(cyclopropylmethyl)-1,2,3,4-tetrahydroisoquinolin-5-amine (1.05 g, 5.19 mmol); ¹HNMR (DMSO-d₆, 400 MHz) δ 7.95 (bs, 2H), 6.80 (m, 1H), 6.43 (m, 1H), 6.25 (m, 1H), 3.49 (s, 2H), 2.70 (t, 2H), 2.45 (t, 2H), 2.30 (d, 2H), 0.90 (m, 1H), 0.49 (m, 2H), 0.12 (m, 2H); RP-HPLC (Method d) Rₜ 1.76 min; MS m/z: (M+H)⁺ 203.

General Procedure K: Suzuki Reaction

A mixture of 1 equivalent of aryl bromide or heteroaryl bromide or aryl chloride or heteroaryl chloride (preferably aryl bromide or heteroaryl bromide), aryl- or heteroaryl boronic acic or -boronic acid ester (preferably aryl- or heteroaryl boronic acid ester) (1.0-1.8 equivalents, preferably 1.5 equivalents), 2.5-3.5 equivalents (preferably 3.0 equivalents) of a base (cesium carbonate, potassium carbonate or sodium carbonate, preferably potassium carbonate) and [1,1-bis(diphenylphosphino)(ferrocene)]dichloropalladium(II), complex with dichloromethane (1:1) (0.04-0.1 equivalents, preferably 0.05 equivalents) is heated in the microwave in a mixture of water and organic solvent (DME, THF or dioxane, preferably DME) (water to organic solvent ratio ranges from 1 to 0.8 to 1 to 2, preferably 1 to 1) at 120-180° C., preferably 150° C. for 1040 minutes, preferably 25 minutes. The solution is concentrated under reduced pressure, the residue is suspended in an organic solvent (DCM, EtOAc or THF, preferably DCM) and the insoluble residue is filtered off. The filtrate is concentrated under reduced pressure and the crude product is purified by Chromatography or RP-HPLC or used for the next step without further purification.

Illustration of General Procedure K

### Preparation 6: 6-[6-(3-Isopropyl-piperazin-1-yl)-pyridazin-3-yl]-quinoline

A mixture of 3-chloro-6-(3-isopropyl-piperazin-1-yl)-pyridazine (0.057 g, 0.2 mmol), quinoline-6-boronic acid (0.051 g, 0.3 mmol), potassium carbonate (0.083 g, 0.6 mmol) and [1,1-bis(diphenylphosphino)(ferrocene)dichloropalladium(II), complex with dichloromethane (1:1) (0.008 g, 0.01 mmol) was heated in the microwave in a mixture of water (1 mL) and DME (1 mL) at 150° C. for 25 minutes. The solution was concentrated under reduced pressure, the residue was digested with DCM and the insoluble residue was filtered off. The filtrate was concentrated under reduced pressure and the crude product was used for the next step without further purification.

m/z: (M+H)⁺ 334.

General Procedure L: Cbz Protection of an Amine

At 0° C. or ambient temperature (preferably 0° C.), a solution of N-(benzyloxycarbonyloxy)succinimide or benzylchloroformate (1.0-2.0 equivalents, preferably 1.0 equivalent) in an organic solvent (for example tetrahydrofuran, dichloromethane or acetonitrile, preferably dichloromethane) is added dropwise to an organic solution of the amine (1 equivalent) and an organic base when benzylchloroformate is used (for example triethylamine, diisopropylethylamine or pyridine, preferably triethylamine; 1.0-3.0 equivalents, preferably 2.0 equivalents) in an organic solvent (for example tetrahydrofuran, dichloromethane or acetonitrile, preferably dichloromethane). The mixture is allowed to stir at ambient temperature for 2-18 hours (preferably 8 hours) before the reaction is partitioned with organic solvent (for example dichloromethane or ethyl acetate, preferably dichloromethane) and a saturated solution of sodium bicarbonate. The organic layer is dried with sodium sulfate or magnesium sulfate before it is filtered. The solvent is removed in vacuo. The product is used in subsequent reactions or is purified by chromatography (preferably chromatography).

Illustration of General Procedure L

### Preparation 7: Benzyl 5-amino-3,4-dihydroisoquinoline-2(1H)-carboxylate

At 0° C., a solution of N-(benzyloxycarbonyloxy)succinimide (21.27 g, 85.35 mmol) in dichloromethane (50 mL) was added dropwise to a solution of the 1,2,3,4-tetrahydro-5-aminoisoquinoline (12.65 g, 85.35 mmol) in dichloromethane (50 mL). The mixture was allowed to stir at ambient temperature for 8 hours before the reaction was partitioned with dichloromethane and a saturated solution of sodium bicarbonate. The organic layer was dried with sodium sulfate before it was filtered. The solvent was removed in vacuo to give benzyl 5-amino-3,4-dihydroisoquinoline-2(1H)-carboxylate as a tan solid (20.92 g, 74.10 mmol); ¹HNMR (DMSO-d₆, 400 MHz) δ 7.35 (m, 5H), 6.84 (m, 1H), 6.46 (m, 1H), 6.33 (m, 1H), 5.09 (s, 2H), 4.45 (bs, 2H), 3.63 (bs, 2H), 2.45 (m, 2H); RP-HPLC (Method d) Rₜ 1.95 min; MS m/z: (M+H)⁺ 283.

General Procedure M: Cbz Deprotection

The N-carbobenzyloxyamine (1 equivalent) and 10% palladium on carbon (0.1-0.3 equivalents) in an organic solvent (for example methanol, ethanol or ethyl acetate, preferably methanol) is shaken or vigorously stirred under hydrogen (1 atm-60 psi, preferably 60 psi) for 8-24 hours (preferably 12 hours) before the reaction is filtered through celite. The solvent is removed in vacuo. The product is used in subsequent reactions or is purified by chromatography (preferably chromatography).

Illustration of General Procedure M

### Example 7

### 4-(Benzo[d]oxazol-2-yl)-N′-cyano-2-isopropyl-N-(1,2,3,4-tetrahydroisoquinolin-5-yl)piperazine-1-carboximidamide

Benzyl 5-(4-(benzo[d]oxazol-2-yl)-N′-cyano-2-isopropylpiperazine-1-carboximidamido)-3,4-dihydroisoquinoline-2(1H)-carboxylate (0.70 g, 1.21 mmol) and 10% palladium on carbon (0.30 g, 0.28 mmol) in methanol (32 ml) were vigorously stirred under hydrogen (1 atm) for 18 hours before the reaction was filtered through celite. The solvent was removed in vacuo. The product was purified by reverse-phase HPLC on a Hyperprep HS C18 column, 8 gm, 250×21.2 mm; 20% acetonitrile-50 mM ammonium acetate over 1 min, 20-60% acetonitrile-50 mM ammonium acetate for 34 min, 60-100% acetonitrile for 1 min, 100% acetonitrile for 5 min, 25 mL/min. The product was isolated by lyophilization of the desired fractions to give 4-(benzo[d]oxazol-2-yl)-N′-cyano-2-isopropyl-N-(1,2,3,4-tetrahydroisoquinolin-5-yl)piperazine-1-carboximidamide (0.52 g, 1.20 mmol); ¹HNMR (DMSO-d₆, 400 MHz) δ 7.41 (m, 1H), 7.29 (m, 1H), 7.16 (m, 1H), 7.10 (m, 1H), 7.03 (m, 1H), 6.91 (m, 2H), 4.24 (m, 1H), 4.10 (m, 3H), 3.86 (s, 2H), 3.27 (m, 3H), 2.97 (m, 1H), 2.91 (m, 1H), 2.63 (m, 1H), 2.53 (m, 1H), 2.15 (m, 1H), 1.02 (m, 3H), 0.89 (m, 3H); RP-HPLC (Method d) Rₜ 1.47 min; MS m/z: (M+H)⁺ 444.

General Procedure N: Thiourea Formation

At ambient temperature, the isothiocyanate (1.0 equivalent) is added to the solution of the substituted piperazine (1.0-1.5 equivalents, preferably 1.1 equivalents) in an organic solvent (THF, DMF or ethanol, preferably DMF). The mixture is allowed to stir at ambient temperature for 0.5-6 hours, preferably 1 hour. The solvent is removed under reduced pressure and the residue purified by chromatography.

Illustration of General Procedure N

### Example 8

### 2-Isopropyl-4-[6-(1-methyl-1H-pyrazol-4-yl)-pyridazin-3-yl]-piperazine-1-carbothioic acid quinolin-5-ylamide

At ambient temperature, 5-isothiocyanato quinoline (0.235 g, 1.26 mmol) was added to the solution of crude 3-(3-isopropyl-piperazin-1-yl)-6-(1-methyl-1H-pyrazol-4-yl)-pyridazine (0.329 g, 1.15 mmol) in DMF (10 mL) and the mixture was allowed to stir for 1 hour. The solvent was removed under reduced pressure and the crude product was purified by preparative RP-HPLC (20% to 50% acetonitrile/0.05M aqueous ammonium acetate, buffered to pH 4.5, over 30 min at 21 mL/min; λ=254 nm; Microsorb C18, 100 Å, 5 μm, 250×46 mm column) to yield 2-isopropyl-4-[6-(1-methyl-1H-pyrazol-4-yl)-pyridazin-3-yl]-piperazine-1-carbothioic acid quinolin-5-ylamide (0.012 g, 0.025 mmol) as an off-white solid.

Retention time—1.31 min., RP-HPLC (30% to 95% B over 2.0 min; 95% B for 1.5 min at 1.0 ml/min; UV λ=210-360 nm; Genesis C8, 4 μm, 30×4.6 mm column; ESI +ve/−ve.)

m/z: (M−H)⁻ 471.

General Procedure 0: Fmoc Protection of an Amine

The Fmoc (1.0-1.5 equivalents, preferably 1.2 equivalents) is added to the amine (1 equivalents) in an organic solvent (for example tetrahydrofuran, dichloromethane, N,N-dimethylformamide, acetonitrile, preferably dichloromethane). The reaction is allowed to stir at 0-25° C. (preferably 25° C.) for 1-16h (preferably 3 hours). The product is purified by silica gel chromatography.

Illustration of General Procedure O

### Preparation 8: 5-Amino-3,4-dihydro-1H-isoquinoline-2-carboxylic acid 9H-fluoren-9-ylmethyl ester

At ambient temperature a solution of N-(9-fluorenylmethoxycarbonyloxy)succinimide (9.2 g, 27.3 mmol in 100 mL of dichloromethane) was slowly added to 1,2,3,4-tetrahydro-5-aminoisoquinoline (3.7 g, 24.8 mmol) in 100 mL of dichloromethane via addition funnel over 1 hour. The reaction was allowed to stir at ambient temperature for a further 2 hours after which the reaction was concentrated. The crude material was purified by silica gel chromatography employing a 60/40 mixture of ethyl acetate heptane as eluent to give 5-amino-3,4-dihydro-1H-isoquinoline-2-carboxylic acid 9H-fluoren-9-ylmethyl ester 5.7 g (15.4 mmol). ¹HNMR (DMSO-d₆, 400 MHz) δ 7.75 (m, 2H), 7.6 (s, 2H), 7.4-7.2 (m, 4H), 7.0 (s, 1H), 6.55 (m, 2H), 4.59 (s, 2H), 4.45 (m, 2H), 4.25 (t, 1H), 3.70 (d, 4H), 2.45 (s, 2H). RP-HPLC (Method d) Rₜ 1.00 min; MS m/z: (M+H)⁺ 246.

General Procedure P: Fmoc Deprotection

The Fmoc-protected amine is dissolved in 20% piperidine in DMF (3 to 200 mL, preferably 50 mL) and allowed to stir at ambient temperature for 1 to 24 hours (preferably 3 hours). After allotted time the reaction is concentrated and the product is purified by RP-HPLC or silica gel chromatography.

Illustration of General Procedure P

### Example 9

### 4-(2-chlorothieno[3,2-d]pyrimidin-4-yl)-N′-cyano-2-isopropyl-N-(1,2,3,4-tetrahydroisoquinolin-5-yl)piperazine-1-carboximidamide

(9H-fluoren-9-yl)methyl 5-(4-(2-chlorothieno[3,2-d]pyrimidin-4-yl)-N′-cyano-2-isopropylpiperazine-1-carboximidamido)-3,4-dihydroisoquinoline-2-1H-carboxylate (320 mg, 0.45 mmol) was dissolved in 20% (v/v) piperidine in DMF at ambient temperature and allowed to stir for 2 hours at which time it was concentrated and purified by silica gel chromatography employing 7/3 ethylacetate:methanol as the eluent yielding 4-(2-chlorothieno[3,2-d]pyrimidin-4-yl)-N′-cyano-2-isopropyl-N-(1,2,3,4-tetrahydroisoquinolin-5-yl)piperazine-1-carboximidamide (41 mg, 0.08 mmol) as a white solid.

Rₜ 4.95 min (method e)

MS m/z: (M+H)⁺ 495

General Procedure Q: Arylamide Formation by Palladium-Catalyzed Carbonylation

The 6′-chloro-3-isopropyl-3,4,5,6-tetrahydro-2H-[1,2′]bipyrazinyl and 3′-chloro-3-isopropyl-3,4,5,6-tetrahydro-2H-[1,2′]bipyrazinyl are prepared from isopropylpiperazine and 2,3-dichloropyrazine or 2,5-dichloropyrazing following general procedure H. The 2- or 3-chloro isopropylpiperazine-pyrazine, palladium catalyst (such as palladiumdichloride ditriphenyl phosphine, palladium tetrakistriphenylphosphine or palladium dichloride diphenylphosphinoferrocene, preferably palladiumdichloride ditriphenyl phosphine, 0.05 to 0.2 equivalents, preferably 0.1 equivalents), primary or secondary amine (1.0 to 5.0 equivalents, preferably 3.0 equivalents) and tertiary amine (triethyl amine, diisopropylethyl amine, preferably diisopropylethyl amine, 1 to 4 equivalents, preferably 3 equivalents) are dissolved in an organic solvent (for example tetrahydrofuran, dimethylformamide, dioxane, preferably dimethylformamide) at ambient temperature. A balloon of carbon monoxide is attached and the head space is evacuated and re-filled with carbon monoxide 1 to 3 times (preferably 3). The reaction is then heated 50-100° C. (preferably 100° C.) for 2-24 hours (preferably 16 hours). The product is concentrated and purified by reverse phase HPLC.

Illustration of General Procedure Q

### Example 10

### 3-Isopropyl-3,4,5,6-tetrahydro-2H-[1,2′]bipyrazinyl-6′-carboxylic acid dimethylamide

Dissolved 6′-Chloro-3-isopropyl-3,4,5,6-tetrahydro-2H-[1,2′]bipyrazinyl (500 mg, 2.0 mmol), dichloropalladium ditriphenylphosphine (170 mg, 0.2 mmol) and dimethylamine hydrochloride (510 mg, 6.25 mmol) in 10 mL of DMF. To this solution was added triethylamine (1.6 mL, 11.45 mmol) and a balloon filled with carbon monoxide. The reaction was vacuum purged 3 times with carbon monoxide and then heated to 100° C. and allowed to stir for 16 h after which time it was cooled and concentrated under reduced pressure. The crude material was brought up in dichloromethane and filtered through a plug of silica gel to provide 260 mg (0.94 mmol, 47% yield) of 3-isopropyl-3,4,5,6-tetrahydro-2H-[1,2′]bipyrazinyl-6′-carboxylic acid dimethylamide as an oil which was used in subsequent reactions without further purification.

RP-HPLC (Method e) Rₜ 6.0 min; MS m/z: (M+H)⁺ 278.

### Individual Examples

### Example 11

### Preparation of N-(4-chlorophenyl)-4-(2-cyano-3-fluorophenyl)-2-phenylpiperazine-1-carboxamide

2-Phenylpiperazine (162 mg, 1 mmol), 2,6-difluorobenzonitrile (139 mg, 1 mmol), and potassium carbonate (138 mg, 1 mmol) in DMSO (1 mL) were heated to 100° C. for 2.5 hours. The mixture was cooled to ambient temperature, 1-chloro-4-isocyanatobenzene (154 mg, 1 mmol) was added, and after 30 min., the product was purified by reverse-phase HPLC on a Hyperprep HS C18 column, 8 μm, 250×21.2 mm; 20% acetonitrile-50 mM ammonium acetate over 1 min, 20-100% acetonitrile-50 mM ammonium acetate over 24 min, 100% acetonitrile for 5 min, 20 mL/min. The product was isolated by extraction of the desired fractions with DCM. The combined organic phase was dried with sodium sulfate and concentrated to give a yellow foam of N-(4-chlorophenyl)-4-(2-cyano-3-fluorophenyl)-2-phenylpiperazine-1-carboxamide (217 mg, 0.50 mmol).

Retention time: 3.41 min. (method i), m/z: (M−H)⁻ 433.

### Example 12

### Preparation of 5-(N′-cyano-4-(3,4-dimethoxybenzoyl)-3-phenylpiperazine-1-carboximidamido)-1-methylquinolinium acetate

Methyl iodide (142 mg, 1 mmol) was added to a solution of N′-cyano-4-(3,4-dimethoxybenzoyl)-3-phenyl-N-(quinolin-5-yl)piperazine-1-carboximidamide (5 mg, 0.01 mmol) in MeCN (1 mL). After stirring at ambient temperature for 16 h, the product was purified by reverse-phase HPLC on a Hyperprep HS C18 column, 8 μm, 250×21.2 mm; 20% acetonitrile-50 mM ammonium acetate over 1 min, 20-60% acetonitrile-50 mM ammonium acetate over 29 min, 60-100% acetonitrile over 1 min, 100% acetonitrile for 4 min, 20 mL/min. The product was isolated by lyophilization of the desired fractions to give a dark violet solid of 5-(N′-cyano-4-(3,4-dimethoxybenzoyl)-3-phenylpiperazine-1-carboximidamido)-1-methylquinolinium acetate (3.2 mg, 0.006 mmol).

Retention time: 1.22 min. (method d), m/z: (M+H)⁺ 535.

### Example 13

### Preparation of 4-(2-chloropyrimidin-4-yl)-N′-cyano-3-phenyl-N-o-tolylpiperazine-1-carboximidamide

4-(2-chloropyrimidin-4-yl)-N′-cyano-3-phenyl-N-o-tolylpiperazine-1-carboximidamide was prepared from N′-Cyano-3-phenyl-N-o-tolylpiperazine-1-carboximidamide and 2,4-dichloropyrimidine using general procedure H.

Retention time: 1.83 min. (method d), m/z: (M+H)⁺ 432.

### Example 14

### Preparation of 4-(2-chloropyrimidin-4-yl)-N′-cyano-2-isopropyl-N-(1,2,3,4-tetrahydroisoquinolin-5-yl)piperazine-1-carboximidamide

### a) 2-Chloro-4-(3-isopropylpiperazin-1-yl)pyrimidine

2-Chloro-4-(3-isopropylpiperazin-1-yl)pyrimidine was prepared from 2-isopropylpiperazine and 2,4-dichloropyrimidine using general procedure H.

m/z: (M+H)⁺ 241.

### b) (9H-Fluoren-9-yl)methyl 5-(4-(2-chloropyrimidin-4-yl)-N′-cyano-2-isopropylpiperazine-1-carboximidamido)-3,4-dihydroisoquinoline-2(1H)-carboxylate

(9H-Fluoren-9-yl)methyl 5-(4-(2-chloropyrimidin-4-yl)-N′-cyano-2-isopropylpiperazine-1-carboximidamido)-3,4-dihydroisoquinoline-2(1H)-carboxylate was prepared from 2-chloro-4-(3-isopropylpiperazin-1-yl)pyrimidine and 5-isothiocyanato-3,4-dihydro-1H-isoquinoline-2-carboxylic acid 9H-fluoren-9-ylmethyl ester according to general procedure G.

m/z: (M−H)⁻ 659

### c) 4-(2-chloropyrimidin-4-yl)-N′-cyano-2-isopropyl-N-(1,2,3,4-tetrahydroisoquinolin-5-yl)piperazine-1-carboximidamide

4-(2-chloropyrimidin-4-yl)-N′-cyano-2-isopropyl-N-(1,2,3,4-tetrahydroisoquinolin-5-yl)piperazine-1-carboximidamide was prepared from (9H-Fluoren-9-yl)methyl 5-(4-(2-chloropyrimidin-4-yl)-N′-cyano-2-isopropylpiperazine-1-carboximidamido)-3,4-dihydroisoquinoline-2(1H)-carboxylate according to general procedure P.

Retention time: 1.33 min. (method d), m/z: (M+H)⁺ 439.

### Example 15

### Preparation of 4-(N′-cyano-N-(3-(2-(dimethylamino)acetamido)-2-methylphenyl)carbanimidoyl)-N-(3-fluorophenyl)-3-isopropylpiperazine-1-carboxamide acetate

### a) 3-Isopropylpiperazin-1-carboxylic acid (3-fluorophenyl)amide

3-Isopropylpiperazin-1-carboxylic acid (3-fluorophenyl)amide was prepared from 2-isopropylpiperazine and 1-fluoro-3-isocyanatobenzene according to general procedure C.

m/z: (M−H)⁻ 264

### b) 4-(N′-Cyano-N-(2-methyl-3-nitrophenyl)carbanimidoyl)-N-(3-fluorophenyl)-3-isopropylpiperazine-1-carboxamide

4-(N′-Cyano-N-(2-methyl-3-nitrophenyl)carbamimidoyl)-N-(3-fluorophenyl)-3-isopropylpiperazine-1-carboxamide was prepared from 3-isopropylpiperazin-1-carboxylic acid (3-fluorophenyl)amide and 1-isocyanato-2-methyl-3-nitrobenzene according to general procedure G. m/z: (M+H)⁺ 468.

### c) 4-(N-(3-Amino-2-methylphenyl)-N′-cyanocarbamimidoyl)-N-(3-fluorophenyl)-3-isopropylpiperazine-1-carboxamide

4-(N′-Cyano-N-(2-methyl-3-nitrophenyl)carbamimidoyl)-N-(3-fluorophenyl)-3-isopropylpiperazine-1-carboxamide (1.28 g, 2.73 mmol) was dissolved in MeOH (50 mL), palladium on carbon (500 mg) was added, and the suspension was hydrogenated during 16 hours in a Parr hydrogenator under a hydrogen pressure of 60 psi. The catalyst was removed by filtration and the filtrate was concentrated to give 4-(N-(3-Amino-2-methylphenyl)-N′-cyanocarbamimidoyl)-N-(3-fluorophenyl)-3-isopropylpiperazine-1-carboxamide (624 mg, 1.43 mmol). m/z: (M+H)⁺ 438.

### d) 4-(N′-cyano-N-(3-(2-(dimethylamino)acetamido)-2-methylphenyl)carbamimidoyl)-N-(3-fluorophenyl)-3-isopropylpiperazine-1-carboxamide acetate

4-(N′-cyano-N-(3-(2-(dimethylamno)acetamido)-2-methylphenyl)carbamimdoyl)-N-(3-fluorophenyl)-3-isopropylpiperazine-1-carboxamide acetate was prepared from 4-(N-(3-Amino-2-methylphenyl)-N′-cyanocarbamimidoyl)-N-(3-fluorophenyl)-3-isopropylpiperazine-1-carboxamide and dimethylaminoacetyl chloride according to general procedure D.

Retention time: 1.63 min. (method b), m/z: (M+H)⁺ 523.

### Example 16

### Preparation of 4-(2-chloropyrimidin-4-yl)-N′-cyano-2-isopropyl-N-(1,2,3,4-tetrahydroquinolin-5-yl)piperazine-1-carboximidamide

4-(2-chloropyrimidin-4-yl)-N′-cyano-2-isopropyl-N-(1,2,3,4-tetrahydroquinolin-5-yl)piperazine-1-carboximidamide was prepared from 2-chloro-4-(3-isopropylpiperazin-1-yl)pyrimidine and 5-isothiocyanato-1,2,3,4-tetrahydroquinoline according to general procedure G.

Retention time: 1.70 min. (method d), m/z: (M+H)⁺ 439.

### Example 17

### Preparation of 4-(benzo[d]oxazol-2-yl)-N′-cyano-3-phenyl-N-o-tolylpiperazine-1-carboximidamide

4-(benzo[d]oxazol-2-yl)-N′-cyano-3-phenyl-N-o-tolylpiperazine-1-carboximidamide was prepared from N′-Cyano-3-phenyl-N-o-tolylpiperazine-1-carboximidamide and 2-chlorobenzooxazole according general procedure H.

Retention time: 1.93 min. (method d), m/z: (M+H)⁺ 437.

### Example 18

### Preparation of methyl 5-(4-(N′-cyano-N-o-tolylcarbamimidoyl)-2-phenylpiperazin-1-yl)pyrazine-2-carboxylate acetate

methyl 5-(4-(N′-cyano-N-o-tolylcarbamimidoyl)-2-phenylpiperazin-1-yl)pyrazine-2-carboxylate acetate was prepared from N′-Cyano-3-phenyl-N-o-tolylpiperazine-1-carboximidamide and 5-chloropyrazine-2-carboxylic acid methyl ester according general procedure H.

Retention time: 1.72 min. (method d), m/z: (M+H)⁺ 456.

### Example 19

### Preparation of N′-cyano-4-(imidazo[1,2-b]pyridazin-6-yl)-2-isopropyl-N-(2-methylquinolin-5-yl)piperazine-1-carboximidamide acetate

### a) 6-(3-Isopropylpiperazin-1-yl)imidazo[1,2-b]pyridazine

6-(3-Isopropylpiperazin-1-yl)imidazo[1,2-b]pyridazine was prepared from 6-chloroimidazo[1,2-b]pyridazine and 2-isopropylpiperazine according to general procedure H. m/z: (M+H)⁺ 246.

### b) N′-cyano-4-(imidazo[1,2-b]pyridazin-6-yl)-2-isopropyl-N-(2-methylquinolin-5-yl)piperazine-1-carboximidamide acetate

N′-cyano-4-(imidazo[1,2-b]pyridazin-6-yl)-2-isopropyl-N-(2-methylquinolin-5-yl)piperazine-1-carboximidamide acetate was prepared from 6-(3-Isopropylpiperazin-1-yl)imidazo[1,2-b]pyridazine and 5-isothiocyanato-2-methylquinoline.

Retention time: 1.75 min. (method b), m/z: (M+H)⁺ 454.

### Examples 20 and 21

### Preparation of N′-cyano-2-isopropyl-4-(2-oxo-1,2-dihydropyrimidin-4-yl)-N-(quinolin-5-yl)piperazine-1-carboximidamide and 2-isopropyl-4-(2-oxo-1,2-dihydropyrimidin-4-yl)-N-(quinolin-5-yl)piperazine-1-carbothioamide

### a) 6-Chloro-1H-pyrimidin-2-one

To the solution of 2,4-dichloropyrimidine (0.56 g, 3.75 mmol) in dioxane (2 mL), the solution of sodium hydroxide (0.15 g, 3.75 mmol) in water (8 mL) was added and the resulting mixture was stirred at ambient temperature for 18 hours. To this solution, 10 mL of dioxane was added and the resulting precipitate was collected by filtration and dried to yield 6-chloro-1H-pyrimidin-2-one (0.09 g, 0.7 mmol) as a white solid.

m/z: (M−H)⁻ 129.

### b) 6-(3-isopropyl-piperazin-1-yl)-1H-pyrimidin-2-one

6-(3-isopropyl-piperazin-1-yl)-1H-pyrimidin-2-one was prepared from 6-chloro-1H-pyrimidin-2-one and 2-isopropylpiperazine using general procedure H. m/z: (M+H)⁺ 223.

### c) N′-cyano-2-isopropyl-4-(2-oxo-1,2-dihydropyrimidin-4-yl)-N-(quinolin-5-yl)piperazine-1-carboximidamide and 2-isopropyl-4-(2-oxo-1,2-dihydropyrimidin-4-yl)-N-(quinolin-5-yl)piperazine-1-carbothioamide

N′-cyano-2-isopropyl-4-(2-oxo-1,2-dihydropyrimidin-4-yl)-N-(quinolin-5-yl)piperazine-1-carboximidamide and 2-isopropyl-4-(2-oxo-1,2-dihydropyrimidin-4-yl)-N-(quinolin-5-yl)piperazine-1-carbothioamide were prepared from 6-(3-isopropyl-piperazin-1-yl)-1H-pyrimidin-2-one and quinoline-5-isothiocyanate using general procedure G. N′-Cyano-2-isopropyl-4-(2-oxo-1,2-dihydropyrimidin-4-yl)-N-(quinolin-5-yl)piperazine-1-carboximidamide:

Retention time: 3.67 min. (method h), m/z: (M+H)⁺ 417.
2-Isopropyl-4-(2-oxo-1,2-dihydropyrimidin-4-yl)-N-(quinolin-5-yl)piperazine-1-carbothioamide

Retention time: 4.27 min. (method h), m/z: (M−H)⁻ 407.

### Example 22

### Preparation of N-(4-chlorophenyl)-2-phenyl-4-(pyridin-2-ylcarbamothioyl)piperazine-1-carboxamide

### a) 3-Phenylpiperazine-1-carbothioic acid pyridin-3-ylamide

3-Phenylpiperazine-1-carbothioic acid pyridin-3-ylamide was prepared from 3-isothicyanatopyridine and 2-phenylpiperazine using general procedure N.

m/z: (M+H)⁺ 299.

### b) N-(4-chlorophenyl)-2-phenyl-4-(pyridin-2-ylcarbamothioyl)piperazine-1-carboxamide

N-(4-chlorophenyl)-2-phenyl-4-(pyridin-2-ylcarbamothioyl)piperazine-1-carboxamide was prepared from 3-phenylpiperazine-1-carbothioic acid pyridin-3-ylamide and 4-chlorophenylisocyanate using general procedure C.

Retention time: 2.23 min. (method d), m/z: (M−H)⁻ 450.

### Example 23

### Preparation of 4-(benzo[d]oxazol-2-yl)-N-(4-chlorophenyl)-2-phenylpiperazine-1-carboxamide

### a) 2-(3-Phenyl-piperazin-1-yl)-benzooxazole

2-(3-Phenylpiperazin-1-yl)-benzooxazole was prepared from 2-chlorobenzooxazole and 2-phenylpiperazine using general procedure H.

m/z: (M+H)⁺ 246.

### b) 4-(benzo[d]oxazol-2-yl)-N-(4-chlorophenyl)-2-phenylpiperazine-1-carboxamide

4-(benzo[d]oxazol-2-yl)-N-(4-chlorophenyl)-2-phenylpiperazine-1-carboxamide was prepared from 2-(3-Phenyl-piperazin-1-yl)-benzooxazole and 4-chlorophenylisocyanate using general procedure C.

Retention time: 6.02 min. (method h), m/z: (M+H)⁺ 433.

### Example 24

### 4-(1,1-Dioxo-1H-1-λ-6-benzo[d]isothiazol-3-yl)-2-phenylpiperazine-1-carboxylic acid (4-chlorophenyl)amide

### a) 2-Phenyl-piperazine-1-carboxylic acid (4-chloro-phenyl)-amide

2-Phenylpiperazine-1-carboxylic acid (4-chlorophenyl)amide was prepared from 2-phenylpiperazine and 4-chlorophenylisocyanate using general procedure C.

m/z: (M+H)⁺ 316.

### b) 4-(1,1-Dioxo-1H-1-λ-6-benzo[d]isothiazol-3-yl)-2-phenylpiperazine-1-carboxylic acid (4-chlorophenyl)amide

4-(1,1-Dioxo-1H-1-λ-6-benzo[d]isothiazol-3-yl)-2-phenylpiperazine-1-carboxylic acid (4-chlorophenyl)amide was prepared from 3-chlorobenzo[d]isothiazole 1,1-dioxide and 2-phenylpiperazine-1-carboxylic acid (4-chlorophenyl)amide using general procedure H.

Retention time: 2.01 min. (method d), m/z: (M−H)⁻ 479.

### Example 25

### Preparation of trans-N-(4-chlorophenyl)-4-(N′-cyano-N-o-tolylcarbamimidoyl) octahydroquinoxaline-1(2H)-carboxamide

### a) Trans- decahydroquinoxaline and cis-decahydroquinoxaline

Quinoxaline (3.6 g, 27.7 mmol) was dissolved in ethanol (150 mL), 10% palladium on carbon (0.6 g) was added and the mixture was hydrogenated on the Parr shaker apparatus at 60 psi for 120 hours. The catalyst was removed by filtration; the solvent was removed under reduced pressure and the residue triturated in ether (175 mL) and was left standing in the refrigerator for 24 hours. The precipitate was collected by filtration and dried to yield analytically pure trans-decahydroquinoxaline (1.9 g, 14.2 mmol).

m/z: (M+H)⁺: 141.

The filtrate was concentrated to yield cis-decahydroquinoxaline (1.8 g, 13.5 mmol) in 90% purity. m/z: (M+H)⁺: 141.

### b) Trans-4-(N′-cyano-N-o-tolylcarbamimidoyl)decahydroquinoxaline

Trans-4-(N′-cyano-N-o-tolylcarbamimidoyl)decahydroquinoxaline was prepared from trans-decahydroquinoxaline and N-cyano-N′-(2-methylphenyl)carbamimidic acid phenyl ester using general procedure B. m/z: (M+H)⁺: 298.

### c) Trans-N-(4-chlorophenyl)-4-(N′-cyano-N-o-tolylcarbamimidoyl)octahydroquinoxaline-1(2H)-carboxamide

Trans- N-(4-chlorophenyl)-4-(N′-cyano-N-o-tolylcarbamimidoyl)octahydroquinoxaline-1(2H)-carboxamide was prepared from trans-4-(N′-cyano-N-o-tolylcarbamimidoyl)decahydroquinoxaline and 4-chlorophenylisocyanate using general procedure C.

Retention time: 5.77 min. (method h), m/z: (M+H)⁺ 451

### Example 26

### Preparation of cis-N-(4-chlorophenyl)-4-(N′-cyano-N-o-tolylcarbamimidoyl)octahydroquinoxaline-1(2H)-carboxamide

### a) Cis-4-(N′-cyano-N-o-tolylcarbamimidoyl)decahydroquinoxaline

Cis-4-(N′-cyano-N-o-tolylcarbamimidoyl)decahydroquinoxaline was prepared from cis-decahydroquinoxaline and N-cyano-N′-(2-methylphenyl)carbamimidic acid phenyl ester using general procedure B.

m/z: (M+H)⁺: 298.

### b) Cis-N-(4-chlorophenyl)-4-(N′-cyano-N-o-tolylcarbamimidoyl)octahydroquinoxaline-1(2H)-carboxamide

Cis- N-(4-chlorophenyl)-4-(N′-cyano-N-o-tolylcarbamimidoyl)octahydroquinoxaline-1(2H)-carboxamide was prepared from cis-4-(N′-cyano-N-o-tolylcarbamimidoyl)decahydroquinoxaline and 4-chlorophenylisocyanate using general procedure C.

Retention time: 1.95 min. (method d), m/z: (M+H)⁺ 451

### Example 27

### Preparation of N′-cyano-2-isopropyl-4-(pyridazin-3-yl)-N-(quinolin-5-yl)piperazine-1-carboximidamide

### a) 3-Chloro-6-(3-isopropylpiperazin-1-yl)-pyridazine

3-Chloro-6-(3-isopropylpiperazin-1-yl)-pyridazine was prepared from 2-isopropylpiperazine and 3,6-dichloropyridazine using general procedure H.

m/z: (M+H)⁺ 241.

### b) 3-(3-Isopropylpiperazin-1-yl)-pyridazine

3-Chloro-6-(3-isopropylpiperazin-1-yl)-pyridazine (0.24 g, 1.0 mmol) and ammonium formate (0.31 g, 5.0 mmol) were triturated in methanol (20 mL), 10% palladium on carbon (0.1 g) was added and the reaction mixture was heated at 50° C. for 2 hours. The catalyst was removed by filtration; the filtrate was concentrated under reduced pressure and the residue triturated in EtOAc. The insoluble residue was removed by filtration and the filtrate concentrated to yield 3-(3-Isopropylpiperazin-1-yl)-pyridazine (0.19 g, 0.92 mmol) as an off-white solid.

m/z: (M+H)⁺ 207.

### c) N′-cyano-2-isopropyl-4-(pyridazin-3-yl)-N-(quinolin-5-yl)piperazine-1-carboximidamide

N′-cyano-2-isopropyl-4-(pyridazin-3-yl)-N-(quinolin-5-yl)piperazine-1-carboximidamide was prepared from 3-(3-Isopropylpiperazin-1-yl)-pyridazine and quinoline-5-isothiocyanate using general procedure G.

Retention time: 1.21 min. (method d), m/z: (M−H)⁻ 399.

### Example 28

### Preparation of N′-cyano-2-isopropyl-4-(pyrimidin-4-yl)-N-(quinolin-5-yl)piperazine-1-carboximidamide

### a) 2-Chloro-4-(3-isopropylpiperazin-1-yl)-pyrimidine

2-Chloro-4-(3-isopropylpiperazin-1-yl)-pyrimidine was prepared from 2-isopropylpiperazine and 2,4-dichloropyrimidine using general procedure H.

m/z: (M+H)⁺ 241.

### b) 4-(3-Isopropyl-piperazin-1-yl)-pyrimidine

2-Chloro-4-(3-isopropylpiperazin-1-yl)-pyrimidine (0.48 g, 2.0 mmol) and ammonium formate (0.63 g, 10.0 mmol) were triturated in methanol (40 mL), 10% palladium on carbon (0.2 g) was added and the reaction mixture was heated at 50° C. for 2 hours. The catalyst was removed by filtration; the filtrate was concentrated under reduced pressure and the residue triturated in EtOAc. The insoluble residue was removed by filtration and the filtrate concentrated to yield 4-(3-isopropyl-piperazin-1-yl)-pyrimidine (0.37 g, 1.79 mmol) as a yellow solid.

m/z: (M+H)⁺ 207.

### c) N′-cyano-2-isopropyl-4-(pyrimidin-4-yl)-N-(quinolin-5-yl)piperazine-1-carboximidamide

N′-cyano-2-isopropyl-4-(pyrimidin-4-yl)-N-(quinolin-5-yl)piperazine-1-carboximidamide was prepared from 4-(3-isopropyl-piperazin-1-yl)-pyrimidine and quinoline 5-isothiocyanate using general procedure G.

Retention time: 1.49 min. (method d), m/z: (M+H)⁺ 401.

### Example 29

### Preparation of N′-cyano-4-(6-(2-(dimethylamino)ethylamino)pyrimidin-4-yl)-2-isopropyl-N-(quinolin-5-yl)piperazine-1-carboximidamide diacetate

### a) 4-Chloro-6-(3-isopropylpiperazin-1-yl)-pyrimidine

4-Chloro-6-(3-isopropylpiperazin-1-yl)-pyrimidine was prepared from 4,6-dichloropyrimidine and 2-isopropylpiperazine using general procedure H.

m/z: (M+H)⁺ 241

### b) N′-[6-(3-Isopropylpiperazin-1-yl)-pyrimidin-4-yl]-N,N-dimethyl-ethane -1,2-diamine

N′-[6-(3-Isopropylpiperazin-1-yl)-pyrimidin-4-yl]-N,N-dimethyl-ethane -1,2-diamine was prepared from N,N-dimethylethylenediamine and 4-chloro-6-(3-isopropylpiperazin-1-yl)-pyrimidine using general procedure H.

m/z: (M+H)⁺ 293

### c) N′-cyano-4-(6-(2-(dimethylamino)ethylamino)pyrimidin-4-yl)-2-isopropyl-N-(quinolin-5-yl)piperazine-1-carboximidamide diacetate

N′-cyano-4-(6-(2-(dimethylamino)ethylamino)pyrimidin-4-yl)-2-isopropyl-N-(quinolin-5-yl)piperazine-1-carboximidamide diacetate was prepared from N′-[6-(3-isopropylpiperazin-1-yl)-pyrimidin-4-yl]-N,N-dimethyl-ethane -1,2-diamine and quinoline 5-isothiocyanate using general procedure G.

Retention time: 1.17 min. (method d), m/z: (M+H)⁺ 487.

### Example 30

### Preparation of 4-(6-(2-(dimethylamino)ethylamino)pyrimidin-4-yl)-2-isopropyl-N-(quinolin-5-yl)piperazine-1-carbothioamide diacetate

4-(6-(2-(Dimethylamino)ethylamino)pyrimidin-4-yl)-2-isopropyl-N-(quinolin-5-yl)piperazine-1-carbothioamide diacetate was prepared from N′-[6-(3-isopropylpiperazin-1-yl)-pyrimidin-4-yl]-N,N-dimethyl-ethane -1,2-diamine and quinoline 5-isothiocyanate using general procedure G.

Retention time: 1.47 min. (method d), m/z: (M−H)⁻ 477.

### Example 31

### Preparation of N′-cyano-2-phenyl-4-(6-phenylpyridazin-3-yl)-N-o-tolylpiperazine-1-carboximidamide

### a) 3-Phenyl-6-(3-phenylpiperazin-1-yl)-pyridazine

3-Phenyl-6-(3-phenylpiperazin-1-yl)-pyridazine was prepared from 3-chloro-6-phenylpyridazine and 2-phenylpiperazine using general procedure H.

m/z: (M+H)⁺ 317.

### b) N′-cyano-2-phenyl-4-(6-phenylpyridazin-3-yl)-N-o-tolylpiperazine-1-carboximidamide

N′-cyano-2-phenyl-4-(6-phenylpyridazin-3-yl)-N-o-tolylpiperazine-1-carboximidamide was prepared from 3-phenyl-6-(3-phenylpiperazin-1-yl)-pyridazine and N-cyano-N′-(2-methylphenyl)carbamimidic acid phenyl ester using general procedure B.

Retention time: 3.5 min. (method a), m/z: (M−H)⁻ 472.

### Example 32

### Preparation of 4-(2-chloropyrimidin-4-yl)-N′-cyano-2-phenyl-N-o-tolylpiperazine-1-carboximidamide

### a) 2-Chloro-4-(3-phenylpiperazin-1-yl)-pyrimidine

2-Chloro-4-(3-phenylpiperazin-1-yl)-pyrimidine was prepared from 2,4-dichloropyrimidine and 2-phenylpiperazine using general procedure H.

m/z: (M+H)⁺ 275.

### b) 4-(2-chloropyrimidin-4-yl)-N′-cyano-2-phenyl-N-o-tolylpiperazine-1-carboximidamide

4-(2-Chloropyrimidin-4-yl)-A-cyano-2-phenyl-N-o-tolylpiperazine-1-carboximidamide was prepared from 2-chloro-4-(3-phenylpiperazin-1-yl)-pyrimidine and N-cyano-N′-(2-methylphenyl)carbamimidic acid phenyl ester using general procedure B.

Retention time: 1.9 min. (method a), m/z: (M−H)⁻ 430.

### Preparation 1: Preparation of 4-chloro-2-(3-isopropylpiperazin-1-yl)-quinazoline

### a) 2-(3-Isopropylpiperazin-1-yl)-quinazolin-4-ol

2-(3-Isopropylpiperazin-1-yl)quinazolin-4-ol was prepared from 2-chloro-quinazolin-4-ol and 2-isopropylpiperazine using general procedure H.

m/z: (M+H)⁺ 273.

### b) 4-Chloro-2-(3-isopropylpiperazin-1-yl)-quinazoline

2-(3-Isopropylpiperazin-1-yl)quinazolin-4-ol (0.17 g, 0.63 mmol) was suspended in phosphorus oxychloride (1.5 mL) and the reaction mixture was heated at 100° C. for 2 hours. The reaction mixture was cooled to ambient temperature and added dropwise into a saturated solution of sodium hydrogen carbonate (30 mL). The organic phase was extracted with EtOAc (2×35 mL); the combined organic extracts dried with magnesium sulfate and concentrated to yield 4-chloro-2-(3-isopropylpiperazin-1-yl)-quinazoline (0.15 g, 0.52 mmol) as an off-white solid.

m/z: (M+H)⁺ 291.

### Example 33

### Preparation of N′-cyano-2-isopropyl-4-(4-oxo-3,4-dihydroquinazolin-2-yl)-N-(quinolin-5-yl)piperazine-1-carboximidamide

N′-cyano-2-isopropyl-4-(4-oxo-3,4-dihydroquinazolin-2-yl)-N-(quinolin-5-yl)piperazine-1-carboximidamide was prepared from 2-(3-isopropylpiperazin-1-yl)quinazolin-4-ol and quinoline-5-isothiocyanate using general procedure Gtemp.

Retention time: 2.12 min. (method d), m/z: (M+H)⁺ 467.

### Example 34

### Preparation of N′-cyano-2-isopropyl-4-(quinazolin-2-yl)-N-o-tolylpiperazine-1-carboximidamide

### a) 2-(3-Isopropylpiperazin-1-yl)quinazoline

4-Chloro-2-(3-isopropylpiperazin-1-yl)-quinazoline (0.5 g, 1.72 mmol) was dissolved in EtOAc (50 mL), 10% palladium on carbon (0.1 g) was added and the mixture was hydrogenated on the Parr shaker apparatus at 60 psi for 48 hours. The catalyst was removed by filtration; the solvent was removed under reduced pressure to yield 2-(3-isopropylpiperazin-1-yl)quinazoline (0.43 g, 1.7 mmol) as a yellow solid.

m/z: (M+H)⁺ 257.

### b) N′-cyano-2-isopropyl-4-(quinazolin-2-yl)-N-o-tolylpiperazine-1-carboximidamide

N′-cyano-2-isopropyl-4-(quinazolin-2-yl)-N-o-tolylpiperazine-1-carboximidamide was prepared from 2-(3-isopropylpiperazin-1-yl)quinazoline and N-cyano-N′-(2-methylphenyl)carbamimidic acid phenyl ester using general procedure B.

Retention time: 2.14 min. (method d), m/z: (M+H)⁺ 414.

### Example 35

### Preparation of 4-(6-aminopyridazin-3-yl)-2-isopropyl-N-(quinolin-5-yl)piperazine-1-carbothioamide acetate

### a) 6-(3-Isopropylpiperazin-1-yl)pyridazin-3-ylamine

6-(3-Isopropylpiperazin-1-yl)pyridazin-3-ylamine was prepared from N-(6-chloro-pyridazin-3-yl)-2,2,2-trifluoro-acetamide and 2-isopropylpiperazine using general procedure H.

m/z: (M+H)⁺ 222.

### b) 4-(6-Aminopyridazin-3-yl)-2-isopropyl-N-(quinolin-5-yl)piperazine-1-carbothioamide acetate

4-(6-Aminopyridazin-3-yl)-2-isopropyl-N-(quinolin-5-yl)piperazine-1-carbothioamide acetate was prepared from 6-(3-isopropylpiperazin-1-yl)pyridazin-3-ylamine and quinoline-5-isothiocyanate using general procedure N.

Retention time: 1.96 min. (method d)

m/z: (M−H)⁻ 406.

### Example 36

### Preparation of 2-isopropyl-4-(6-(isopropylamino)pyridazin-3-yl)-N-(quinolin-5-yl)piperazine-1-carbothioamide

A suspension of 4-(6-aminopyridazin-3-yl)-2-isopropyl-N-(quinolin-5-yl)piperazine-1-carbothioamide (0.1 g, 0.246 mmol), acetone (0.028 g, 0.49 mmol), acetic acid (0.016 g, 0.27 mmol) and sodium triacetoxyborohydride (0.068 g, 0.31 mmol) in dichloroethane (4 mL) was stirred at 40° C. for 48 hours. The solvent was removed under reduced pressure and the residue was subjected to preparative RP-HPLC (20% to 50% acetonitrile/0.05M aqueous ammonium acetate, buffered to pH 4.5, over 30 min at 21 mL/min; λ=254 nm; Microsorb C18, 100 Å, 5 μm, 250×46 mm column) to yield 2-isopropyl-4-(6-(isopropylamino)pyridazin-3-yl)-N-(quinolin-5-yl)piperazine-1-carbothioamide (0.07 g, 0.016 mmol) as an off-white solid.

Retention time: 1.29 min. (method d), m/z: (M−H)⁻ 448.

### Example 37

### Preparation of 2-isopropyl-4-(6-(pyrrolidine-1-carbonyl)pyridazin-3-yl)-N-(quinolin-5-yl)piperazine-1-carbothioamide

### a) [6-(3-Isopropylpiperazin-1-yl)pyridazin-3-yl]pyrrolidin-1-ylmethanone

A solution of 3-chloro-6-(3-isopropylpiperazin-1-yl)pyridazine (0.123 g, 0.51 mmol), pyrrolidine (0.18 g, 2.55 mmol), triethylamine (0.1 g, 1.02 mmol) and palladium chloride DPPF complex with dichloromethane (1:1) (0.041 g, 0.05 mmol) in DMF (10 mL) was heated at 90° C. in the atmosphere of carbon monoxide for 4 hours. The insoluble residue was filtered off and the filtrate was concentrated to yield crude [6-(3-isopropylpiperazin-1-yl)pyridazin-3-yl]pyrrolidin-1-ylmethanone (0.15 g, 0.5 mmol) as a brown solid that was used without further purification.

m/z: (M+H)⁺ 304

### b) 2-Isopropyl-4-(6-(pyrrolidine-1-carbonyl)pyridazin-3-yl)-N-(quinolin-5-yl)piperazine-1-carbothioamide

2-Isopropyl-4-(6-(pyrrolidine-1-carbonyl)pyridazin-3-yl)-N-(quinolin-5-yl)piperazine-1-carbothioamide was prepared from [6-(3-Isopropylpiperazin-1-yl)pyridazin-3-yl]pyrrolidin-1-ylmethanone and quinoline-5-isothiocyanate using general procedure N.

Retention time: 1.47 min. (method d), m/z: (M−H)⁻ 488.

### Examples 38 and 39

### Preparation of N′-cyano-2-isopropyl-4-(6-(morpholine-4-carbonyl)pyridazin-3-yl)-N-(quinolin-5-yl)piperazine-1-carboximidamide and 2-isopropyl-4-(6-(morpholine-4-carbonyl)pyridazin-3-yl)-N-(quinolin-5-yl)piperazine-1-carbothioamide

### a) 2-Isopropyl-4-(6-(morpholine-4-carbonyl)pyridazin-3-yl)-N-(quinolin-5-yl)piperazine-1-carbothioamide

A solution of 3-chloro-6-(3-isopropylpiperazin-1-yl)pyridazine (0.369 g, 0.1.53 mmol), morpholine (0.67 g, 7.65 mmol), triethylamine (0.31 g, 3.06 mmol) and palladium chloride DPPF complex with dichloromethane (1:1) (0.125 g, 0.15 mmol) in DMF (20 mL) was heated at 90° C. in the atmosphere of carbon monoxide for 4 hours. The insoluble residue was filtered off and the filtrate was concentrated to yield crude 2-isopropyl-4-(6-(morpholine-4-carbonyl)pyridazin-3-yl)-N-(quinolin-5-yl)piperazine-1-carbothioamide (0.47 g, 1.46 mmol) as a brown solid that was used without further purification.

m/z: (M+H)⁺ 320

### b) N′-Cyano-2-isopropyl-4-(6-(morpholine-4-carbonyl)pyridazin-3-yl)-N-(quinolin-5-yl)piperazine-1-carboximidamide and 2-isopropyl-4-(6-(morpholine-4-carbonyl)pyridazin-3-yl)-N-(quinolin-5-yl)piperazine-1-carbothioamide

N′-Cyano-2-isopropyl-4-(6-(morpholine-4-carbonyl)pyridazin-3-yl)-N-(quinolin-5-yl)piperazine-1-carboximidamide and 2-isopropyl-4-(6-(morpholine-4-carbonyl)pyridazin-3-yl)-N-(quinolin-5-yl)piperazine-1-carbothioamide were prepared from 2-isopropyl-4-(6-(morpholine-4-carbonyl)pyridazin-3-yl)-N-(quinolin-5-yl)piperazine-1-carbothioamide and quinoline-5-isothiocyanate using general procedure G.
N′-Cyano-2-isopropyl-4-(6-(morpholine-4-carbonyl)pyridazin-3-yl)-N-(quinolin-5-yl)piperazine-1-carboximidamide

Retention time: 1.08 min. (method d), m/z: (M−H)⁻ 512.
2-Isopropyl-4-(6-(morpholine-4-carbonyl)pyridazin-3-yl)-N-(quinolin-5-yl)piperazine-1-carbothioamide

Retention time: 1.3 min. (method d), m/z: (M−H)⁻ 504.

### Example 40

### Preparation of 4-(3-chloroquinoxalin-2-yl)-N-(3-fluorophenyl)-3-phenylpiperazine-1-carboxamide

4-(3-chloroquinoxalin-2-yl)-N-(3-fluorophenyl)-3-phenylpiperazine-1-carboxamide was prepared from 3-phenylpiperazine-1-carboxylic acid (3-fluorophenyl)amide and 2,3-dichloroquinoxaline using general procedure H.

Retention time: 2.67 min. (method d), m/z: (M−H)⁻ 460.

### Example 41

### Preparation of 4-(3-cyanoquinoxalin-2-yl)-N-(3-fluorophenyl)-3-phenylpiperazine-1-carboxamide

A suspension of 4-(3-chloroquinoxalin-2-yl)-N-(3-fluorophenyl)-3-phenylpiperazine-1-carboxamide (0.0.075 g, 0.163 mmol) and sodium cyanide (0.012 g, 0.245 mmol) in DMF (5 mL) was heated at 50° C. for 5 hours. The insoluble residue was filtered off and the solvent was removed under reduced pressure and the residue was subjected to preparative RP-HPLC (40% to 70% acetonitrile/0.05M aqueous ammonium acetate, buffered to pH 4.5, over 30 min at 21 mL/min; x=254 nm; Microsorb C18, 100 Å, 5 μm, 250×46 mm column), to yield 4-(3-cyanoquinoxalin-2-yl)-N-(3-fluorophenyl)-3-phenylpiperazine-1-carboxamide (0.036 g, 0.08 mmol) as an off-white solid.

Retention time: 2.52 min. (method d), m/z: (M−H)⁻ 451.

### Example 42

### Preparation of N-(3-fluorophenyl)-3-phenyl-4-(quinoxalin-2-yl)piperazine-1-carboxamide

To a solution of 4-(3-chloroquinoxalin-2-yl)-N-(3-fluorophenyl)-3-phenylpiperazine-1-carboxamide (0.19 g, 0.39 mmol) in EtOH (45 mL), 10% palladium on carbon (0.05 g) was added and the mixture was hydrogenated on the Parr shaker apparatus at 60 psi for 24 hours. The catalyst was removed by filtration; the solvent was removed under reduced pressure and the residue was subjected to preparative RP-HPLC (30% to 60% acetonitrile/0.05M aqueous ammonium acetate, buffered to pH 4.5, over 30 min at 21 mL/min; λ=254 nm; Microsorb C18, 100 Å, 5 μm, 250×46 mm column) to yield N-(3-fluorophenyl)-3-phenyl-4-(quinoxalin-2-yl)piperazine-1-carboxamide (0.008 g, 0.019 mmol) as an off-white solid.

Retention time: 2.32 min. (method d), m/z: (M+H)⁺ 428.

### Example 43

### Preparation of N-(3-fluorophenyl)-3-isopropyl-4-(4-oxo-3,4-dihydroquinazolin-2-yl)piperazine-1-carboxamide

N-(3-fluorophenyl)-3-isopropyl-4-(4-oxo-3,4-dihydroquinazolin-2-yl)piperazine-1-carboxamide was prepared from 3-phenylpiperazine-1-carboxylic acid (3-fluoro-phenyl)amide and 2-chloro-4-hydroxyquinazoline using general procedure Htemp.

Retention time: 1.7 min. (method d), m/z: (M+H)⁺ 410.

### Example 44

### Preparation of 4-(4-chloroquinazolin-2-yl)-N-(3-fluorophenyl)-3-isopropylpiperazine-1-carboxamide

A suspension of N-(3-fluorophenyl)-3-isopropyl-4-(4-oxo-3,4-dihydroquinazolin-2-yl)piperazine-1-carboxamide (0.6 g, 1.47 mmol) in phosphorus oxychloride (4 mL) was heated at 55oC for 4 hours. The reaction mixture was cooled to ambient temperature and added dropwise into a saturated solution of sodium hydrogen carbonate (15 mL). The organic phase was extracted with EtOAc (2×15 mL); the combined organic extracts dried with magnesium sulfate and concentrated. The residue was subjected to preparative RP-HPLC (50% to 80% acetonitrile/0.05M aqueous ammonium acetate, buffered to pH 4.5, over 30 min at 21 mL/min; λ=254 nm; Microsorb C18, 100 Å, 5 μm, 250×46 mm column) to yield 4-(4-chloroquinazolin-2-yl)-N-(3-fluorophenyl)-3-isopropylpiperazine-1-carboxamide (0.048 g, 0.11 mmol) as an off-white solid.

Retention time: 3.02 min. (method d), m/z: (M−H)⁻ 426.

### Example 45

### Preparation of 4-(4-ethoxyquinazolin-2-yl)-N-(3-fluorophenyl)-3-isopropylpiperazine-1-carboxamide

A suspension of 4-(4-chloroquinazolin-2-yl)-N-(3-fluorophenyl)-3-isopropylpiperazine-1-carboxamide (0.120 g, 0.28 mmol) and sodium cyanide (0.014, 2.86 mmol) in EtOH (25 mL) was heated at 60° C. for 1 hour. The insoluble residue was removed by filtration and the filtrate concentrated under reduced pressure; the residue was subjected to preparative RP-HPLC (50% to 100% acetonitrile/0.05M aqueous ammonium acetate, buffered to pH 4.5, over 30 min at 21 mL/min; λ=254 nm; Microsorb C18, 100 Å, 5 μm, 250×46 mm column) to yield 4-(4-ethoxyquinazolin-2-yl)-N-(3-fluorophenyl)-3-isopropylpiperazine-1-carboxamide (0.034 g, 0.078 mmol) as an off-white solid.

Retention time: 2.99 min. (method d), m/z: (M+H)⁺ 438.

### Example 46

### Preparation of 4-(3,4-dihydroquinazolin-2-yl)-N-(3-fluorophenyl)-3-isopropylpiperazine-1-carboxamide diacetate

To a solution of 4-(4-chloroquinazolin-2-yl)-N-(3-fluorophenyl)-3-isopropylpiperazine-1-carboxamide (0.12 g, 0.28 mmol) in THF (45 mL), 10% palladium on carbon (0.05 g) was added and the mixture was hydrogenated on the Parr shaker apparatus at 60 psi for 24 hours. The catalyst was removed by filtration; the solvent was removed under reduced pressure and the residue was subjected to preparative RP-HPLC (30% to 60% acetonitrile/0.05M aqueous ammonium acetate, buffered to pH 4.5, over 30 min at 21 mL/min; λ=254 nm; Microsorb C18, 100 Å, 5 μm, 250×46 mm column) to yield 4-(3,4-dihydroquinazolin-2-yl)-N-(3-fluorophenyl)-3-isopropylpiperazine-1-carboxamide diacetate (0.054 g, 0.105 mmol) as an off-white solid.

Retention time: 1.83 min. (method d), m/z: (M+H)⁺ 396.

### Example 47

### Preparation of N-(3-fluorophenyl)-2-isopropyl-4-(quinazolin-2-yl)piperazine-1-carboxamide

N-(3-fluorophenyl)-2-isopropyl-4-(quinazolin-2-yl)piperazine-1-carboxamide was prepared from 2-(3-Isopropyl-piperazin-1-yl)-quinazoline and 3-fluorophenylisocyanate using general procedure C.

Retention time: 2.29 min. (method d), m/z: (M+H)⁺ 394.

### Example 48

### Preparation of 6-(4-(N′-cyano-N-(quinolin-5-yl)carbamimidoyl)-3-isopropylpiperazin-1-yl)-N,N-dimethylpyridazine-3-carboxamide

A solution of 4-(6-bromopyridazin-3-yl)-N′-cyano-2-isopropyl-N-(quinolin-5-yl)piperazine-1-carboximidamide (0.33 g, 0.7 mmol), N,N-diisopropylethylamine (0.063 g, 4.9 mmol), dimethylamine hydrochloride (0.285 g, 3.5 mmol) and palladium chloride DPPF complex with dichloromethane (1:1) (0.057 g, 0.07 mmol) in DMF (10 mL) was heated at 90° C. in the atmosphere of carbon monoxide for 4 hours. The solvent was removed under reduced pressure and the residue purified by preparative RP-HPLC (10% to 40% acetonitrile/0.05M aqueous ammonium acetate, buffered to pH 4.5, over 30 min at 21 mL/min; λ=254 nm; Microsorb C18, 100 Å, 5 μm, 250×46 mm column) to yield 6-(4-(1-cyano-N-(quinolin-5-yl)carbamimidoyl)-3-isopropylpiperazin-1-yl)-N,N-dimethylpyridazine-3-carboxamide (0.066 g, 0.14 mmol) as an off-white solid.

Retention time: 1.94 min. (method a), m/z: (M+H)⁺ 472.

### Example 49

### Preparation of N′-cyano-4-(6-(dimethylamino)pyrimidin-4-yl)-2-isopropyl-N-(quinolin-5-yl)piperazine-1-carboximidamide

### a) 4-Chloro-6-(3-isopropylpiperazin-1-yl)pyrimidine

4-Chloro-6-(3-isopropylpiperazin-1-yl)pyrimidine was prepared from 2-isopropylpiperazine and 4,6-dichloropyrimidine following general procedure H.

m/z: (M+H)⁺ 241.

### b) [6-(3-Isopropyl-piperazin-1-yl)pyrimidin-4-yl]dimethylamine

[6-(3-Isopropyl-piperazin-1-yl)pyrimidin-4-yl]dimethylamine was prepared from 4-chloro-6-(3-isopropylpiperazin-1-yl)pyrimidine and dimethylamine solution in methanol following general procedure H.

m/z: (M+H)⁺ 250.

### c) N′-cyano-4-(6-(dimethylamino)pyrimidin-4-yl)-2-isopropyl-N-(quinolin-5-yl)piperazine-1-carboximidamide

N′-cyano-4-(6-(dimethylamino)pyrimidin-4-yl)-2-isopropyl-N-(quinolin-5-yl)piperazine-1-carboximidamide was prepared from [6-(3-isopropyl-piperazin-1-yl)pyrimidin-4-yl]dimethylamine and quinoline-5-isothiocyanate using general procedure Gtemp.

Retention time: 2.12 min. (method a)

m/z: (M+H)⁺ 444.

### Example 50

### Preparation of 5-(4-(N′-cyano-N-(quinolin-5-yl)carbamimidoyl)-3-isopropylpiperazin-1-yl)pyrazine-2-carboxylic acid

### a) 3-Isopropyl-3,4,5,6-tetrahydro-2H-[1,2′]bipyrazinyl-5′-carboxylic acid methyl ester

3-Isopropyl-3,4,5,6-tetrahydro-2H-[1,2′]bipyrazinyl-5′-carboxylic acid methyl ester was prepared from isopropylpiperazine and 5-Chloro-pyrazine-2-carboxylic acid methyl ester following general procedure H.

m/z: (M+H)⁺ 265

### b) 5-(4-(N′-cyano-N-(quinolin-5-yl)carbaminidoyl)-3-isopropylpiperazin-1-yl)pyrazine-2-carboxylic methyl ester

5-(4-(1-cyano-N-(quinolin-5-yl)carbamimidoyl)-3-isopropylpiperazin-1-yl)pyrazine-2-carboxylic acid was prepared from 3-Isopropyl-3,4,5,6-tetrahydro-2H-[1,2′]bipyrazinyl-5′-carboxylic acid methyl ester and 5-Isothiocyanato-quinoline following general procedure G.

m/z: (M+H)⁺ 459

### c) 5-(4-(N′-cyano-N-(quinolin-5-yl)carbamimidoyl)-3-isopropylpiperazin-1-yl)pyrazine-2-carboxylic acid

To a solution of 5-(4-(N′-cyano-N-(quinolin-5-yl)carbaminidoyl)-3-isopropylpiperazin-1-yl)pyrazine-2-carboxylic methyl ester (90 mg, 0.2 mmol) in dioxane (3 mL) was added lithium hydroxide (17 mg, 40 mmol) in water (5 mL) at ambient temperature. The reaction was allowed to stir for 2 hours upon which time it was concentrated and purified by HPLC to give 80 mg (0.2 mmol) of the title compound.
5-(4-(N′-cyano-N-(quinolin-5-yl)carbamimidoyl)-3-isopropylpiperazin-1-yl)pyrazine-2-carboxylic acid

Retention time: 1.92 min (method d)

m/z: (M+H)⁺ 445.

### Examples 51 and 52

### Preparation of 5-(3-isopropyl-4-(quinolin-5-ylcarbamothioyl)piperazin-1-yl)-N-methylpyrazine-2-carboxamide, 5-(3-isopropyl-4-(quinolin-5-ylcarbamothioyl)piperazin-1-yl)-N,N-dimethylpyrazine-2-carboxamide and N-(2-aminoethyl)-5-(3-isopropyl-4-(quinolin-5-ylcarbamothioyl)piperazine-1-yl)pyrazine-2-carboxamide

### a) 3-Isopropyl-3,4,5,6-tetrahydro-2H-[1,2′]bipyrazinyl-5′-carboxylic acid methyl ester

3-Isopropyl-3,4,5,6-tetrahydro-2H-[1,2′]bipyrazinyl-5′-carboxylic acid methyl ester was prepared from isopropylpiperazine and 5-Chloro-pyrazine-2-carboxylic acid methyl ester following general procedure H.

m/z: (M+H)⁺ 265

### b) 3-Isopropyl-4-(quinolin-5-ylthiocarbamoyl)-3,4,5,6-tetrahydro-2H-[1,2′]bipyrazinyl-5′-carboxylic acid methyl ester

The title compound was prepared from 3-Isopropyl-3,4,5,6-tetrahydro-2H-[1,2′]bipyrazinyl-5′-carboxylic acid methyl ester and 5-Isothiocyanato-quinoline following general procedure N.

m/z: (M+H)⁺ 451

### c) 3-Isopropyl-4-(quinolin-5-ylthiocarbamoyl)-3,4,5,6-tetrahydro-2H-[1,2′]bipyrazinyl-5′-carboxylic acid

To a solution of 3-Isopropyl-4-(quinolin-5-ylthiocarbamoyl)-3,4,5,6-tetrahydro-2H-[1,2′]bipyrazinyl-5′-carboxylic acid methyl ester (2 g, 4.4 mmol) in dioxane (20 mL) was added lithium hydroxide (565 mg, 13.5 mmol) in water (10 mL) at ambient temperature. The reaction was allowed to stir for 6 hours upon which time it was concentrated to give 360 mg (0.825 mmol) of the title compound.

m/z: (M+H)⁺ 435.

### d) 5-(3-isopropyl-4-(quinolin-5-ylcarbamothioyl)piperazin-1-yl)-N-aminopyrazine-2-carboxamide, 5-(3-isopropyl-4-(quinolin-5-ylcarbamothioyl)piperazin-1-yl)-N,N-dimethylpyrazine-2-carboxamide, and N-(2-aminoethyl)-5-(3-isopropyl-4-(quinolin-5-ylcarbamothioyl)piperazin-1-yl)pyrazine-2-carboxamide

The title compounds were prepared from 3-Isopropyl-4-(quinolin-5-ylthiocarbamoyl)-3,4,5,6-tetrahydro-2H-[1,2′]bipyrazinyl-5′-carboxylic acid and methylamine, dimethylamine, or ethylene diamine, respectively, following general procedure D.
5-(3-isopropyl-4-(quinolin-5-ylcarbamothioyl)piperazin-1-yl)-N-methylpyrazine-2-carboxamide

Retention time: 1.56 min (method d):

m/z: (M+H)⁺ 450.
5-(3-isopropyl-4-(quinolin-5-ylcarbamothioyl)piperazin-1-yl)-N,N-dimethylpyrazine-2-carboxamide

Retention time: 1.50 min (method d)

m/z: (M+H)⁺ 464
N-(2-aminoethyl)-5-(3-isopropyl-4-(quinolin-5-ylcarbamothioyl)piperazin-1-yl)pyrazine-2-carboxamide

Retention time: 1.31 min (method d):

m/z: (M+H)⁺ 478

### Examples 53 and 54

### Preparation of N′-cyano-2-isopropyl-4-(7-methyl-7H-pyrrolo[2,3-d]pyrimidin-4-yl)-N-(quinolin-5-yl)piperazine-1-carboximidamide and 2-isopropyl-4-(7-methyl-7H-pyrrolo[2,3-d]pyrimidin-4-yl)-N-(quinolin-5-yl)piperazine-1-carbothioamide

### a) 4-(3-Isopropyl-piperazin-1-yl)-7-methyl-7H-pyrrolo[2,3-d]pyrimidine

The title compound was prepared from isopropyl piperazine and 4-Chloro-7-methyl-7H-pyrrolo[2,3-d]pyrimidine following general procedure H.

m/z: (M+H)⁺ 260

### b) N′-cyano-2-isopropyl-4-(7H-pyrrolo[2,3-d]pyrimidin-4-yl)-N-(quinolin-5-yl)piperazine-1-carboximidamide and 2-Isopropyl-4-(7H-pyrrolo[2,3-d]pyrimidin-4-yl)-piperazine-1-carbothioic acid quinolin-5-ylamide

The title compounds were prepared from 4-(3-Isopropyl-piperazin-1-yl)-7-methyl-7H-pyrrolo[2,3-d]pyrimidine and 1-Isocyanato-naphthalene following general procedure G. This material was employed in the following step without further purification.

### c) N′-cyano-2-isopropyl-4-(7-methyl-7H-pyrrolo[2,3-d]pyrimidin-4-yl)-N-(quinolin-5-yl)piperazine-1-carboximidamide and 2-isopropyl-4-(7-methyl-7H-pyrrolo[2,3-d]pyrimidin-4-yl)-N-(quinolin-5-yl)piperazine-1-carbothioamide

To a solution of A,-cyano-2-isopropyl-4-(7H-pyrrolo[2,3-d]pyrimidin-4-yl)-N-(quinolin-5-yl)piperazine-1-carboximidamide and 2-Isopropyl-4-(7H-pyrrolo[2,3-d]pyrimidin-4-yl)-piperazine-1-carbothioic acid quinolin-5-ylamide (360 mg) in DMF (100 mL) was added sodium hydride (75 mg, 3.3 mmol) followed by methyl iodide (0.210 mL, 3.3 mmol) and the reaction was allowed to stir for 16 hours. The three products were purified by HPLC.
N′-cyano-2-isopropyl-4-(7-methyl-7H-pyrrolo[2,3-d]pyrimidin-4-yl)-N-(quinolin-5-yl)piperazine-1-carboximidamide

Retention time: 1.54 min (method d)

m/z: (M+H)⁺ 452.
2-isopropyl-4-(7-methyl-7H-pyrrolo[2,3-d]pyrimidin-4-yl)-N-(quinolin-5-yl)piperazine-1-carbothioamide

Retention time: 2.32 min (method d)

m/z: (M+H)⁺ 444.

### Examples 55 and 56

### Preparation of 4-(2-chlorothieno[3,2-d]pyrimidin-4-yl)-N′-cyano-2-isopropyl-N-(1,2,3,4-tetrahydroisoquinolin-5-yl)piperazine-1-carboximidamide and 4-(2-chlorothieno[3,2-d]pyrimidin-4-yl)-2-isopropyl-N-(1,2,3,4-tetrahydroisoquinolin-5-yl)piperazine-1-carbothioamide

### a) 2-Chloro-4-(3-isopropyl-piperazin-1-yl)-thieno[2,3-d]pyrimidine

The title compound was prepared from isopropyl piperazine and 2,4-Dichloro-thieno[2,3-d]pyrimidine following general procedure H.

m/z: (M+H)⁺ 297

### b) (9H-fluoren-9-yl)methyl 5-(4-(2-chlorothieno[2,3-d]pyrimidin-4-yl)-N′-cyano-2-isopropylpiperazine-1-carboximidamido)-3,4-dihydroisoquinoline-2(1H)-carboxylate and 5-{[4-(2-Chloro-thieno[2,3-d]pyrimidin-4-yl)-2-isopropyl-piperazine-1-carbothioyl]-amino}-3,4-dihydro-1H-isoquinoline-2-carboxylic acid 9H-fluoren-9-ylmethyl ester

The title compounds were prepared from 2-Chloro-4-(3-isopropyl-piperazin-1-yl)-thieno[2,3-d]pyrimidine and 5-Isothiocyanato-3,4-dihydro-1H-isoquinoline-2-carboxylic acid 9H-fluoren-9-ylmethyl ester following general procedure G.

(9H-fluoren-9-yl)methyl 5-(4-(2-chlorothieno[2,3-d]pyrimidin-4-yl)-NA-cyano-2-isopropylpiperazine-1-carboximidamido)-3,4-dihydroisoquinoline-2(1H)-carboxylate

m/z: (M+H)⁺ 718
5-{[4-(2-Chloro-thieno[2,3-d]pyrimidin-4-yl)-2-isopropylpiperazine-1-carbothioyl]-amino}-3,4-dihydro-1H-isoquinoline-2-carboxylic acid 9H-fluoren-9-ylmethyl ester

m/z: (M+H)⁺ 710

### c) 4-(2-chlorothieno[3,2-d]pyrimidin-4-yl)-2-isopropyl-N-(1,2,3,4-tetrahydroisoquinolin-5-yl)piperazine-1-carbothioamide and 4-(2-chlorothieno[3,2-d]pyrimidin-4-yl)-N′-cyano-2-isopropyl-N-(1,2,3,4-tetrahydroisoquinolin-5-yl)piperazine-1-carboximidamide

The title compounds were prepared from (9H-fluoren-9-yl)methyl 5-(4-(2-chlorothieno[2,3-d]pyrimidin-4-yl)-N′-cyano-2-isopropylpiperazine-1-carboximidamido)-3,4-dihydroisoquinoline-2(1H)-carboxylate and 5-{[4-(2-Chloro-thieno[2,3-d]pyrimidin-4-yl)-2-isopropylpiperazine-1-carbothioyl]-amino}-3,4-dihydro-1H-isoquinoline-2-carboxylic acid 9H-fluoren-9-ylmethyl ester following general procedure P.
4-(2-chlorothieno[3,2-d]pyrimidin-4-yl)-2-isopropyl-N-(1,2,3,4-tetrahydroisoquinolin-5-yl)piperazine-1-carbothioamide

Retention time; 1.49 min (method d)

m/z: (M+H)⁺ 487
4-(2-chlorothieno[3,2-d]pyrimidin-4-yl)-N′-cyano-2-isopropyl-N-(1,2,3,4-tetrahydroisoquinolin-5-yl)piperazine-1-carboximidamide

Retention time; 1.46 min (method d)

m/z: (M+H)⁺ 495

### Examples 57 and 58

### Preparation of 4-(imidazo[1,2-b]pyridazin-6-yl)-2-isopropyl-N-(quinolin-5-yl)piperazine-1-carbothioamide acetate and N′-cyano-4-(imidazo[1,2-b]pyridazin-6-yl)-2-isopropyl-N-(quinolin-5-yl)piperazine-1-carboximidamide

### a) 6-Chloro-imidazo[1,2-b]pyridazine

To a solution of 3-chloro-6-aminopyridazine (18.7 g, 144 mmol) in DMF (300 mL) was added chloroacetal (46 mL, 361 mmol) at ambient temperature. The reaction was allowed to stir at ambient temperature for 16 hours upon which the reaction was concentrated and the resulting solid was dissolved in dichloromethane. This solution was washed with water, dried over magnesium sulfate and concentrated to yield 10.0 g (65 mmol) of a green solid.

m/z: (M+H)⁺ 154

### b) 6-(3-Isopropyl-piperazin-1-yl)-imidazo[1,2-b]pyridazine

The title compound was prepared from 6-Chloro-imidazo[1,2-b]pyridazine and isopropylpiperazine following general procedure H.

m/z: (M+H)⁺ 246

### c) 4-(imidazo[1,2-b]pyridazin-6-yl)-2-isopropyl-N-(quinolin-5-yl)piperazine-1-carbothioamide acetate and N′-cyano-4-(imidazo[1,2-b]pyridazin-6-yl)-2-isopropyl-N-(quinolin-5-yl)piperazine-1-carboximidamide

The title compounds were prepared from 6-(3-Isopropyl-piperazin-1-yl)-imidazo[1,2-b]pyridazine and 1-Isocyanato-naphthalene following general procedure G.
4-(imidazo[1,2-b]pyridazin-6-yl)-2-isopropyl-N-(quinolin-5-yl)piperazine-1-carbothioamide acetate

Retention time: 2.08 min (method d)

m/z: (M+H)⁺ 431
N′-cyano-4-(imidazo[1,2-b]pyridazin-6-yl)-2-isopropyl-N-(quinolin-5-yl)piperazine-1-carboximidamide

Retention time: 2.01 min (method d)

m/z: (M+H)⁺ 439.

### Example 59

### Preparation of 5-(4-(N′-cyano-N-o-tolylcarbamimidoyl)-3-isopropylpiperazin-1-yl)-N,N-dimethylpyrazine-2-carboxamide

### a) 3-Isopropyl-3,4,5,6-tetrahydro-2H-[1,2′]bipyrazinyl-5′-carboxylic acid methyl ester

3-Isopropyl-3,4,5,6-tetrahydro-2H-[1,2′]bipyrazinyl-5′-carboxylic acid methyl ester was prepared from isopropylpiperazine and 5-Chloro-pyrazine-2-carboxylic acid methyl ester following general procedure H.

m/z: (M+H)⁺ 265

### b) 3-Isopropyl-2,3,5,6-tetrahydro-[1,2′]bipyrazinyl-4,5′-dicarboxylic acid 4-benzyl ester 5′-methyl ester

The title compound was prepared from Carbonic acid benzyl ester 2,5-dioxo-pyrrolidin-1-yl ester and 3-Isopropyl-3,4,5,6-tetrahydro-2H-[1,2′]bipyrazinyl-5′-carboxylic acid methyl ester following general procedure L.

m/z: (M+H)⁺ 399

### c) 3-Isopropyl-2,3,5,6-tetrahydro-[1,2′]bipyrazinyl-4,5′-dicarboxylic acid 4-benzyl ester

To a solution of 3-Isopropyl-2,3,5,6-tetrahydro-[1,2′]bipyrazinyl-4,5′-dicarboxylic acid 4-benzyl ester (6.6 g, 25 mmol) in dioxane (250 mL) was added lithium hydroxine (3.2 g, 75 mmol) in water (50 mL) at ambient temperature. The reaction was allowed to stir at ambient temperature for 4 hours upon which time it was concentrated and acetic acid was added until a white precipitate formed. The white precipitate was filtered to yield the title compound (9.4 g, 24.5 mmol) as a white solid

m/z: (M+H)⁺ 385

### d) 5′-Dimethylcarbamoyl-3-isopropyl-2,3,5,6-tetrahydro-[1,2′]bipyrazinyl-4-carboxylic acid benzyl ester

The title compound was prepared from 3-Isopropyl-2,3,5,6-tetrahydro-[1,2′]bipyrazinyl-4,5′-dicarboxylic acid 4-benzyl ester and dimethyl amine following general procedure D.

m/z: (M+H)⁺ 412

### e) 3-Isopropyl-3,4,5,6-tetrahydro-2H-[1,2′]bipyrazinyl-5′-carboxylic acid dimethylamide

The title compound was prepared following general procedure M.

m/z: (M+H)⁺ 278

### f) 5-(4-(N′-cyano-N-o-tolylcarbanimidoyl)-3-isopropylpiperazin-1-yl)-N,N-dimethylpyrazine-2-carboxamide

The title compound was prepared from 3-Isopropyl-3,4,5,6-tetrahydro-2H-[1,2′]bipyrazinyl-5′-carboxylic acid dimethylamide and 1-Isothiocyanato-2-methyl-benzene following general procedure G.
5-(4-(N′-cyano-N-o-tolylcarbamimidoyl)-3-isopropylpiperazin-1-yl)-N,N-dimethylpyrazine-2-carboxamide

Retention time: 2.33 min (method a)

m/z: (M+H)⁺ 435

### Example 60

### Preparation of 5-(4-(N′-cyano-N-o-tolylcarbamimidoyl)-2-phenylpiperazin-1-yl)-N,N-dimethylpyrazine-2-carboxamide

### a) 3-Phenyl-piperazine-1-carboxylic acid benzyl ester

The title compound was prepared from 2-phenylpiperazine and Carbonic acid benzyl ester 2,5-dioxo-pyrrolidin-1-yl ester following general procedure L.

m/z: (M+H)⁺ 297

### b) 2-Phenyl-2,3,5,6-tetrahydro-[1,2′]bipyrazinyl-4,5′-dicarboxylic acid 4-benzyl ester 5′-methyl ester

The title compound was prepared from 3-Phenyl-piperazine-1-carboxylic acid benzyl ester and 5-Chloro-pyrazine-2-carboxylic acid methyl ester following general procedure H.

m/z: (M+H)⁺ 433

### c) 5′-Dimethylcarbamoyl-2-phenyl-2,3,5,6-tetrahydro-[1,2′]bipyrazinyl-4-carboxylic acid benzyl ester

To a solution of 2-Phenyl-2,3,5,6-tetrahydro-[1,2′]bipyrazinyl-4,5′-dicarboxylic acid 4-benzyl ester 5′-methyl ester (950 mg, 2.2 mmol) in dioxane (10 mL) was added lithium hydroxide (370 mg, 8.8 mmol) in water (2 mL) at ambient temperature. The reaction was heated to 85° C. and the reaction was allowed to stir for 30 minutes. The reaction was cooled and concentrated followed by addition of acetic acid until a precipitate was formed. The precipitate was filtered washed with ether and dissolved in dichloromethane (25 mL). Carbonyldiimidizole was added (1.66 g, 10 mmol) followed by dimethylaminopyridine (250 mg, 2 mmol) and dimethylamine (5 mL, 2.5 mmol) and the reaction was allowed to stir at ambient temperature for 2 hours. 50 mL of 10% an aqueous citric acid solution was added and the organic layer was separated, dried over magnesium sulfate and concentrated to give 5′-Dimethylcarbamoyl-2-phenyl-2,3,5,6-tetrahydro-[1,2′]bipyrazinyl-4-carboxylic acid benzyl ester (1 g, 2.6 mmol) as a white solid.

m/z: (M+H)⁺ 385

### d) 2-Phenyl-3,4,5,6-tetrahydro-2H-[1,2′]bipyrazinyl-5′-carboxylic acid dimethylamide

The title compound was prepared from 5′-Dimethylcarbamoyl-2-phenyl-2,3,5,6-tetrahydro-[1,2′]bipyrazinyl-4-carboxylic acid benzyl ester following general procedure M.

m/z: (M+H)⁺ 312

### e) 5-(4-(N′-cyano-N-o-tolylcarbamimidoyl)-2-phenylpiperazin-1-yl)-N,N-dimethylpyrazine-2-carboxamide

The title compound was prepared from 2-Phenyl-3,4,5,6-tetrahydro-2H-[1,2′]bipyrazinyl-5′-carboxylic acid dimethylamide and 1-Isothiocyanato-2-methyl-benzene following general procedure G.
5-(4-(N′-cyano-N-o-tolylcarbamimidoyl)-2-phenylpiperazin-1-yl)-N,N-dimethylpyrazine-2-carboxamide

Retention time: 2.35 min (method a)

m/z: (M+H)⁺ 469.

### Example 61

### Preparation of 5-(4-(N′-cyano-N-(2-methylquinolin-5-yl)carbamimidoyl)-3-isopropylpiperazin-1-yl)-N,N-dimethylpyrazine-2-carboxamide

### a) 3-Isopropyl-3,4,5,6-tetrahydro-2H-[1,2′]bipyrazinyl-5′-carboxylic acid methyl ester

3-Isopropyl-3,4,5,6-tetrahydro-2H-[1,2′]bipyrazinyl-5′-carboxylic acid methyl ester was prepared from 2-isopropylpiperazine and 5-Chloro-pyrazine-2-carboxylic acid methyl ester following general procedure H.

m/z: (M+H)⁺ 265

### b) 3-Isopropyl-2,3,5,6-tetrahydro-[1,2′]bipyrazinyl-4,5′-dicarboxylic acid 4-benzyl ester 5′-methyl ester

The title compound was prepared from Carbonic acid benzyl ester 2,5-dioxo-pyrrolidin-1-yl ester and 3-Isopropyl-3,4,5,6-tetrahydro-2H-[1,2′]bipyrazinyl-5′-carboxylic acid methyl ester following general procedure L.

m/z: (M+H)⁺ 399

### c) 3-Isopropyl-2,3,5,6-tetrahydro-[1,2′]bipyrazinyl-4,5′-dicarboxylic acid 4-benzyl ester

To a solution of 3-Isopropyl-2,3,5,6-tetrahydro-[1,2′]bipyrazinyl-4,5′-dicarboxylic acid 4-benzyl ester (6.6 g, 25 mmol) in dioxane (250 mL) was added lithium hydroxide (3.2 g, 75 mmol) in water (50 mL) at ambient temperature. The reaction was allowed to stir at ambient temperature for 4 hours upon which time it was concentrated and acetic acid was added until a white precipitate formed. The white precipitate was filtered to yield the title compound (9.4 g, 24.5 mmol) as a white solid.

m/z: (M+H)⁺ 385

### d) 5′-Dimethylcarbamoyl-3-isopropyl-2,3,5,6-tetrahydro-[1,2′]bipyrazinyl-4-carboxylic acid benzyl ester

The title compound was prepared from 3-Isopropyl-2,3,5,6-tetrahydro-[1,2′]bipyrazinyl-4,5′-dicarboxylic acid 4-benzyl ester and dimethyl amine following general procedure D.

m/z: (M+H)⁺ 412

### e) 3-Isopropyl-3,4,5,6-tetrahydro-2H-[1,2′]bipyrazinyl-5′-carboxylic acid dimethylamide

The title compound was prepared from 5′-Dimethylcarbamoyl-3-isopropyl-2,3,5,6-tetrahydro-[1,2′]bipyrazinyl-4-carboxylic acid benzyl ester following general procedure M.

m/z: (M+H)⁺ 278

### f) 5-(4-(N′-cyano-N-(2-methylquinolin-5-yl)carbamimidoyl)-3-isopropylpiperazin-1-yl)-N,N-dimethylpyrazine-2-carboxamide

The title compound was prepared from 3-Isopropyl-3,4,5,6-tetrahydro-2H-[1,2′]bipyrazinyl-5′-carboxylic acid dimethylamide and 5-Isothiocyanato-2-methylquinoline following general procedure G.
5-(4-(N′-cyano-N-(2-methylquinolin-5-yl)carbamimidoyl)-3-isopropylpiperazin-1-yl)-N,N-dimethylpyrazine-2-carboxamide

Retention time: 2.09 min (method a)

m/z: (M+H)⁺ 486.

### Example 62

### Preparation of 2-isopropyl-4-(2-morpholinothieno[3,2-d]pyrimidin-4-yl)-N-(quinolin-5-yl)piperazine-1-carbothioamide

### a) 2-Chloro-4-(3-isopropyl-piperazin-1-yl)-thieno[3,2-d]pyrimidine

The title compound was prepared from 2,4-Dichloro-thieno[3,2-d]pyrimidine and isopropylpiperazine following general procedure H.

m/z: (M+H)⁺ 297

### b) 4-(3-Isopropyl-piperazin-1-yl)-2-morpholin-4-yl-thieno[3,2-d]pyrimidine

The title compound was prepared from morpholine and 2-Chloro-4-(3-isopropyl-piperazin-1-yl)-thieno[3,2-d]pyrimidine following general procedure H.

m/z: (M+H)⁺ 348

### c) 2-isopropyl-4-(2-morpholinothieno[3,2-d]pyrimidin-4-yl)-N-(quinolin-5-yl)piperazine-1-carbothioamide

The title compound was prepared from 4-(3-Isopropyl-piperazin-1-yl)-2-morpholin-4-yl-thieno[3,2-d]pyridine and 5-Isothiocyanatoquinoline following general procedure G.
2-isopropyl-4-(2-morpholinothieno[3,2-d]pyrimidin-4-yl)-N-(quinolin-5-yl)piperazine-1-carbothioamide

Retention time: 1.77 min (method d)

m/z: (M+H)⁺ 534

### Example 63

### Preparation of 4-(2-(1,3-dihydroxypropan-2-ylamino)thieno[3,2-d]pyrimidin-4-yl)-N-(1H-indol-4-yl)-2-isopropylpiperazine-1-carbothioamide

### a) 2-Chloro-4-(3-isopropyl-piperazin-1-yl)-thieno[3,2-d]pyrimidine

The title compound was prepared from 2,4-Dichloro-thieno[3,2-d]pyrimidine and isopropylpiperazine following general procedure H.

m/z: (M+H)⁺ 297

### b) {Hydroxymethyl-[4-(3-isopropyl-piperazin-1-yl)-thieno[3,2-d]pyrimidin-2-yl]-amino}-methanol

The title compound was prepared from morpholine and 2-Chloro-4-(3-isopropyl-piperazin-1-yl)-thieno[3,2-d]pyrimidine following general procedure H.

m/z: (M+H)⁺ 338

### c) 4-(2-(1,3-dihydroxypropan-2-ylamino)thieno[3,2-d]pyrimidin-4-yl)-N-(1H-indol-4-yl)-2-isopropylpiperazine-1-carbothioamide

The title compound was prepared from {Hydroxymethyl-[4-(3-isopropyl-piperazin-1-yl)-thieno[3,2-d]pyrimidin-2-yl]-amino}-methanol and 4-Isothiocyanato-1H-indole following general procedure G.
4-(2-(1,3-dihydroxypropan-2-ylamino)thieno[3,2-d]pyrimidin-4-yl)-N-(1H-indol-4-yl)-2-isopropylpiperazine-1-carbothioamide

Retention time: 1.43 min (method d)

m/z: (M+H)⁺ 526

### Example 64

### Preparation of N′-cyano-4-(2-(dimethylamino)thieno[3,2-d]pyrimidin-4-yl)-2-isopropyl-N-(quinolin-5-yl)piperazine-1-carboximidamide

### a) 2-Chloro-4-(3-isopropyl-piperazin-1-yl)-thieno[3,2-d]pyrimidine

The title compound was prepared from 2,4-Dichloro-thieno[3,2-d]pyrimidine and isopropylpiperazine following general procedure H.

m/z: (M+H)⁺ 297

### b) [4-(3-Isopropyl-piperazin-1-yl)-thieno[2,3-d]pyrimidin-2-yl]-dimethyl-amine

The title compound was prepared from 2-Chloro-4-(3-isopropyl-piperazin-1-yl)-thieno[3,2-d]pyrimidine and dimethylamine following general procedure H.

m/z: (M+H)⁺ 306

### c) N′-cyano-4-(2-(dimethylamino)thieno[3,2-d]pyrimidin-4-yl)-2-isopropyl-N-(quinolin-5-yl)piperazine-1-carboximidamide

The title compound was prepared from [4-(3-Isopropyl-piperazin-1-yl)-thieno[2,3-d]pyrimidin-2-yl]-dimethyl-amine and 5-Isothiocyanato-quinoline following general procedure G.
N′-cyano-4-(2-(dimethylamino)thieno[3,2-d]pyrimidin-4-yl)-2-isopropyl-N-(quinolin-5-yl)piperazine-1-carboximidamide

Retention time: 2.53 (method a)

m/z: (M+H)⁺ 500

### Example 65

### Preparation of 4-(2-chloropyrimidin-4-yl)-3,3-dimethyl-N-(quinolin-5-yl)piperazine-1-carbothioamide acetate

### a) 3,3-Dimethyl-piperazine-1-carbothioic acid quinolin-5-ylamide

The title compound was prepared from 2,2-dimethylpiperazine and 5-Isothiocyanato-quinoline following general procedure N.

m/z: (M+H)⁺ 301

### b) 4-(2-chloropyrimidin-4-yl)-3,3-dimethyl-N-(quinolin-5-yl)piperazine-1-carbothioamide acetate

The title compound was prepared from 3,3-Dimethyl-piperazine-1-carbothioic acid quinolin-5-ylamide and 2,4-dichloropyrimidine following general procedure H.
4-(2-chloropyrimidin-4-yl)-3,3-dimethyl-N-(quinolin-5-yl)piperazine-1-carbothioamide acetate

Retention time: 1.70 min (method d)

m/z: (M+H)⁺ 412

### Example 66

### Preparation of Tetrahydro-pyran-4-carboxylic acid {6-[3-isopropyl-4-(quinolin-5-ylthiocarbamoyl)-piperazin-1-yl]-pyridazin-3-yl}-amide

### a) 3-Chloro-6-(3-isopropyl-piperazin-1-yl)-pyridazine

The title compound was prepared from isopropylpiperazine and 3,6-dichloropyridazine following general procedure H.

m/z: (M+H)⁺ 241

### b) [6-(3-Isopropyl-piperazin-1-yl)-pyridazin-3-yl]-(4-methoxy-benzyl)-anine

The title compound was prepared from 3-Chloro-6-(3-isopropyl-piperazin-1-yl)-pyridazine and 4-methoxybenzylamine following general procedure H.

m/z: (M+H)⁺ 342

### c) 6-(3-Isopropyl-piperazin-1-yl)-pyridazin-3-ylamine

[6-(3-Isopropyl-piperazin-1-yl)-pyridazin-3-yl]-(4-methoxy-benzyl)-amine (8.7 g, 25.5 mmol) in trifluoroacetic acid (40 mL) was heated to 60° C. for 2 hours upon which the reaction was concentrated and used without purification.

### d) 4-(6-Amino-pyridazin-3-yl)-2-isopropyl-piperazine-1-carbothioic acid quinolin-5-ylamide

The title compound was prepared from 6-(3-Isopropyl-piperazin-1-yl)-pyridazin-3-ylamine and 5-Isothiocyanato-quinoline following general procedure N.

m/z: (M+H)⁺ 408

### e) Tetrahydro-pyran-4-carboxylic acid {6-[3-isopropyl-4-(quinolin-5-ylthiocarbamoyl)-piperazin-1-yl]-pyridazin-3-yl}-amide

The title compound was prepared from 4-(6-Amino-pyridazin-3-yl)-2-isopropyl-piperazine-1-carbothioic acid quinolin-5-ylamide and Tetrahydro-pyran-4-carboxylic acid following general procedure D.
Tetrahydro-pyran-4-carboxylic acid {6-[3-isopropyl-4-(quinolin-5-ylthiocarbamoyl)-piperazin-1-yl]-pyridazin-3-yl}-amide

Retention time: 1.56 min (method d)

m/z: (M+H)⁺ 520

### Example 67

### Preparation of N-{6-[3-Isopropyl-4-(quinolin-5-ylthiocarbamoyl)-piperazin-1-yl]-pyridazin-3-yl}-acetamide

### a) 3-Chloro-6-(3-isopropyl-piperazin-1-yl)-pyridazine

The title compound was prepared from isopropylpiperazine and 3,6-dichloropyridazine following general procedure H.

m/z: (M+H)⁺ 241

### b) [6-(3-Isopropyl-piperazin-1-yl)-pyridazin-3-yl]-(4-methoxy-benzyl)-amine

The title compound was prepared from 3-Chloro-6-(3-isopropyl-piperazin-1-yl)-pyridazine and 4-methoxybenzylamine following general procedure H.

m/z: (M+H)⁺ 342

### c) 6-(3-Isopropyl-piperazin-1-yl)-pyridazin-3-ylamine

[6-(3-Isopropyl-piperazin-1-yl)-pyridazin-3-yl]-(4-methoxy-benzyl)-amine (8.7 g, 25.5 mmol) in trifluoroacetic acid (40 mL) was heated to 60° C. for 2 hours upon which the reaction was concentrated and used without purification.

### d) 4-(6-Amino-pyridazin-3-yl)-2-isopropyl-piperazine-1-carbothioic acid quinolin-5-ylamide

The title compound was prepared from 6-(3-Isopropyl-piperazin-1-yl)-pyridazin-3-ylamine and 5-Isothiocyanato-quinoline following general procedure N.

m/z: (M+H)⁺ 408

### e) N-{6-[3-Isopropyl-4-(quinolin-5-ylthiocarbamoyl)-piperazin-1-yl]-pyridazin-3-yl}-acetamide

The title compound was prepared from 4-(6-Amino-pyridazin-3-yl)-2-isopropyl-piperazine-1-carbothioic acid quinolin-5-ylamide and acetic acid following general procedure D.
N-{6-[3-Isopropyl-4-(quinolin-5-ylthiocarbamoyl)-piperazin-1-yl]-pyridazin-3-yl}-acetamide

Retention time: 1.64 min (method d)

m/z: (M+H)⁺ 492

### Example 68

### Preparation of 3-Hydroxy-N-{6-[3-isopropyl-4-(quinolin-5-ylthiocarbamoyl)-piperazin-1-yl]-pyridazin-3-yl}-propionamide

### a) 3-Chloro-6-(3-isopropyl-piperazin-1-yl)-pyridazine

The title compound was prepared from isopropylpiperazine and 3,6-dichloropyridazine following general procedure H.

m/z: (M+H)⁺ 241

### b) [6-(3-Isopropyl-piperazin-1-yl)-pyridazin-3-yl]-(4-methoxy-benzyl)amine

The title compound was prepared from 3-Chloro-6-(3-isopropyl-piperazin-1-yl)-pyridazine and 4-methoxybenzylamine following general procedure H.

m/z: (M+H)⁺ 342

### c) 6-(3-Isopropyl-piperazin-1-yl)-pyridazin-3-ylamine

[6-(3-Isopropyl-piperazin-1-yl)-pyridazin-3-yl]-(4-methoxy-benzyl)-amine (8.7 g, 25.5 mmol) in trifluoroacetic acid (40 mL) was heated to 60° C. for 2 hours upon which the reaction was concentrated and used without purification.

### d) 4-(6-Amino-pyridazin-3-yl)-2-isopropyl-piperazine-1-carbothioic acid quinolin-5-ylamide

The title compound was prepared from 6-(3-Isopropyl-piperazin-1-yl)-pyridazin-3-ylamine and 5-Isothiocyanato-quinoline following general procedure N.

m/z: (M+H)⁺ 408

### e) 3-Hydroxy-N-{6-[3-isopropyl-4-(quinolin-5-ylthiocarbamoyl)-piperazin-1-yl]-pyridazin-3-yl}-propionamide

The title compound was prepared from 4-(6-Amino-pyridazin-3-yl)-2-isopropyl-piperazine-1-carbothioic acid quinolin-5-ylamide and 3-Hydroxy-propionic acid following general procedure D.
3-Hydroxy-N-{6-[3-isopropyl-4-(quinolin-5-ylthiocarbamoyl)-piperazin-1-yl]-pyridazin-3-yl}-propionamide

Retention time: 1.46 min (method d)

m/z: (M+H)⁺ 480

### Examples 69 and 70

### Preparation of N′-cyano-2-isopropyl-4-(3-(morpholine-4-carbonyl)pyrazin-2-yl)-N-(quinolin-5-yl)piperazine-1-carboximidamide and 2-isopropyl-4-(3-(morpholine-4-carbonyl)pyrazin-2-yl)-N-(quinolin-5-yl)piperazine-1-carbothioamide acetate

### a) 3′-Chloro-3-isopropyl-3,4,5,6-tetrahydro-2H-[1,2′]bipyrazinyl

The title compound was prepared from 2,6-dichloropyrazine and isopropylpiperazine following general procedure H.

m/z: (M+H)⁺ 241

### b) (3-Isopropyl-3,4,5,6-tetrahydro-2H-[1,2′]bipyrazinyl-3′-yl)-morpholin-4-yl-methanone

The title compound was prepared from 3′-Chloro-3-isopropyl-3,4,5,6-tetrahydro-2H-[1,2′]bipyrazinyl and carbon monoxide following general procedure Q.

m/z: (M+H)⁺ 320

### c) N′-cyano-2-isopropyl-4-(3-(morpholine-4-carbonyl)pyrazin-2-yl)-N-(quinolin-5-yl)piperazine-1-carboximidamide and 2-isopropyl-4-(3-(morpholine-4-carbonyl)pyrazin-2-yl)-N-(quinolin-5-yl)piperazine-1-carbothioamide acetate

The title compounds were prepared from (3-Isopropyl-3,4,5,6-tetrahydro-2H-[1,2′]bipyrazinyl-3′-yl)-morpholin-4-yl-methanone which was prepared from 3′-Chloro-3-isopropyl-3,4,5,6-tetrahydro-2H-[1,2′]bipyrazinyl, carbon monoxide and 5-Isothiocyanatoquinoline following general procedure G.
N′-cyano-2-isopropyl-4-(3-(morpholine-4-carbonyl)pyrazin-2-yl)-N-(quinolin-5-yl)piperazine-1-carboximidamide

m/z: (M+H)⁺ 514

Retention time: 1.5 min (method d)
2-isopropyl-4-(3-(morpholine-4-carbonyl)pyrazin-2-yl)-N-(quinolin-5-yl)piperazine-1-carbothioamide acetate

m/z: (M+H)⁺ 507

Retention time: 1.59 min (method d)

### Example 71

### Preparation of N′-cyano-2-isopropyl-4-(3-ethylpyrazin-2-yl)-N-(quinolin-5-yl)piperazine-1-carboximidamide

### a) 3′-Chloro-3-isopropyl-3,4,5,6-tetrahydro-2H-[1,2′]bipyrazinyl

The title compound was prepared from 2,6-dichloropyrazine and 2-isopropylpiperazine following general procedure H.

m/z: (M+H)⁺ 241

### b) 3-Ethyl-6-(3-isopropyl-piperazin-1-yl)-pyridazine

To a solution of 3′-Chloro-3-isopropyl-3,4,5,6-tetrahydro-2H-[1,2′]bipyrazinyl (700 mg, 2.9 mmol) in THF and NMP (10 mL and 1.5 mL respectively) was added iron acetylacetone (52 mg, 0.15 mmol) and ethylmagnesium bromide 3.63 mL, 7.25 mmol) dropwise over 15 minutes at ambient temperature. After 1 hr the reaction was concentrated and purified by silica gel chromatography (9/1 ethylacetate/methanol) to give 870 mg 2.7 mmol) as a clear light red oil.

m/z: (M+H)⁺ 235

### c) N′-cyano-2-isopropyl-4-(3-(ethyl)pyrazin-2-yl)-N-(quinolin-5-yl)piperazine-1-carboximidamide

The title compound was prepared from 3-Ethyl-6-(3-isopropyl-piperazin-1-yl)-pyridazine and 5-Isothiocyanato-quinoline following general procedure G.
N′-cyano-2-isopropyl-4-(3-ethylpyrazin-2-yl)-N-(quinolin-5-yl)piperazine-1-carboximidamide

m/z: (M+H)⁺ 429

Retention time: 1.73 min (method d)

### Example 72

### Preparation of benzyl 5-(N′-cyano-2-isopropyl-4-(4-oxo-3,4-dihydroquinazolin-2-yl)piperazine-1-carboximidamido)-3,4-dihydroisoquinoline-2(1H)-carboxylate

benzyl 5-(N′-cyano-2-isopropyl-4-(4-oxo-3,4-dihydroquinazolin-2-yl)piperazine-1-carboximidamido)-3,4-dihydroisoquinoline-2(1H)-carboxylate was prepared from 5-Isothiocyanato-3,4-dihydro-1H-isoquinoline-2-carboxylic acid benzyl ester and 2-(3-Isopropylpiperazin-1-yl)-3H-quinazolin-4-one using general procedure G.

Retention time: 1.90 min. (method d)

m/z: (M+H)⁺ 605.

### Example 73

### Preparation of Benzyl 5-(2-isopropyl-4-(4-oxo-3,4-dihydroquinazolin-2-yl)piperazine-1-carbothioamido)-3,4-dihydroisoquinoline-2(1H)-carboxylate

Benzyl 5-(2-isopropyl-4-(4-oxo-3,4-dihydroquinazolin-2-yl)piperazine-1-carbothioamido)-3,4-dihydroisoquinoline-2(1H)-carboxylate was prepared from 5-Isothiocyanato-3,4-dihydro-1H-isoquinoline-2-carboxylic acid benzyl ester and 2-(3-Isopropylpiperazin-1-yl)-3H-quinazolin-4-one using general procedure G.

Retention time: 2.05 min. (method d)

m/z: (M+H)⁺ 597.

### Preparation 2: Preparation of N-(4-piperazin-2-ylphenyl)acetamide

A mixture of N-[4-(2-oxoacetyl)phenyl]acetamide hydrate (3.00 g, 14.3 mmol) in absolute ethanol (25 mL) was cooled to 2° C. and ethylenediamine (0.91 g, 15.2 mmol) was added drop-wise over a five minute time period, keeping the temperature less than 10° C. The mixture was allowed to rise to ambient temperature and stirred for 18 hours. The solution was cooled to 2° C. and sodium borohydride (0.87 g, 22.9 mmol) was added in portions over a ten minute time period. The reaction mixture was allowed to come to ambient temperature and stirred for 3 days. The solvent was removed in vacuo and the residue was partitioned between water (30 mL) and chloroform (20 mL). The aqueous phase was subjected to continuous liquid/liquid extraction with chloroform for 8 hours. The chloroform was removed in vacuo to give N-(4-piperazin-2-ylphenyl)acetamide as a yellow solid (2.127 g, 9.7 mmol); ¹H NMR (DMSO-d₆, 400 MHz) (rotomers) δ 9.87-9.96 (m, 1H), 7.46-7.58 (m, 2H), 7.29 (d, 1.5H, J=8.5 Hz), 7.25 (d, 0.5H, J=8.4 Hz), 3.63-3.76 (m, 1H), 2.53-3.05 (m, 5H), 2.02 (s, 3H), 0.92-1.06 (m, 1H).

### Preparation 3: Preparation of 2-(4-Methanesulfonylphenyl)piperazine

A solution of ethylenediamine (1.70 g, 28.3 mmol) in absolute ethanol (20 mL) was cooled to 10° C. and 2-Bromo-1-(4-methanesulfonylphenyl)ethanone (1.02 g, 3.7 mmol) was added over a ten minute time period, keeping the temperature less than 10° C. The mixture was allowed to rise to ambient temperature and stirred for 3 days. The solvent was removed in vacuo and the residue was dissolved in absolute ethanol (20 mL). The solution was cooled to 2° C. and sodium borohydride (0.27 g, 7.0 mmol) was added in portions over a ten minute time period. The reaction mixture was allowed to come to ambient temperature and stirred for 18 hours. The solvent was removed in vacuo and the residue was partitioned between water (5 mL) and chloroform (10 mL). The layers were separated and the aqueous phase was extracted with chloroform (3×10 mL). The combined chloroform phases were dried over anhydrous magnesium sulfate and the solvent was removed in vacuo. The residue was purified by column chromatography on silica using methanol/dichloromethane/aqueous ammonia (30:69.5:0.5) as an eluent to give 2-(4-methanesulfonylphenyl)piperazine as a yellow solid (0.131 g, 0.5 mmol); ¹H NMR (DMSO-d₆, 400 MHz) δ 7.89 (d, 2H, J=8.3 Hz), 7.60 (d, 2H, J=8.4 Hz), 3.80-3.91 (m, 1H), 3.05 (s, 3H), 2.82-3.14 (m, 5H), 2.61-2.70 (m, 1H).

### Preparation 4: Preparation of 2-Propylpiperazine

To a solution of 2-propyl-pyrazine (1.0 g, 8.18 mmol) in ethanol (35 mL) was added palladium on carbon (10%, 0.1 g). The reaction mixture was shaken under hydrogen (50 psi) for 2 days. The catalyst was filtered off and the solvent was removed under vacuum to yield the product (0.945 g, 7.37 mmol). ¹H NMR (DMSO-d₆, 400 MHz) δ 2.68 (m, 3H), 2.34-2.55 (m, 3H), 2.06 (m, 1H), 1.23 (m, 2H), 1.12 (m, 2H), 0.83 (m, 3H).

### Preparation 5: Preparation of 2,2-dipropylpiperazine

### a) 2-Bromo-2-propyl-pentanal

To the solution of 2-propyl-pentanal (5.10 g, 39.78 mmol) in ether (20 mL) was added dioxane (0.2 mL) followed by two drops of bromine. The reaction flask was immersed in a cold water bath and bromine was added dropwise (2.0 mL, 39.38). After 5 minutes, the reaction mixture was poured onto ice. After stirring for 2 minutes, sodium carbonate (2.20 g, 20.8 mmol) was added in small portions. The layers were separated and the aqueous was extracted with ether. The combined organic layer was washed with brine, dried over magnesium sulfate, filtered and concentrated in vacuum to yield 2-bromo-2-propyl-pentanal (8.47 g, 100%) which was used as is. ¹H NMR (CDCl₃) δ 7.51 (s, 1H), 3.47 (m, 2H), 2.88 (m, 2H), 1.40 (m, 8H), 0.93 (m, 6H).

### b) 6,6-Dipropyl-1,2,3,6-tetrahydro-pyrazine

The crude 2-bromo-2-propyl-pentanal from a) (39.78 mmol) was added to a solution of ethane-1,2-diamine (26 mL, 397.8 mmol) in toluene (24 mL). After 1 hour, the reaction mixture was heated at 115° C. for half an hour. After cooling to room temperature, the layers were separated and the solvent in the toluene layer was removed to yield the product (8.07 g 45.77 mmol) which was used in the subsequent step without further purification. ¹H NMR (CDCl₃) δ 4.51 (s, 1H), 3.47 (m, 2H), 2.88 (m, 2H), 1.40 (m, 8H), 0.93 (m, 6H).

### c) 2,2-Dipropyl-piperazine

To the crude product from b) in ethanol (35 mL) was added palladium on carbon (10%, 0.8 g). The reaction mixture was shaken under hydrogen (32 psi) for 4 days. The catalyst was filtered off and the solvent was removed under vacuum to yield the title compound (6.09 g, 35.76 mmol) which was used without any further purification. 1H NMR (CDCl₃) δ 2.81 (m, 4H), 2.63 (s, 2H), 1.71 (bs, 2H), 1.19-1.52 (m, 8H), 1.93 (m, 6H).

### Preparation 6: Preparation of 2-butylpiperazine

### a) 2-Bromohexanal

To a solution of hexanal (5.02 g, 50.1 mmol) in ether (18 mL) and dioxane (0.2 mL), bromine (8.0 g, 50.1 mmol) was added dropwise at 0° C. Upon completion of the addition, the ice-bath was removed and the reaction mixture was stirred at ambient temperature for 1 hour; it was poured into ice-cold water (50 mL) and the organic phase was separated. It was dried with magnesium sulfate and concentrated to yield 2-bromohexanal (8.1 g, 45.3 mmol) as a yellow oil.

m/z: (M+H)⁺ 179,181 (method m).

### b) 6-Butyl-1,2,3,6-tetrahydropyrazine

To the solution of ethylenediamine (20.7 g, 344.0 mmol) in toluene (18 mL), 2-bromohexanal (6.16 g, 34.4 mmol) was added dropwise and the resulting mixture was stirred at ambient temperature for 2 hours. The reaction mixture was diluted with water (250 mL); the organic layer was separated, dried with magnesium sulfate and concentrated to yield 6-butyl-1,2,3,6-tetrahydropyrazine (4.34 g, 31.0 mmol) as a yellow oil.

m/z: (M+H)⁺ 141 (method m).

### c) 2-Butylpiperazine

To a solution of 6-butyl-1,2,3,6-tetrahydropyrazine (1.5 g, 10.7 mmol) in EtOH (250 mL) was added 10% palladium on carbon (2.2 g). The mixture was shaken under hydrogen (60 psi) for 72 hours. The catalyst was removed by filtration and the solvent was removed under reduced pressure. The residue was suspended in ether (100 mL) and the solid was filtered. The filtrate was concentrated under reduced pressure to yield 2-butylpiperazine (0.8 g, 5.63 mmol) as a yellow solid.

m/z: (M+H)⁺ 143 (method m).

### Example 74

### Preparation of N′-cyano-2-isopropyl-4-(4-oxo-3,4-dihydroquinazolin-2-yl)-N-(1,2,3,4-tetrahydroisoquinolin-5-yl)piperazine-1-carboximidamide acetate

N′-cyano-2-isopropyl-4-(4-oxo-3,4-dihydroquinazolin-2-yl)-N-(1,2,3,4-tetrahydroisoquinolin-5-yl)piperazine-1-carboximidamide acetate was prepared from benzyl 5-(N′-cyano-2-isopropyl-4-(4-oxo-3,4-dihydroquinazolin-2-yl)piperazine-1-carboximidamido)-3,4-dihydroisoquinoline-2(1H)-carboxylate using general procedure M.

Retention time: 0.76 min. (method d)

m/z: (M+H)⁺ 471.

### Example 75

### Preparation of 1-(4-chlorophenylcarbamoyl)-4-(N′-cyano-N-o-tolylcarbamimidoyl)piperazine-2-carboxylic acid

### a) piperazine-2-carboxylic acid ethyl ester

To a solution of 1,4-dibenzyl-piperazine-2-carboxylic acid ethyl ester (7.227 g, 21.35 mmol) in ethanol (200 mL) was added palladium on carbon (10% Pd, 0.725 g). The reaction mixture was hydrogenated under hydrogen (50 psi) for 3 days. The catalyst was filtered off and the solvent was removed under vacuum to yield the titled product (3.37 g, 21.30 mmol). ¹H NMR (DMSO, d₆) δ 4.07 (q, 2H), 3.28 (m, 1H), 2.91 (m, 1H), 2.79 (m, 1H), 2.60 (m, 2H), 2.53 (m, 2H), 2.26 (bs, 2H), 1.18 (t, 3H).

### b) ethyl 4-(N′-cyano-N-o-tolylcarbamimidoyl)piperazine-2-carboxylate

To a solution of piperazine-2-carboxylic acid ethyl ester (1.96 g, 12.39 mmol) was added phenyl N′-cyano-N-o-tolylcarbamimidate (3.11 g, 12.39 mmol). The mixture was stirred at 80° C. for 18 hours. The solvent was removed and the residue was purified by flash silica gel column chromatography, eluting with ethyl acetate/methanol (85/15) to yield the titled compound (2.66 g, 8.43 mmol). ¹H NMR (DMSO, d₆) δ 8.9 (s, 1H), 7.17 (m, 3H), 7.12 (m, 1H), 4.12 (m, 2H), 3.86 (m, 1H), 3.58 (m, 1H), 3.46 (m, 1H), 3.31 (m, 1H), 3.25 (m, 1H), 2.89 (m, 1H), 2.81 (bs, 1H), 2.62 (m, 1H), 2.19 (m, 3H), 1.20 (t, 3H).

### c) 2-(4-chlorophenyl)-N′-cyano-1,3-dioxo-N-o-tolylhexahydroimidazo[1,5-a]pyrazine-7(1H)-carboximidamide and ethyl 4-(N′-cyano-N-o-tolylcarbamimidoyl)-1-(phenylcarbamoyl)piperazine-2-carboxylate

To a solution of the product from above (1.0 g, 3.17 mmol) in acetonitrile (10 mL) at 0° C. was added a solution of 1-chloro-4-isocyanato-benzene (0.487 g, 3.17 mmol) in acetonitrile (5 mL) slowly. After 3 hours, the solvent was removed and the residue was purified by flash silica gel chromatography, eluting with dichloromethane/ethyl acetate (70/30) to give the titled compounds as a mixture (1.18 g).
2-(4-chlorophenyl)-N′-cyano-1,3-dioxo-N-o-tolylhexahydroimidazo[1,5-a]pyrazine-7(11H)-carboximidamide

Retention time: 2.38 min. (method g)

m/z: (M+H)⁺ 423.
ethyl 4-(N′-cyano-N-o-tolylcarbamimidoyl)-1-(phenylcarbamoyl)piperazine-2-carboxylate

Retention time: 2.38 min. (method g)

m/z: (M+H)⁺ 469.

### d) 1-(4-chlorophenylcarbamoyl)-4-(N′-cyano-N-o-tolylcarbamimidoyl)piperazine-2-carboxylic acid

To a suspension of the mixture from c) (0.5 g) in ethanol (3 mL) was added sodium hydroxide (1.0 N, 2.1 mL). After 5 minutes, the organic solvent was removed and the aqueous solution neutralized with HCl (2.0 N, 1.1 mL). The solid was collected by filtration to give the titled product (0.47 g, 1.06 mmol).

Retention time: 0.82 min. (method g)

m/z: (M+H)⁺ 441.

### Example 76

### Preparation of N-(4-chlorophenyl)-4-(N′-cyano-N-o-tolylcarbamimidoyl)-2-(hydroxymethyl)piperazine-1-carboxamide

To a suspension of the mixture of 2-(4-chlorophenyl)-N′-cyano-1,3-dioxo-N-o-tolylhexahydroimidazo[1,5-a]pyrazine-7(1H)-carboximidamide and ethyl 4-(N′-cyano-N-o-tolylcarbamimidoyl)-1-(phenylcarbamoyl)piperazine-2-carboxylate (1.06 g) in ethanol (3 mL) was added lithium borohydride (0.49 g, 22.6 mmol) in 3 portions. HCl (1.0 N, 25 mL) was added to quench the reaction after 2 hours. 5 minutes after the complete addition of HCL, a saturated solution of sodium bicarbonate was added to neutralize the reaction mixture. The organic solvent was removed and the aqueous layer was extracted with dichloromethane. The combined organic layers were washed with brine, dried over magnesium sulfate, filtered and concentrated. The residue was purified by flash silica gel chromatography using dichloromethane/ethyl acetate (50/50) to yield the titled product (0.051 g 0.12 mmol).

Retention time: 2.25 min. (method g)

m/z: (M+H)⁺ 427.

### Example 77

### Preparation of 4-(N-(2-(aminomethyl)phenyl)-N′-cyanocarbamimidoyl)-N-(4-chlorophenyl)-2-phenylpiperazine-1-carboxamide

### a) (2-Amino-benzyl)-carbamic acid benzyl ester

To a solution of 2-aminomethyl-phenylamine (0.82 g, 6.75 mmol) in dichloromethane (67 mL) was added triethylamine (1.13 mL, 8.10 mmol). The reaction mixture was cooled to 0° C., benzyl chloroformate (0.96 mL, 6.75 mmol) was added dropwise. After stirring at room temperature for 18 hours, the organic layer was washed with water, brine, dried over magnesium sulfate, filtered and concentrated. The residue was purified by flash silica gel chromatography using dichloromethane/ethyl acetate (80/20) to yield the titled product (1.34 g, 5.23 mmol).

Retention time: 2.70 min. (method g)

m/z: (M+H)⁺ 257.

### b) benzyl 2-((cyanoimino)(phenoxy)methylamino)benzylcarbamate

benzyl 2-((cyanoimino)(phenoxy)methylamino)benzylcarbamate was prepared from (2-amino-benzyl)-carbamic acid benzyl ester and diphenyl cyanocarbonimidate using general procedure A.

m/z: (M+H)⁺ 401.

### c) benzyl 2-(N′-cyano-3-phenylpiperazine-1-carboximidamido)benzylcarbamate

benzyl 2-(N′-cyano-3-phenylpiperazine-1-carboximidamido)benzylcarbamate was prepared from benzyl 2-((cyanoimino)(phenoxy)methylamino)benzylcarbamate and 2-phenylpiperazine using general procedure B.

m/z: (M+H)⁺ 469.

d) benzyl 2-(4-(4-chlorophenylcarbamoyl)-N′-cyano-3-phenylpiperazine-1-carboximidamido)benzylcarbamate

benzyl 2-(4-(4-chlorophenylcarbamoyl)-N′-cyano-3-phenylpiperazine-1-carboximidamido)benzylcarbamate was prepared from benzyl 2-(N′-cyano-3-phenylpiperazine-1-carboximidamido)benzylcarbamate and 1-chloro-4-isocyanato-benzene using general procedure C.

m/z: (M+H)⁺ 622.

### e) 4-(N-(2-(aminomethyl)phenyl)-N′-cyanocarbamimidoyl)-N-(4-chlorophenyl)-2-phenylpiperazine-1-carboxamide

4-(N-(2-(aminomethyl)phenyl)-N′-cyanocarbamimidoyl)-N-(4-chlorophenyl)-2-phenylpiperazine-1-carboxamide was prepared from benzyl 2-(4-(4-chlorophenylcarbamoyl)-N′-cyano-3-phenylpiperazine-1-carboximidamido)benzylcarbamate using general procedure M.

1H NMR (DMSO, d₆) δ 8.74 (s, 1H), 7.51 (m, 2H), 7.36 (m, 6H), 7.22 (m, 1H), 7.00 (m, 1H), 6.87 (m, 1H), 6.73 (m, 2H), 5.47 (m, 1H), 4.57 (m, 1H), 4.28 (m, 2H), 4.05 (m, 1H), 3.93 (m, 1H), 3.43 (m, 1H), 3.21 (m, 1H), 3.07 (m, 1H).

m/z: (M+H)⁺ 488.

### Example 78

### Preparation of 4-benzyl-N′-cyano-3-(methoxymethyl)-N-o-tolylpiperazine-1-carboximidamide

### a) (1,4-Dibenzylpiperazin-2-yl)-methanol

To a suspension of lithium aluminum hydride (3.14 g, 82.74 mmol) in tetrahydrofuran (50 mL) was added a solution of 1,4-dibenzyl-piperazine-2-carboxylic acid ethyl ester (10 g, 29.55 mmol) in tetrahydrofuran (100 mL) over 1 hour. 30 minutes after the complete addition, water (3.2 mL) was added followed by sodium hydroxide (2.0 N, 9.2 mL). The precipitate was filtered and the solvent was removed. The residue was purified by flash silica gel chromatography using dichloromethane/methanol (90/10) to yield the titled product (7.86 g, 26.55 mmol).

m/z: (M+H)⁺ 297.

### b) 1,4-Dibenzyl-2-methoxymethyl-piperazine

To a suspension of sodium hydride (0.4 g, 10.11 mmol) in ether (5 mL) was added a solution of (1,4-Dibenzyl-piperazin-2-yl)-methanol (1.0 g, 3.37 mmol) in N,N-dimethylformamide (3 mL). 45 minutes after the complete addition, a solution of methyl iodide (0.28 mL, 4.48 mmol) in ether (2 mL) was added dropwise. The reaction mixture was stirred for 18 hours before water was added dropwise and the layers were separated. The aqueous layer was extracted with ether (×4) and the combined organic layer was washed with water, brine, dried over magnesium sulfate, filtered and concentrated to yield the titled product (0.556 g, 1.80 mmol).

m/z: (M+H)⁺ 311.

### c) 1-Benzyl-2-methoxymethyl-piperazine

To a solution of 1,4-Dibenzyl-2-methoxymethyl-piperazine (0.56 g, 1.78 mmol) in ethanol (20 mL) was added palladium on carbon (10%, 50 mg). The reaction mixture was shaken under hydrogen (50 psi) for 18 hours. The catalyst was filtered off and the solvent was removed under vacuum to yield a mixture of 1-benzyl-2-methoxymethyl-piperazine and 2-methoxymethyl-piperazine (0.26 g) which was used in the subsequent step without further purification.

### d) 4-benzyl-N′-cyano-3-(methoxymethyl)-N-o-tolylpiperazine-1-carboximidamide

4-benzyl-N′-cyano-3-(methoxymethyl)-N-o-tolylpiperazine-1-carboximidamide was prepared from the mixture from step c (1-benzyl-2-methoxymethyl-piperazine and 2-methoxymethyl-piperazine) and phenyl N′-cyano-N-o-tolylcarbamimidate using general procedure B.

Retention time: 2.56 min. (method g)

m/z: (M+H)⁺ 378.

### Example 79

### Preparation of N-(4-chlorophenyl)-4-(N′-cyano-N-o-tolylcarbamimidoyl)-2-((2-methoxy)methyl)piperazine-1-carboxamide

N-(4-chlorophenyl)-4-(N′-cyano-N-o-tolylcarbamimidoyl)-2-((2-methoxy)methyl)piperazine-1-carboxamide was prepared from N′-cyano-3-(methoxymethyl)-N-o-tolylpiperazine-1-carboximidamide and 1-chloro-4-isocyanato-benzene using general procedure C.

Retention time: 2.67 min. (method g)

m/z: (M+H)⁺ 441.

### Example 80

### Preparation of N-(4-chlorophenyl)-4-(N′-cyano-N-o-tolylcarbamimidoyl)-2-((2-methoxyethoxy)methyl)piperazine-1-carboxamide

### a) 1,4-Dibenzyl-2-(2-methoxy-ethoxymethyl)-piperazine

To a suspension of sodium hydride (0.81 g, 20.24 mmol) in ether (10 mL) was added a solution of (1,4-dibenzyl-piperazin-2-yl)-methanol (2.0 g, 6.75 mmol) in N,N-dimethylformamide (6 mL). After 45 minutes, a solution of 1-chloro-2-methoxy-ethane (0.92 mL, 10.13 mmol) in ether (4 mL) was added dropwise. The reaction mixture was stirred for 18 hours before more 1-chloro-2-methoxy-ethane (0.92 mL, 10.13 mmol) was added. After 4 hours, water was added dropwise and the layers were separated. The aqueous layer was extracted with ether (×4) and the combined organic layers were washed with water, brine, dried over magnesium sulfate, filtered and concentrated. The residue was purified by flash silica gel chromatography using ethyl acetate to yield the titled product (1.01 g, 2.85 mmol).

Retention time: 3.50 min. (method g)

m/z: (M+H)⁺ 355.

### b) 2-(2-Methoxy-ethoxymethyl)-piperazine

To a solution of 1,4-dibenzyl-2-(2-methoxy-ethoxymethyl)-piperazine (1.0 g, 2.82 mmol) in ethanol (40 mL) was added palladium on carbon (10%, 200 mg). The reaction mixture was shaken under hydrogen (50 psi) for 3 days. The catalyst was filtered off and the solvent was removed under vacuum to yield the titled compound (0.47 g, 2.70 mmol).

### c) N′-cyano-3-((2-methoxyethoxy)methyl)-N-o-tolylpiperazine-1-carboximidamide

N′-cyano-3-((2-methoxyethoxy)methyl)-N-o-tolylpiperazine-1-carboximidamide was prepared from 2-(2-Methoxy-ethoxymethyl)-piperazine and phenyl N′-cyano-N-o-tolylcarbamimidate using general procedure B.

m/z: (M+H)⁺ 288.

### d) N-(4-chlorophenyl)-4-(N′-cyano-N-o-tolylcarbamimidoyl)-2-((2-methoxyethoxy)methyl)piperazine-1-carboxamide

N-(4-chlorophenyl)-4-(N′-cyano-N-o-tolylcarbamimidoyl)-2-((2-methoxyethoxy)methyl)piperazine-1-carboxamide was prepared from N′-cyano-3-((2-methoxyethoxy)methyl)-N-o-tolylpiperazine-1-carboximidamide and 1-chloro-4-isocyanatobenzene using general procedure C.

Retention time: 1.95 min. (method d)

m/z: (M+H)⁺ 485.

### Example 81

### Preparation of N-(4-chlorophenyl)-4-(N′-cyano-N-o-tolylcarbamimidoyl)-2-(((2-methoxyethoxy)methoxy)methyl)piperazine-1-carboxamide

### a) 1,4-Dibenzyl-2-(2-methoxy-ethoxymethoxymethyl)-piperazine

To a solution of (1,4-dibenzyl-piperaziN-2-yl)methanol (2.0 g, 6.75 mmol) and diisopropylethyl amine (1.4 mL, 8.20 mmo) in dichloromethane (10 mL) was added 1-chloromethoxy-2-methoxyethane (0.89 mL, 7.78 mmol). The reaction mixture was stirred for 18 hours. A saturated solution of sodium bicarbonate was added to the reaction and the layers were separated. The aqueous layer was extracted with dichloromathane and the combined organic layers were washed with water, brine, dried over magnesium sulfate, filtered and concentrated. The residue was purified by flash silica gel chromatography using ethyl acetate to yield the titled product (0.714 g, 1.86 mmol).

Retention time: 3.59 min. (method g)

m/z: (M+H)⁺ 385.

### b) 2-(2-Methoxy-ethoxymethoxymethyl)-piperazine

To a solution of 1,4-dibenzyl-2-(2-methoxy-ethoxymethoxymethyl)-piperazine (0.714 g, 1.86 mmol) in ethanol (30 mL) was added palladium on carbon (10%, 295 mg). The reaction mixture was shaken under hydrogen (50 psi) for 3 days. The catalyst was filtered off and the solvent was removed under vacuum to yield the titled compound (0.35 g, 1.71 mmol).

m/z: (M+H)⁺ 205.

### c) N′-cyano-3-(((2-methoxyethoxy)methoxy)methyl)-N-o-tolylpiperazine-1-carboximidamide

N′-cyano-3-(((2-methoxyethoxy)methoxy)methyl)-N-o-tolylpiperazine-1-carboximidamide was prepared from 2-(2-Methoxy-ethoxymethoxymethyl)-piperazine and phenyl N′-cyano-N-o-tolylcarbamimidate using general procedure B.

m/z: (M+H)⁺ 288.

### d) N-(4-chlorophenyl)-4-(N′-cyano-N-o-tolylcarbamimidoyl)-2-(((2-methoxyethoxy)methoxy)methyl)piperazine-1-carboxamide

N-(4-chlorophenyl)-4-(N′-cyano-N-o-tolylcarbamimidoyl)-2-(((2-methoxyethoxy)methoxy)methyl)piperazine-1-carboxamide was prepared from N′-cyano-3-(((2-methoxyethoxy)methoxy)methyl)-N-o-tolylpiperazine-1-carboximidamide and 1-chloro-4-isocyanato-benzene using general procedure C.

Retention time: 1.95 min. (method d)

m/z: (M+H)⁺ 485.

### Example 82

### Preparation of N′-cyano-2,2-dimethyl-4-(1-phenyl-1H-tetrazol-5-yl)-N-o-tolylpiperazine-1-carboximidamide

A mixture of N′-cyano-2,2-dimethyl-N-o-tolylpiperazine-1-carboximidamide (50 mg, 0.184 mmol), 5-methyl-1-phenyl-1H-tetrazole (33 mg, 0.184 mmol), 18 crown 6 (73 mg, 0.276 mmol), potassium fluoride (53 mg, 0.92 mmol), and triethyl amine (0.052 mL, 0.368 mmol) in dioxane (0.5 mL) was stirred at room temperature for 18 hours. The solvent is removed in vacuo and the product was purified by reverse-phase HPLC to give the titled product (43 mg, 0.103 mmol).

Retention time: 1.90 min. (method d)

m/z: (M+H)⁺ 416.

### Example 83

### Preparation of benzyl 5-(N′-cyano-4-(5-(dimethylcarbamoyl)pyrazin-2-yl)-2-isopropylpiperazine-1-carboximidamido)-3,4-dihydroisoquinoline-2(1H)-carboxylate

### a) 3-Isopropyl-3,4,5,6-tetrahydro-2H-[1,2′]bipyrazinyl-5′-carboxylic acid methyl ester

3-Isopropyl-3,4,5,6-tetrahydro-2H-[1,2′]bipyrazinyl-5′-carboxylic acid methyl ester was prepared from 2-isopropylpiperazine and 5-chloro-pyrazine-2-carboxylic acid methyl ester using general procedure H.

Retention time: 1.10 min. (method d)

m/z: (M+H)⁺ 265.

### b) benzyl 5-(N′-cyano-2-isopropyl-4-(5-(methoxycarbonyl)pyrazin-2-yl)piperazine-1-carboximidamido)-3,4-dihydroisoquinoline-2(1H)-carboxylate

benzyl 5-(N′-cyano-2-isopropyl-4-(5-(methoxycarbonyl)pyrazin-2-yl)piperazine-1-carboximidamido)-3,4-dihydroisoquinoline-2(1H)-carboxylate was prepared from 3-isopropyl-3,4,5,6-tetrahydro-2H-[1,2′]bipyrazinyl-5′-carboxylic acid methyl ester and 5-isothiocyanato-3,4-dihydro-1H-isoquinoline-2-carboxylic acid benzyl ester using general procedure G.

Retention time: 1.89 min. (method d)

m/z: (M+H)⁺ 597.

### c) Lithium 5-(4-(N-(2-(benzyloxycarbonyl)-1,2,3,4-tetrahydroisoquinolin-5-yl)-N′-cyanocarbamimidoyl)-3-isopropylpiperazin-1-yl)pyrazine-2-carboxylate

To a solution of benzyl 5-(N′-cyano-2-isopropyl-4-(5-(methoxycarbonyl)pyrazin-2-yl)piperazine-1-carboximidamido)-3,4-dihydroisoquinoline-2(1H)-carboxylate (3.06 g, 5.13 mmol) in dioxane (25 mL) was added a solution of lithium hydroxide in water (6 mL). The reaction mixture was stirred at room temperature for 5 hours. The organic solvent was removed and the aqueous was lyophilized to yield the titled compound (2.97 g, 5.05 mmol).

Retention time: 1.56 min. (method d)

m/z: (M+H)⁺ 583.

### d) benzyl 5-(N′-cyano-4-(5-(dimethylcarbamoyl)pyrazin-2-yl)-2-isopropylpiperazine-1-carboximidamido)-3,4-dihydroisoquinoline-2(1H)-carboxylate

To a solution of lithium 5-(4-(N-(2-(benzyloxycarbonyl)-1,2,3,4-tetrahydroisoquinolin-5-yl)-N′-cyanocarbamimidoyl)-3-isopropylpiperazin-1-yl)pyrazine-2-carboxylate (2.97 mmol, 5.04 mmol) in N,N-dimethylformamide (25 mL) was added N,N-diisopropylethylamine (4.4 mL, 25.2 mmol) and N,N-dimethyl amine (2.0N in THF, 10 mL, 20.16 mmol). A solution of O-7-azobenzotriazol-1-yl)-N,N,N′N′-tetramethyluronium hexafluorophosphate (3.83 g, 10.08 mmol) in N,N-dimethylformamide (5 mL) was then added slowly. After stirring at room temperature for 1 hour, the reaction mixture was poured onto ice. The aqueous layer was extracted with dichloromathane and the combined organic layer was washed with water, brine, dried over magnesium sulfate, filtered and concentrated. The residue was purified by flash silica gel chromatography using ethyl acetate/methanol (90/10) to yield the titled product (3.0 g, 4.92 mmol).

Retention time: 1.75 min. (method d)

m/z: (M+H)⁺ 610.

### e) 5-(4-(N′-cyano-N-(1,2,3,4-tetrahydroisoquinolin-5-yl)carbamimidoyl)-3-isopropylpiperazin-1-yl)-N,N-dimethylpyrazine-2-carboxamide

5-(4-(N′-cyano-N-(1,2,3,4-tetrahydroisoquinolin-5-yl)carbamimidoyl)-3-isopropylpiperazin-1-yl)-N,N-dimethylpyrazine-2-carboxamide was prepared from benzyl 5-(N′-cyano-4-(5-(dimethylcarbamoyl)pyrazin-2-yl)-2-isopropylpiperazine-1-carboximidamido)-3,4-dihydroisoquinolin-2(1H)-carboxylate using general procedure M.

Retention time: 1.25 min. (method d)

m/z: (M+H)⁺ 476.

### Example 84

### Preparation of 5-(4-(N′-cyano-N-(2-propyl-1,2,3,4-tetrahydroisoquinolin-5-yl)carbamimidoyl)-3-isopropylpiperazin-1-yl)-N,N-dimethylpyrazine-2-carboxamide

5-(4-(N′-cyano-N-(2-propyl-1,2,3,4-tetrahydroisoquinolin-5-yl)carbamimidoyl)-3-isopropylpiperazin-1-yl)-N,N-dimethylpyrazine-2-carboxamide was prepared from 5-(4-(N′-cyano-N-(1,2,3,4-tetrahydroisoquinolin-5-yl)carbamimidoyl)-3-isopropylpiperazin-1-yl)-N,N-dimethylpyrazine-2-carboxamide and N-propanal using general procedure J.

Retention time: 1.31 min. (method d)

m/z: (M+H)⁺ 518.

### Example 85

### Preparation of 5-(4-(N′-cyano-N-(2-(cyclopropylmethyl)-1,2,3,4-tetrahydroisoquinolin-5-yl)carbamimidoyl)-3-isopropylpiperazin-1-yl)-N,N-dimethylpyrazine-2-carboxamide

5-(4-(N′-cyano-N-(2-(cyclopropylmethyl)-1,2,3,4-tetrahydroisoquinolin-5-yl)carbamimidoyl)-3-isopropylpiperazin-1-yl)-N,N-dimethylpyrazine-2-carboxamide was prepared from 5-(4-(N′-cyano-N-(1,2,3,4-tetrahydroisoquinolin-5-yl)carbamimidoyl)-3-isopropylpiperazin-1-yl)-N,N-dimethylpyrazine-2-carboxamide and cyclopropanecarbaldehyde using general procedure J.

Retention time: 1.33 min. (method d)

m/z: (M+H)⁺ 530.

### Example 86

### Preparation of 5-(4-(N′-cyano-N-(2-isopropyl-1,2,3,4-tetrahydroisoquinolin-5-yl)carbamimidoyl)-3-isopropylpiperazin-1-yl)-N,N-dimethylpyrazine-2-carboxamide

5-(4-(N′-cyano-N-(2-isopropyl-1,2,3,4-tetrahydroisoquinolin-5-yl)carbamimidoyl)-3-isopropylpiperazin-1-yl)-N,N-dimethylpyrazine-2-carboxamide was prepared from 5-(4-(N′-cyano-N-(1,2,3,4-tetrahydroisoquinolin-5-yl)carbamimidoyl)-3-isopropylpiperazin-1-yl)-N,N-dimethylpyrazine-2-carboxamide and acetone using general procedure J.

Retention time: 1.30 min. (method d)

m/z: (M+H)⁺ 518.

### Example 87

### Preparation of 6-(4-(N′-cyano-N-(2-methylquinolin-5-yl)carbamimidoyl)-3-isopropylpiperazin-1-yl)-N,N-dimethylpyridazine-3-carboxamide

### a) 3-Bromo-6-(3-isopropyl-piperazin-1-yl)-pyridazine

3-Bromo-6-(3-isopropyl-piperazin-1-yl)-pyridazine was prepared from 2-isopropylpiperazine and 3,6-dibromo-pyridazine using general procedure H.

Retention time: 1.17 min. (method d)

m/z: (M+H)⁺ 285.

### b) 6-(3-Isopropyl-piperazin-1-yl)-pyridazine-3-carboxylic acid dimethylamide

6-(3-Isopropyl-piperazin-1-yl)-pyridazine-3-carboxylic acid dimethylamide was prepared from 3-bromo-6-(3-isopropyl-piperazin-1-yl)pyridazine using general procedure Q.

Retention time: 0.92 min. (method d)

m/z: (M+H)⁺ 278.

### c) 6-(4-(N′-cyano-N-(2-methylquinolin-5-yl)carbamimidoyl)-3-isopropylpiperazin-1-yl)-N,N-dimethylpyridazine-3-carboxamide

6-(4-(N′-cyano-N-(2-methylquinolin-5-yl)carbamimidoyl)-3-isopropylpiperazin-1-yl)-N,N-dimethylpyridazine-3-carboxamide was prepared from reaction 6-(3-isopropyl-piperazin-1-yl)-pyridazine-3-carboxylic acid dimethylamide with 5-isothiocyanato-2-methylquinoline using general procedure G.

Retention time: 1.41 min. (method d)

m/z: (M+H)⁺ 486.

### Example 88

### Preparation of (S)—N′-Cyano-4-(2-(3,4-dimethoxyphenyl)acetyl)-3-isopropyl-N-o-tolylpiperazine-1-carboximidamide

### a) (S)-2-(3,4-Dimethoxyphenyl)-1-(3-isopropylpiperazin-1-yl)ethanone

(S)-2-(3,4-Dimethoxyphenyl)-1-(3-isopropylpiperazin-1-yl)ethanone was prepared from (3,4-dimethoxyphenyl)acetylchloride and (S)-2-isopropylpiperazine using general procedure D.

Retention time: 0.74 min. (method i)

m/z: (M+H)⁺ 307.

### b) (S)—N′-Cyano-4-(2-(3,4-dimethoxyphenyl)acetyl)-3-isopropyl-N-o-tolylpiperazine-1-carboximidamide

(S)—N′-Cyano-4-(2-(3,4-dimethoxyphenyl)acetyl)-3-isopropyl-N-o-tolylpiperazine-1-carboximidamide was prepared from (S)-2-(3,4-dimethoxyphenyl)-1-(3-isopropylpiperazin-1-yl)ethanone and phenyl N′-cyano-N-o-tolylcarbamimidate using general procedure B.

Retention time: 2.15 min. (method i)

m/z: (M+H)⁺ 464.

### Example 89

### Preparation of (S)—N′-Cyano-4-(2-(3,4-dimethoxyphenyl)acetyl)-3-isobutyl-N-o-tolylpiperazine-1-carboximidamide

### a) (S)-2-(3,4-Dimethoxyphenyl)-1-(3-isobutylpiperazin-1-yl)ethanone

(S)-2-(3,4-Dimethoxyphenyl)-1-(3-isobutylpiperazin-1-yl)ethanone was prepared from (3,4-dimethoxyphenyl)acetylchloride and (S)-2-isobutylpiperazine using general procedure D.

Retention time: 0.80 min. (method i)

m/z: (M+H)⁺ 321.

### b) (S)—N′-Cyano-4-(2-(3,4-dimethoxyphenyl)acetyl)-3-isobutyl-N-o-tolylpiperazine-1-carboximidamide

(S)—N′-Cyano-4-(2-(3,4-dimethoxyphenyl)acetyl)-3-isobutyl-N-o-tolylpiperazine-1-carboximidamide was prepared from (S)-2-(3,4-dimethoxyphenyl)-1-(3-isobutylpiperazin-1-yl)ethanone and phenyl N′-cyano-N-o-tolylcarbamimidate using general procedure B.

Retention time: 2.30 min. (method i)

m/z: (M+H)⁺ 478.

### Example 90

### Preparation of 3-(4-(2-(3,4-Dimethoxyphenyl)acetyl)-2-isopropylpiperazin-1-yl)-4-(o-tolylamino)cyclobut-3-ene-1,2-dione

At 0° C., the o-toluidine (0.227 mL, 2.111 mmol) was added dropwise to a solution of 3,4-dimethoxy-3-cyclobutene-1,2-dione (300 mg, 2.111 mmol) and triethylamine (0.30 mL, 2.111 mmol) in methanol (5 mL). The reaction was allowed to stir at 0° C. for 2 hours before the dropwise addition of 2-(3,4-dimethoxyphenyl)-1-(3-isopropylpiperazin-1-yl)ethanone (647 mg, 2.111 mmol) in methanol (5 mL). After the addition was complete, the reaction was allowed to warm to ambient temperature. The product was purified by reverse-phase HPLC on a Hyperprep HS C18 column, 8 gm, 250×21.2 mm; 20% acetonitrile-50 mM ammonium acetate over 1 min, 20-60% acetonitrile-50 mM ammonium acetate for 34 min, 60-100% acetonitrile for 1 min, 100% acetonitrile for 5 min, 25 mL/min. The product was isolated by lyophilization of the desired fractions to give 3-(4-(2-(3,4-dimethoxyphenyl)acetyl)-2-isopropylpiperazin-1-yl)-4-(o-tolylamino)cyclobut-3-ene-1,2-dione (106 mg, 0.215 mmol).

Retention time: 2.20 min. (method i)

m/z: (M+H)⁺ 492.

### Example 91

### Preparation of N-(4-Chlorophenyl)-4-(3,4-dioxo-2-(o-tolylamino)cyclobut-1-enyl)-2-phenylpiperazine-1-carboxamide

### a) N-(4-Chlorophenyl)-2-phenylpiperazine-1-carboxamide

At 0° C., a solution of 4-chlorophenylisocyanate (4.99 g, 32.48 mmol) in tetrahydrofuran (20 ml) was added dropwise to a solution of 3-phenylpiperazine-1-carboxylic acid tert-butyl ester (8.52 g, 32.48 mmol) in tetrahydrofuran (30 mL). The reaction was allowed to warm to ambient temperature before the solvent was removed in vacuo. A solution of 4N HCl (20 mL) and 1,4-dioxane (20 mL) was added to the reaction. After 4 hours, the reaction was neutralized with a saturated solution of sodium bicarbonate then extracted with ethyl acetate. The organic layer was washed with brine and dried over sodium sulfate. The solvent was removed in vacuo to give N-(4-chlorophenyl)-2-phenylpiperazine-1-carboxamide (9.80 g, 31.03 mmol).

Retention time: 2.09 min. (method i)

m/z: (M+H)⁺ 316.

### b) N-(4-Chlorophenyl)-4-(3,4-dioxo-2-(o-tolylamino)cyclobut-1-enyl)-2-phenylpiperazine-1-carboxamide

At 0° C., the o-toluidine (0.102 mL, 0.950 mmol) was added dropwise to a solution of 3,4-dimethoxy-3-cyclobutene-1,2-dione (135 mg, 0.950 mmol) and triethylamine (0.15 mL, 0.950 mmol) in methanol (3 mL). The reaction was allowed to stir at 0° C. for 2 hours before the dropwise addition of N-(4-chlorophenyl)-2-phenylpiperazine-1-carboxamide (300 mg, 0.950 mmol) in methanol (3 mL). After the addition was complete, the reaction was allowed to warm to ambient temperature. The product was purified by reverse-phase HPLC on a Hyperprep HS C18 column, 8 μm, 250×21.2 mm; 20% acetonitrile-50 mM ammonium acetate over 1 min, 20-60% acetonitrile-50 mM ammonium acetate for 34 min, 60-100% acetonitrile for 1 min, 100% acetonitrile for 5 min, 25 ml/min. The product was isolated by lyophilization of the desired fractions to give N-(4-chlorophenyl)-4-(3,4-dioxo-2-(o-tolylamino)cyclobut-1-enyl)-2-phenylpiperazine-1-carboxamide (210 mg, 0.419 mmol).

Retention time: 2.86 min. (method i)

m/z: (M+H)⁺ 501.

### Example 92

### Preparation of −4-(2-(4-Chlorophenylamino)-3,4-dioxocyclobut-1-enyl)-N′-cyano-3-phenyl-N-o-tolylpiperazine-1-carboximidamide

At 0° C., the 4-chloroaniline (0.107 g, 0.844 mmol) was added to a solution of 3,4-dimethoxy-3-cyclobutene-1,2-dione (120 mg, 0.844 mmol) and triethylamine (0.12 mL, 0.844 mmol) in methanol (3 mL). The reaction was allowed to stir at 0° C. for 2 hours before the dropwise addition of —N′-cyano-3-phenyl-N-o-tolylpiperazine-1-carboximidamide (270 mg, 0.844 mmol) in tetrahydrofuran (4 mL). After the addition was complete, the reaction was allowed to warm to ambient temperature. The product was purified by reverse-phase HPLC on a Hyperprep HS C18 column, 8 μm, 250×21.2 mm; 20% acetonitrile-50 mM ammonium acetate over 1 min, 20-60% acetonitrile-50 mM ammonium acetate for 34 min, 60-100% acetonitrile for 1 min, 100% acetonitrile for 5 min, 25 mL/min. The product was isolated by lyophilization of the desired fractions to give -4-(2-(4-chlorophenylamino)-3,4-dioxocyclobut-1-enyl)-N′-cyano-3-phenyl-N-o-tolylpiperazine-1-carboximidamide (45 mg, 0.086 mmol).

Retention time: 2.61 min. (method i)

m/z: (M−H)⁻ 523, 525.

### Example 93

### Preparation of 4-(N-(3-(aminomethyl)phenyl)-N′-cyanocarbamimidoyl)-N-(4-chlorophenyl)-2-phenylpiperazine-1-carboxamide

### a) Benzyl 3-((cyanoimino)(phenoxy)methylamino)benzylcarbamate

Benzyl 3-((cyanoimino)(phenoxy)methylamino)benzylcarbamate was prepared from benzyl 3-aminobenzylcarbamate and diphenylcyanocarboimidate according to general procedure A.

Retention time: 2.03 min. (method d)

m/z: (M−H)⁻ 399.

### b) Benzyl 3-(N′-cyano-3-phenylpiperazine-1-carboximidamido)benzylcarbamate

Benzyl 3-(N′-cyano-3-phenylpiperazine-1-carboximidamido)benzylcarbamate was prepared from benzyl 3-((cyanoimino)(phenoxy)methylamino)benzylcarbamate and 2-phenylpiperazine according to general procedure B.

Retention time: 1.76 min. (method d)

m/z: (M+H)⁺ 469.

### c) Benzyl 3-(4-(4-chlorophenylcarbamoyl)-N′-cyano-3-phenylpiperazine-1-carboximidamido)benzylcarbamate

Benzyl 3-(4-(4-chlorophenylcarbamoyl)-N′-cyano-3-phenylpiperazine-1-carboximidamido)benzylcarbamate was prepared from benzyl 3-(AK-cyano-3-phenylpiperazine-1-carboximidamido)benzylcarbamate and 4-chlorophenylisocyanate according to general procedure C.

Retention time: 2.10 min. (method d)

m/z: (M+H)⁺ 622.

### d) 4-(N-(3-(aminomethyl)phenyl)-N′-cyanocarbamimidoyl)-N-(4-chlorophenyl)-2-phenylpiperazine-1-carboxamide

4-(N-(3-(aminomethyl)phenyl)-N′-cyanocarbamimidoyl)-N-(4-chlorophenyl)-2-phenylpiperazine-1-carboxamide was prepared from benzyl 3-(4-(4-chlorophenylcarbamoyl)-N′-cyano-3-phenylpiperazine-1-carboximidamido)benzylcarbamate using general procedure M.

Retention time: 1.72 min. (method d)

m/z: (M+H)⁺ 488.

### Example 94

### Preparation of 4-(N-(4-(aminomethyl)phenyl)-N′-cyanocarbamimidoyl)-N-(4-chlorophenyl)-2-phenylpiperazine-1-carboxamide

### a) Benzyl 4-((cyanoimino)(phenoxy)methylamino)benzylcarbamate

Benzyl 4-((cyanoimino)(phenoxy)methylamino)benzylcarbamate was prepared from benzyl 4-aminobenzylcarbamate and diphenylcyanocarboimidate according to general procedure A.

Retention time: 1.96 min. (method d)

m/z: (M−H)⁻ 399.

### b) Benzyl 4-(N′-cyano-3-phenylpiperazine-1-carboximidamido)benzylcarbamate

Benzyl 4-(N′-cyano-3-phenylpiperazine-1-carboximidamido)benzylcarbamate was prepared from benzyl 4-((cyanoimino)(phenoxy)methylamino)benzylcarbamate and 2-phenylpiperazine according to general procedure B.

Retention time: 1.73 min. (method d)

m/z: (M+H)⁺ 469.

### c) Benzyl 4-(4-(4-chlorophenylcarbamoyl)-N′-cyano-3-phenylpiperazine-1-carboximidamido)benzylcarbamate

Benzyl 4-(4-(4-chlorophenylcarbamoyl)-N′-cyano-3-phenylpiperazine-1-carboximidamido)benzylcarbamate was prepared from benzyl 4-(N′-cyano-3-phenylpiperazine-1-carboximidamido)benzylcarbamate and 4-chlorophenylisocyanate according to general procedure C.

Retention time: 2.11 min. (method d)

m/z: (M+H)⁺ 622.

### d) 4-(N-(4-(aminomethyl)phenyl)-N′-cyanocarbamimidoyl)-N-(4-chlorophenyl)-2-phenylpiperazine-1-carboxamide

4-(N-(4-(aminomethyl)phenyl)-N′-cyanocarbamimidoyl)-N-(4-chlorophenyl)-2-phenylpiperazine-1-carboxamide was prepared from benzyl 4-(4-(4-chlorophenylcarbamoyl)-N′-cyano-3-phenylpiperazine-1-carboximidamido)benzylcarbamate using general procedure M.

Retention time: 1.76 min. (method d)

m/z: (M+H)⁺ 488.

### Example 95

### Preparation of N′-cyano-3-phenyl-4-(1-phenyl-1H-tetrazol-5-yl)-N-o-tolylpiperazine-1-carboximidamide

To a solution of 5-chloro-1-phenyl-1H-tetrazole (113 mg, 0.626 mmol), 18-crown-6 (248 mg, 0.939 mmol), potassium fluoride (182 mg, 3.131 mmol) and triethylamine (0.175 mL, 1.252 mmol) in 1,4-dioxane (10 ml) was added N′-cyano-3-phenyl-N-o-tolylpiperazine-1-carboximidamide (200 mg, 0.626 mmol). The reaction was stirred at ambient temperature for 18 hours before partitioning with ethyl acetate and brine. The organic layer was dried with sodium sulfate, filtered and the solvent was removed in vacuo. The product was purified by reverse-phase HPLC on a Hyperprep HS C18 column, 8 μm, 250×21.2 mm; 20% acetonitrile-50 mM ammonium acetate over 1 min, 20-60% acetonitrile-50 mM ammonium acetate for 34 min, 60-100% acetonitrile for 1 min, 100% acetonitrile for 5 min, 25 mL/min. The product was isolated by lyophilization of the desired fractions to give N′-cyano-3-phenyl-4-(1-phenyl-1H-tetrazol-5-yl)-N-o-tolylpiperazine-1-carboximidamide (0.020 g, 0.041 mmol).

Retention time: 1.91 min. (method d)

m/z: (M+H)⁺ 464.

### Example 96

### Preparation of 4-(1-(4-chlorophenyl)-1H-tetrazol-5-yl)-N′-cyano-3-phenyl-N-o-tolylpiperazine-1-carboximidamide

### a) 5-Chloro-1-(4-chlorophenyl)-1H-tetrazole

5-Chloro-1-(4-chlorophenyl)-1H-tetrazole was prepared from 4-chlorophenylisocyanide according to Collibee, W. L.; Nakajima, M.; Anselme, Jean-Pierre, Journal of Organic Chemistry, Vol. 60, No. 2., 1995, pp 468-469. RP-HPLC (Method d) Rₜ 2.07 min; MS

m/z: (M+H)⁺ 216.

Retention time: 2.07 min. (method d)

m/z: (M+H)⁺ 216.

### b) 4-(1-(4-chlorophenyl)-1H-tetrazol-5-yl)-N′-cyano-3-phenyl-N-o-tolylpiperazine-1-carboximidamide

To a solution of 5-chloro-1-(4-chlorophenyl)-1H-tetrazole (60 mg, 0.279 mmol), 18-crown-6 (111 mg, 0.419 mmol), potassium fluoride (81 mg, 1.395 mmol) and triethylamine (0.080 mL, 0.558 mmol) in 1,4-dioxane (3 ml) was added N′-cyano-3-phenyl-N-o-tolylpiperazine-1-carboximidamide (89 mg, 0.279 mmol). The reaction was stirred at ambient temperature for 18 hours before partitioning with ethyl acetate and brine. The organic layer was dried with sodium sulfate, filtered and the solvent was removed in vacuo. The product was purified by reverse-phase HPLC on a Hyperprep HS C18 column, 8 μm, 250×21.2 mm; 20% acetonitrile-50 mM ammonium acetate over 1 min, 20-60% acetonitrile-50 mM ammonium acetate for 34 min, 60-100% acetonitrile for 1 min, 100% acetonitrile for 5 min, 25 mL/min. The product was isolated by lyophilization of the desired fractions to give 4-(1-(4-chlorophenyl)-1H-tetrazol-5-yl)-N′-cyano-3-phenyl-N-o-tolylpiperazine-1-carboximidamide (0.033 g, 0.066 mmol).

Retention time: 2.09 min. (method d)

m/z: (M+H)⁺ 498.

### Example 97

### Preparation of 4-(N-(3-amino-2-methylphenyl)-N′-cyanocarbamimidoyl)-N-(3-fluorophenyl)-3-isopropylpiperazine-1-carboxamide

4-(N′-cyano-N-(2-methyl-3-nitrophenyl)carbamimidoyl)-N-(3-fluorophenyl)-3-isopropylpiperazine-1-carboxamide (1.90 g, 4.06 mmol) and 10% palladium on carbon (0.30 g, 0.28 mmol) in methanol (40 ml) was shaken under hydrogen (60 psi) for 18 hours before the reaction was filtered through celite. The solvent is removed in vacuo before the product was purified by reverse-phase HPLC on a Hyperprep HS C18 column, 8 μm, 250×21.2 mm; 20% acetonitrile-50 mM ammonium acetate over 1 min, 20-60% acetonitrile-50 mM ammonium acetate for 34 min, 60-100% acetonitrile for 1 min, 100% acetonitrile for 5 min, 25 mL/min. The product was isolated by lyophilization of the desired fractions to give 4-(N-(3-amino-2-methylphenyl)-N′-cyanocarbamimidoyl)-N-(3-fluorophenyl)-3-isopropylpiperazine-1-carboxamide (1.20 g, 2.74 mmol).

Retention time: 1.64 min. (method d)

m/z: (M+H)⁺ 438.

### Example 98

### Preparation of 4-(N′-cyano-N-(2-(2-(diethylamino)ethylamino)quinolin-5-yl)carbamimidoyl)-N-(3-fluorophenyl)-3-isopropylpiperazine-1-carboxamide

At 0° C., N,N-dimethylcarbamoyl chloride (0.019 mL, 0.20 mmol) was added to a solution of 5-(N′-cyano-4-(3-fluorophenylcarbamoyl)-2-isopropylpiperazine-1-carboximidamido)quinoline 1-oxide (65 mg, 0.14 mmol) in dichloromethane (0.8 mL). The reaction was allowed to stir at 0° C. for 1 hour before the addition of N,N-diethyl-1,2-ethanediamine (0.20 mL, 1.00 mmol). After the addition was complete, the reaction was allowed to warm to ambient temperature. The product was purified by reverse-phase HPLC on a Hyperprep HS C18 column, 8 μm, 250×21.2 mm; 20% acetonitrile-50 mM ammonium acetate over 1 min, 20-50% acetonitrile-50 mM ammonium acetate for 34 min, 50-100% acetonitrile for 1 min, 100% acetonitrile for 5 min, 25 mL/min. The product was isolated by lyophilization of the desired fractions to give 4-(N′-cyano-N-(2-(2-(diethylamino)ethylamino)quinolin-5-yl)carbamimidoyl)-N-(3-fluorophenyl)-3-isopropylpiperazine-1-carboxamide (8 mg, 0.01 mmol).

Retention time: 1.81 min. (method d)

m/z: (M+H)⁺ 574.

### Example 99

### Preparation of 2-(4-(N′-cyano-N-(quinolin-5-yl)carbamimidoyl)-3-isopropylpiperazin-1-yl)thiazole-5-carboxylic acid

To a solution of ethyl 2-(4-(N′-cyano-N-(quinolin-5-yl)carbamimidoyl)-3-isopropylpiperazin-1-yl)thiazole-5-carboxylate (1.00 g, 2.09 mmol) in methanol (4 mL) was added 50% aqueous solution of sodium hydroxide. The reaction was stirred at ambient temperature for 6 hours before neutralization with 1 N HCl. The reaction was partitioned with ethyl acetate and brine. The organic layer was dried with sodium sulfate, filtered and the solvent was removed in vacuo. The product was purified by reverse-phase HPLC on a Hyperprep HS C18 column, 8 μm, 250×21.2 mm; 20% acetonitrile-50 mM ammonium acetate over 1 min, 20-50% acetonitrile-50 mM ammonium acetate for 34 min, 50-100% acetonitrile for 1 min, 100% acetonitrile for 5 min, 25 mL/min. The product was isolated by lyophilization of the desired fractions to give 2-(4-(N′-cyano-N-(quinolin-5-yl)carbamimidoyl)-3-isopropylpiperazin-1-yl)thiazole-5-carboxylic acid (80 mg, 0.178 mmol).

Retention time: 0.70 min. (method d)

m/z: (M+H)⁺ 450.

### Example 100

### Preparation of 2-(4-(N′-cyano-N-(quinolin-5-yl)carbamimidoyl)-3-isopropylpiperazin-1-yl)-N,N-dimethylthiazole-5-carboxamide

2-(4-(N′-cyano-N-(quinolin-5-yl)carbamimidoyl)-3-isopropylpiperazin-1-yl)-N,N-dimethylthiazole-5-carboxamide was prepared from 2-(4-(N′-cyano-N-(quinolin-5-yl)carbamimidoyl)-3-isopropylpiperazin-1-yl)thiazole-5-carboxylic acid and dimethylamine using general procedure D.

Retention time: 1.26 min. (method d)

m/z: (M+H)⁺ 477.

### Example 101

### Preparation of 2-(4-(N′-cyano-N-(quinolin-5-yl)carbamimidoyl)-3-isopropylpiperazin-1-yl)thiazole-5-carboxamide

2-(4-(N′-cyano-N-(quinolin-5-yl)carbamimidoyl)-3-isopropylpiperazin-1-yl)thiazole-5-carboxamide was prepared from 2-(4-(N′-cyano-N-(quinolin-5-yl)carbamimidoyl)-3-isopropylpiperazin-1-yl)thiazole-5-carboxylic acid and ammonium hydroxide using general procedure D.

Retention time: 1.05 min. (method d)

m/z: (M+H)⁺ 449.

### Example 102

### Preparation of N′-cyano-4-(3,4-dimethoxybenzoyl)-3-phenyl-N-(quinolin-5-yl)piperazine-1-carboximidamide

### a) (3,4-Dimethoxyphenyl)(2-phenylpiperazin-1-yl)methanone

At 0° C., a solution of 3,4-dimethoxybenzoyl chloride (0.191 g, 0.953 mmol) in dichloromethane (4 ml) was added dropwise to a solution of 3-phenylpiperazine-1-carboxylic acid tert-butyl ester (0.250 g, 0.953 mmol) in dichloromethane (4 mL). The reaction was allowed to warm to ambient temperature before the solvent was removed in vacuo. A solution of 4N HCl (3 mL) and 1,4-dioxane (3 mL) was added to the reaction. After 4 hours, the reaction was neutralized with a saturated solution of sodium bicarbonate then extracted with ethyl acetate. The organic layer was washed with brine and dried over sodium sulfate. The solvent was removed in vacuo to give (3,4-dimethoxyphenyl)(2-phenylpiperazin-1-yl)methanone (0.22 g, 0.674 mmol).

Retention time: 1.35 min. (method i)

m/z: (M+H)⁺ 327.

### b) N′-cyano-4-(3,4-dimethoxybenzoyl)-3-phenyl-N-(quinolin-5-yl)piperazine-1-carboximidamide

N′-cyano-4-(3,4-dimethoxybenzoyl)-3-phenyl-N-(quinolin-5-yl)piperazine-1-carboximidamide was prepared from (3,4-dimethoxyphenyl)(2-phenylpiperazin-1-yl)methanone and 5-isothiocyanatoquinoline using general procedure G.

Retention time: 1.80 min. (method i)

m/z: (M+H)⁺ 521.

### Preparation 7: Preparation of Benzyl 5-isothiocyanato-3,4-dihydroisoquinoline-2(1H)-carboxylate

Benzyl 5-isothiocyanato-3,4-dihydroisoquinoline-2(1H)-carboxylate was prepared from benzyl 5-amino-3,4-dihydroisoquinoline-2(1H)-carboxylate using general procedure F. ¹HNMR (DMSO-d₆, 400 MHz) δ 7.33 (m, 4H), 7.25 (m, 4H), 5.13 (s, 2H), 4.60 (bs, 2H), 3.70 (bs, 2H), 2.81 (m, 2H).

### Example 103

### Preparation of 4-(4-Chlorobenzyl)-N′-cyano-3-phenyl-N-o-tolylpiperazine-1-carboximidamide

A solution of 4-chlorobenzylchloride (76 mg, 0.470 mmol), N′-cyano-3-phenyl-N-o-tolylpiperazine-1-carboximidamide (75 mg, 0.235 mmol) and triethylamine (0.07 mL, 0.470 mmol) in acetonitrile (3 mL) was refluxed for 18 hours. The product was purified by reverse-phase HPLC on a Hyperprep HS C18 column, 8 μm, 250×21.2 mm; 20% acetonitrile-50 mM ammonium acetate over 1 min, 20-70% acetonitrile-50 mM ammonium acetate for 34 min, 70-100% acetonitrile for 1 min, 100% acetonitrile for 5 min, 25 mL/min. The product was isolated by lyophilization of the desired fractions to give 4-(4-chlorobenzyl)-N′-cyano-3-phenyl-N-o-tolylpiperazine-1-carboximidamide (16 mg, 0.036 mmol).

Retention time: 3.78 min. (method i)

m/z: (M+H)⁺ 444.

### Example 104

### Preparation of 4-(3-(4-Chlorophenyl)propyl)-N′-cyano-3-phenyl-N-o-tolylpiperazine-1-carboximidamide

A solution of 1-chloro-3-(4-chlorophenyl)propane (88 mg, 0.470 mmol), N′-cyano-3-phenyl-N-o-tolylpiperazine-1-carboximidamide (75 mg, 0.235 mmol) and triethylamine (0.07 mL, 0.470 mmol) in acetonitrile (3 mL) was refluxed for 18 hours. The product was purified by reverse-phase HPLC on a Hyperprep HS C18 column, 8 μm, 250×21.2 mm; 20% acetonitrile-50 mM ammonium acetate over 1 min, 20-70% acetonitrile-50 mM ammonium acetate for 34 min, 70-100% acetonitrile for 1 min, 100% acetonitrile for 5 min, 25 mL/min. The product was isolated by lyophilization of the desired fractions to give 4-(3-(4-chlorophenyl)propyl)-N′-cyano-3-phenyl-N-o-tolylpiperazine-1-carboximidamide (26 mg, 0.055 mmol).

Retention time: 3.92 min. (method i)

m/z: (M+H)⁺ 472.

### Example 105

### Preparation of 4-(5-Amino-1-phenyl-1H-1,2,4-triazol-3-yl)-N-(4-chlorophenyl)-2-phenylpiperazine-1-carboxamide

### a) Phenyl 4-(4-chlorophenylcarbamoyl)-N-cyano-3-phenylpiperazine-1-carbimidate

Phenyl 4-(4-chlorophenylcarbamoyl)-N-cyano-3-phenylpiperazine-1-carbimidate was prepared from N-(4-chlorophenyl)-2-phenylpiperazine-1-carboxamide and diphenylcyanocarboimidate using General Procedure A.

Retention time: 2.17 min. (method d)

m/z: (M+H)⁺ 460.

### b) 4-(5-Amino-1-phenyl-1H-1,2,4-triazol-3-yl)-N-(4-chlorophenyl)-2-phenylpiperazine-1-carboxamide

Phenyl 4-(4-chlorophenylcarbamoyl)-N-cyano-3-phenylpiperazine-1-carbimidate (0.54 g, 1.17 mmol) and phenylhydrazine (0.70 mL, 7.10 mmol) were stirred at ambient temperature for 18 hours before the reaction was diluted with ethyl acetate and washed with 1 N HCl followed by brine The organic layer was dried with sodium sulfate, filtered and the solvent was removed in vacuo. The product was purified by reverse-phase HPLC on a Hyperprep HS C18 column, 8 gm, 250×21.2 mm; 20% acetonitrile-50 mM ammonium acetate over 1 min, 20-60% acetonitrile-50 mM ammonium acetate for 34 min, 60-100% acetonitrile for 1 min, 100% acetonitrile for 5 min, 25 mL/min. The product was isolated by lyophilization of the desired fractions to give 4-(5-amino-1-phenyl-1H-1,2,4-triazol-3-yl)-N-(4-chlorophenyl)-2-phenylpiperazine-1-carboxamide (35 mg, 0.074 mmol).

Retention time: 1.92 min. (method d)

m/z: (M+H)⁺ 474.

### Preparation 8: Preparation of 2-Chloro-5-isothiocyanatoquinoline

### a) Quinolin-5-ylcarbamic acid tert-butyl ester

A mixture of 5-aminoquinoline (3.00 g, 20.81 mmol), di-tert-butyl carbonate (9.54 g, 43.71 mmol) and sodium carbonate (4.41 g, 41.61 mmol) in tetrahydrofuran (50 mL) was refluxed for 18 hours before the reaction was diluted with ethyl acetate and washed with brine. The organic layer was dried with sodium sulfate, filtered and the solvent was removed in vacuo. The product was purified by chromatography on silica gel using 20-30% ethyl acetate in heptanes to give quinolin-5-ylcarbamic acid tert-butyl ester (4.45 g, 18.22 mmol).

Retention time: 1.68 min. (method d)

m/z: (M+H)⁺ 245.

### b) (1-Oxy-quinolin-5-yl)carbamic acid tert-butyl ester

(1-Oxy-quinolin-5-yl)carbamic acid tert-butyl ester was prepared from quinolin-5-ylcarbamic acid tert-butyl ester using general procedure I.

Retention time: 1.20 min. (method d)

m/z: (M+H)⁺ 261.

### c) 2-Chloroquinolin-5-yl-amine

A solution of (1-oxy-quinolin-5-yl)carbamic acid tert-butyl ester (2.00 g, 7.68 mmol) and phosphorousoxychloride (2.80 mL, 30.59 mmol) in chloroform (15 mL) was refluxed for 18 hours before it was quenched with ice water. The reaction was partitioned with ethyl acetate and brine then subsequently washed with a saturated solution of sodium bicarbonate. The organic layer was dried with sodium sulfate, filtered and the solvent was removed in vacuo. The product was purified by chromatography on silica gel using 10-20% ethyl acetate in heptanes to give 2-chloroquinolin-5-yl-amine (0.44 g, 2.46 mmol).

Retention time: 1.41 min. (method d)

m/z: (M+H)⁺ 179.

### d) 2-Chloro-5-isothiocyanatoquinoline

2-Chloro-5-isothiocyanatoquinoline was prepared from 2-chloroquinolin-5-yl-amine using general procedure F.

Retention time: 2.70 min. (method d)

m/z: (M+H)⁺ 219.

### Preparation 9: Preparation of 2-Fluoro-5-isothiocyanatoquinoline

### a) 2-Fluoroquinolin-5-yl-amine

2-Fluoroquinolin-5-yl-amine was prepared from 2-chloroquinolin-5-yl-amine according to Heterocycles, 34(8), 1507-1510 (1992).

Retention time: 1.20 min. (method d)

m/z: (M+H)⁺ 163.

### b) 2-Fluoro-5-isothiocyanatoquinoline

2-Fluoro-5-isothiocyanatoquinoline was prepared from 2-fluoroquinolin-5-yl-amine using general procedure F.

Retention time: 2.25 min. (method d)

m/z: (M−H)⁻ 203.

### Example 106

### N′-cyano-4-methyl-2-phenyl-N-(quinolin-5-yl)piperazine-1-carboximidamide

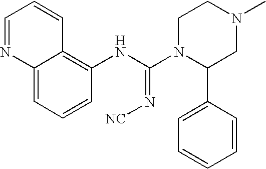

a) To a suspension of 5-aminoquinoline (Aldrich) 5.00 g (34.7 mmol) in 40 mL of dichloromethane and a solution of 20.4 g of sodium bicarbonate in 75 mL of water was added drop wise at 0° C. a solution of thiophosgen in 20 mL of dichloromethane. The reaction was stirred for 2 h at 0° C., the organic layer separated, washed with saturated aqueous NaHCO₃ and brine. The organic layer was dried over MgSO₄ and concentrated to afford 6.38 g of the 5-isothicyanatoquinoline as a light yellow solid.

MS (ESI⁺) m/z 187 (M+H)⁺; ¹H NMR (300 MHz, DMSO-d₆) δ ppm 7.73 (dd, J=8.48, 4.41 Hz, 1H) 7.77-7.85 (m, 2H) 8.06 (dd, J=7.12, 2.37 Hz, 1H) 8.46 (d, J=8.48 Hz, 1H) 9.03 (dd, J=4.07, 1.70 Hz, 1H)

b) A mixture of product from part A 6.38 g (34.3 mmol) and sodium hydrogencyanamide 2.19 g (34.3 mmol) in 30 mL of dimethylformamide was stirred at room temperature for 1 h. Methyl iodide 2.13 mL (34.3 mmol) was added at 0° C. and the reaction was stirred at room temperature for 2 h. The reaction was poured into water and stirred for 20 min. The orange precipitate was filtered and washed with water. The precipitate was purified by flash chromatography on SiO₂ (EtOAc:CH₂Cl₂, 1:1) to obtain 5.77 g of 5-isothiocyanatoquinoline as an orange solid.

MS (ESI⁺) m/z 243 (M+H)⁺; ¹H NMR (300 MHz, DMSO-d₆) δ ppm 2.72 (s, 3H) 7.56-7.62 (m, 1H) 7.63 (dd, J=8.14, 3.73 Hz, 1H) 7.82 (dd, J=8.48, 7.46 Hz, 1H) 8.06 (d, J=8.48 Hz, 1H) 8.30 (d, J=8.48 Hz, 1H) 8.97 (dd, J=4.24, 1.53 Hz, 1H) 10.55 (s, 1H).

c) A mixture of product from part B (80 mg, 0.33 mmol), 1-methyl-3-phenyl-piperazine (Aldrich) (58 mg, 0.33 mmol) and mercuric acetate (105 mg, 0.33 mmol) in 5 mL of anhydrous tetrahydrofuran was stirred at room temperature for 2 h. The precipitate was filtered through a pad of Celite. The filtrate was purified by flash chromatography on SiO₂ (MeOH:CH₂Cl₂, 3%) to obtain N′-cyano-4-methyl-2-phenyl-N-(quinolin-5-yl)piperazine-1-carboximidamide as a white solid.

MS (ESI⁺) m/z 371 (M+H)⁺; ¹H NMR (300 MHz, DMSO-d₆) δ ppm 2.08-2.16 (m, 1H) 2.22 (s, 3H) 2.42 (dd, J=12.04, 3.90 Hz, 1H) 2.78 (d, J=10.51 Hz, 1H) 3.12-3.26 (m, 1H) 3.39 (d, J=11.87 Hz, 1H) 4.00 (d, J=13.90 Hz, 1H) 5.57 (s, 1H) 7.26-7.34 (m, 1H) 7.37-7.46 (m, 1H) 7.46-7.52 (m, 1H) 7.57 (dd, J=8.48, 4.07 Hz, 1H) 7.71 (dd, J=8.48, 7.46 Hz, 1H) 7.87 (d, J=8.48 Hz, 1H) 8.35 (d, J=8.14 Hz, 1H) 8.92 (dd, J=4.24, 1.53 Hz, 1H) 9.51 (s, 1H)

### Example 107

### (S)-4-benzyl-N′-cyano-2-phenyl-N-(quinolin-5-yl)piperazine-1-carboximidamide

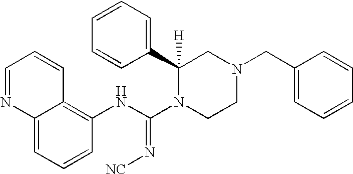

The compound from part B (100 mg, 0.413 mmol)) was reacted with (R)—N⁴-benzyl-2-phenylpiperazine (Anaspec) (104 mg, 0.413 mmol) and mercuric acetate (132 mg, 0.413 mmol) according to the method of part C to provide (S)-4-benzyl-N′-cyano-2-phenyl-N-(quinolin-5-yl)piperazine-1-carboximidamide as a white solid.

MS (ESI⁺) m/z 447 (M+H)⁺; ¹H NMR (300 MHz, DMSO-d₆) δ ppm 2.16-2.30 (m, 1H) 2.51-2.56 (m, 1H) 2.83 (d, J=11.87 Hz, 1H) 3.19-3.27 (m, 1H) 3.38 (d, J=12.21 Hz, 1H) 3.46-3.63 (m, 2H) 4.01 (d, J=12.88 Hz, 1H) 5.57 (s, 1H) 7.21-7.37 (m, 5H) 7.44 (s, 3H) 7.56 (dd, J=8.48, 4.41 Hz, 1H) 7.70 (dd, J=8.48, 7.46 Hz, 1H) 7.88 (d, J=8.48 Hz, 1H) 8.34 (d, J=8.48 Hz, 1H) 8.92 (dd, J=4.41, 1.70 Hz, 1H) 9.62 (s, 1H)
Schemes and Tables

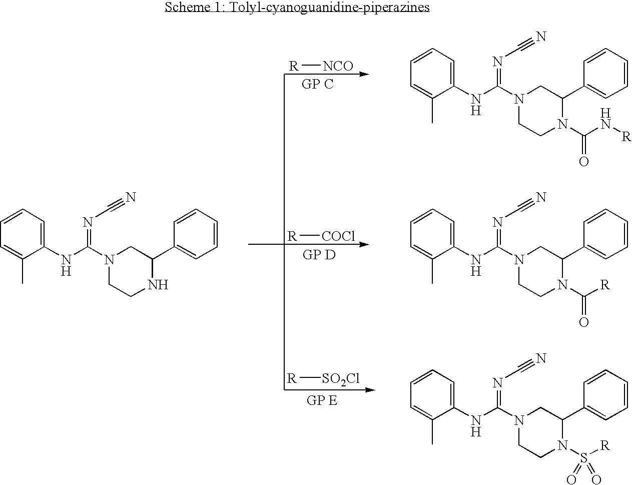

The compounds in table 1 were made according to either general procedure C, D or E as illustrated in scheme 1. The specific procedure used for each compound is listed in table 1.

TABLE 1
Tolyl-cyanoguanidine-piperazines

| | | | | Rt/min | |
|---|---|---|---|---|---|
| Ex # | Reagent | GP | Product | (method) | m/z |
| 1.1 | 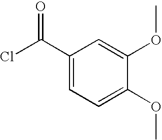 | D | 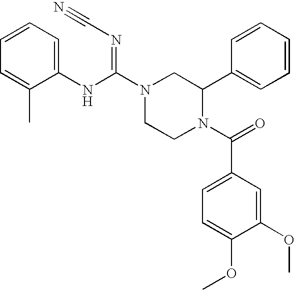 | 2.25 (i) | 584 (M + H)⁺ |
| 1.2 | 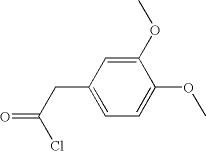 | D | 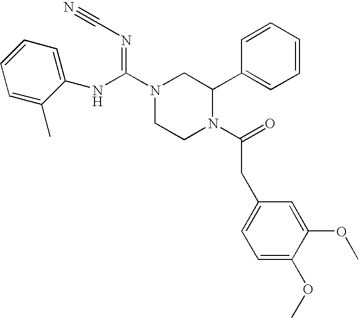 | 1.92 (i) | 498 (M + H)⁺ |
| 1.3 | 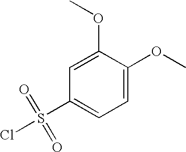 | E | 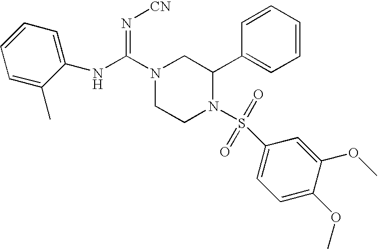 | 2.58 (i) | 520 (M + H)⁺ |
| 1.4 | 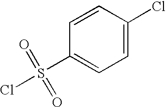 | E | 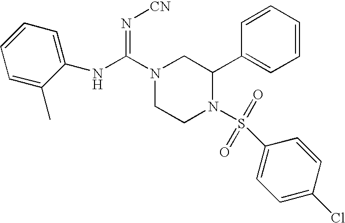 | 3.12 (i) | 494 (M + H)⁺ |
| 1.5 | 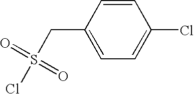 | E | 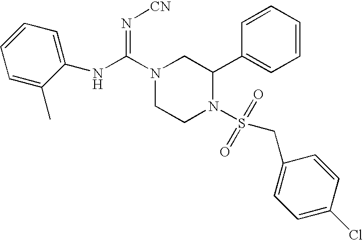 | 3.00 (i) | 508 (M + H)⁺ |
| 1.6 | 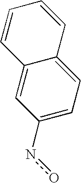 | C | 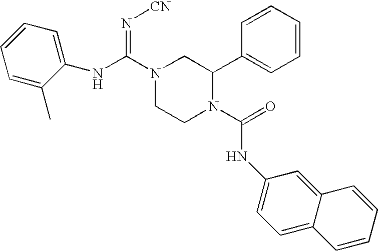 | 2.92 (i) | 489 (M + H)⁺ |
| 1.7 | 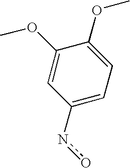 | C | 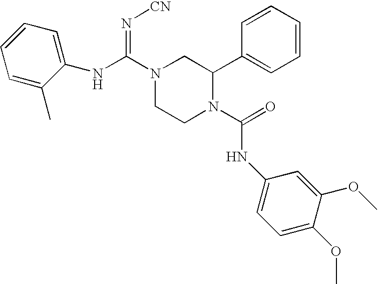 | 2.23 (i) | 499 (M + H)⁺ |
| 1.8 | 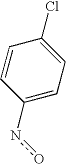 | C | 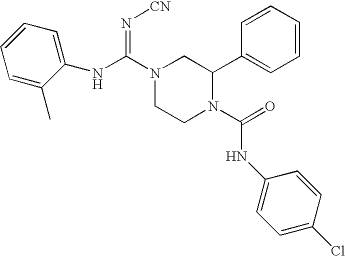 | 2.85 (i) | 473 (M + H)⁺ |
| 1.9 | 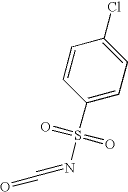 | C | 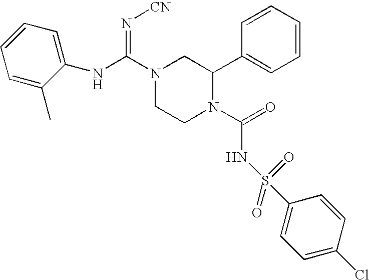 | 1.82 (i) | 537 (M + H)⁺ |
| 1.10 | 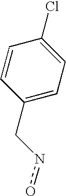 | C | 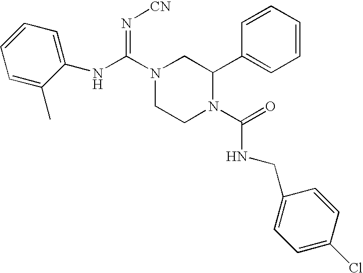 | 2.65 (i) | 487 (M + H)⁺ |
| 1.11 | 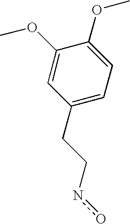 | C | 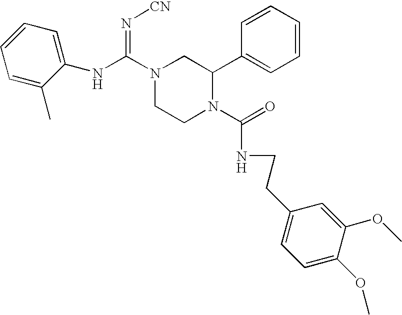 | 2.32 (i) | 527 (M + H)⁺ |
| 1.12 | 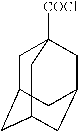 | D | 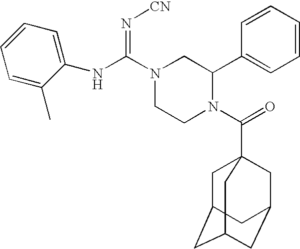 | 3.53 (i) | 496 (M + H)⁺ |
| 1.13 | 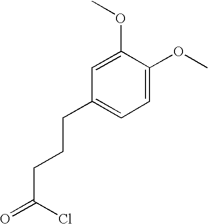 | D | 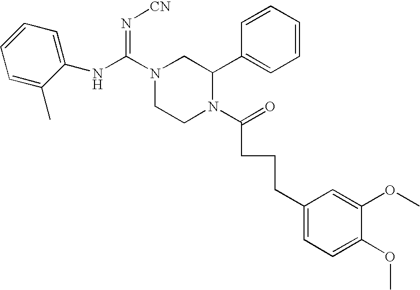 | 2.50 (i) | 526 (M + H)⁺ |
| 1.14 | 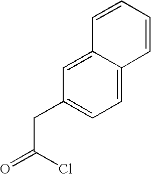 | D | 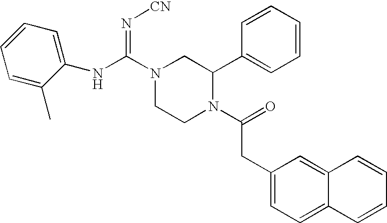 | 2.91 (i) | 488 (M + H)⁺ |
| 1.15 | 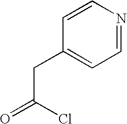 | D | 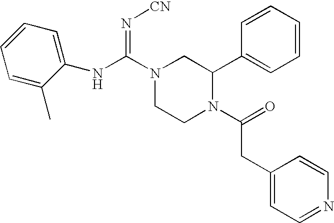 | 1.85 (i) | 439 (M + H)⁺ |
| 1.16 | 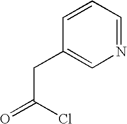 | D | 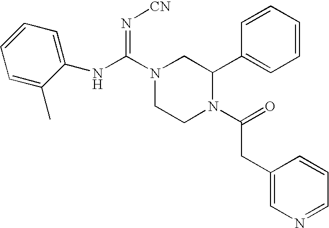 | 1.96 (i) | 439 (M + H)⁺ |
| 1.17 | 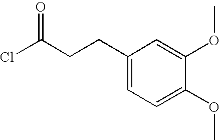 | D | 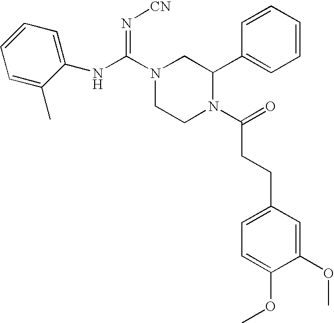 | 2.53 (i) | 512 (M + H)⁺ |
| 1.18 | 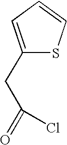 | D | 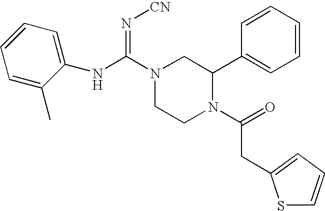 | 2.62 (i) | 444 (M + H)⁺ |
| 1.19 | 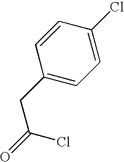 | D | 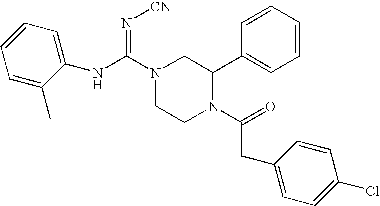 | 2.92 (i) | 472 (M + H)⁺ |
| 1.20 | 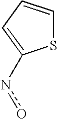 | C | 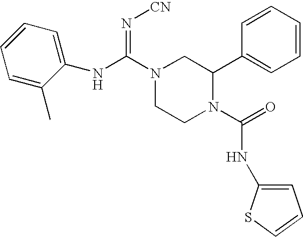 | 2.44 (i) | 445 (M + H)⁺ |
| 1.21 | 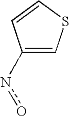 | C | 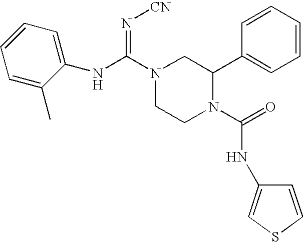 | 2.42 (i) | 445 (M + H)⁺ |
| 1.22 | 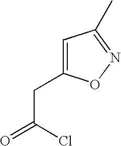 | D | 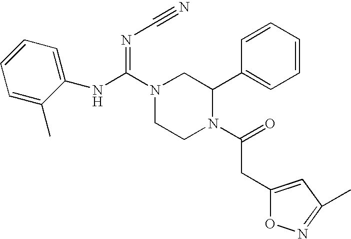 | 2.07 (i) | 443 (M + H)⁺ |
| 1.23 | 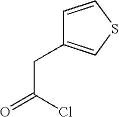 | D | 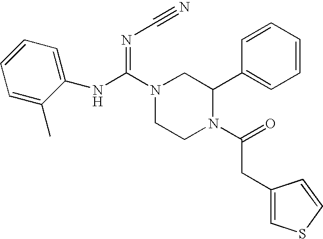 | 2.45 (i) | 444 (M + H)⁺ |
| 1.24 | 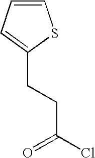 | D | 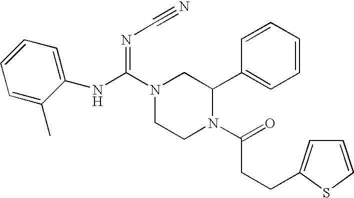 | 2.65 (i) | 458 (M + H)⁺ |
| 1.25 | 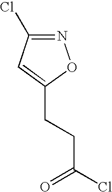 | D | 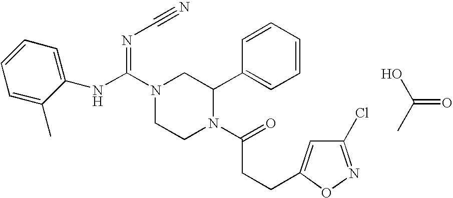 | 2.50 (i) | 477 (M + H)⁺ |
| 1.26 | 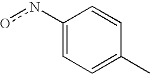 | C | 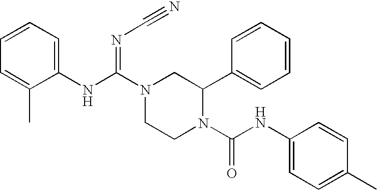 | 2.42 (k) | 451 (M − H)⁻ |
| 1.27 | 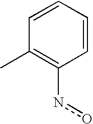 | C | 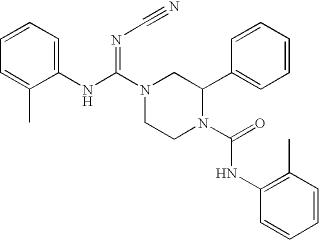 | 2.34 (k) | 451 (M − H)⁻ |
| 1.28 | 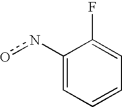 | C | 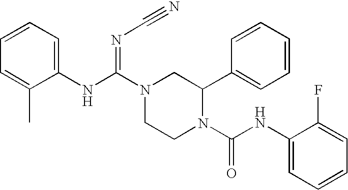 | 2.32 (k) | 455 (M − H)⁻ |
| 1.29 | 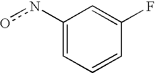 | C | 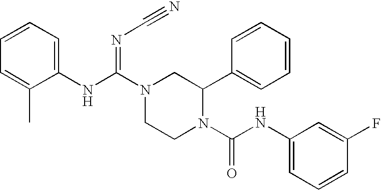 | 2.53 (i) | 457 (M + H)⁺ |
| 1.30 | 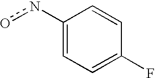 | C | 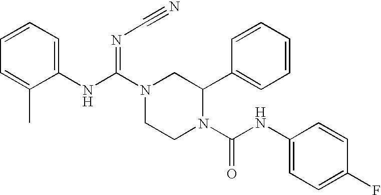 | 2.34 (K) | 455 (M − H)⁻ |
| 1.31 | 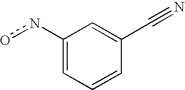 | C | 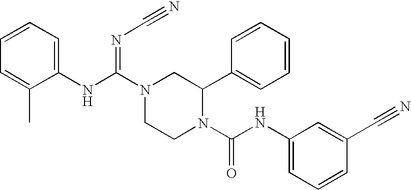 | 2.37 (i) | 464 (M + H)⁺ |
| 1.32 | 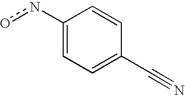 | C | 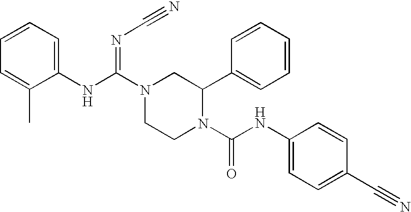 | 2.29 (k) | 462 (M − H)⁻ |
| 1.33 | 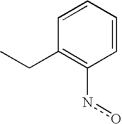 | C | 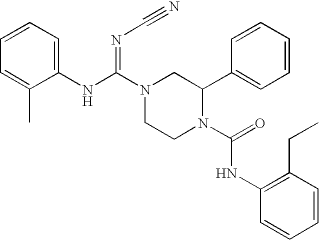 | 2.44 (k) | 465 (M − H)⁻ |
| 1.34 | 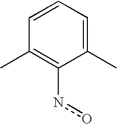 | C | 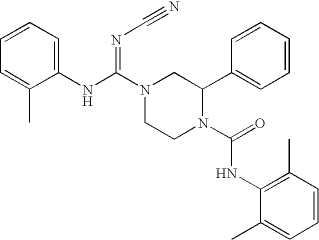 | 2.34 (k) | 465 (M − H)⁻ |
| 1.35 | 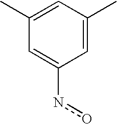 | C | 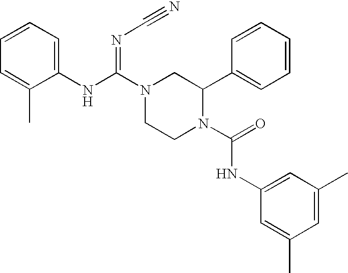 | 2.54 (k) | 465 (M − H)⁻ |
| 1.36 | 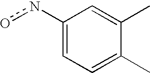 | C | 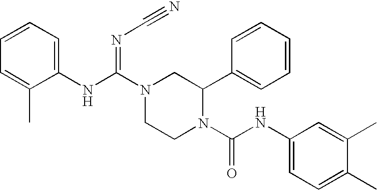 | 2.51 (k) | 465 (M − H)⁻ |
| 1.37 | 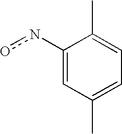 | C | 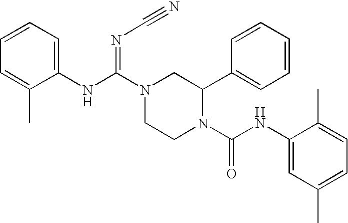 | 2.47 (k) | 465 (M − H)⁻ |
| 1.38 | 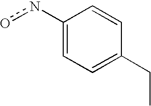 | C | 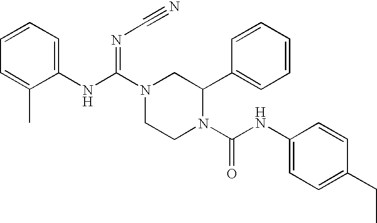 | 2.65 (k) | 465 (M − H)⁻ |
| 1.39 | 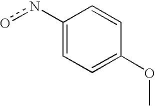 | C | 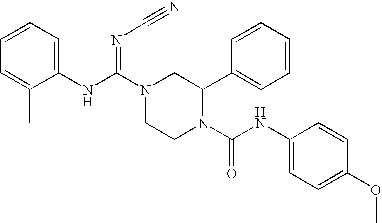 | 2.25 (k) | 467 (M − H)⁻ |
| 1.40 | 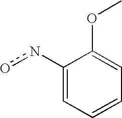 | C | 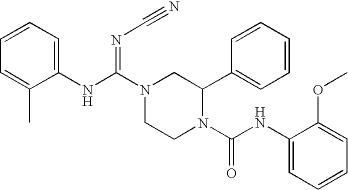 | 2.41 (k) | 467 (M − H)⁻ |
| 1.41 | 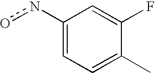 | C | 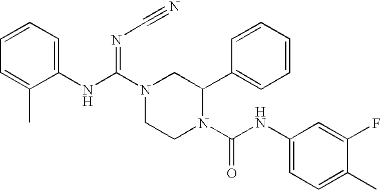 | 2.52 (k) | 469 (M − H)⁻ |
| 1.42 | 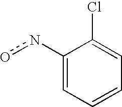 | C | 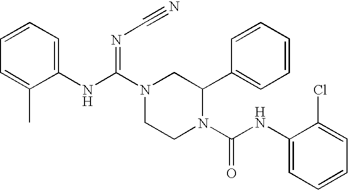 | 2.48 (k) | 471 (M − H)⁻ |
| 1.43 | 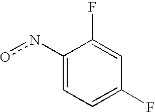 | C | 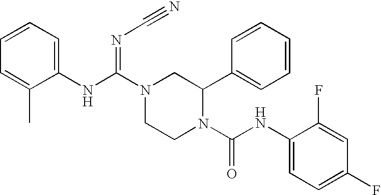 | 2.34 (k) | 473 (M − H)⁻ |
| 1.44 | 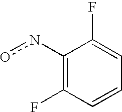 | C | 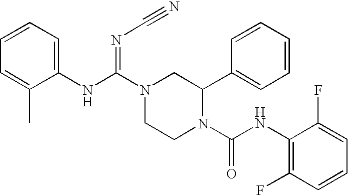 | 2.22 (k) | 473 (M − H)⁻ |
| 1.45 | 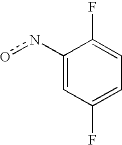 | C | 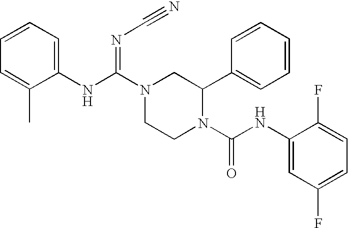 | 2.44 (k) | 473 (M − H)⁻ |
| 1.46 | 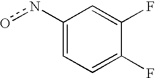 | C | 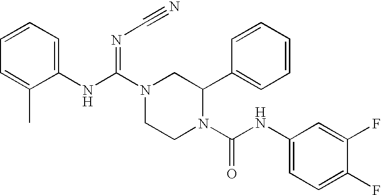 | 2.45 (k) | 473 (M − H)⁻ |
| 1.47 | 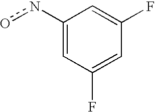 | C | 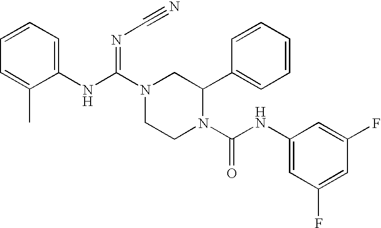 | 2.52 (k) | 473 (M − H)⁻ |
| 1.48 | 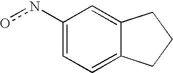 | C | 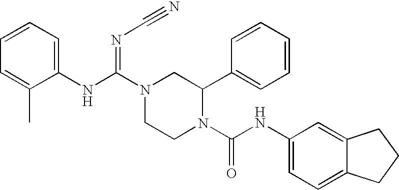 | 2.58 (k) | 477 (M − H)⁻ |
| 1.49 | 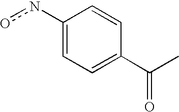 | C | 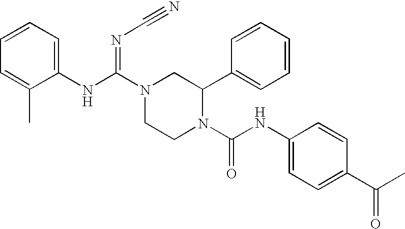 | 2.20 (k) | 479 (M − H)⁻ |
| 1.50 | 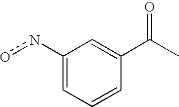 | C | 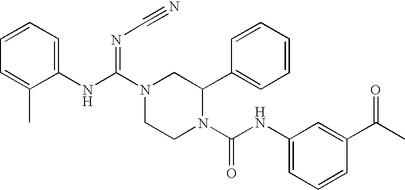 | 2.22 (k) | 479 (M − H)⁻ |
| 1.51 | 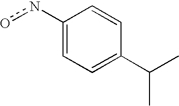 | C | 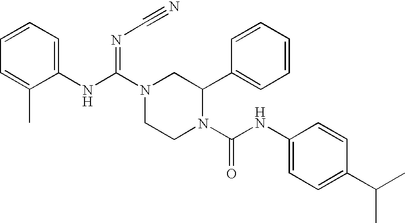 | 2.68 (k) | 479 (M − H)⁻ |
| 1.52 | 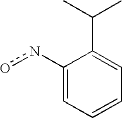 | C | 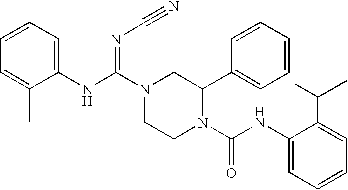 | 2.53 (k) | 479 (M − H)⁻ |
| 1.53 | 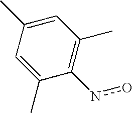 | C | 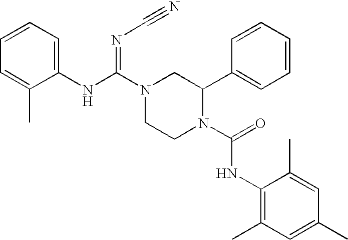 | 2.47 (k) | 479 (M − H)⁻ |
| 1.54 | 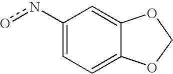 | C | 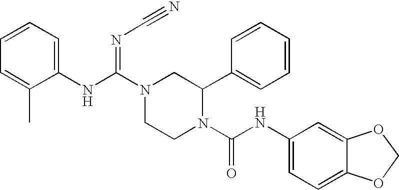 | 2.25 (k) | 481 (M − H)⁻ |
| 1.55 | 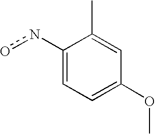 | C | 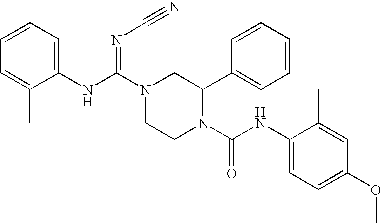 | 2.25 (k) | 481 (M − H)⁻ |
| 1.56 | 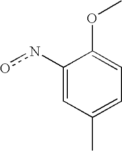 | C | 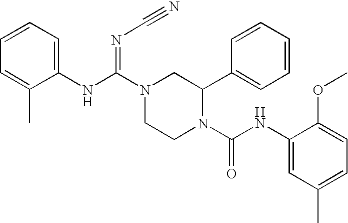 | 2.51 (k) | 481 (M − H)⁻ |
| 1.57 | 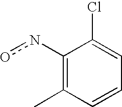 | C | 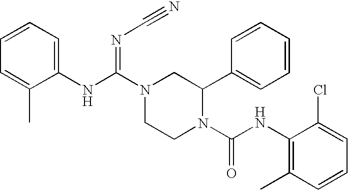 | 2.32 (k) | 485 (M − H)⁻ |
| 1.58 | 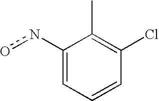 | C | 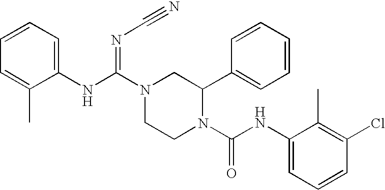 | 2.48 (k) | 485 (M − H)⁻ |
| 1.59 | 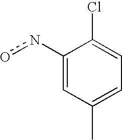 | C | 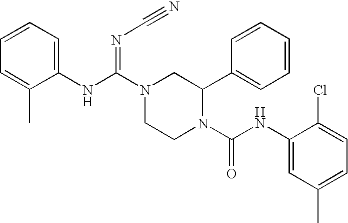 | 2.62 (k) | 485 (M − H)⁻ |
| 1.60 | 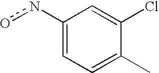 | C | 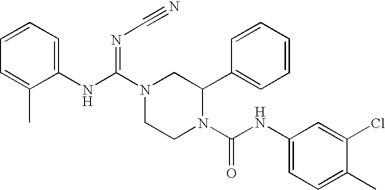 | 2.58 (k) | 485 (M − H)⁻ |
| 1.61 | 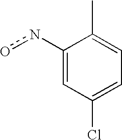 | C | 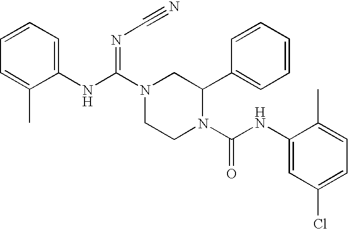 | 2.54 (k) | 485 (M − H)⁻ |
| 1.62 | 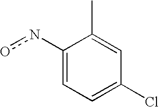 | C | 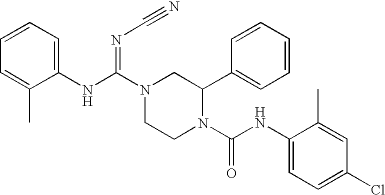 | 2.51 (k) | 485 (M − H)⁻ |
| 1.63 | 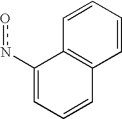 | C | 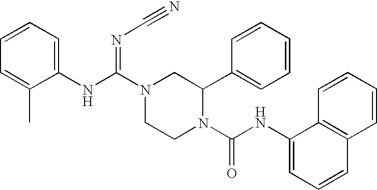 | 2.39 (k) | 487 (M − H)⁻ |
| 1.64 | 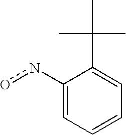 | C | 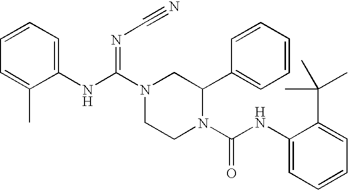 | 2.56 (k) | 493 (M − H)⁻ |
| 1.65 | 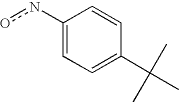 | C | 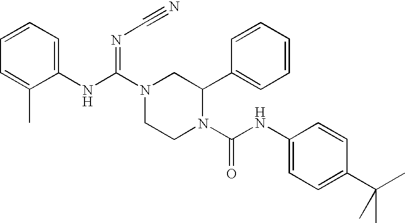 | 2.74 (k) | 493 (M − H)⁻ |
| 1.66 | 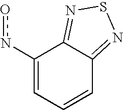 | C | 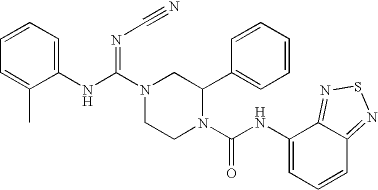 | 2.48 (k) | 495 (M − H)⁻ |
| 1.67 | 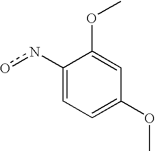 | C | 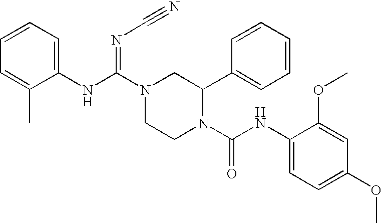 | 2.33 (k) | 497 (M − H)⁻ |
| 1.68 | 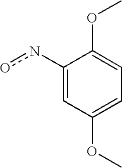 | C | 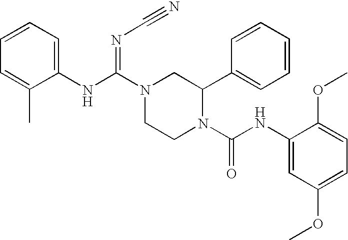 | 2.47 (k) | 497 (M − H)⁻ |
| 1.69 | 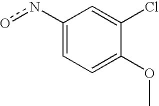 | C | 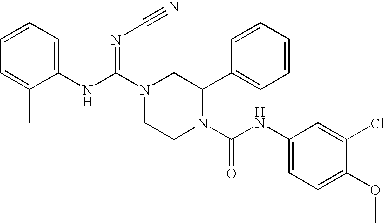 | 2.38 (k) | 501 (M − H)⁻ |
| 1.70 | 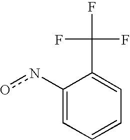 | C | 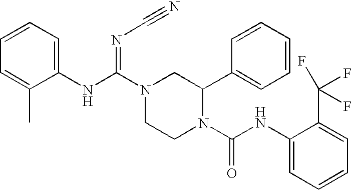 | 2.41 (k) | 505 (M − H)⁻ |
| 1.71 | 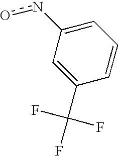 | C | 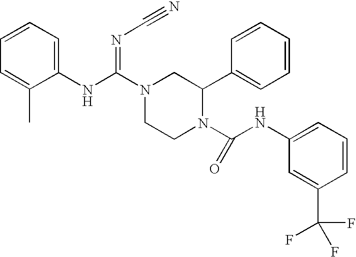 | 2.56 (k) | 505 (M − H)⁻ |
| 1.72 | 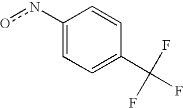 | C | 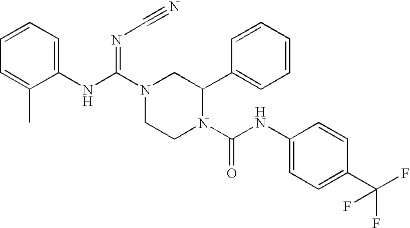 | 2.58 (k) | 505 (M − H)⁻ |
| 1.73 | 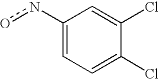 | C | 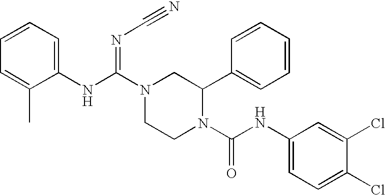 | 2.65 (k) | 505 (M − H)⁻ |
| 1.74 | 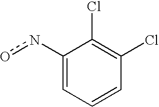 | C | 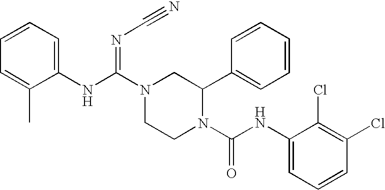 | 2.67 (k) | 505 (M − H)⁻ |
| 1.75 | 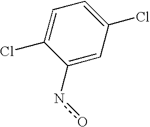 | C | 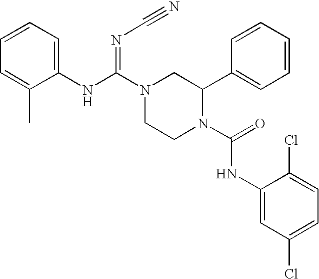 | 2.70 (k) | 505 (M − H)⁻ |
| 1.76 | 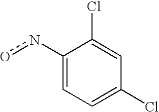 | C | 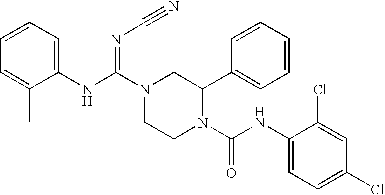 | 2.71 (k) | 505 (M − H)⁻ |
| 1.77 | 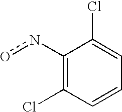 | C | 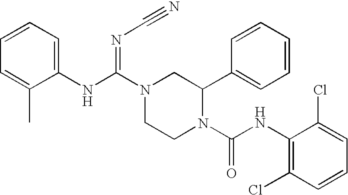 | 2.34 (k) | 505 (M − H)⁻ |
| 1.78 | 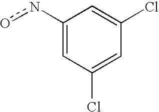 | C | 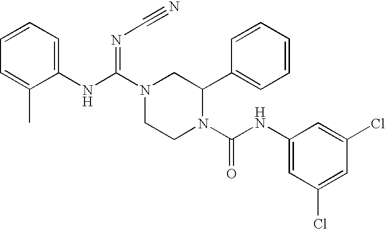 | 2.73 (k) | 505 (M − H)⁻ |
| 1.79 | 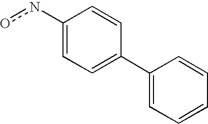 | C | 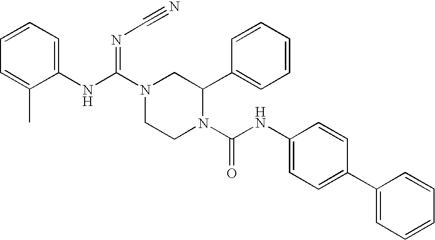 | 2.66 (k) | 513 (M − H)⁻ |
| 1.80 | 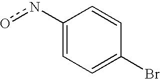 | C | 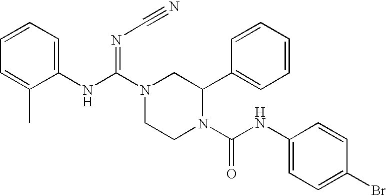 | 2.51 (k) | 515 (M − H)⁻ |
| 1.81 | 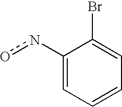 | C | 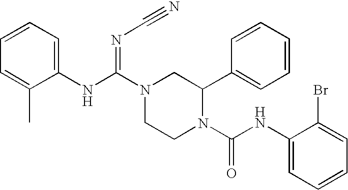 | 2.51 (k) | 515 (M − H)⁻ |
| 1.82 | 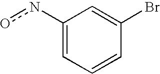 | C | 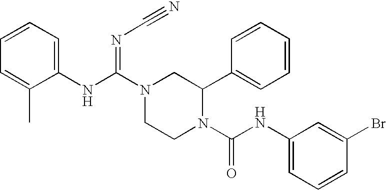 | 2.57 (k) | 515 (M − H)⁻ |
| 1.83 | 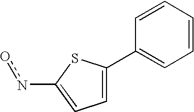 | C | 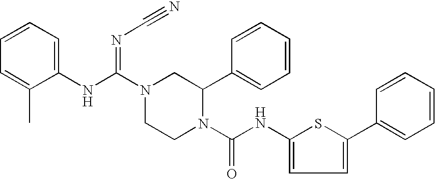 | 2.65 (k) | 519 (M − H)⁻ |
| 1.84 | 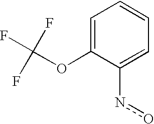 | C | 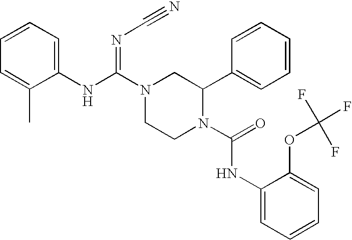 | 2.65 (k) | 521 (M − H)⁻ |
| 1.85 | 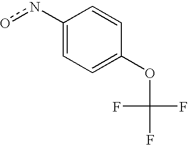 | C | 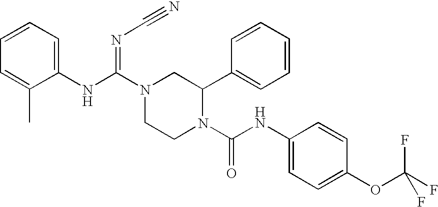 | 2.60 (k) | 521 (M − H)⁻ |
| 1.86 | 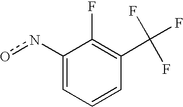 | C | 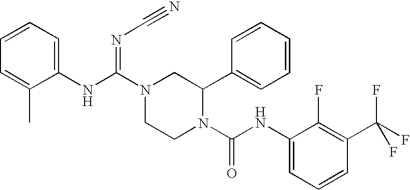 | 2.57 (k) | 523 (M − H)⁻ |
| 1.87 | 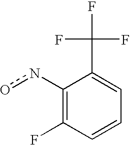 | C | 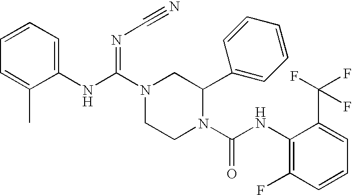 | 2.32 (k) | 523 (M − H)⁻ |
| 1.88 | 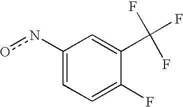 | C | 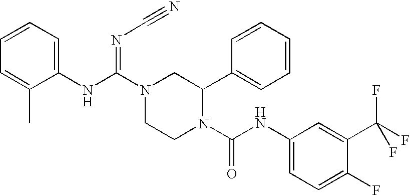 | 2.59 (k) | 523 (M − H)⁻ |
| 1.89 | 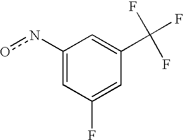 | C | 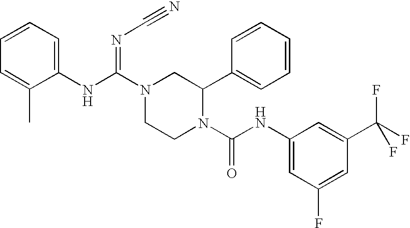 | 2.67 (k) | 523 (M − H)⁻ |
| 1.90 | 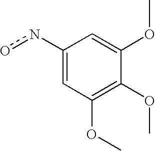 | C | 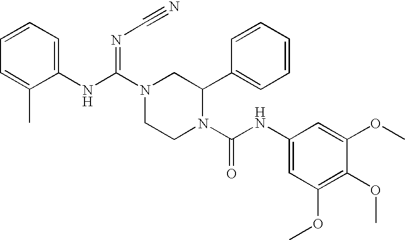 | 2.19 (k) | 527 (M − H)⁻ |
| 1.91 | 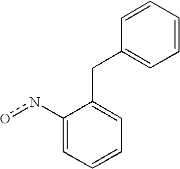 | C | 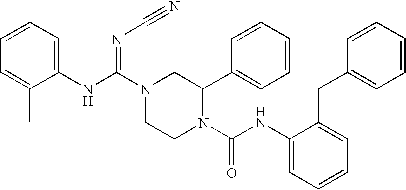 | 2.52 (k) | 527 (M − H)⁻ |
| 1.92 | 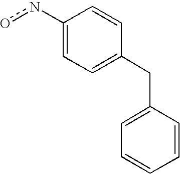 | C | 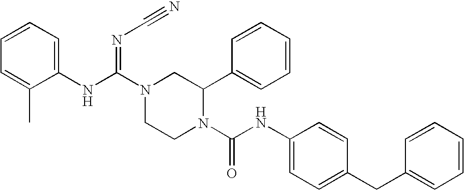 | 2.71 (k) | 527 (M − H)⁻ |
| 1.93 | 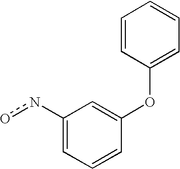 | C | 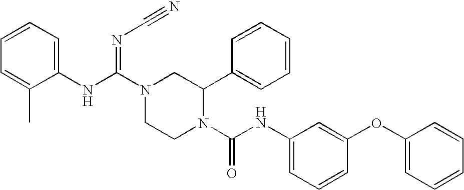 | 2.68 (k) | 529 (M − H)⁻ |
| 1.94 | 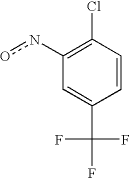 | C | 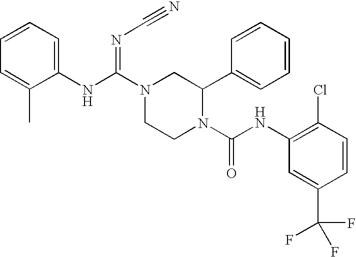 | 2.76 (k) | 539 (M − H)⁻ |
| 1.95 | 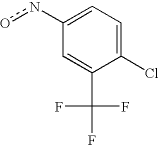 | C | 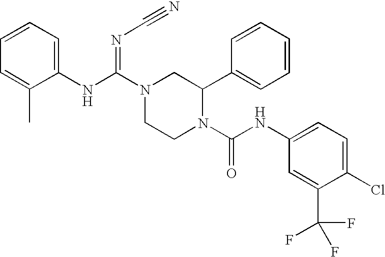 | 2.72 (k) | 539 (M − H)⁻ |
| 1.96 | 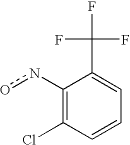 | C | 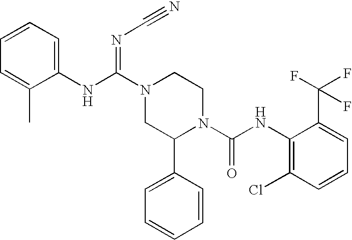 | 2.35 (k) | 539 (M − H)⁻ |
| 1.97 | 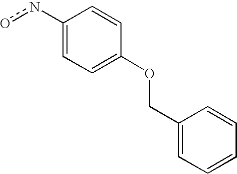 | C | 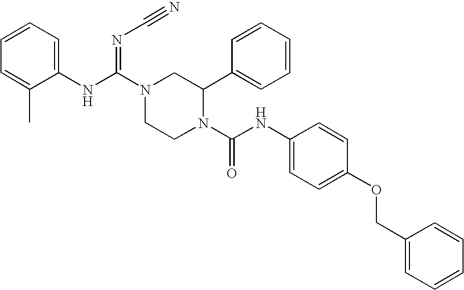 | 2.63 (k) | 543 (M − H)⁻ |
| 1.98 | 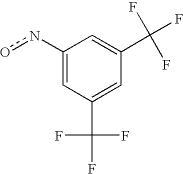 | C | 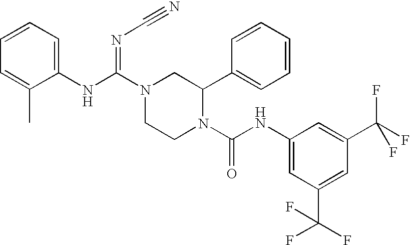 | 2.85 (k) | 573 (M − H)⁻ |
| 1.99 | 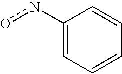 | C | 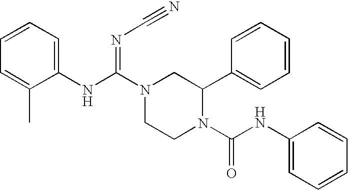 | 2.31 (k) | 437 (M − H)⁻ |
| 1.100 | 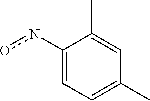 | C | 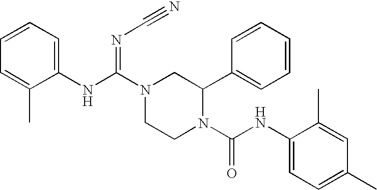 | 2.45 (k) | 465 (M − H)⁻ |
| 1.101 | 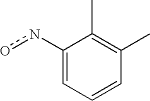 | C | 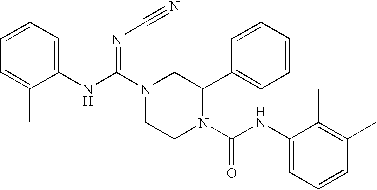 | 2.42 (k) | 465 (M − H)⁻ |
| 1.102 | 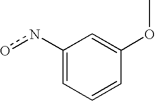 | C | 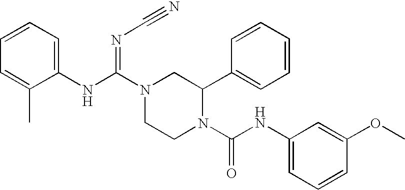 | 2.33 (k) | 467 (M − H)⁻ |
| 1.103 | 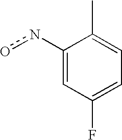 | C | 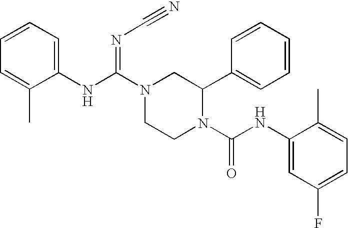 | 1.92 (d) | 469 (M − H)⁻ |
| 1.104 | 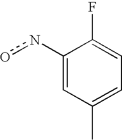 | C | 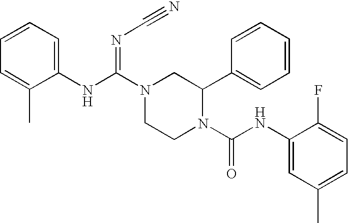 | 2.46 (k) | 469 (M − H)⁻ |
| 1.105 | 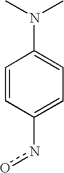 | C | 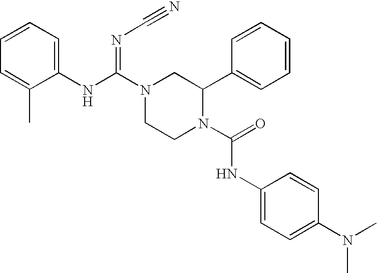 | 1.78 (d) | 480 (M − H)⁻ |
| 1.106 | 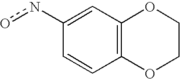 | C | 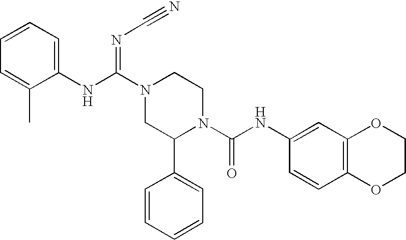 | 1.74 (d) | 495 (M − H)⁻ |
| 1.107 | 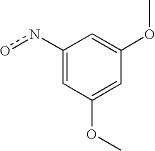 | C | 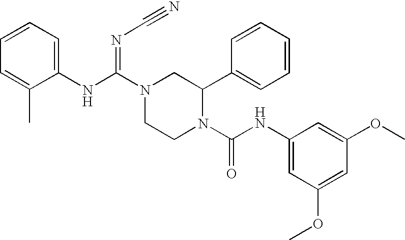 | 2.35 (k) | 497 (M − H)⁻ |
| 1.108 | 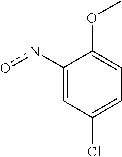 | C | 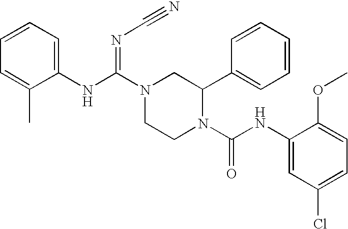 | 2.62 (k) | 501 (M − H)⁻ |
| 1.109 | 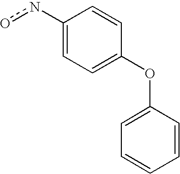 | C | 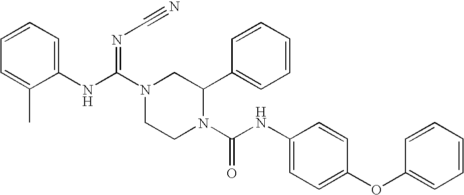 | 2.68 (k) | 529 (M − H)⁻ |
| 1.110 | 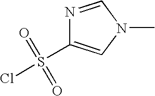 | E | 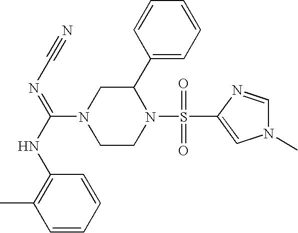 | 2.02 (k) | 462.5 (M − H)⁻ |
| 1.111 | 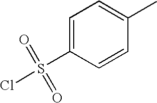 | E | 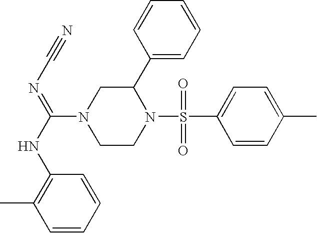 | 2.56 (k) | 472.6 (M − H)⁻ |
| 1.112 | 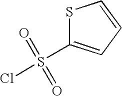 | E | 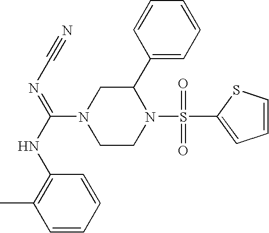 | 2.43 (k) | 464.6 (M − H)⁻ |
| 1.113 | 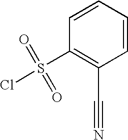 | E | 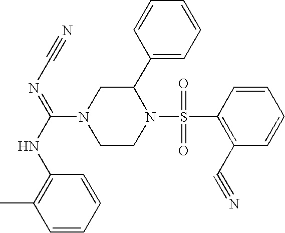 | 2.41 (k) | 483.5 (M − H)⁻ |
| 1.114 | 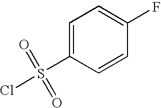 | E | 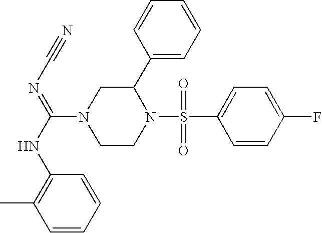 | 2.51 (k) | 476.5 (M − H)⁻ |
| 1.115 | 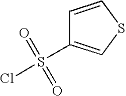 | E | 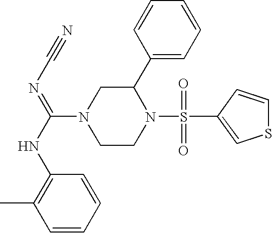 | 2.4 (k) | 464.6 (M − H)⁻ |
| 1.116 | 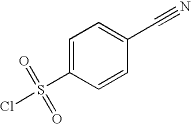 | E | 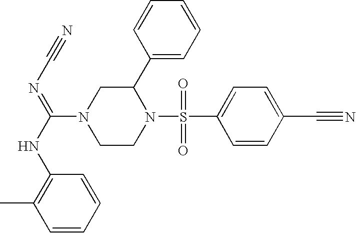 | 2.44 (k) | 483.5 (M − H)⁻ |
| 1.117 | 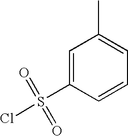 | E | 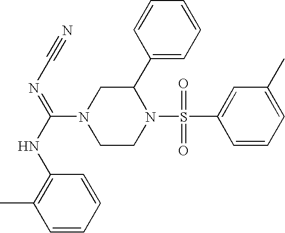 | 2.57 (k) | 472.6 (M − H)⁻ |
| 1.118 | 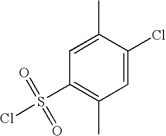 | E | 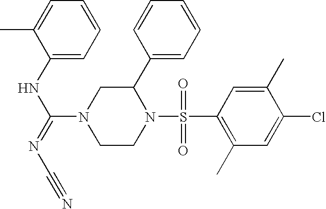 | 2.92 (k) | 521 (M − H)⁻ |
| 1.119 | 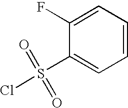 | E | 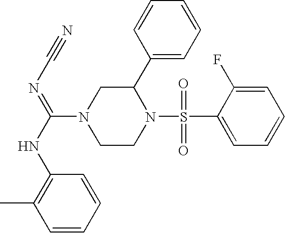 | 2.48 (k) | 476.5 (M − H)⁻ |
| 1.120 | 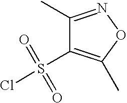 | E | 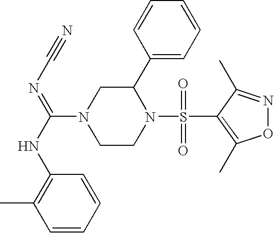 | 2.42 (k) | 477.5 (M − H)⁻ |
| 1.121 | 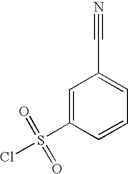 | E | 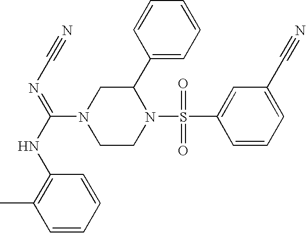 | 2.43 (k) | 483.5 (M − H)⁻ |
| 1.122 | 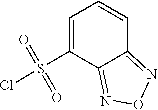 | E | 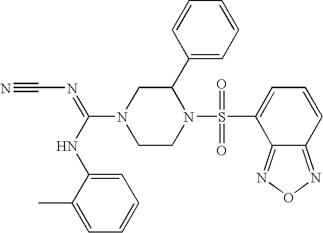 | 2.45 (k) | 500.5 (M − H)⁻ |
| 1.123 | 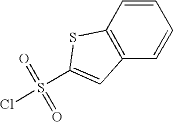 | E | 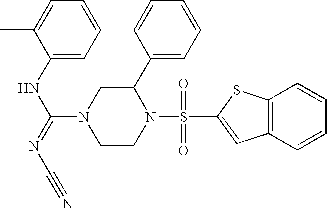 | 2.71 (k) | 514.6 (M − H)⁻ |
| 1.124 | 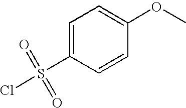 | E | 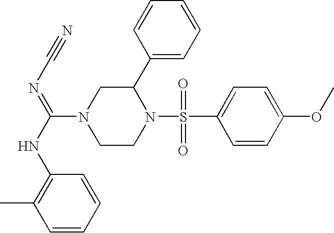 | 2.47 (k) | 488.6 (M − H)⁻ |
| 1.125 | 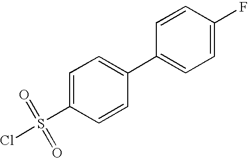 | E | 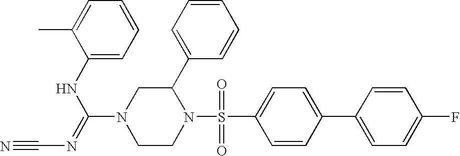 | 2.84 (k) | 552.6 (M − H)⁻ |
| 1.126 | 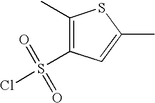 | E | 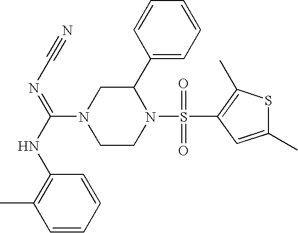 | 2.66 (k) | 492.6 (M − H)⁻ |
| 1.127 | 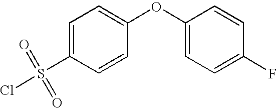 | E | 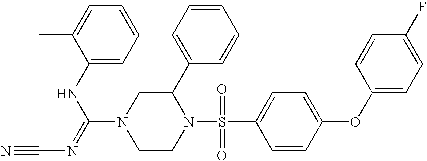 | 2.84 (k) | 568.6 (M − H)⁻ |
| 1.128 | 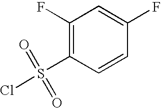 | E | 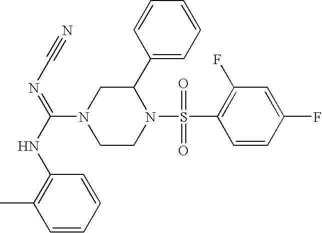 | 2.55 (k) | 494.5 (M − H)⁻ |
| 1.129 | 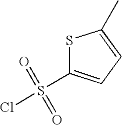 | E | 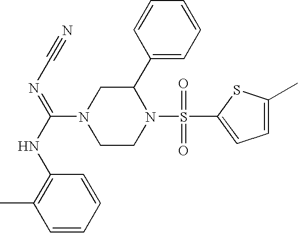 | 2.54 (k) | 478.6 (M − H)⁻ |
| 1.130 | 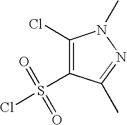 | E | 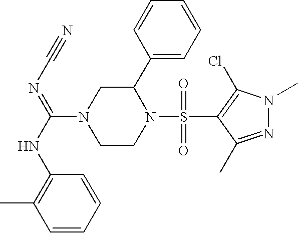 | 2.34 (k) | 511 (M − H)⁻ |
| 1.131 | 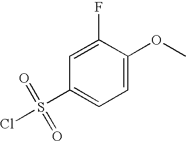 | E | 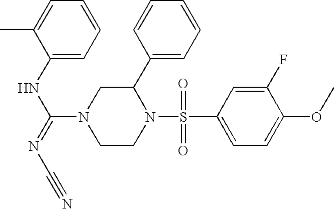 | 2.51 (k) | 506.5 (M − H)⁻ |
| 1.132 | 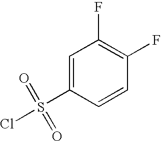 | E | 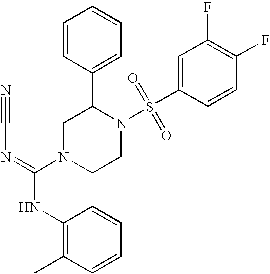 | 2.58 (k) | 494.5 (M − H)⁻ |
| 1.133 | 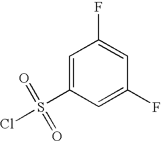 | E | 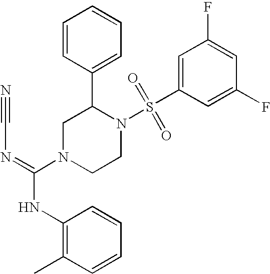 | 2.6 (k) | 494.5 (M − H)⁻ |
| 1.134 | 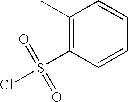 | E | 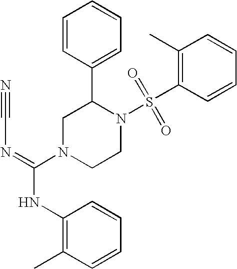 | 2.58 (k) | 472.6 (M − H)⁻ |
| 1.135 | 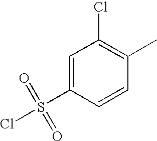 | E | 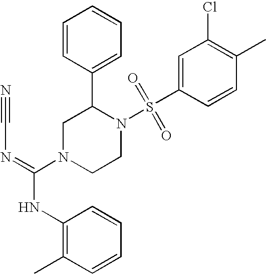 | 2.74 (k) | 507 (M − H)⁻ |
| 1.136 | 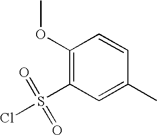 | E | 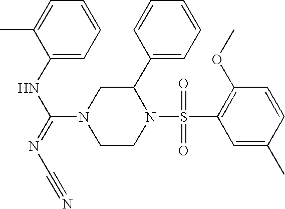 | 2.56 (k) | 502.6 (M − H)⁻ |
| 1.137 | 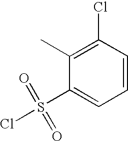 | E | 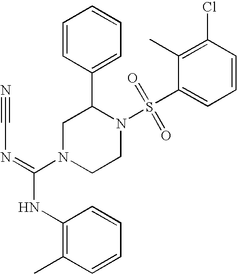 | 2.78 (k) | 507 (M − H)⁻ |
| 1.138 | 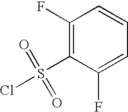 | E | 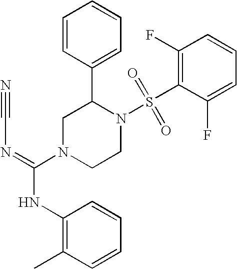 | 2.47 (k) | 494.5 (M − H)⁻ |
| 1.139 | 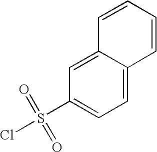 | E | 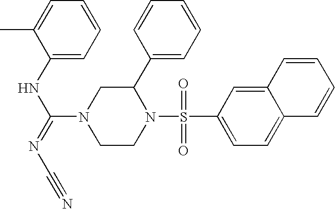 | 2.7 (k) | 508.6 (M − H)⁻ |
| 1.140 | 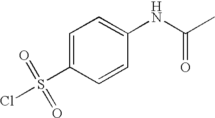 | E | 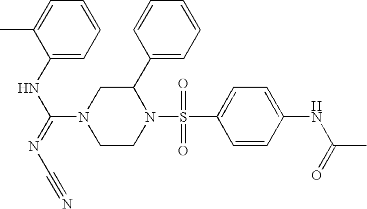 | 2.2 (k) | 515.6 (M − H)⁻ |
| 1.141 | 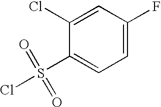 | E | 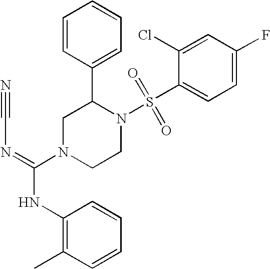 | 2.62 (k) | 511 (M − H)⁻ |
| 1.142 | 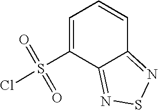 | E | 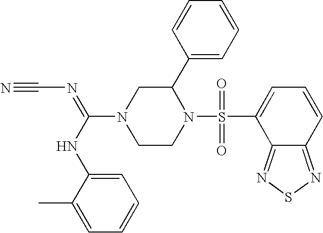 | 2.45 (k) | 516.6 (M − H)⁻ |
| 1.143 | 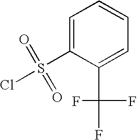 | E | 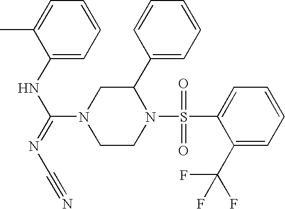 | 2.63 (k) | 526.5 (M − H)⁻ |
| 1.144 | 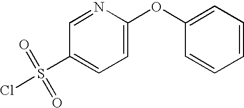 | E | 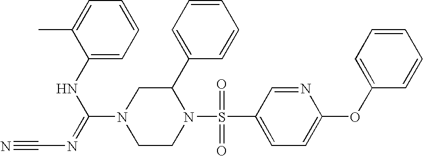 | 2.71 (k) | 551.6 (M − H)⁻ |
| 1.145 | 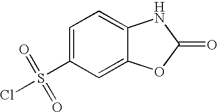 | E | 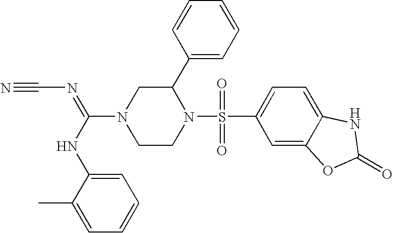 | 2.21 (k) | 515.5 (M − H)⁻ |
| 1.146 | 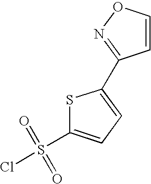 | E | 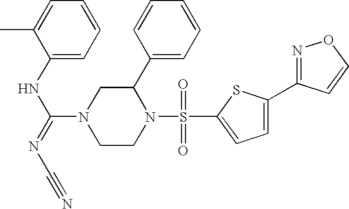 | 2.52 (k) | 531.6 (M − H)⁻ |
| 1.147 | 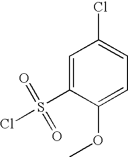 | E | 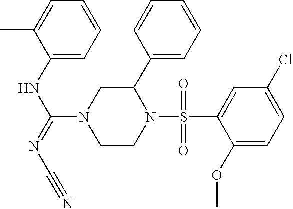 | 2.62 (k) | 523 (M − H)⁻ |
| 1.148 | 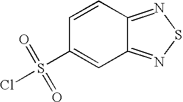 | E | 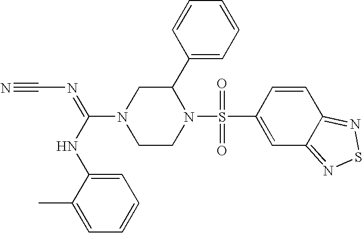 | 2.52 (k) | 516.6 (M − H)⁻ |
| 1.149 | 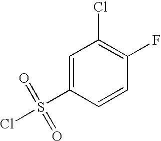 | E | 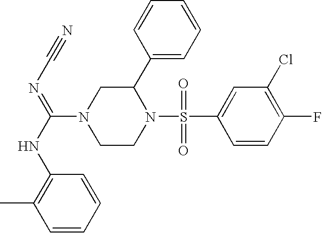 | 2.67 (k) | 511 (M − H)⁻ |
| 1.150 | 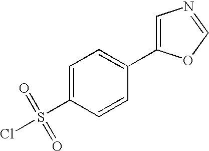 | E | 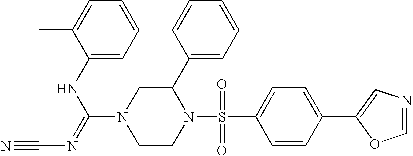 | 2.36 (k) | 525.6 (M − H)⁻ |
| 1.151 | 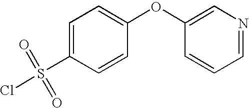 | E | 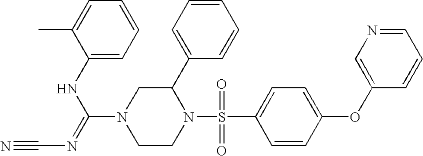 | 2.43 (k) | 551.6 (M − H)⁻ |
| 1.152 | 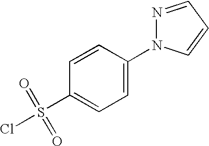 | E | 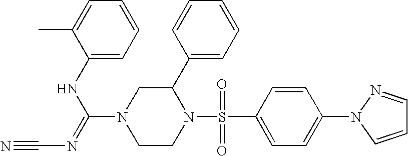 | 2.48 (k) | 524.6 (M − H)⁻ |
| 1.153 | 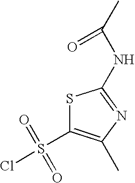 | E | 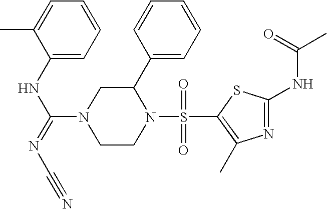 | 2.22 (k) | 536.6 (M − H)⁻ |
| 1.154 | 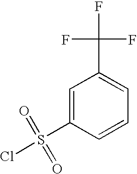 | E | 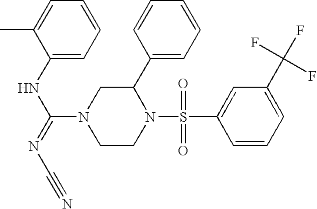 | 2.69 (k) | 526.5 (M − H)⁻ |
| 1.155 | 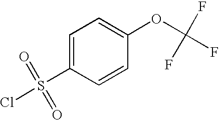 | E | 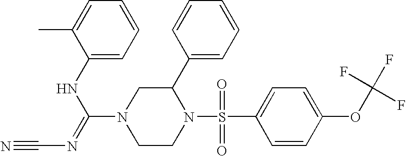 | 2.75 (k) | 542.5 (M − H)⁻ |
| 1.156 | 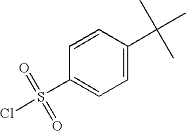 | E | 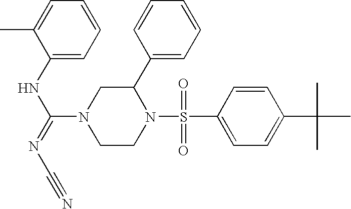 | 2.89 (k) | 514.6 (M − H)⁻ |
| 1.157 | 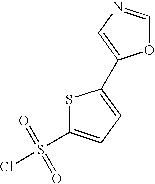 | E | 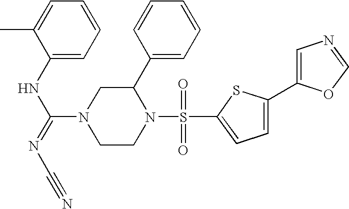 | 2.4 (k) | 531.6 (M − H)⁻ |
| 1.158 | 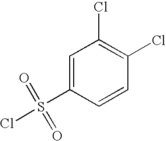 | E | 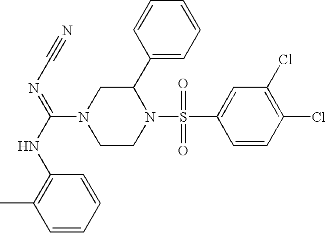 | 2.78 (k) | 527.4 (M − H)⁻ |
| 1.159 | 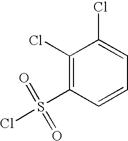 | E | 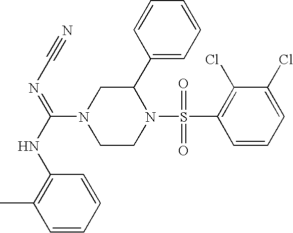 | 2.71 (k) | 527.4 (M − H)⁻ |
| 1.160 | 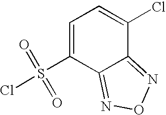 | E | 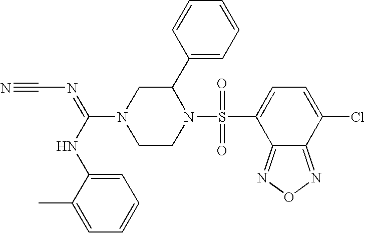 | 2.63 (k) | 535 (M − H)⁻ |
| 1.161 | 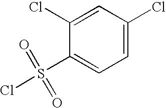 | E | 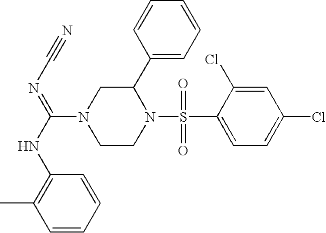 | 2.77 (k) | 527.4 (M − H)⁻ |
| 1.162 | 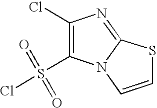 | E | 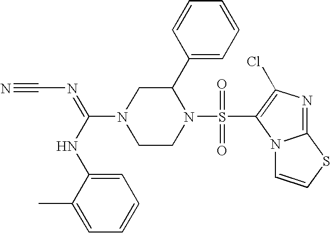 | 2.4 (k) | 539 (M − H)⁻ |
| 1.163 | 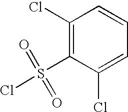 | E | 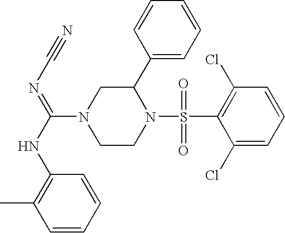 | 2.63 (k) | 527.4 (M − H)⁻ |
| 1.164 | 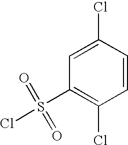 | E | 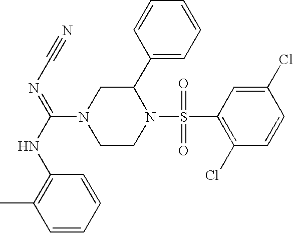 | 2.74 (k) | 527.4 (M − H)⁻ |
| 1.165 | 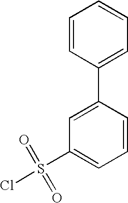 | E | 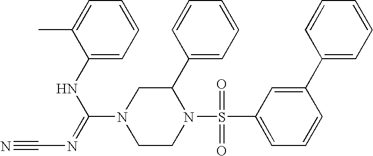 | 2.81 (k) | 534.6 (M − H)⁻ |
| 1.166 | 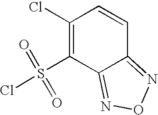 | E | 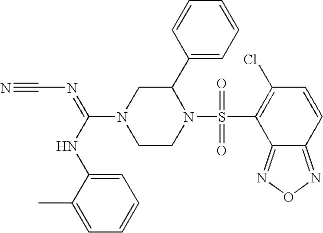 | 2.51 | 535 (M − H)⁻ |
| 1.167 | 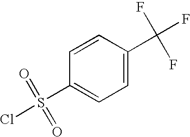 | E | 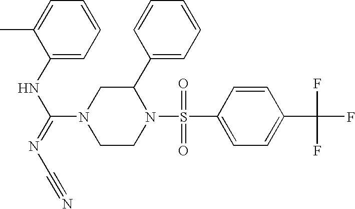 | 2.71 (k) | 526.5 (M − H)⁻ |
| 1.168 | 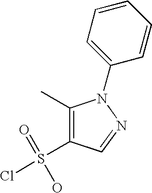 | E | 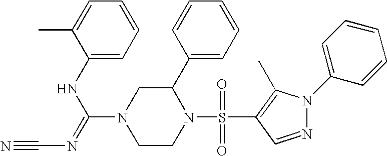 | 2.54 (k) | 538.6 (M − H)⁻ |
| 1.169 | 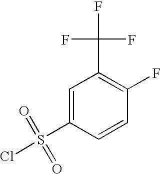 | E | 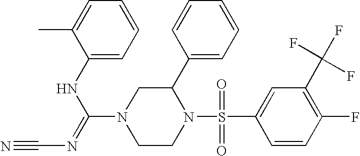 | 2.74 (k) | 544.5 (M − H)⁻ |
| 1.170 | 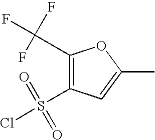 | E | 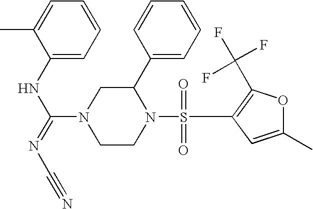 | 2.74 (k) | 530.5 (M − H)⁻ |
| 1.171 | 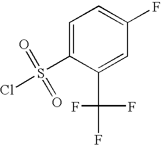 | E | 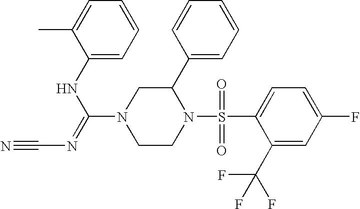 | 2.7 (k) | 544.5 (M − H)⁻ |
| 1.172 | 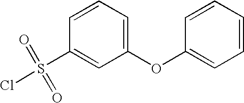 | E | 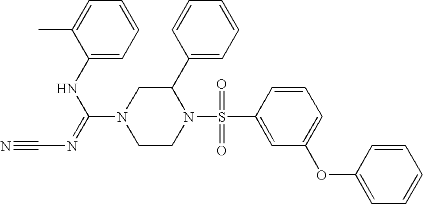 | 2.78 (k) | 550.6 (M − H)⁻ |
| 1.173 | 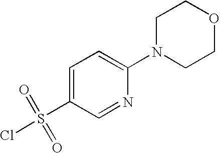 | E | 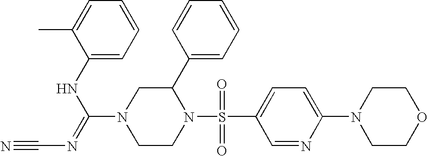 | 2.33 (k) | 544.6 (M − H)⁻ |
| 1.174 | 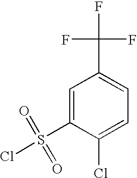 | E | 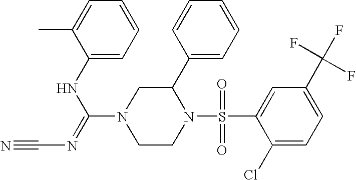 | 2.79 (k) | 561 (M − H)⁻ |
| 1.175 | 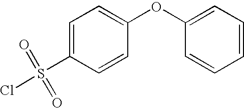 | E | 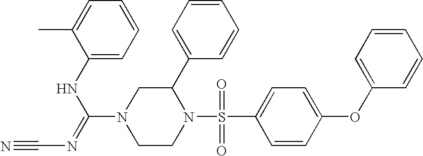 | 2.83 (k) | 550.6 (M − H)⁻ |
| 1.176 | 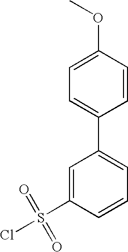 | E | 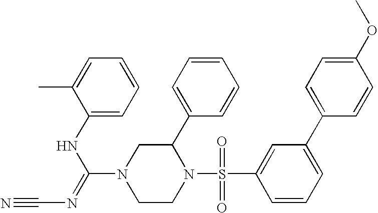 | 2.81 (k) | 564.6 (M − H)⁻ |
| 1.177 | 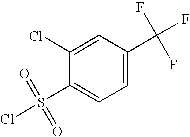 | E | 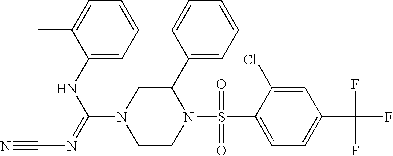 | 2.82 (k) | 561 (M − H)⁻ |
| 1.178 | 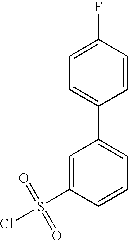 | E | 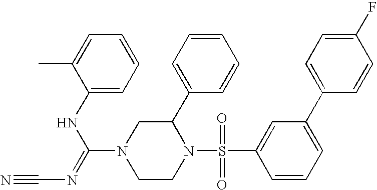 | 2.83 (k) | 552.6 (M − H)⁻ |
| 1.179 | 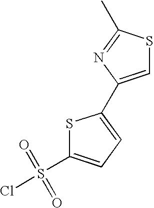 | E | 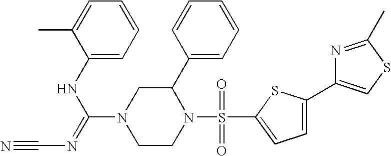 | 2.63 (k) | 561.7 (M − H)⁻ |
| 1.180 | 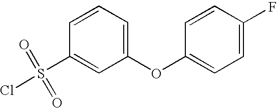 | E | 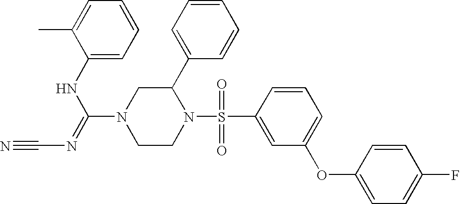 | 2.84 (k) | 568.6 (M − H)⁻ |
| 1.181 | 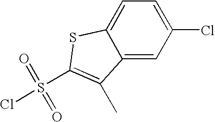 | E | 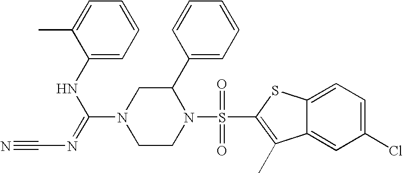 | 2.97 (k) | 563.1 (M − H)⁻ |
| 1.182 | 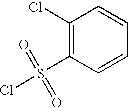 | E | 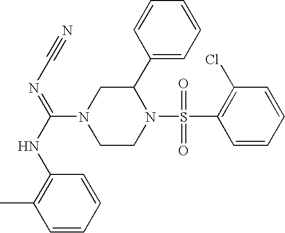 | 2.56 (k) | 493 (M − H)⁻ |
| 1.183 | 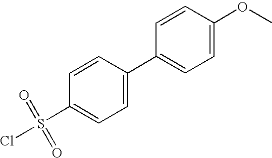 | E | 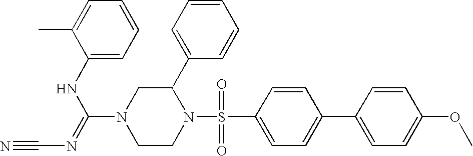 | 2.8 (k) | 564.6 (M − H)⁻ |
| 1.184 | 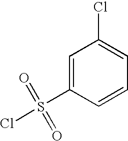 | E | 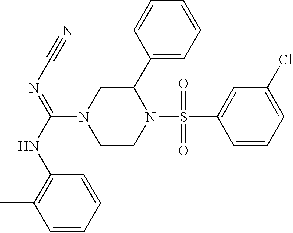 | 2.63 (k) | 493 (M − H)⁻ |
| 1.185 | 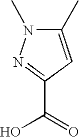 | D | 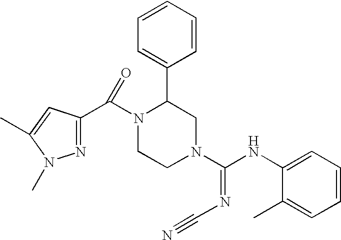 | 2.06 (k) | 443 (M + H)⁺ |
| 1.186 | 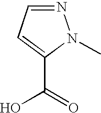 | D | 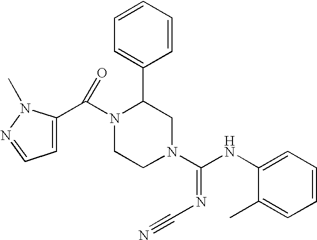 | 1.96 (k) | 429 (M + H)⁺ |
| 1.187 | 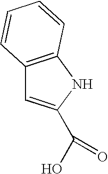 | D | 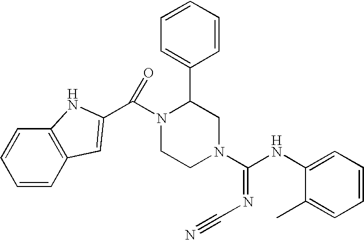 | 2.45 (k) | 464 (M + H)⁺ |
| 1.188 | 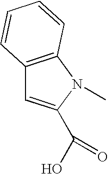 | D | 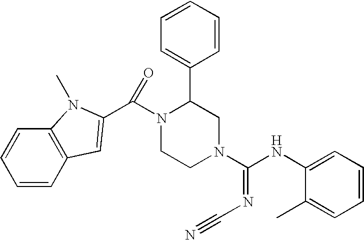 | 2.57 (k) | 478 (M + H)⁺ |
| 1.189 | 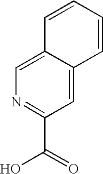 | D | 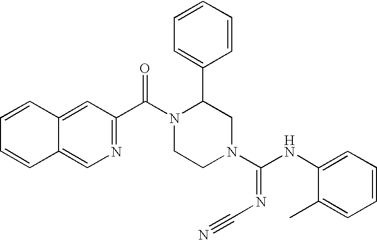 | 2.31 (k) | 476 (M + H)⁺ |
| 1.190 | 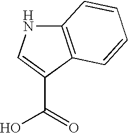 | D | 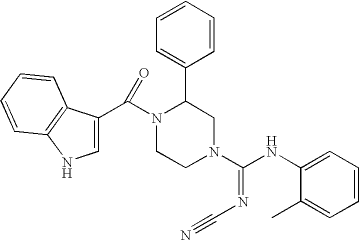 | 2.18 (k) | 464 (M + H)⁺ |
| 1.191 | 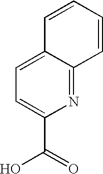 | D | 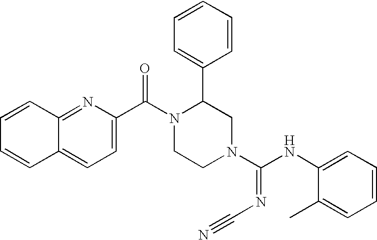 | 2.35 (k) | 476 (M + H)⁺ |
| 1.192 | 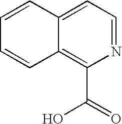 | D | 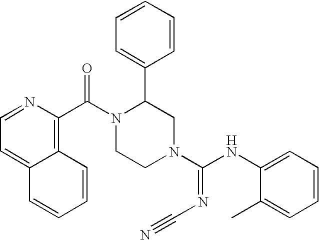 | 2.28 (k) | 476 (M + H)⁺ |
| 1.193 | 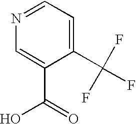 | D | 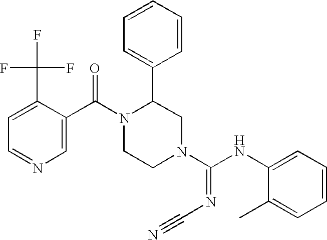 | 2.16 (k) | 494 (M + H)⁺ |
| 1.194 | 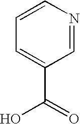 | D | 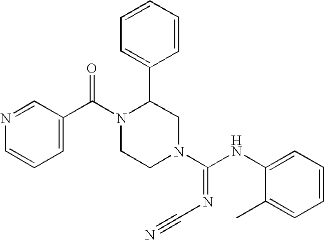 | 1.88 (k) | 426 (M + H)⁺ |
| 1.195 | 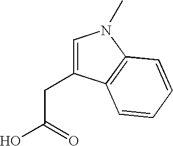 | D | 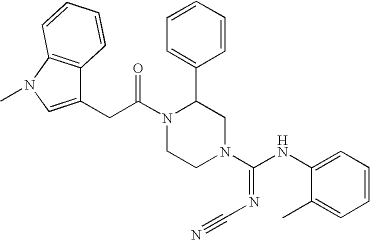 | 2.42 (k) | 492 (M + H)⁺ |
| 1.196 | 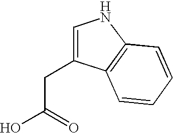 | D | 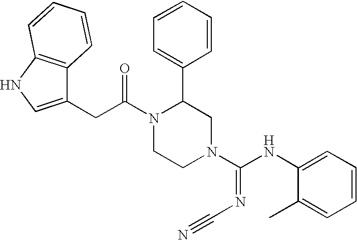 | 2.14 (k) | 478 (M + H)⁺ |
| 1.197 | 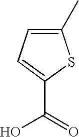 | D | 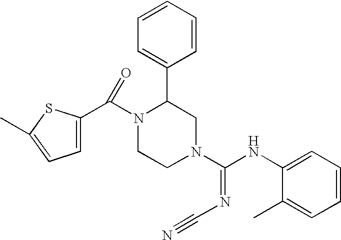 | 2.39 (k) | 445 (M + H)⁺ |
| 1.198 | 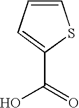 | D | 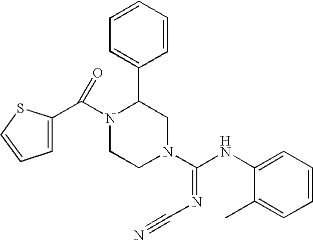 | 2.25 (k) | 431 (M + H)⁺ |
| 1.199 | 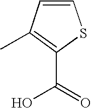 | D | 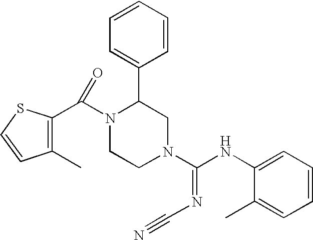 | 2.32 (k) | 445 (M + H)⁺ |
| 1.200 | 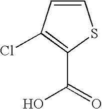 | D | 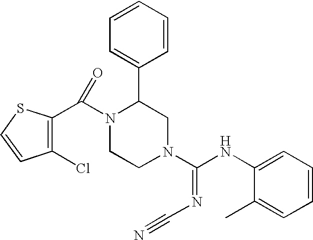 | 2.32 (k) | 465 (M + H)⁺ |
| 1.201 | 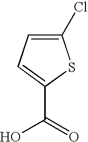 | D | 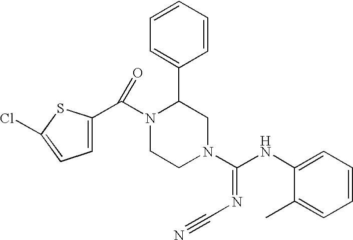 | 2.5 (k) | 465 (M + H)⁺ |
| 1.202 | 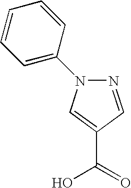 | D | 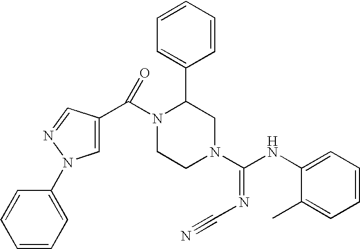 | 2.33 (k) | 491 (M + H)⁺ |
| 1.203 | 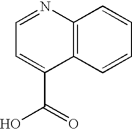 | D | 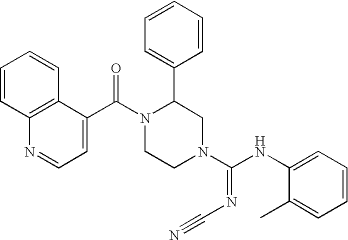 | 2.09 (k) | 476 (M + H)⁺ |
| 1.204 | 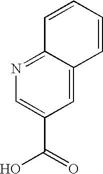 | D | 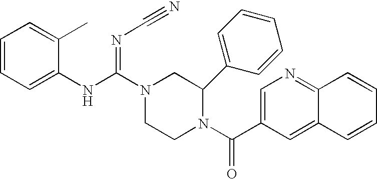 | 2.18 (k) | 476 (M + H)⁺ |
| 1.205 | 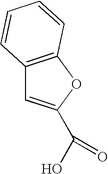 | D | 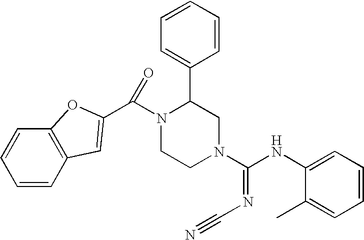 | 2.45 (k) | 465 (M + H)⁺ |
| 1.206 | 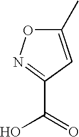 | D | 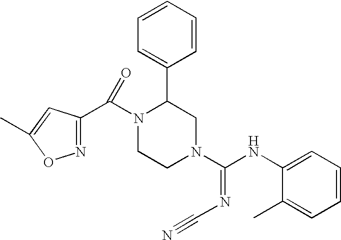 | 2.21 (k) | 430 (M + H)⁺ |
| 1.207 | 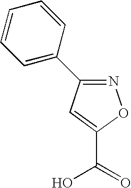 | D | 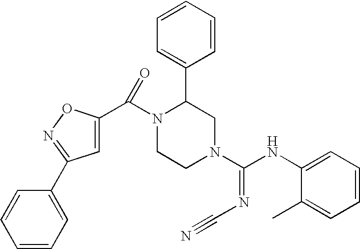 | 2.55 (k) | 492 (M + H)⁺ |
| 1.208 | 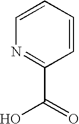 | D | 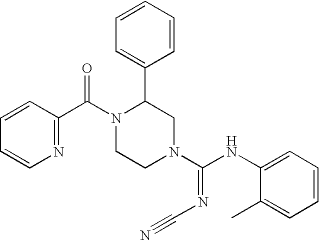 | 2 (k) | 426 (M + H)⁺ |
| 1.209 | 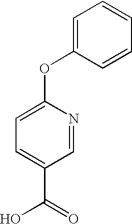 | D | 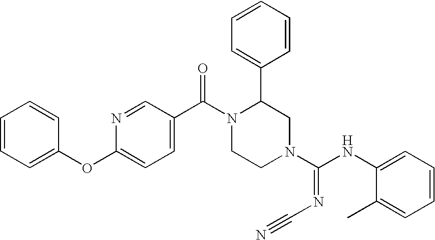 | 2.48 (k) | 518 (M + H)⁺ |
| 1.210 | 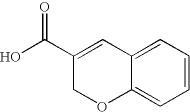 | D | 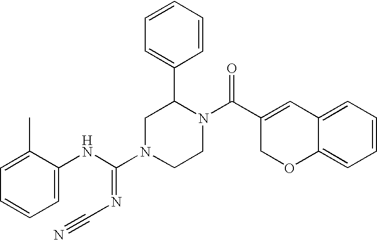 | 2.49 (k) | 479 (M + H)⁺ |
| 1.211 | 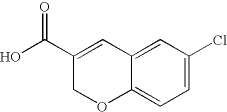 | D | 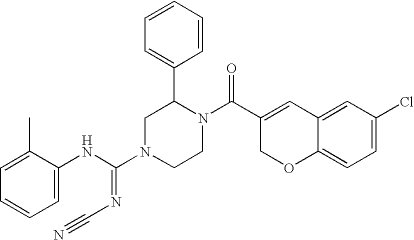 | 2.68 (k) | 513 (M + H)⁺ |
| 1.212 | 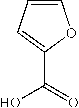 | D | 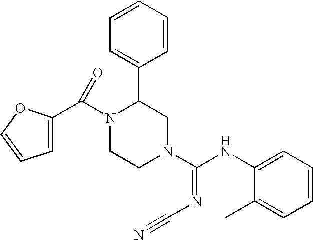 | 2.1 (k) | 415 (M + H)⁺ |
| 1.213 | 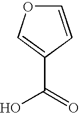 | D | 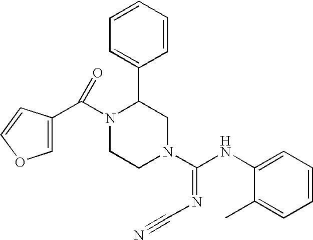 | 2.09 (k) | 415 (M + H)⁺ |
| 1.214 | 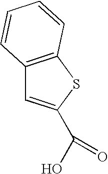 | D | 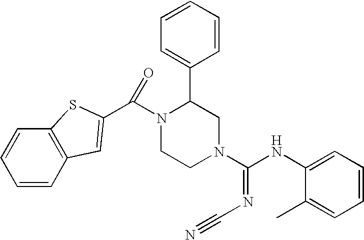 | 2.54 (k) | 481 (M + H)⁺ |
| 1.215 | 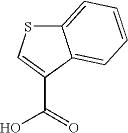 | D | 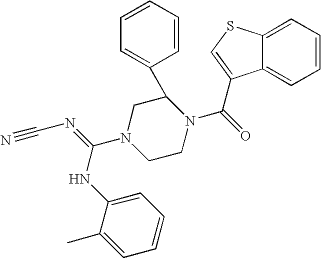 | 2.46 (k) | 481 (M + H)⁺ |
| 1.216 | 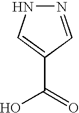 | D | 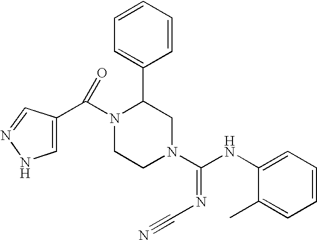 | 1.73 (k) | 415 (M + H)⁺ |
| 1.217 | 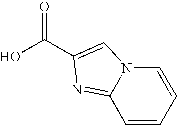 | D | 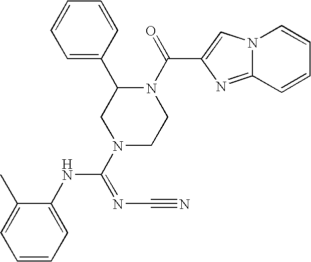 | 2.02 (k) | 465 (M + H)⁺ |
| 1.218 | 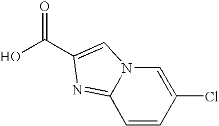 | D | 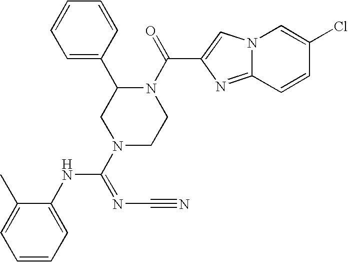 | 2.26 (k) | 499 (M + H)⁺ |
| 1.219 | 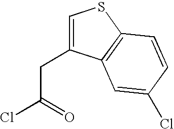 | D | 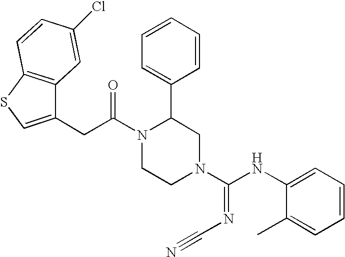 | 2.68 (k) | 529 (M + H)⁺ |
| 1.220 | 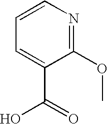 | D | 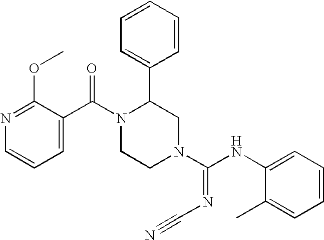 | 2.1 (k) | 456 (M + H)⁺ |
| 1.221 | 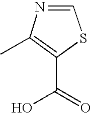 | D | 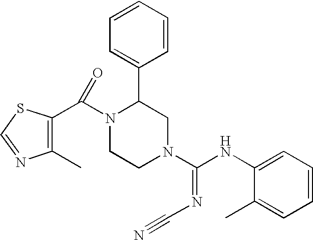 | 1.97 (k) | 446 (M + H)⁺ |
| 1.222 | 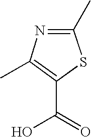 | D | 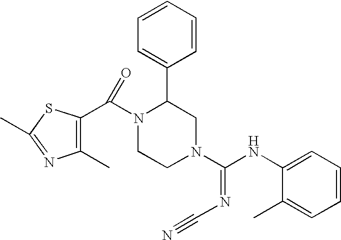 | 2.01 (k) | 460 (M + H)⁺ |
| 1.223 | 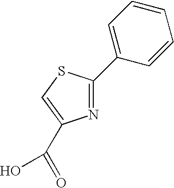 | D | 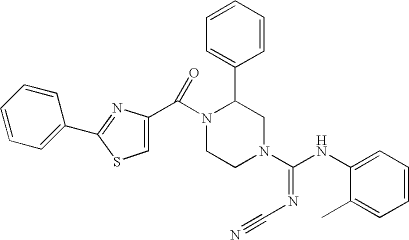 | 2.58 (k) | 508 (M + H)⁺ |
| 1.224 | 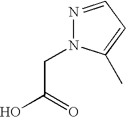 | D | 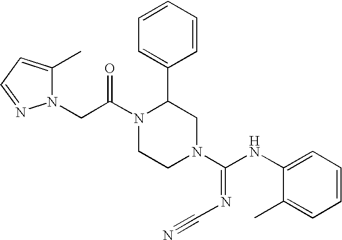 | 1.98 (k) | 443 (M + H)⁺ |
| 1.225 | 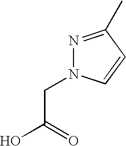 | D | 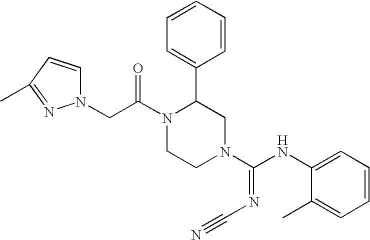 | 1.99 (k) | 443 (M + H)⁺ |
| 1.226 | 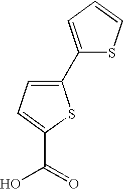 | D | 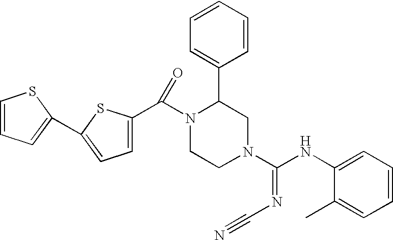 | 2.65 (k) | 513 (M + H)⁺ |
| 1.227 | 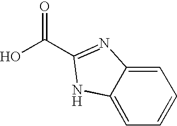 | D | 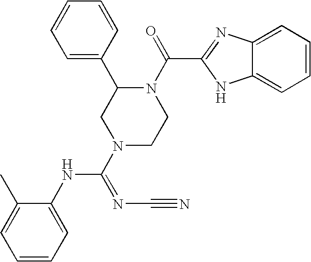 | 2.14 (k) | 465 (M + H)⁺ |
| 1.228 | 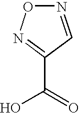 | D | 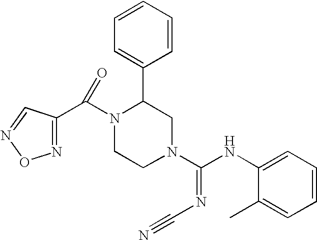 | 2.06 (k) | 417 (M + H)⁺ |
| 1.229 | 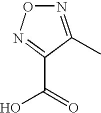 | D | 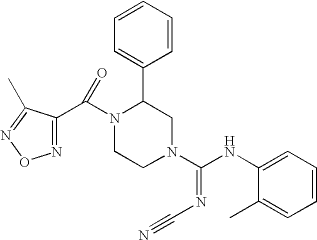 | 2.32 (k) | 431 (M + H)⁺ |
| 1.230 | 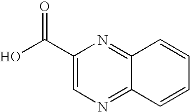 | D | 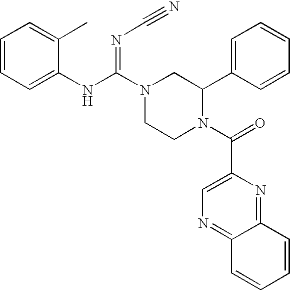 | 2.3 (k) | 477 (M + H)⁺ |
| 1.231 | 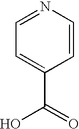 | D | 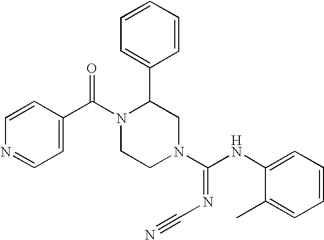 | 1.88 (k) | 426 (M + H)⁺ |
| 1.232 | 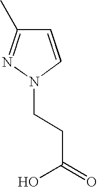 | D | 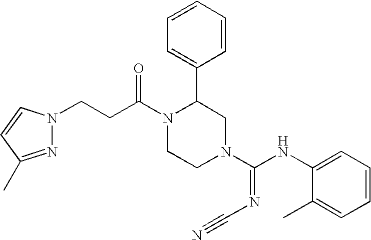 | 2.02 (k) | 457 (M + H)⁺ |
| 1.233 | 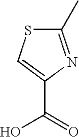 | D | 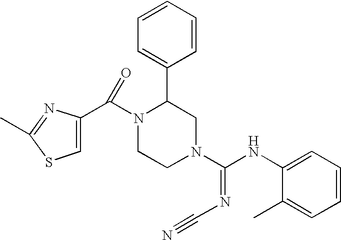 | 2.09 (k) | 446 (M + H)⁺ |
| 1.234 | 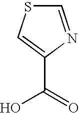 | D | 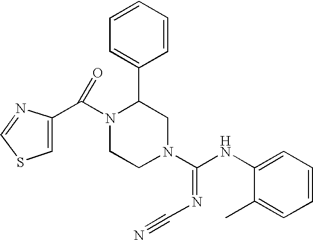 | 2 (k) | 432 (M + H)⁺ |
| 1.235 | 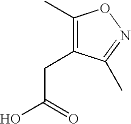 | D | 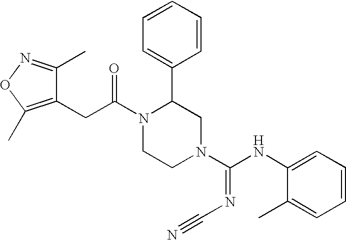 | 2.04 (k) | 458 (M + H)⁺ |
| 1.236 | 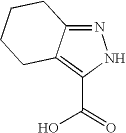 | D | 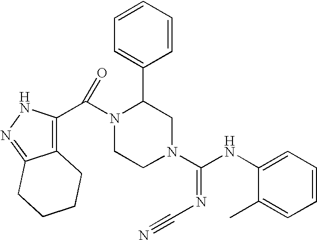 | 2.14 (k) | 469 (M + H)⁺ |
| 1.237 | 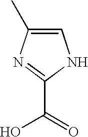 | D | 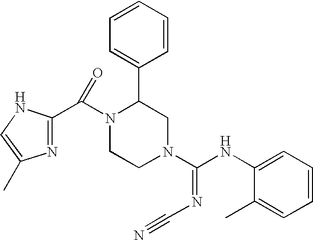 | 2.04 (k) | 429 (M + H)⁺ |
| 1.238 | 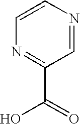 | D | 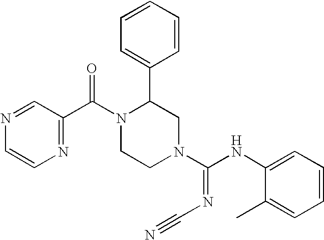 | 1.93 (k) | 427 (M + H)⁺ |
| 1.239 | 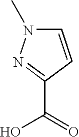 | D | 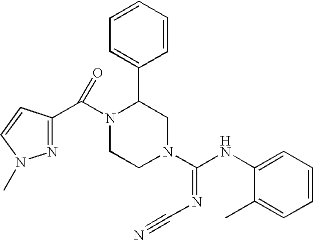 | 1.96 (k) | 429 (M + H)⁺ |
| 1.240 | 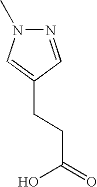 | D | 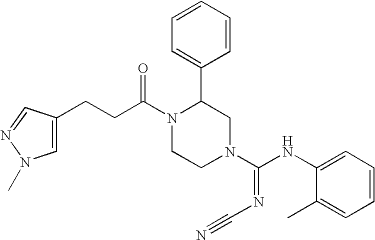 | 1.88 (k) | 457 (M + H)⁺ |
| 1.241 | 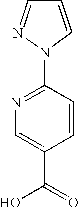 | D | 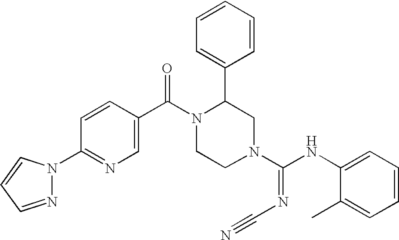 | 2.26 (k) | 492 (M + H)⁺ |
| 1.242 | 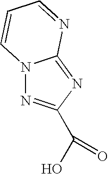 | D | 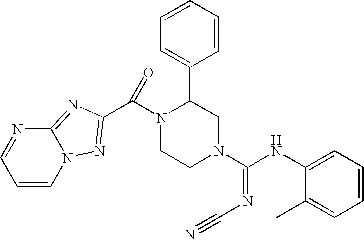 | 1.81 (k) | 467 (M + H)⁺ |
| 1.243 | 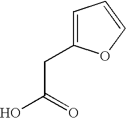 | D | 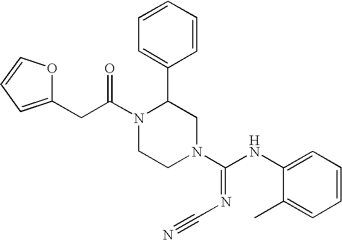 | 2.15 (k) | 429 (M + H)⁺ |
| 1.244 | 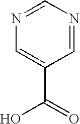 | D | 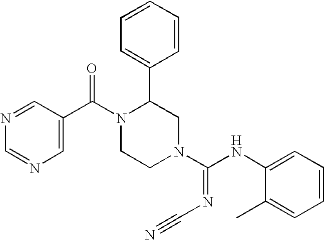 | 1.81 (k) | 427 (M + H)⁺ |
| 1.245 | 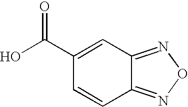 | D | 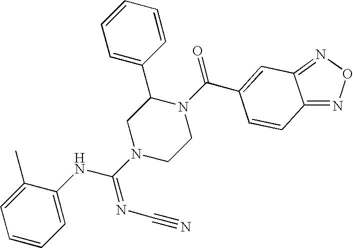 | 2.31 (k) | 467 (M + H)⁺ |
| 1.246 | 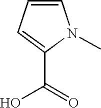 | D | 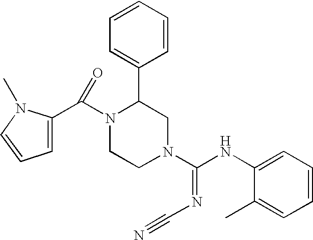 | 2.22 (k) | 428 (M + H)⁺ |
| 1.247 | 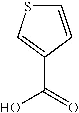 | D | 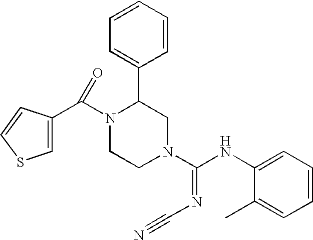 | 2.18 (k) | 431 (M + H)⁺ |
| 1.248 | 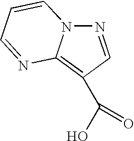 | D | 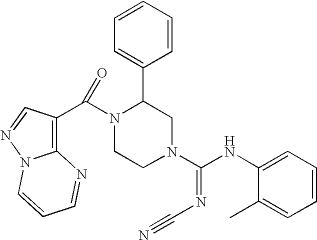 | 1.86 (k) | 466 (M + H)⁺ |
| 1.249 | 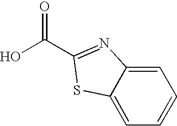 | D | 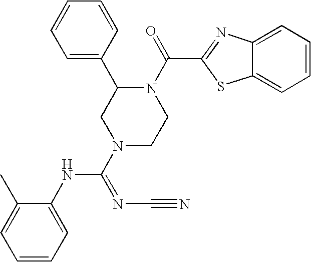 | 2.62 (k) | 482 (M + H)⁺ |
| 1.250 | 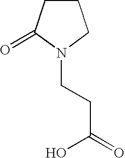 | D | 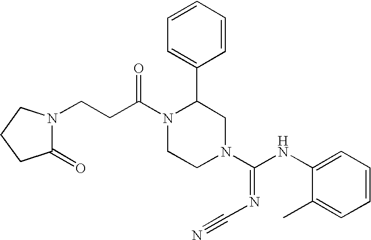 | 1.77 (k) | 460 (M + H)⁺ |
| 1.251 | 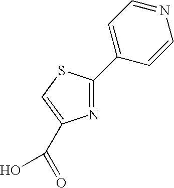 | D | 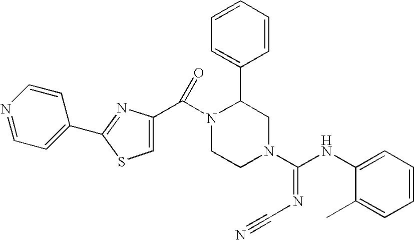 | 2.11 (k) | 509 (M + H)⁺ |
| 1.252 | 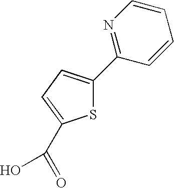 | D | 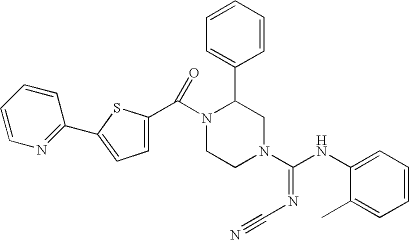 | 2.36 (k) | 508 (M + H)⁺ |
| 1.253 | 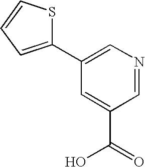 | D | 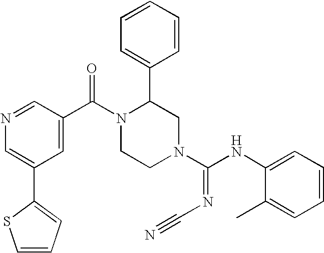 | 2.3 (k) | 508 (M + H)⁺ |
| 1.254 | 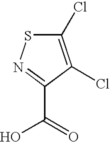 | D | 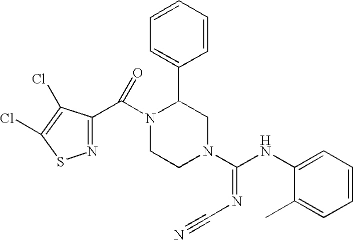 | 2.49 (k) | 500 (M + H)⁺ |
| 1.255 | 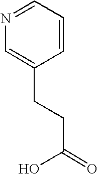 | D | 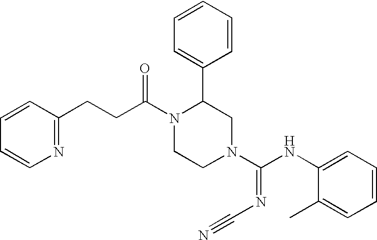 | 1.96 (k) | 454 (M + H)⁺ |
| 1.256 | 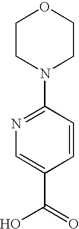 | D | 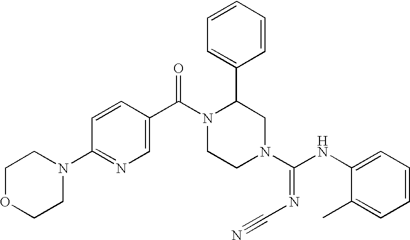 | 2.06 (k) | 511 (M + H)⁺ |
| 1.257 | 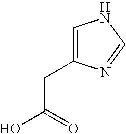 | D | 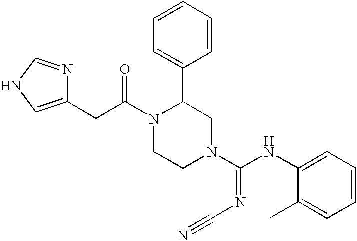 | 1.94 (k) | 429 (M + H)⁺ |
| 1.258 | 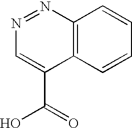 | D | 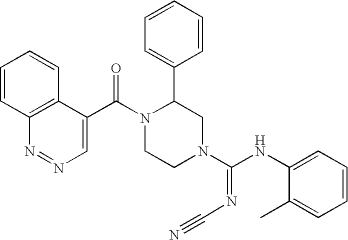 | 2 (k) | 477 (M + H)⁺ |
| 1.259 | 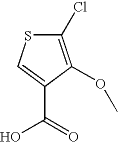 | D | 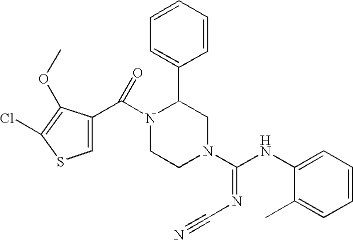 | 2.45 (k) | 495 (M + H)⁺ |
| 1.260 | 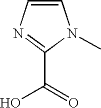 | D | 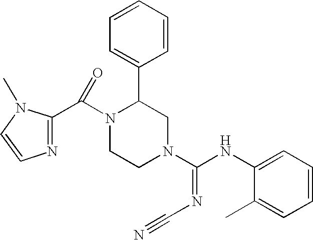 | 1.95 (k) | 429 (M + H)⁺ |
| 1.261 | 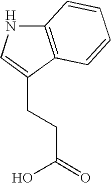 | D | 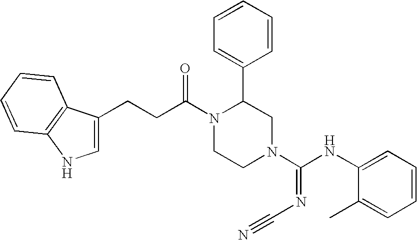 | 2.32 (k) | 492 (M + H)⁺ |
| 1.262 | 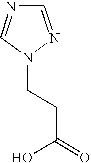 | D | 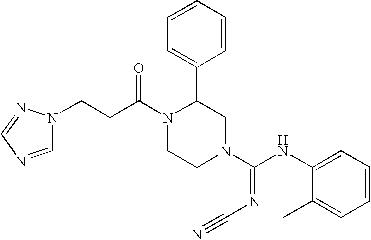 | 1.74 (k) | 444 (M + H)⁺ |
| 1.263 | 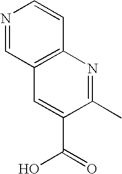 | D | 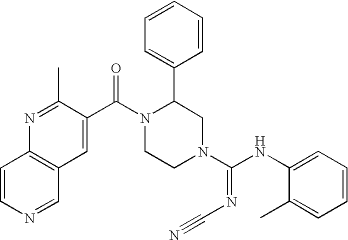 | 1.89 (k) | 491 (M + H)⁺ |
| 1.264 | 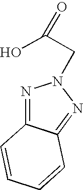 | D | 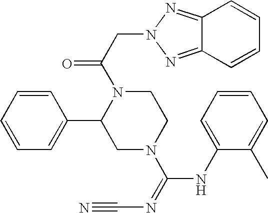 | 2.25 (k) | 480 (M + H)⁺ |
| 1.265 | 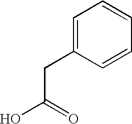 | D | 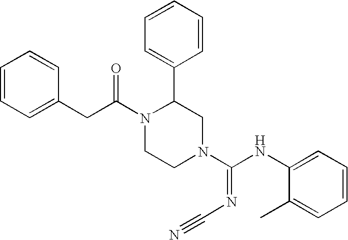 | 2.31 (k) | 439 (M + H)⁺ |
| 1.266 | 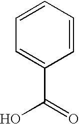 | D | 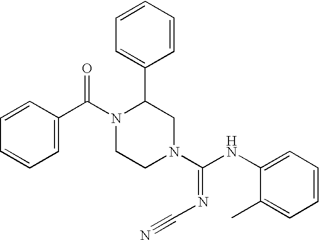 | 2.23 (k) | 425 (M + H)⁺ |
| 1.267 | 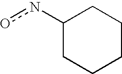 | C | 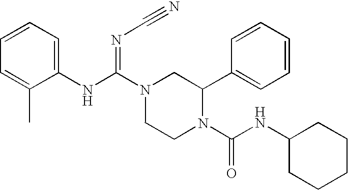 | 1.87 (d) | 443 (M − H)⁻ |
| 1.268 | 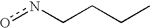 | C | 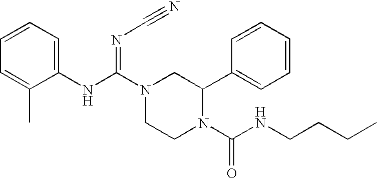 | 1.78 (d) | 417 (M − H)⁻ |
| 1.269 | 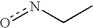 | C | 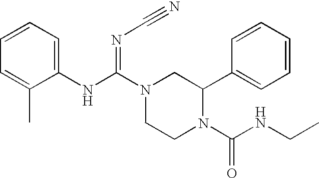 | 1.42 (d) | 389 (M − H)⁻ |
| 1.270 | 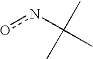 | C | 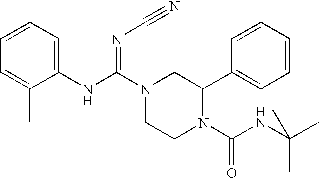 | 1.81 (d) | 417 (M − H)⁻ |
| 1.271 | 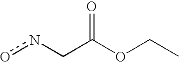 | C | 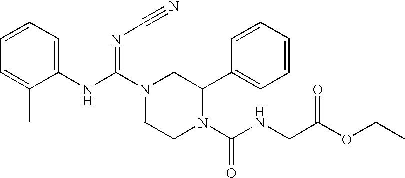 | 1.48 (d) | 447 (M − H)⁻ |
| 1.272 | 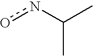 | C | 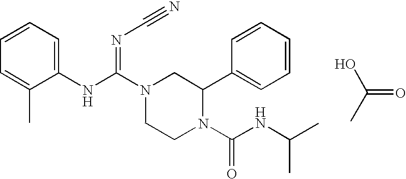 | 1.60 (d) | 403 (M − H)⁻ |
| 1.273 | 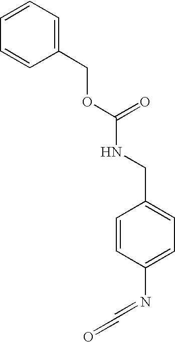 | C | 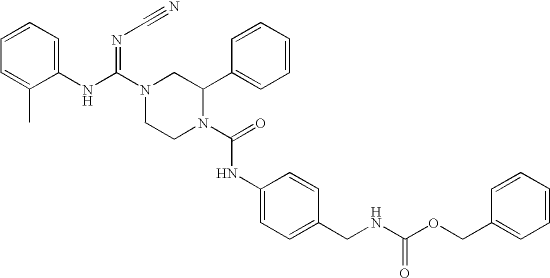 | 1.91 (d) | 600 (M − H)⁻ |
| 1.274 | 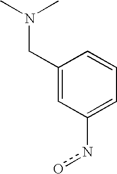 | C | 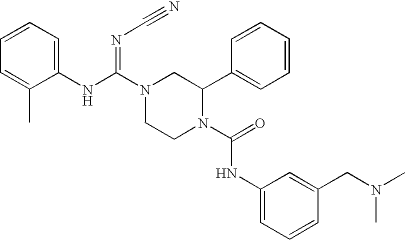 | 1.56 (d) | 494 (M − H)⁻ |
| 1.275 | 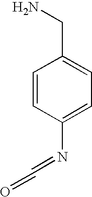 | C | 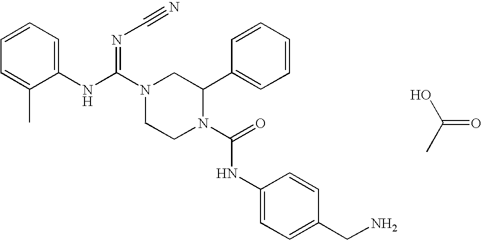 | 1.39 (d) | 466 (M − H)⁻ |
| 1.276 | 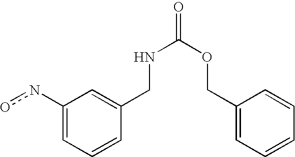 | C | 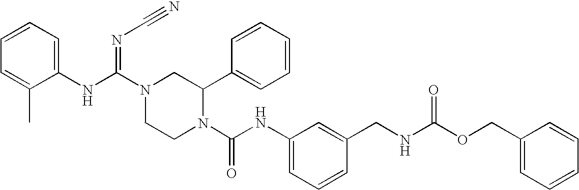 | 2.00 (d) | 600 (M − H)⁻ |
| 1.278 | 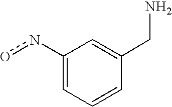 | C | 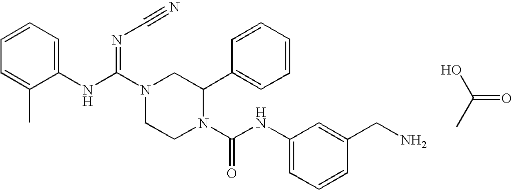 | 1.14 (d) | 466 (M − H)⁻ |
| 1.279 | 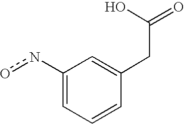 | C | 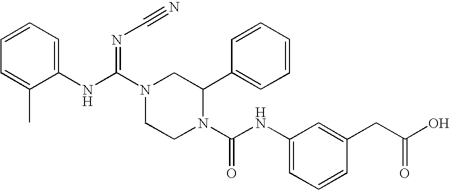 | 0.93 (d) | 495 (M − H)⁻ |
| 1.280 | 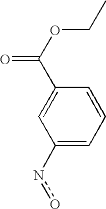 | C | 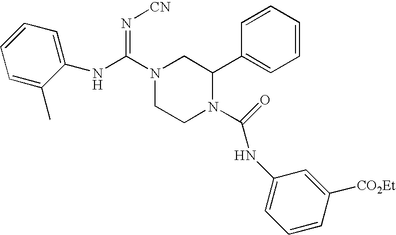 | 1.72 (d) | 511 (M + H)⁺ |
| 1.281 | 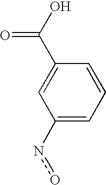 | C | 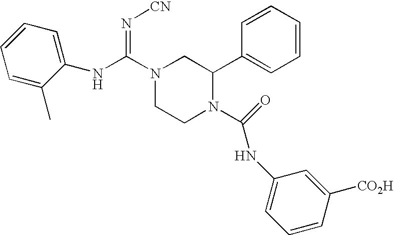 | 1.42 (d) | 483 (M + H)⁺ |
| 1.282 | 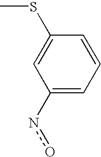 | C | 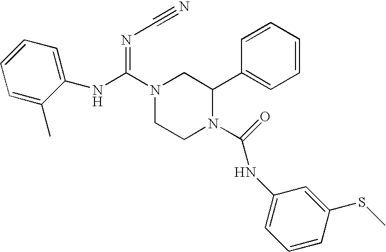 | 1.91 (d) | 485 (M + H)⁺ |
| 1.283 | 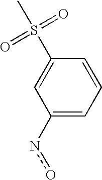 | C | 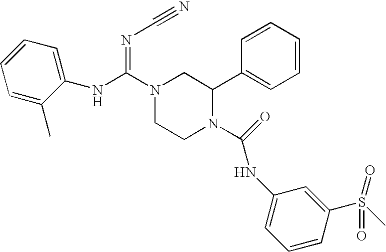 | 1.61 (d) | 517 (M + H)⁺ |
| 1.284 | 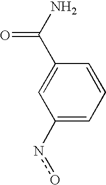 | C | 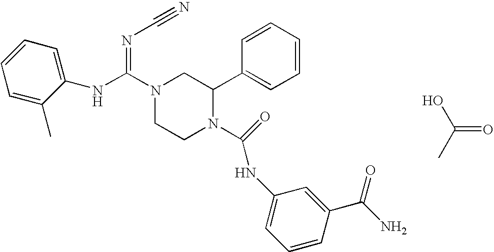 | 1.32 (d) | 482 (M + H)⁺ |
| 1.285 | 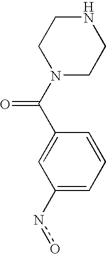 | C | 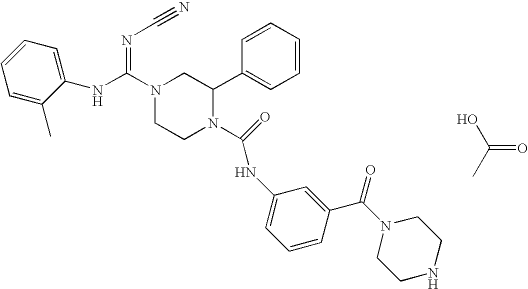 | 1.20 (d) | 551 (M + H)⁺ |
| 1.286 | 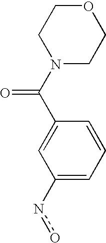 | C | 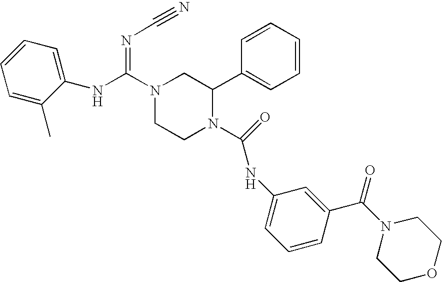 | 1.46 (d) | 552 (M + H)⁺ |
| 1.287 | 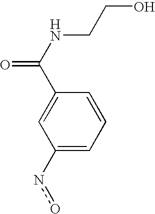 | C | 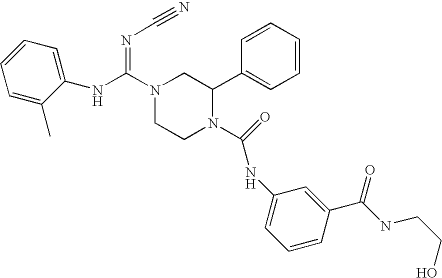 | 1.30 (d) | 526 (M + H)⁺ |
| 1.288 | 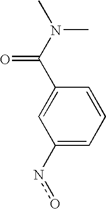 | C | 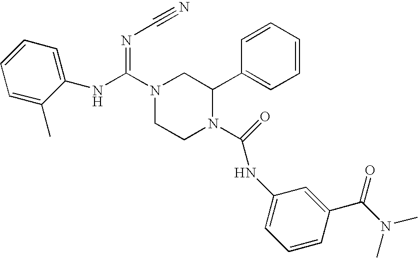 | 1.49 (d) | 510 (M + H)⁺ |
| 1.289 | 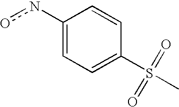 | C | 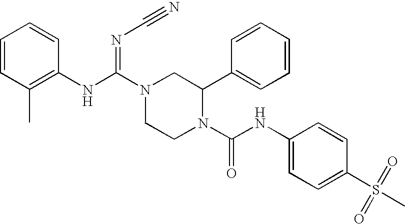 | 2.45 (a) | 517 (M + H)⁺ |
| 1.290 | 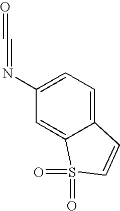 | C | 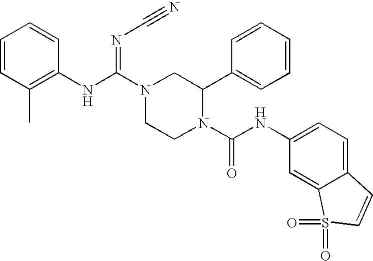 | 2.57 (a) | 526 (M + H)⁺ |
| 1.291 | 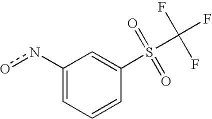 | C | 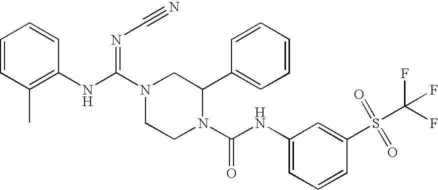 | 3.06 (a) | 571 (M + H)⁺ |
| 1.292 | 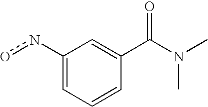 | C | 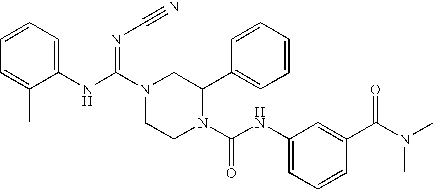 | 1.75 (b) | 508 (M − H)⁻ |
| 1.293 | 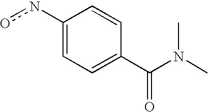 | C | 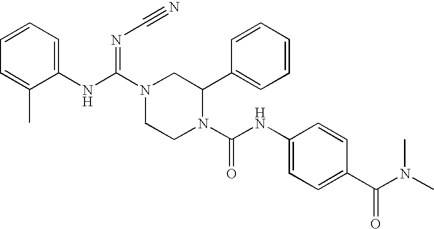 | 1.73 (b) | 508 (M − H)⁻ |

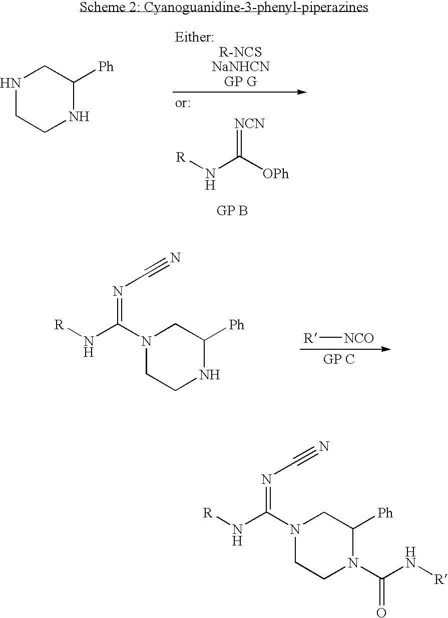

The compounds in table 2 were made according to either general procedure G or B, followed by general procedure C as illustrated in scheme 2. The specific procedure used for each compound is listed in table 2.

TABLE 2
Cyanoguanidine-3-phenyl-piperazines

| Ex # | R—NCS or 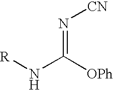 | GP | R′—NCO | Product | Rt/min (method) | m/z |
|---|---|---|---|---|---|---|
| 2.1 | 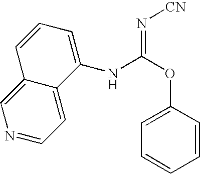 | B | 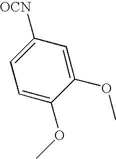 | 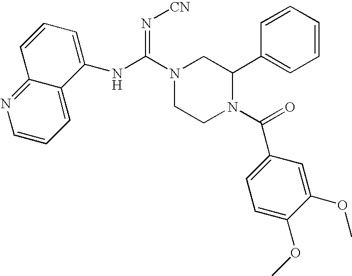 | 1.80 (i) | 521 (M + H)⁺ |
| 2.2 | 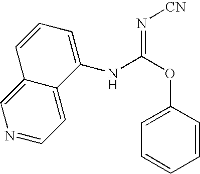 | B | 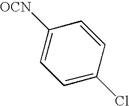 | 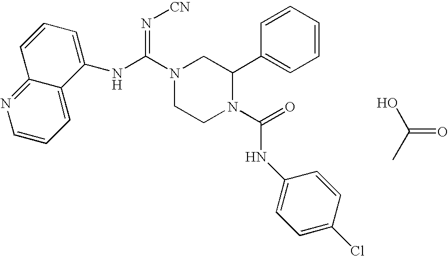 | 2.36 (i) | 510 (M + H)⁺ |
| 2.3 | 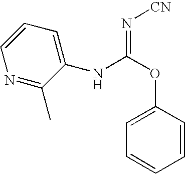 | B | 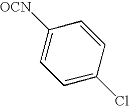 | 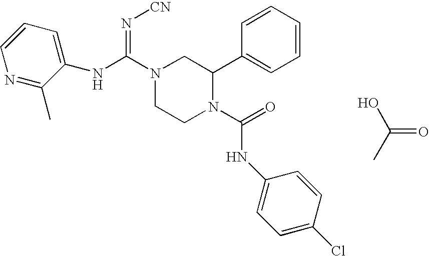 | 2.23 (i) | 474 (M + H)⁺ |
| 2.4 | 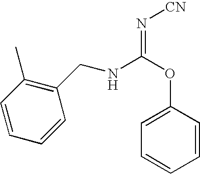 | B | 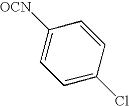 | 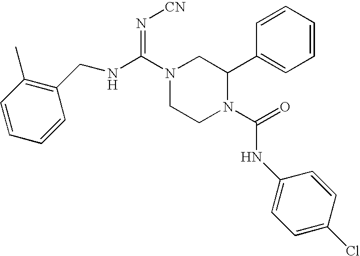 | 2.86 (i) | 487 (M + H)⁺ |
| 2.5 | 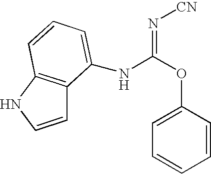 | B | 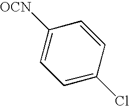 | 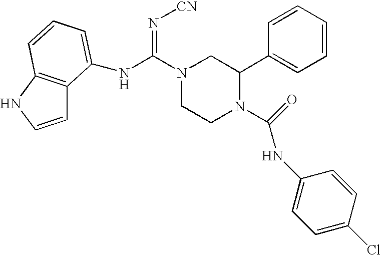 | 2.60 (i) | 498 (M + H)⁺ |
| 2.6 | 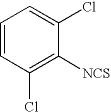 | G | 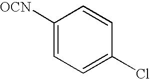 | 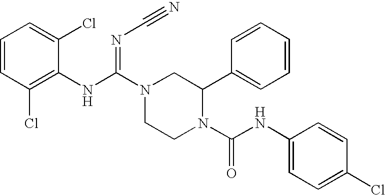 | 1.82 (i) | 525 (M − H)⁻ |
| 2.7 | 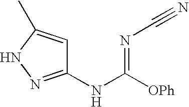 | B | 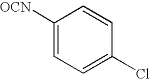 | 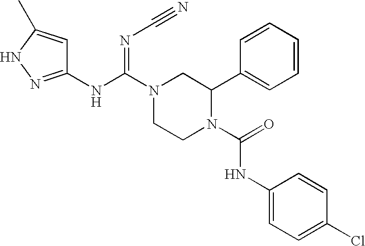 | 2.88 (i) | 463 (M + H)⁺ |
| 2.8 | 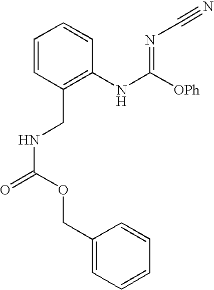 | B | 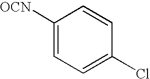 | 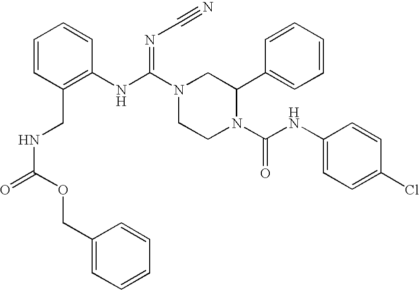 | 3.30 (b) | 622 (M + H)⁺ |
| 2.9 | 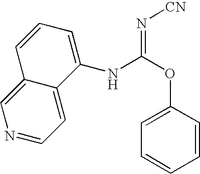 | B | 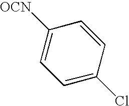 | 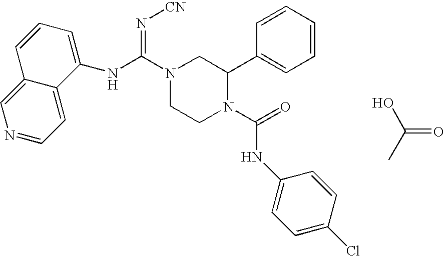 | 2.35 (i) | 510 (M + H)⁺ |
| 2.10 | 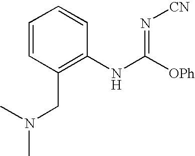 | B | 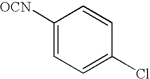 | 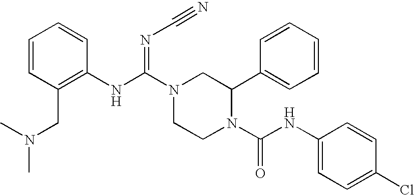 | 1.15 (b) | 335 (M + H)⁺ |
| 2.11 | 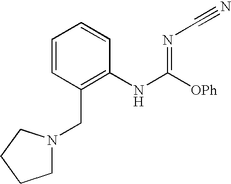 | B | 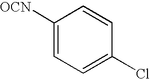 | 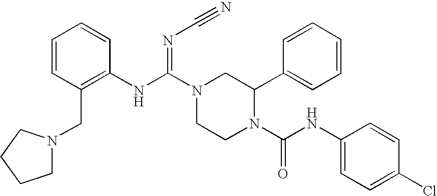 | 3.01 (k) | 540 (M + H)⁻ |
| 2.12 | 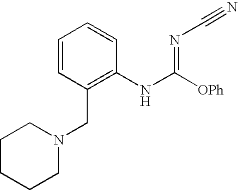 | B | 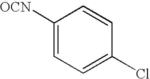 | 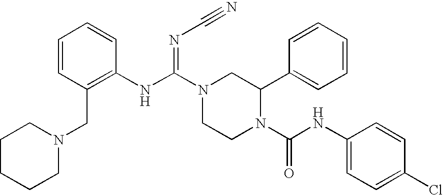 | 3.17 (k) | 556 (M + H)⁺ |
| 2.13 | 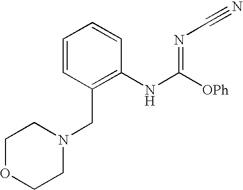 | B | 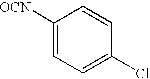 | 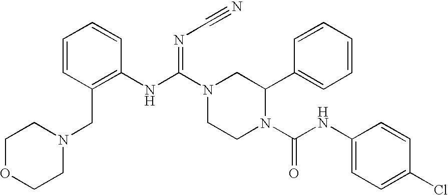 | 2.69 (k) | 558 (M + H)⁺ |
| 2.14 | 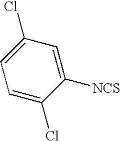 | G | 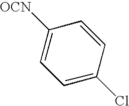 | 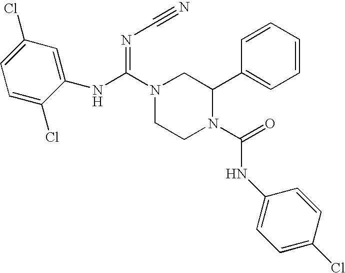 | 2.91 (i) | 527 (M + H)⁺ |
| 2.15 | 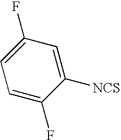 | G | 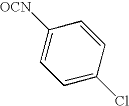 | 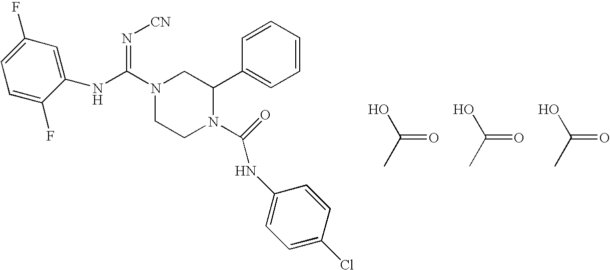 | 2.64 (i) | 495 (M + H)⁺ |
| 2.16 | 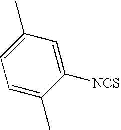 | G | 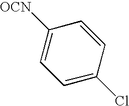 | 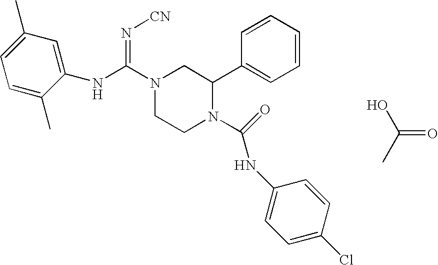 | 2.80 (i) | 487 (M + H)⁺ |
| 2.17 | 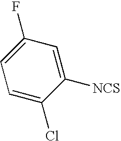 | G | 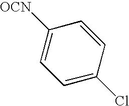 | 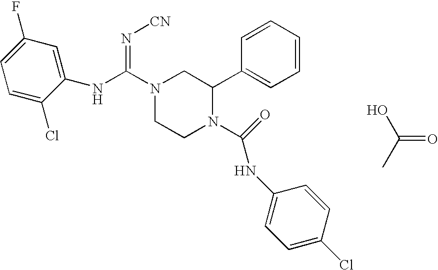 | 2.72 (i) | 511 (M + H)⁺ |
| 2.18 | 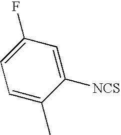 | G | 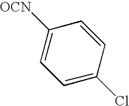 | 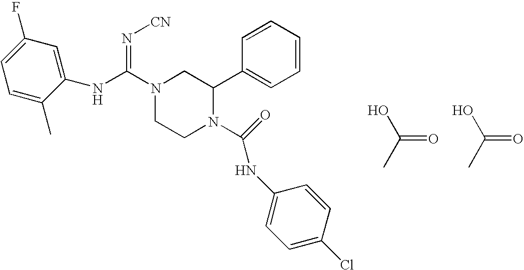 | 2.69 (i) | 491 (M + H)⁺ |
| 2.19 | 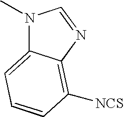 | G | 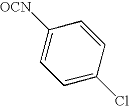 | 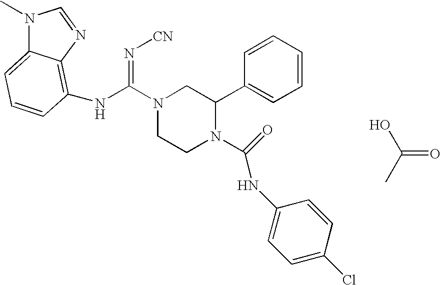 | 2.40 (i) | 513 (M + H)⁺ |
| 2.20 | 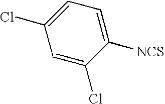 | G | 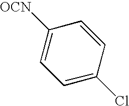 | 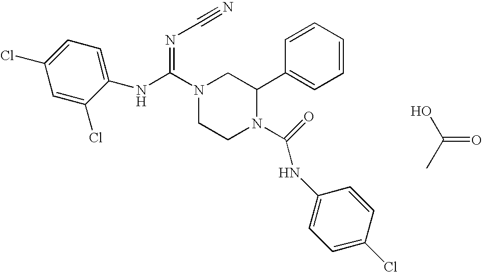 | 2.95 (i) | 527 (M + H)⁺ |
| 2.21 | 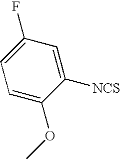 | G | 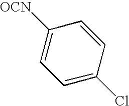 | 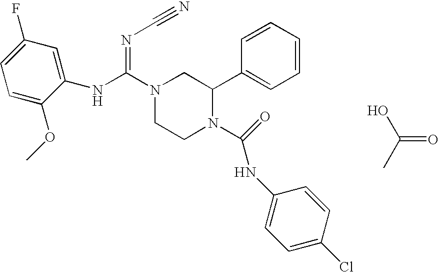 | 2.79 (i) | 507 (M + H)⁺ |
| 2.22 | 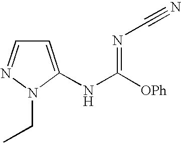 | B | 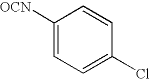 | 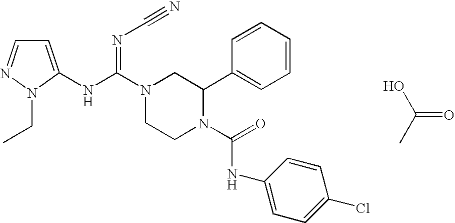 | 3.00 (i) | 477 (M + H)⁺ |
| 2.23 | 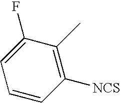 | G | 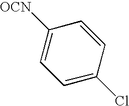 | 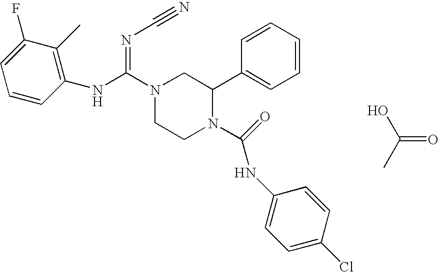 | 2.77 (i) | 491 (M + H)⁺ |
| 2.24 | 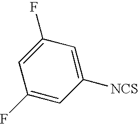 | G | 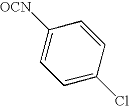 | 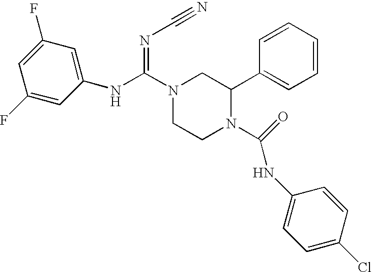 | 2.81 (i) | 495 (M + H)⁺ |
| 2.25 | 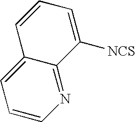 | G | 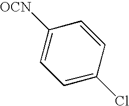 | 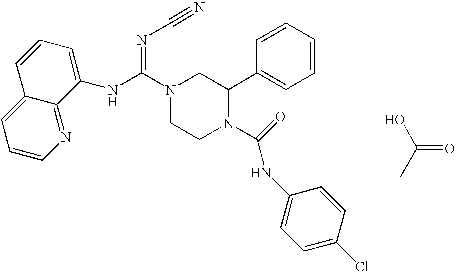 | 2.04 (i) | 510 (M + H)⁺ |
| 2.26 | 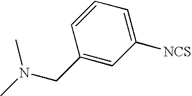 | G | 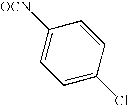 | 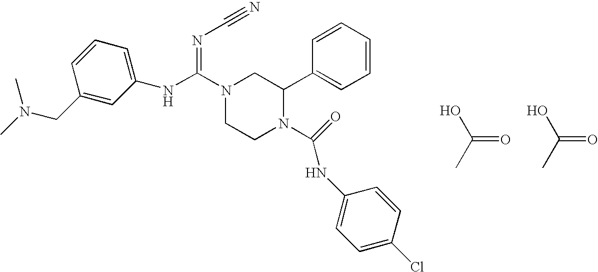 | 1.71 (i) | 516 (M + H)⁺ |
| 2.27 | 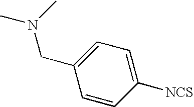 | G | 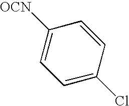 | 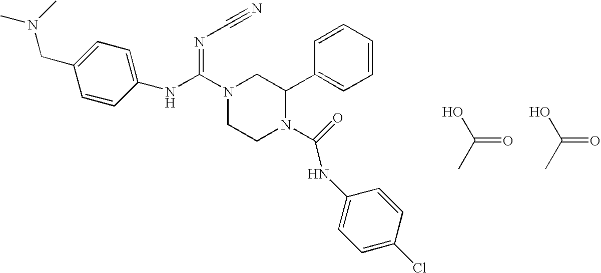 | 1.74 (i) | 516 (M + H)⁺ |
| 2.28 | 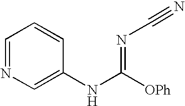 | B | 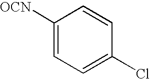 | 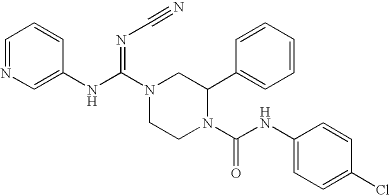 | 1.83 (d) | 460 (M + H)⁺ |
| 2.29 | 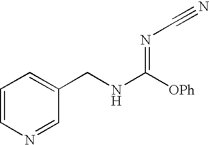 | B | 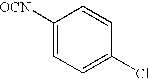 | 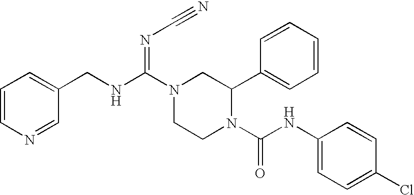 | 1.83 (d) | 474 (M + H)⁺ |
| 2.30 | 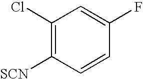 | G | 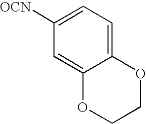 | 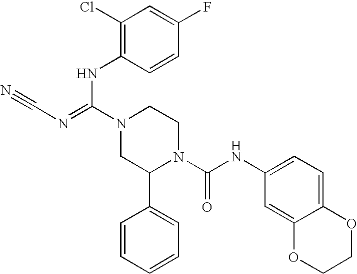 | 2.26 (k) | 536 (M + H)⁺ |
| 2.31 | 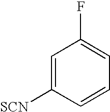 | G | 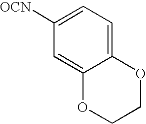 | 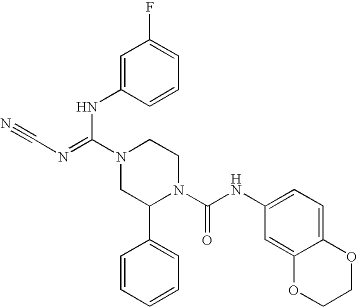 | 2.21 (k) | 502 (M + H)⁺ |
| 2.32 | 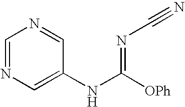 | B | 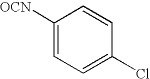 | 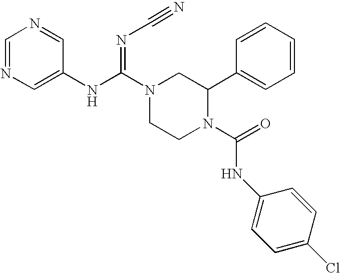 | 1.77 (d) | 461 (M + H)⁺ |
| 2.33 | 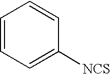 | G | 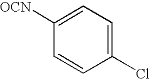 | 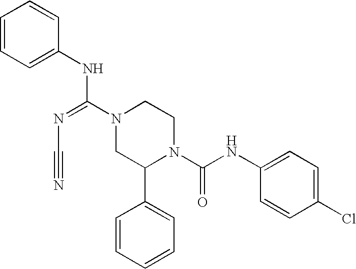 | 2.49 (k) | 460 (M + H)⁺ |
| 2.34 | 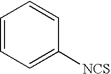 | G | 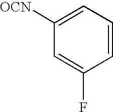 | 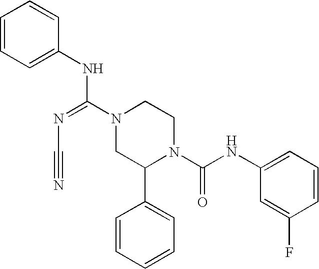 | 2.37 (k) | 444 (M + H)⁺ |
| 2.35 | 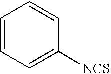 | G | 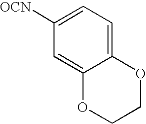 | 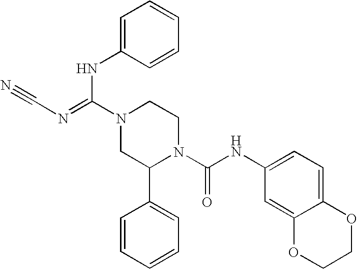 | 2.22 (k) | 484 (M + H)⁺ |
| 2.36 | 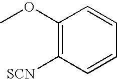 | G | 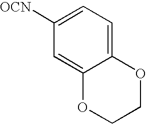 | 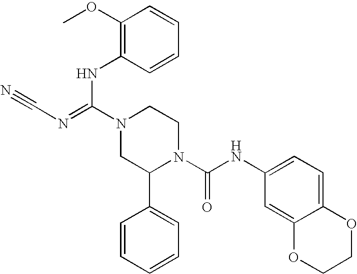 | 2.21 (k) | 514 (M + H)⁺ |
| 2.37 | 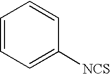 | G | 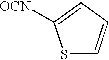 | 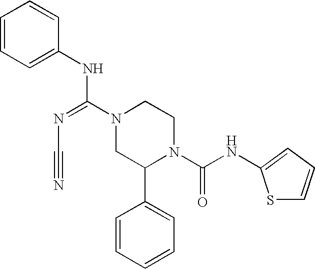 | 2.2 (k) | 432 (M + H)⁺ |
| 2.38 | 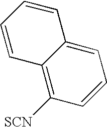 | G | 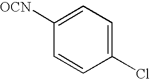 | 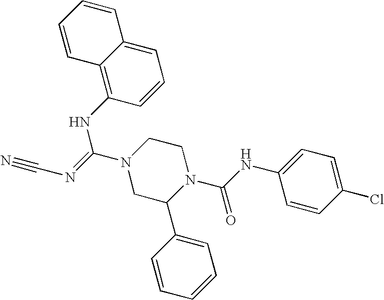 | 2.55 (k) | 510 (M + H)⁺ |
| 2.39 | 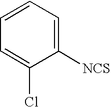 | G | 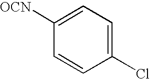 | 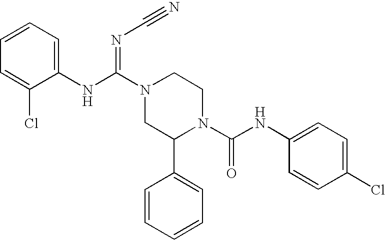 | 2.46 (k) | 494 (M + H)⁺ |
| 2.40 | 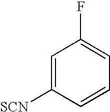 | G | 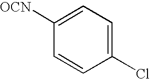 | 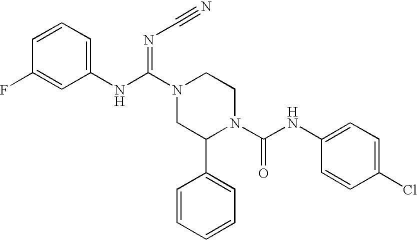 | 2.48 (k) | 478 (M + H)⁺ |
| 2.41 | 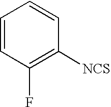 | G | 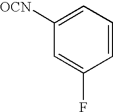 | 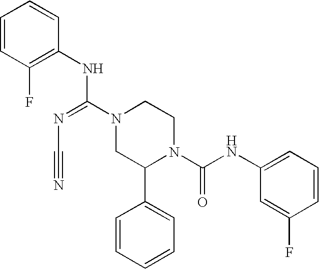 | 2.3 (k) | 462 (M + H)⁺ |
| 2.42 | 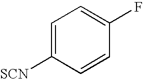 | G | 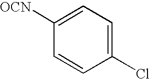 | 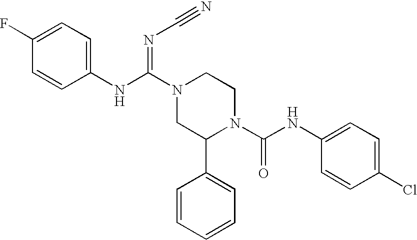 | 2.46 (k) | 478 (M + H)⁺ |
| 2.43 | 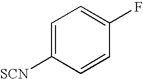 | G | 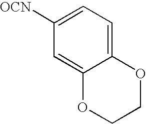 | 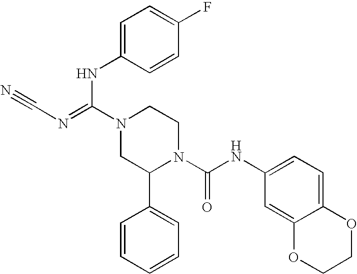 | 2.22 (k) | 502 (M + H)⁺ |
| 2.44 | 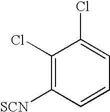 | G | 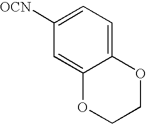 | 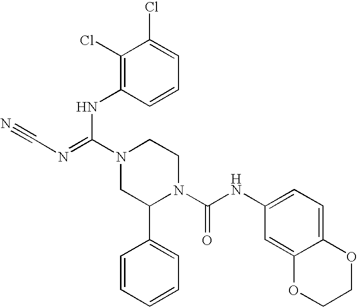 | 2.35 (k) | 552 (M + H)⁺ |
| 2.45 | 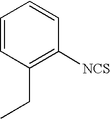 | G | 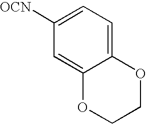 | 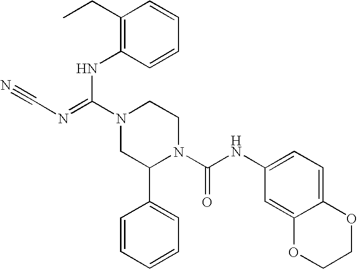 | 2.3 (k) | 512 (M + H)⁺ |
| 2.46 | 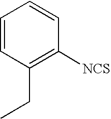 | G | 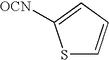 | 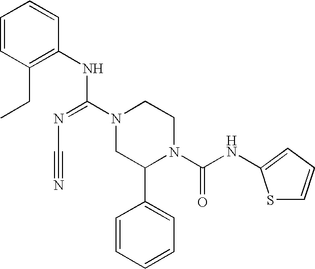 | 2.32 (k) | 460 (M + H)⁺ |
| 2.47 | 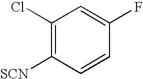 | G | 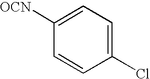 | 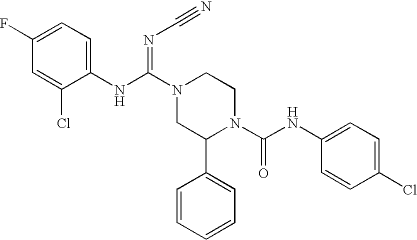 | 2.52 (k) | 512 (M + H)⁺ |
| 2.48 | 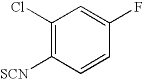 | G | 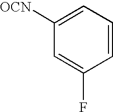 | 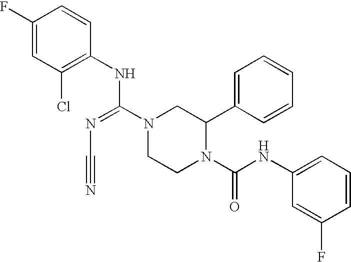 | 2.42 (k) | 496 (M + H)⁺ |
| 2.49 | 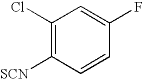 | G | 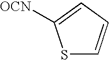 | 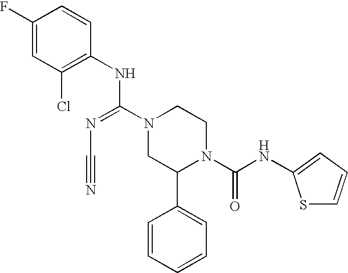 | 2.29 (k) | 484 (M + H)⁺ |
| 2.50 | 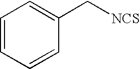 | G | 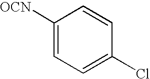 | 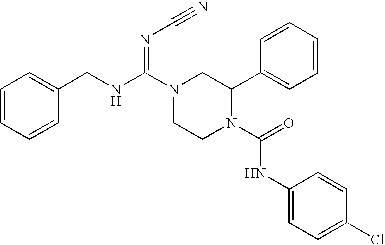 | 1.99 (d) | 471 (M − H)⁻ |
| 2.51 | 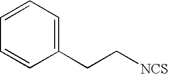 | G | 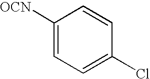 | 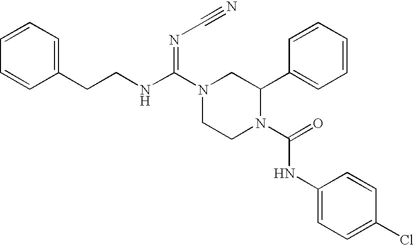 | 2.05 (d) | 485 (M − H)⁻ |
| 2.52 | 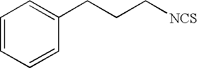 | G | 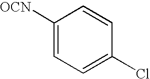 | 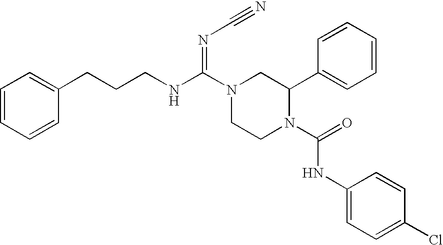 | 2.11 (d) | 499 (M − H)⁻ |
| 2.53 | 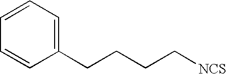 | G | 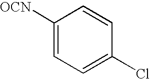 | 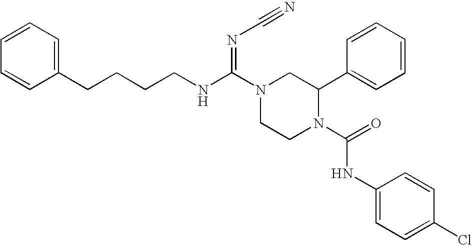 | 2.20 (d) | 513 (M − H)⁻ |
| 2.54 | 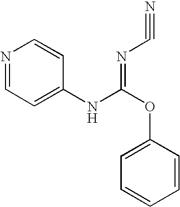 | B | 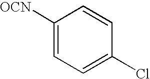 | 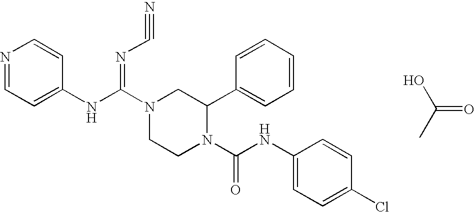 | 1.63 (d) | 460 (M + H)⁺ |
| 2.55 | 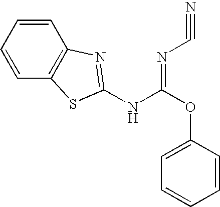 | B | 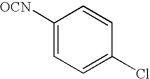 | 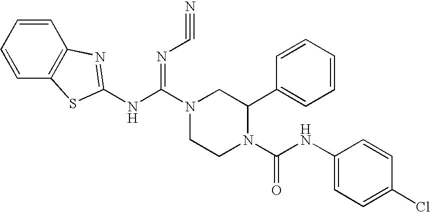 | 6.2 (e) | 516 (M + H)⁺ |
| 2.56 | 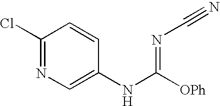 | B | 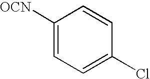 | 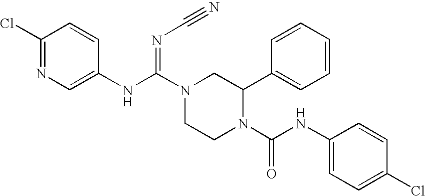 | 1.85 (d) | 494 (M + H)⁺ |
| 2.57 | 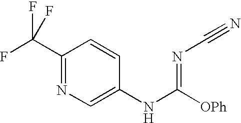 | B | 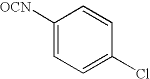 | 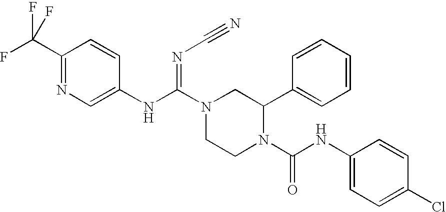 | 1.99 (d) | 526 (M − H)⁻ |
| 2.58 | 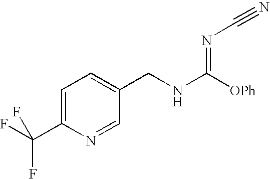 | B | 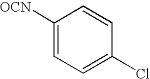 | 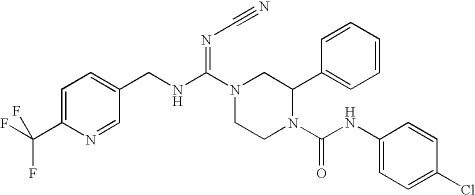 | 2.13 (d) | 542 (M + H)⁺ |
| 2.59 | 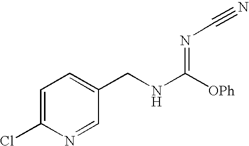 | B | 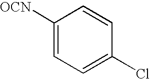 | 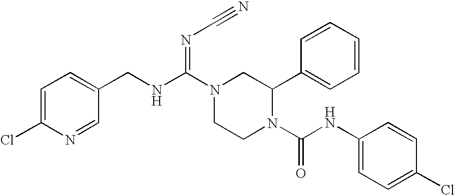 | 2.02 (d) | 508 (M + H)⁺ |
| 2.60 | 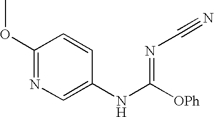 | B | 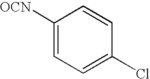 | 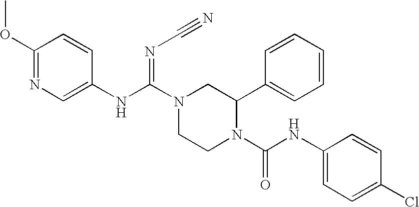 | 1.79 (d) | 490 (M + H)⁺ |
| 2.61 | 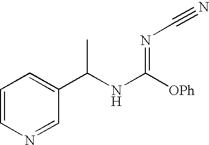 | B | 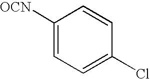 | 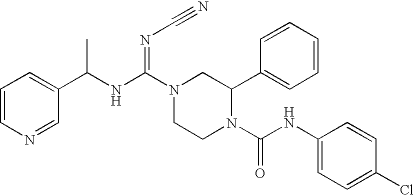 | 1.77 (d) | 486 (M − H)⁻ |
| 2.62 | 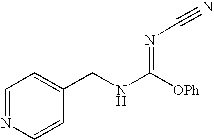 | B | 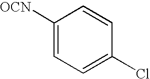 | 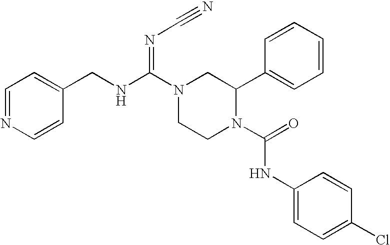 | 1.61 (d) | 474 (M + H)⁺ |
| 2.63 | 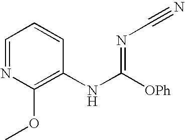 | B | 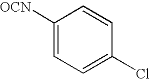 | 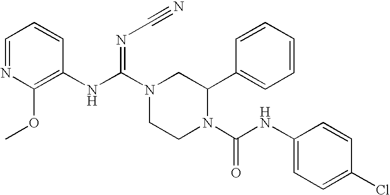 | 1.87 (d) | 490 (M + H)⁺ |
| 2.64 | 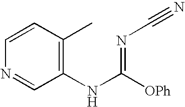 | B | 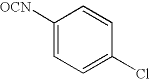 | 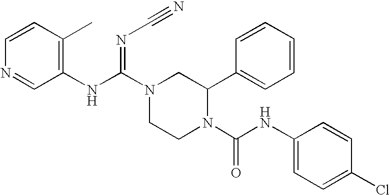 | 1.73 (d) | 474 (M + H)⁺ |
| 2.65 | 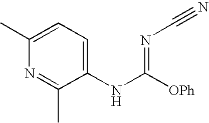 | B | 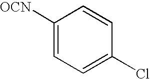 | 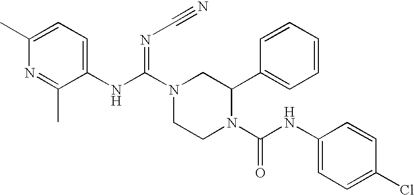 | 1.83 (d) | 488 (M + H)⁺ |
| 2.66 | 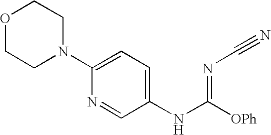 | B | 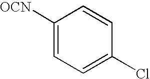 | 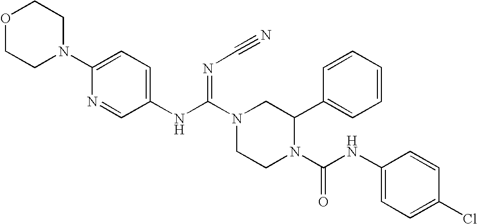 | 1.88 (d) | 545 (M + H)⁺ |
| 2.67 | 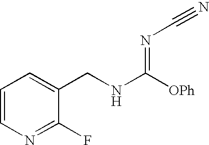 | B | 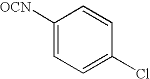 | 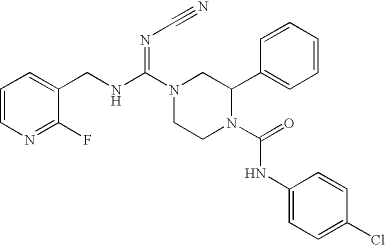 | 1.83 (d) | 492 (M + H)⁺ |
| 2.68 | 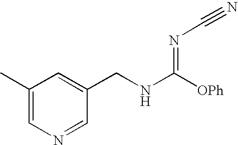 | B | 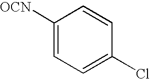 | 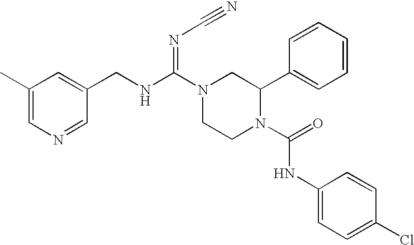 | 3.05 (d) | 488 (M + H)⁺ |
| 2.69 | 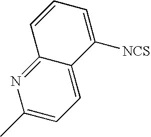 | G | 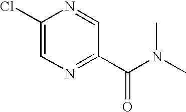 | 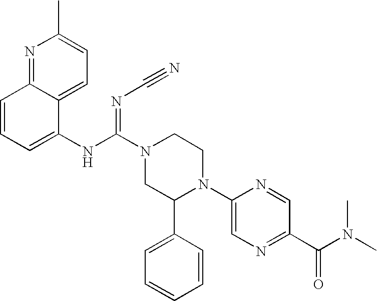 | 1.82 (a) | 520 (M + H)⁺ |
| 2.70 | 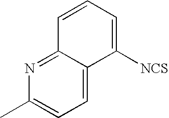 | G | 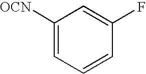 | 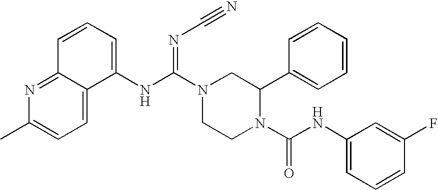 | 2.28 (a) | 506 (M − H)⁻ |
| 2.71 | 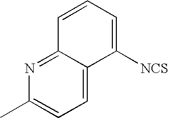 | G | 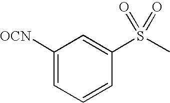 | 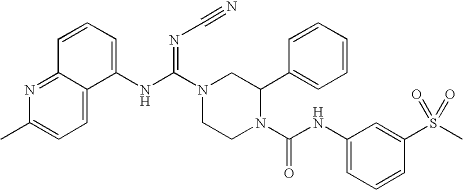 | 1.97 (a) | 566 (M − H)⁻ |

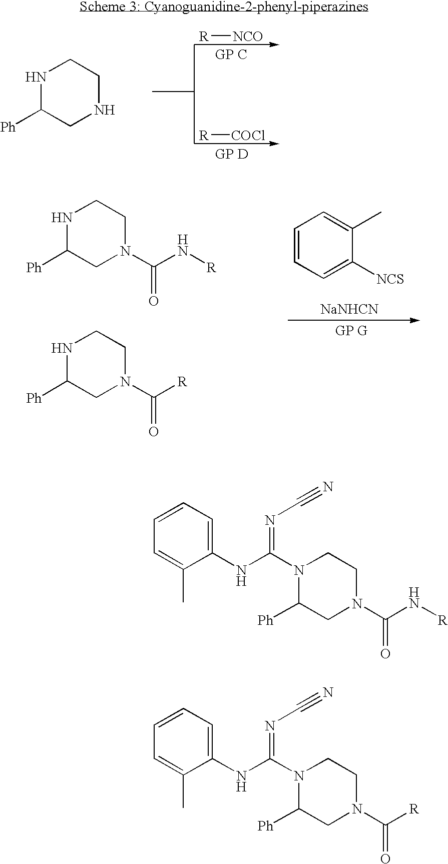

The compounds in table 3 were made according to either general procedure C or D, followed by general procedure G as illustrated in scheme 3. The specific procedure used for each compound is listed in table 3.

TABLE 3
Cyanoguanidine-2-phenyl-piperazines

| | | | | Rt/min | |
|---|---|---|---|---|---|
| Ex # | Reagent | GP | Product | (method) | m/z |
| 3.1 | 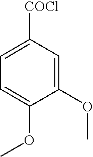 | D | 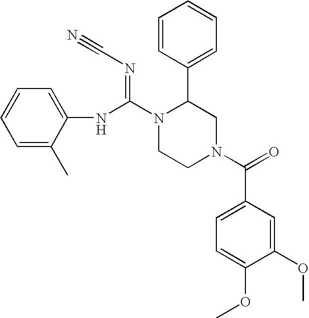 | 2.17 (i) | 484 (M + H)⁺ |
| 3.2 | 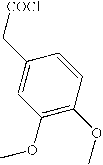 | D | 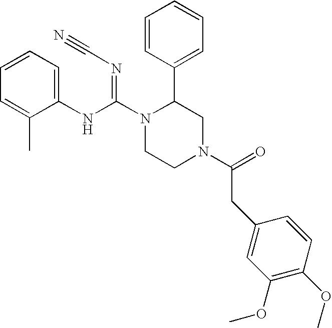 | 2.22 (i) | 498 (M + H)⁺ |
| 3.3 | 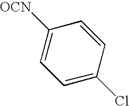 | C | 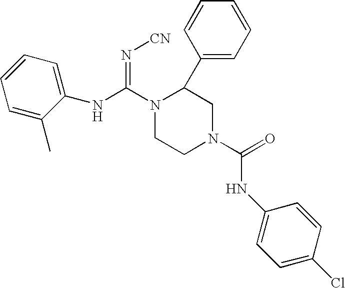 | 2.74 (i) | 473 (M + H)⁺ |

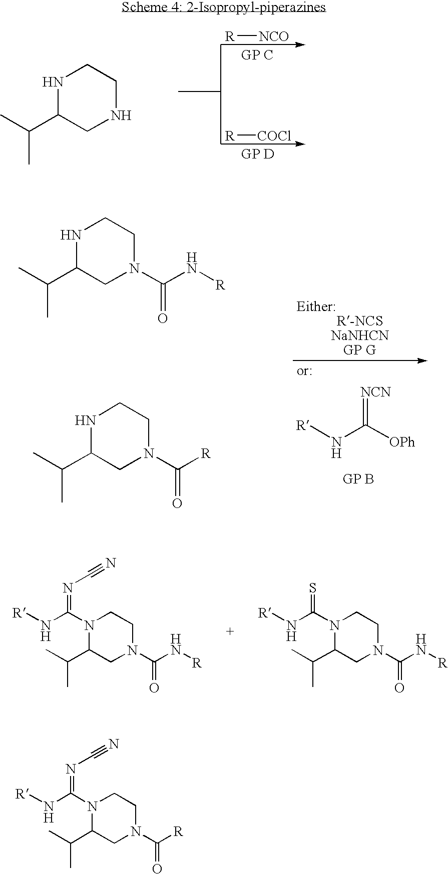

The compounds in table 4 were made according to either general procedure C or D, followed by general procedure G or B as illustrated in scheme 4. The specific procedures used for each compound are listed in table 4.

TABLE 4
2-Isopropyl-piperazines

| Ex # | R—NCO or R-COCl | GP | R′—NCS or 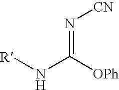 | GP | Product | Rt/min (method) | m/z |
|---|---|---|---|---|---|---|---|
| 4.1 | 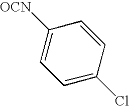 | C | 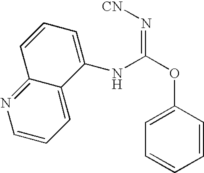 | B | 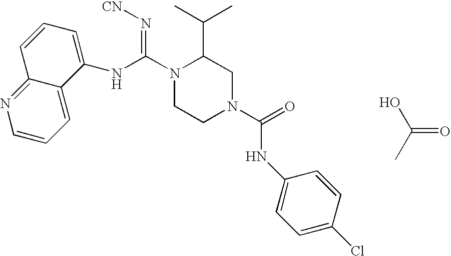 | 2.34 (i) | 476 (M + H)⁺ |
| 4.2 | 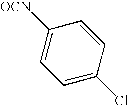 | C | 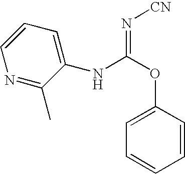 | B | 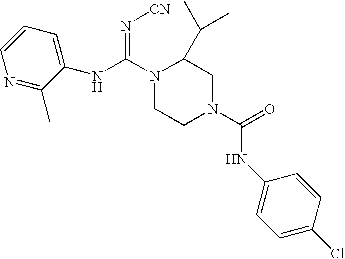 | 2.25 (i) | 440 (M + H)⁺ |
| 4.3 | 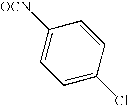 | C | 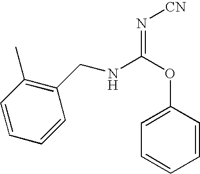 | B | 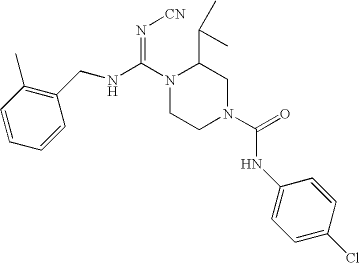 | 2.86 (i) | 453 (M + H)⁺ |
| 4.4 | 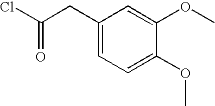 | D | 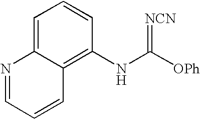 | B | 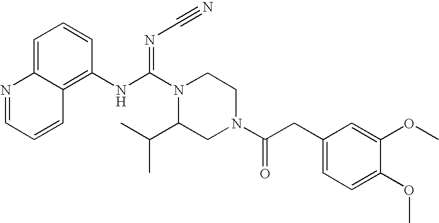 | 1.80 (i) | 499 (M − H)⁻ |
| 4.5 | 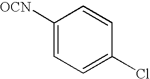 | C | 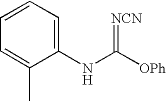 | B | 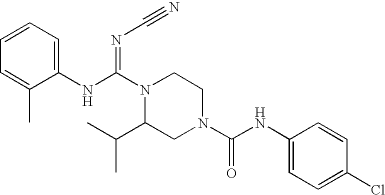 | 2.70 (i) | 439 (M + H)⁺ |
| 4.6 | 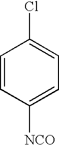 | C | 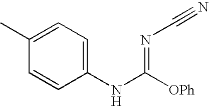 | B | 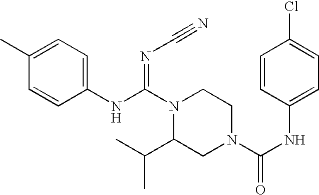 | 2.51 (k) | 439 (M + H)⁺ |
| 4.7 | 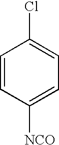 | C | 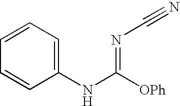 | B | 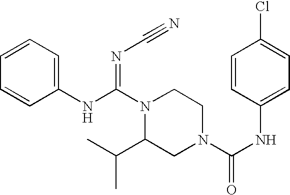 | 2.38 (k) | 425 (M + H)⁺ |
| 4.8 | 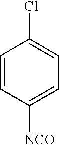 | C | 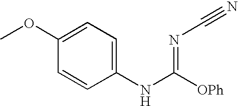 | B | 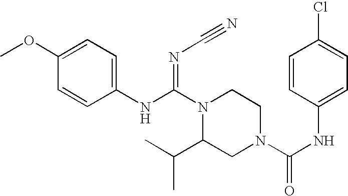 | 2.4 (k) | 455 (M + H)⁺ |
| 4.9 | 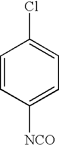 | C | 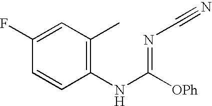 | B | 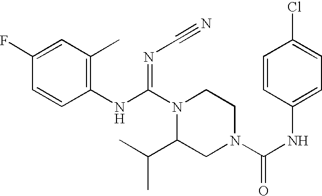 | 2.51 (k) | 457 (M + H)⁺ |
| 4.10 | 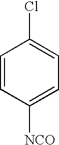 | C | 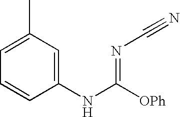 | B | 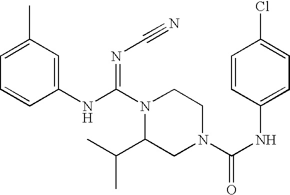 | 2.5 (k) | 439 (M + H)⁺ |
| 4.11 | 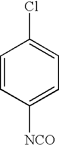 | C | 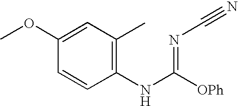 | B | 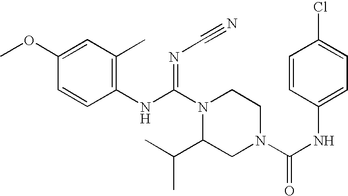 | 2.44 (k) | 469 (M + H)⁺ |
| 4.12 | 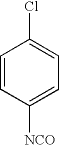 | C | 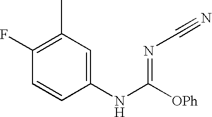 | B | 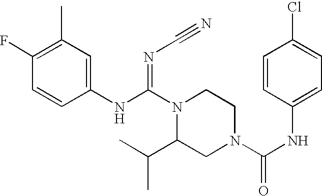 | 2.48 (k) | 457 (M + H)⁺ |
| 4.13 | 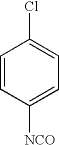 | C | 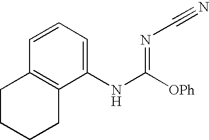 | B | 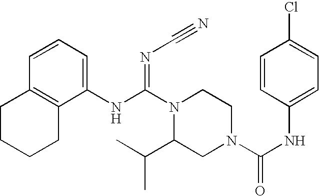 | 2.69 (k) | 479 (M + H)⁺ |
| 4.14 | 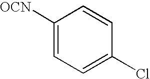 | C | 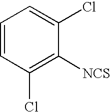 | G | 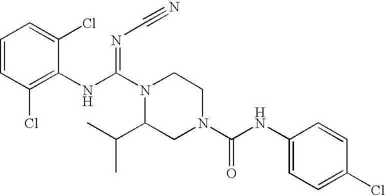 | 2.77 (i) | 493 (M − H)⁺ |
| 4.15 | 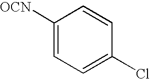 | C | 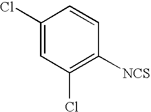 | G | 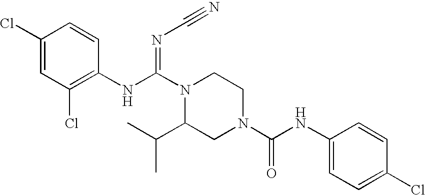 | 2.62 | 491 (M − H)⁻ |
| 4.16 | 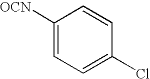 | C | 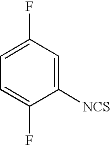 | G | 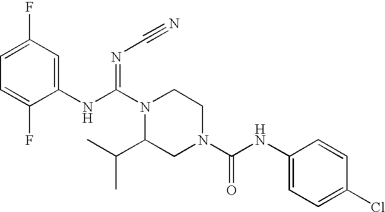 | 1.93 (d) | 459 (M − H)⁻ |
| 4.17 | 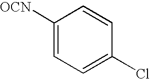 | C | 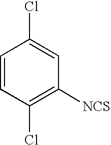 | G | 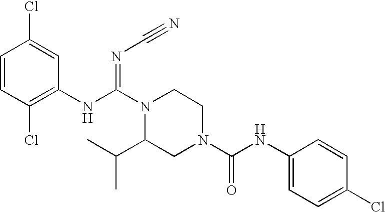 | 2.89 (i) | 491 (M − H)⁻ |
| 4.18 | 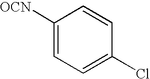 | C | 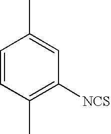 | G | 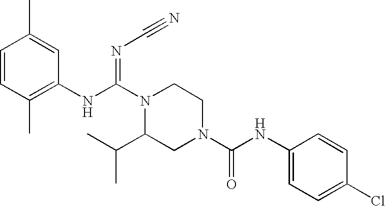 | 2.88 (i) | 451 (M − H)⁻ |
| 4.19 | 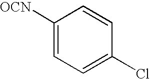 | C | 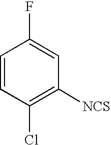 | G | 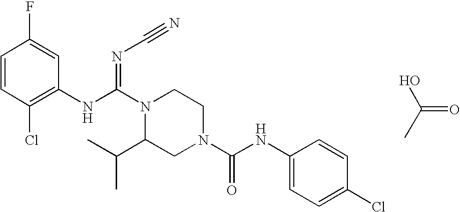 | 2.76 (i) | 475 (M − H)⁻ |
| 4.20 | 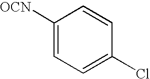 | C | 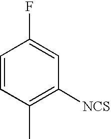 | G | 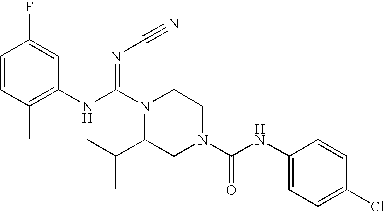 | 2.73 (i) | 455 (M − H)⁻ |
| 4.21 | 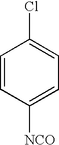 | C | 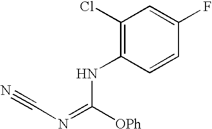 | B | 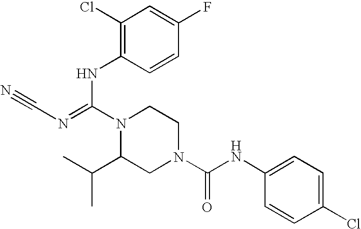 | 2.48 (k) | 477 (M + H)⁺ |
| 4.22 | 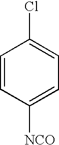 | C | 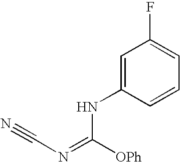 | B | 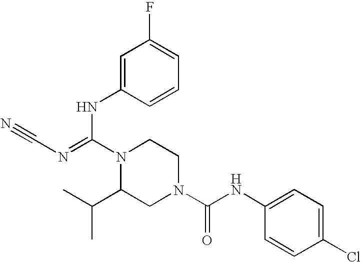 | 2.43 (k) | 443 (M + H)⁺ |
| 4.23 | 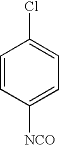 | C | 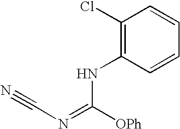 | B | 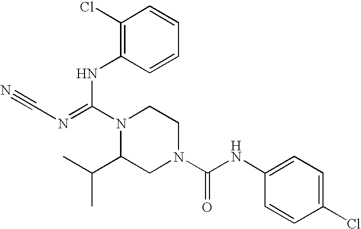 | 2.42 (k) | 459 (M + H)⁺ |
| 4.24 | 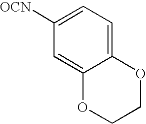 | C | 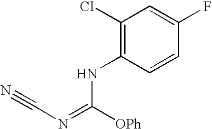 | B | 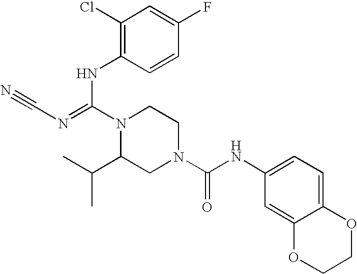 | 2.17 (k) | 501 (M + H)⁺ |
| 4.25 | 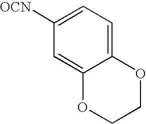 | C | 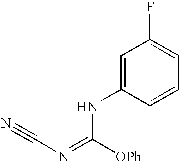 | B | 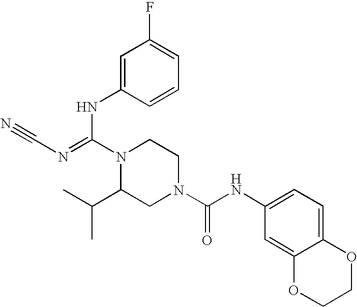 | 2.16 (k) | 467 (M + H)⁺ |
| 4.26 | 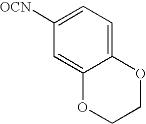 | C | 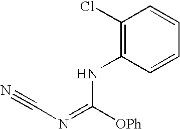 | B | 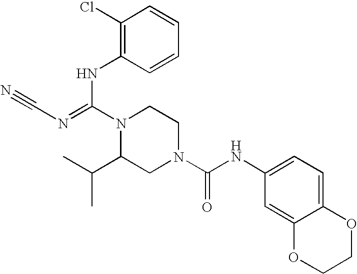 | 2.14 (k) | 483 (M + H)⁺ |
| 4.27 | 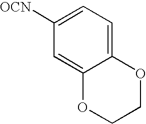 | C | 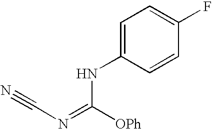 | B | 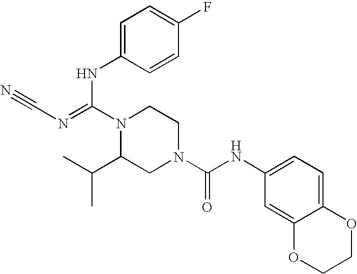 | 2.14 (k) | 467 (M + H)⁺ |
| 4.28 | 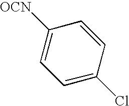 | C | 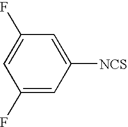 | G | 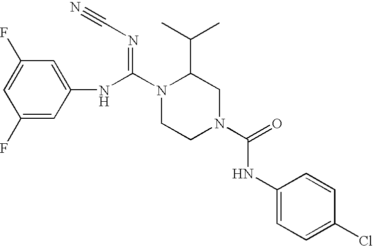 | 2.86 (d) | 453 (M + H)⁺ |
| 4.29 | 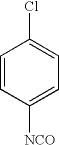 | C | 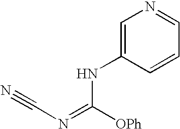 | B | 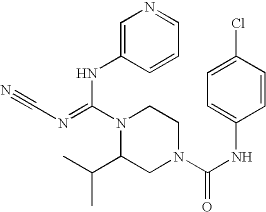 | 2.07 (k) | 426 (M + H)⁺ |
| 4.30 | 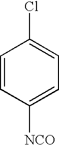 | C | 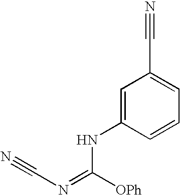 | B | 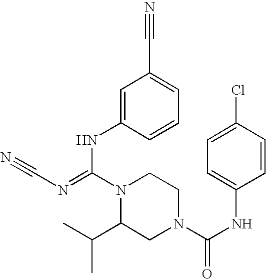 | 2.35 (k) | 450 (M + H)⁺ |
| 4.31 | 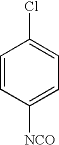 | C | 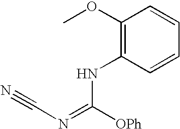 | B | 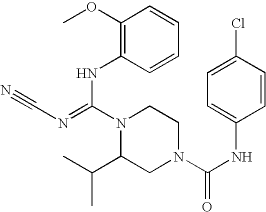 | 2.39 (k) | 455 (M + H)⁺ |
| 4.32 | 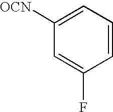 | C | 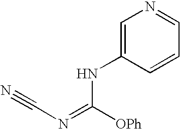 | B | 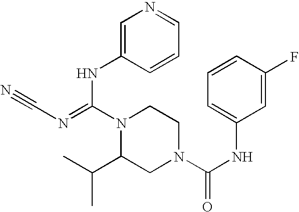 | 1.97 (k) | 410 (M + H)⁺ |
| 4.33 | 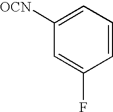 | C | 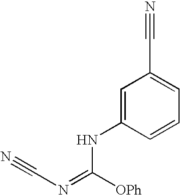 | B | 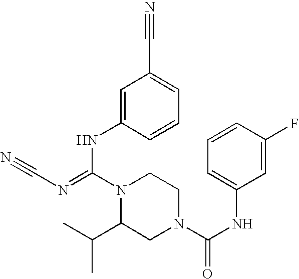 | 2.24 (k) | 434 (M + H)⁺ |
| 4.34 | 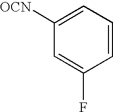 | C | 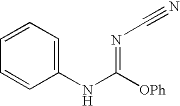 | B | 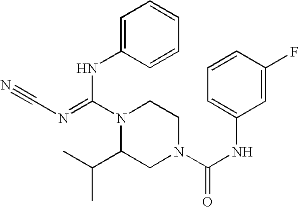 | 2.27 (k) | 409 (M + H)⁺ |
| 4.35 | 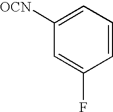 | C | 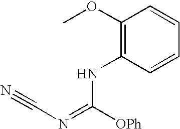 | B | 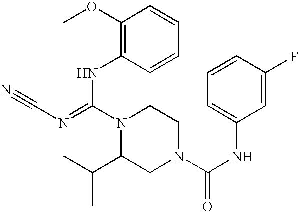 | 2.28 (k) | 439 (M + H)⁺ |
| 4.36 | 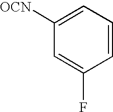 | C | 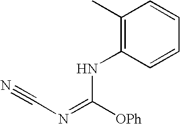 | B | 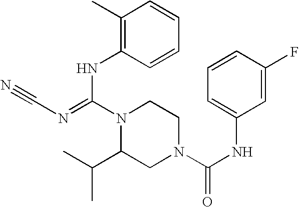 | 2.31 (k) | 423 (M + H)⁺ |
| 4.37 | 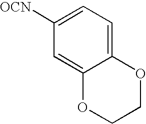 | C | 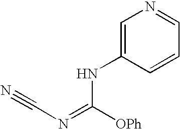 | B | 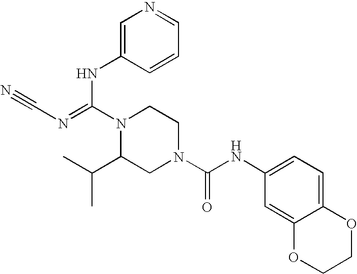 | 1.82 (k) | 450 (M + H)⁺ |
| 4.38 | 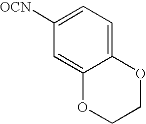 | C | 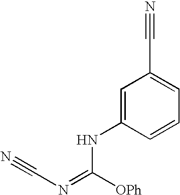 | B | 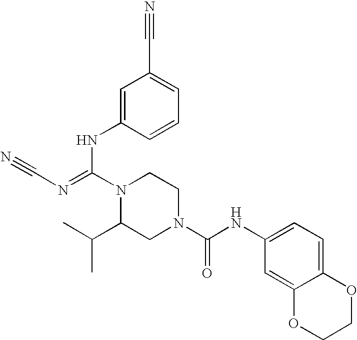 | 2.09 (k) | 474 (M + H)⁺ |
| 4.39 | 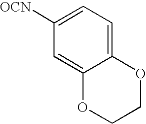 | C | 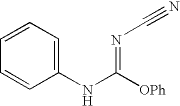 | B | 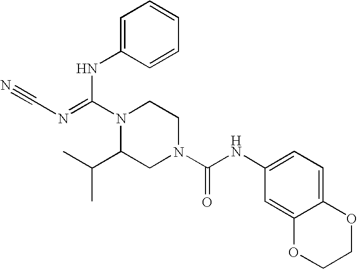 | 2.09 (k) | 449 (M + H)⁺ |
| 4.40 | 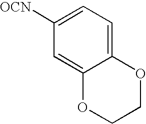 | C | 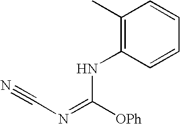 | B | 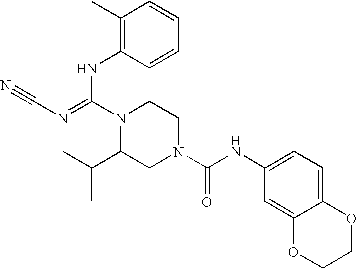 | 2.13 (k) | 463 (M + H)⁺ |
| 4.41 | 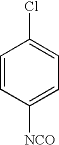 | C | 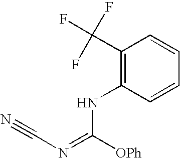 | B | 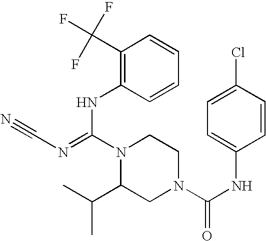 | 2.47 (k) | 493 (M + H)⁺ |
| 4.42 | 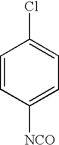 | C | 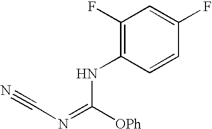 | B | 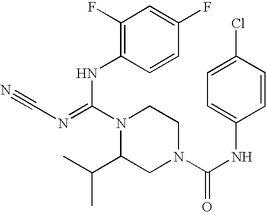 | 2.4 (k) | 461 (M + H)⁺ |
| 4.43 | 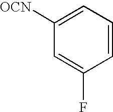 | C | 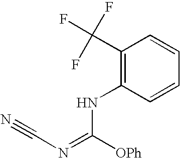 | B | 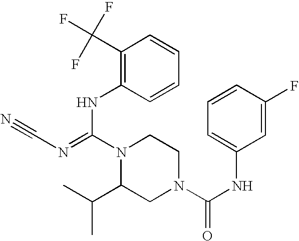 | 2.37 (k) | 477 (M + H)⁺ |
| 4.44 | 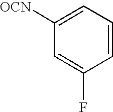 | C | 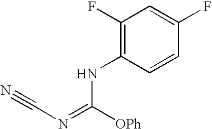 | B | 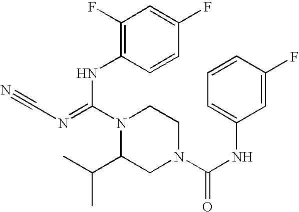 | 2.29 (k) | 445 (M + H)⁺ |
| 4.45 | 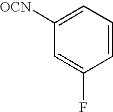 | C | 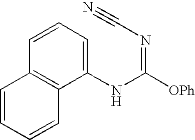 | B | 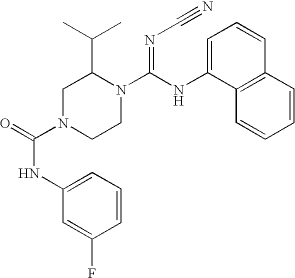 | 2.42 (k) | 459 (M + H)⁺ |
| 4.46 | 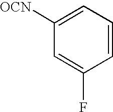 | C | 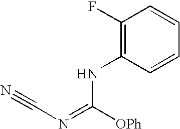 | B | 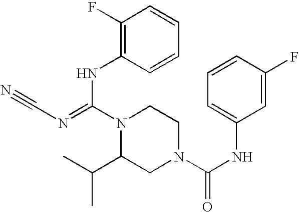 | 2.26 (k) | 427 (M + H)⁺ |
| 4.47 | 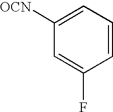 | C | 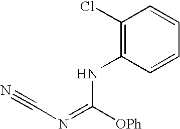 | B | 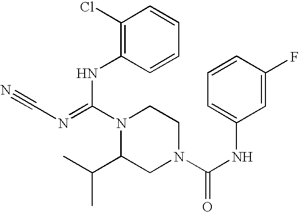 | 2.31 (k) | 443 (M + H)⁺ |
| 4.48 | 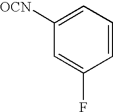 | C | 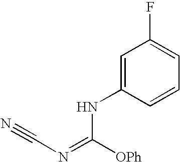 | B | 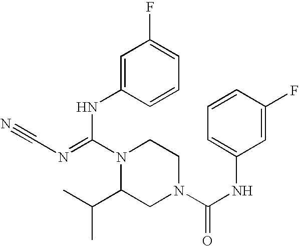 | 2.33 (k) | 427 (M + H)⁺ |
| 4.49 | 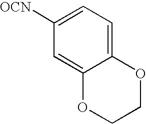 | C | 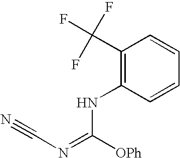 | B | 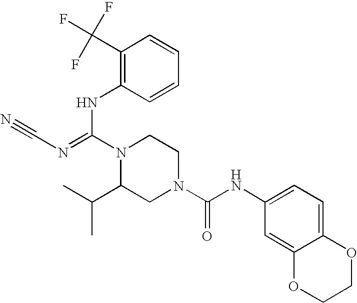 | 2.2 (k) | 517 (M + H)⁺ |
| 4.50 | 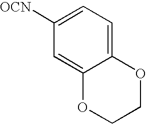 | C | 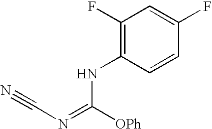 | B | 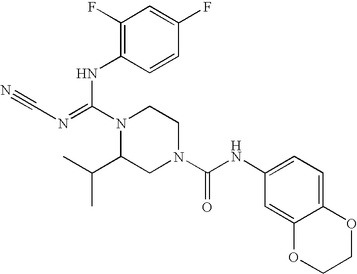 | 2.13 (k) | 485 (M + H)⁺ |
| 4.51 | 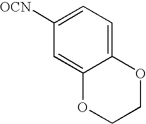 | C | 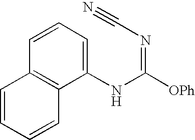 | B | 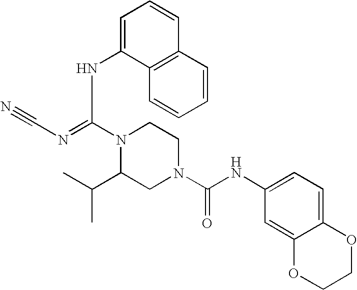 | 2.25 (k) | 499 (M + H)⁺ |
| 4.52 | 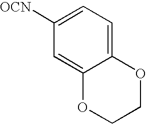 | C | 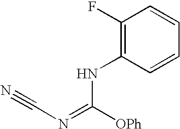 | B | 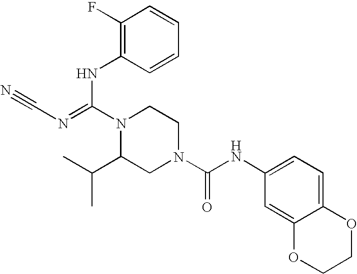 | 2.09 (k) | 467 (M + H)⁺ |
| 4.53 | 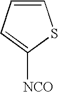 | C | 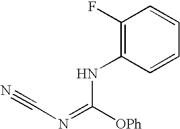 | B | 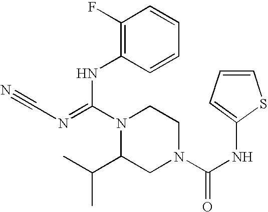 | 2.16 (k) | 415 (M + H)⁺ |
| 4.54 | 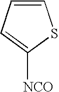 | C | 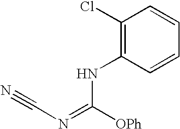 | B | 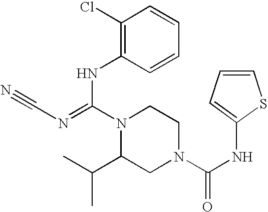 | 2.22 (k) | 431 (M + H)⁺ |
| 4.55 | 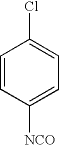 | C | 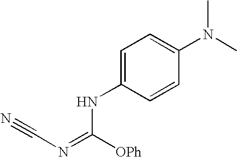 | B | 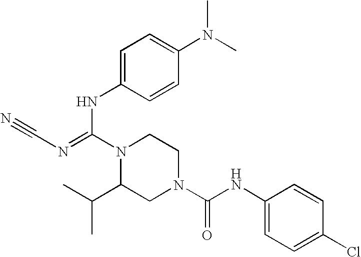 | 2.48 (k) | 468 (M + H)⁺ |
| 4.56 | 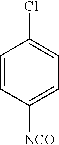 | C | 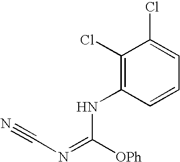 | B | 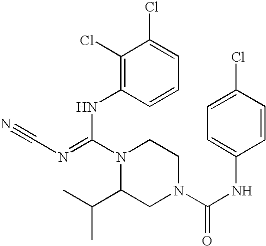 | 2.58 (k) | 493 (M + H)⁺ |
| 4.57 | 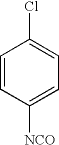 | C | 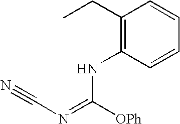 | B | 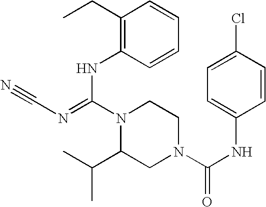 | 2.54 (k) | 453 (M + H)⁺ |
| 4.58 | 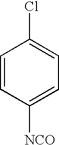 | C | 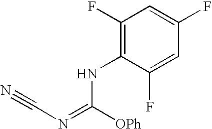 | B | 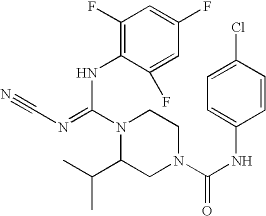 | 2.45 (k) | 479 (M + H)⁺ |
| 4.59 | 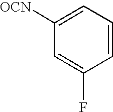 | C | 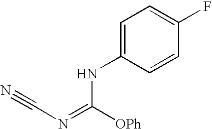 | B | 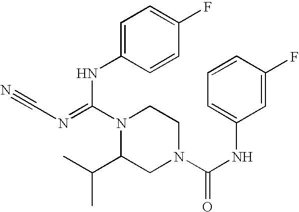 | 2.32 (k) | 427 (M + H)⁺ |
| 4.60 | 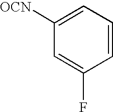 | C | 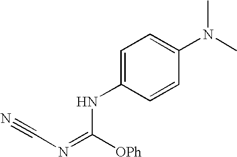 | B | 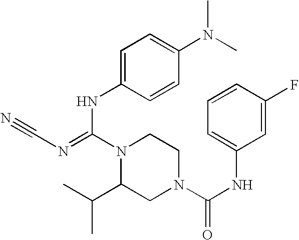 | 2.36 (k) | 452 (M + H)⁺ |
| 4.61 | 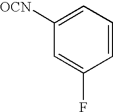 | C | 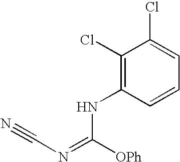 | B | 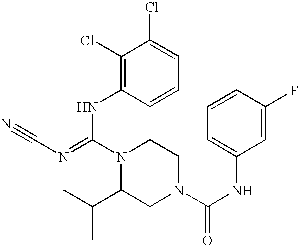 | 2.47 (k) | 477 (M + H)⁺ |
| 4.62 | 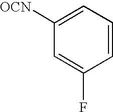 | C | 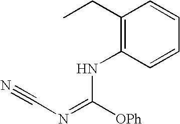 | B | 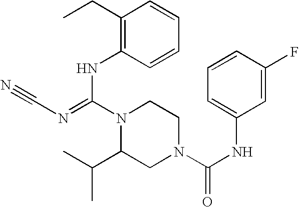 | 2.42 (k) | 437 (M + H)⁺ |
| 4.63 | 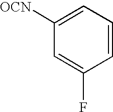 | C | 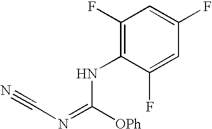 | B | 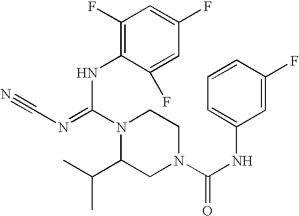 | 2.34 (k) | 463 (M + H)⁺ |
| 4.64 | 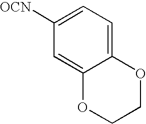 | C | 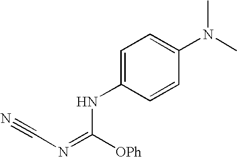 | B | 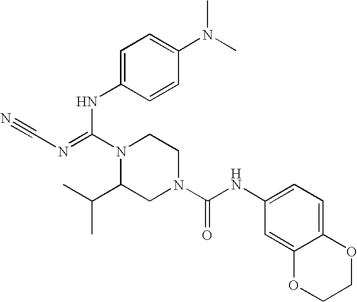 | 2.17 (k) | 492 (M + H)⁺ |
| 4.65 | 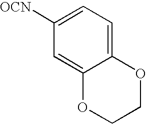 | C | 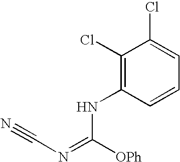 | B | 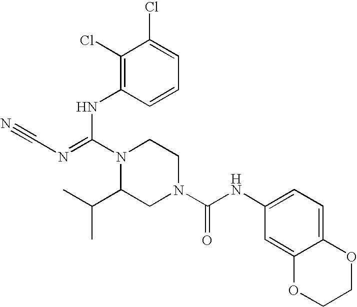 | 2.3 (k) | 517 (M + H)⁺ |
| 4.66 | 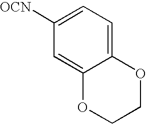 | C | 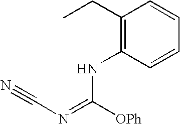 | B | 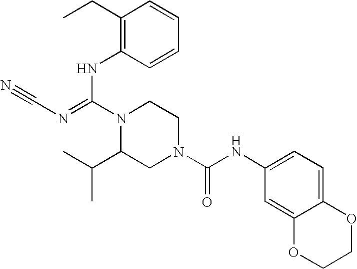 | 2.24 (k) | 477 (M + H)⁺ |
| 4.67 | 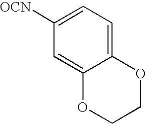 | C | 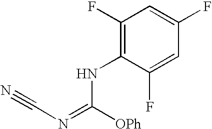 | B | 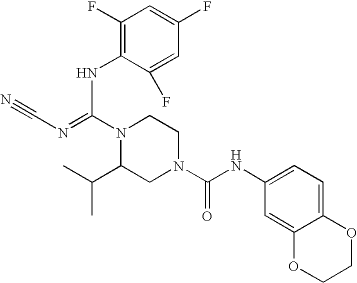 | 2.17 (k) | 503 (M + H)⁺ |
| 4.68 | 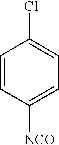 | C | 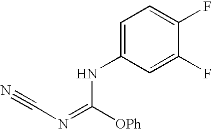 | B | 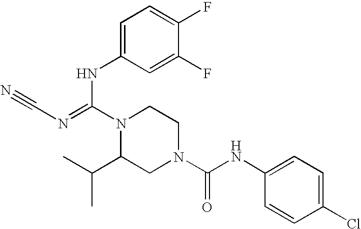 | 2.49 (k) | 461 (M + H)⁺ |
| 4.69 | 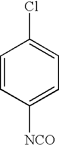 | C | 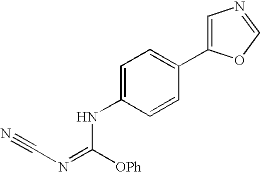 | B | 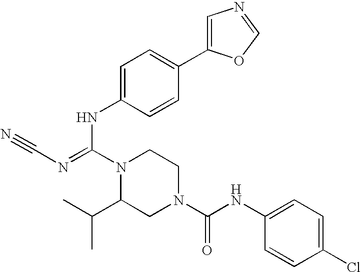 | 2.31 (k) | 492 (M + H)⁺ |
| 4.70 | 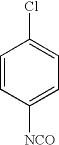 | C | 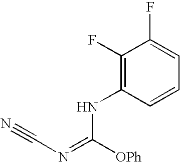 | B | 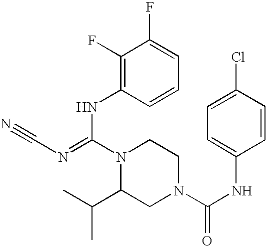 | 2.45 (k) | 461 (M + H)⁺ |
| 4.71 | 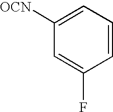 | C | 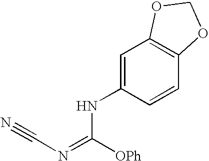 | B | 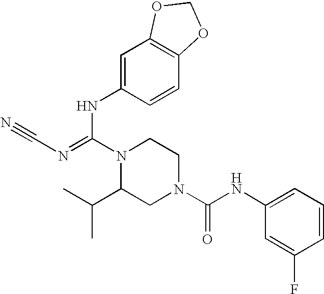 | 2.26 (k) | 453 (M + H)⁺ |
| 4.72 | 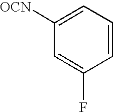 | C | 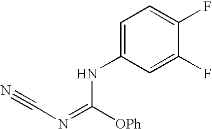 | B | 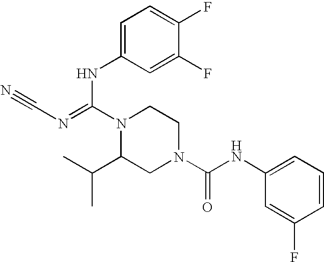 | 2.38 (k) | 445 (M + H)⁺ |
| 4.73 | 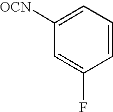 | C | 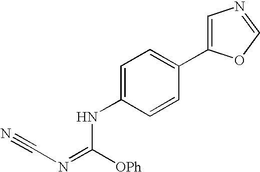 | B | 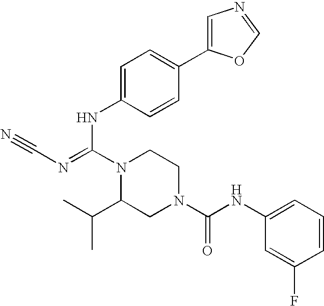 | 2.19 (k) | 476 (M + H)⁺ |
| 4.74 | 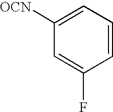 | C | 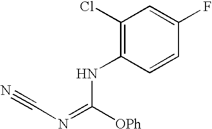 | B | 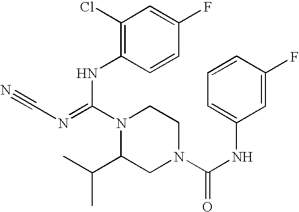 | 2.37 (k) | 461 (M + H)⁺ |
| 4.75 | 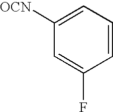 | C | 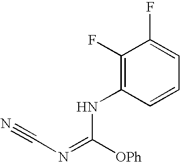 | B | 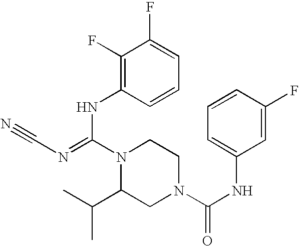 | 2.34 (k) | 445 (M + H)⁺ |
| 4.76 | 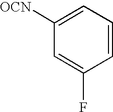 | C | 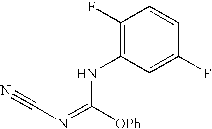 | B | 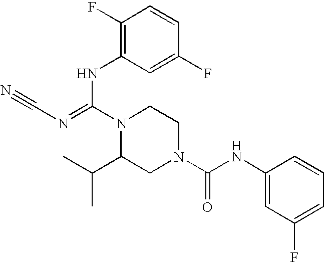 | 2.38 (k) | 445 (M + H)⁺ |
| 4.77 | 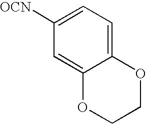 | C | 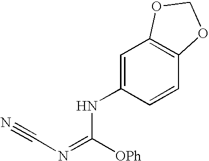 | B | 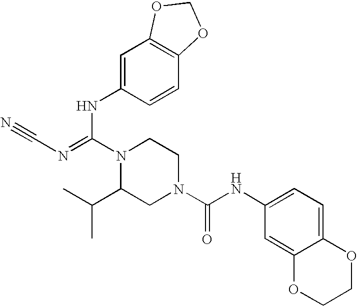 | 2.07 (k) | 493 (M + H)⁺ |
| 4.78 | 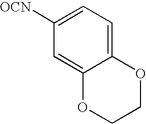 | C | 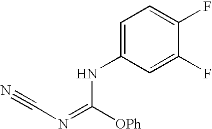 | B | 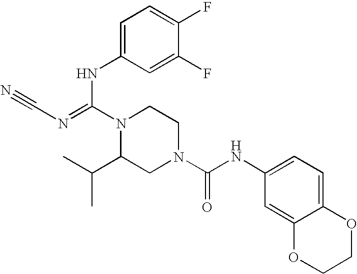 | 2.12 (k) | 485 (M + H)⁺ |
| 4.79 | 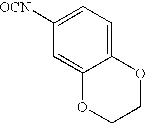 | C | 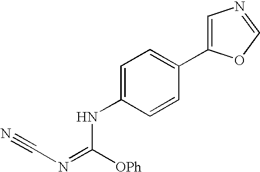 | B | 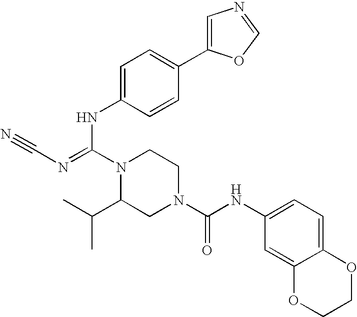 | 2.02 (k) | 516 (M + H)⁺ |
| 4.80 | 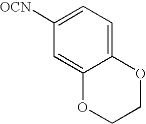 | C | 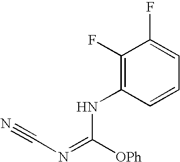 | B | 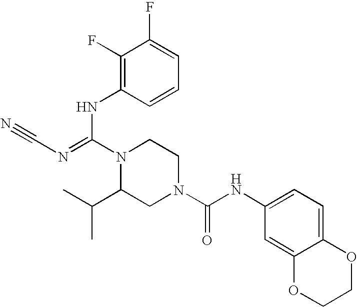 | 2.16 (k) | 485 (M + H)⁺ |
| 4.81 | 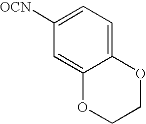 | C | 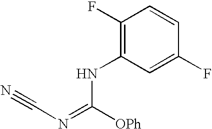 | B | 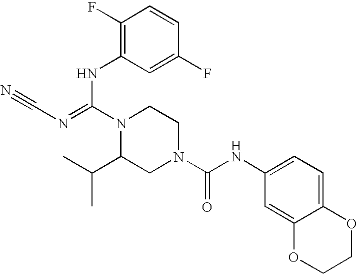 | 2.2 (k) | 485 (M + H)⁺ |
| 4.82 | 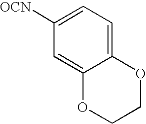 | C | 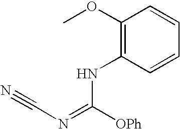 | B | 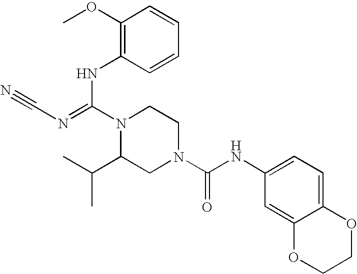 | 2.3 (k) | 479 (M + H)⁺ |
| 4.83 | 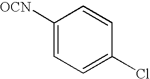 | C | 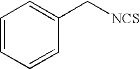 | G | 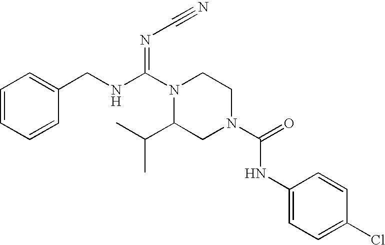 | 1.93 (d) | 437 (M − H)⁻ |
| 4.84 | 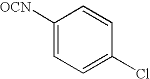 | C | 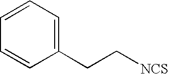 | G | 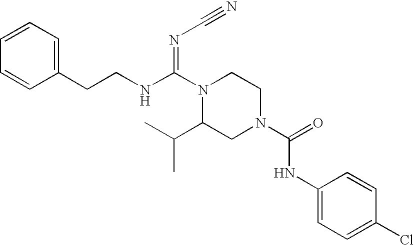 | 2.01 (d) | 451 (M − H)⁻ |
| 4.85 | 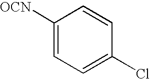 | C | 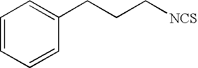 | G | 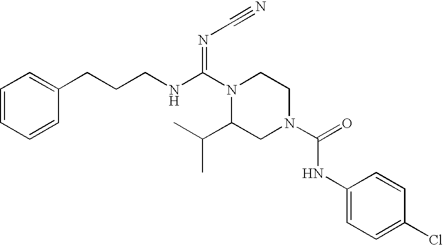 | 2.10 (d) | 465 (M − H)⁻ |
| 4.86 | 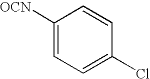 | C | 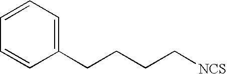 | G | 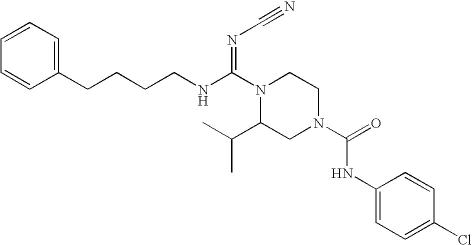 | 2.21 (d) | 479 (M − H)⁻ |
| 4.87 | 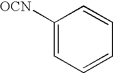 | C | 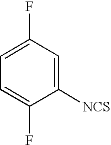 | G | 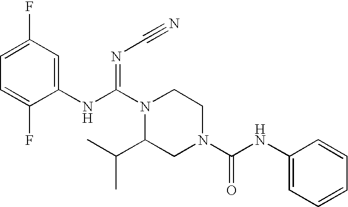 | 1.71 (d) | 425 (M − H)⁻ |
| 4.88 | 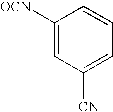 | C | 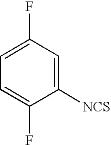 | G | 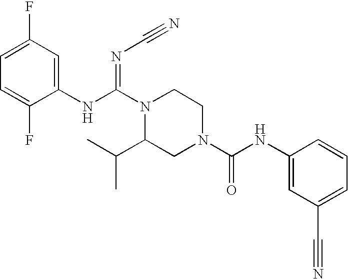 | 1.76 (d) | 450 (M − H)⁻ |
| 4.89 | 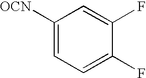 | C | 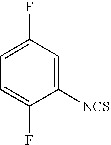 | G | 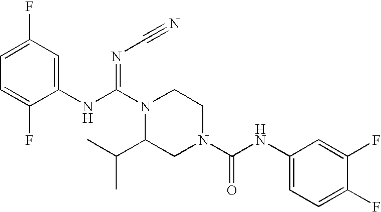 | 1.87 (d) | 461 (M − H)⁻ |
| 4.90 | 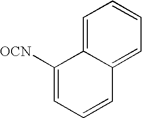 | C | 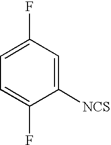 | G | 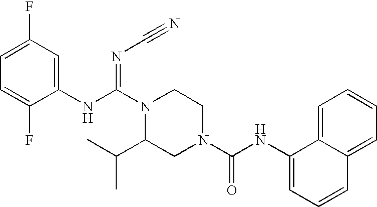 | 1.83 (d) | 475 (M − H)⁻ |
| 4.91 | 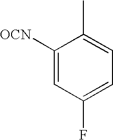 | C | 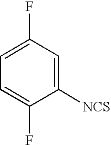 | G | 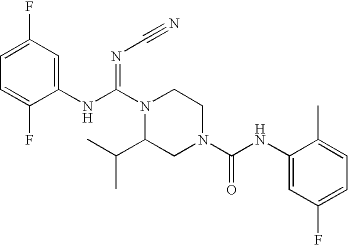 | 1.82 (d) | 457 (M − H)⁻ |
| 4.92 | 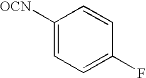 | C | 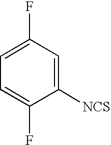 | G | 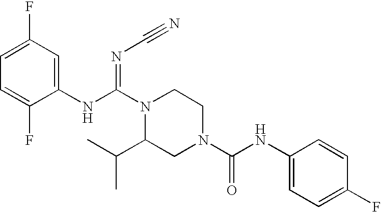 | 1.75 (d) | 443 (M − H)⁻ |
| 4.93 | 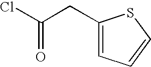 | D | 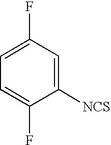 | G | 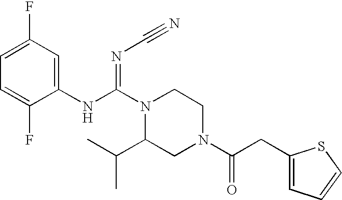 | 1.71 (d) | 430 (M − H)⁻ |
| 4.94 | 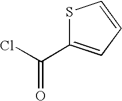 | D | 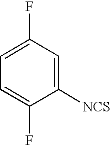 | G | 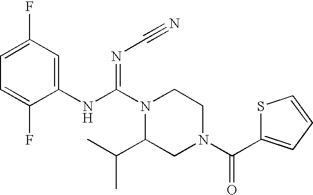 | 1.66 (d) | 416 (M − H)⁻ |
| 4.95 | 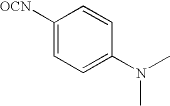 | C | 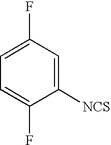 | G | 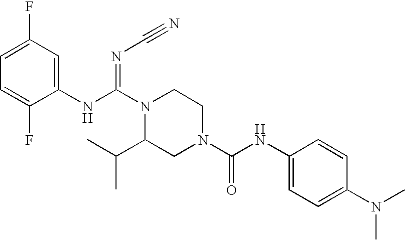 | 1.71 (d) | 468 (M − H)⁻ |
| 4.96 | 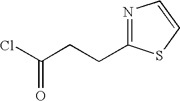 | D | 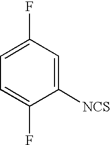 | G | 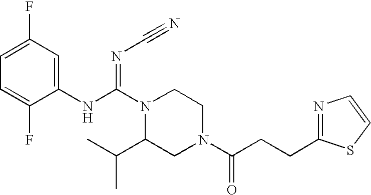 | 1.44 (d) | 445 (M − H)⁻ |
| 4.97 | 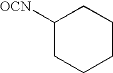 | C | 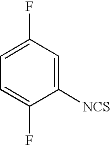 | G | 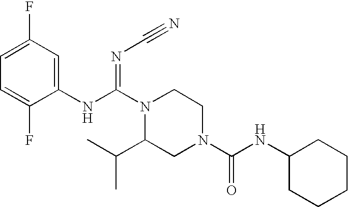 | 1.77 (d) | 431 (M − H)⁻ |
| 4.98 | 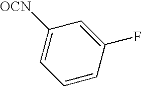 | C | 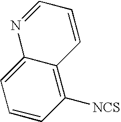 | G | 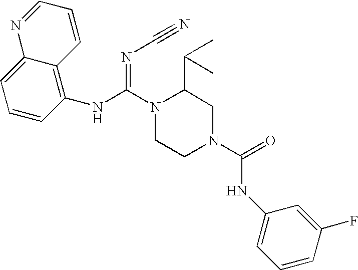 | 1.56 (d) | 460 (M + H)⁺ |
| 4.99 | 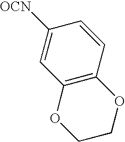 | C | 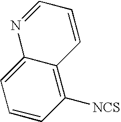 | G | 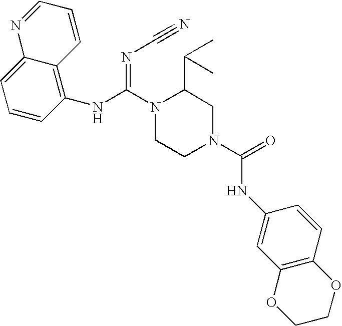 | 1.36 (d) | 500 (M + H)⁺ |
| 4.100 | 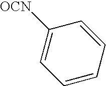 | C | 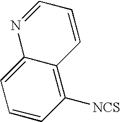 | G | 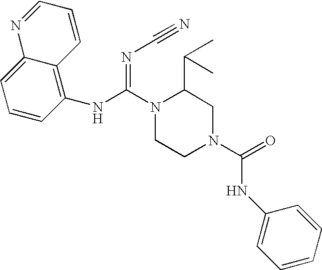 | 1.48 (d) | 442 (M + H)⁺ |
| 4.101 | 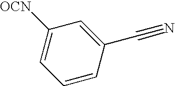 | C | 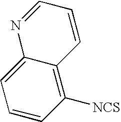 | G | 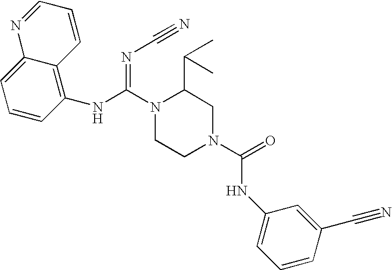 | 1.50 (d) | 467 (M + H)⁺ |
| 4.102 | 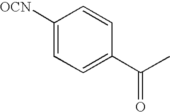 | C | 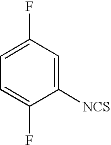 | G | 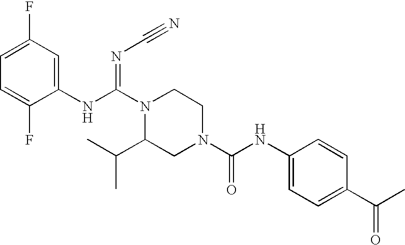 | 1.60 (d) | 467 (M − H)⁻ |
| 4.103 | 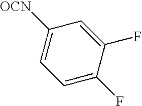 | C | 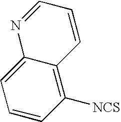 | G | 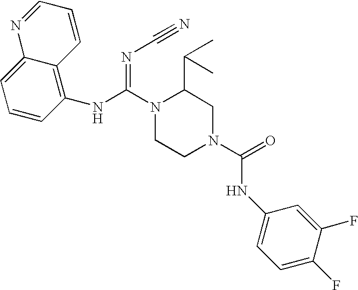 | 1.68 (d) | 478 (M + H)⁺ |
| 4.104 | 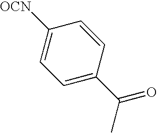 | C | 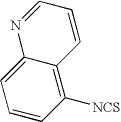 | G | 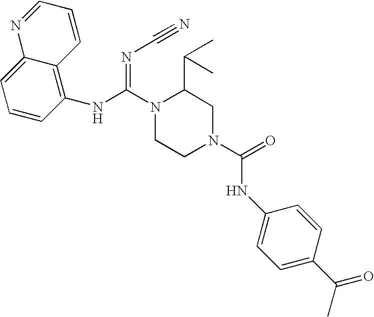 | 1.38 (d) | 484 (M + H)⁺ |
| 4.105 | 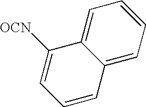 | C | 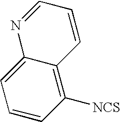 | G | 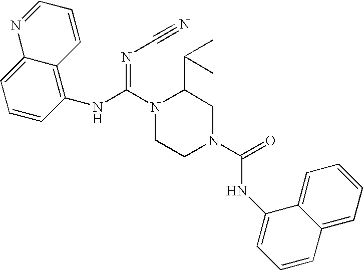 | 1.57 (d) | 492 (M + H)⁺ |
| 4.106 | 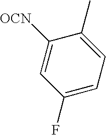 | C | 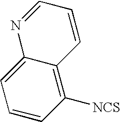 | G | 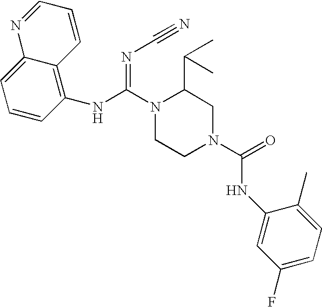 | 1.58 (d) | 474 (M + H)⁺ |
| 4.107 | 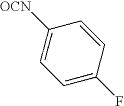 | C | 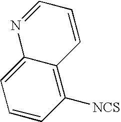 | G | 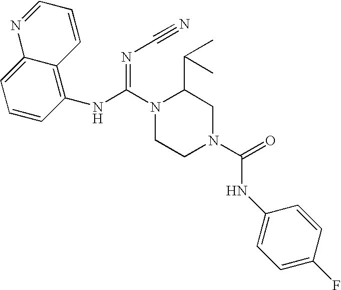 | 1.51 (d) | 460 (M + H)⁺ |
| 4.108 | 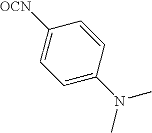 | C | 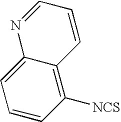 | G | 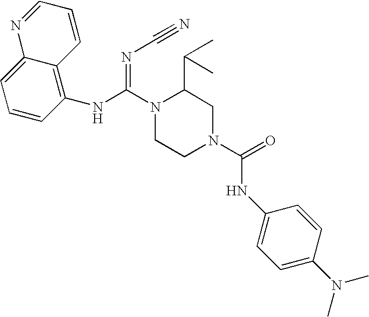 | 1.42 (d) | 485 (M + H)⁺ |
| 4.109 | 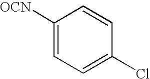 | C | 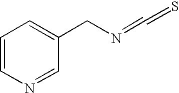 | G | 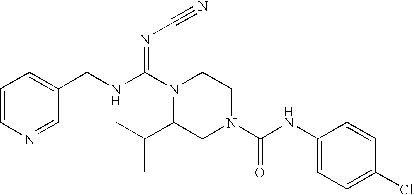 | 1.37 (d) | 440 (M + H)⁺ |
| 4.110 | 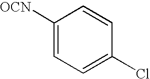 | C | 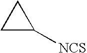 | G | 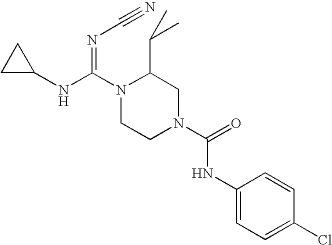 | 1.72 (d) | 389 (M + H)⁺ |
| 4.111 | 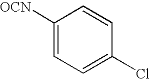 | C | 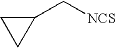 | G | 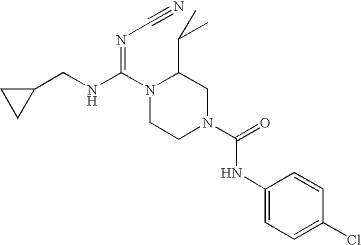 | 1.86 (d) | 403 (M + H)⁺ |
| 4.112 | 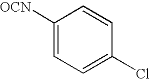 | C | 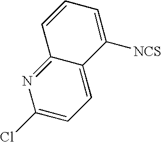 | G | 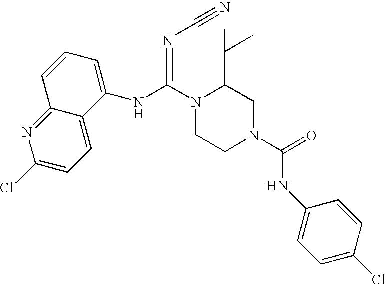 | 2.00 (d) | 510 (M + H)⁺ |
| 4.113 | 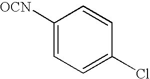 | C | 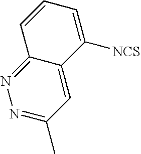 | G | 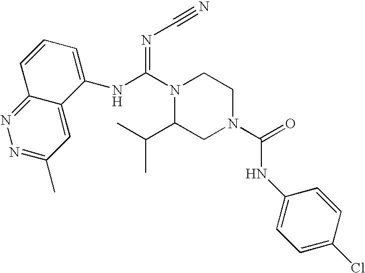 | 1.63 (d) | 491 (M + H)⁺ |
| 4.114 | 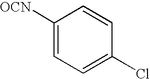 | C | 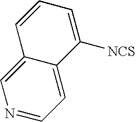 | G | 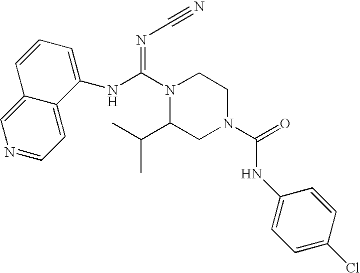 | 1.65 (d) | 476 (M + H)⁺ |
| 4.115 | 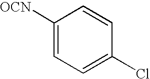 | C | 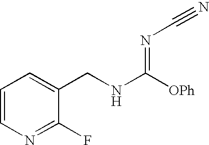 | B | 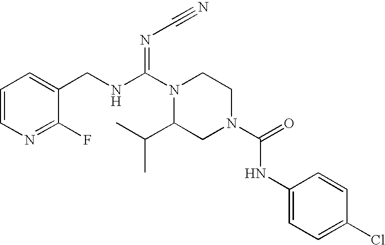 | 1.32 (d) | 458 (M + H)⁺ |
| 4.116 | 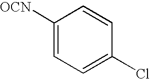 | C | 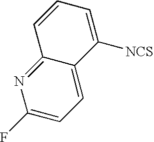 | G | 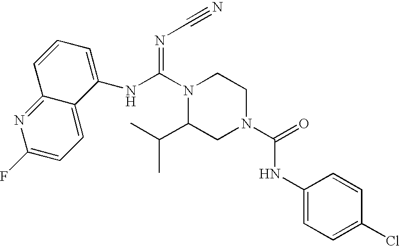 | 1.83 (d) | 494 (M + H)⁺ |
| 4.117 | 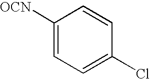 | C | 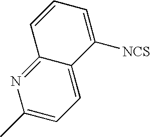 | G | 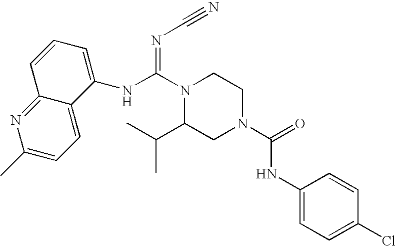 | 1.70 (d) | 490 (M + H)⁺ |
| 4.118 | 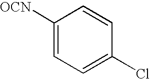 | C | 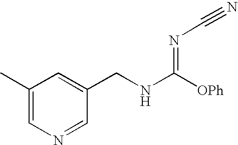 | B | 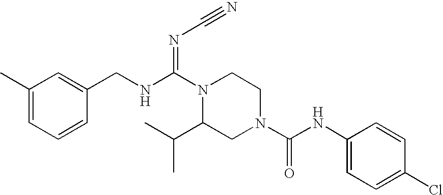 | 1.17 (d) | 454 (M + H)⁺ |
| 4.119 | 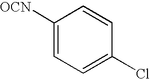 | C | 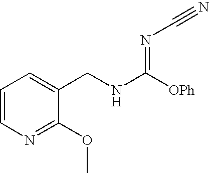 | B | 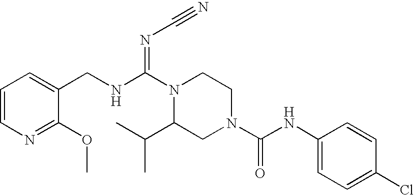 | 1.65 (d) | 470 (M + H)⁺ |
| 4.120 | 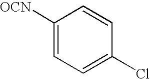 | C | 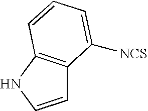 | G | 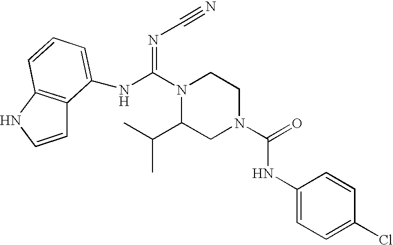 | 1.80 (d) | 464 (M + H)⁺ |
| 4.121 | 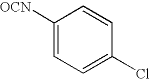 | C | 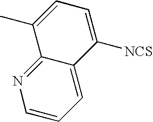 | G | 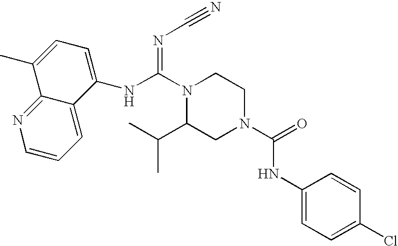 | 1.78 (d) | 490 (M + H)⁺ |
| 4.122 | 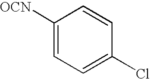 | C | 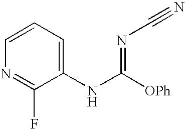 | B | 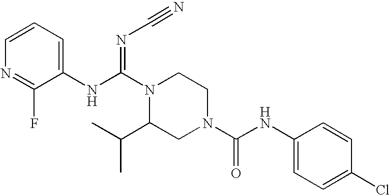 | 1.71 (d) | 444 (M + H)⁺ |
| 4.123 | 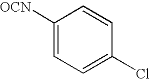 | C | 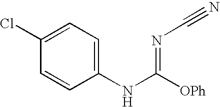 | B | 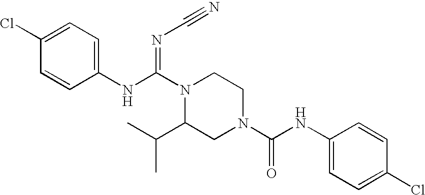 | 1.99 (d) | 459 (M + H)⁺ |
| 4.124 | 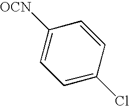 | C | 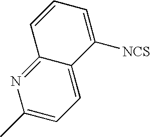 | G | 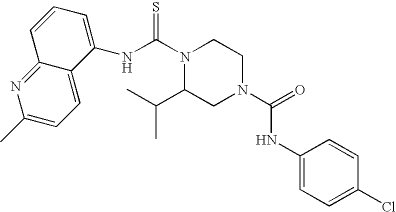 | 1.93 (d) | 480 (M + H)⁺ |
| 4.125 | 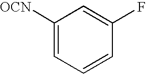 | C | 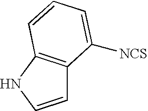 | G | 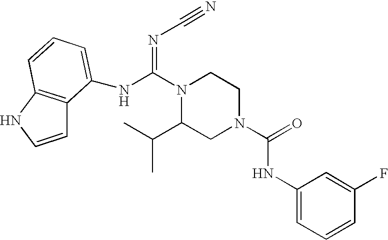 | 1.76 (d) | 448 (M + H)⁺ |
| 4.126 | 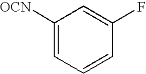 | C | 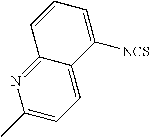 | G | 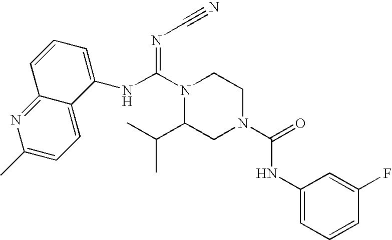 | 1.66 (d) | 474 (M + H)⁺ |
| 4.127 | 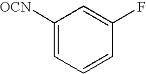 | C | 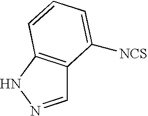 | G | 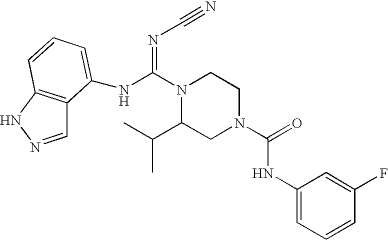 | 1.58 (d) | 449 (M + H)⁺ |
| 4.128 | 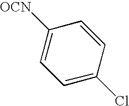 | C | 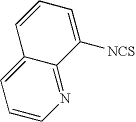 | G | 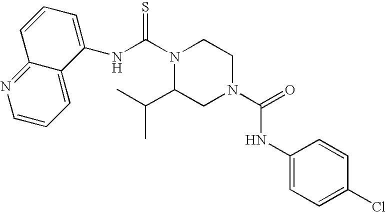 | 1.89 (d) | 468 (M + H)⁺ |
| 4.129 | 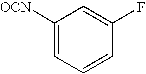 | C | 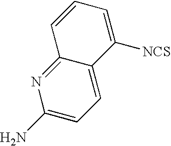 | G | 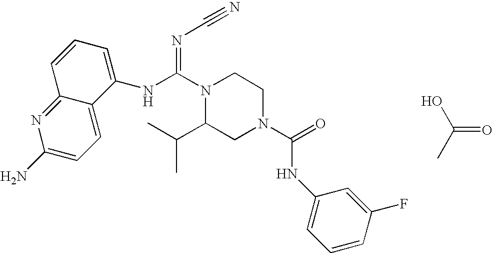 | 1.48 (d) | 475 (M + H)⁺ |
| 4.130 | 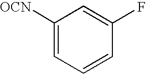 | C | 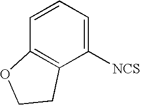 | G | 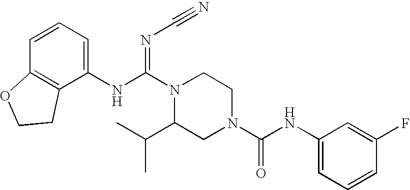 | 1.82 (d) | 449 (M − H)⁻ |
| 4.131 | 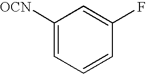 | C | 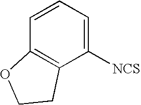 | G | 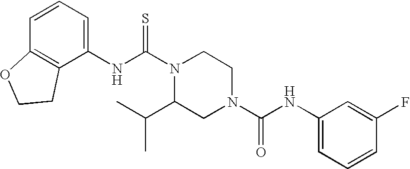 | 2.05 (d) | 441 (M − H)⁻ |
| 4.132 | 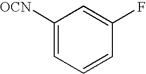 | C | 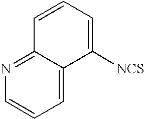 | G | 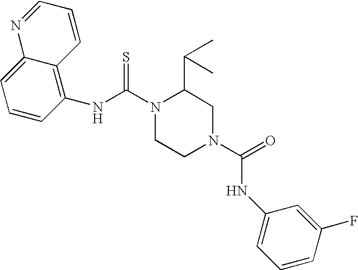 | 1.79 (d) | 450 (M − H)⁻ |
| 4.133 | 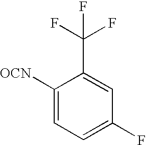 | C | 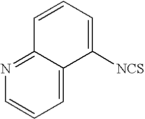 | G | 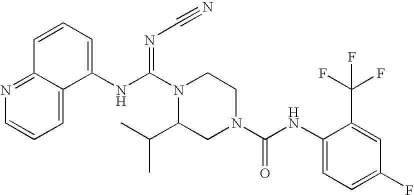 | 1.84 (d) | 528 (M + H)⁺ |
| 4.134 | 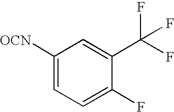 | C | 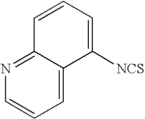 | G | 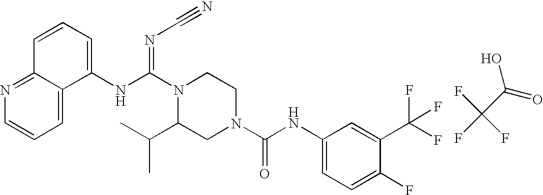 | 1.95 (d) | 528 (M + H)⁺ |
| 4.135 | 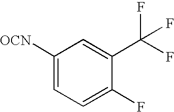 | C | 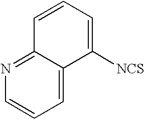 | G | 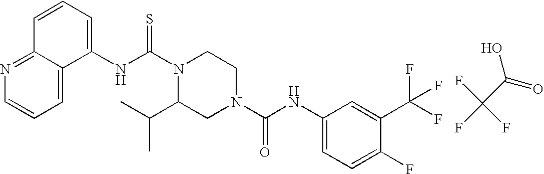 | 2.12 (d) | 518 (M − H)⁻ |
| 4.136 | 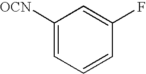 | C | 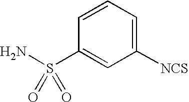 | G | 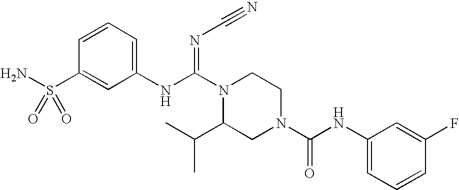 | 1.61 (d) | 486 (M − H)⁻ |
| 4.137 | 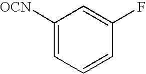 | C | 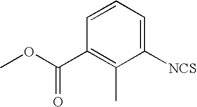 | G | 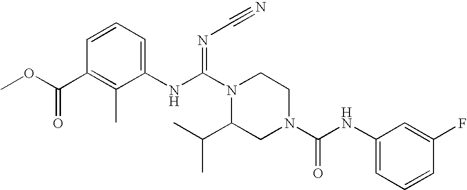 | 1.85 (d) | 479 (M − H)⁻ |
| 4.138 | 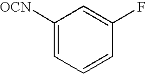 | C | 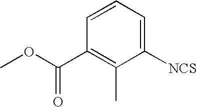 | G | 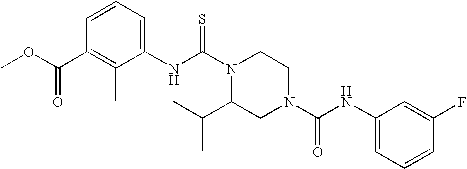 | 2.08 (d) | 471 (M − H)⁻ |
| 4.139 | 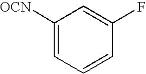 | C | 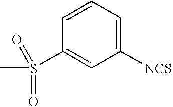 | G | 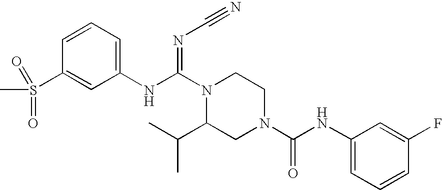 | 1.66 (d) | 485 (M − H)⁻ |
| 4.140 | 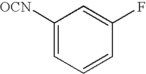 | C | 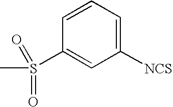 | G | 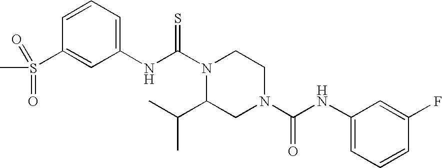 | 1.66 (d) | 485 (M − H)⁻ |
| 4.141 | 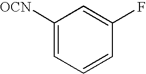 | C | 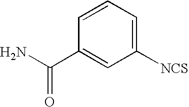 | G | 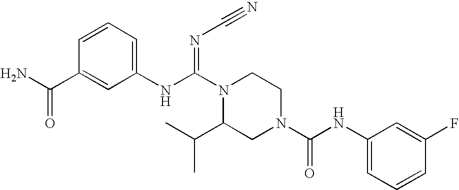 | 1.45 (d) | 450 (M − H)⁻ |
| 4.142 | 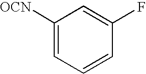 | C | 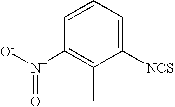 | G | 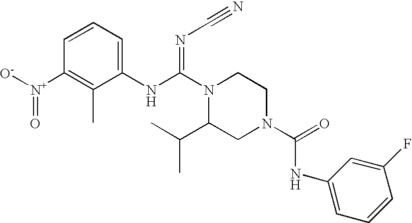 | 1.84 (d) | 468 (M + H)⁺ |
| 4.143 | 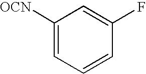 | C | 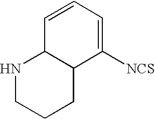 | G | 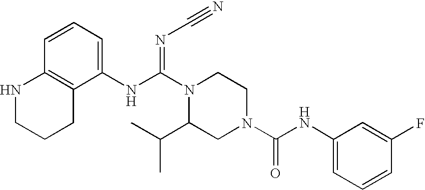 | 1.74 (d) | 462 (M − H)⁻ |
| 4.144 | 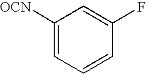 | C | 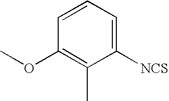 | G | 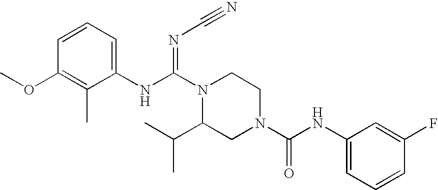 | 1.88 (d) | 451 (M − H)⁻ |
| 4.145 | 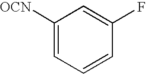 | C | 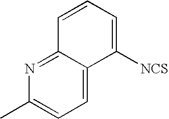 | G | 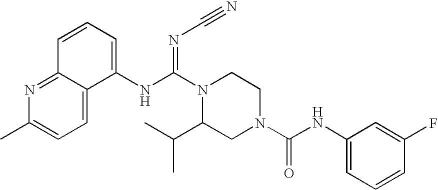 | 1.87 (b) | 474 (M + H)⁺ |
| 4.146 | 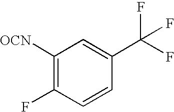 | C | 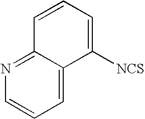 | G | 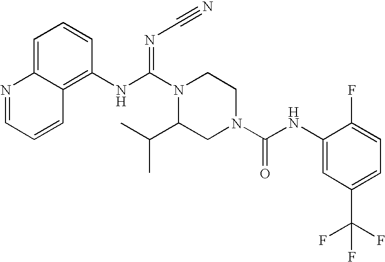 | 2.69 (a) | 528 (M + H)⁺ |
| 4.147 | 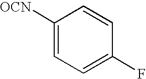 | C | 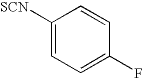 | G | 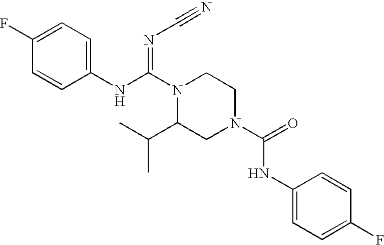 | 2.40 (a) | 427 (M + H)⁺ |
| 4.148 | 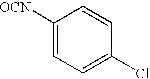 | C | 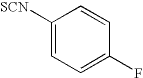 | G | 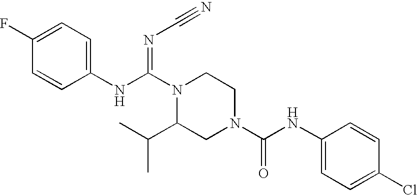 | 2.60 (a) | 443 (M + H)⁺ |
| 4.149 | 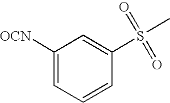 | C | 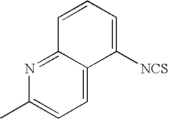 | G | 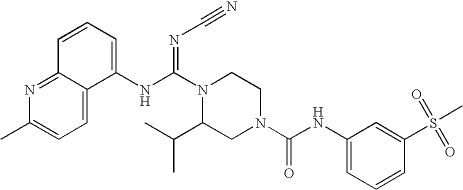 | 1.89 (a) | 534 (M + H)⁺ |
| 4.150 | 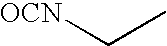 | C | 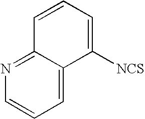 | G | 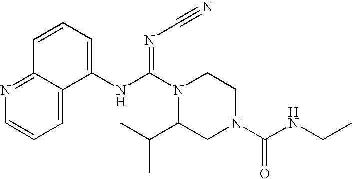 | 1.66 (a) | 394 (M + H)⁺ |
| 4.151 | 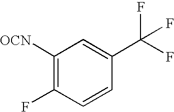 | C | 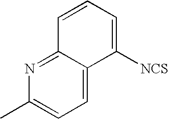 | G | 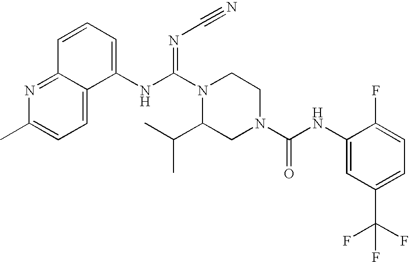 | 2.46 (a) | 542 (M + H)⁺ |
| 4.152 | 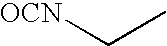 | C | 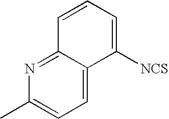 | G | 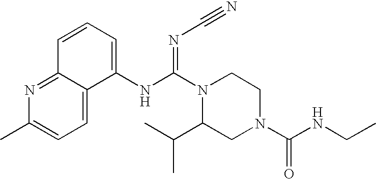 | 1.65 (a) | 408 (M + H)⁺ |
| 4.153 | 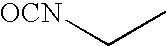 | C | 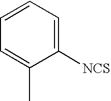 | G | 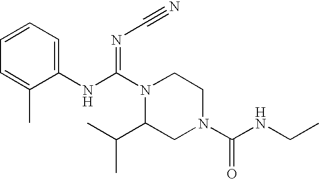 | 1.88 (a) | 409 (M + H)⁺ |
| 4.154 | 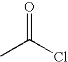 | D | 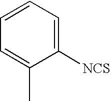 | G | 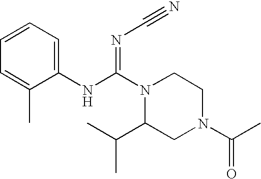 | 1.58 (b) | 328 (M + H)⁺ |
| 4.155 | 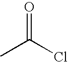 | D | 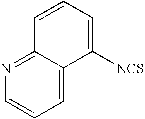 | G | 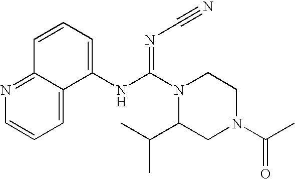 | 1.63 (a) | 365 (M + H)⁺ |
| 4.156 | 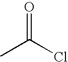 | D | 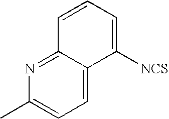 | G | 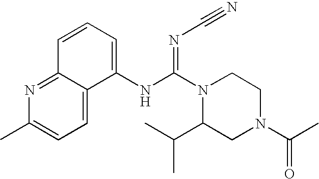 | 1.63 | 379 (M + H)⁺ |
| 4.157 | 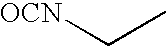 | D | 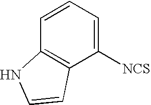 | G | 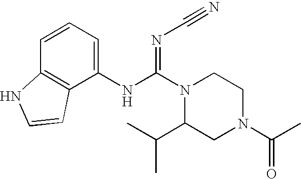 | 1.79 (a) | 353 (M + H)⁺ |
| 4.158 | 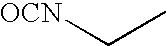 | C | 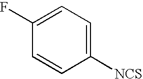 | G | 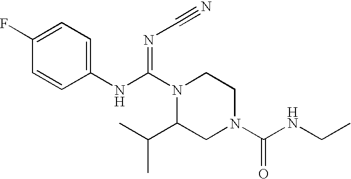 | 1.99 (a) | 361 (M + H)⁺ |
| 4.159 | 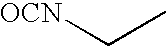 | C | 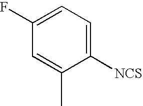 | G | 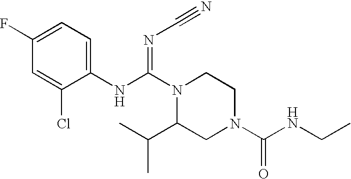 | 1.44 (d) | 395 (M + H)⁺ |

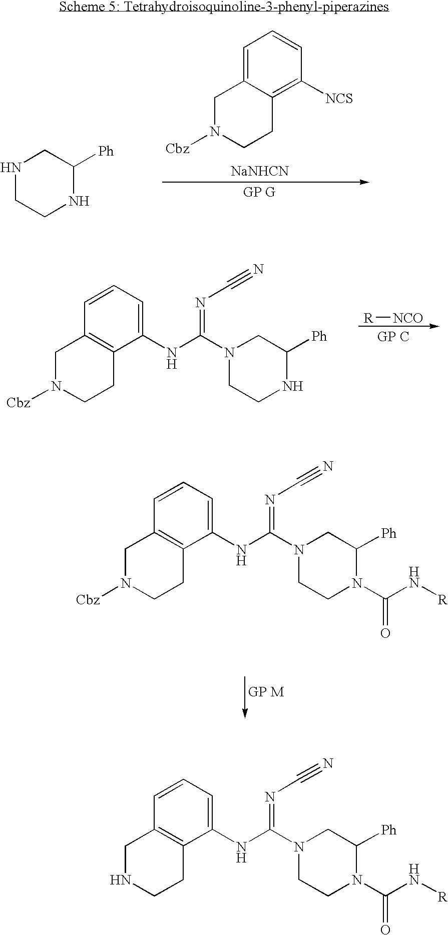

The compounds in table 5 were made according to general procedure G, followed by general procedures C and M as illustrated in scheme 5.

TABLE 5
Tetrahydroisoquinoline-3-phenyl-piperazines

| | | | Rt/min | |
|---|---|---|---|---|
| Ex # | R-NCO | Product | (method) | m/z |
| 5.1 | 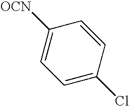 | 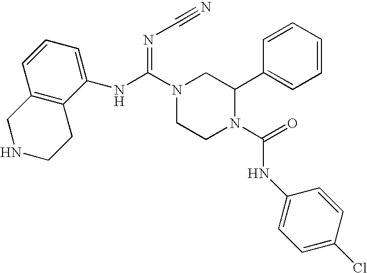 | 1.91 (d) | 514 (M + H)⁺ |
| 5.2 | 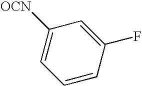 | 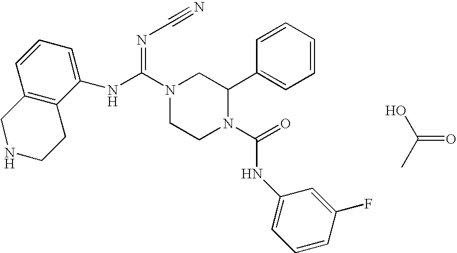 | 1.47 (d) | 498 (M + H)⁺ |
| 5.3 | 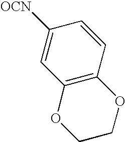 | 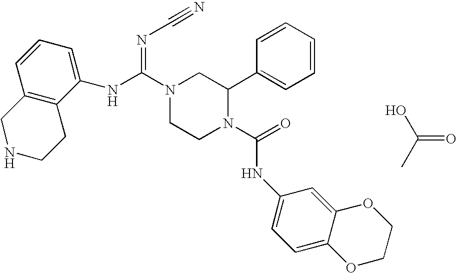 | 1.29 (d) | 538 (M + H)⁺ |
| 5.4 | 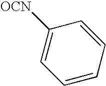 | 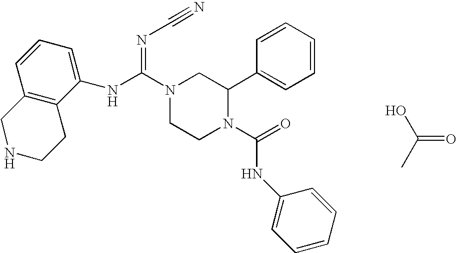 | 1.36 (d) | 480 (M + H)⁺ |
| 5.5 | 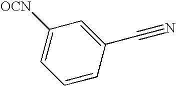 | 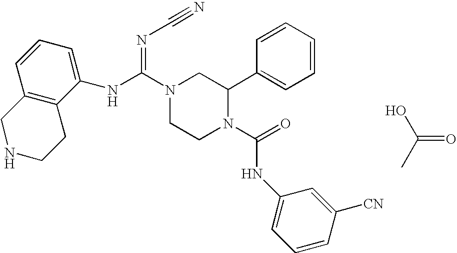 | 1.44 (d) | 505 (M + H)⁺ |
| 5.6 | 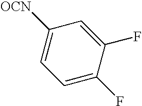 | 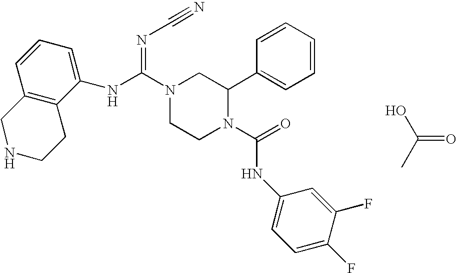 | 1.59 (d) | 516 (M + H)⁺ |
| 5.7 | 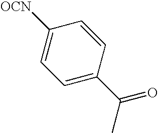 | 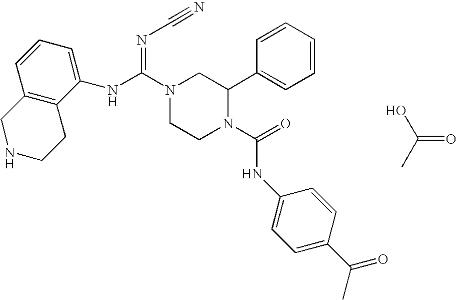 | 1.25 (d) | 522 (M + H)⁺ |
| 5.8 | 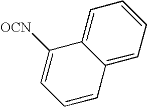 | 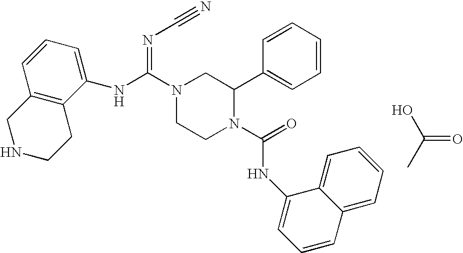 | 1.61 (d) | 530 (M + H)⁺ |
| 5.9 | 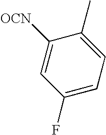 | 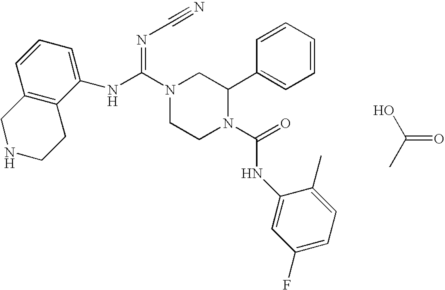 | 1.61 (d) | 512 (M + H)⁺ |
| 5.10 | 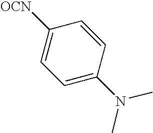 | 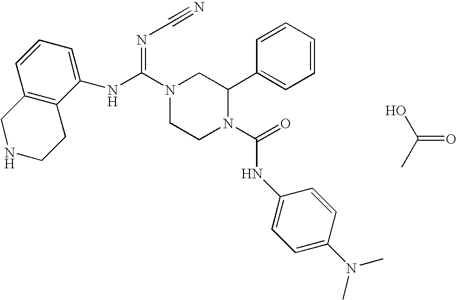 | 1.41 (d) | 523 (M + H)⁺ |
| 5.11 | 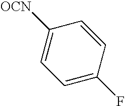 | 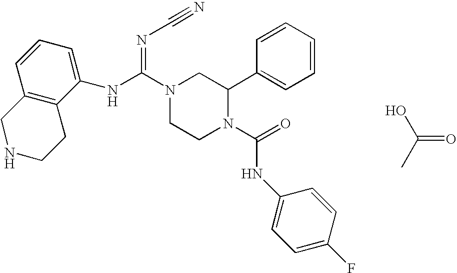 | 1.35 (d) | 598 (M + H)⁺ |

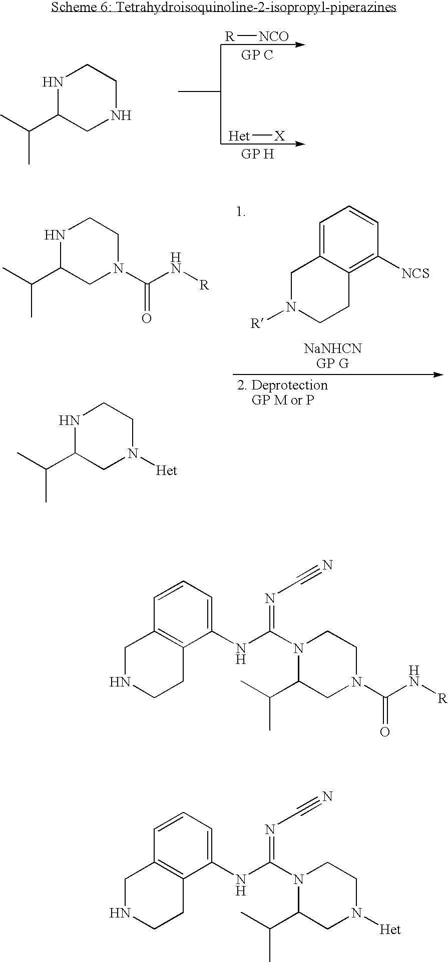

The compounds in table 6 were made according to either general procedure C or H, followed by general procedure G and either general procedure M or P as illustrated in scheme 6. The specific procedures used for each compound are listed in table 6.

TABLE 6
Tetrahydroisoquinoline-2-isopropyl-piperazines

| | R-NCO or | | | Deprotection | | Rt/min | |
|---|---|---|---|---|---|---|---|
| Ex # | Het-X | GP | R′ | GP | Product | (method) | m/z |
| 6.1 | 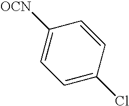 | C | Cbz | M | 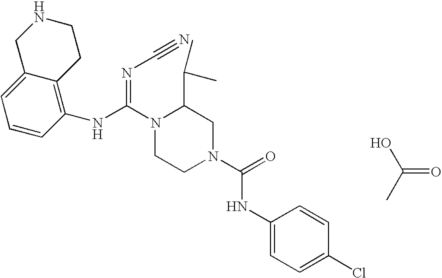 | 1.65 (d) | 480 (M + H)⁺ |
| 6.2 | 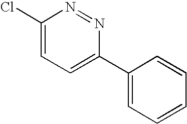 | H | Cbz | M | 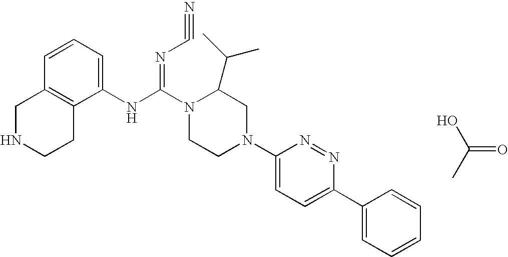 | 1.60 (d) | 479 (M + H)⁺ |
| 6.3 | 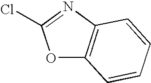 | H | Cbz | M | 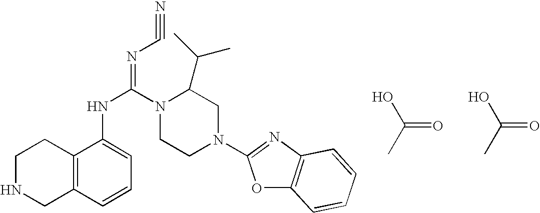 | 1.47 (d) | 444 (M + H)⁺ |
| 6.4 | 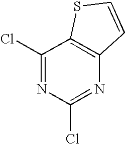 | H | Fmoc | P | 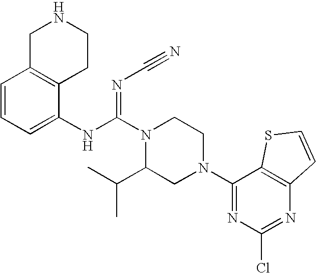 | 1.46 (d) | 493 (M − H)⁻ |
| 6.5 | 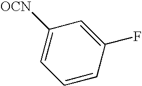 | C | Cbz | M | 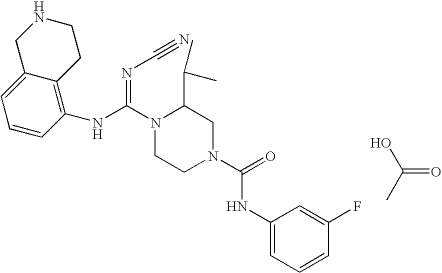 | 1.39 (d) | 464 (M + H)⁺ |
| 6.6 | 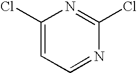 | H | Fmoc | P | 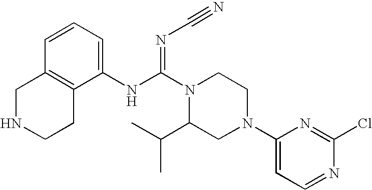 | 1.33 (d) | 437 (M − H)⁻ |
| 6.7 | 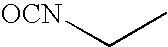 | C | Cbz | M | 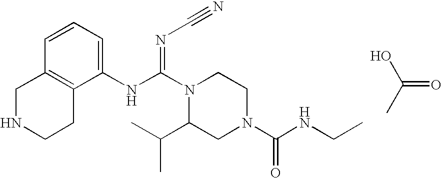 | 1.53 (d) | 378 (M + H)⁺ |

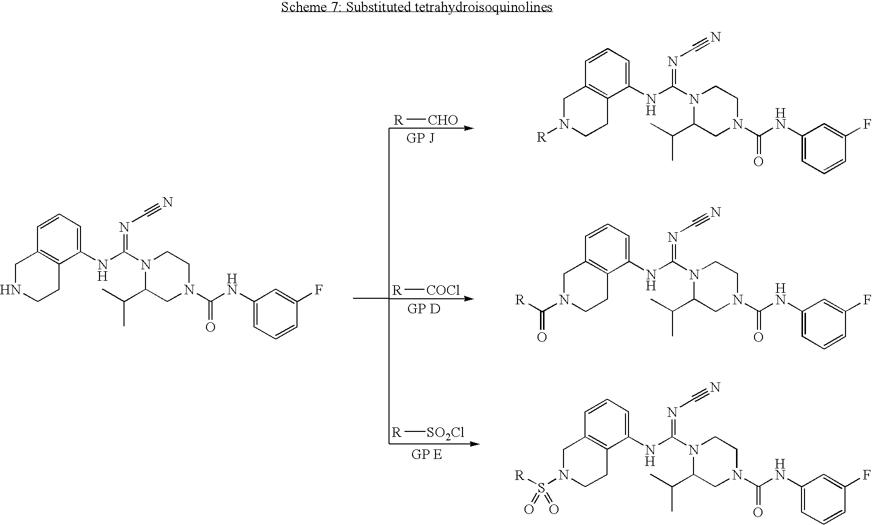

The compounds in table 7 were made according to either general procedure D or E or J. as illustrated in scheme 7. The specific procedure used for each compound is listed in table 7.

TABLE 7
Substituted tetrahydroisoquinolines

| | | | | Rt/min | |
|---|---|---|---|---|---|
| Ex # | Reagent | GP | Product | (method) | m/z |
| 7.1 | 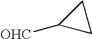 | J | 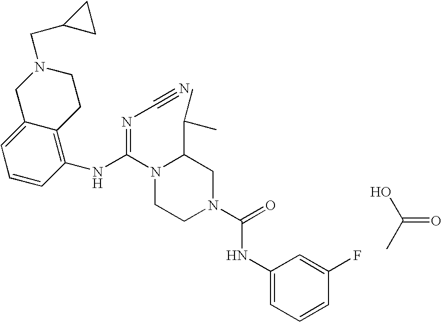 | 1.63 (d) | 518 (M + H)⁺ |
| 7.2 | 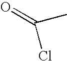 | D | 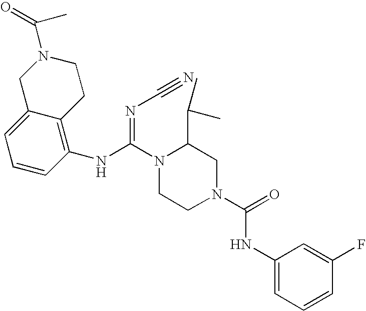 | 1.55 (d) | 506 (M + H)⁺ |
| 7.3 | 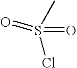 | E | 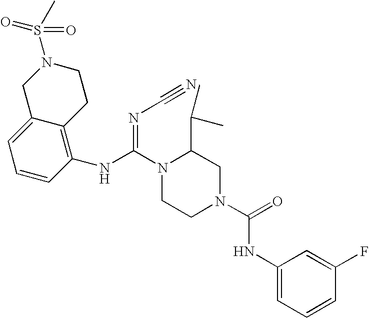 | 1.73 (d) | 542 (M + H)⁺ |
| 7.4 | 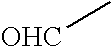 | J | 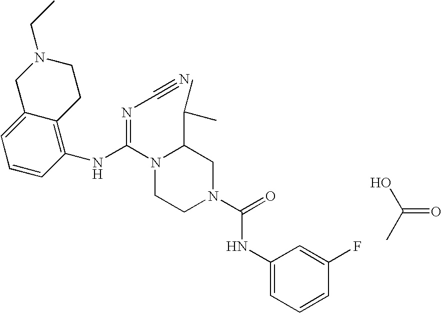 | 1.56 (d) | 492 (M + H)⁺ |
| 7.5 | 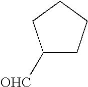 | J | 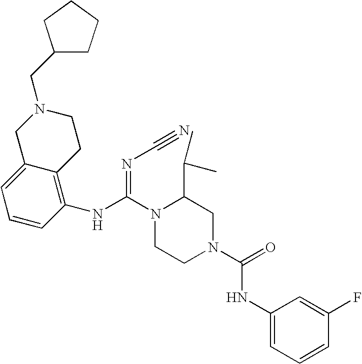 | 1.96 (d) | 546 (M + H)⁺ |
| 7.6 | 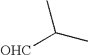 | J | 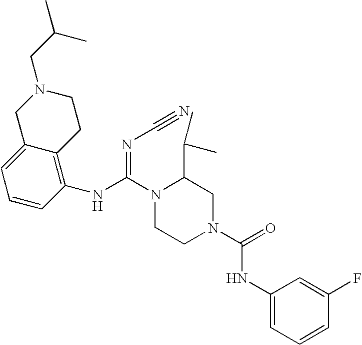 | 1.74 (d) | 520 (M + H)⁺ |
| 7.7 | 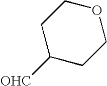 | J | 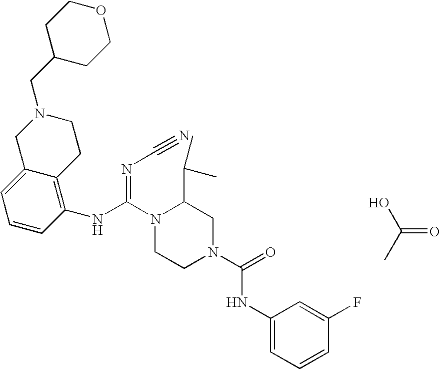 | 1.54 (d) | 562 (M + H)⁺ |
| 7.8 | 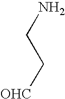 | J | 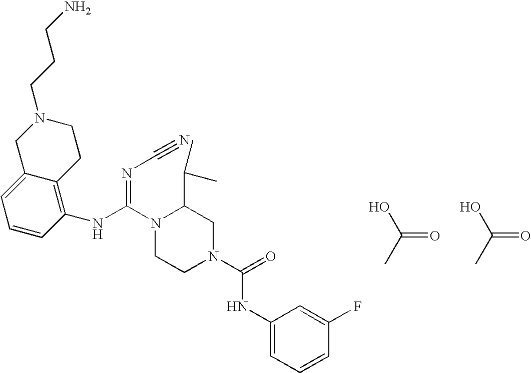 | 1.16 (d) | 521 (M + H)⁺ |
| 7.9 | 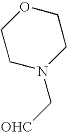 | J | 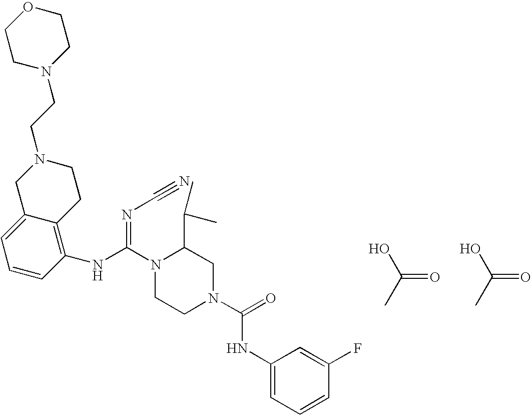 | 1.47 (d) | 577 (M + H)⁺ |
| 7.10 | 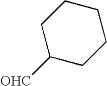 | J | 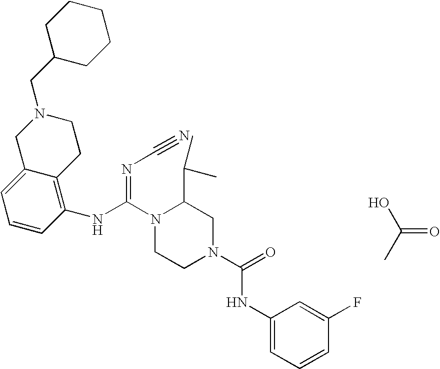 | 1.95 (d) | 560 (M + H)⁺ |
| 7.11 | 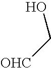 | J | 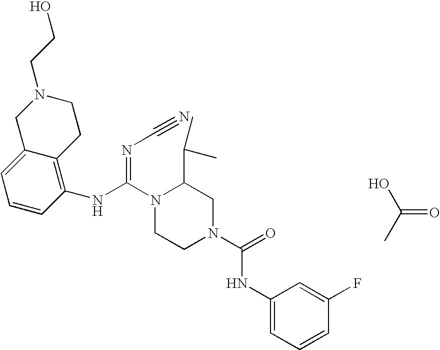 | 1.35 (d) | 508 (M + H)⁺ |
| 7.12 | 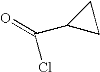 | D | 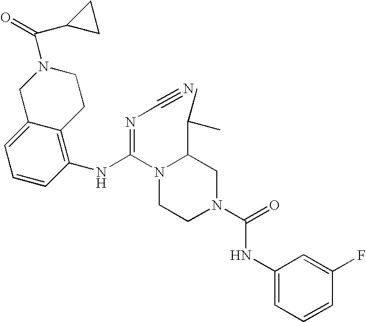 | 1.68 (d) | 532 (M + H)⁺ |
| 7.13 | 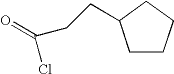 | D | 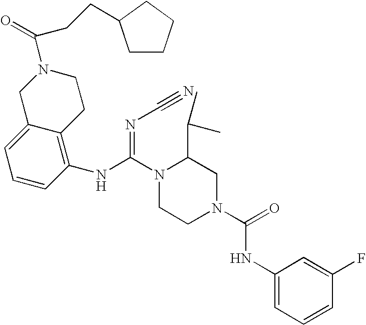 | 2.09 (d) | 588 (M + H)⁺ |
| 7.14 | 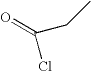 | D | 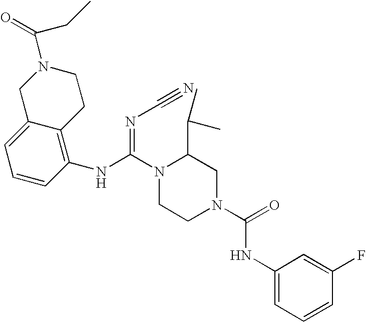 | 1.62 (d) | 520 (M + H)⁺ |
| 7.15 | 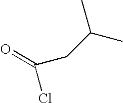 | D | 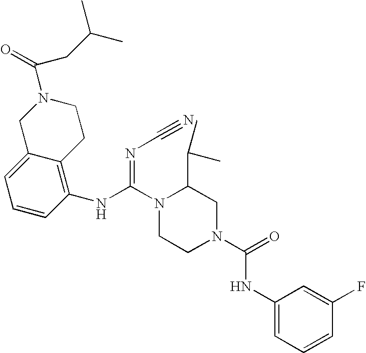 | 1.82 (d) | 548 (M + H)⁺ |
| 7.16 | 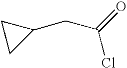 | D | 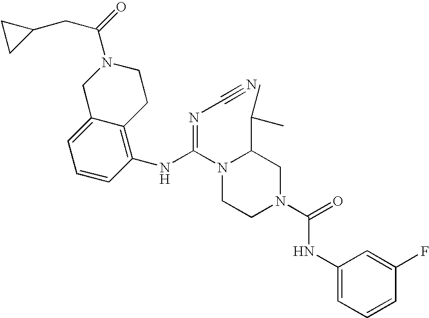 | 1.75 (d) | 546 (M + H)⁺ |
| 7.17 | 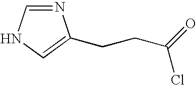 | D | 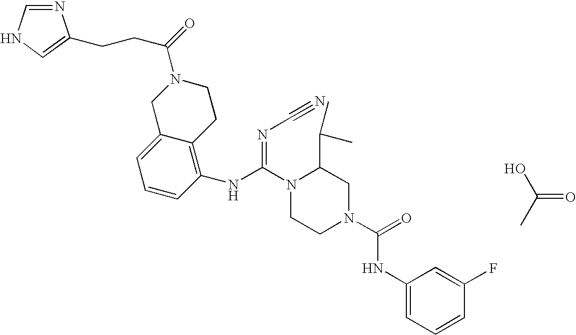 | 1.41 (d) | 586 (M + H)⁺ |
| 7.18 | 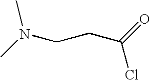 | D | 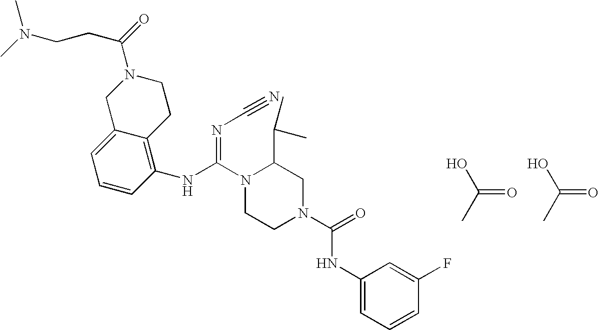 | 1.44 (d) | 563 (M + H)⁺ |
| 7.19 | 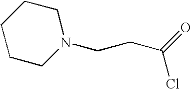 | D | 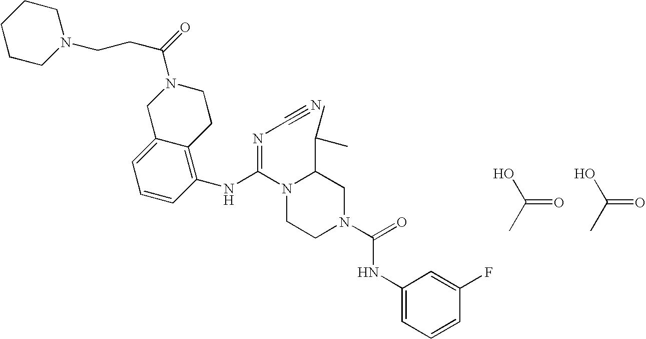 | 1.55 (d) | 603 (M + H)⁺ |
| 7.20 | 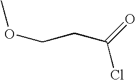 | D | 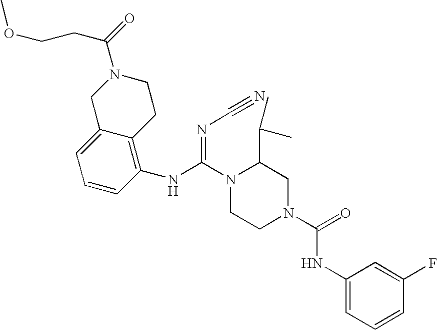 | 1.61 (d) | 550 (M + H)⁺ |
| 7.21 | 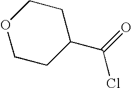 | D | 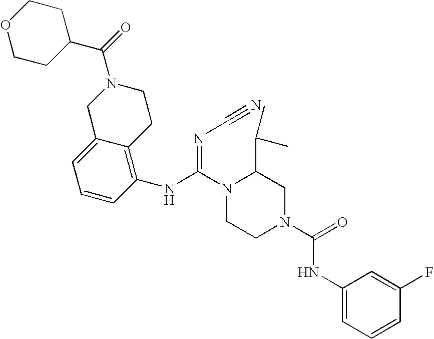 | 1.59 (d) | 576 (M + H)⁺ |
| 7.22 | 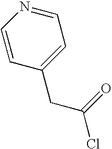 | D | 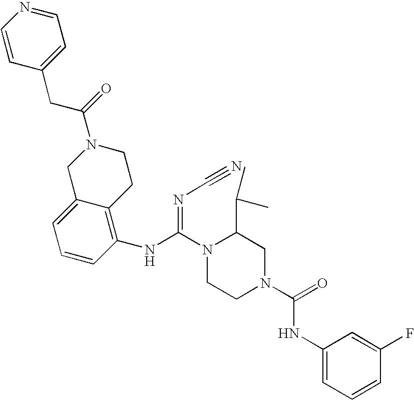 | 1.56 (d) | 583 (M + H)⁺ |
| 7.23 | 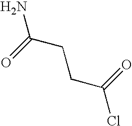 | D | 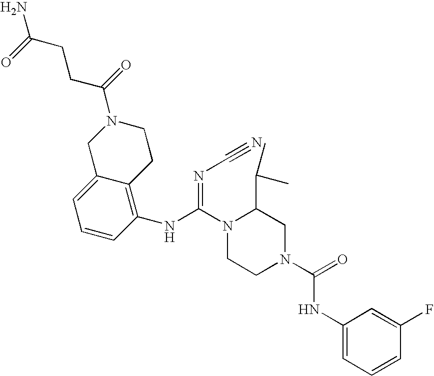 | 1.41 (d) | 563 (M + H)⁺ |

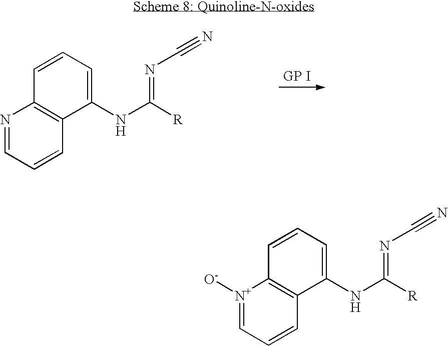

The compounds in table 8 were made according to general procedure I, as illustrated in scheme 8.

TABLE 8
Quinoline-N-oxides

| | | | | Rt/min |
|---|---|---|---|---|
| Ex # | Reactant | Product | (method) | m/z |
| 8.1 | 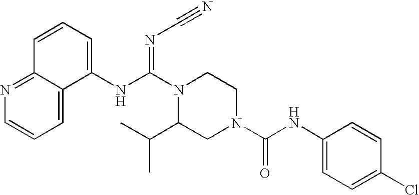 | 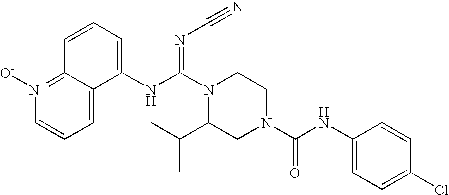 | 1.50 (d) | 490 (M − H)⁻ |
| 8.2 | 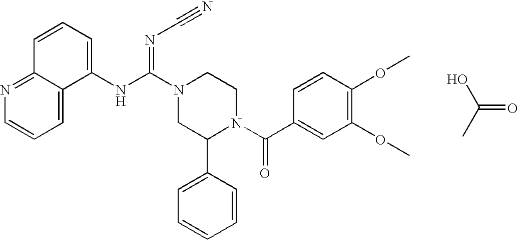 | 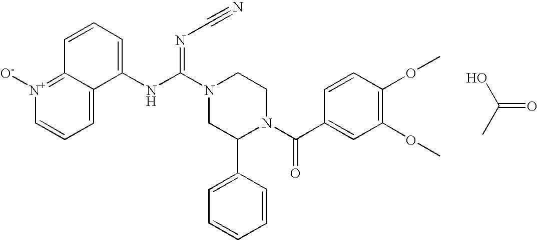 | 1.22 (d) | 435 (M − H)⁻ |
| 8.3 | 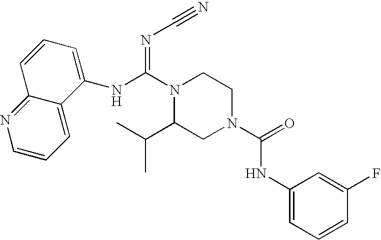 | 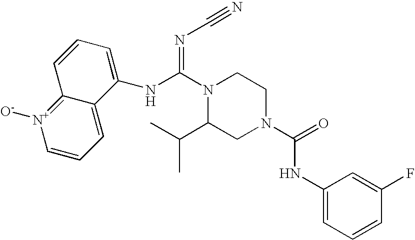 | 1.38 (d) | 476 (M + H)⁺ |
| 8.4 | 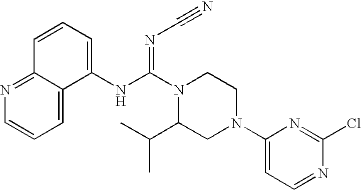 | 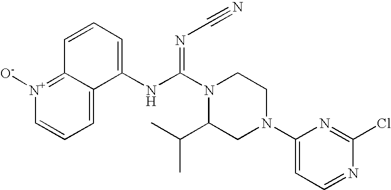 | 1.30 (d) | 449 (M − H)⁻ |
| 8.5 | 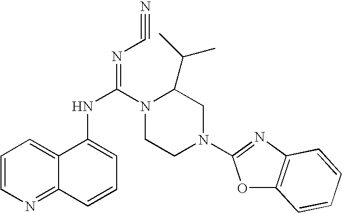 | 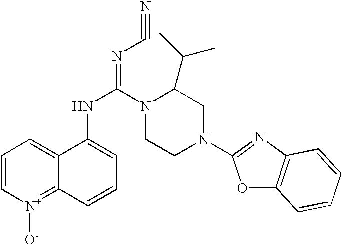 | 1.58 (d) | 456 (M + H)⁺ |
| 8.6 | 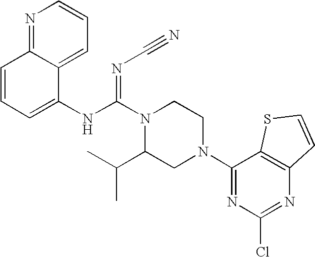 | 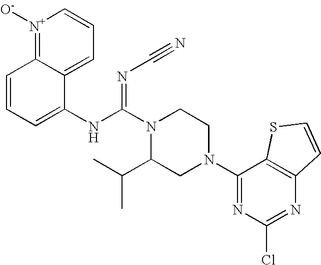 | 1.31 (d) | 505 (M − H)⁻ |
| 8.7 | 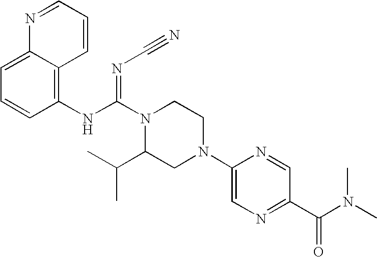 | 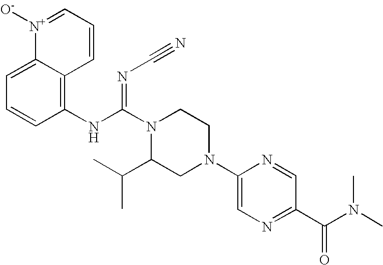 | 1.28 (b) | 488 (M + H)⁺ |
| 8.8 | 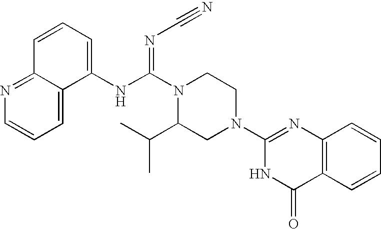 | 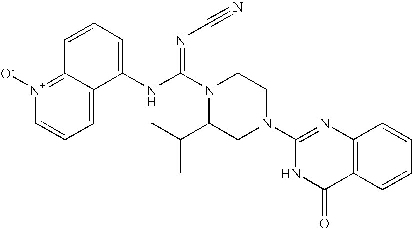 | 1.15 (d) | 483 (M + H)⁺ |
| 8.9 | 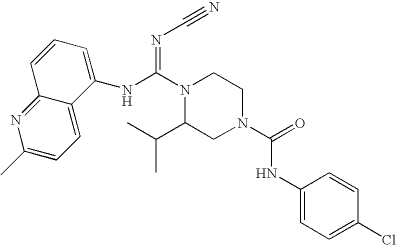 | 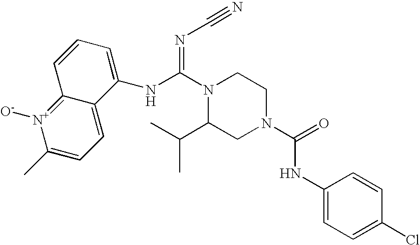 | 2.11 (a) | 506 (M + H)⁺ |

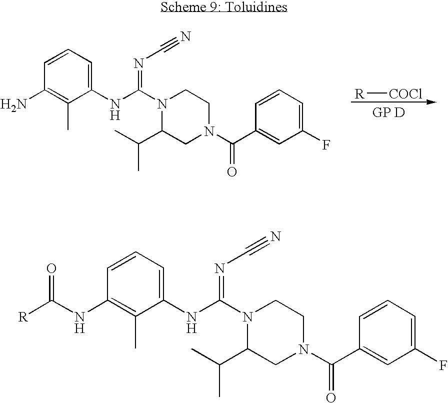

The compounds in table 9 were made according to general procedure D, as illustrated in scheme 9.

TABLE 9
Toluidines

| | | | | Rt/min |
|---|---|---|---|---|
| Ex # | R-COCl | Product | (method) | m/z |
| 9.1 | 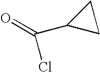 | 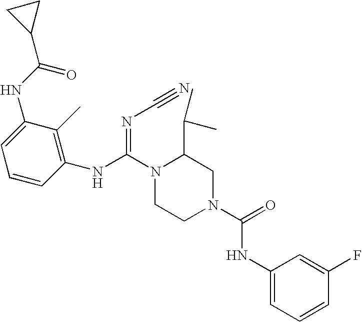 | 1.64 (d) | 506 (M + H)⁺ |
| 9.2 | 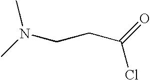 | 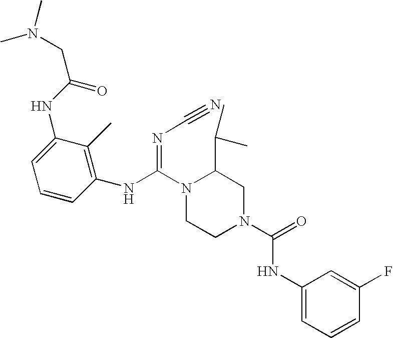 | 1.38 (d) | 523 (M + H)⁺ |
| 9.3 | 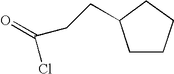 | 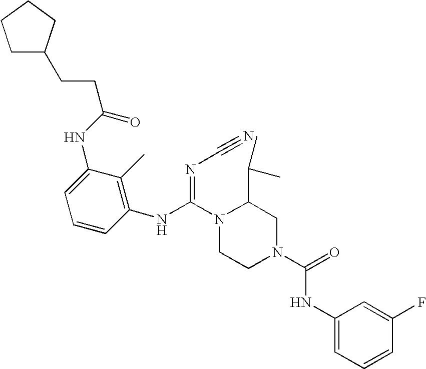 | 2.03 (d) | 562 (M + H)⁺ |
| 9.4 | 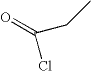 | 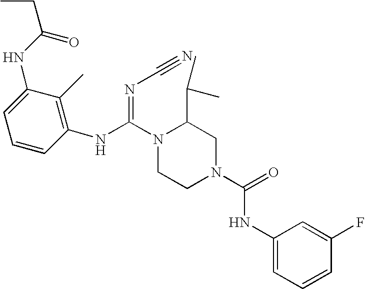 | 1.61 (d) | 494 (M + H)⁺ |
| 9.5 | 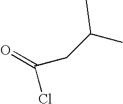 | 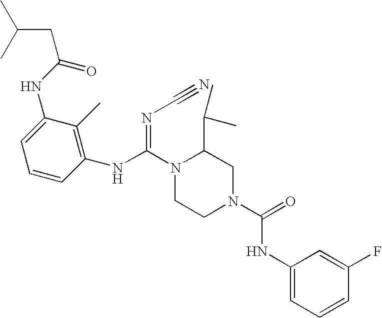 | 1.77 (d) | 522 (M + H)⁺ |
| 9.6 | 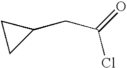 | 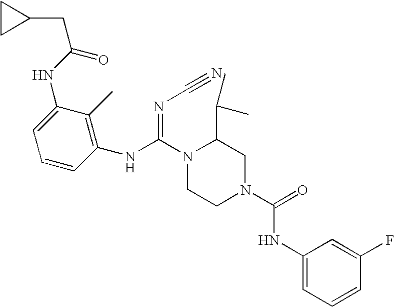 | 1.70 (d) | 520 (M + H)⁺ |
| 9.7 | 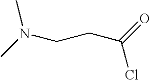 | 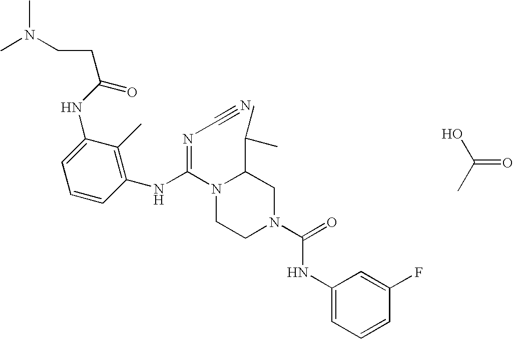 | 1.42 (d) | 537 (M + H)⁺ |
| 9.8 | 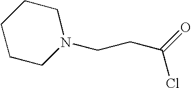 | 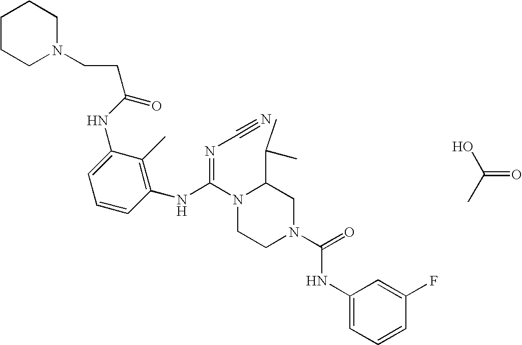 | 1.52 (d) | 577 (M + H)⁺ |
| 9.9 | 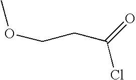 | 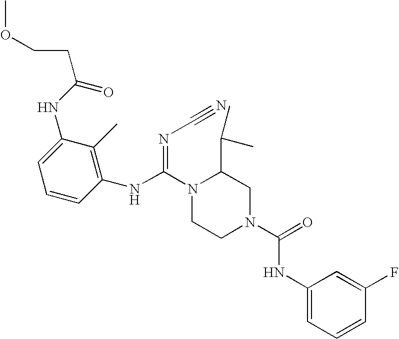 | 1.58 (d) | 524 (M + H)⁺ |
| 9.10 | 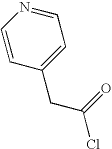 | 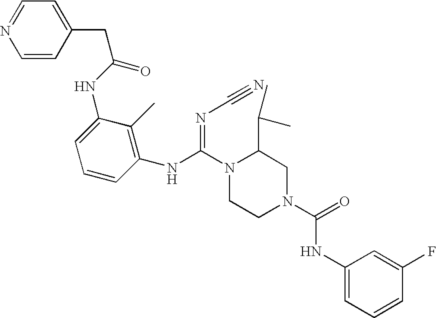 | 1.53 (d) | 557 (M + H)⁺ |

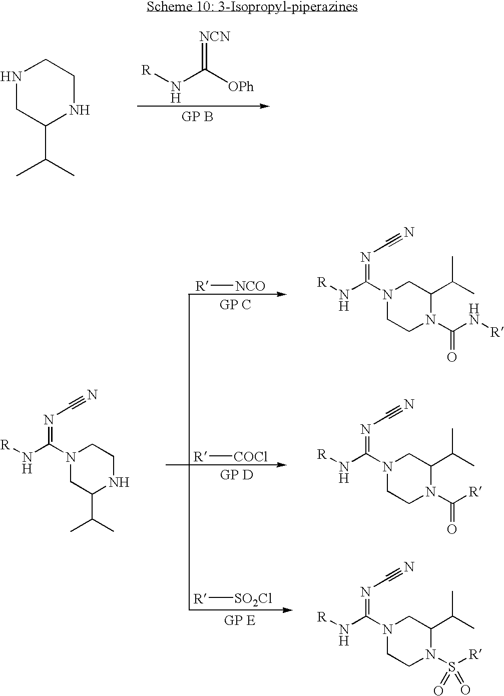

The compounds in table 10 were made according to general procedure B, followed by either general procedure C or D or E as illustrated in scheme 10. The specific procedure used for each compound is listed in table 10.

TABLE 10
3-Isopropyl-piperazines

| | | | | | | Rt/min |
|---|---|---|---|---|---|---|
| Ex # | Reagent1 | Reagent2 | GP | Product | (method) | m/z |
| 10.1 | 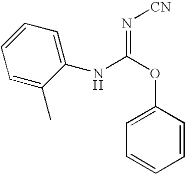 | 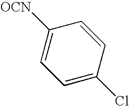 | C | 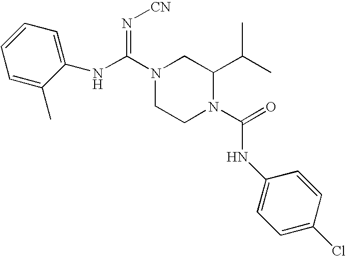 | 2.70 (i) | 439 (M + H)⁺ |
| 10.2 | 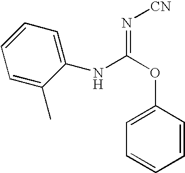 | 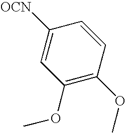 | C | 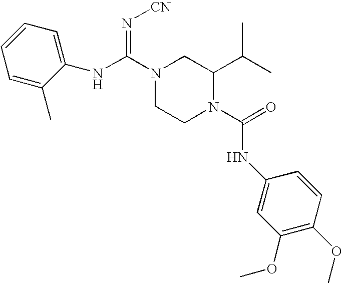 | 2.13 (i) | 465 (M + H)⁺ |
| 10.3 | 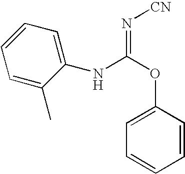 | 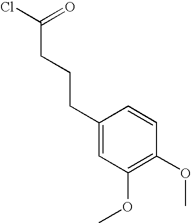 | D | 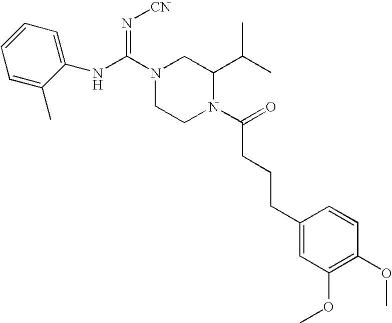 | 2.46 (i) | 492 (M + H)⁺ |
| 10.4 | 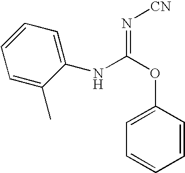 | 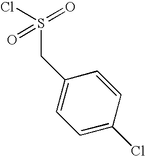 | E | 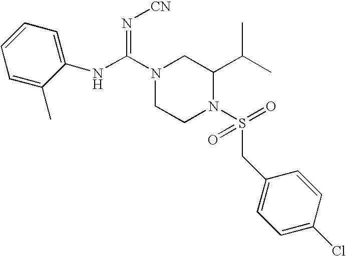 | 2.99 (i) | 474 (M + H)⁺ |
| 10.5 | 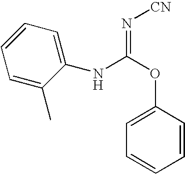 | 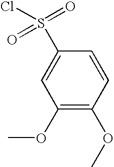 | E | 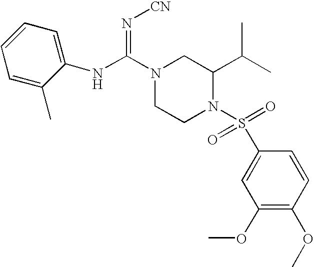 | 2.57 (i) | 486 (M + H)⁺ |
| 10.6 | 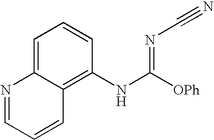 | 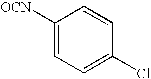 | C | 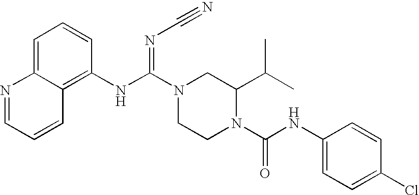 | 3.20 (i) | 474 (M − H)⁻ |

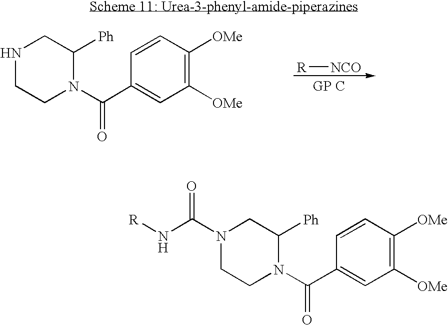

The compounds in table 11 were made according to general procedure C, as illustrated in scheme 11.

TABLE 11
Urea-3-phenyl-amide-piperazines

| | | | Rt/ | | m/z |
|---|---|---|---|---|---|
| Ex # | R-NCO | Product | min | method | (M + H)⁺ |
| 11.1 | 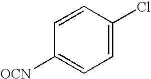 | 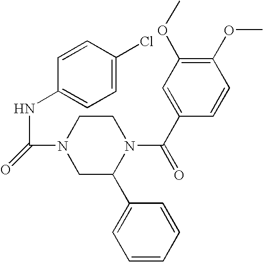 | 2.03 | j | 480.2 |
| 11.2 | 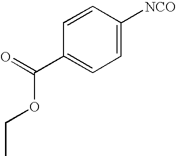 | 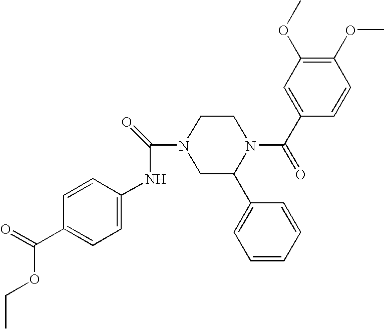 | 1.98 | j | 518.2 |
| 11.3 | 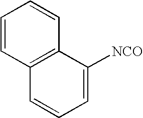 | 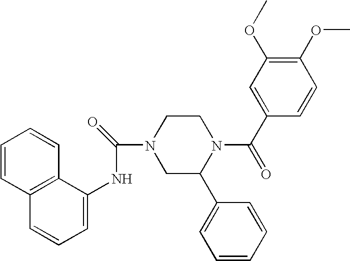 | 1.91 | j | 496.2 |
| 11.4 | 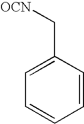 | 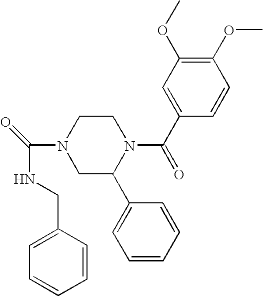 | 1.8 | j | 460.2 |
| 11.5 | 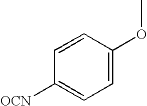 | 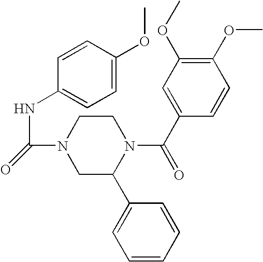 | 1.75 | j | 476.2 |
| 11.6 | 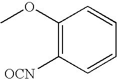 | 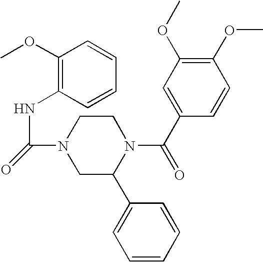 | 1.87 | j | 476.2 |
| 11.7 | 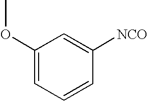 | 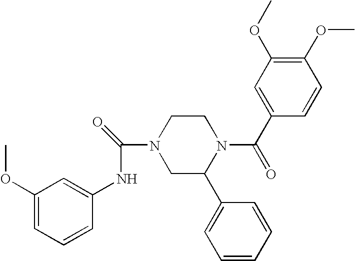 | 1.82 | j | 476.2 |
| 11.8 | 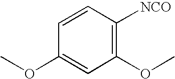 | 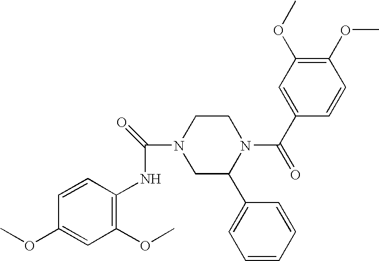 | 1.82 | j | 506.2 |
| 11.9 | 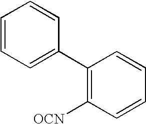 | 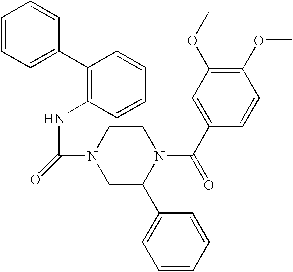 | 2.14 | j | 522.2 |
| 11.10 | 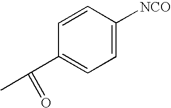 | 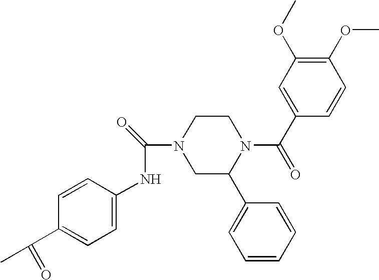 | 1.72 | j | 488.2 |
| 11.11 | 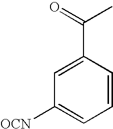 | 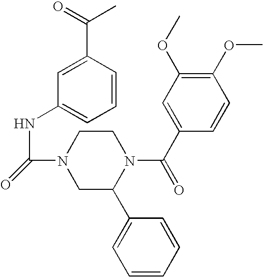 | 1.76 | j | 488.2 |
| 11.12 | 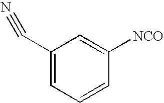 | 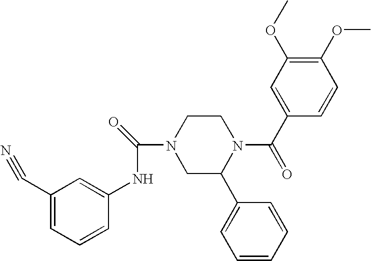 | 1.83 | j | 471.2 |
| 11.13 | 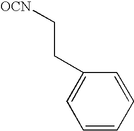 | 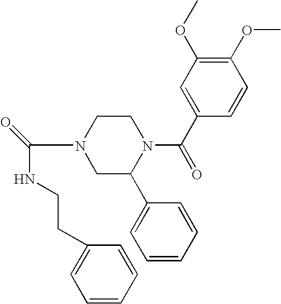 | 1.87 | j | 474.2 |
| 11.14 | 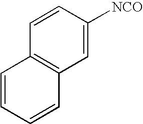 | 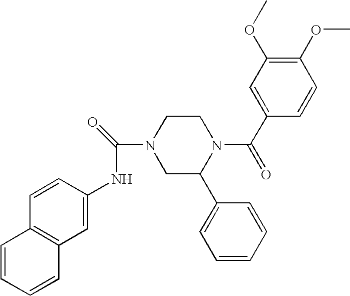 | 2.06 | j | 496.2 |
| 11.15 | 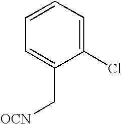 | 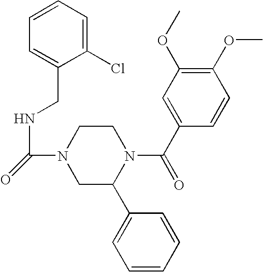 | 1.95 | j | 494.2 |
| 11.16 | 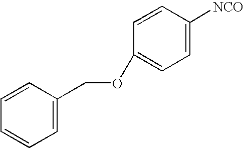 | 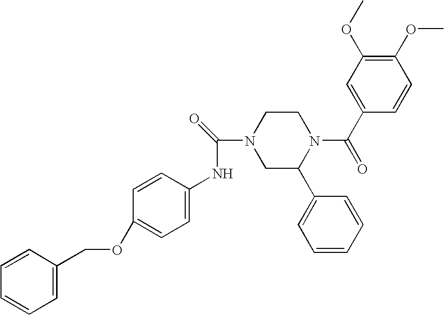 | 2.23 | j | 552.2 |
| 11.17 | 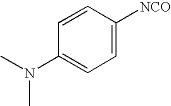 | 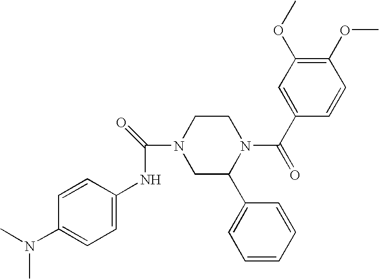 | 1.22 | j | 489.2 |
| 11.18 | 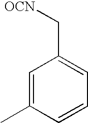 | 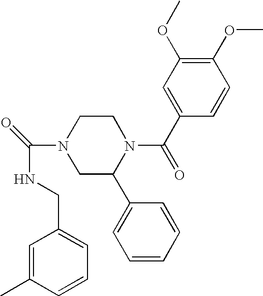 | 1.9 | j | 474.2 |
| 11.19 | 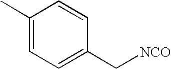 | 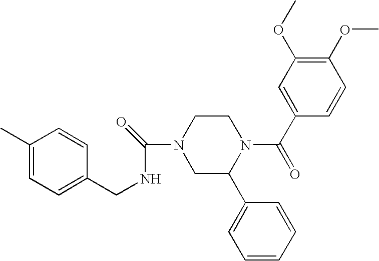 | 1.91 | j | 474.2 |
| 11.20 | 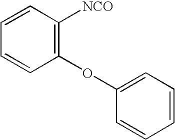 | 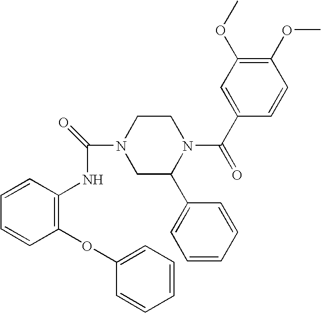 | 2.21 | j | 538.2 |
| 11.21 | 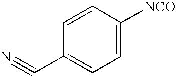 | 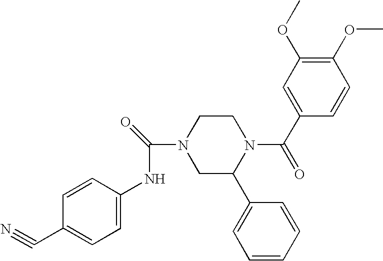 | 1.82 | j | 471.2 |
| 11.22 | 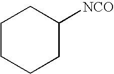 | 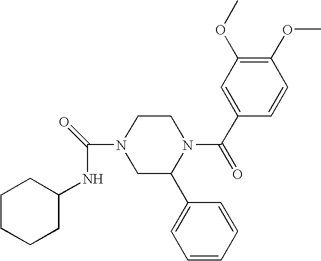 | 1.9 | j | 452.2 |
| 11.23 | 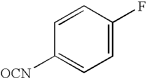 | 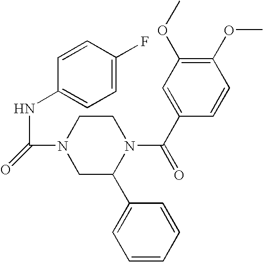 | 1.91 | j | 464.2 |
| 11.24 | 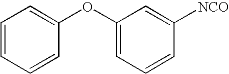 | 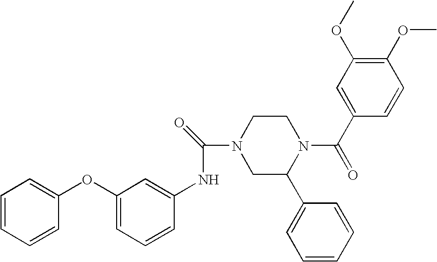 | 2.26 | j | 538.2 |
| 11.25 | 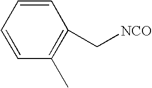 | 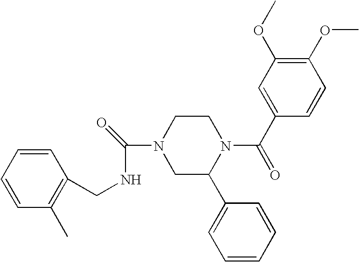 | 1.88 | j | 474.2 |
| 11.26 | 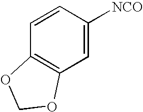 | 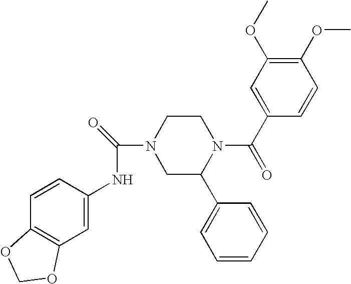 | 1.75 | j | 490.2 |
| 11.27 | 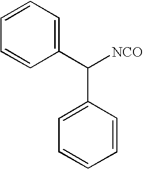 | 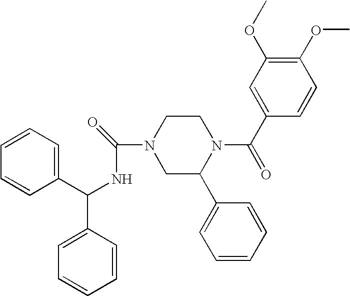 | 2.17 | j | 536.2 |
| 11.28 | 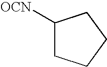 | 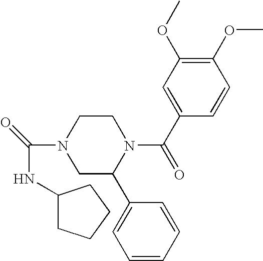 | 1.75 | j | 438.2 |
| 11.29 | 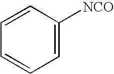 | 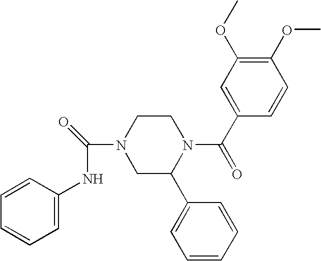 | 1.81 | j | 446.2 |
| 11.30 | 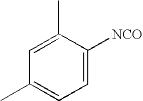 | 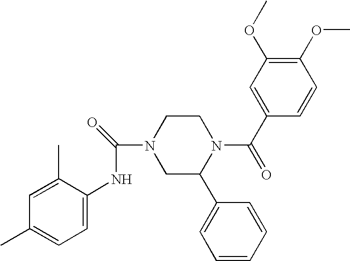 | 1.96 | j | 474.2 |
| 11.31 | 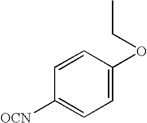 | 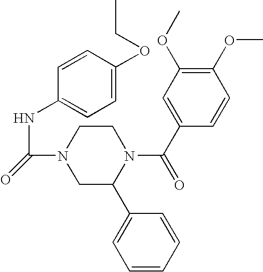 | 1.88 | j | 490.2 |
| 11.32 | 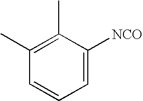 | 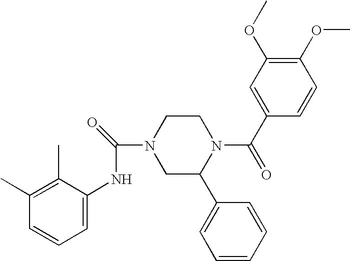 | 1.91 | j | 474.2 |
| 11.33 | 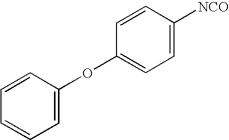 | 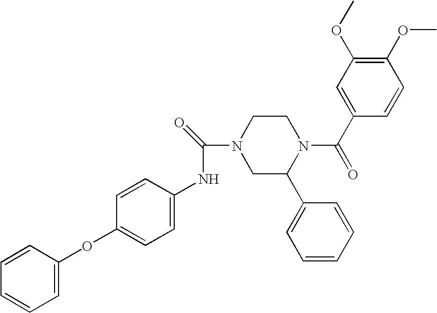 | 2.23 | j | 538.2 |
| 11.34 | 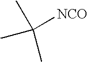 | 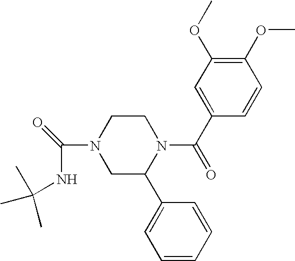 | 1.8 | j | 426.2 |
| 11.35 | 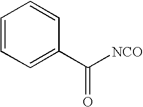 | 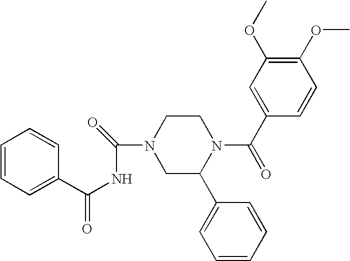 | 1.67 | j | 475.2 |
| 11.36 | 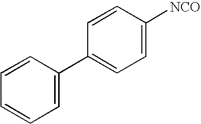 | 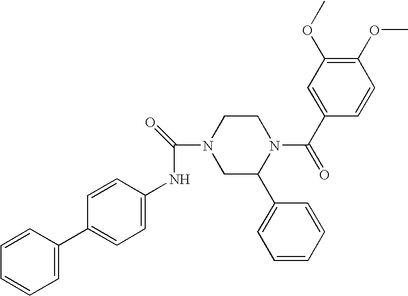 | 2.23 | j | 522.2 |
| 11.37 | 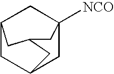 | 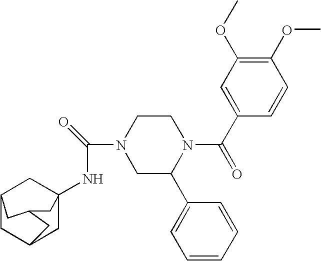 | 2.26 | j | 504.2 |
| 11.38 | 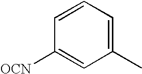 | 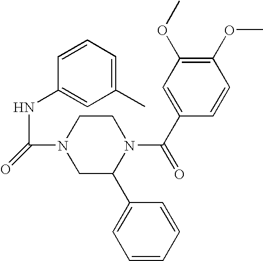 | 1.95 | j | 460.2 |

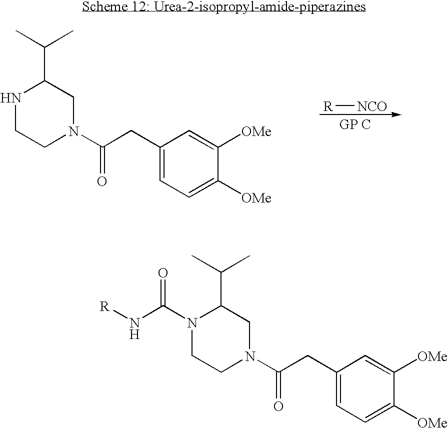

The compounds in table 12 were made according to general procedure C, as illustrated in scheme 12.

TABLE 12
Urea-2-isopropyl-amide-piperazines

| | | | | | m/z |
|---|---|---|---|---|---|
| Ex # | R-NCO | Product | Rt/min | method | (M + H)⁺ |
| 12.1 | 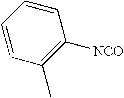 | 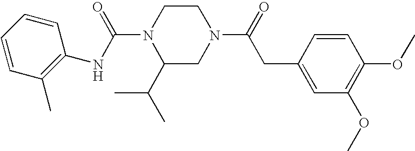 | 1.71 | j | 440.2 |
| 12.2 | 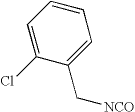 | 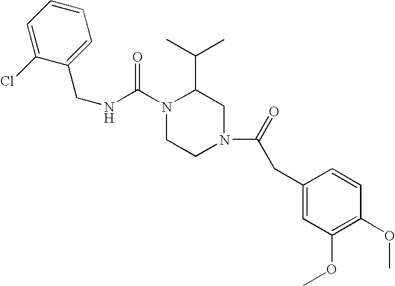 | 2.45 | j | 474 |
| 12.3 | 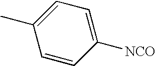 | 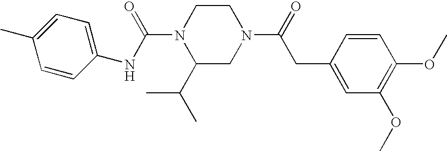 | 1.86 | j | 440.2 |
| 12.4 | 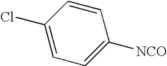 | 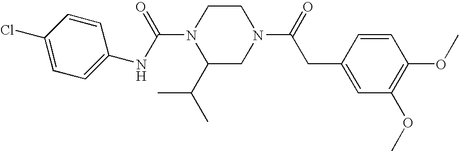 | 1.95 | j | 460.2 |
| 12.5 | 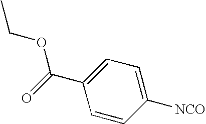 | 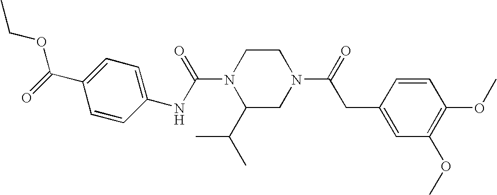 | 1.92 | j | 498.3 |
| 12.6 | 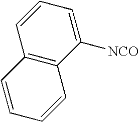 | 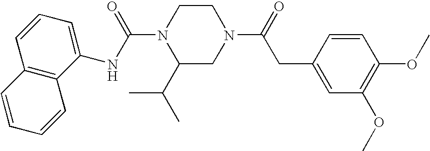 | 1.86 | j | 476.2 |
| 12.7 | 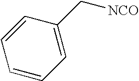 | 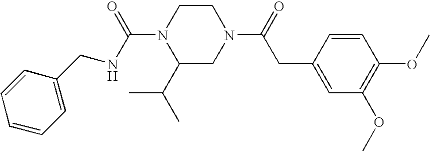 | 1.69 | j | 440.2 |
| 12.8 | 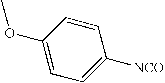 | 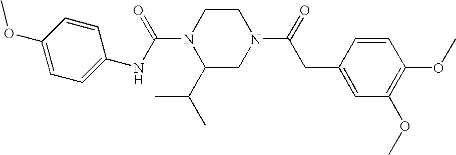 | 1.66 | j | 456.2 |
| 12.9 | 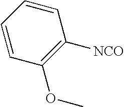 | 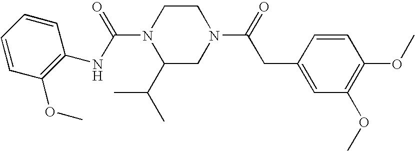 | 1.77 | j | 456.2 |
| 12.10 | 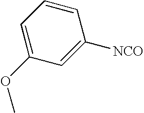 | 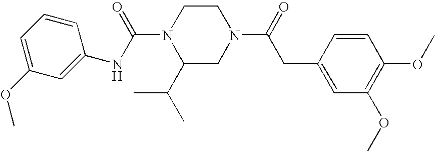 | 1.73 | j | 456.2 |
| 12.11 | 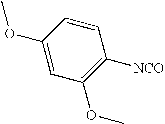 | 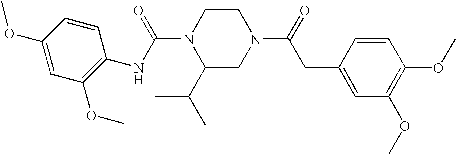 | 1.71 | j | 486.3 |
| 12.12 | 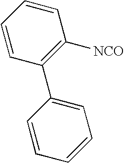 | 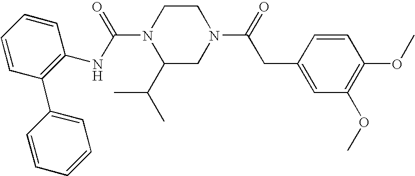 | 2.05 | j | 503.2 |
| 12.13 | 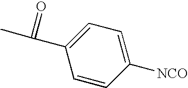 | 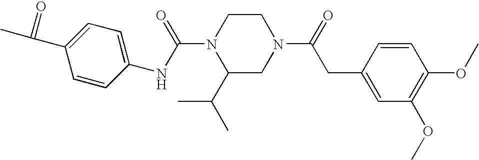 | 1.63 | j | 468.2 |
| 12.14 | 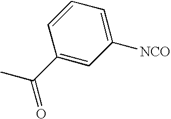 | 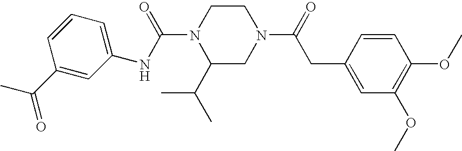 | 1.66 | j | 468.2 |
| 12.15 | 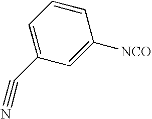 | 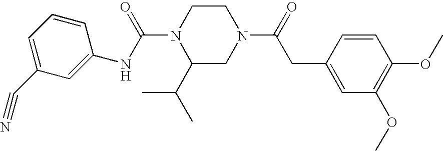 | 1.75 | j | 451.2 |
| 12.16 | 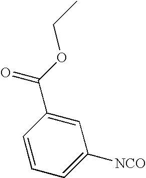 | 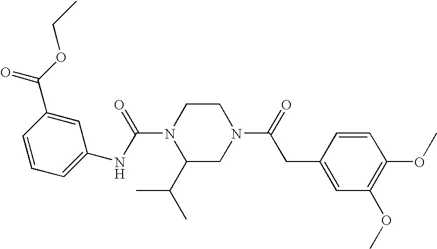 | 1.91 | j | 498.3 |
| 12.17 | 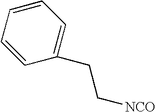 | 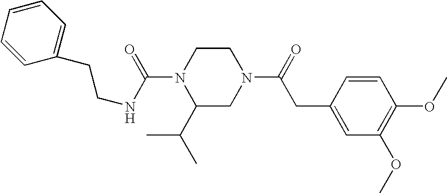 | 1.77 | j | 454.3 |
| 12.18 | 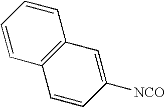 | 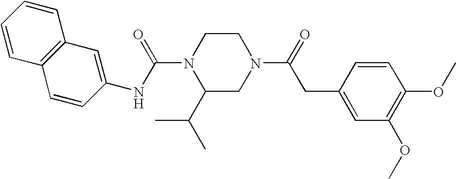 | 2.01 | j | 476.2 |
| 12.19 | 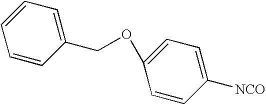 | 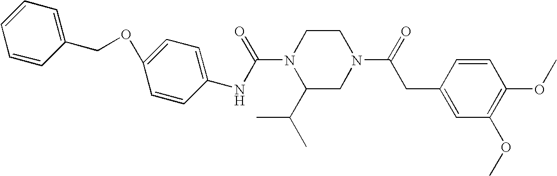 | 2.12 | j | 532.3 |
| 12.20 | 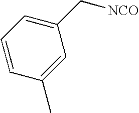 | 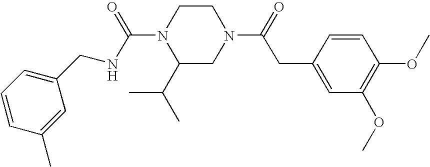 | 1.83 | j | 454.3 |
| 12.21 | 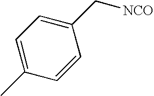 | 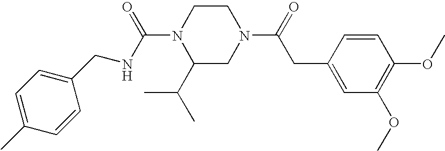 | 1.83 | j | 454.3 |
| 12.22 | 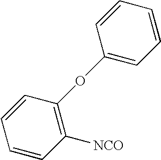 | 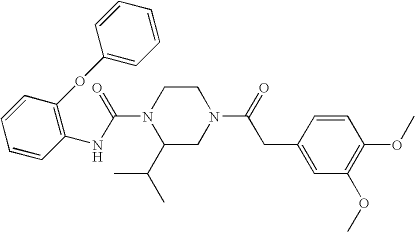 | 2.12 | j | 518.3 |
| 12.23 | 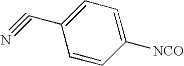 | 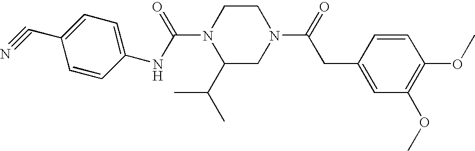 | 1.72 | j | 451.2 |
| 12.24 | 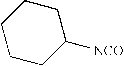 | 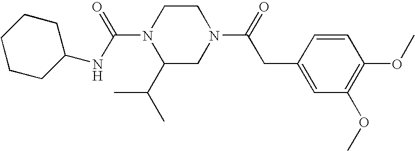 | 1.77 | j | 432.3 |
| 12.25 | 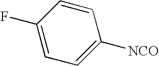 | 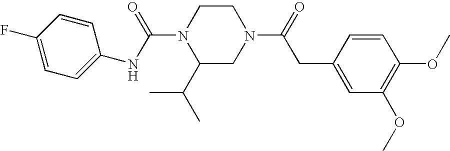 | 1.77 | j | 444.2 |
| 12.26 | 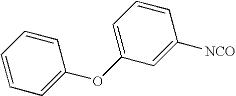 | 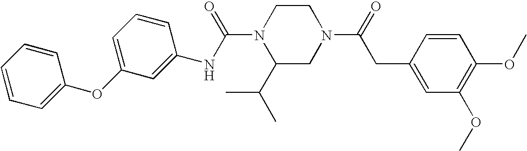 | 2.2 | j | 518.3 |
| 12.27 | 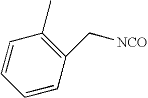 | 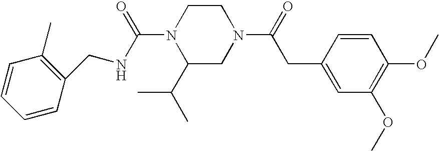 | 1.81 | j | 454.3 |
| 12.28 | 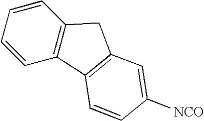 | 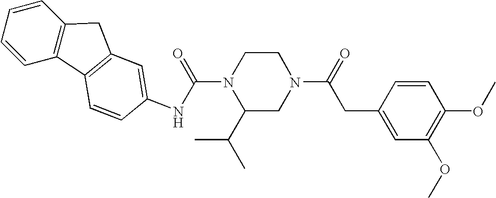 | 2.24 | j | 514.2 |
| 12.29 | 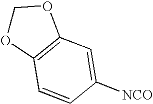 | 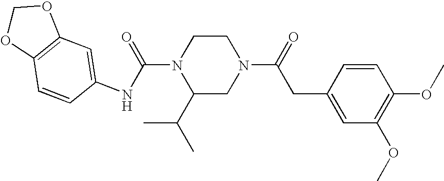 | 1.75 | j | 470.2 |
| 12.30 | 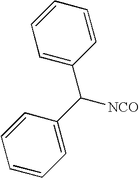 | 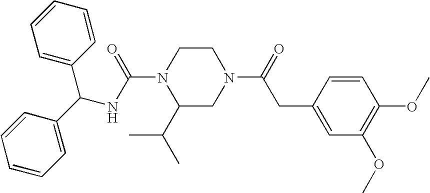 | 2.17 | j | 516.2 |
| 12.31 | 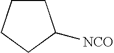 | 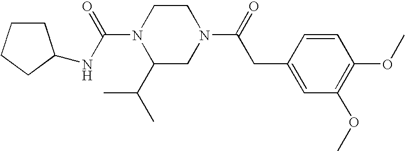 | 1.75 | j | 418.2 |
| 12.32 | 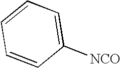 | 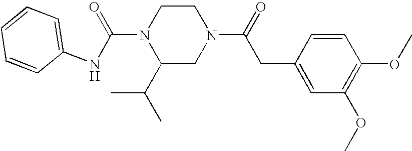 | 1.81 | j | 426.2 |
| 12.33 | 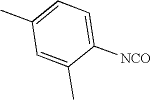 | 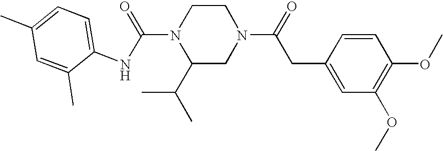 | 1.96 | j | 454.2 |
| 12.34 | 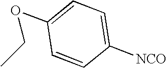 | 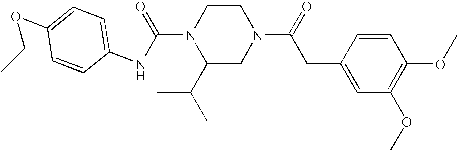 | 1.88 | j | 470.2 |
| 12.35 | 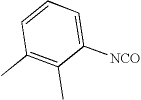 | 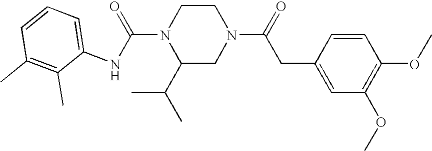 | 1.91 | j | 454.2 |
| 12.36 | 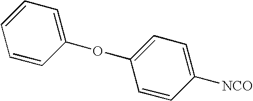 | 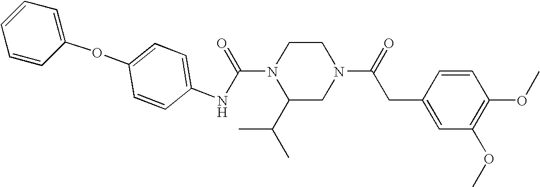 | 2.23 | j | 518.2 |
| 12.37 | 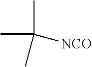 | 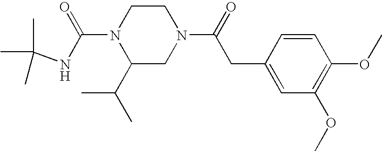 | 1.8 | j | 406.2 |
| 12.38 | 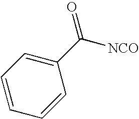 | 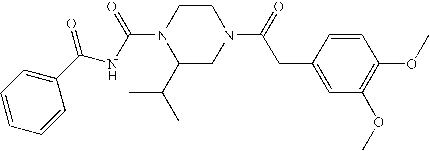 | 1.67 | j | 454.2 |
| 12.39 | 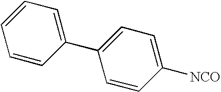 | 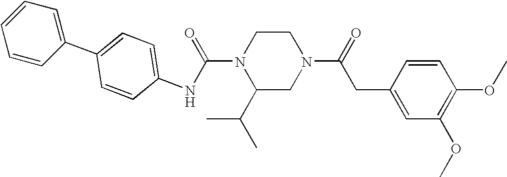 | 2.23 | j | 502.2 |
| 12.40 | 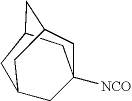 | 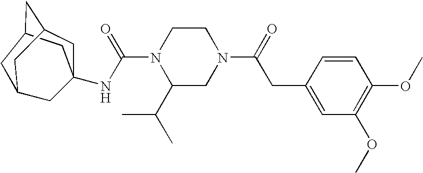 | 2.26 | j | 484.2 |
| 12.41 | 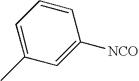 | 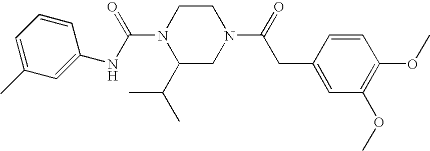 | 1.95 | j | 440.2 |

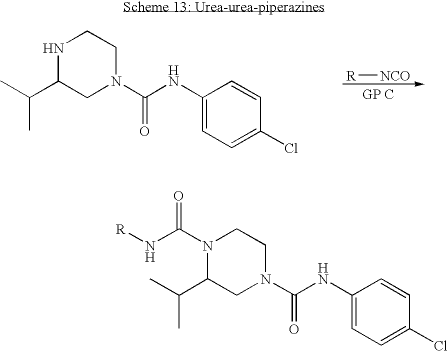

The compounds in table 13 were made according to general procedure C, as illustrated in scheme 13.

TABLE 13
Urea-urea-piperazines

| | | | RT/min | |
|---|---|---|---|---|
| Ex # | R—NCO | MOL STRUCTURE | (method) | m/z |
| 13.1 | 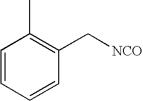 | 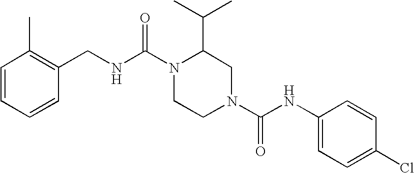 | 3.07 (i) | 429 (M + H)⁺ |
| 13.2 | 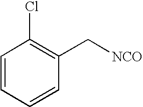 | 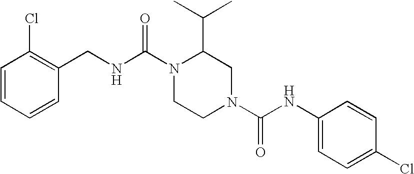 | 3.12 (i) | 449 (M + H)⁺ |
| 13.3 | 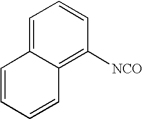 | 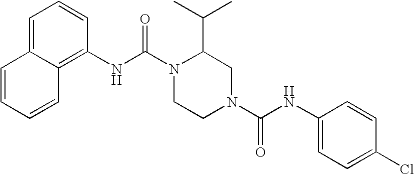 | 3.17 (i) | 451 (M + H)⁺ |
| 13.4 | 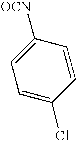 | 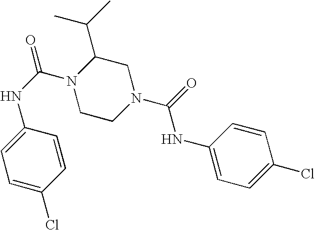 | 3.30 (i) | 434 (M − H)⁻ |

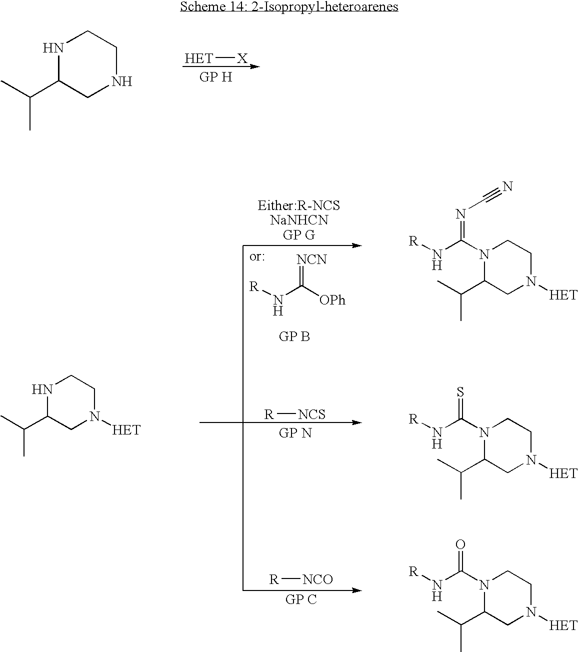

The compounds in table 14 were made according to general procedure H, followed by either general procedure B or G or C or N as illustrated in scheme 14. The specific procedure used for each compound is listed in table 14.

TABLE 14
2-Isopropyl-heteroarenes

| Ex # | Het-X | Reagent | GP |
|---|---|---|---|
| 14.1 | 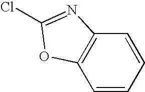 | 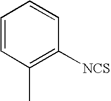 | G |
| 14.2 | 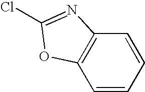 | 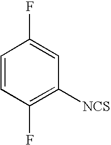 | G |
| 14.3 | 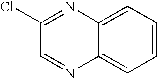 | 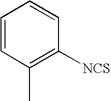 | G |
| 14.4 | 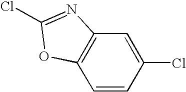 | 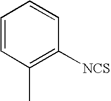 | G |
| 14.5 | 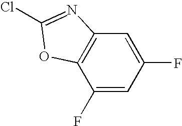 | 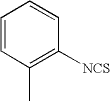 | G |
| 14.6 | 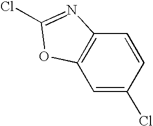 | 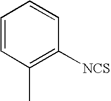 | G |
| 14.7 | 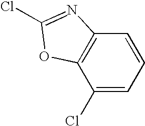 | 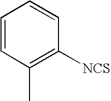 | G |
| 14.8 | 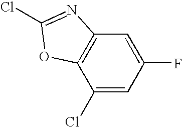 | 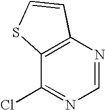 | G |
| 14.9 | 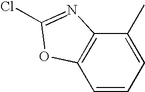 | 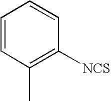 | G |
| 14.10 | 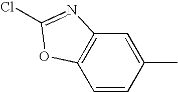 | 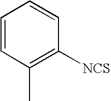 | G |
| 14.11 | 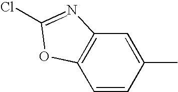 | 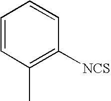 | G |
| 14.12 | 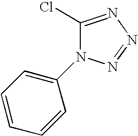 | 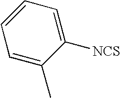 | G |
| 14.13 | 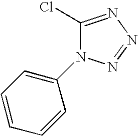 | 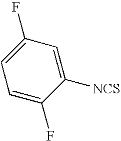 | G |
| 14.14 | 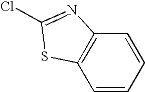 | 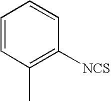 | G |
| 14.15 | 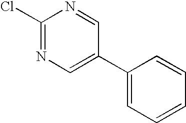 | 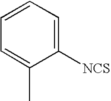 | G |
| 14.16 | 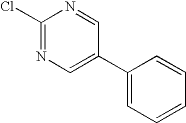 | 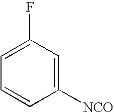 | C |
| 14.17 | 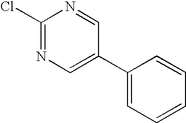 | 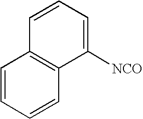 | C |
| 14.18 | 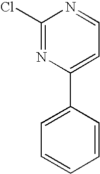 | 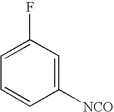 | C |
| 14.19 | 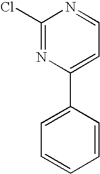 | 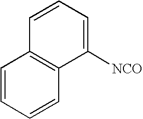 | C |
| 14.20 | 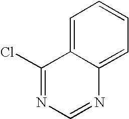 | 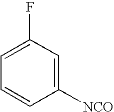 | C |
| 14.21 | 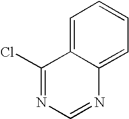 | 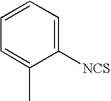 | G |
| 14.22 | 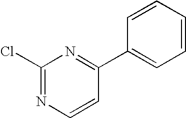 | 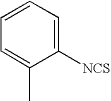 | G |
| 14.23 | 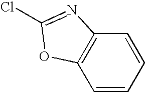 | 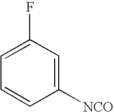 | C |
| 14.24 | 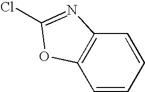 | 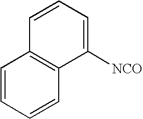 | C |
| 14.25 | 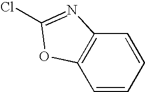 | 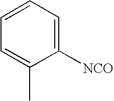 | C |
| 14.26 | 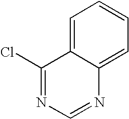 | 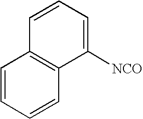 | C |
| 14.27 | 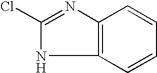 | 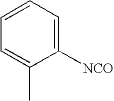 | C |
| 14.28 | 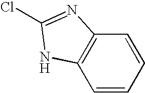 | 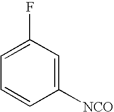 | C |
| 14.29 | 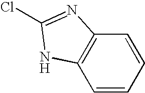 | 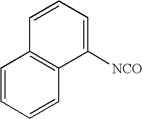 | C |
| 14.30 | 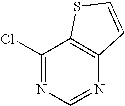 | 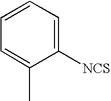 | G |
| 14.31 | 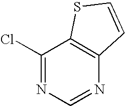 | 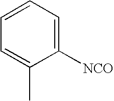 | C |
| 14.32 | 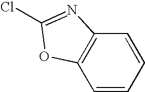 | 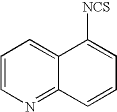 | G |
| 14.33 | 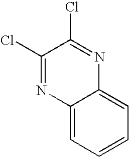 | 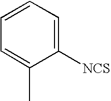 | G |
| 14.34 | 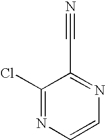 | 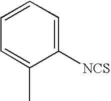 | G |
| 14.35 | 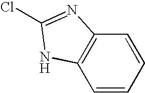 | 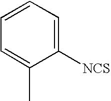 | G |
| 14.36 | 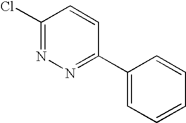 | 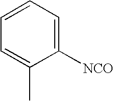 | C |
| 14.37 | 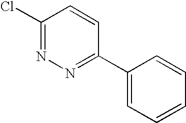 | 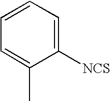 | G |
| 14.38 | 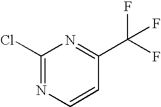 | 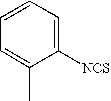 | G |
| 14.39 | 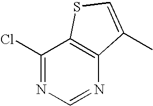 | 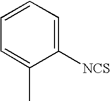 | G |
| 14.40 | 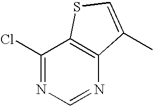 | 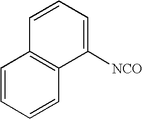 | C |
| 14.41 | 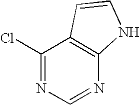 | 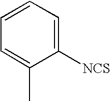 | G |
| 14.42 | 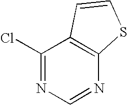 | 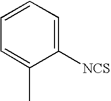 | G |
| 14.43 | 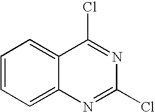 | 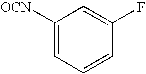 | C |
| 14.44 | 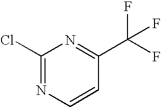 | 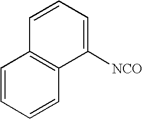 | C |
| 14.45 | 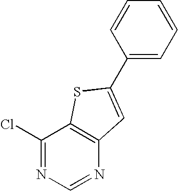 | 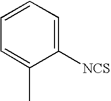 | G |
| 14.46 | 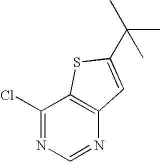 | 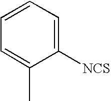 | G |
| 14.47 | 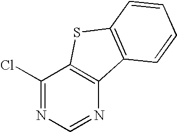 | 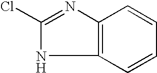 | G |
| 14.48 | 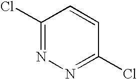 | 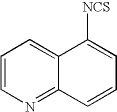 | G |
| 14.49 | 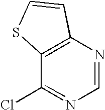 | 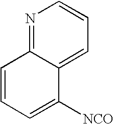 | C |
| 14.50 | 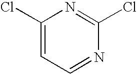 | 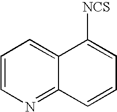 | N |
| 14.51 | 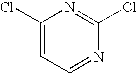 | 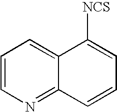 | N |
| 14.52 | 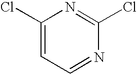 | 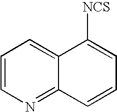 | G |
| 14.53 | 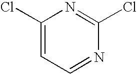 | 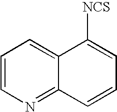 | G |
| 14.54 | 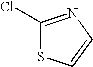 | 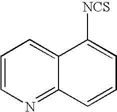 | G |
| 14.55 | 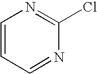 | 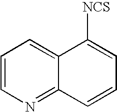 | G |
| 14.56 | 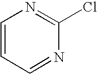 | 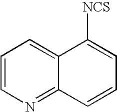 | G |
| 14.57 | 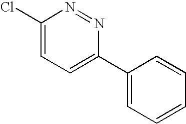 | 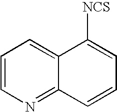 | G |
| 14.58 | 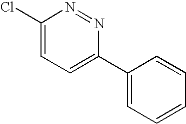 | 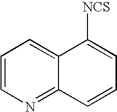 | G |
| 14.59 | 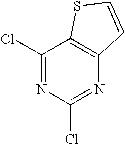 | 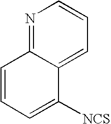 | G |
| 14.60 | 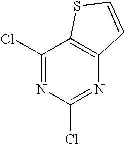 | 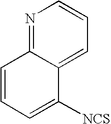 | N |
| 14.61 | 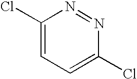 | 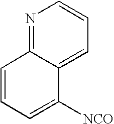 | C |
| 14.62 | 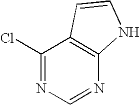 | 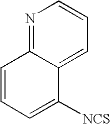 | B |
| 14.63 | 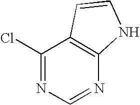 | 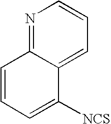 | N |
| 14.64 | 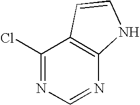 | 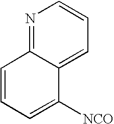 | C |
| 14.65 | 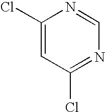 | 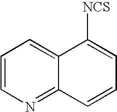 | G |
| 14.66 | 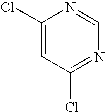 | 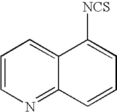 | G |
| 14.67 | 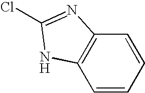 | 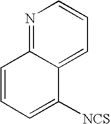 | N |
| 14.68 | 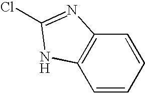 | 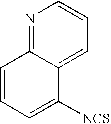 | G |
| 14.69 | 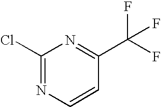 | 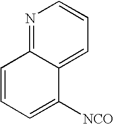 | C |
| 14.70 | 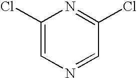 | 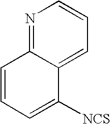 | G |
| 14.71 | 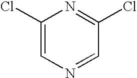 | 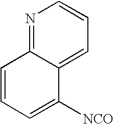 | C |
| 14.72 | 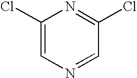 | 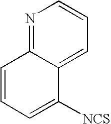 | N |
| 14.73 | 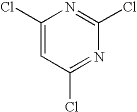 | 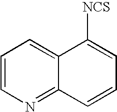 | N |
| 14.74 | 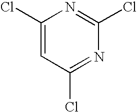 | 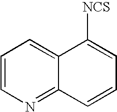 | N |
| 14.75 | 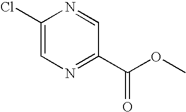 | 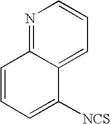 | G |
| 14.76 | 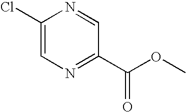 | 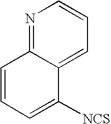 | N |
| 14.77 | 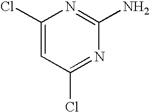 | 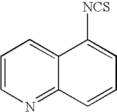 | G |
| 14.78 | 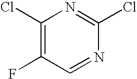 | 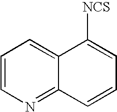 | G |
| 14.79 | 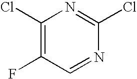 | 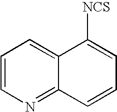 | G |
| 14.80 | 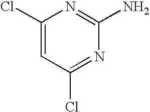 | 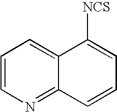 | G |
| 14.81 | 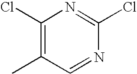 | 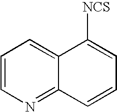 | G |
| 14.82 | 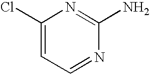 | 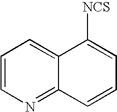 | G |
| 14.83 | 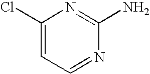 | 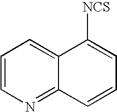 | G |
| 14.84 | 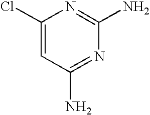 | 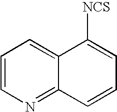 | G |
| 14.45 | 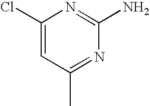 | 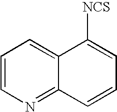 | G |
| 14.86 | 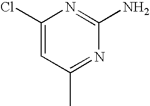 | 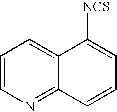 | G |
| 14.87 | 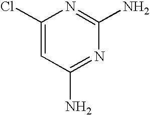 | 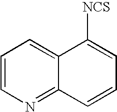 | G |
| 14.88 | 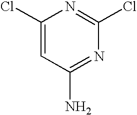 | 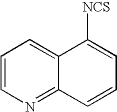 | G |
| 14.89 | 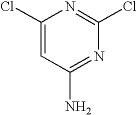 | 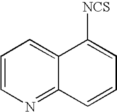 | G |
| 14.90 | 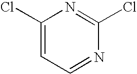 | 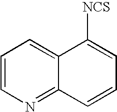 | C |
| 14.91 | 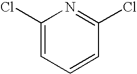 | 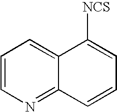 | G |
| 14.92 | 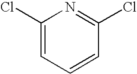 | 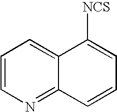 | G |
| 14.93 | 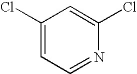 | 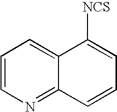 | G |
| 14.94 | 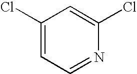 | 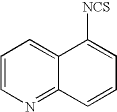 | G |
| 14.95 | 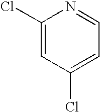 | 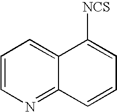 | G |
| 14.96 | 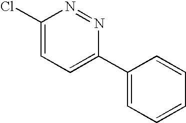 | 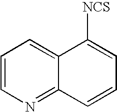 | C |
| 14.97 | 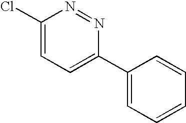 | 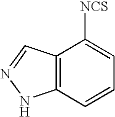 | G |
| 14.98 | 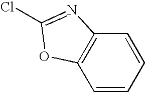 | 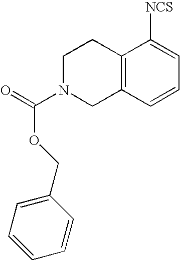 | G |
| 14.99 | 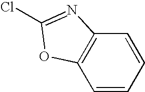 | 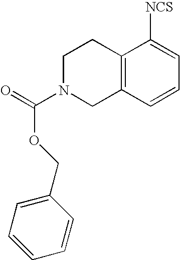 | G |
| 14.100 | 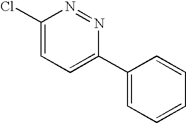 | 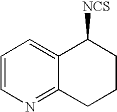 | G |
| 14.101 | 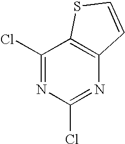 | 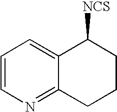 | N |
| 14.102 | 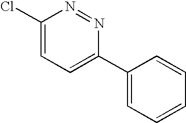 | 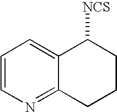 | N |
| 14.103 | 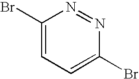 | 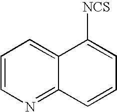 | G |
| 14.104 | 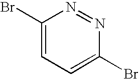 | 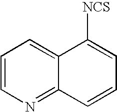 | G |
| 14.105 | 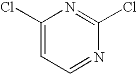 | 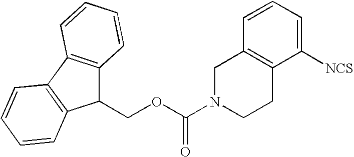 | G |
| 14.106 | 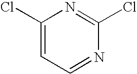 | 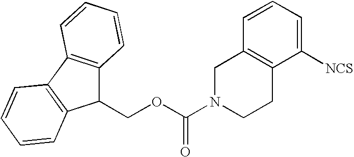 | G |
| 14.107 | 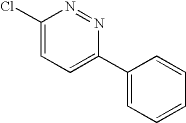 | 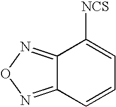 | N |
| 14.108 | 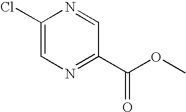 | 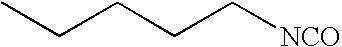 | C |
| 14.109 | 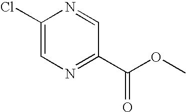 | 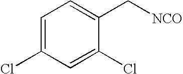 | C |
| 14.110 | 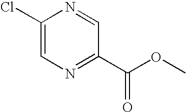 | 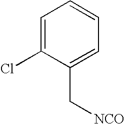 | C |
| 14.111 | 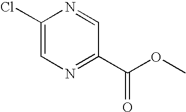 | 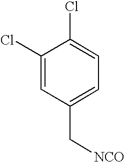 | C |
| 14.112 | 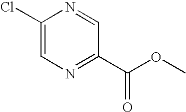 | 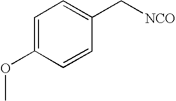 | C |
| 14.113 | 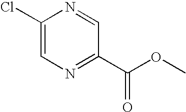 | 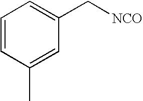 | C |
| 14.114 | 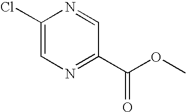 | 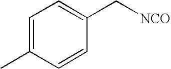 | C |
| 14.115 | 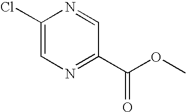 | 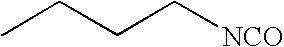 | C |
| 14.116 | 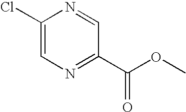 | 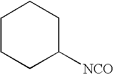 | C |
| 14.117 | 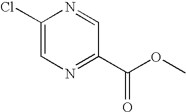 | 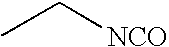 | C |
| 14.118 | 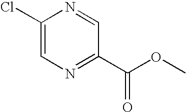 | 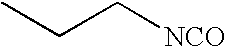 | C |
| 14.119 | 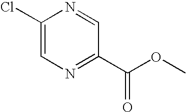 | 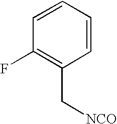 | C |
| 14.120 | 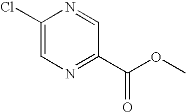 | 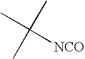 | C |
| 14.121 | 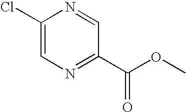 | 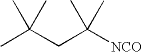 | C |
| 14.122 | 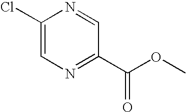 | 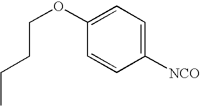 | C |
| 14.123 | 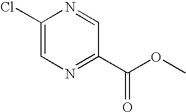 | 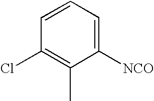 | C |
| 14.124 | 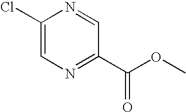 | 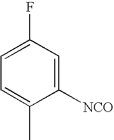 | C |
| 14.125 | 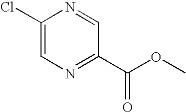 | 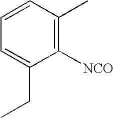 | C |
| 14.126 | 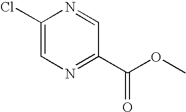 | 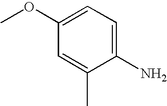 | C |
| 14.127 | 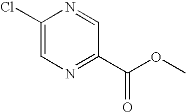 | 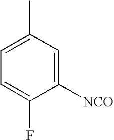 | C |
| 14.128 | 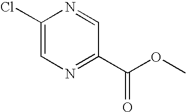 | 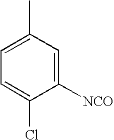 | C |
| 14.129 | 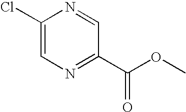 | 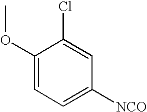 | C |
| 14.130 | 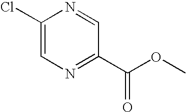 | 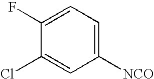 | C |
| 14.131 | 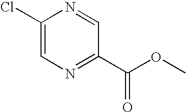 | 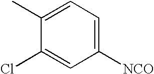 | C |
| 14.132 | 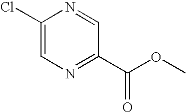 | 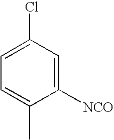 | C |
| 14.133 | 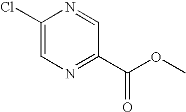 | 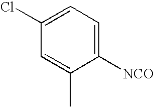 | C |
| 14.134 | 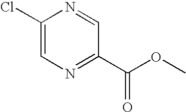 | 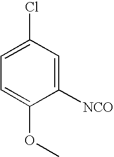 | C |
| 14.135 | 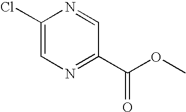 | 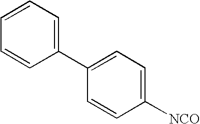 | C |
| 14.136 | 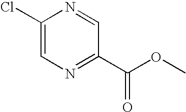 | 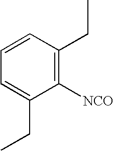 | C |
| 14.137 | 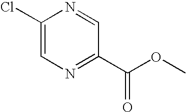 | 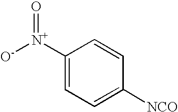 | C |
| 14.138 | 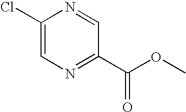 | 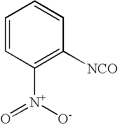 | C |
| 14.139 | 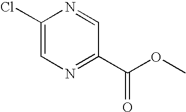 | 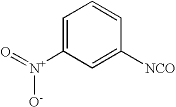 | C |
| 14.140 | 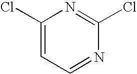 | 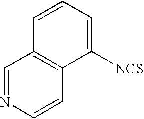 | G |
| 14.141 | 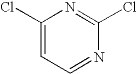 | 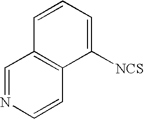 | G |
| 14.142 | 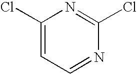 | 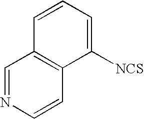 | G |
| 14.143 | 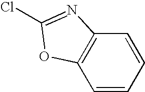 | 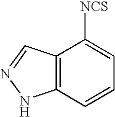 | G |
| 14.144 | 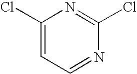 | 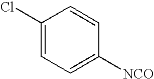 | C |
| 14.145 | 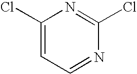 | 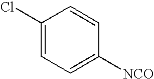 | C |
| 14.146 | 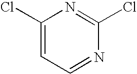 | 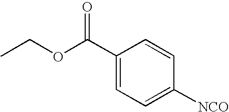 | C |
| 14.147 | 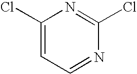 | 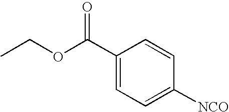 | C |
| 14.148 | 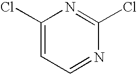 | 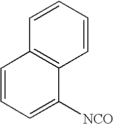 | C |
| 14.149 | 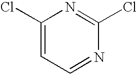 | 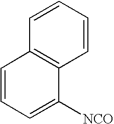 | C |
| 14.150 | 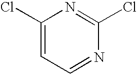 | 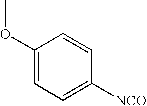 | C |
| 14.151 | 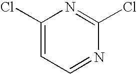 | 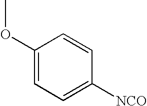 | C |
| 14.152 | 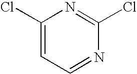 | 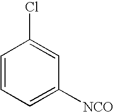 | C |
| 14.153 | 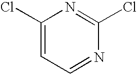 | 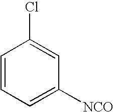 | C |
| 14.154 | 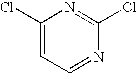 | 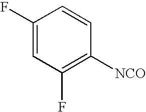 | C |
| 14.155 | 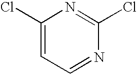 | 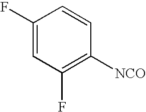 | C |
| 14.156 | 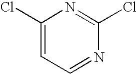 | 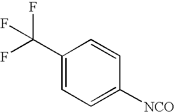 | C |
| 14.157 | 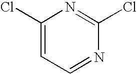 | 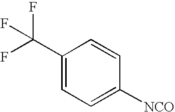 | C |
| 14.158 | 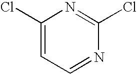 | 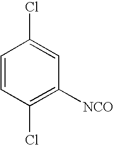 | C |
| 14.159 | 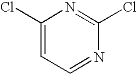 | 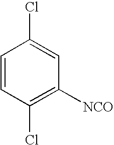 | C |
| 14.160 | 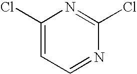 | 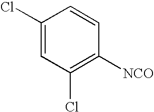 | C |
| 14.161 | 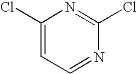 | 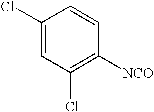 | C |
| 14.162 | 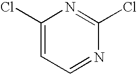 | 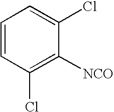 | C |
| 14.163 | 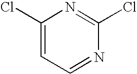 | 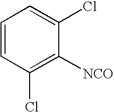 | C |
| 14.164 | 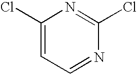 | 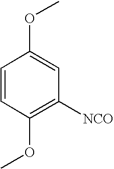 | C |
| 14.165 | 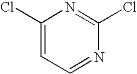 | 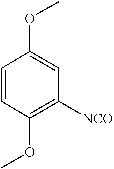 | C |
| 14.166 | 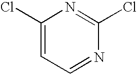 | 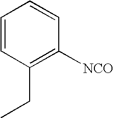 | C |
| 14.167 | 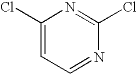 | 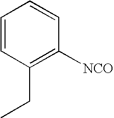 | C |
| 14.168 | 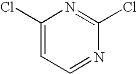 | 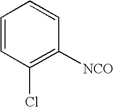 | C |
| 14.169 | 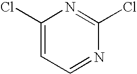 | 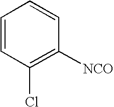 | C |
| 14.170 | 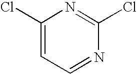 | 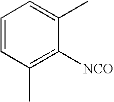 | C |
| 14.171 | 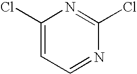 | 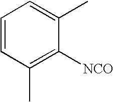 | C |
| 14.172 | 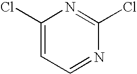 | 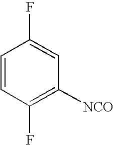 | C |
| 14.173 | 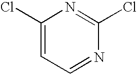 | 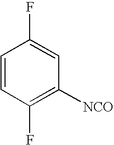 | C |
| 14.174 | 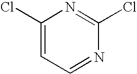 | 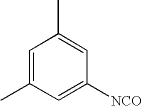 | C |
| 14.175 | 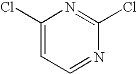 | 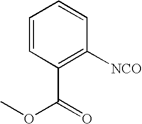 | C |
| 14.176 | 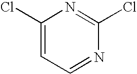 | 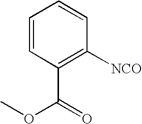 | C |
| 14.177 | 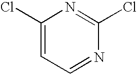 | 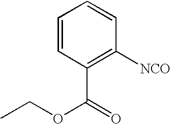 | C |
| 14.178 | 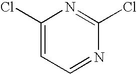 | 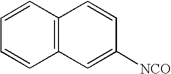 | C |
| 14.179 | 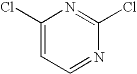 | 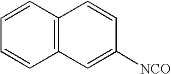 | C |
| 14.180 | 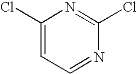 | 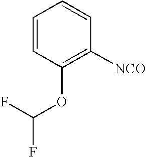 | C |
| 14.181 | 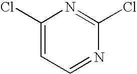 | 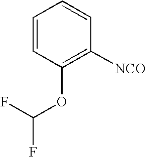 | C |
| 14.182 | 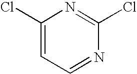 | 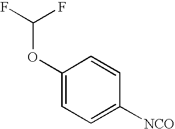 | C |
| 14.183 | 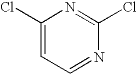 | 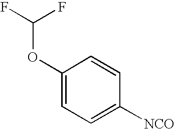 | C |
| 14.184 | 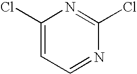 | 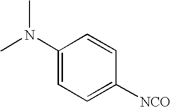 | C |
| 14.185 | 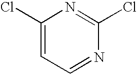 | 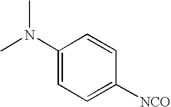 | C |
| 14.186 | 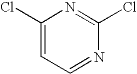 | 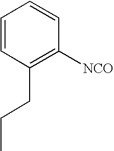 | C |
| 14.187 | 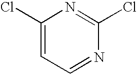 | 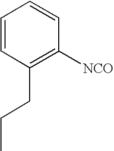 | C |
| 14.188 | 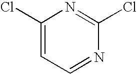 | 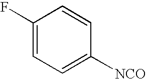 | C |
| 14.189 | 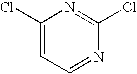 | 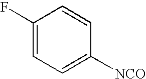 | C |
| 14.190 | 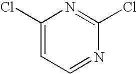 | 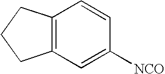 | C |
| 14.191 | 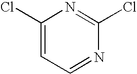 | 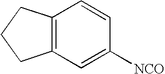 | C |
| 14.192 | 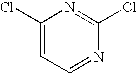 | 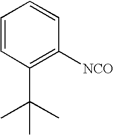 | C |
| 14.193 | 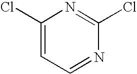 | 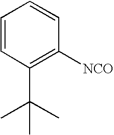 | C |
| 14.194 | 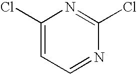 | 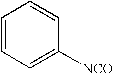 | C |
| 14.195 | 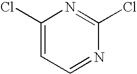 | 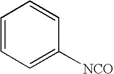 | C |
| 14.196 | 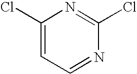 | 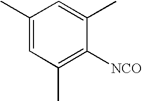 | C |
| 14.197 | 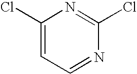 | 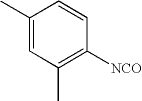 | C |
| 14.198 | 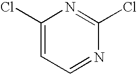 | 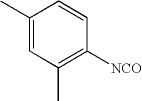 | C |
| 14.199 | 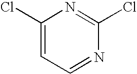 | 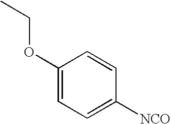 | C |
| 14.200 | 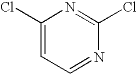 | 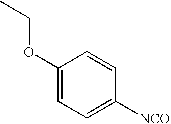 | C |
| 14.201 | 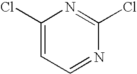 | 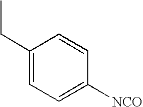 | C |
| 14.202 | 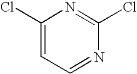 | 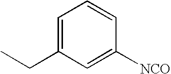 | C |
| 14.203 | 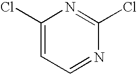 | 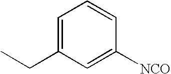 | C |
| 14.204 | 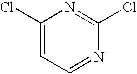 | 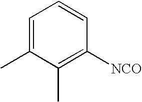 | C |
| 14.205 | 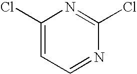 | 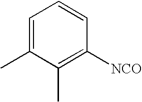 | C |
| 14.206 | 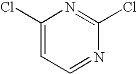 | 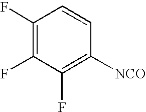 | C |
| 14.207 | 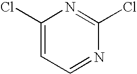 | 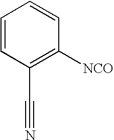 | C |
| 14.208 | 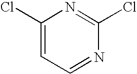 | 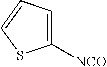 | C |
| 14.209 | 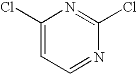 | 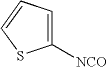 | C |
| 14.210 | 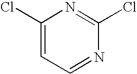 | 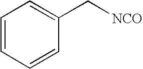 | C |
| 14.211 | 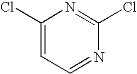 | 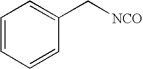 | C |
| 14.212 | 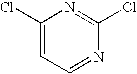 | 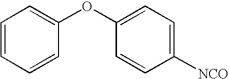 | C |
| 14.213 | 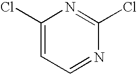 | 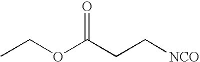 | C |
| 14.214 | 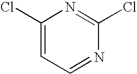 | 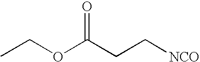 | C |
| 14.215 | 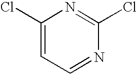 | 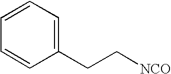 | C |
| 14.216 | 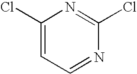 | 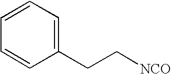 | C |
| 14.217 | 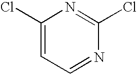 | 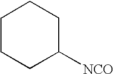 | C |
| 14.218 | 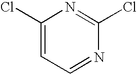 | 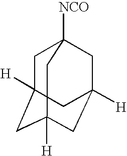 | C |
| 14.219 | 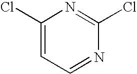 | 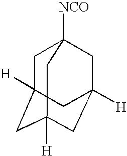 | C |
| 14.220 | 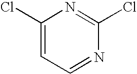 | 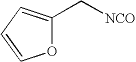 | C |
| 14.221 | 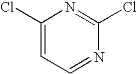 | 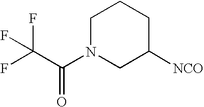 | C |
| 14.222 | 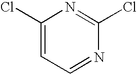 | 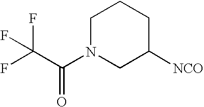 | C |
| 14.223 | 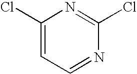 | 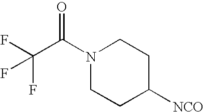 | C |
| 14.224 | 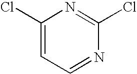 | 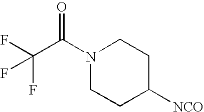 | C |
| 14.225 | 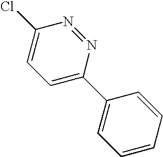 | 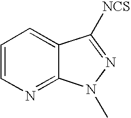 | N |
| 14.226 | 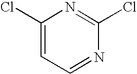 | 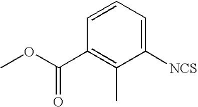 | G |
| 14.227 | 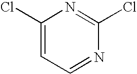 | 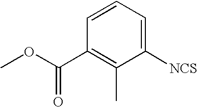 | G |
| 14.228 | 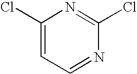 | 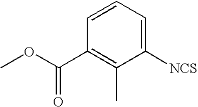 | G |
| 14.229 | 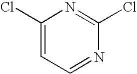 | 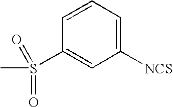 | G |
| 14.230 | 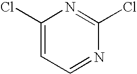 | 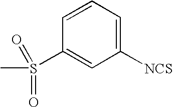 | G |
| 14.231 | 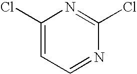 | 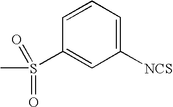 | G |
| 14.232 | 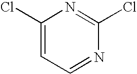 | 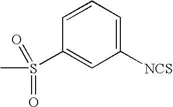 | G |
| 14.233 | 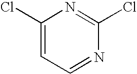 | 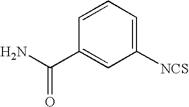 | G |
| 14.234 | 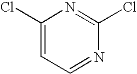 | 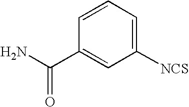 | G |
| 14.235 | 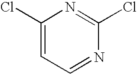 | 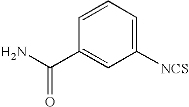 | G |
| 14.236 | 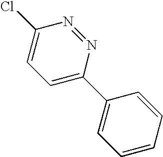 | 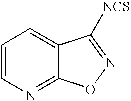 | N |
| 14.237 | 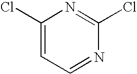 | 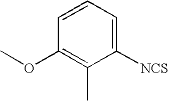 | G |
| 14.238 | 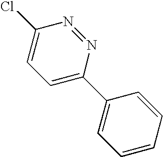 | 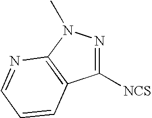 | G |
| 14.239 | 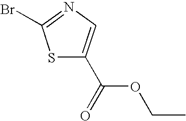 | 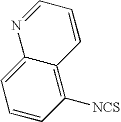 | G |
| 14.240 | 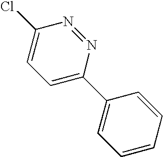 | 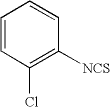 | G |
| 14.241 | 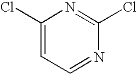 | 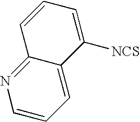 | G |
| 14.242 | 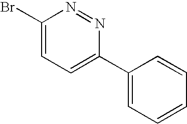 | 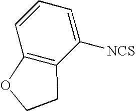 | G |
| 14.243 | 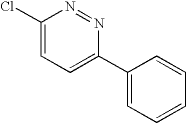 | 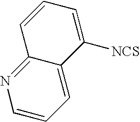 | G |
| 14.244 | 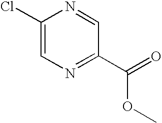 | 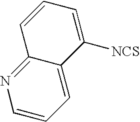 | G |
| 14.245 | 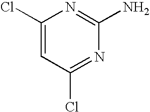 | 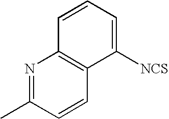 | G |

| Ex # | Product | Rt/min (method) | m/z |
|---|---|---|---|
| 14.1 | 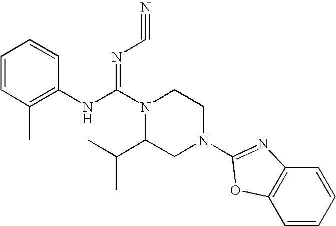 | 1.97 (d) | 403 (M + H)⁺ |
| 14.2 | 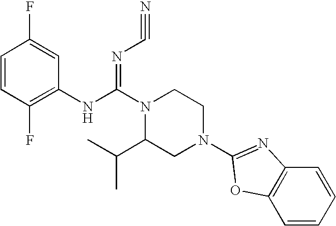 | 1.98 (d) | 425 (M + H)⁺ |
| 14.3 | 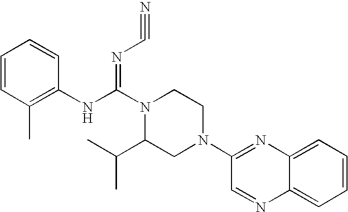 | 1.91 (d) | 412 (M − H)⁻ |
| 14.4 | 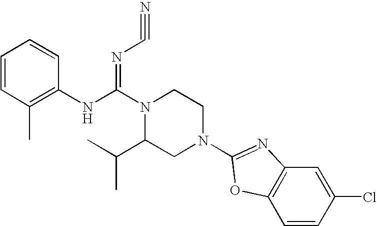 | 2.31 (d) | 437 (M + H)⁺ |
| 14.5 | 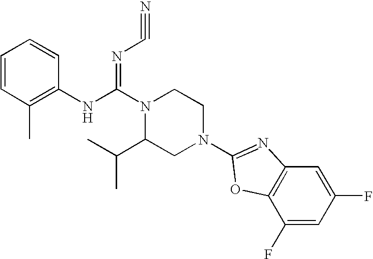 | 2.33 (d) | 439 (M + H)⁺ |
| 14.6 | 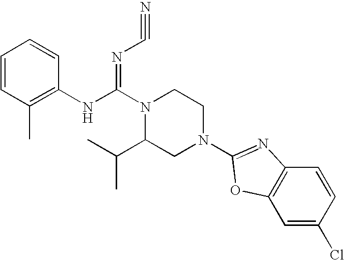 | 2.33 (d) | 437 (M + H)⁺ |
| 14.7 | 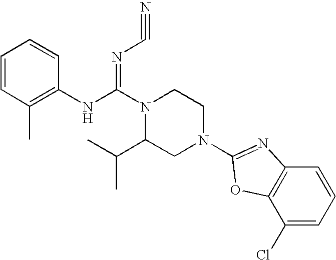 | 2.36 (d) | 437 (M + H)⁺ |
| 14.8 | 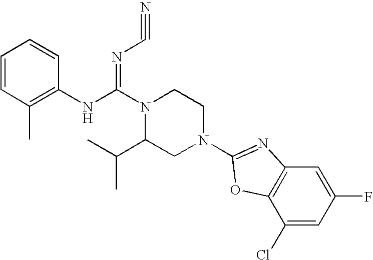 | 2.47 (d) | 455 (M + H)⁺ |
| 14.9 | 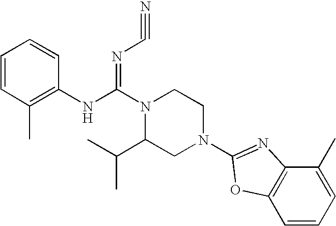 | 2.15 (d) | 417 (M + H)⁺ |
| 14.10 | 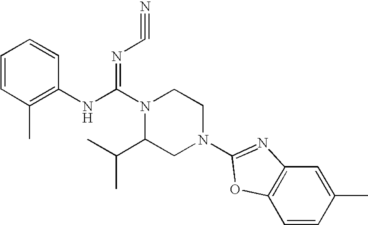 | 2.17 (d) | 417 (M + H)⁺ |
| 14.11 | 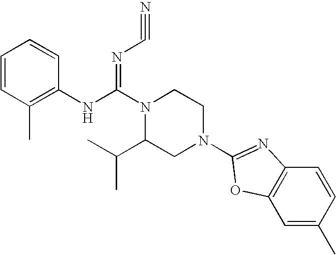 | 2.20 (d) | 417 (M + H)⁺ |
| 14.12 | 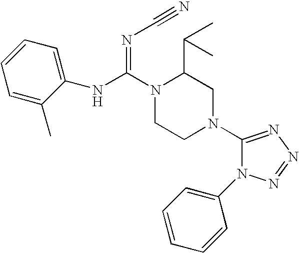 | 1.78 (d) | 430 (M + H)⁺ |
| 14.13 | 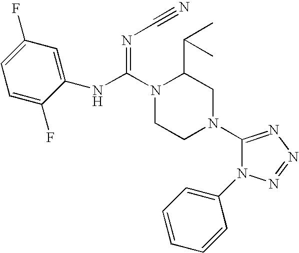 | 1.78 (d) | 452 (M + H)⁺ |
| 14.14 | 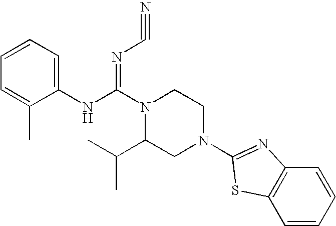 | 2.20 (d) | 417 (M − H)⁻ |
| 14.15 | 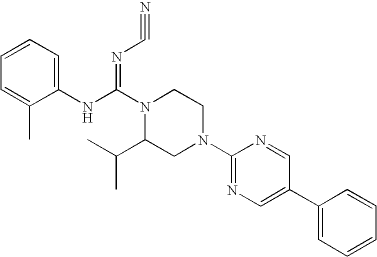 | 2.33 (d) | 440 (M + H)⁺ |
| 14.16 | 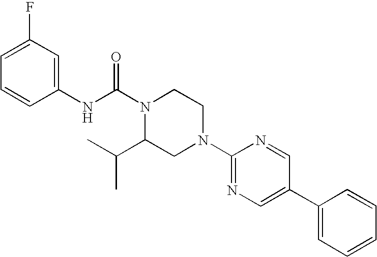 | 2.76 (d) | 420 (M + H)⁺ |
| 14.17 | 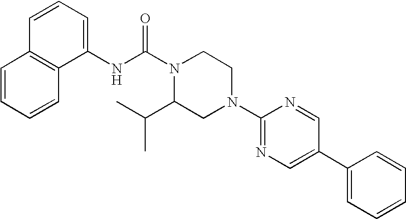 | 2.74 (d) | 452 (M + H)⁺ |
| 14.18 | 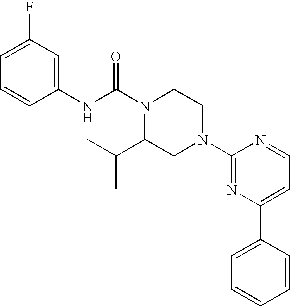 | 2.71 (d) | 420 (M + H)⁺ |
| 14.19 | 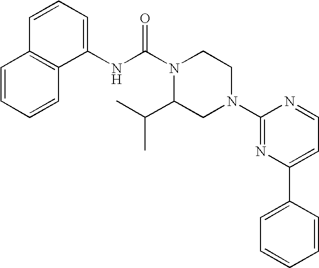 | 2.72 (d) | 452 (M + H)⁺ |
| 14.20 | 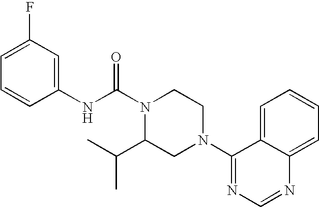 | 2.05 (d) | 394 (M + H)⁺ |
| 14.21 | 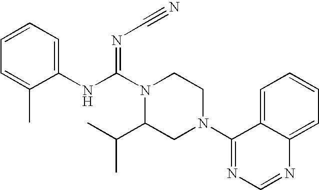 | 1.95 (d) | 412 (M − H)⁻ |
| 14.22 | 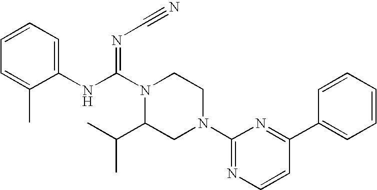 | 2.42 (d) | 438 (M − H)⁻ |
| 14.23 | 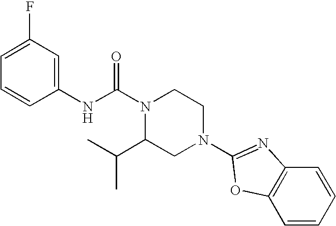 | 2.22 (d) | 381 (M − H)⁻ |
| 14.24 | 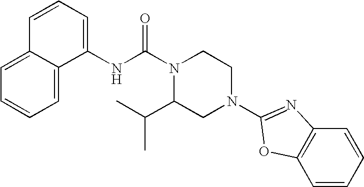 | 2.22 (d) | 413 (M − H)⁻ |
| 14.25 | 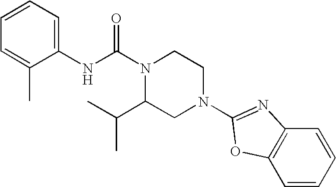 | 2.16 (d) | 379 (M + H)⁺ |
| 14.26 | 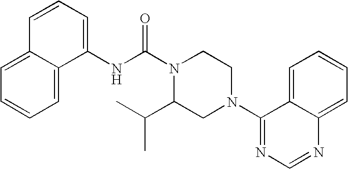 | 1.99 (d) | 424 (M − H)⁻ |
| 14.27 | 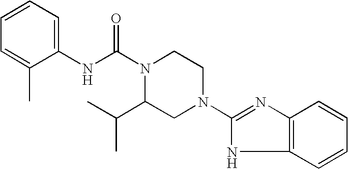 | 1.72 (d) | 376 (M − H)⁻ |
| 14.28 | 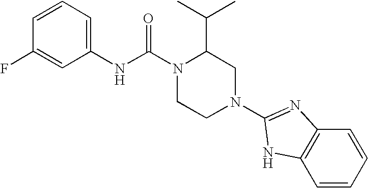 | 1.83 (d) | 380 (M − H)⁻ |
| 14.29 | 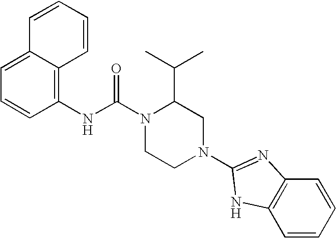 | 1.83 (d) | 414 (M + H)⁺ |
| 14.30 | 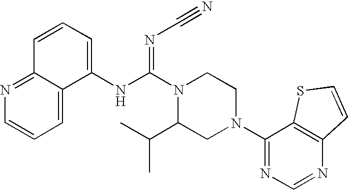 | 2.03 (d) | 455 (M − H)⁻ |
| 14.31 | 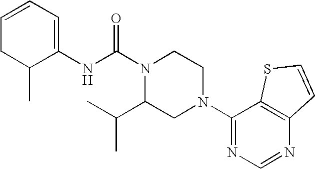 | 2.21 (d) | 394 (M − H)⁻ |
| 14.32 | 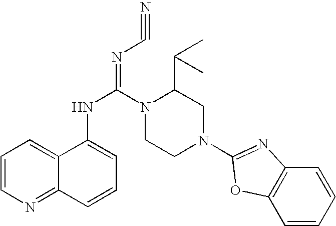 | 1.75 (d) | 438 (M − H)⁻ |
| 14.33 | 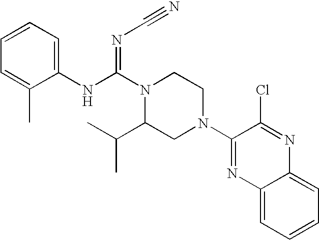 | 2.40 (d) | 446 (M − H)⁻ |
| 14.34 | 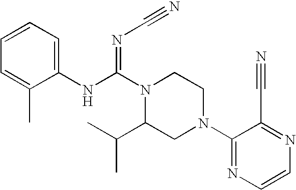 | 1.93 (d) | 387 (M − H)⁻ |
| 14.35 | 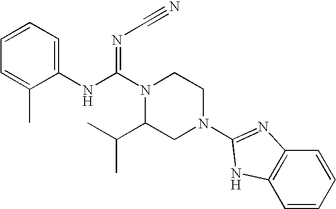 | 2.11 (d) | 400 (M − H)⁻ |
| 14.36 | 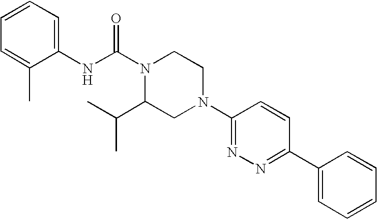 | 1.98 (d) | 414 (M − H)⁻ |
| 14.37 | 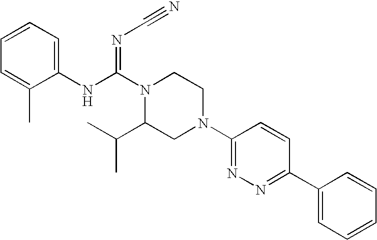 | 2.07 (d) | 440 (M + H)⁺ |
| 14.38 | 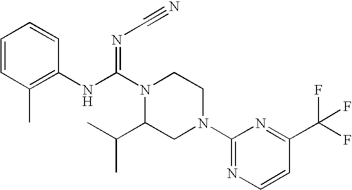 | 2.40 (d) | 430 (M − H)⁻ |
| 14.39 | 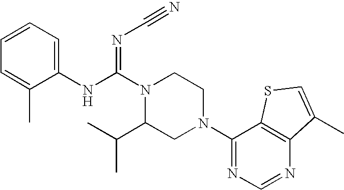 | 1.85 (d) | 434 (M + H)⁺ |
| 14.40 | 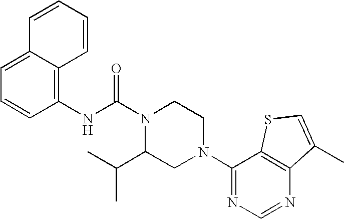 | 2.22 (d) | 444 (M − H)⁻ |
| 14.41 | 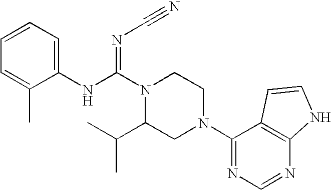 | 1.65 (d) | 401 (M − H)⁻ |
| 14.42 | 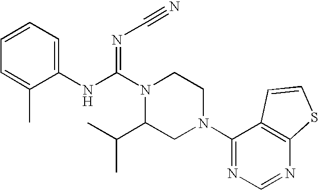 | 1.70 (d) | 418 (M − H)⁻ |
| 14.43 | 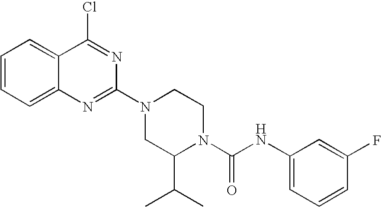 | 2.63 (d) | 426 (M − H)⁻ |
| 14.44 | 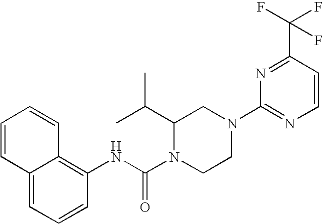 | 2.60 (d) | 442 (M − H)⁻ |
| 14.45 | 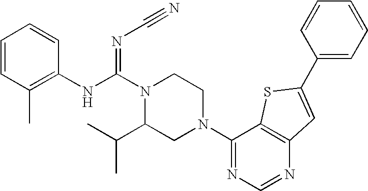 | 2.47 (d) | 494 (M − H)⁻ |
| 14.46 | 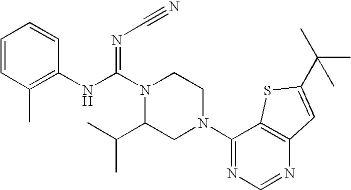 | 2.38 (d) | 476 (M + H)⁺ |
| 14.47 | 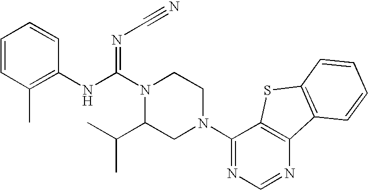 | 2.42 (d) | 468 (M − H)⁻ |
| 14.48 | 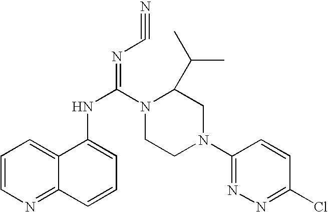 | 1.4 (d) | 435 (M + H)⁺ |
| 14.49 | 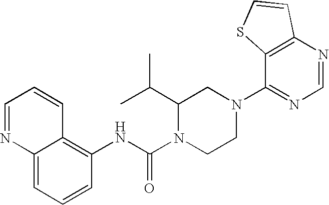 | 1.68 (d) | 433 (M + H)⁺ |
| 14.50 | 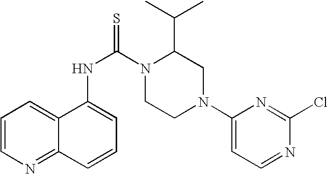 | 1.86 (d) | 425 (M − H)⁻ |
| 14.51 | 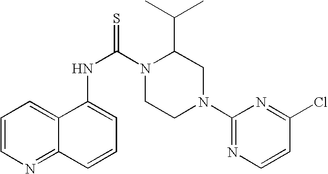 | 2.24 (d) | 425 (M − H)⁻ |
| 14.52 | 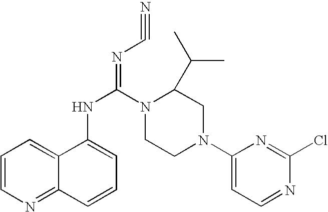 | 1.63 (d) | 435 (M + H)⁺ |
| 14.53 | 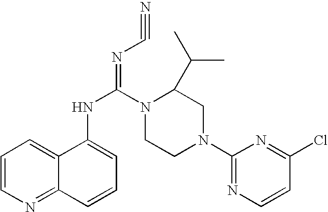 | 2.02 (d) | 435 (M + H)⁺ |
| 14.54 | 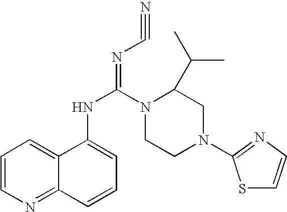 | 1.63 (d) | 406 (M + H)⁺ |
| 14.55 | 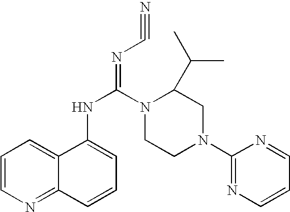 | 1.69 (d) | 401 (M + H)⁺ |
| 14.56 | 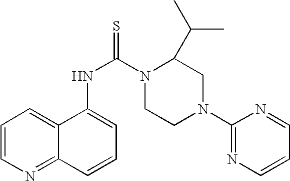 | 1.88 (d) | 391 (M − H)⁻ |
| 14.57 | 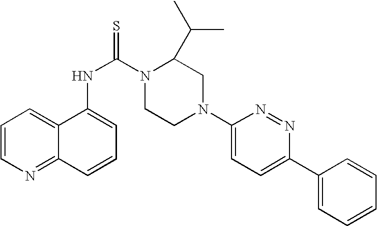 | 2.06 (d) | 467 (M − H)⁻ |
| 14.58 | 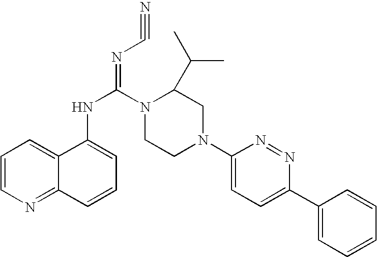 | 1.85 (d) | 475 (M − H)⁻ |
| 14.59 | 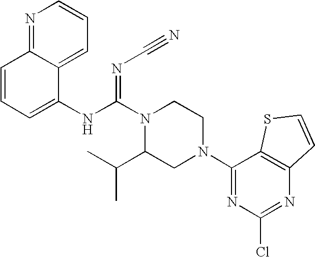 | 1.73 (d) | 491 (M + H)⁺ |
| 14.60 | 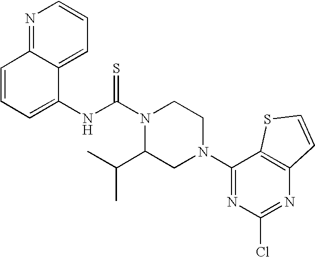 | 1.93 (d) | 481 (M − H)⁻ |
| 14.61 | 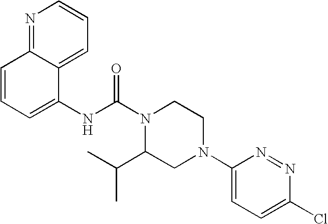 | 1.58 (d) | 411 (M + H)⁺ |
| 14.62 | 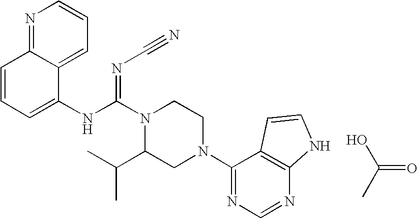 | 1.50 (d) | 440 (M + H)⁺ |
| 14.63 | 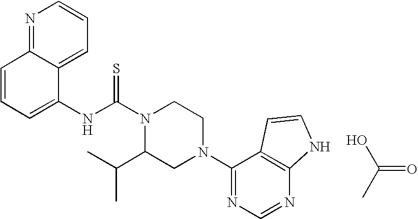 | 1.61 (d) | 430 (M − H)⁻ |
| 14.64 | 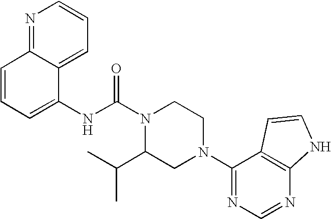 | 1.49 (d) | 416 (M + H)⁺ |
| 14.65 | 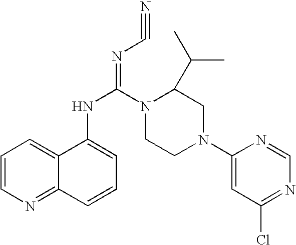 | 1.55 (d) | 433 (M − H)⁻ |
| 14.66 | 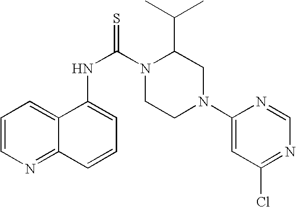 | 1.78 (d) | 425 (M − H)⁻ |
| 14.67 | 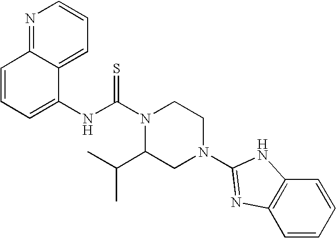 | 1.60 (d) | 429 (M − H)⁻ |
| 14.68 | 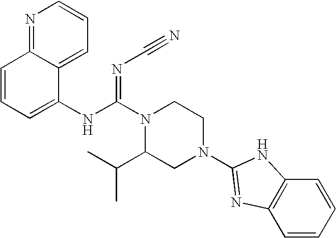 | 1.51 (d) | 439 (M + H)⁺ |
| 14.69 | 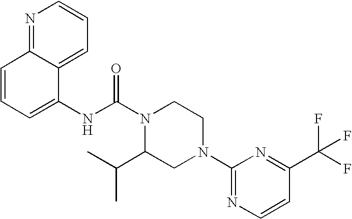 | 2.02 (d) | 442 (M − H)⁻ |
| 14.70 | 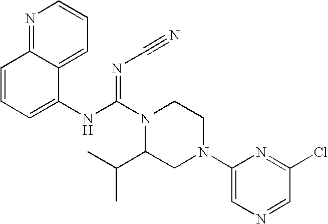 | 1.72 (d) | 443 (M − H)⁻ |
| 14.71 | 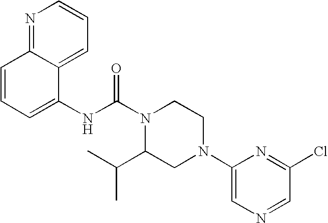 | 1.77 (d) | 409 (M − H)⁻ |
| 14.72 | 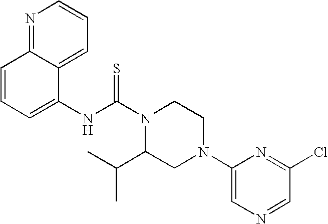 | 1.57 (d) | 425 (M − H)⁻ |
| 14.73 | 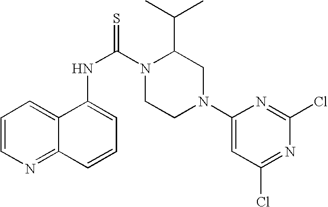 | 2.08 (d) | 459 (M − H)⁻ |
| 14.74 | 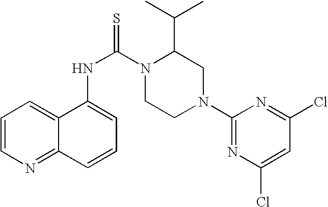 | 2.42 (d) | 459 (M − H)⁻ |
| 14.75 | 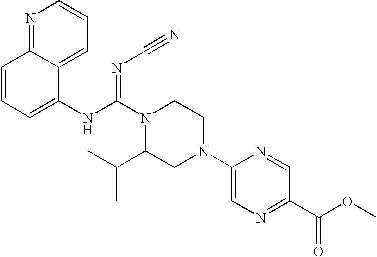 | 1.46 (d) | 457 (M − H)⁻ |
| 14.76 | 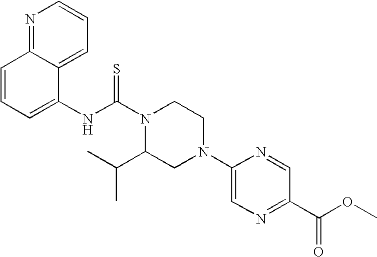 | 1.70 (d) | 449 (M − H)⁻ |
| 14.77 | 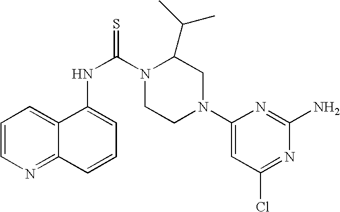 | 1.66 (d) | 440 (M − H)⁻ |
| 14.78 | 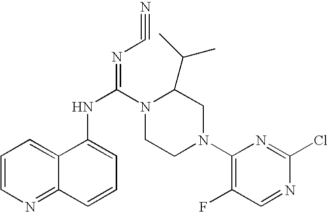 | 1.74 (d) | 451 (M − H)⁻ |
| 14.79 | 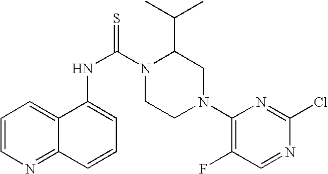 | 1.93 (d) | 443 (M − H)⁻ |
| 14.80 | 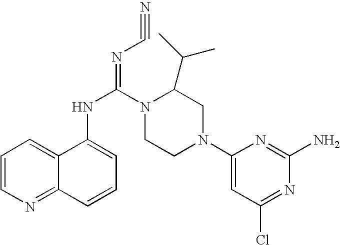 | 1.44 (d) | 448 (M − H)⁻ |
| 14.81 | 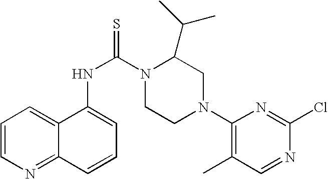 | 1.87 (d) | 439 (M − H)⁻ |
| 14.82 | 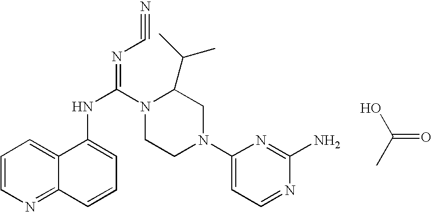 | 0.8 (d) | 416 (M + H)⁺ |
| 14.83 | 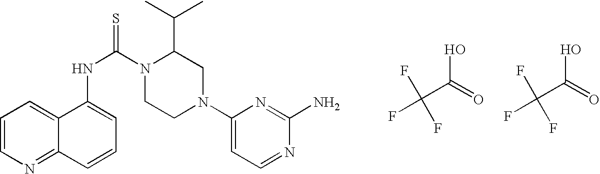 | 1.09 (d) | 406 (M − H)⁻ |
| 14.84 | 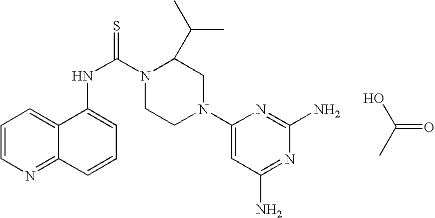 | 1.15 (d) | 421 (M − H)⁻ |
| 14.45 | 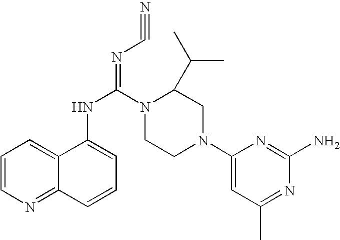 | 1.68 (a) | 428 (M − H)⁻ |
| 14.86 | 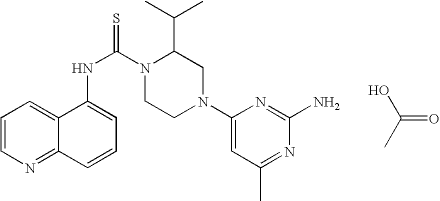 | 1.22 (a) | 420 (M − H)⁻ |
| 14.87 | 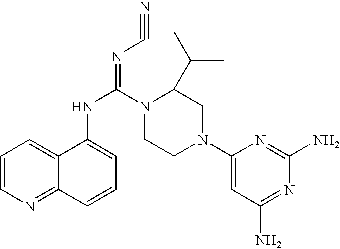 | 0.88 (d) | 429 (M − H)⁻ |
| 14.88 | 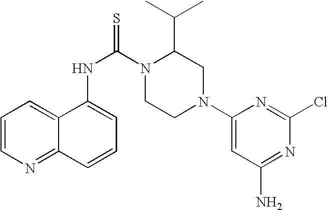 | 1.85 (d) | 440 (M − H)⁻ |
| 14.89 | 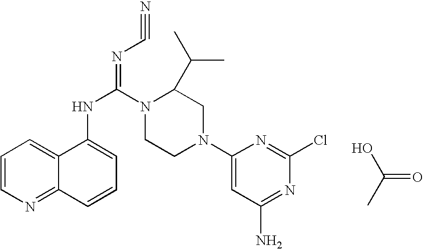 | 1.68 (d) | 448 (M − H)⁻ |
| 14.90 | 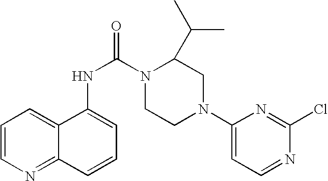 | 1.62 (d) | 409 (M − H)⁻ |
| 14.91 | 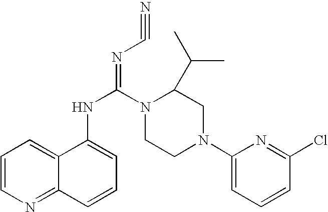 | 1.99 (d) | 432 (M − H)⁻ |
| 14.92 | 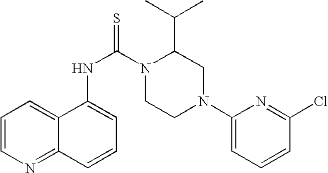 | 2.19 (d) | 424 (M − H)⁻ |
| 14.93 | 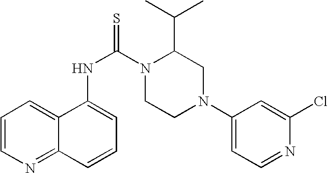 | 1.77 (d) | 424 (M − H)⁻ |
| 14.94 | 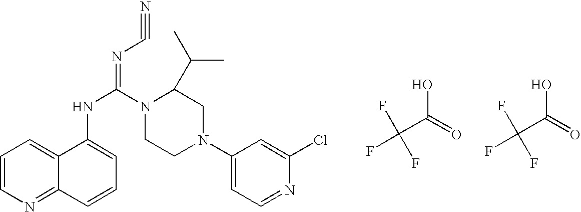 | 1.57 (d) | 432 (M − H)⁻ |
| 14.95 | 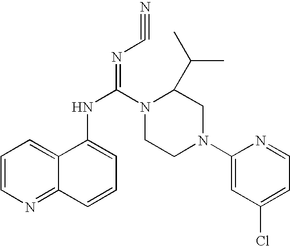 | 1.92 (d) | 432 (M − H)⁻ |
| 14.96 | 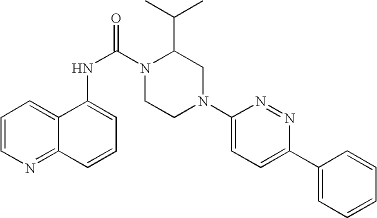 | 1.77 (d) | 451 (M − H)⁻ |
| 14.97 | 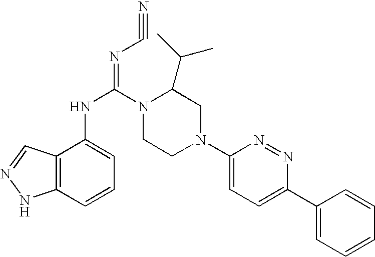 | 1.77 (d) | 464 (M − H)⁻ |
| 14.98 | 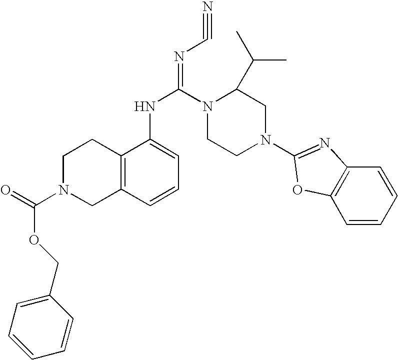 | 2.19 (d) | 578 (M + H)⁺ |
| 14.99 | 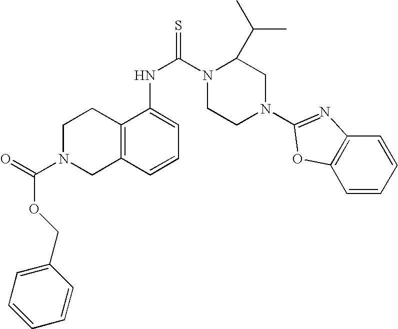 | 2.39 (d) | 570 (M + H)⁺ |
| 14.100 | 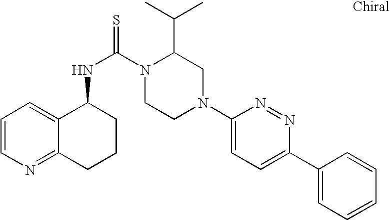 | 2.09 (d) | 471 (M − H)⁻ |
| 14.101 | 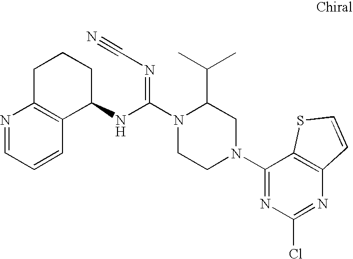 | 1.87 (d) | 495 (M + H)⁺ |
| 14.102 | 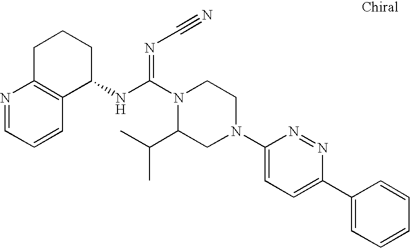 | 1.60 (d) | 479 (M − H)⁻ |
| 14.103 | 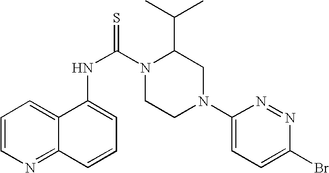 | 1.76 (d) | 469 (M − H)⁻ |
| 14.104 | 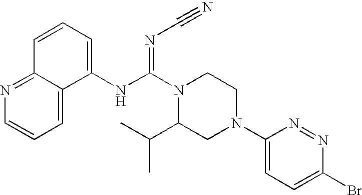 | 1.54 (d) | 477 (M − H)⁻ |
| 14.105 | 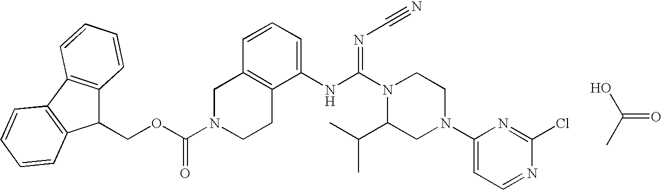 | 2.25 (d) | 659 (M − H)⁻ |
| 14.106 | 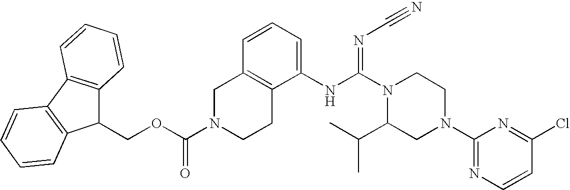 | 2.61 (d) | 659 (M − H)⁻ |
| 14.107 | 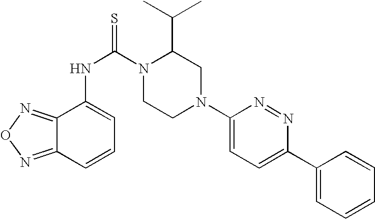 | 2.26 (d) | 458 (M − H)⁻ |
| 14.108 | 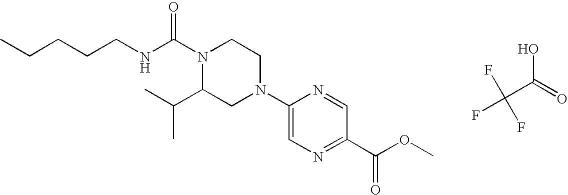 | 1.85 (j) | 377 (M + H)⁺ |
| 14.109 | 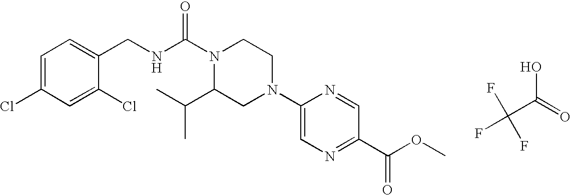 | 2.08 (j) | 465 (M + H)⁺ |
| 14.110 | 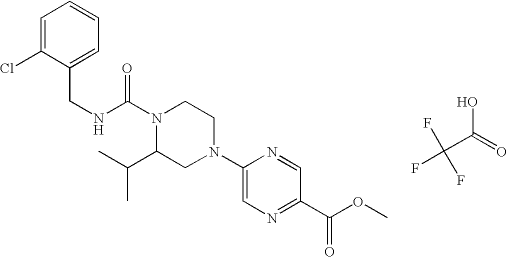 | 1.88 (j) | 431 (M + H)⁺ |
| 14.111 | 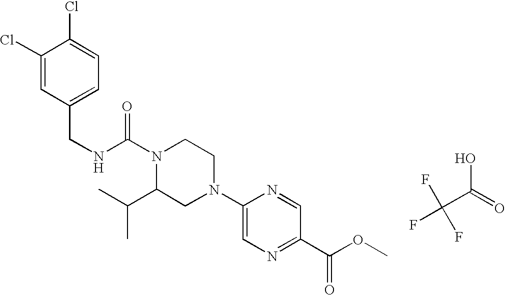 | 2.08 (j) | 465 (M + H)⁺ |
| 14.112 | 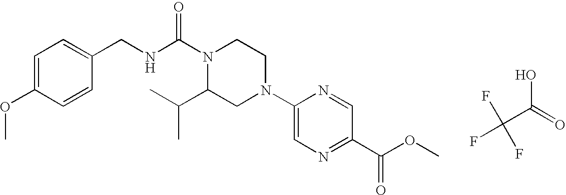 | 1.73 (j) | 427 (M + H)⁺ |
| 14.113 | 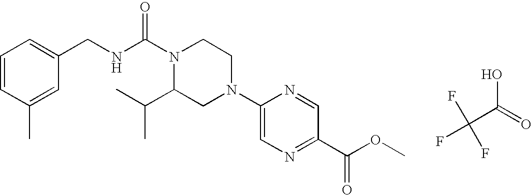 | 1.87 (j) | 411 (M + H)⁺ |
| 14.114 | 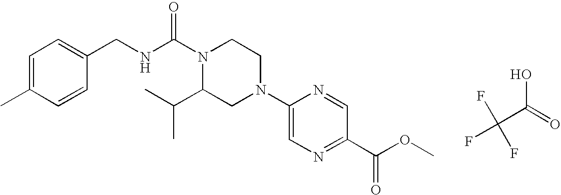 | 1.87 (j) | 411 (M + H)⁺ |
| 14.115 | 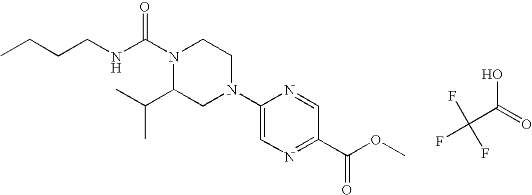 | 1.68 (j) | 363 (M + H)⁺ |
| 14.116 | 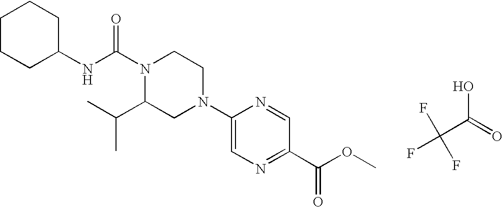 | 1.84 (j) | 389 (M + H)⁺ |
| 14.117 | 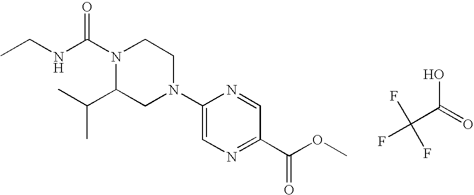 | 1.35 (j) | 335 (M + H)⁺ |
| 14.118 | 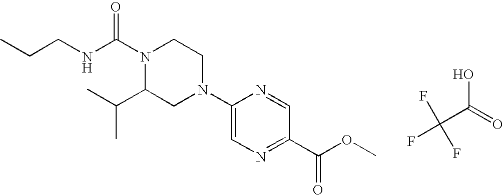 | 1.49 (j) | 349 (M + H)⁺ |
| 14.119 | 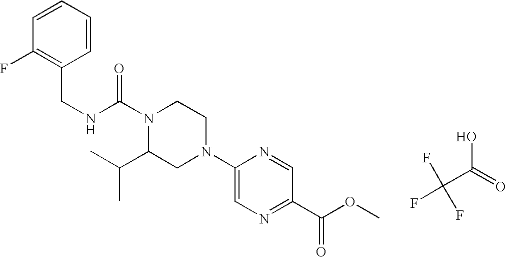 | 1.78 (j) | 415 (M + H)⁺ |
| 14.120 | 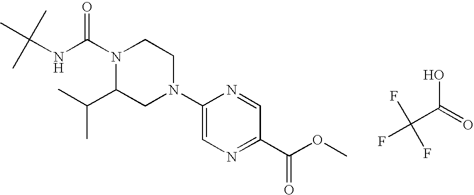 | 1.72 (j) | 363 (M + H)⁺ |
| 14.121 | 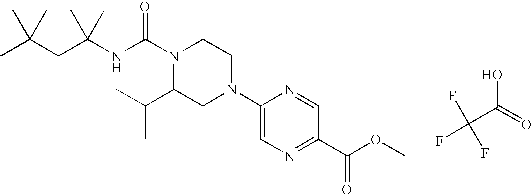 | 2.25 (j) | 419 (M + H)⁺ |
| 14.122 | 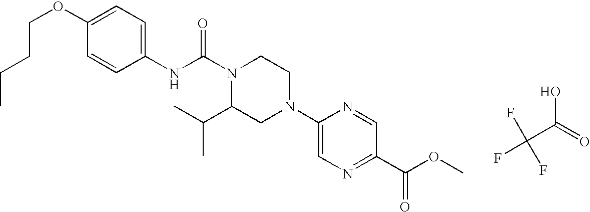 | 2.18 (j) | 455 (M + H)⁺ |
| 14.123 | 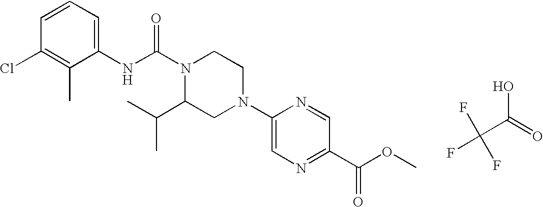 | 1.95 (j) | 431 (M + H)⁺ |
| 14.124 | 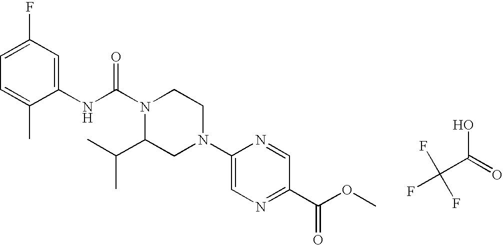 | 1.87 (j) | 415 (M + H)⁺ |
| 14.125 | 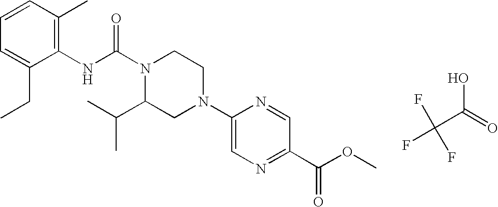 | 1.9 (j) | 425 (M + H)⁺ |
| 14.126 | 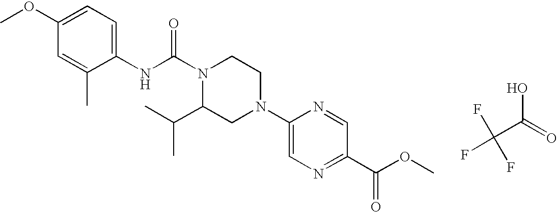 | 1.73 (j) | 427 (M + H)⁺ |
| 14.127 | 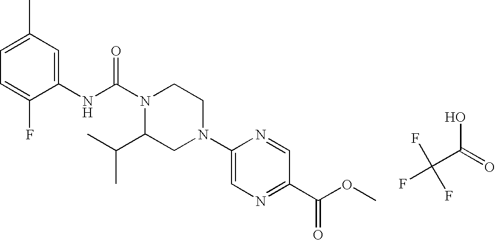 | 1.88 (j) | 415 (M + H)⁺ |
| 14.128 | 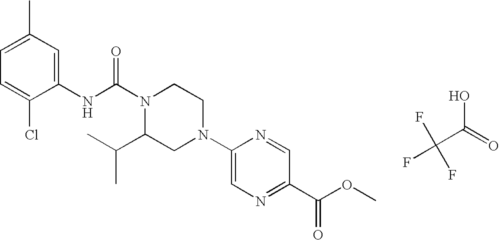 | 2.04 (j) | 431 (M + H)⁺ |
| 14.129 | 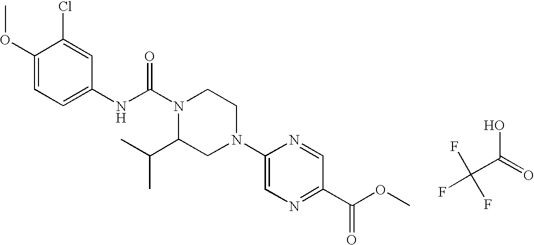 | 1.9 (j) | 447 (M + H)⁺ |
| 14.130 | 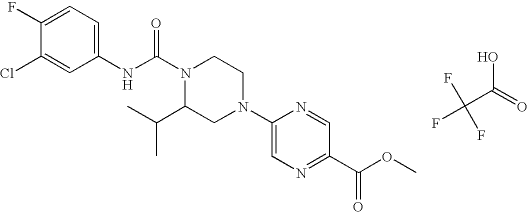 | 2.05 (j) | 435 (M + H)⁺ |
| 14.131 | 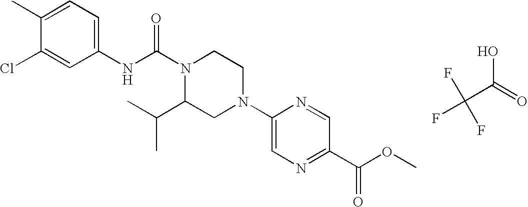 | 2.11 (j) | 431 (M + H)⁺ |
| 14.132 | 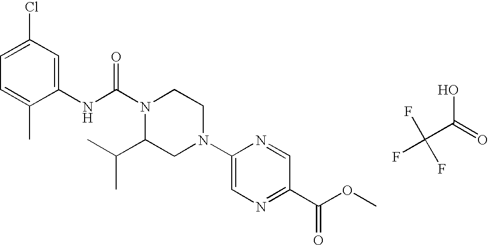 | 2.00 (j) | 431 (M + H)⁺ |
| 14.133 | 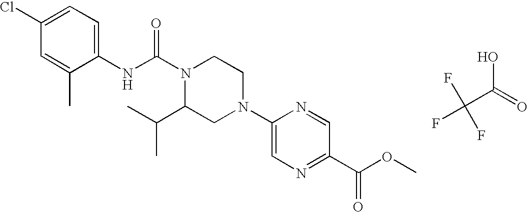 | 1.98 (j) | 431 (M + H)⁺ |
| 14.134 | 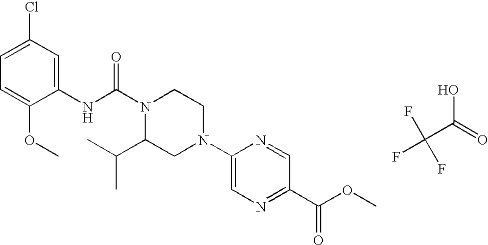 | 2.09 (j) | 447 (M + H)⁺ |
| 14.135 | 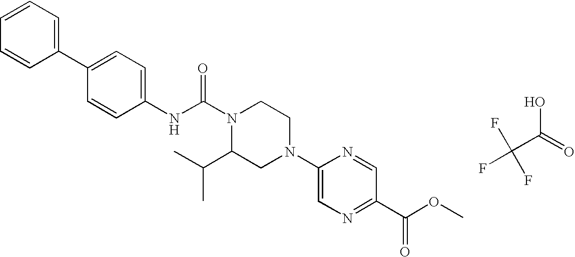 | 2.19 (j) | 459 (M + H)⁺ |
| 14.136 | 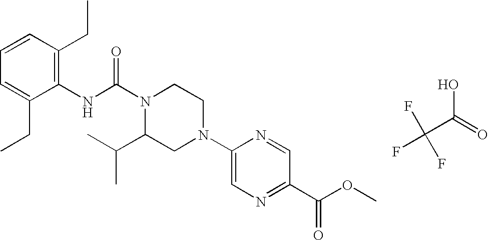 | 2.03 (j) | 439 (M + H)⁺ |
| 14.137 | 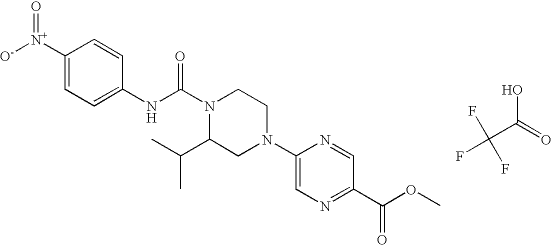 | 1.88 (j) | 428 (M + H)⁺ |
| 14.138 | 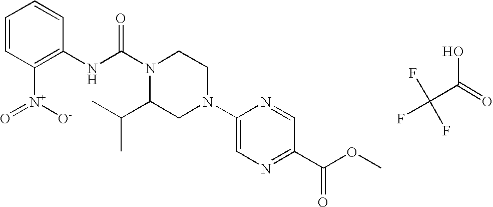 | 1.97 (j) | 428 (M + H)⁺ |
| 14.139 | 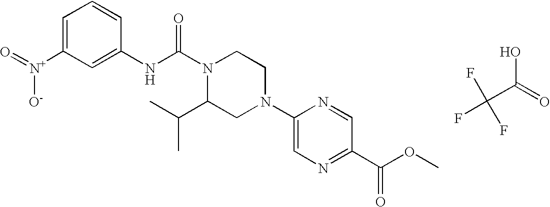 | 1.89 (j) | 428 (M + H)⁺ |
| 14.140 | 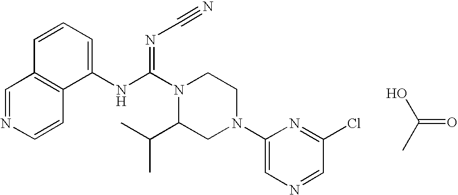 | 1.55 (d) | 433 (M − H)⁻ |
| 14.141 | 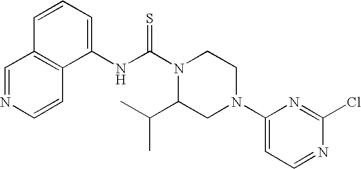 | 1.70 (d) | 425 (M − H)⁻ |
| 14.142 | 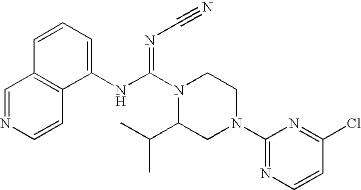 | 1.80 (d) | 433 (M − H)⁻ |
| 14.143 | 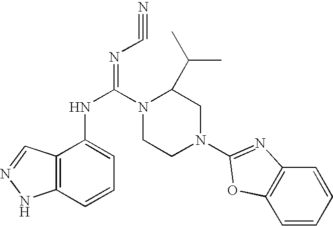 | 1.64 (d) | 429 (M + H)⁺ |
| 14.144 | 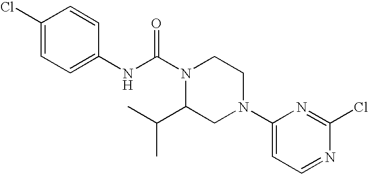 | 2.60 (b) | 394 (M + H)⁺ |
| 14.145 | 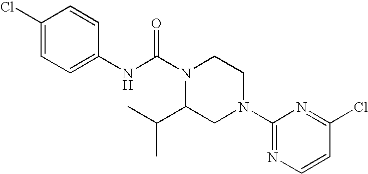 | 3.02 (b) | 395 (M + H)⁺ |
| 14.146 | 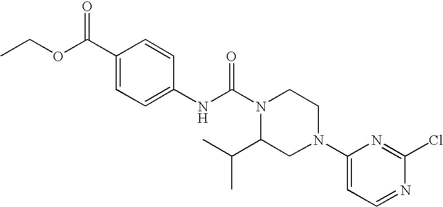 | 2.56 (b) | 432 (M + H)⁺ |
| 14.147 | 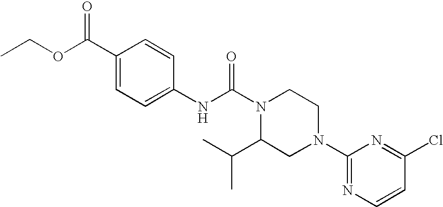 | 3.00 (b) | 432 (M + H)⁺ |
| 14.148 | 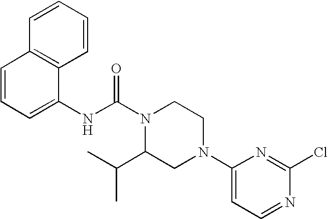 | 2.50 (b) | 410 (M + H)⁺ |
| 14.149 | 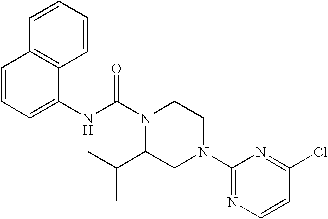 | 2.93 (b) | 410 (M + H)⁺ |
| 14.150 | 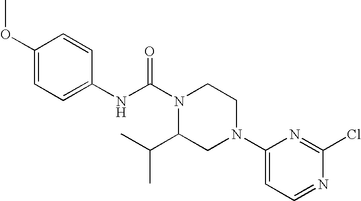 | 2.29 (b) | 390 (M + H)⁺ |
| 14.151 | 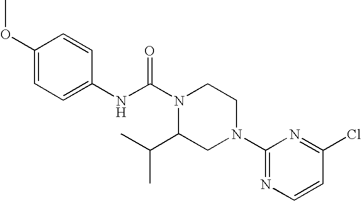 | 2.72 (b) | 390 (M + H)⁺ |
| 14.152 | 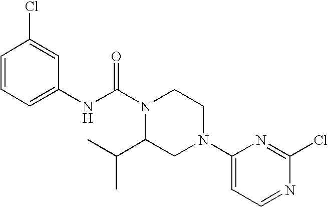 | 2.62 (b) | 394 (M + H)⁺ |
| 14.153 | 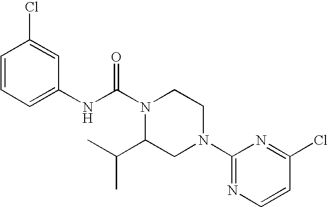 | 3.05 (b) | 394 (M + H)⁺ |
| 14.154 | 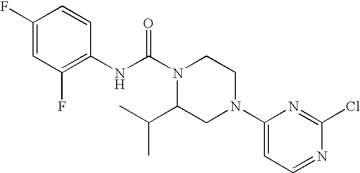 | 2.38 (b) | 396 (M + H)⁺ |
| 14.155 | 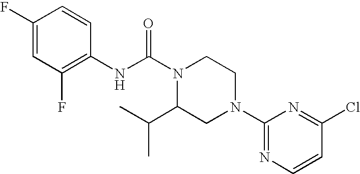 | 2.83 (b) | 396 (M + H)⁺ |
| 14.156 | 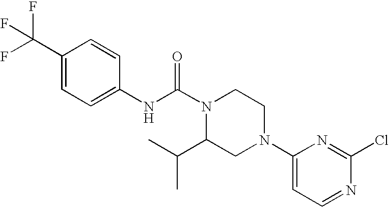 | 2.73 (b) | 428 (M + H)⁺ |
| 14.157 | 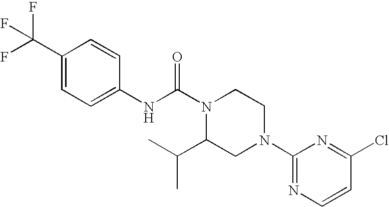 | 3.13 (b) | 428 (M + H)⁺ |
| 14.158 | 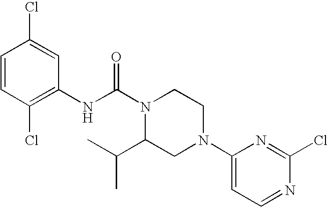 | 2.87 (b) | 428 (M + H)⁺ |
| 14.159 | 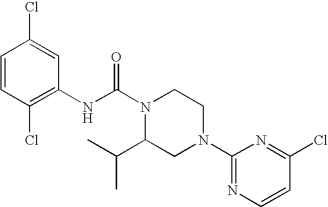 | 3.34 (b) | 428 (M + H)⁺ |
| 14.160 | 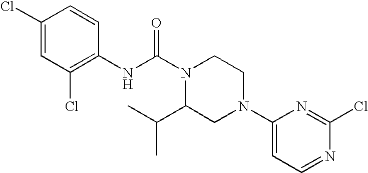 | 2.81 (b) | 428 (M + H)⁺ |
| 14.161 | 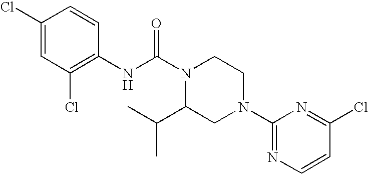 | 3.29 (b) | 428 (M + H)⁺ |
| 14.162 | 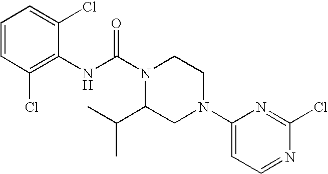 | 4.42 (b) | 428 (M + H)⁺ |
| 14.163 | 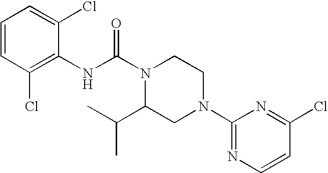 | 2.88 (b) | 428 (M + H)⁺ |
| 14.164 | 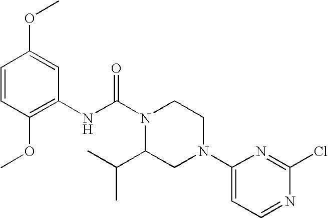 | 2.50 (b) | 420 (M + H)⁺ |
| 14.165 | 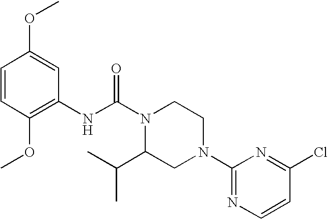 | 2.99 (b) | 420 (M + H)⁺ |
| 14.166 | 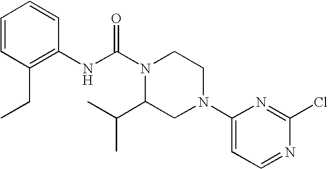 | 2.49 (b) | 388 (M + H)⁺ |
| 14.167 | 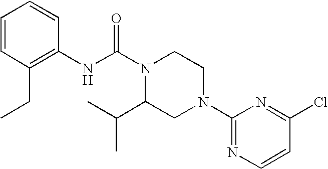 | 2.94 (b) | 388 (M + H)⁺ |
| 14.168 | 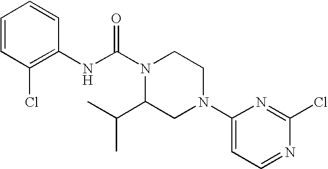 | 2.55 (b) | 394 (M + H)⁺ |
| 14.169 | 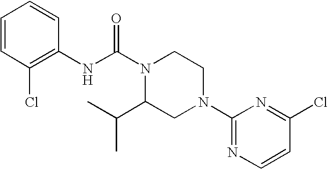 | 3.06 (b) | 394 (M + H)⁺ |
| 14.170 | 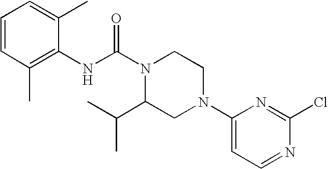 | 2.39 (b) | 388 (M + H)⁺ |
| 14.171 | 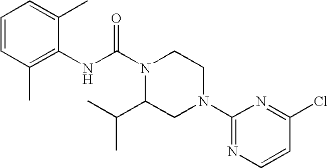 | 2.84 (b) | 388 (M + H)⁺ |
| 14.172 | 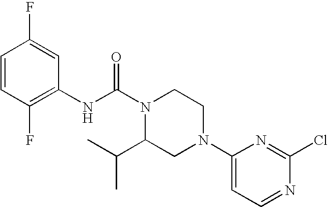 | 2.50 (b) | 396 (M + H)⁺ |
| 14.173 | 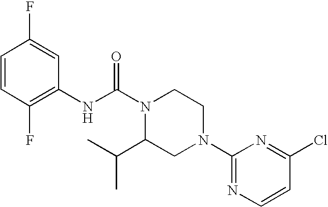 | 2.97 (b) | 396 (M + H)⁺ |
| 14.174 | 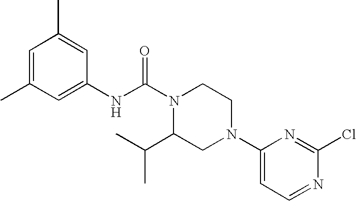 | 2.63 (b) | 387 (M + H)⁺ |
| 14.175 | 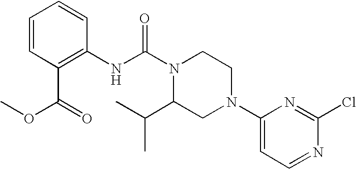 | 2.93 (b) | 418 (M + H)⁺ |
| 14.176 | 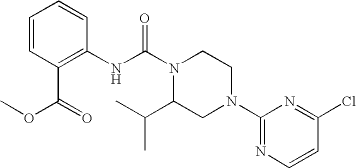 | 3.21 (b) | 418 (M + H)⁺ |
| 14.177 | 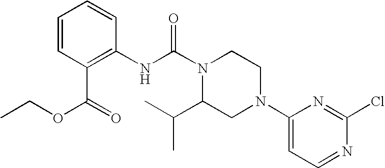 | 3.06 (b) | 432 (M + H)⁺ |
| 14.178 | 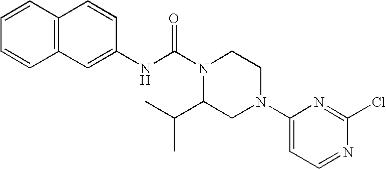 | 2.85 (b) | 410 (M + H)⁺ |
| 14.179 | 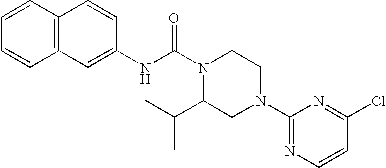 | 3.05 (b) | 410 (M + H)⁺ |
| 14.180 | 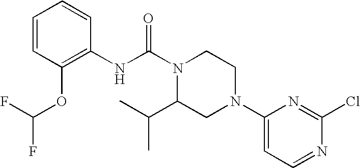 | 2.54 (b) | 426 (M + H)⁺ |
| 14.181 | 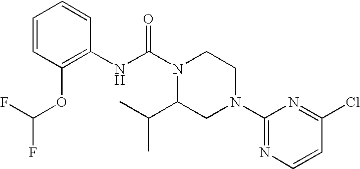 | 2.88 (b) | 426 (M + H)⁺ |
| 14.182 | 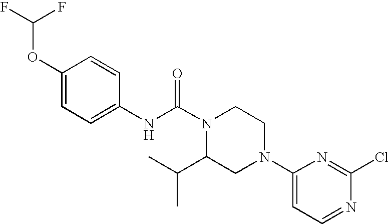 | 2.56 (b) | 426 (M + H)⁺ |
| 14.183 | 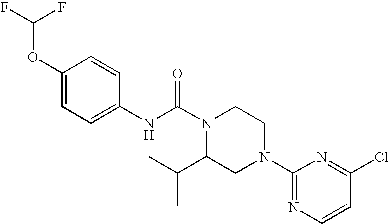 | 2.85 (b) | 426 (M + H)⁺ |
| 14.184 | 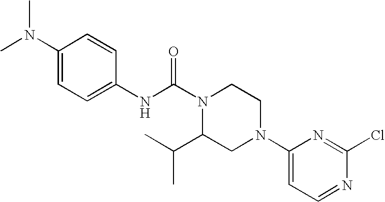 | 2.52 (b) | 403 (M + H)⁺ |
| 14.185 | 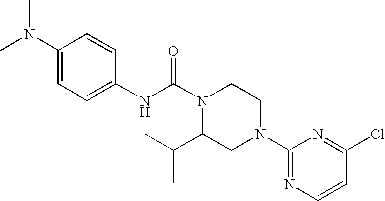 | 2.86 (b) | 413 (M + H)⁺ |
| 14.186 | 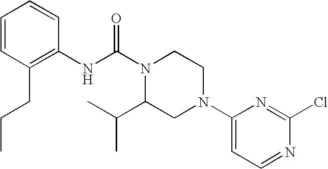 | 2.82 (b) | 402 (M + H)⁺ |
| 14.187 | 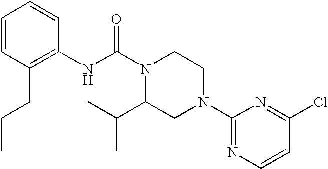 | 3.14 (b) | 402 (M + H)⁺ |
| 14.188 | 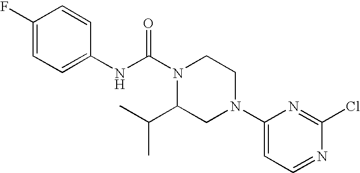 | 2.65 (b) | 378 (M + H)⁺ |
| 14.189 | 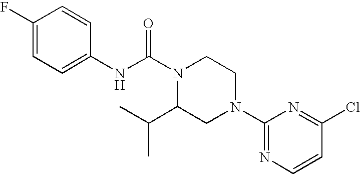 | 2.95 (b) | 378 (M + H)⁺ |
| 14.190 | 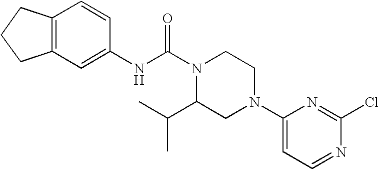 | 2.67 (b) | 400 (M + H)⁺ |
| 14.191 | 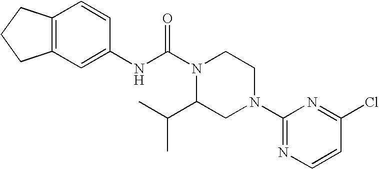 | 3.19 (b) | 400 (M + H)⁺ |
| 14.192 | 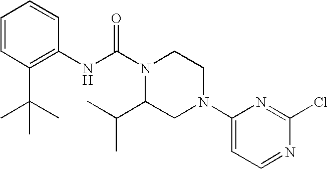 | 2.80 (b) | 416 (M + H)⁺ |
| 14.193 | 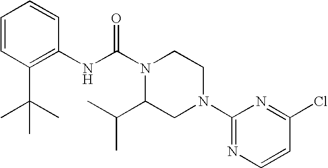 | 3.13 (b) | 416 (M + H)⁺ |
| 14.194 | 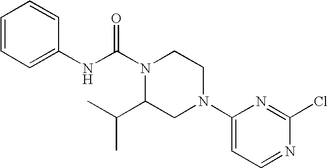 | 2.62 (b) | 360 (M + H)⁺ |
| 14.195 | 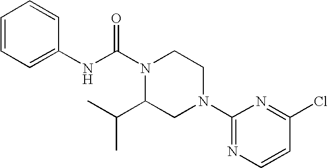 | 2.88 (b) | 360 (M + H)⁺ |
| 14.196 | 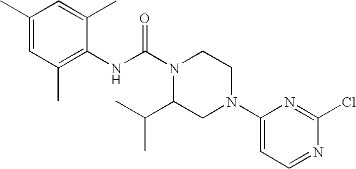 | 2.73 (b) | 402 (M + H)⁺ |
| 14.197 | 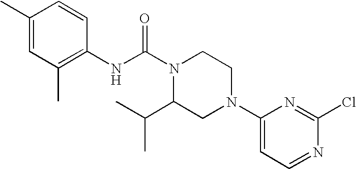 | 2.99 (b) | 388 (M + H)⁺ |
| 14.198 | 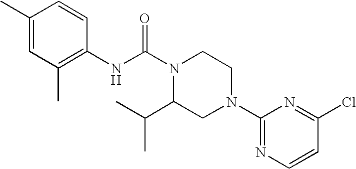 | 2.67 (b) | 388 (M + H)⁺ |
| 14.199 | 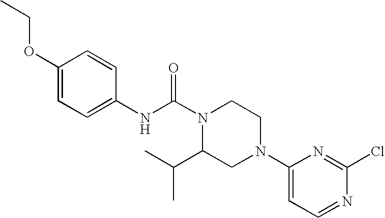 | 2.66 (b) | 404 (M + H)⁺ |
| 14.200 | 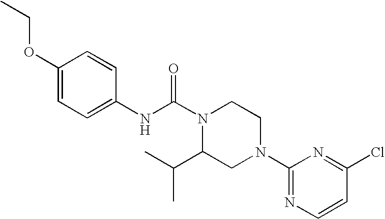 | 2.95 (b) | 404 (M + H)⁺ |
| 14.201 | 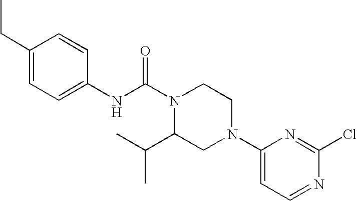 | 2.85 (b) | 388 (M + H)⁺ |
| 14.202 | 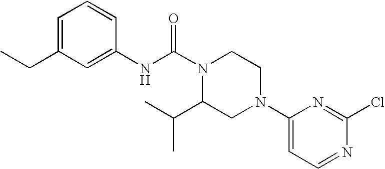 | 2.94 (b) | 388 (M + H)⁺ |
| 14.203 | 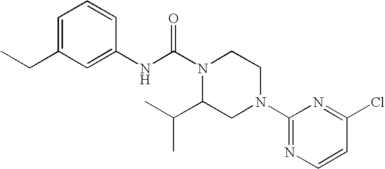 | 3.12 (b) | 388 (M + H)⁺ |
| 14.204 | 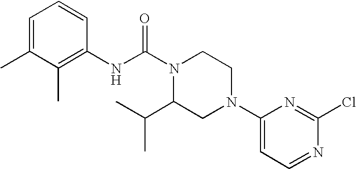 | 2.68 (b) | 388 (M + H)⁺ |
| 14.205 | 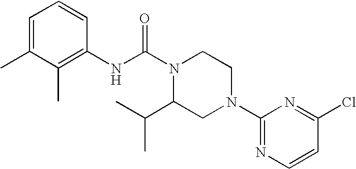 | 2.93 (b) | 388 (M + H)⁺ |
| 14.206 | 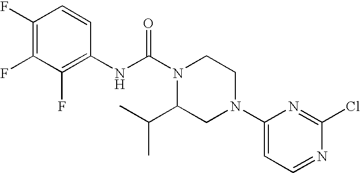 | 2.70 (b) | 414 (M + H)⁺ |
| 14.207 | 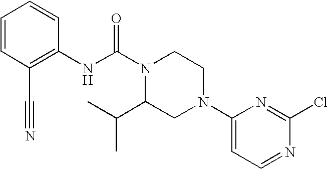 | 2.56 (b) | 383 (M + H)⁺ |
| 14.208 | 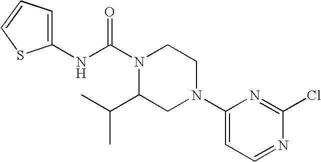 | 2.55 (b) | 366 (M + H)⁺ |
| 14.209 | 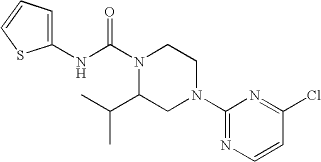 | 2.90 (b) | 366 (M + H)⁺ |
| 14.210 | 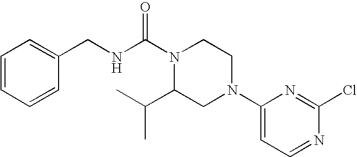 | 2.53 (b) | 374 (M + H)⁺ |
| 14.211 | 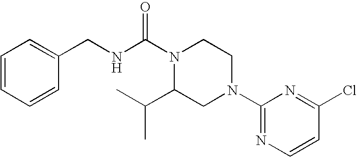 | 2.89 (b) | 374 (M + H)⁺ |
| 14.212 | 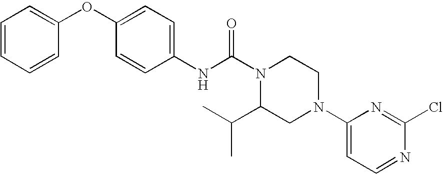 | 2.85 (b) | 452 (M + H)⁺ |
| 14.213 | 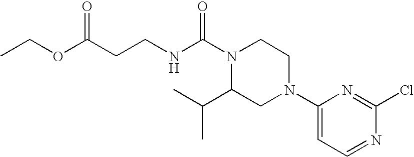 | 2.36 (b) | 384 (M + H)⁺ |
| 14.214 | 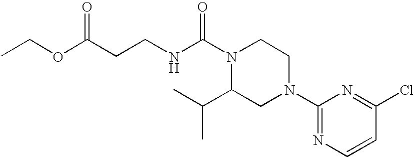 | 2.68 (b) | 384 (M + H)⁺ |
| 14.215 | 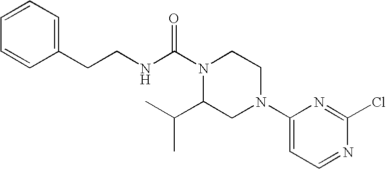 | 2.60 (b) | 388 (M + H)⁺ |
| 14.216 | 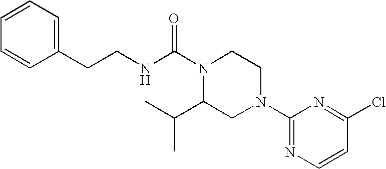 | 2.94 (b) | 388 (M + H)⁺ |
| 14.217 | 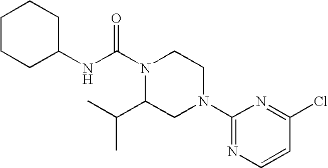 | 2.53 (b) | 356 (M + H)⁺ |
| 14.218 | 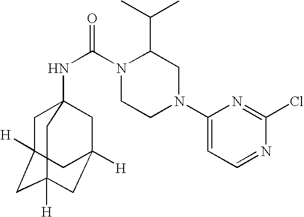 | 2.90 (b) | 418 (M + H)⁺ |
| 14.219 | 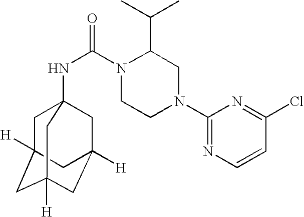 | 3.23 (b) | 418 (M + H)⁺ |
| 14.220 | 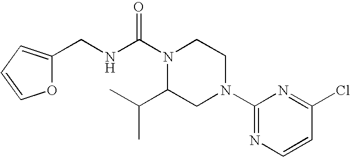 | 2.24 (b) | 364 (M + H)⁺ |
| 14.221 | 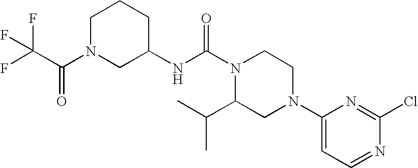 | 2.41 (b) | 463 (M + H)⁺ |
| 14.222 | 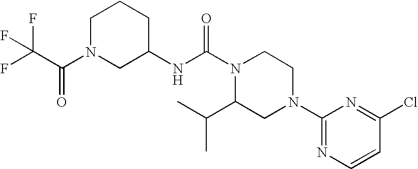 | 2.69 (b) | 463 (M + H)⁺ |
| 14.223 | 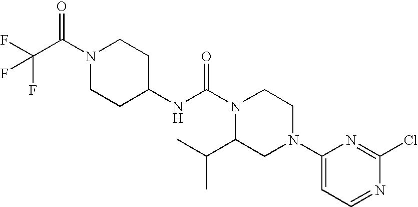 | 2.42 (b) | 463 (M + H)⁺ |
| 14.224 | 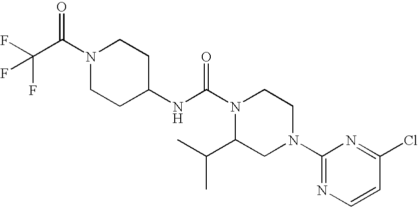 | 2.59 (b) | 463 (M + H)⁺ |
| 14.225 | 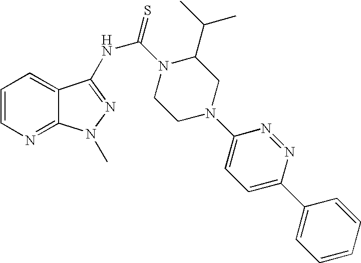 | 1.92 (b) | 473 (M + H)⁺ |
| 14.226 | 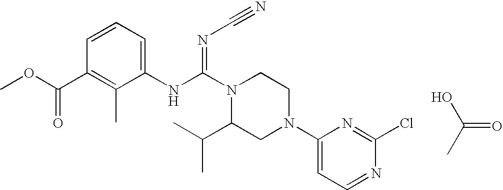 | 1.76 (d) | 454 (M − H)⁻ |
| 14.227 | 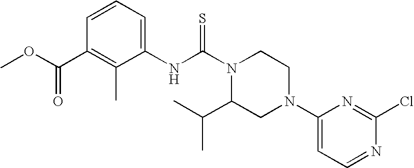 | 2.01 (d) | 446 (M − H)⁻ |
| 14.228 | 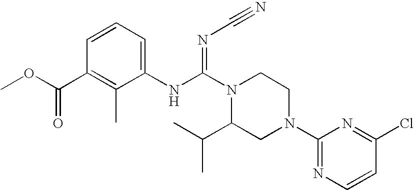 | 2.11 (d) | 454 (M − H)⁻ |
| 14.229 | 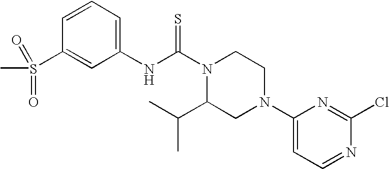 | 1.79 (d) | 452 (M − H)⁻ |
| 14.230 | 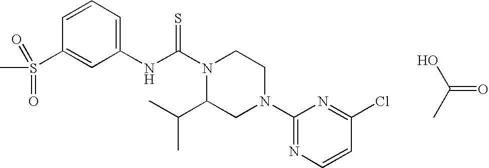 | 2.16 (d) | 452 (M − H)⁻ |
| 14.231 | 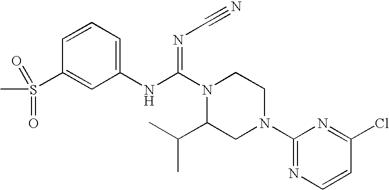 | 1.89 (d) | 460 (M − H)⁻ |
| 14.232 | 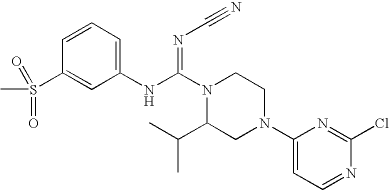 | 1.55 (d) | 460 (M − H)⁻ |
| 14.233 | 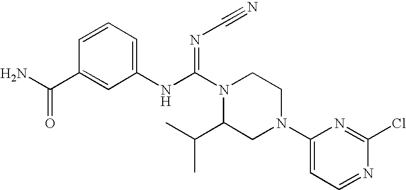 | 1.38 (d) | 425 (M − H)⁻ |
| 14.234 | 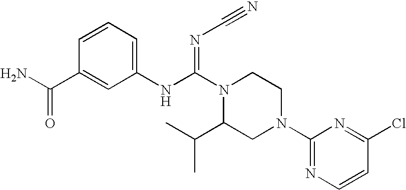 | 1.66 (d) | 425 (M − H)⁻ |
| 14.235 | 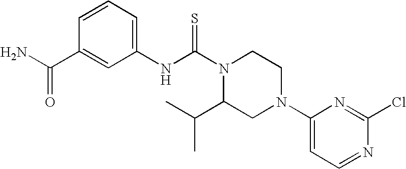 | 1.50 (d) | 417 (M − H)⁻ |
| 14.236 | 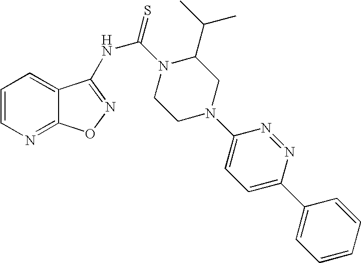 | 1.77 (d) | 460 (M + H)⁺ |
| 14.237 | 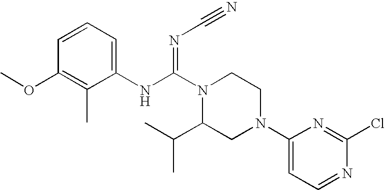 | 1.85 (d) | 426 (M − H)⁻ |
| 14.238 | 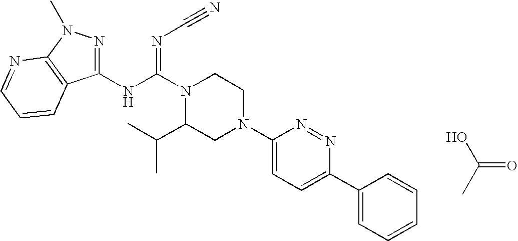 | 2.18 (d) | 481 (M + H)⁺ |
| 14.239 | 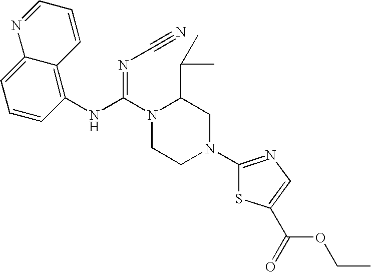 | 1.75 (d) | 478 (M + H)⁺ |
| 14.240 | 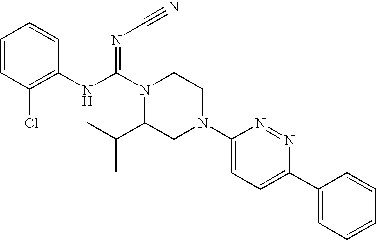 | 2.06 (d) | 460 (M + H)⁺ |
| 14.241 | 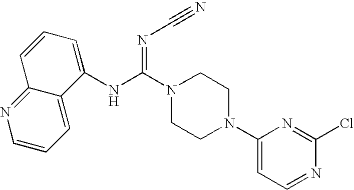 | 1.47 (b) | 391 (M − H)⁻ |
| 14.242 | 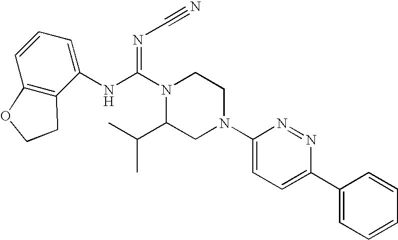 | 1.91 (d) | 468 (M + H)⁺ |
| 14.243 | 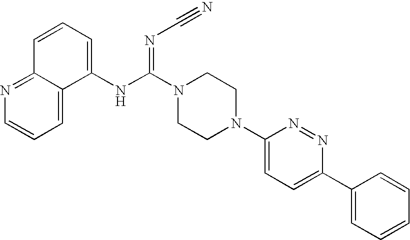 | 1.43 (d) | 435 (M + H)⁺ |
| 14.244 | 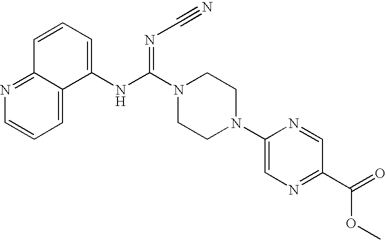 | 0.96 (d) | 417 (M + H)⁺ |
| 14.245 | 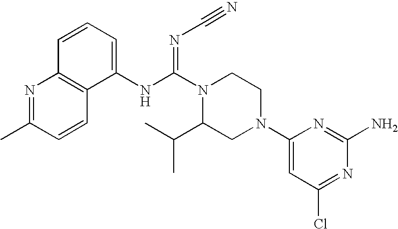 | 1.64 (b) | 464 (M + H)⁺ |

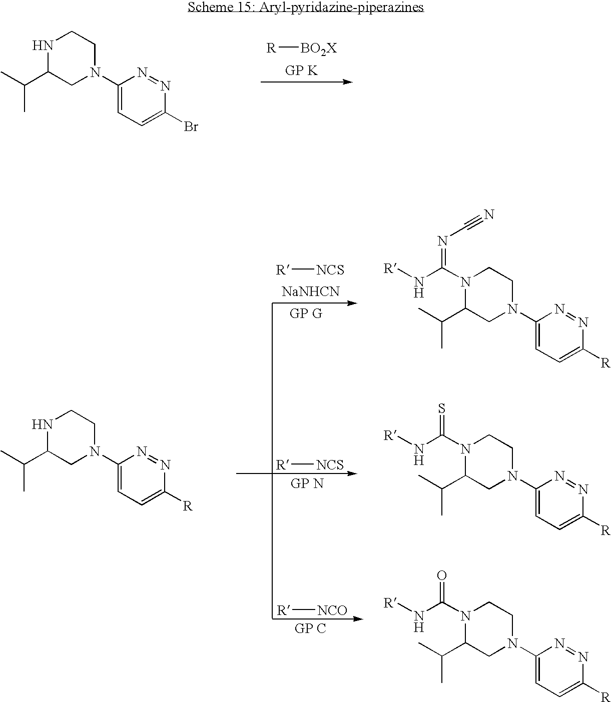

The compounds in table 15 were made according to general procedure K, followed by either general procedure G or C or N as illustrated in scheme 15. The specific procedure used for each compound is listed in table 15.

TABLE 15
Aryl-pyridazine-piperazines

| | | | | | Rt/min | |
|---|---|---|---|---|---|---|
| Ex # | R—BO₂X | Reagent | GP | Product | (method) | m/z |
| 15.1 | 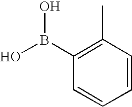 | 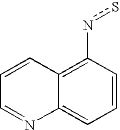 | N | 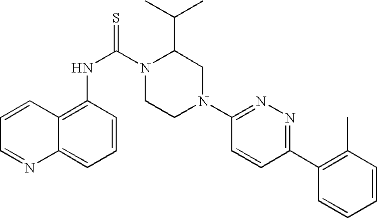 | 2.03 (d) | 481 (M − H)⁻ |
| 15.2 | 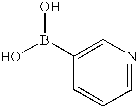 | 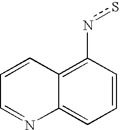 | N | 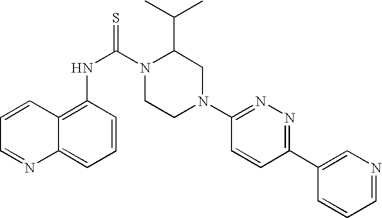 | 1.55 (d) | 468 (M − H)⁻ |
| 15.3 | 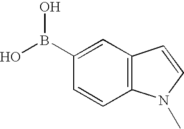 | 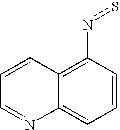 | N | 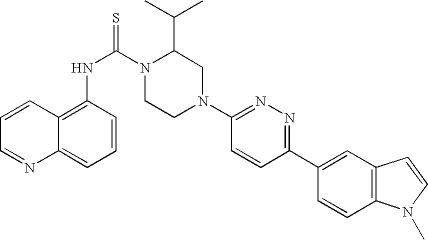 | 1.95 (d) | 520 (M − H)⁻ |
| 15.4 | 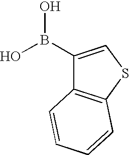 | 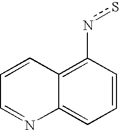 | N | 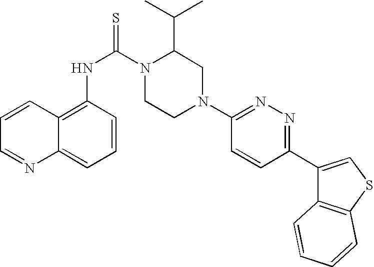 | 2.15 (d) | 523 (M − H)⁻ |
| 15.5 | 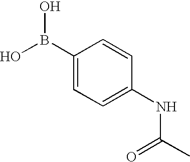 | 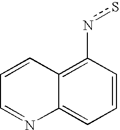 | N | 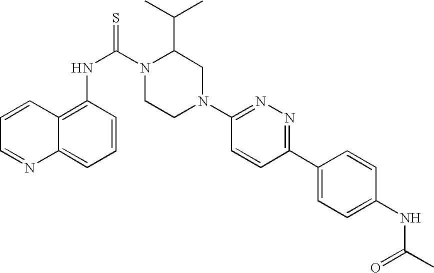 | 1.53 (d) | 524 (M − H)⁻ |
| 15.6 | 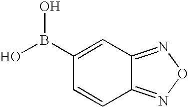 | 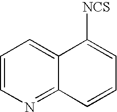 | N | 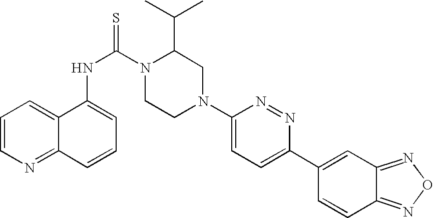 | 2.01 (d) | 509 (M − H)⁻ |
| 15.7 | 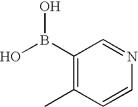 | 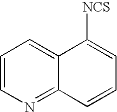 | N | 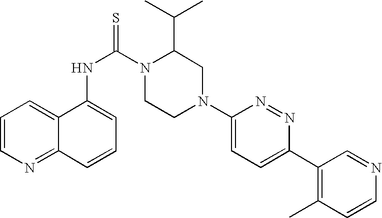 | 1.6 (d) | 482 (M − H)⁻ |
| 15.8 | 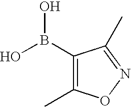 | 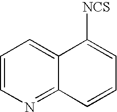 | N | 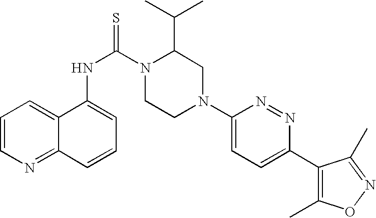 | 1.72 (d) | 488 (M + H)⁺ |
| 15.9 | 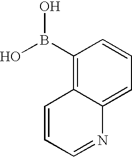 | 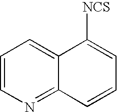 | N | 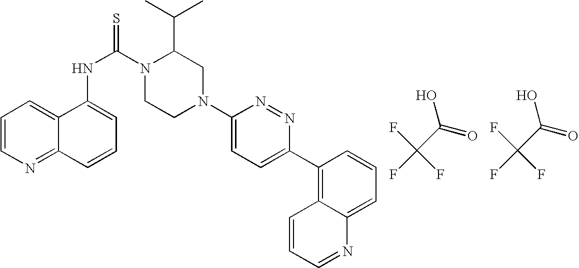 | 1.79 (d) | 518 (M − H)⁻ |
| 15.10 | 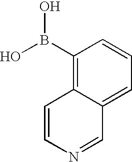 | 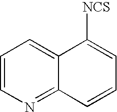 | N | 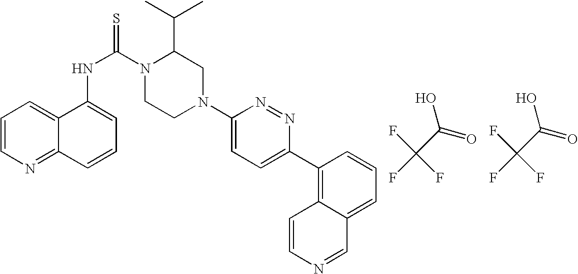 | 1.81 (d) | 518 (M − H)⁻ |
| 15.11 | 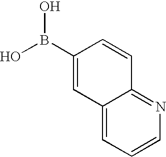 | 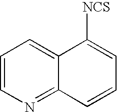 | N | 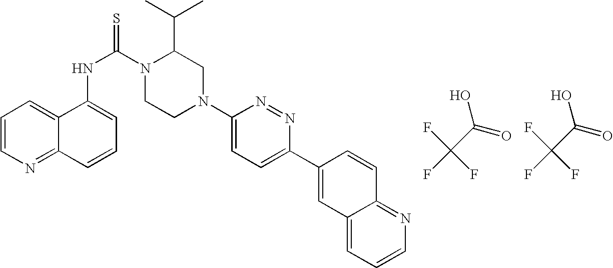 | 1.85 (d) | 518 (M − H)⁻ |
| 15.12 | 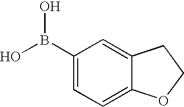 | 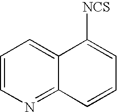 | N | 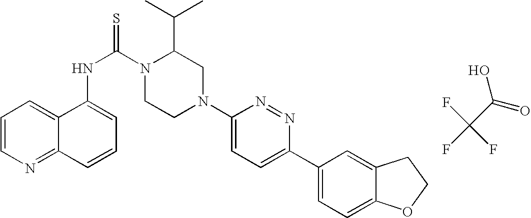 | 1.91 (d) | 509 (M − H)⁻ |
| 15.13 | 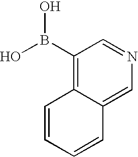 | 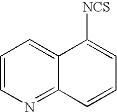 | N | 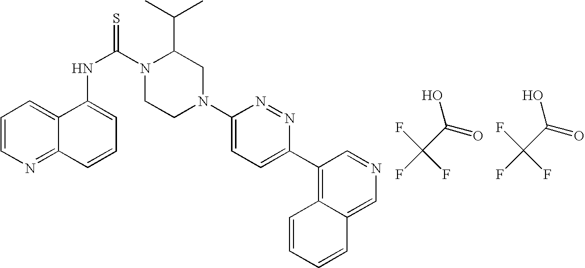 | 1.82 (d) | 518 (M − H)⁻ |
| 15.14 | 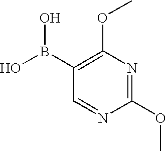 | 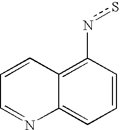 | N | 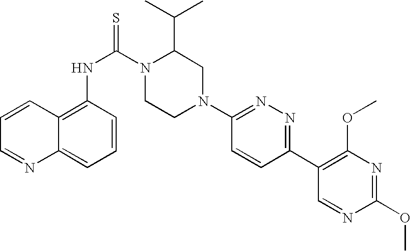 | 1.70 (d) | 529 (M − H)⁻ |
| 15.15 | 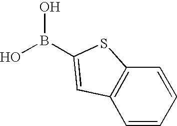 | 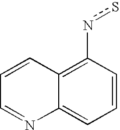 | N | 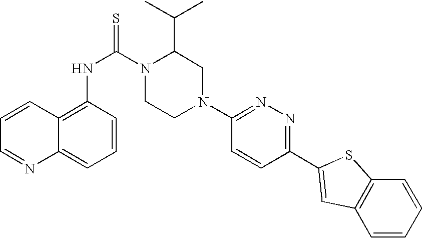 | 2.19 (d) | 523 (M − H)⁻ |
| 15.16 | 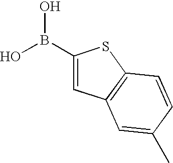 | 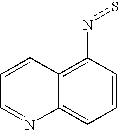 | N | 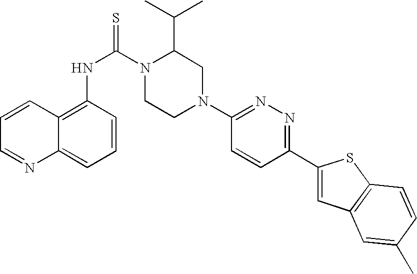 | 2.29 (d) | 537 (M − H)⁻ |
| 15.17 | 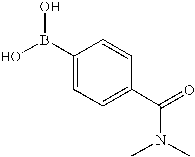 | 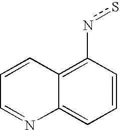 | N | 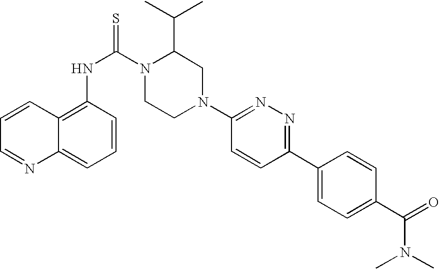 | 1.57 (d) | 538 (M − H)⁻ |
| 15.18 | 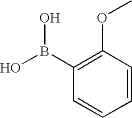 | 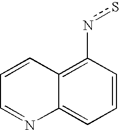 | N | 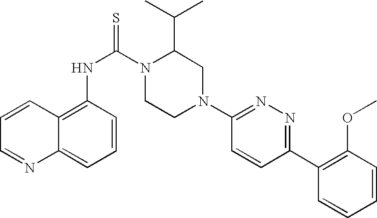 | 1.88 (d) | 497 (M − H)⁻ |
| 15.19 | 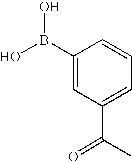 | 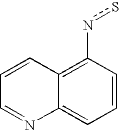 | N | 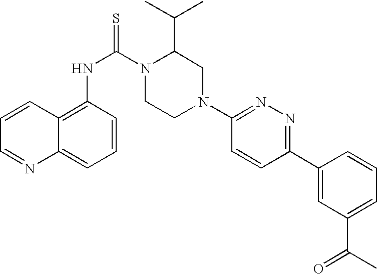 | 1.80 (d) | 509 (M − H)⁻ |
| 15.20 | 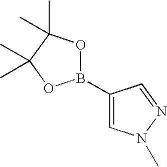 | 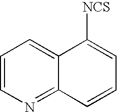 | N | 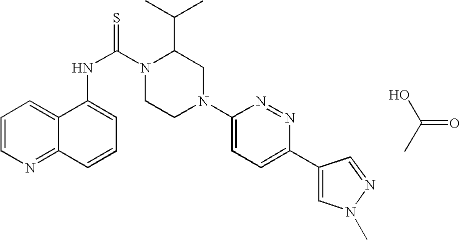 | 1.31 (d) | 471 (M − H)⁻ |
| 15.21 | 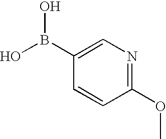 | 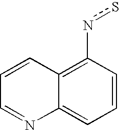 | N | 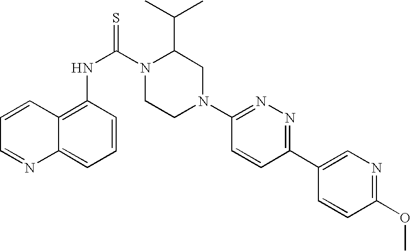 | 1.74 (d) | 498 (M − H)⁻ |
| 15.22 | 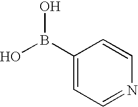 | 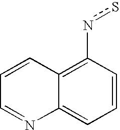 | N | 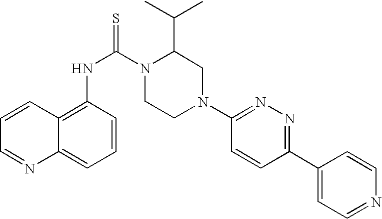 | 1.56 (d) | 468 (M − H)⁻ |
| 15.23 | 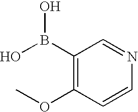 | 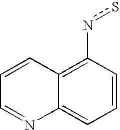 | N | 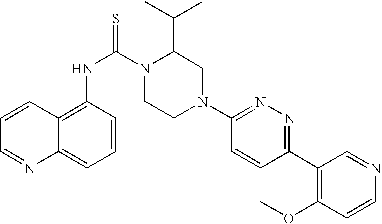 | 1.53 (d) | 498 (M − H)⁻ |
| 15.24 | 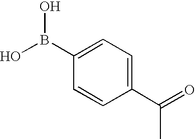 | 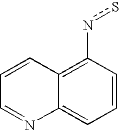 | N | 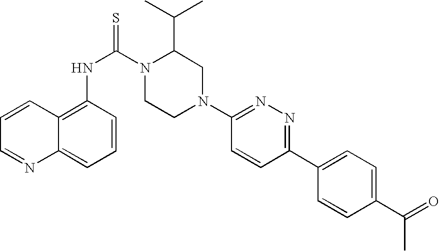 | 1.80 (d) | 509 (M − H)⁻ |
| 15.25 | 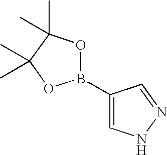 | 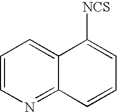 | N | 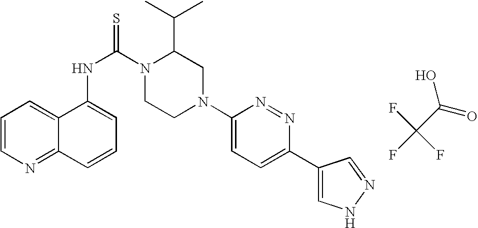 | 1.29 (d) | 459 (M + H)⁺ |
| 15.26 | 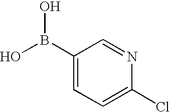 | 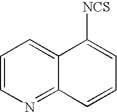 | N | 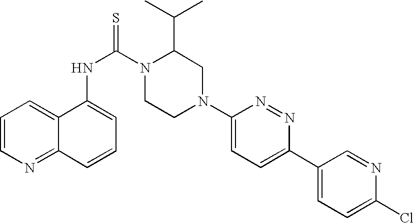 | 1.8 (d) | 502 (M − H)⁻ |
| 15.27 | 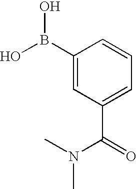 | 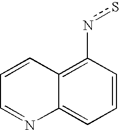 | N | 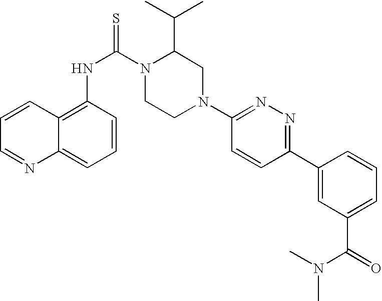 | 1.54 (d) | 538 (M − H)⁻ |
| 15.28 | 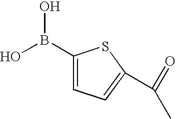 | 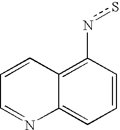 | N | 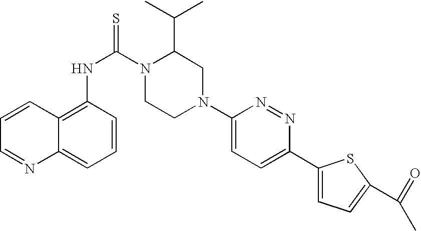 | 1.79 (d) | 515 (M − H)⁻ |
| 15.29 | 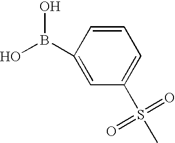 | 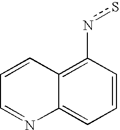 | N | 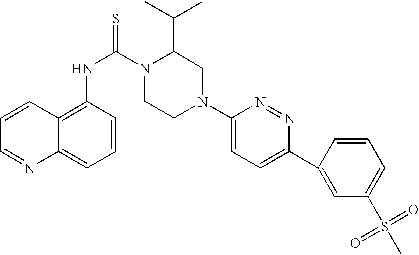 | 1.67 (d) | 545 (M − H)⁻ |
| 15.30 | 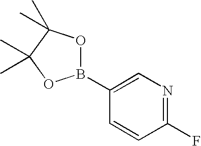 | 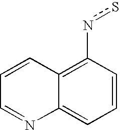 | N | 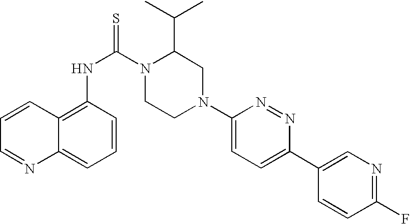 | 1.73 (d) | 486 (M − H)⁻ |
| 15.31 | 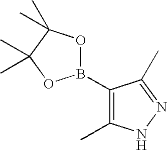 | 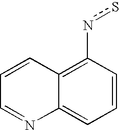 | N | 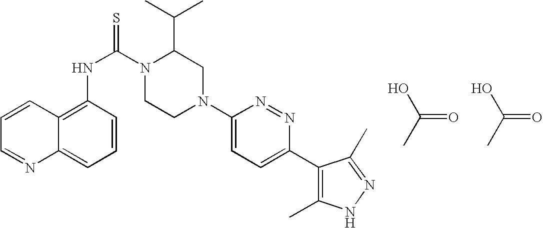 | 1.39 (d) | 485 (M − H)⁻ |
| 15.32 | 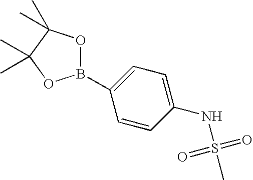 | 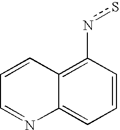 | N | 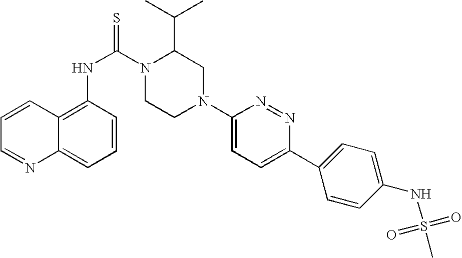 | 1.59 (d) | 560 (M − H)⁻ |
| 15.33 | 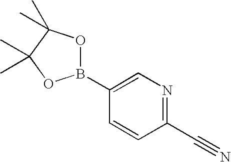 | 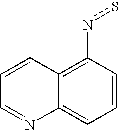 | N | 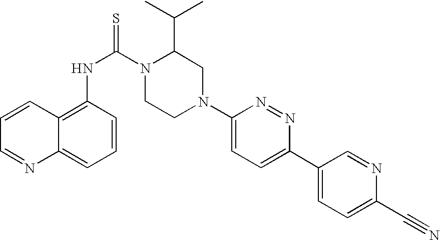 | 1.75 (d) | 493 (M − H)⁻ |
| 15.34 | 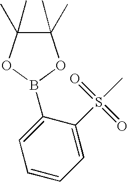 | 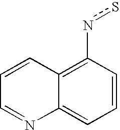 | N | 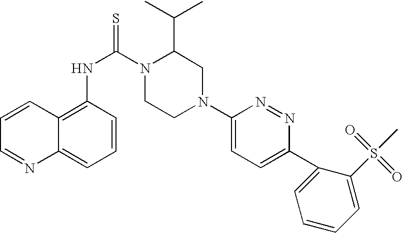 | 1.62 (d) | 545 (M − H)⁻ |
| 15.35 | 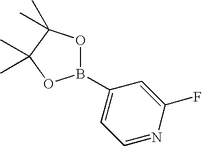 | 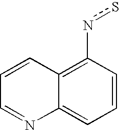 | N | 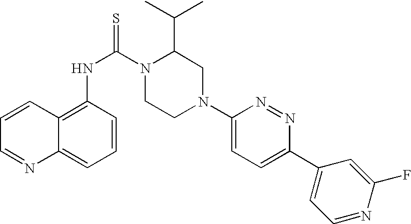 | 1.75 (d) | 486 (M − H)⁻ |
| 15.36 | 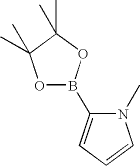 | 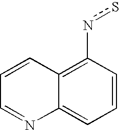 | N | 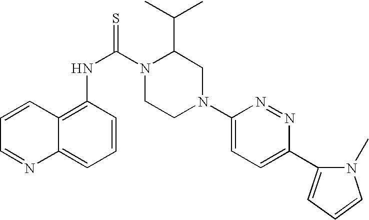 | 1.85 (d) | 470 (M − H)⁻ |
| 15.37 | 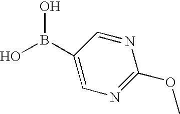 | 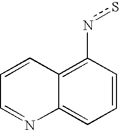 | N | 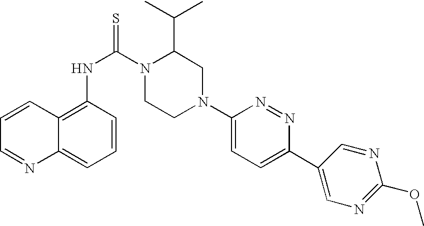 | 1.54 (d) | 499 (M − H)⁻ |
| 15.38 | 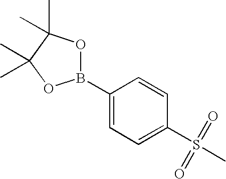 | 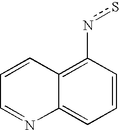 | N | 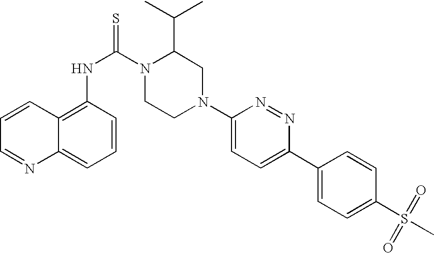 | 1.62 (d) | 545 (M − H)⁻ |
| 15.39 | 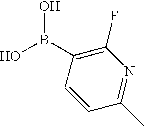 | 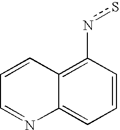 | N | 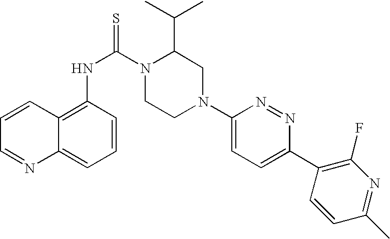 | 1.87 (d) | 500 (M − H)⁻ |
| 15.40 | 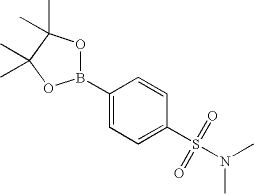 | 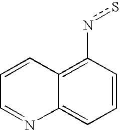 | N | 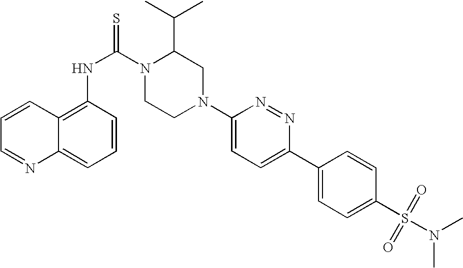 | 1.89 (d) | 574 (M − H)⁻ |
| 15.41 | 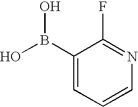 | 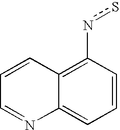 | N | 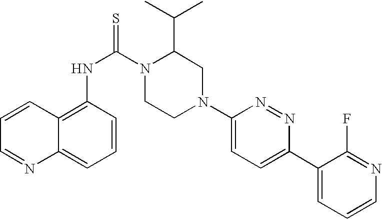 | 1.70 (d) | 486 (M − H)⁻ |
| 15.42 | 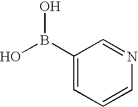 | 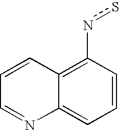 | G | 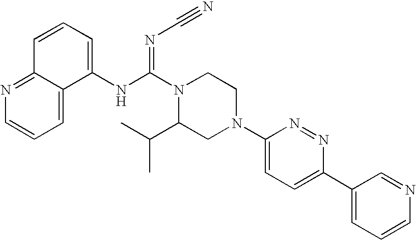 | 1.43 (d) | 476 (M − H)⁻ |
| 15.43 | 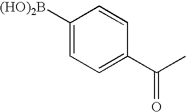 | 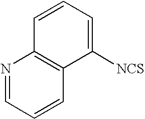 | G | 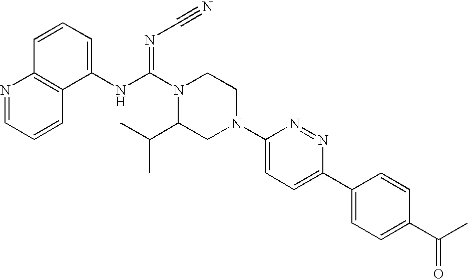 | 1.77 (b) | 519 (M + H)⁺ |
| 15.44 | 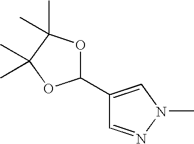 | 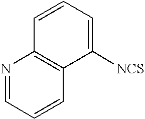 | G | 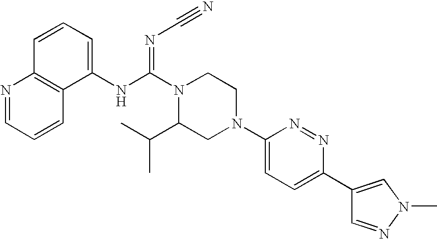 | 1.50 (b) | 481 (M + H)⁺ |
| 15.45 | 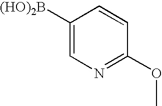 | 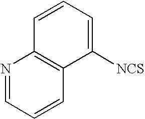 | G | 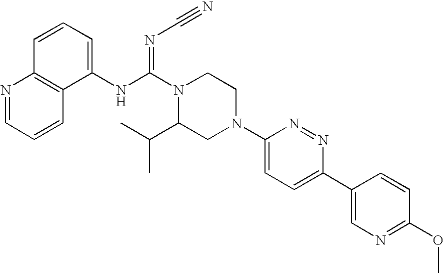 | 1.73 (b) | 508 (M + H)⁺ |
| 15.46 | 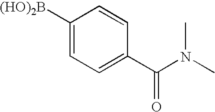 | 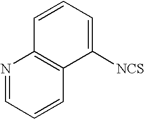 | G | 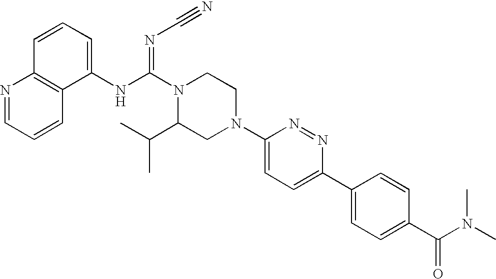 | 1.58 (b) | 548 (M + H)⁺ |
| 15.47 | 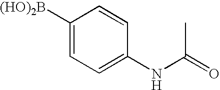 | 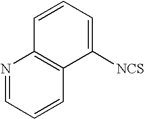 | G | 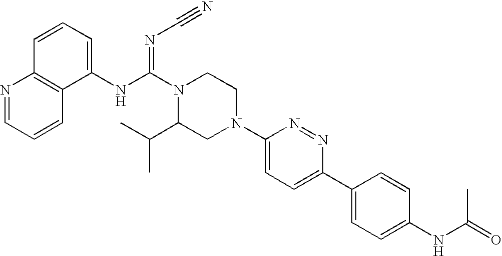 | 1.57 (b) | 534 (M − H)⁺ |
| 15.48 | 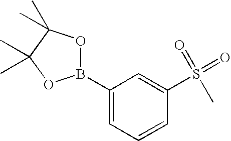 | 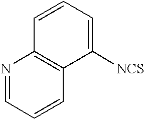 | G | 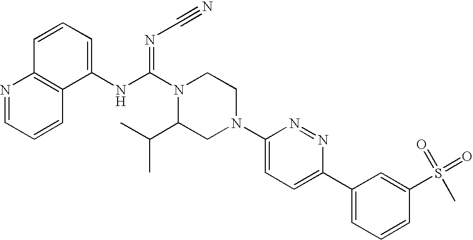 | 1.68 (b) | 555 (M + H)⁺ |
| 15.49 | 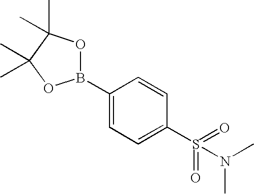 | 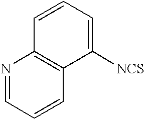 | G | 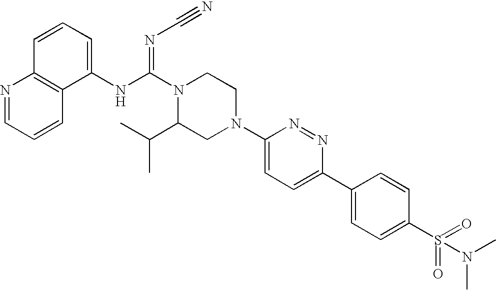 | 1.81 (b) | 584 (M + H)⁺ |
| 15.50 | 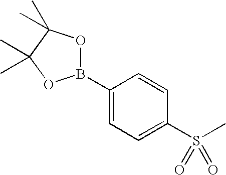 | 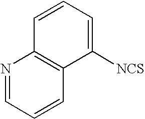 | G | 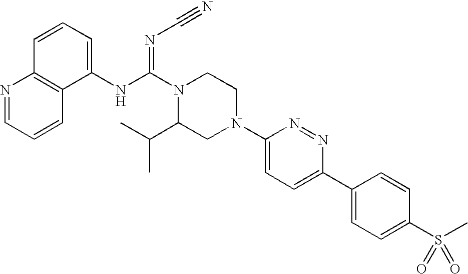 | 1.63 (b) | 555 (M + H)⁺ |
| 15.51 | 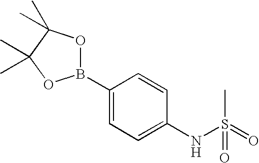 | 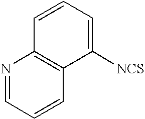 | G | 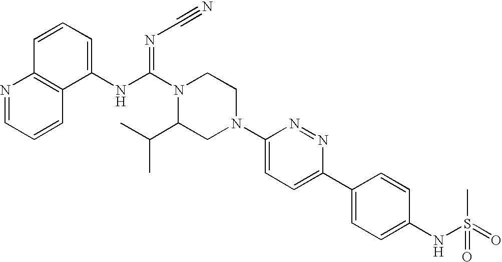 | 1.62 (b) | 570 (M + H)⁺ |
| 15.52 | 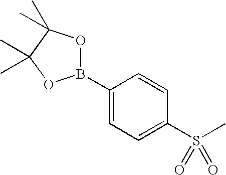 | 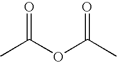 | C | 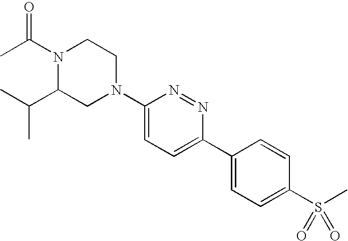 | 1.66 (b) | 403 (M + H)⁺ |
| 15.53 | 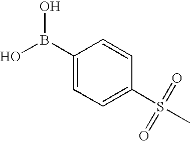 | 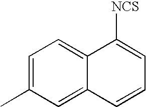 | G | 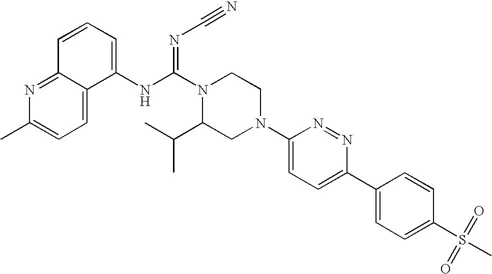 | 2.14 (a) | 567 (M − H)⁻ |

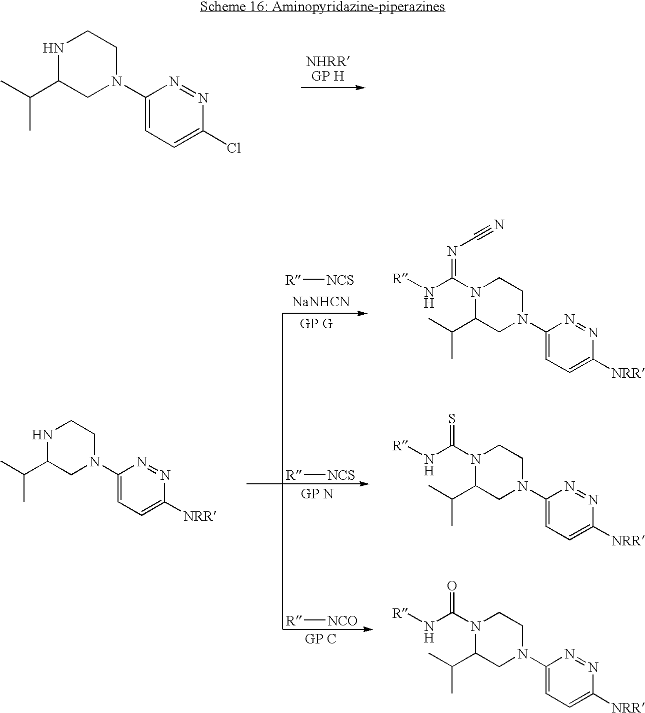

The compounds in table 16 were made according to general procedure H, followed by either general procedure G or C or N as illustrated in scheme 16. The specific procedure used for each compound is listed in table 16.

TABLE 16
Aminopyridazine-piperazines

| | | | | | Rt/min | |
|---|---|---|---|---|---|---|
| | | | | | (meth- | |
| Ex # | NHRR′ | Reagent | GP | Product | od) | m/z |
| 16.1 | 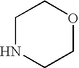 | 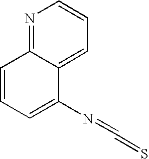 | G | 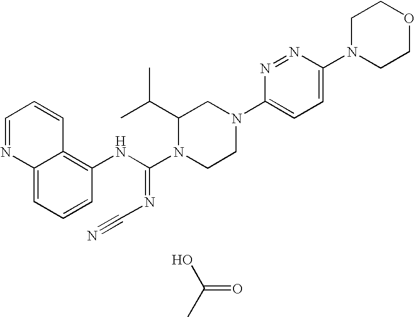 | 1.98 (d) | 486 (M +H)⁺ |
| 16.2 | 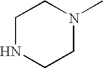 | 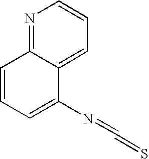 | N | 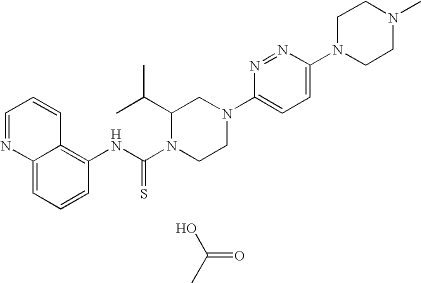 | 1.44 (d) | 491 (M +H)⁺ |
| 16.3 | 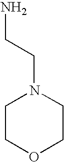 | 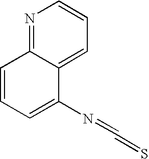 | G | 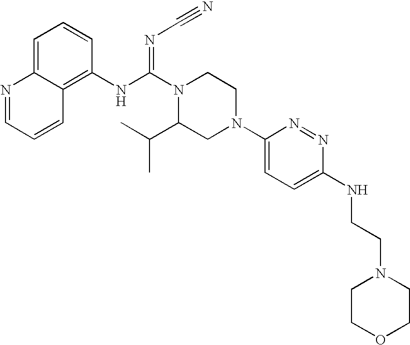 | 1.39 (d) | 529 (M +H)⁺ |
| 16.4 | 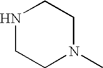 | 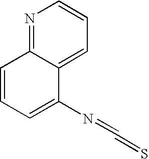 | G | 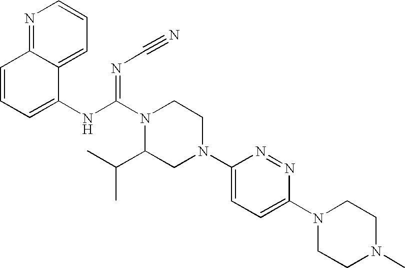 | 1.43 (d) | 499 (M +H)⁺ |
| 16.5 | 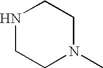 | 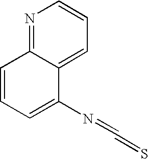 | C | 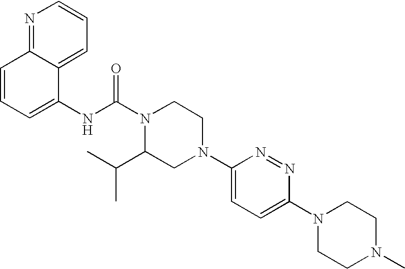 | 1.42 | 475 (M +H)⁺ |
| 16.6 | 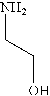 | 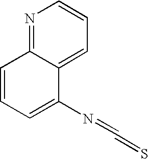 | N | 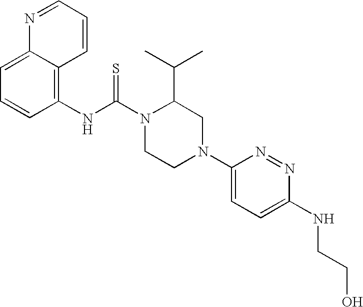 | 1.42 | 450 (M −H)⁻ |
| 16.7 | 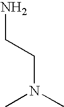 | 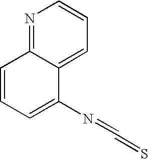 | N | 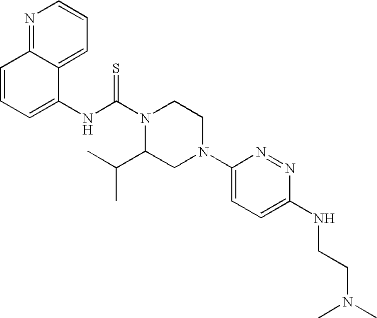 | 1.18 (d) | 479 (M +H)⁺ |
| 16.8 | 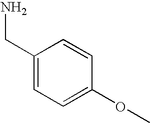 | 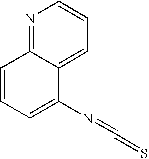 | N | 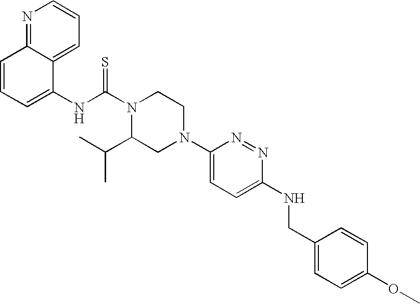 | 1.58 (d) | 528 (M +H)⁺ |
| 16.9 | NH₃ | 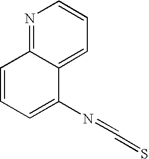 | N | 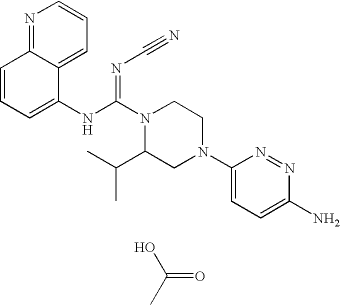 | 1.42 (d) | 416 (M +H)⁺ |

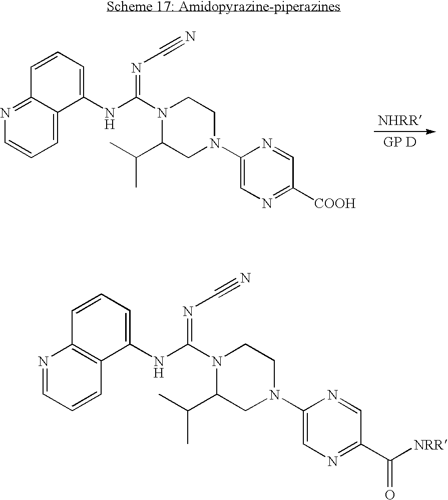

The compounds in table 17 were made according to general procedure D, as illustrated in scheme 17.

TABLE 17
Amidopyrazine-piperazines

| | | | Rt/min | |
|---|---|---|---|---|
| Ex. | NHRR′ | Product | (method) | m/z |
| 17.1 | 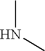 | 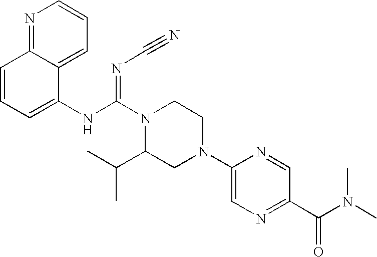 | 142 (d) | 470 (M − H)⁻ |
| 17.2 | 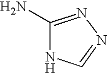 | 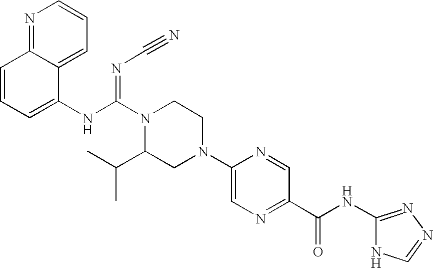 | 1.37 (d) | 509 (M − H)⁻ |
| 17.3 | 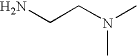 | 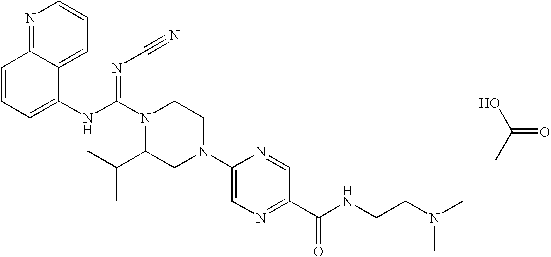 | 1.21 (d) | 515 (M + H)⁺ |
| 17.4 | 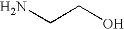 | 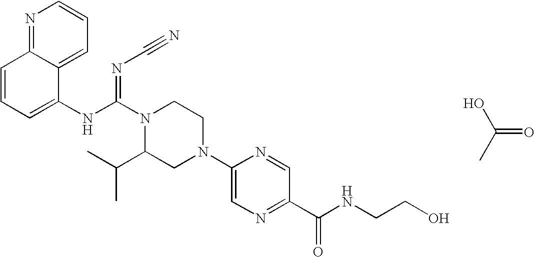 | 1.31 (d) | 487 (M − H)⁻ |
| 17.5 | 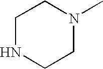 | 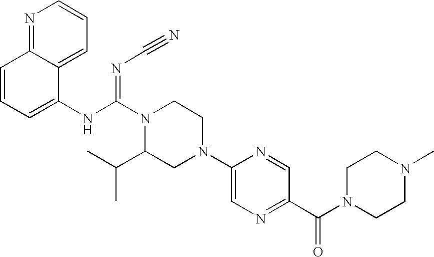 | 1.20 (d) | 526 (M − H)⁻ |
| 17.6 | 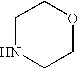 | 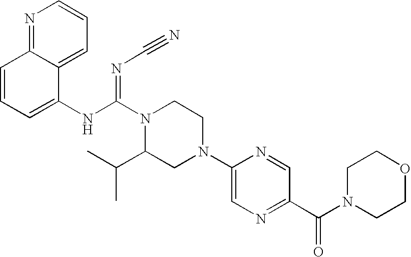 | 1.39 (d) | 512 (M − H)⁻ |
| 17.7 | 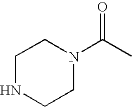 | 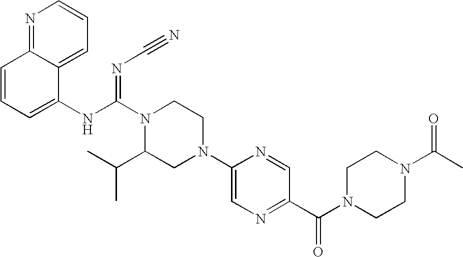 | 1.31 (d) | 555 (M + H)⁺ |
| 17.8 | 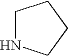 | 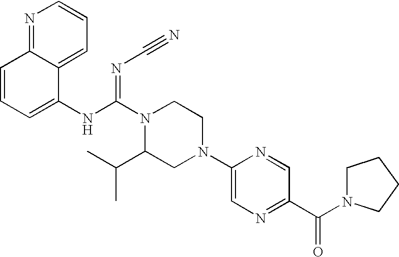 | 1.50 (d) | 497 (M − H)⁻ |
| 17.9 | 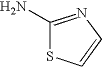 | 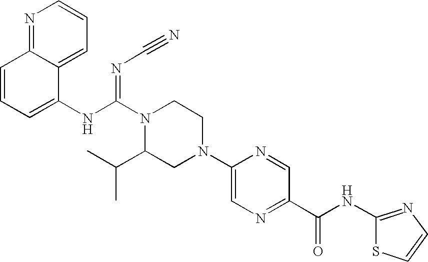 | 1.61 (d) | 527 (M + H)⁺ |
| 17.10 | 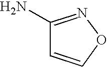 | 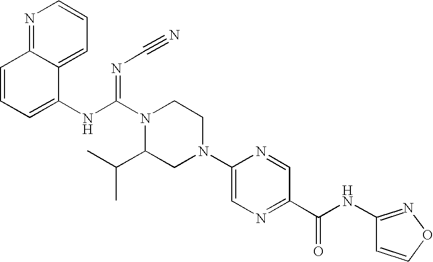 | 1.57 (d) | 509 (M − H)⁻ |
| 17.11 | 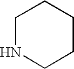 | 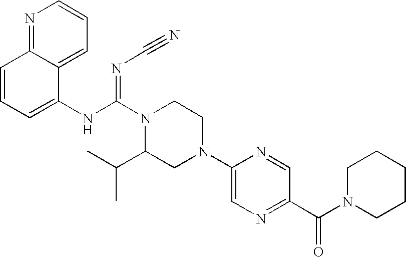 | 1.55 (d) | 510 (M − H)⁻ |
| 17.12 | 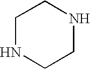 | 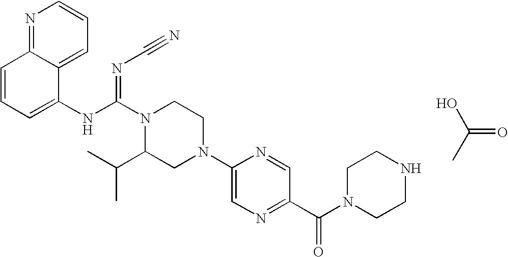 | 1.18 (d) | 512 (M − H)⁻ |
| 17.13 | 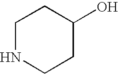 | 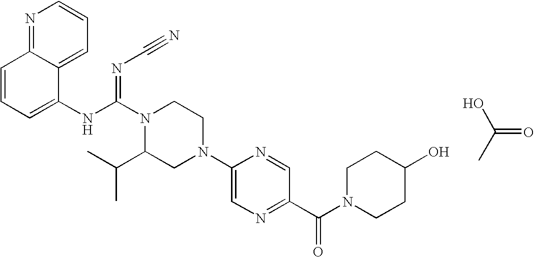 | 1.29 (d) | 526 (M − H)⁻ |
| 17.14 | 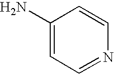 | 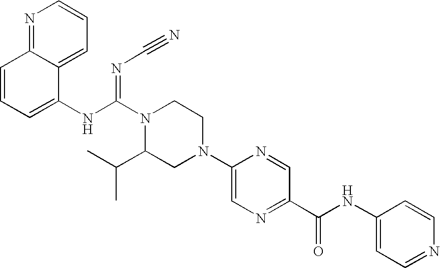 | 1.53 (d) | 521 (M + H)⁺ |
| 17.15 | 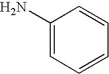 | 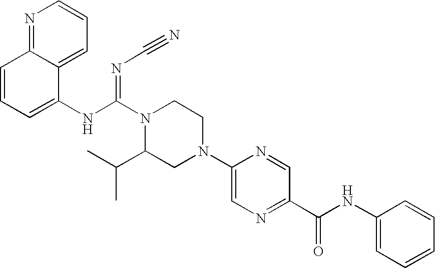 | 1.76 (d) | 518 (M − H)⁻ |
| 17.16 | NH₃ | 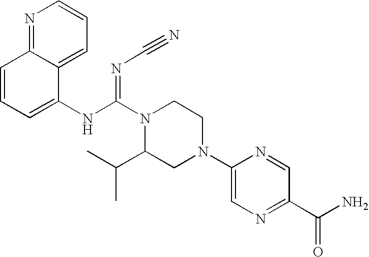 | 1.76 (d) | 444 (M + H)⁺ |

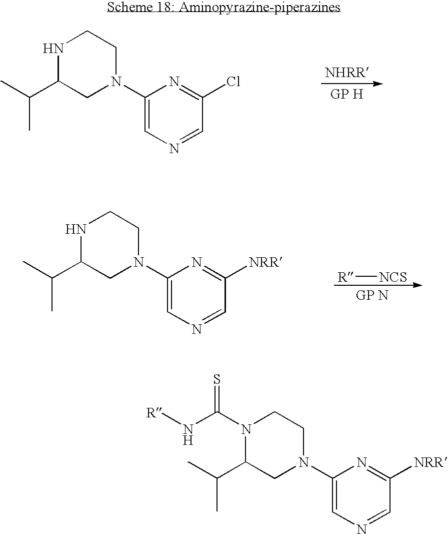

The compounds in table 18 were made according to general procedure H, followed by general procedure N, as illustrated in scheme 18.

TABLE 18
Aminopyrazine-piperazines

| Ex # | NHRR′ | R″—NCS | MOLSTRUCTURE |
|---|---|---|---|
| 18.1 | 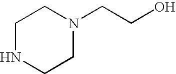 | 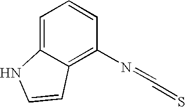 | 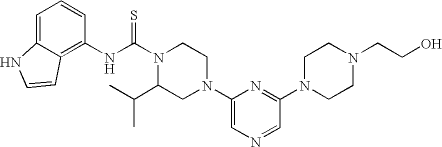 |
| 18.2 | 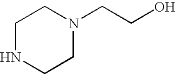 | 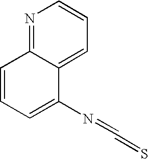 | 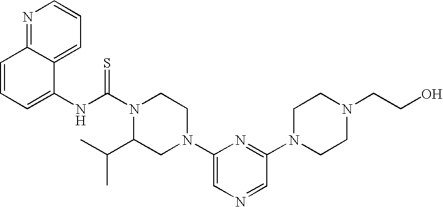 |
| 18.3 | 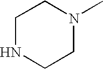 | 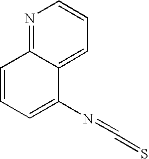 | 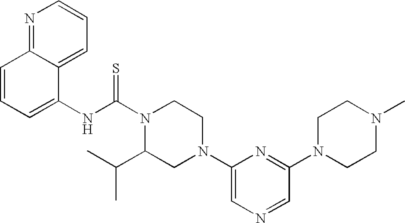 |
| 18.4 | 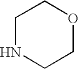 | 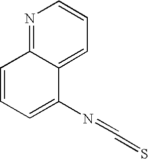 | 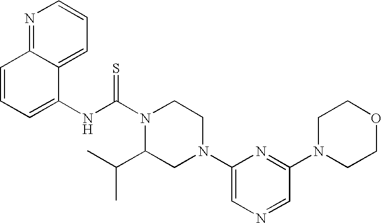 |
| 18.5 | 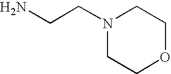 | 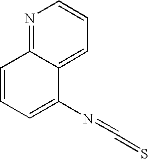 | 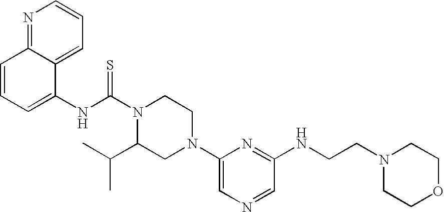 |
| 18.6 | 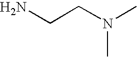 | 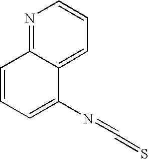 | 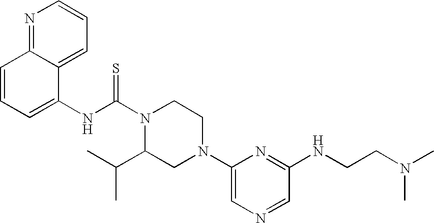 |
| 18.7 | 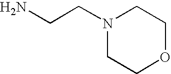 | 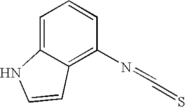 | 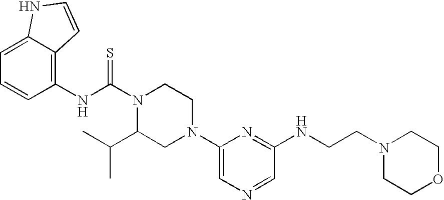 |
| 18.8 | 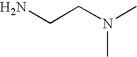 | 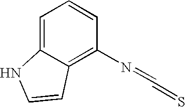 | 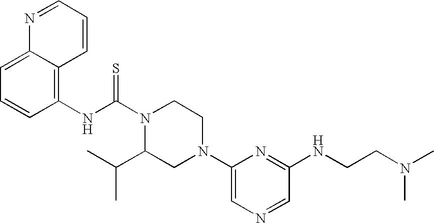 |
| 18.9 | 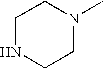 | 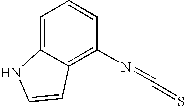 | 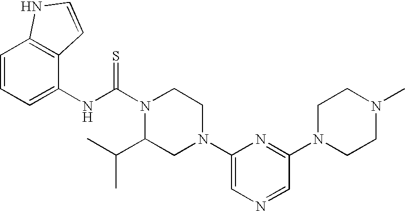 |
| 18.10 | 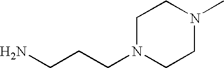 | 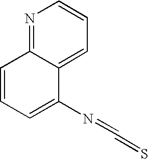 | 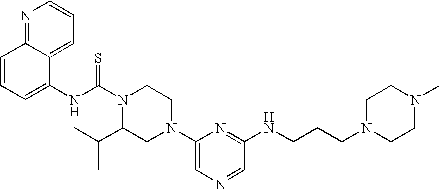 |

| | Rt/min | |
|---|---|---|
| Ex # | (method) | m/z |
| 18.1 | 1.59 (d) | 509 (M + H)⁺ |
| 18.2 | 1.36 (d) | 521 (M + H)⁺ |
| 18.3 | 1.37 (d) | 490 (M − H)⁻ |
| 18.4 | 1.37 (d) | 479 (M + H)⁺ |
| 18.5 | 1.40 (d) | 519 (M − H)⁻ |
| 18.6 | 1.37 (d) | 477 (M − H)⁻ |
| 18.7 | 1.48 (d) | 509 (M + H)⁺ |
| 18.8 | 1.43 (d) | 467 (M + H)⁺ |
| 18.9 | 1.46 (d) | 477 (M − H)⁻ |
| 18.10 | 1.38 (d) | 546 (M − H)⁻ |

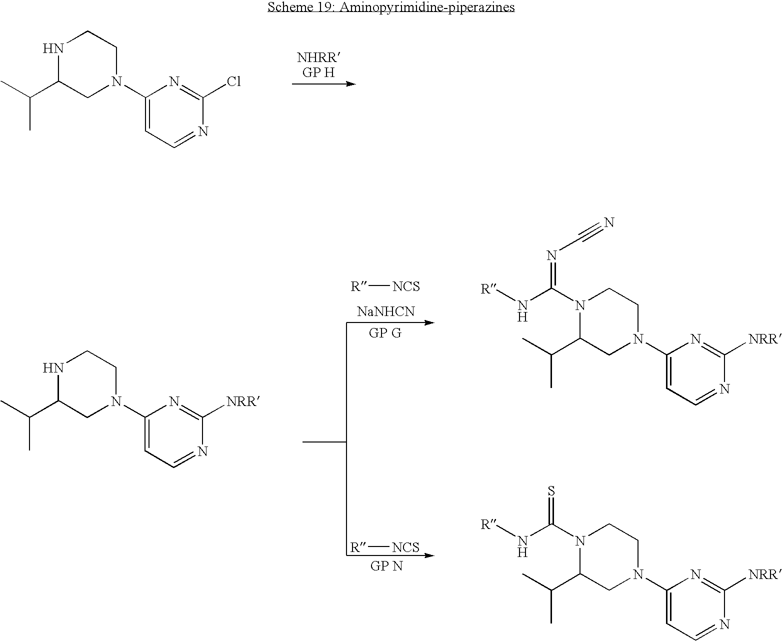

The compounds in table 19 were made according to general procedure H, followed by either general procedure G or N, as illustrated in scheme 19. The specific procedure used for each compound is listed in table 19.

TABLE 19
Aminopyrimidine-piperazines

| Ex # | NHRR′ | R″—NCS | GP | Product |
|---|---|---|---|---|
| 19.1 | 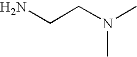 | 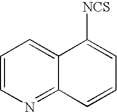 | G | 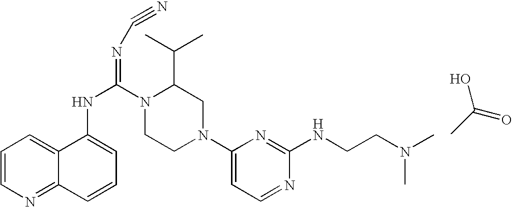 |
| 19.2 | 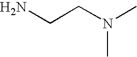 | 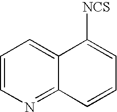 | G | 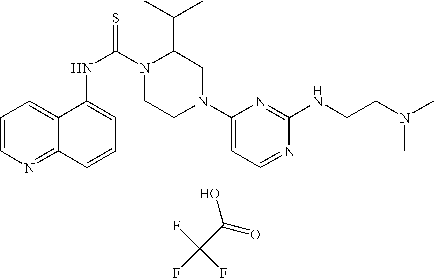 |
| 19.3 | 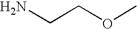 | 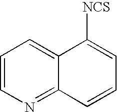 | G | 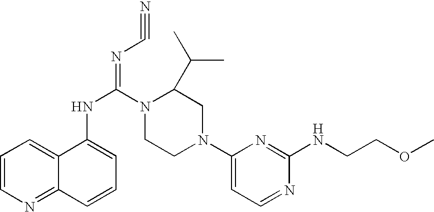 |
| 19.4 | 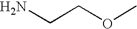 | 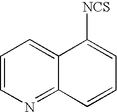 | G | 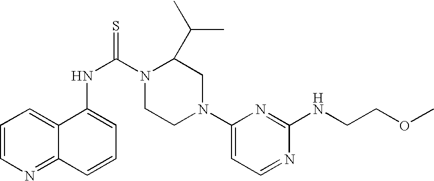 |
| 19.5 | 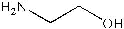 | 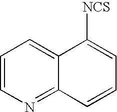 | G | 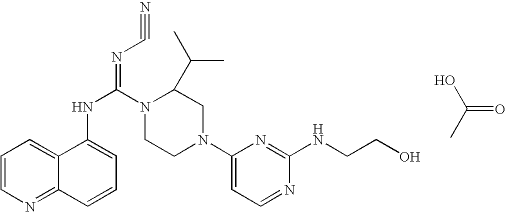 |
| 19.6 | 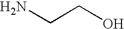 | 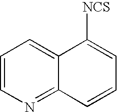 | G | 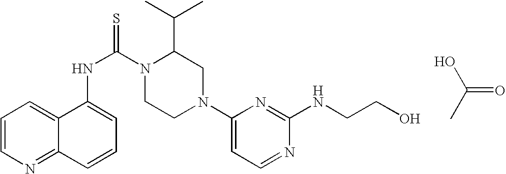 |
| 19.7 | 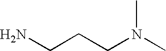 | 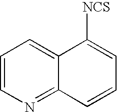 | G | 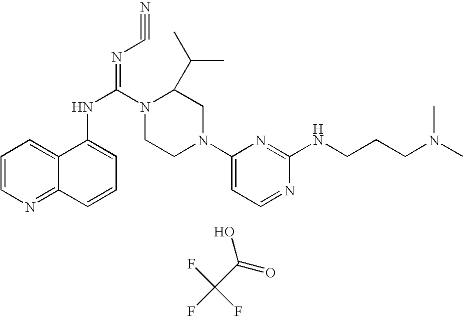 |
| 19.8 | 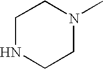 | 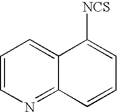 | G | 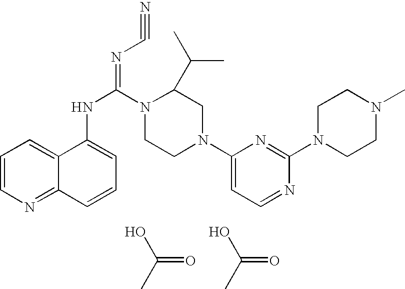 |
| 19.9 | 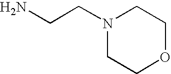 | 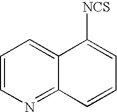 | G | 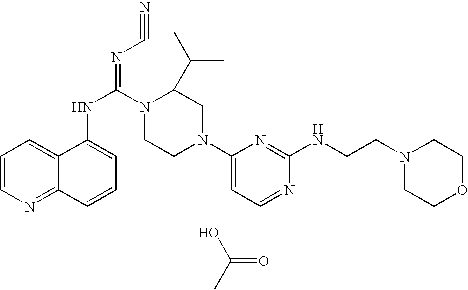 |
| 19.10 | 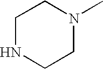 | 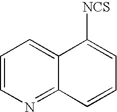 | G | 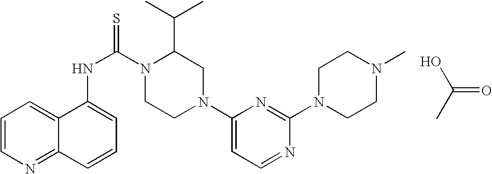 |
| 19.11 | 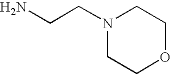 | 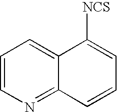 | G | 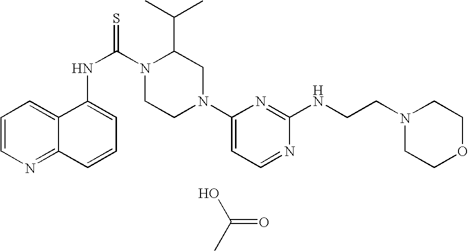 |
| 19.12 | 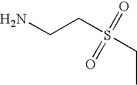 | 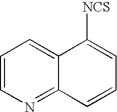 | N | 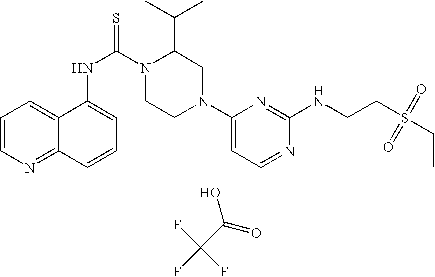 |
| 19.13 | 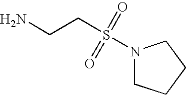 | 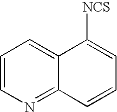 | N | 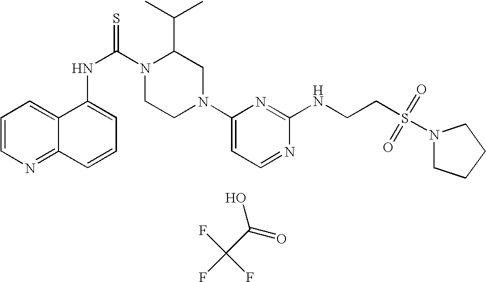 |

| Ex # | Rt/min (method) | m/z |
|---|---|---|
| 19.1 | 1.32 (d) | 487 (M + H)⁺ |
| 19.2 | 1.66 (d) | 479 (M + H)⁺ |
| 19.3 | 1.43 (d) | 472 (M − H)⁻ |
| 19.4 | 1.65 (d) | 464 (M − H)⁻ |
| 19.5 | 1.08 (d) | 460 (M + H)⁺ |
| 19.6 | 1.34 (d) | 450 (M − H)⁻ |
| 19.7 | 1.46 (d) | 499 (M − H)⁻ |
| 19.8 | 1.58 (d) | 497 (M − H)⁻ |
| 19.9 | 1.4 (d) | 527 (M − H)⁻ |
| 19.10 | 1.93 (d) | 489 (M − H)⁻ |
| 19.11 | 1.65 (d) | 519 (M − H)⁻ |
| 19.12 | 1.35 (d) | 526 (M − H)⁻ |
| 19.13 | 1.64 (d) | 567 (M − H)⁻ |

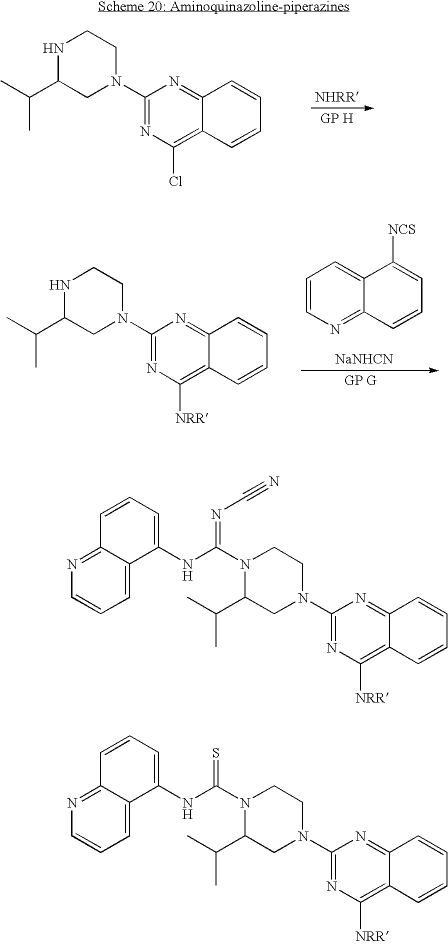

The compounds in table 20 were made according to general procedure H, followed by general procedure G, as illustrated in scheme 20.

TABLE 20
Aminoquinazoline-piperazines

| | | | Rt/min | |
|---|---|---|---|---|
| Ex # | NHRR′ | Product | (method) | m/z |
| 20.1 | 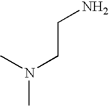 | 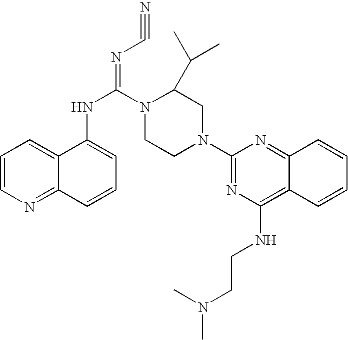 | 1.38 (d) | 537 (M + H)⁺ |
| 20.2 | 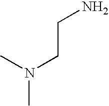 | 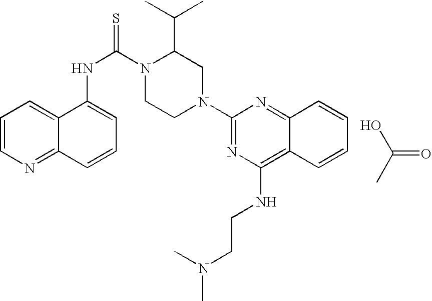 | 1.59 (d) | 529 (M + H)⁺ |
| 20.3 | 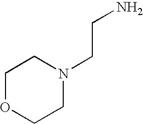 | 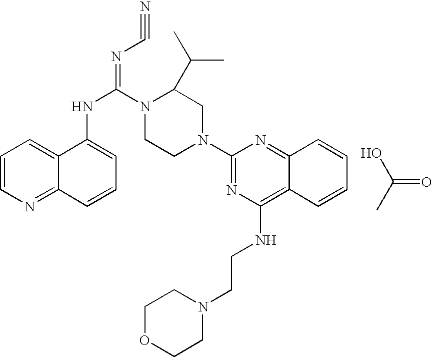 | 1.46 (d) | 579 (M + H)⁺ |
| 20.4 | NH₃ | 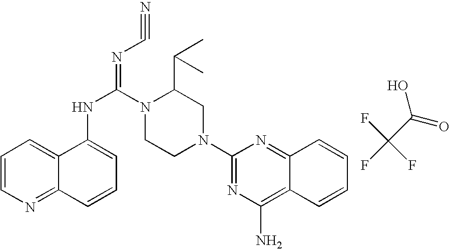 | 1.55 (d) | 466 (M + H)⁺ |

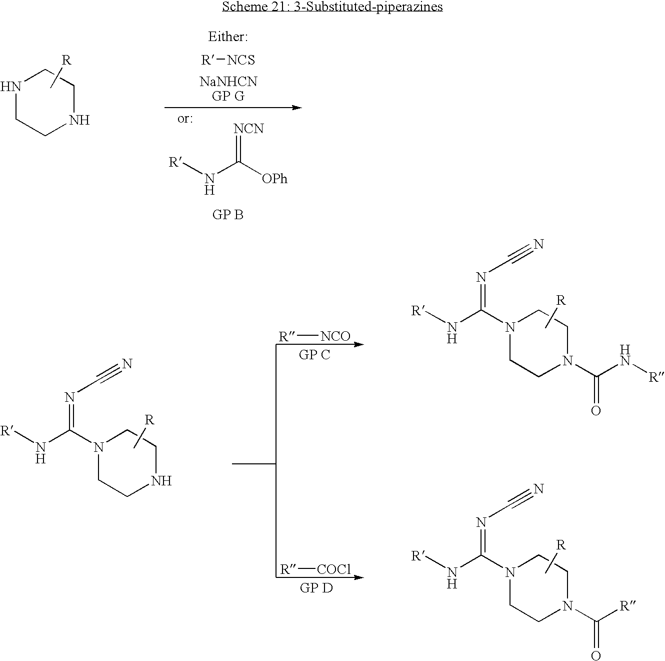

The compounds in table 21 were made according to general procedure B or G, followed by either general procedure C or D, as illustrated in scheme 21. The specific procedure used for each compound is listed in table 21.

TABLE 21
3-Substituted-piperazines

| Ex # | Piperazine | R′—NCS or 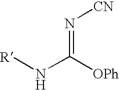 | GP | R″—NCO or R″—COCl | GP |
|---|---|---|---|---|---|
| 21.1 | 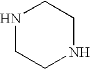 | 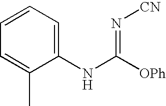 | B | 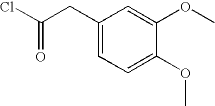 | D |
| 21.2 | 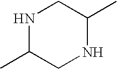 | 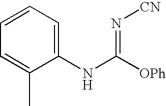 | B | 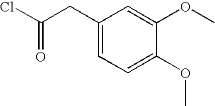 | D |
| 21.3 | 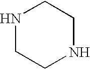 | 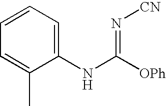 | B | 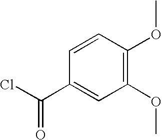 | D |
| 21.4 | 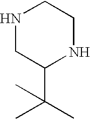 | 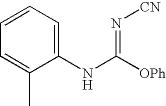 | B | 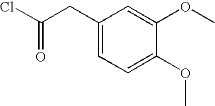 | D |
| 21.5 | 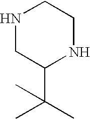 | 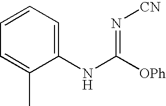 | B | 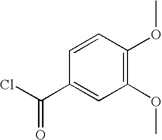 | D |
| 21.6 | 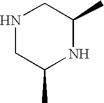 | 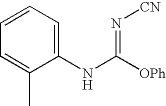 | B | 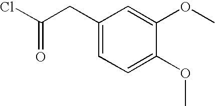 | D |
| 21.7 | 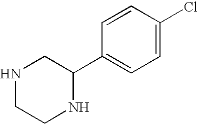 | 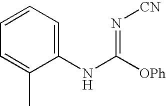 | B | 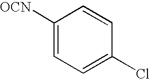 | C |
| 21.8 | 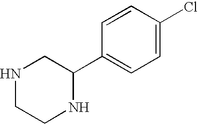 | 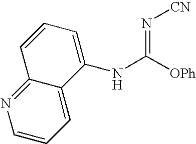 | B | 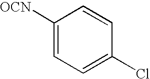 | C |
| 21.9 | 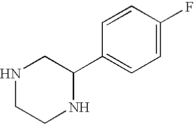 | 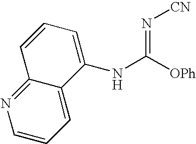 | B | 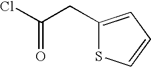 | D |
| 21.10 | 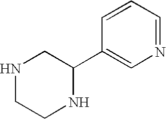 | 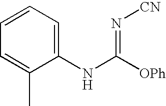 | B | 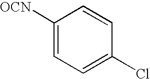 | C |
| 21.11 | 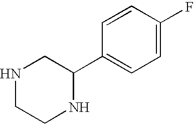 | 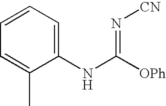 | B | 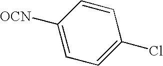 | C |
| 21.12 | 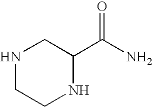 | 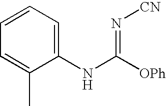 | B | 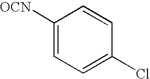 | C |
| 21.13 | 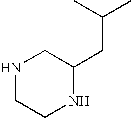 | 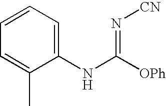 | B | 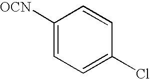 | C |
| 21.14 | 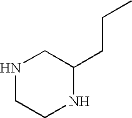 | 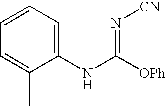 | B | 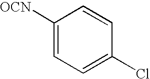 | C |
| 21.15 | 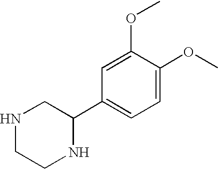 | 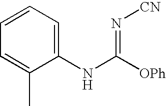 | B | 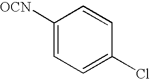 | C |
| 21.16 | 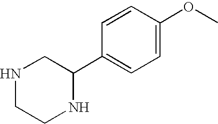 | 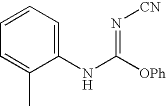 | B | 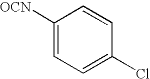 | C |
| 21.17 | 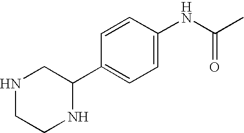 | 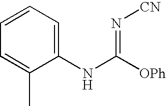 | B | 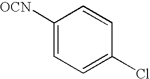 | C |
| 21.18 | 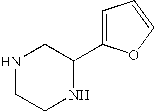 | 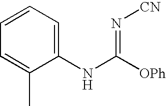 | B | 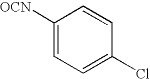 | C |
| 21.19 | 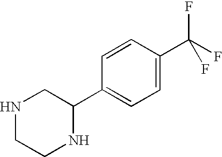 | 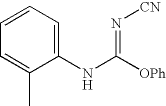 | B | 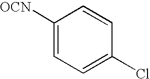 | C |
| 21.20 | 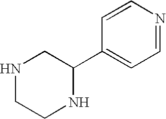 | 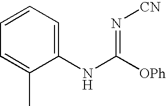 | B | 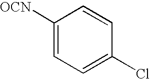 | C |
| 21.21 | 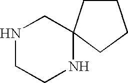 | 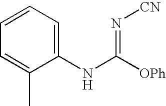 | B | 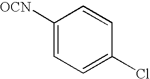 | C |
| 21.22 | 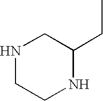 | 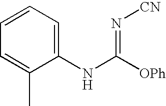 | B | 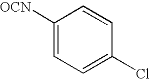 | C |
| 21.23 | 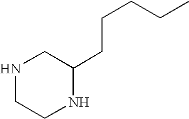 | 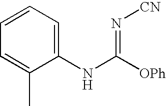 | B | 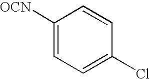 | C |
| 21.24 | 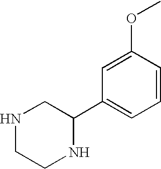 | 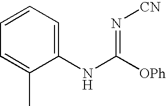 | B | 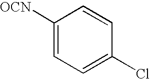 | C |
| 21.25 | 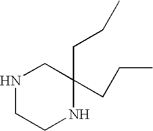 | 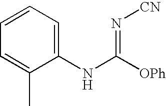 | B | 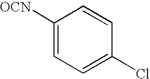 | C |
| 21.26 | 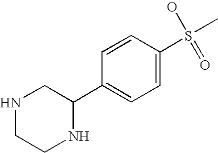 | 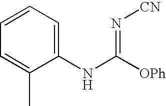 | B | 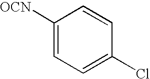 | C |
| 21.27 | 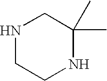 | 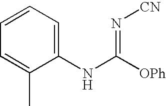 | B | 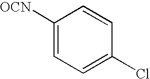 | C |
| 21.28 | 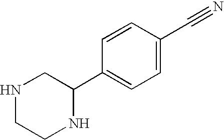 | 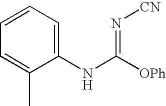 | B | 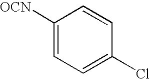 | C |
| 21.29 | 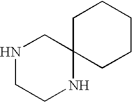 | 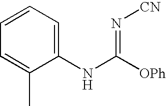 | B | 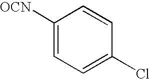 | C |
| 21.30 | 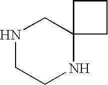 | 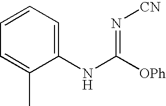 | B | 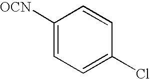 | C |
| 21.31 | 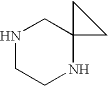 | 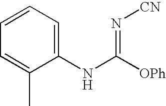 | B | 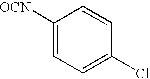 | C |
| 21.32 | 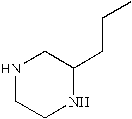 | 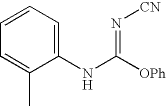 | B | 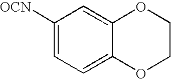 | C |
| 21.33 | 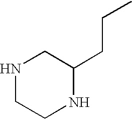 | 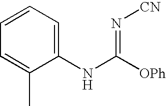 | B | 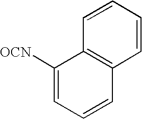 | C |
| 21.34 | 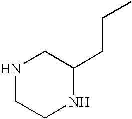 | 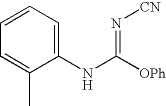 | B | 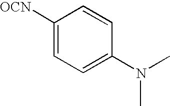 | C |
| 21.35 | 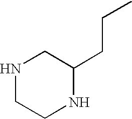 | 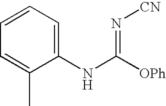 | B | 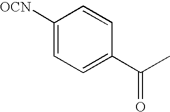 | C |
| 21.36 | 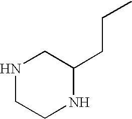 | 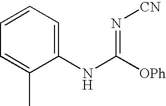 | B | 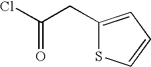 | D |
| 21.37 | 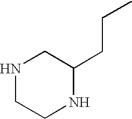 | 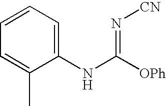 | B | 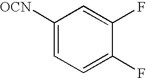 | C |
| 21.38 | 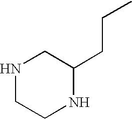 | 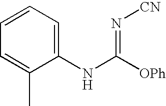 | B | 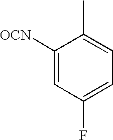 | C |
| 21.39 | 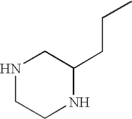 | 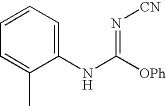 | B | 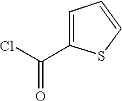 | D |
| 21.40 | 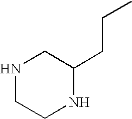 | 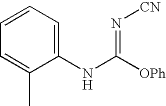 | B | 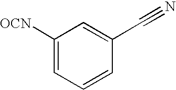 | C |
| 21.41 | 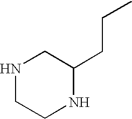 | 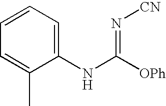 | B | 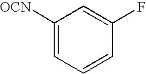 | C |
| 21.42 | 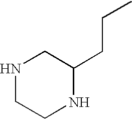 | 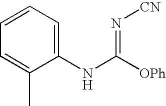 | B | 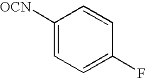 | C |
| 21.43 | 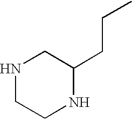 | 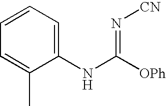 | B | 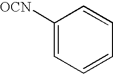 | C |
| 21.44 | 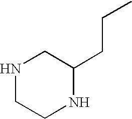 | 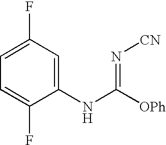 | B | 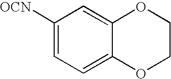 | C |
| 21.45 | 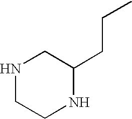 | 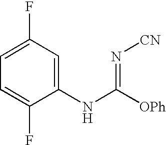 | B | 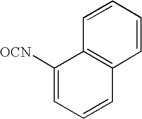 | C |
| 21.46 | 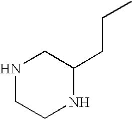 | 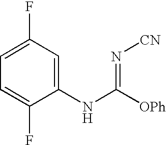 | B | 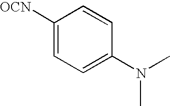 | C |
| 21.47 | 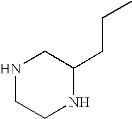 | 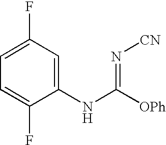 | B | 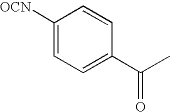 | C |
| 21.48 | 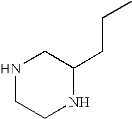 | 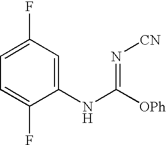 | B | 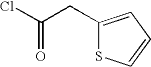 | D |
| 21.49 | 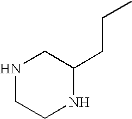 | 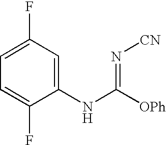 | B | 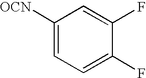 | C |
| 21.50 | 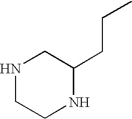 | 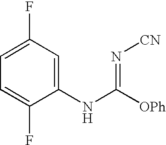 | B | 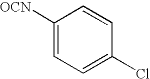 | C |
| 21.51 | 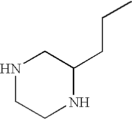 | 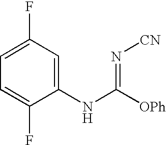 | B | 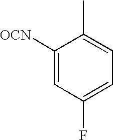 | C |
| 21.52 | 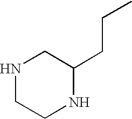 | 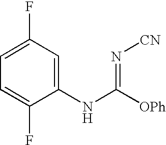 | B | 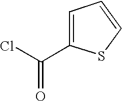 | D |
| 21.53 | 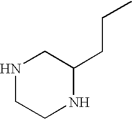 | 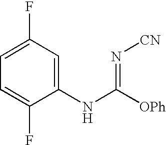 | B | 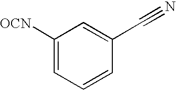 | C |
| 21.54 | 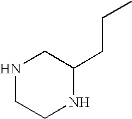 | 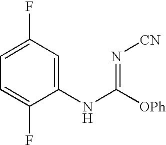 | B | 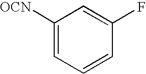 | C |
| 21.55 | 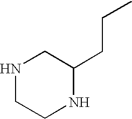 | 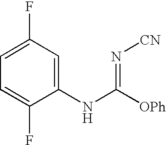 | B | 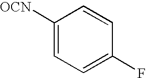 | C |
| 21.56 | 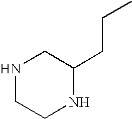 | 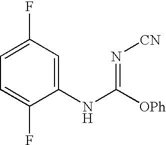 | B | 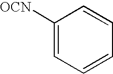 | C |
| 21.57 | 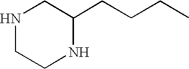 | 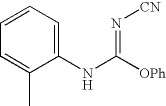 | B | 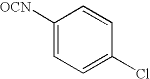 | C |
| 21.58 | 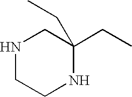 | 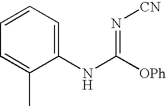 | B | 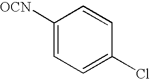 | C |
| 21.59 | 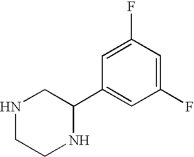 | 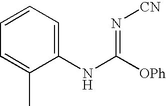 | B | 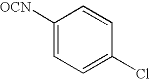 | C |
| 21.60 | 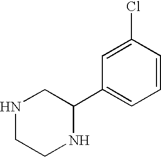 | 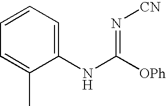 | B | 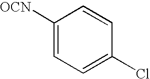 | C |
| 21.61 | 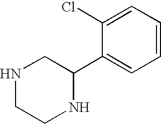 | 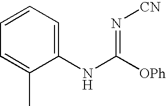 | B | 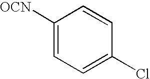 | C |
| 21.62 | 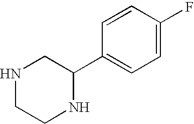 | 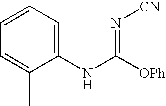 | B | 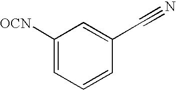 | C |
| 21.63 | 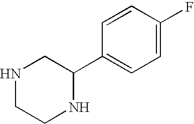 | 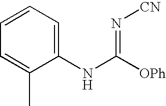 | B | 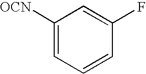 | C |
| 21.64 | 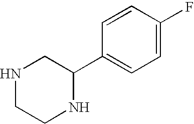 | 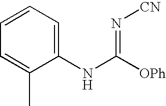 | B | 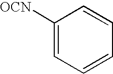 | C |
| 21.65 | 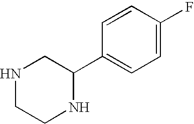 | 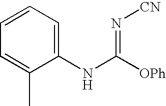 | B | 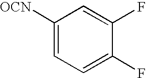 | C |
| 21.66 | 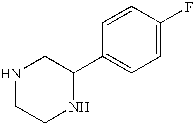 | 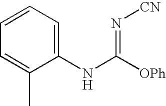 | B | 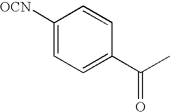 | C |
| 21.67 | 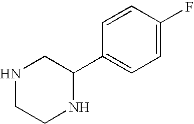 | 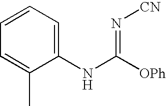 | B | 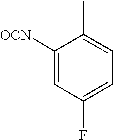 | C |
| 21.68 | 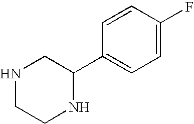 | 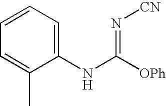 | B | 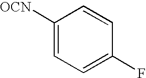 | C |
| 21.69 | 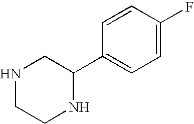 | 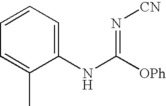 | B | 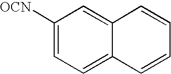 | C |
| 21.70 | 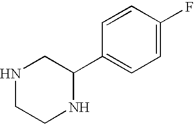 | 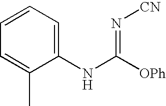 | B | 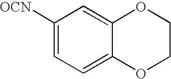 | C |
| 21.71 | 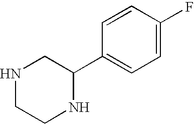 | 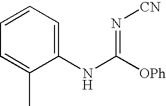 | B | 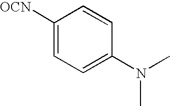 | C |
| 21.72 | 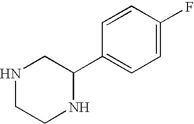 | 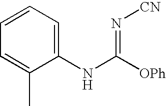 | B | 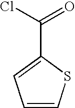 | D |
| 21.73 | 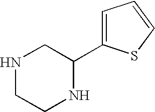 | 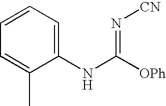 | B | 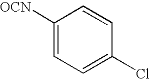 | C |
| 21.74 | 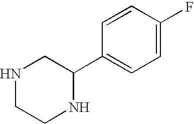 | 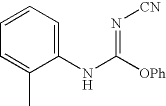 | B | 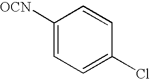 | C |
| 21.75 | 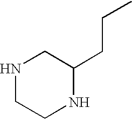 | 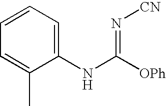 | B | 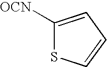 | C |
| 21.76 | 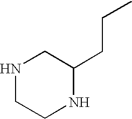 | 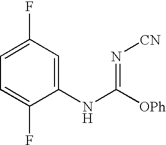 | B | 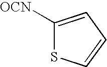 | C |
| 21.77 | 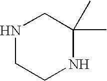 | 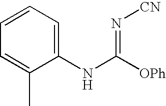 | B | 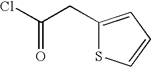 | D |
| 21.78 | 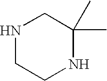 | 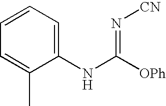 | B | 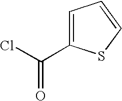 | D |
| 21.79 | 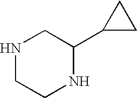 | 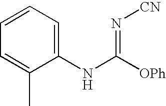 | B | 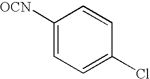 | D |
| 21.80 | 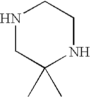 | 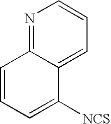 | G | 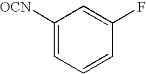 | C |
| 21.81 | 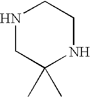 | 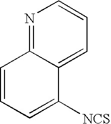 | G | 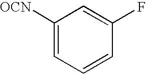 | C |
| 21.82 | 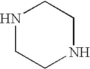 | 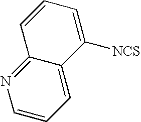 | G | 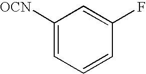 | C |
| 21.83 | 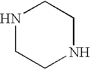 | 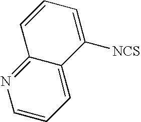 | G | 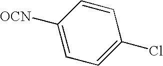 | C |

| | | Rt/min | |
|---|---|---|---|
| Ex # | Product | (method) | m/z |
| 21.1 | 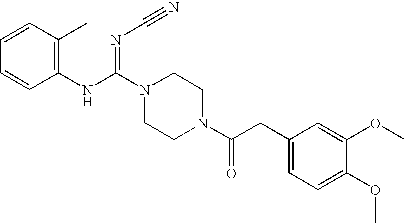 | 1.80 (e) | 422 (M + H)⁺ |
| 21.2 | 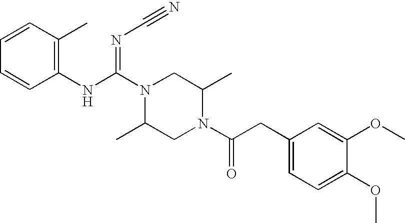 | 2.12 (i) | 450 (M + H)⁺ |
| 21.3 | 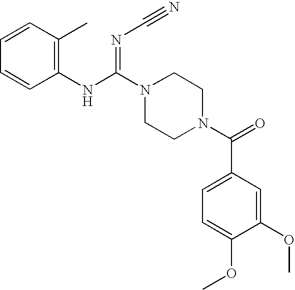 | 1.80 (i) | 408 (M + H)⁺ |
| 21.4 | 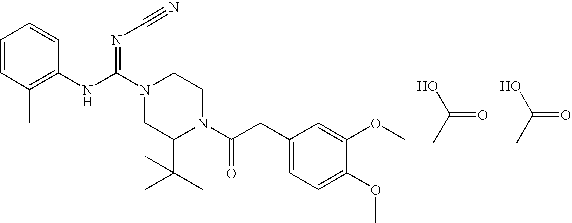 | 2.75 (i) | 478 (M + H)⁺ |
| 21.5 | 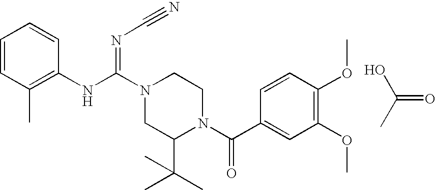 | 2.00 (i) | 464 (M + H)⁺ |
| 21.6 | 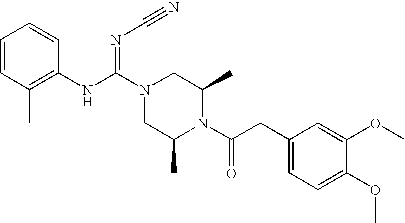 | 1.75 (i) | 450 (M + H)⁺ |
| 21.7 | 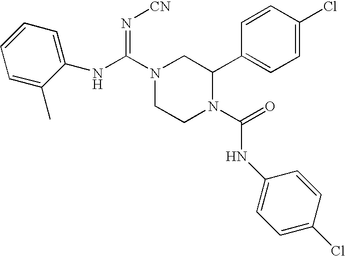 | 2.96 (i) | 507/509 (M + H)⁺ |
| 21.8 | 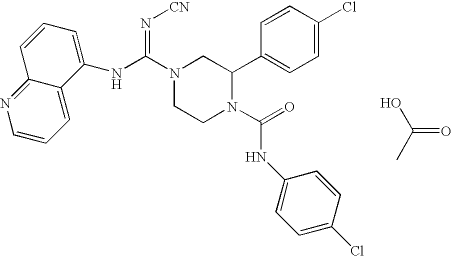 | 2.59 (i) | 544/546 (M + H)⁺ |
| 21.9 | 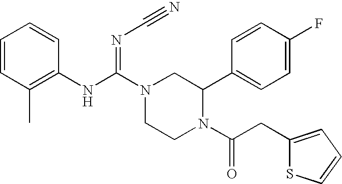 | 2.50 (i) | 462 (M + H)⁺ |
| 21.10 | 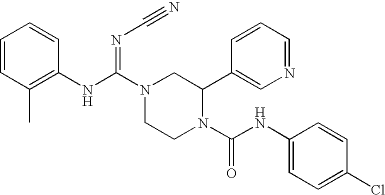 | 2.25 (i) | 474 (M + H)⁺ |
| 21.11 | 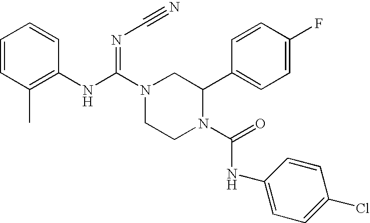 | 2.81 (i) | 491 (M + H)⁺ |
| 21.12 | 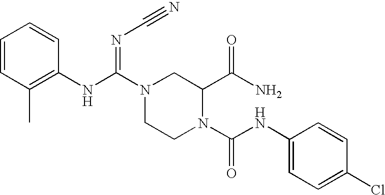 | 2.10 (g) | 440 (M + H)⁺ |
| 21.13 | 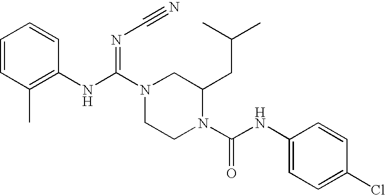 | 2.62 (g) | 427 (M + H)⁺ |
| 21.14 | 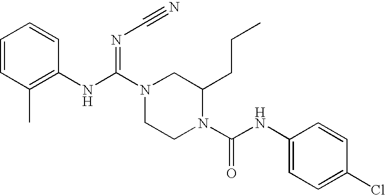 | 2.62 (g) | 428 (M + H)⁺ |
| 21.15 | 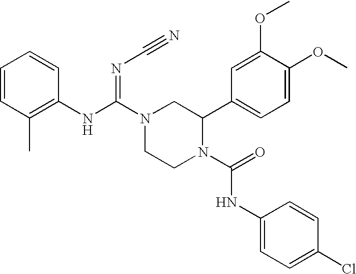 | 1.50 (i) | 533 (M + H)⁺ |
| 21.16 | 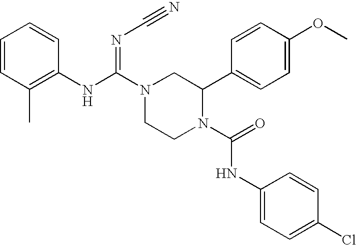 | 2.90 (i) | 503 (M + H)⁺ |
| 21.17 | 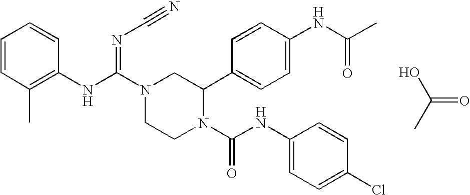 | 2.35 (i) | 530 (M + H)⁺ |
| 21.18 | 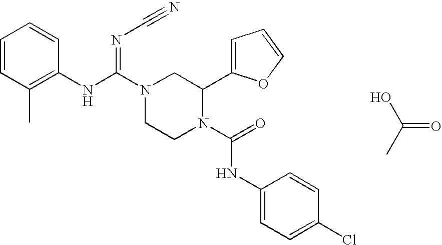 | 2.78 (i) | 463 (M + H)⁺ |
| 21.19 | 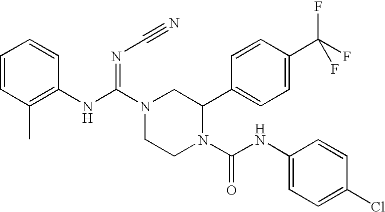 | 3.18 (i) | 541 (M + H)⁺ |
| 21.20 | 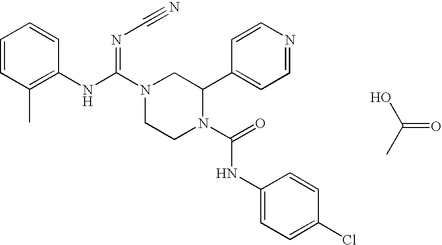 | 2.88 (i) | 474 (M + H)⁺ |
| 21.21 | 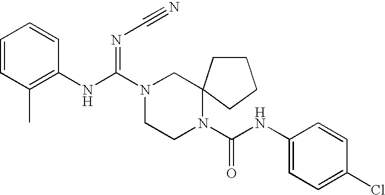 | 3.10 (g) | 451 (M + H)⁺ |
| 21.22 | 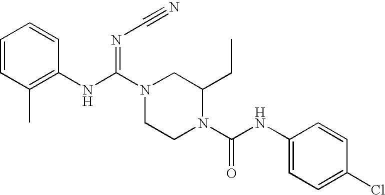 | 2.75 (g) | 425 (M + H)⁺ |
| 21.23 | 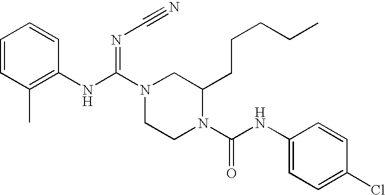 | 3.35 (g) | 467 (M + H)⁺ |
| 21.24 | 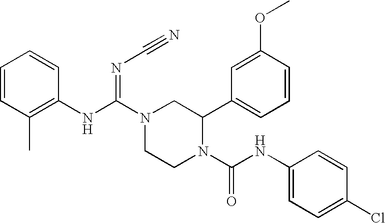 | 2.13 (i) | 503 (M + H)⁺ |
| 21.25 | 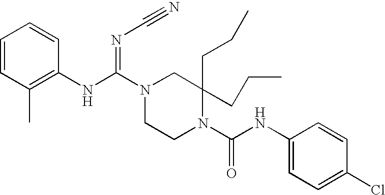 | 2.33 (d) | 481 (M + H)⁺ |
| 21.26 | 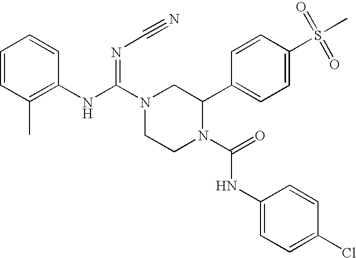 | 1.92 (d) | 551 (M + H)⁺ |
| 21.27 | 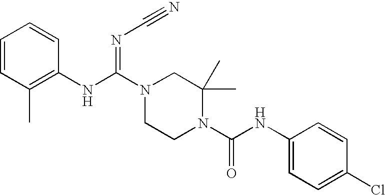 | 2.05 (d) | 425 (M + H)⁺ |
| 21.28 | 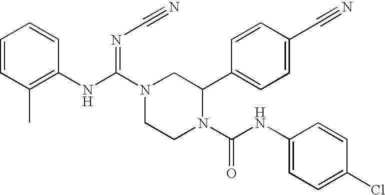 | 2.07 (d) | 498 (M + H)⁺ |
| 21.29 | 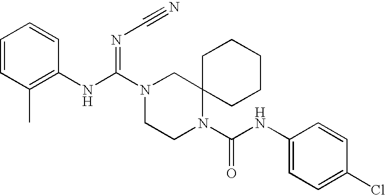 | 2.25 (d) | 465 (M + H)⁺ |
| 21.30 | 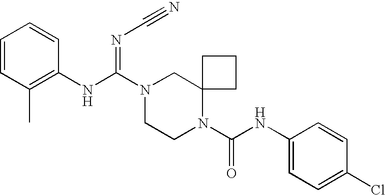 | 2.13 (d) | 437 (M + H)⁺ |
| 21.31 | 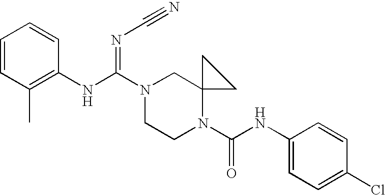 | 1.97 (d) | 423 (M + H)⁺ |
| 21.32 | 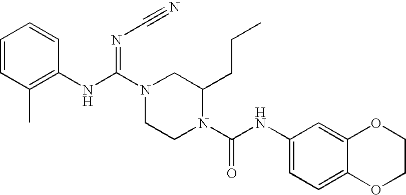 | 1.75 (d) | 463 (M + H)⁺ |
| 21.33 | 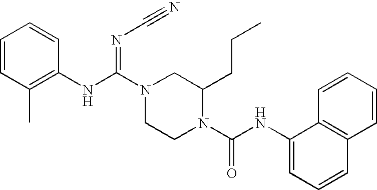 | 1.93 (d) | 455 (M + H)⁺ |
| 21.34 | 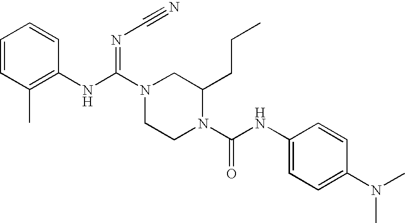 | 1.82 (d) | 447 (M + H)⁺ |
| 21.35 | 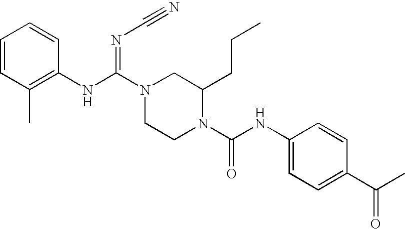 | 1.72 (d) | 448 (M + H)⁺ |
| 21.36 | 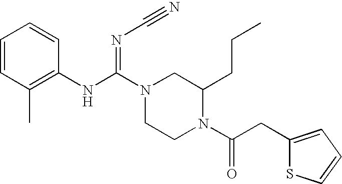 | 1.80 (d) | 410 (M + H)⁺ |
| 21.37 | 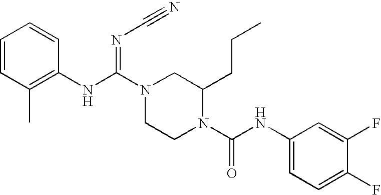 | 1.98 (d) | 441 (M + H)⁺ |
| 21.38 | 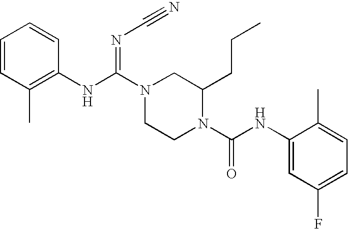 | 1.93 (d) | 437 (M + H)⁺ |
| 21.39 | 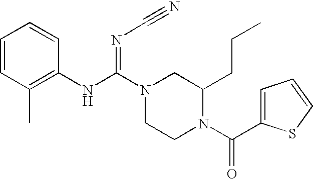 | 1.77 (d) | 396 (M + H)⁺ |
| 21.40 | 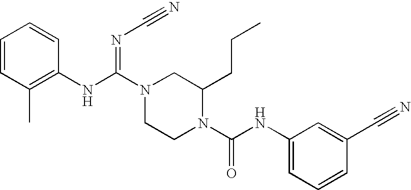 | 1.83 (d) | 430 (M + H)⁺ |
| 21.41 | 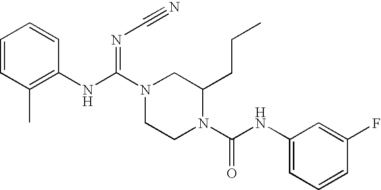 | 1.92 (d) | 423 (M + H)⁺ |
| 21.42 | 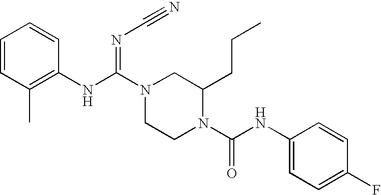 | 1.85 (d) | 423 (M + H)⁺ |
| 21.43 | 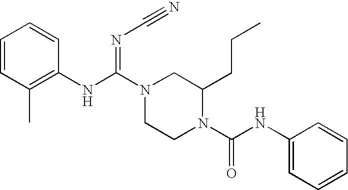 | 1.82 (d) | 405 (M + H)⁺ |
| 21.44 | 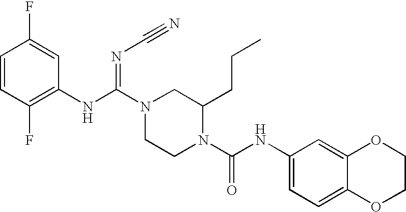 | 1.75 (d) | 485 (M + H)⁺ |
| 21.45 | 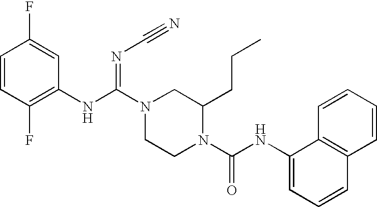 | 1.95 (d) | 477 (M + H)⁺ |
| 21.46 | 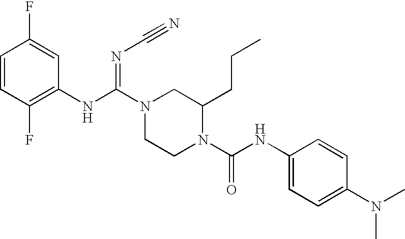 | 1.80 (d) | 470 (M + H)⁺ |
| 21.47 | 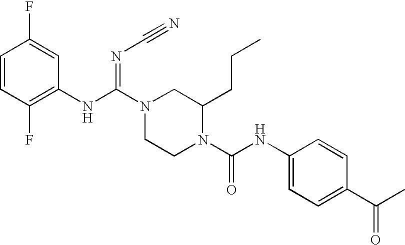 | 1.72 (d) | 469 (M + H)⁺ |
| 21.48 | 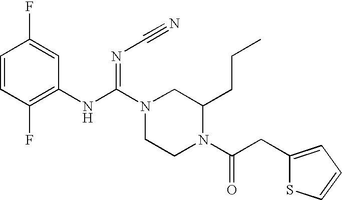 | 1.82 (d) | 432 (M + H)⁺ |
| 21.49 | 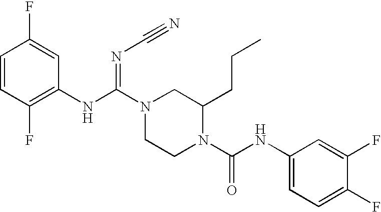 | 1.98 (d) | 463 (M + H)⁺ |
| 21.50 | 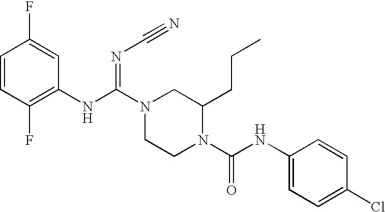 | 2.05 (d) | 461 (M + H)⁺ |
| 21.51 | 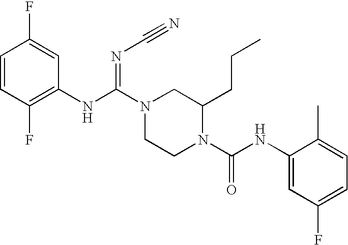 | 1.95 (d) | 459 (M + H)⁺ |
| 21.52 | 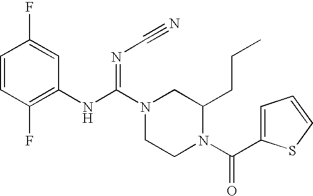 | 1.77 (d) | 418 (M + H)⁺ |
| 21.53 | 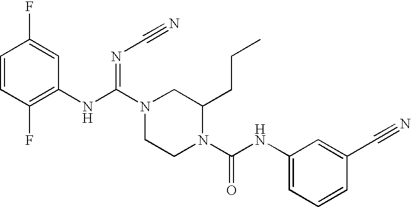 | 1.85 (d) | 452 (M + H)⁺ |
| 21.54 | 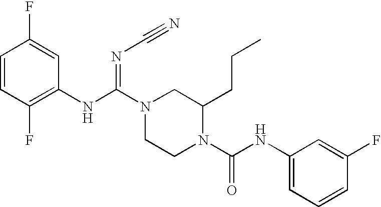 | 1.97 (d) | 445 (M + H)⁺ |
| 21.55 | 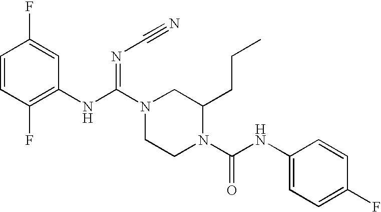 | 1.88 (d) | 445 (M + H)⁺ |
| 21.56 | 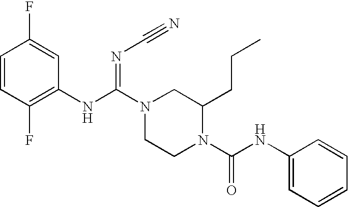 | 1.85 (d) | 427 (M + H)⁺ |
| 21.57 | 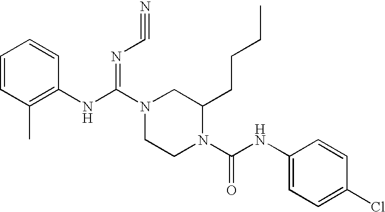 | 5.84 (a) | 453 (M + H)⁺ |
| 21.58 | 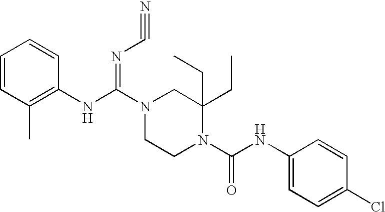 | 2.02 (d) | 453 (M + H)⁺ |
| 21.59 | 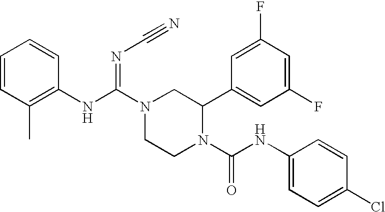 | 2.05 (d) | 509 (M + H)⁺ |
| 21.60 | 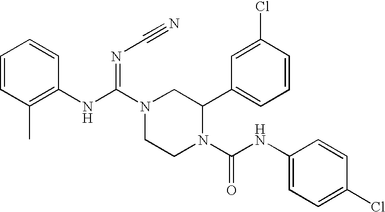 | 2.30 (d) | 505 (M − H)⁻ |
| 21.61 | 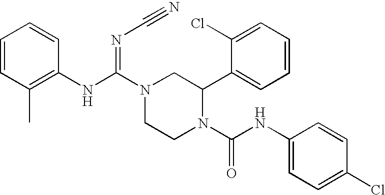 | 2.23 (d) | 507 (M + H)⁺ |
| 21.62 | 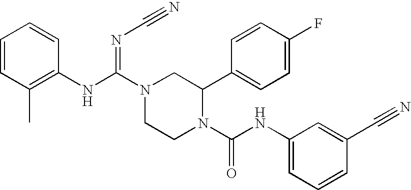 | 1.85 (d) | 480 (M − H)⁻ |
| 21.63 | 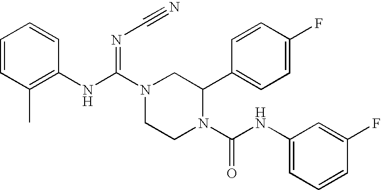 | 1.94 (d) | 475 (M + H)⁺ |
| 21.64 | 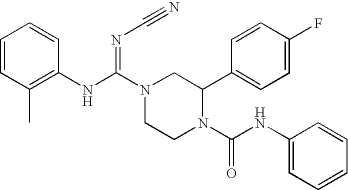 | 1.79 (d) | 457 (M + H)⁺ |
| 21.65 | 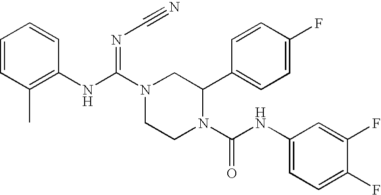 | 1.90 (d) | 493 (M + H)⁺ |
| 21.66 | 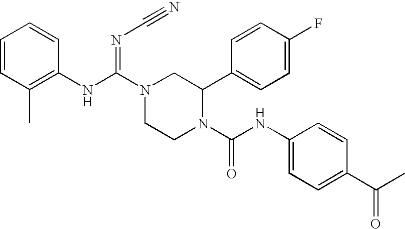 | 1.66 (d) | 499 (M + H)⁺ |
| 21.67 | 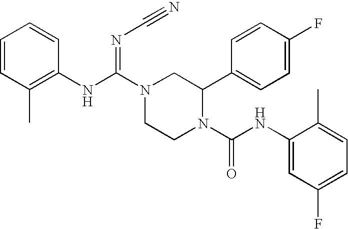 | 1.89 (d) | 489 (M + H)⁺ |
| 21.68 | 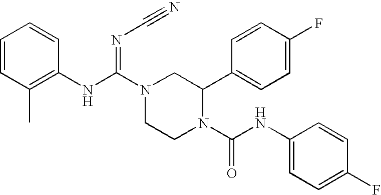 | 1.82 (d) | 475 (M + H)⁺ |
| 21.69 | 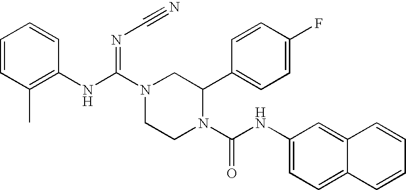 | 1.91 (d) | 507 (M + H)⁺ |
| 21.70 | 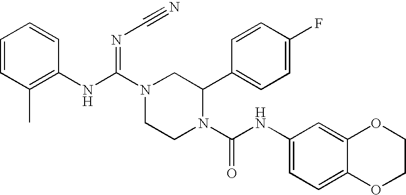 | 1.70 (d) | 515 (M + H)⁺ |
| 21.71 | 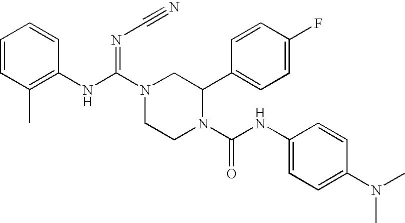 | 1.78 (d) | 500 (M + H)⁺ |
| 21.72 | 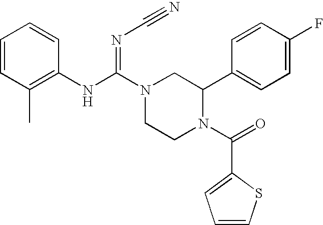 | 1.77 (d) | 448 (M + H)⁺ |
| 21.73 | 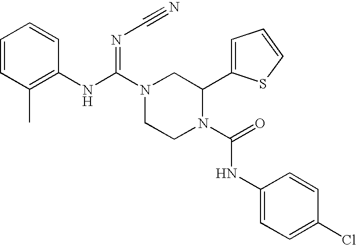 | 1.99 (d) | 479 (M + H)⁺ |
| 21.74 | 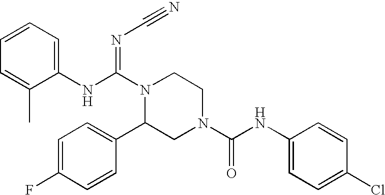 | 2.30 (d) | 491 (M + H)⁺ |
| 21.75 | 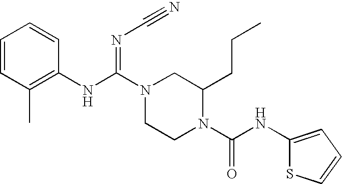 | 1.85 (d) | 411 (M + H)⁺ |
| 21.76 | 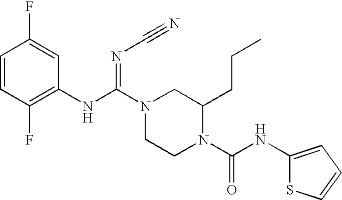 | 1.85 (d) | 433 (M + H)+ |
| 21.77 | 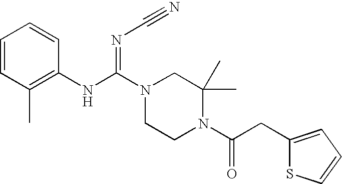 | 1.75 (d) | 396 (M + H)⁺ |
| 21.78 | 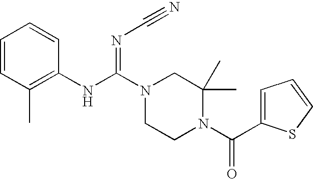 | 1.65 (d) | 382 (M + H)⁺ |
| 21.79 | 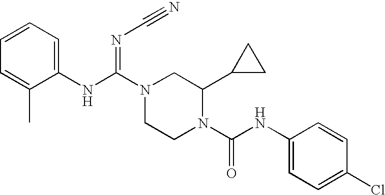 | 2.03 (d) | 437 (M + H)⁺ |
| 21.80 | 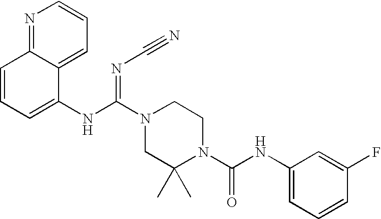 | 1.64 (d) | 446 (M + H)⁺ |
| 21.81 | 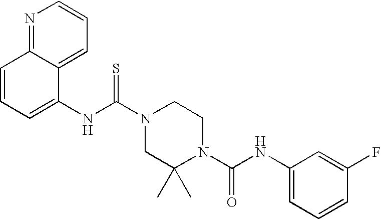 | 1.74 (d) | 438 (M + H)⁺ |
| 21.82 | 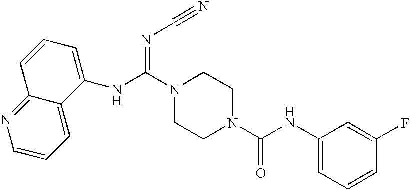 | 1.57 (b) | 418 (M + H)⁺ |
| 21.83 | 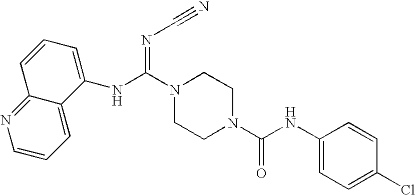 | 1.65 (b) | 434 (M + H)⁺ |

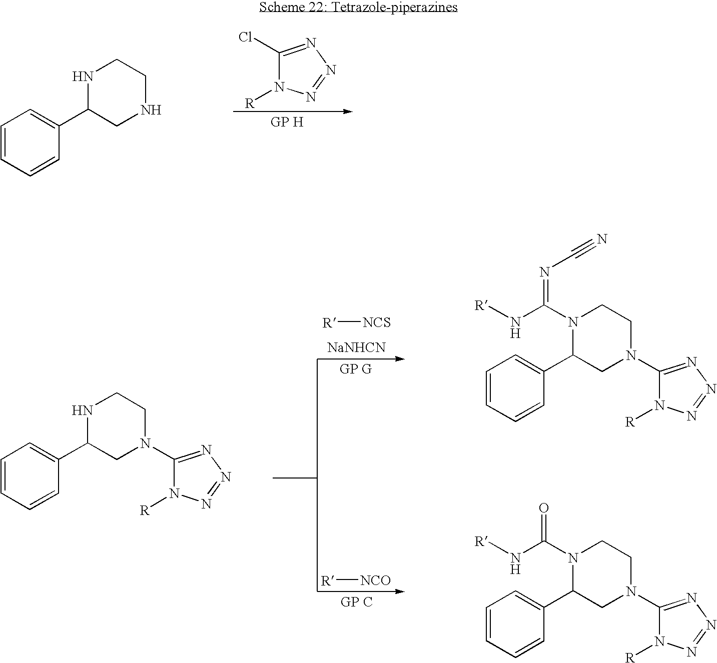

The compounds in table 22 were made according to general procedure H, followed by either general procedure C or G, as illustrated in scheme 22. The specific procedure used for each compound is listed in table 22.

TABLE 22
Tetrazole-piperazines

| | | R′—NCS | | | | |
|---|---|---|---|---|---|---|
| | | or | | | Rt/min | |
| Ex # | Reagent | R′—NCO | GP | Product | (method) | m/z |
| 22.1 | 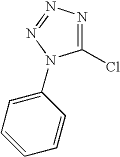 | 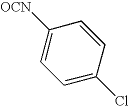 | C | 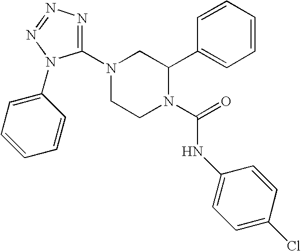 | 2.11 (d) | 460 (M + H)⁺ |
| 22.2 | 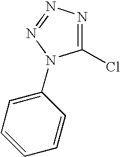 | 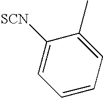 | G | 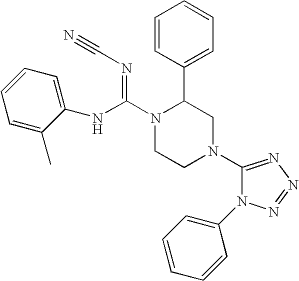 | 1.85 (d) | 464 (M + H)⁺ |
| 22.3 | 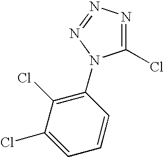 | 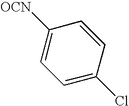 | C | 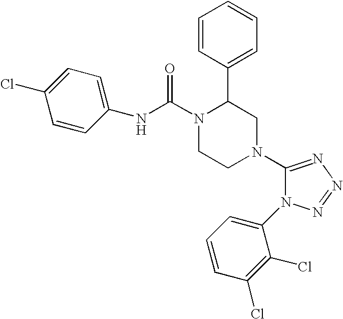 | 2.29 (d) | 528 (M + H)⁺ |
| 22.4 | 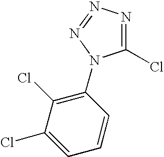 | 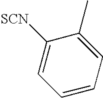 | G | 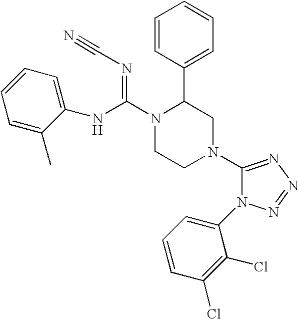 | 2.10 (d) | 532 (M + H)⁺ |

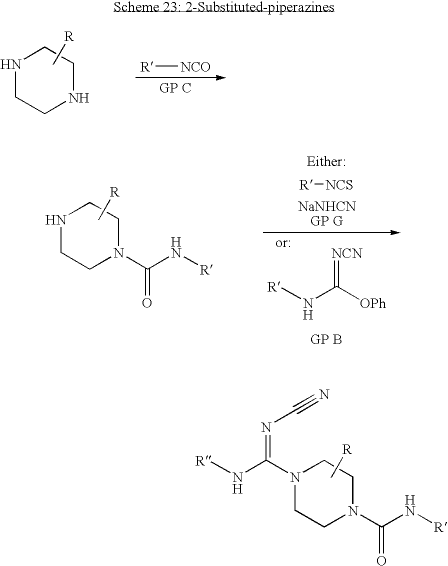

The compounds in table 23 were made according to general procedure C, followed by either general procedure B or G, as illustrated in scheme 23. The specific procedure used for each compound is listed in table 23.

TABLE 23
2-Substituted-piperazines

| Ex # | Piperazine | R′—NCO | 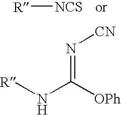 |
|---|---|---|---|
| 23.1 | 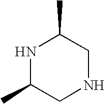 | 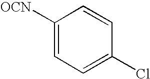 | 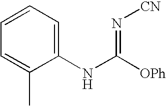 |
| 23.2 | 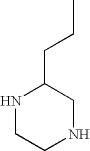 | 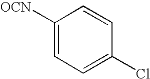 | 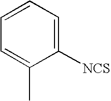 |
| 23.3 | 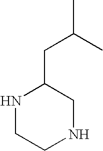 | 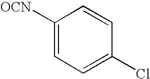 | 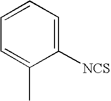 |
| 23.4 | 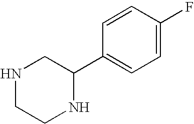 | 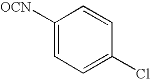 | 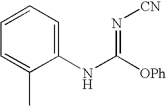 |
| 23.5 | 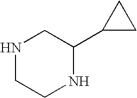 | 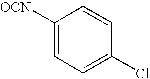 | 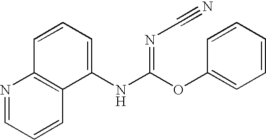 |
| 23.6 | 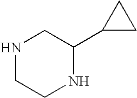 | 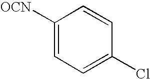 | 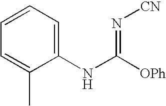 |
| 23.7 | 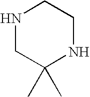 | 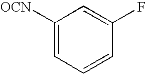 | 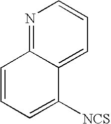 |
| 23.8 | 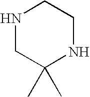 | 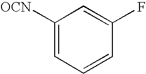 | 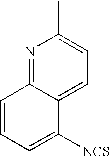 |

| | | | Rt/min | |
|---|---|---|---|---|
| Ex # | GP | Product | (method) | m/z |
| 23.1 | B | 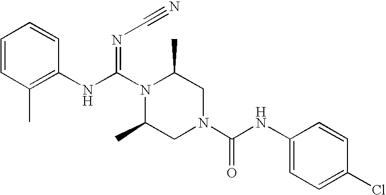 | 2.62 (d) | 425 (M + H)⁺ |
| 23.2 | G | 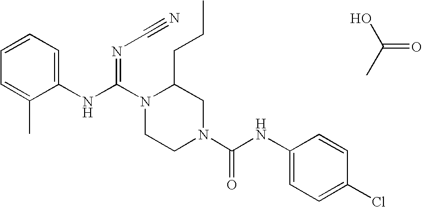 | 1.92 (d) | 439 (M + H)⁺ |
| 23.3 | G | 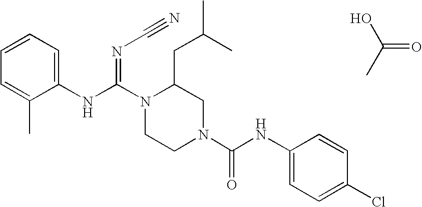 | 2.01 (d) | 453 (M + H)⁺ |
| 23.4 | B | 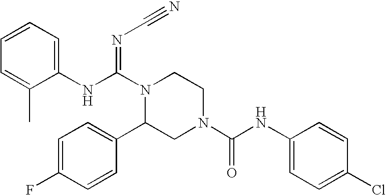 | 2.03 (d) | 491 (M + H)⁺ |
| 23.5 | B | 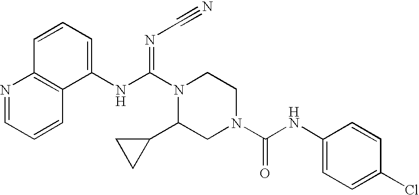 | 168 (d) | 474 (M + H)⁺ |
| 23.6 | B | 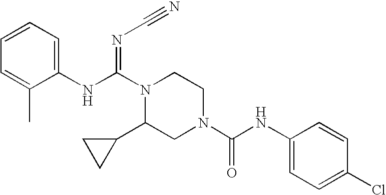 | 1.97 (d) | 437 (M + H)⁺ |
| 23.7 | G | 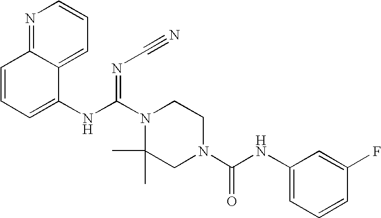 | 2.35 (d) | 446 (M + H)⁺ |
| 23.8 | G | 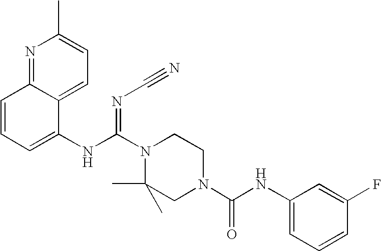 | 2.10 (d) | 460 (M + H)⁺ |

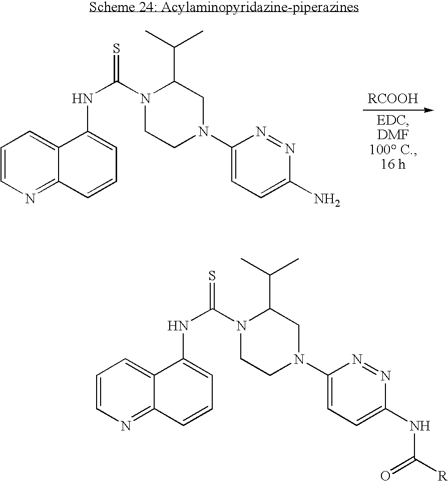

The compounds in table 24 were made as illustrated in scheme 24.

TABLE 24
Acylaminopyridazine-piperazines

| | | | Rt/min | |
|---|---|---|---|---|
| Ex # | R—COOH | Product | (method) | m/z |
| 24.1 | 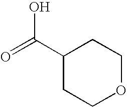 | 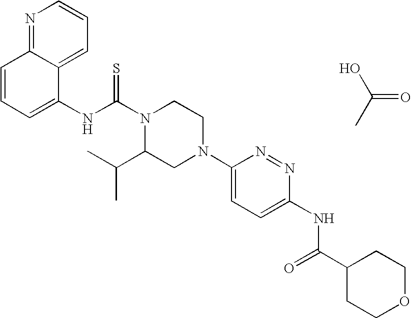 | 1.56 (d) | 520 (M + H)⁺ |
| 24.2 | 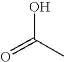 | 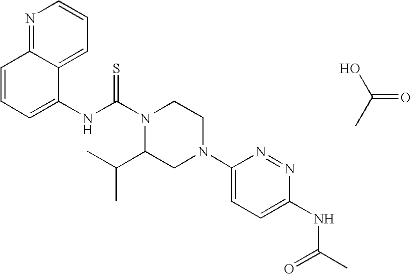 | 1.64 (d) | 492 (M + H)⁺ |
| 24.3 | 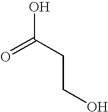 | 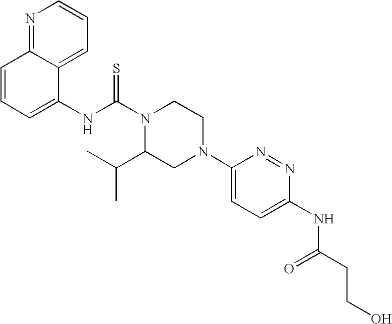 | 1.46 (d) | 480 (M + H)⁺ |

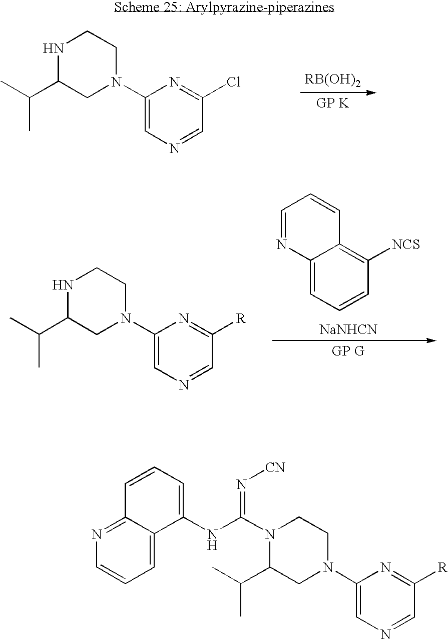

The compounds in table 25 were made according to general procedure K, followed by general procedure G, as illustrated in scheme 25.

TABLE 25
Arylpyrazine-piperazines

| | | | Rt/min | |
|---|---|---|---|---|
| Ex # | R—B(OH)₂ | Product | (method) | m/z |
| 25.1 | 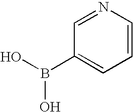 | 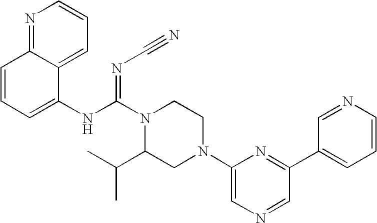 | 1.68 (d) | 478 (M + H)⁺ |
| 25.2 | 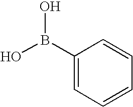 | 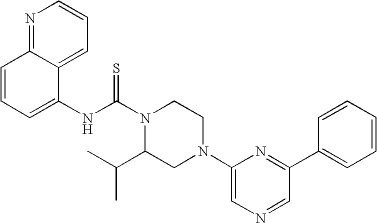 | 1.93 (d) | 469 (M + H)⁺ |
| 25.3 | 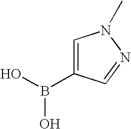 | 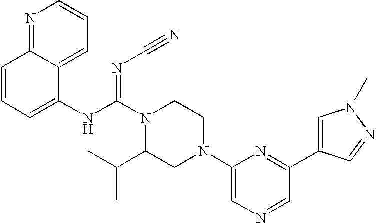 | 1.58 (d) | 481 (M + H)⁺ |
| 25.4 | 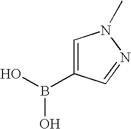 | 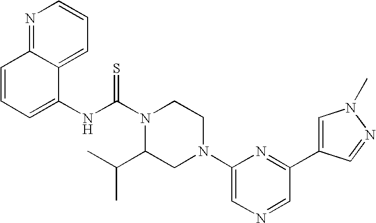 | 1.70 (d) | 473 (M + H)⁺ |

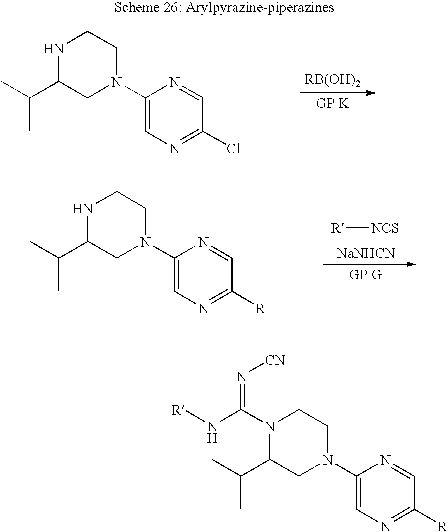

The compounds in table 26 were made according to general procedure K, followed by general procedure G, as illustrated in scheme 26.

TABLE 26
Arylpyrazine-piperazines

| | | | | Rt/min | |
|---|---|---|---|---|---|
| Ex # | R—NCS | R′—B(OH)₂ | Structure | (method) | m/z |
| 26.1 | 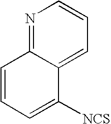 | 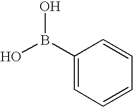 | 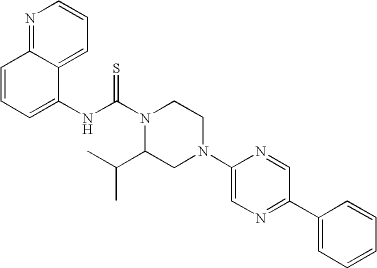 | 2.08 (d) | 469 (M + H)⁺ |
| 26.2 | 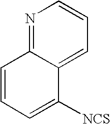 | 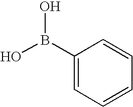 | 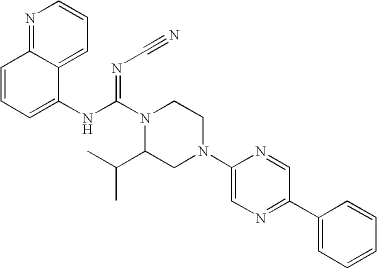 | 4.77 (d) | 478 (M + H)⁺ |
| 26.3 | 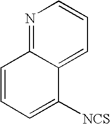 | 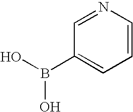 | 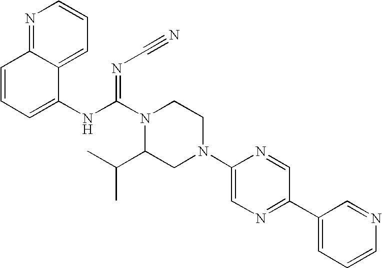 | 1.85 (d) | 470 (M + H)⁺ |
| 26.4 | 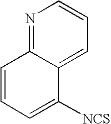 | 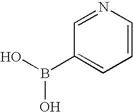 | 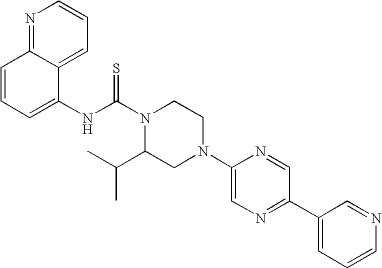 | 1.69 (d) | 479 (M + H)⁺ |
| 26.5 | 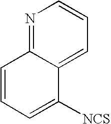 | 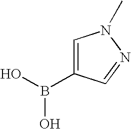 | 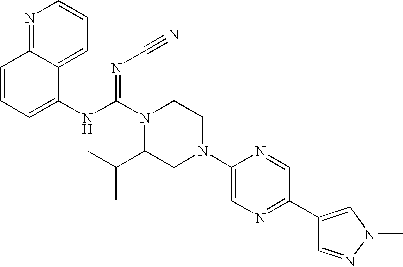 | 1.58 (d) | 481 (M + H)⁺ |
| 26.6 | 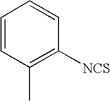 | 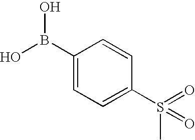 | 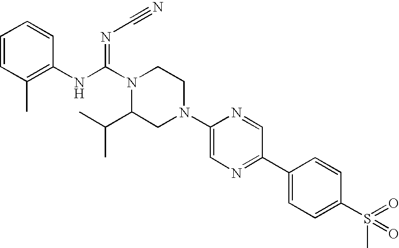 | 2.71 (a) | 518 (M + H)⁺ |
| 26.7 | 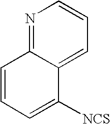 | 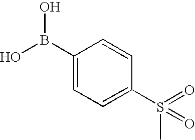 | 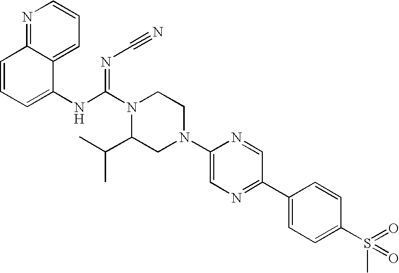 | 2.42 (a) | 555 (M + H)⁺ |
| 26.8 | 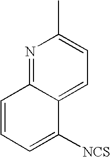 | 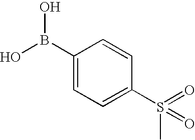 | 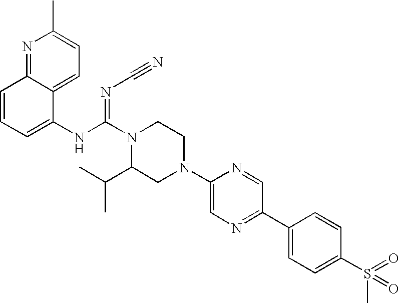 | 2.45 (a) | 569 (M + H)⁺ |

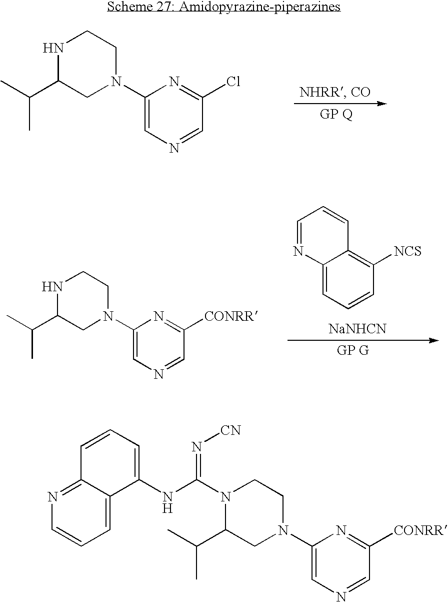

The compounds in table 27 were made according to general procedure Q, followed by general procedure G, as illustrated in scheme 27.

TABLE 27
Amidopyrazine-piperazines

| | | | Rt/min | |
|---|---|---|---|---|
| Ex # | NHRR′ | Product | (method) | m/z |
| 27.1 | 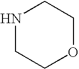 | 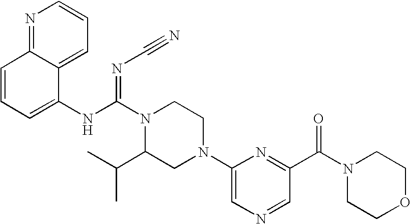 | 1.49 (d) | 514 (M + H)⁺ |
| 27.2 | 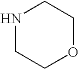 | 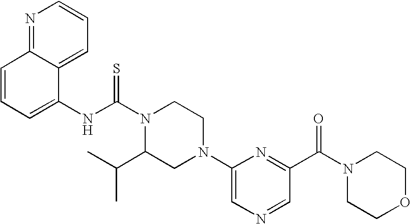 | 1.57 (d) | 506 (M + H)⁺ |
| 27.3 | 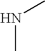 | 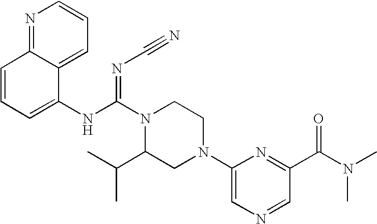 | 2.41 (a) | 472 (M + H)⁺ |
| 27.4 | 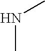 | 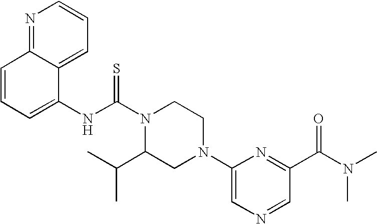 | 2.66 (a) | 464 (M + H)⁺ |

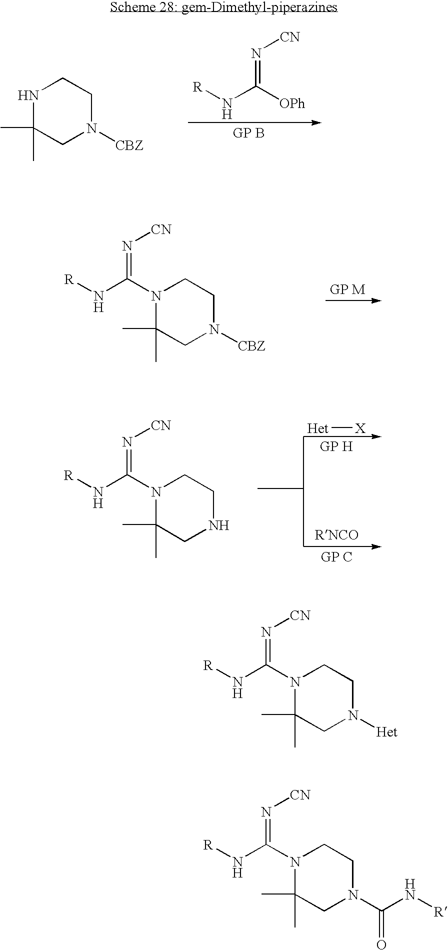

The compounds in table 28 were made according to general procedure B, followed by general procedure M and either general procedure C or H, as illustrated in scheme 28. The specific procedure used for each compound is listed in table 28.

TABLE 28
gem-Dimethyl-piperazines

| Ex # | 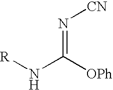 | Het-X or R′—NCO | GP | Product |
|---|---|---|---|---|
| 28.1 | 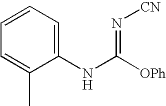 | 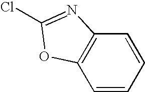 | H | 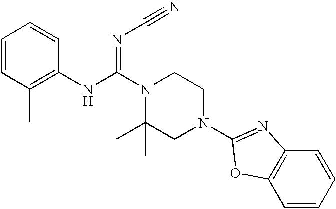 |
| 28.2 | 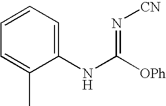 | 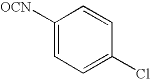 | C | 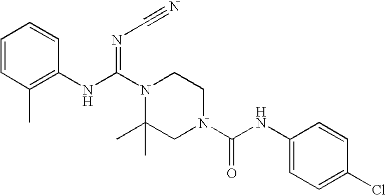 |
| 28.3 | 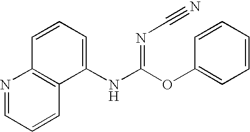 | 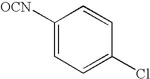 | C | 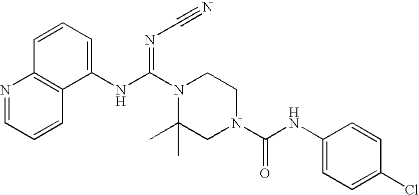 |
| 28.4 | 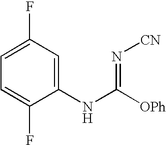 | 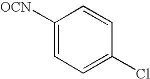 | C | 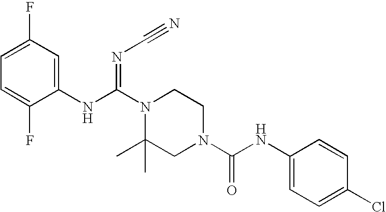 |
| 28.5 | 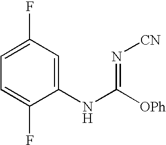 | 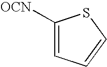 | C | 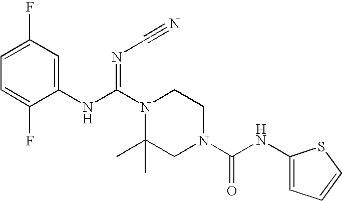 |
| 28.6 | 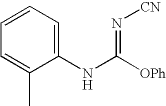 | 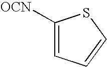 | C | 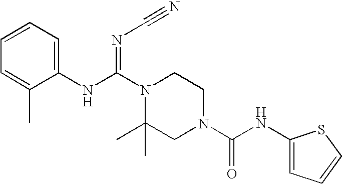 |
| 28.7 | 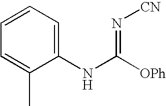 | 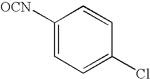 | C | 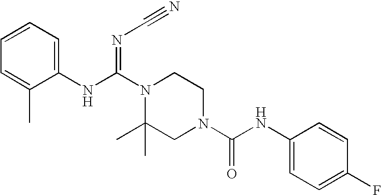 |
| 28.8 | 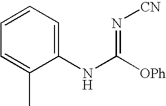 | 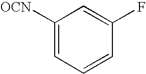 | C | 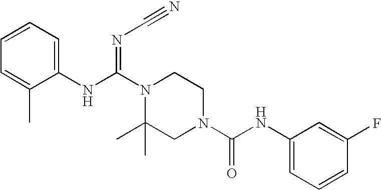 |
| 28.9 | 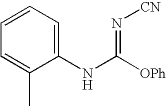 | 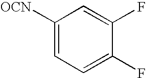 | C | 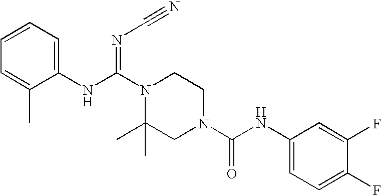 |

| | Rt/min | |
|---|---|---|
| Ex # | (method) | m/z |
| 28.1 | 1.98 (d) | 389 (M + H)⁺ |
| 28.2 | 1.92 (d) | 425 (M + H)⁺ |
| 28.3 | 1.67 (d) | 460 (M − H)⁻ |
| 28.4 | 1.93 (d) | 445 (M − H)⁻ |
| 28.5 | 1.7 (d) | 417 (M − H)⁻ |
| 28.6 | 1.62 (d) | 495 (M − H)⁻ |
| 28.7 | 1.77 (d) | 407 (M − H)⁻ |
| 28.8 | 1.85 (d) | 407 (M − H)⁻ |
| 28.9 | 1.88 (d) | 425 (M − H)⁻ |

Scheme 29: Single Enantiomers

The entries in table 29 are single enantiomers of racemic compounds described above. They were prepared like their racemic analogs, but starting from enantiomerically pure 2R or 2S-isopropypiperazine.

TABLE 29
Single Enantiomers

| | | Rt/min | |
|---|---|---|---|
| Ex # | Product | (method) | m/z |
| 29.1 | 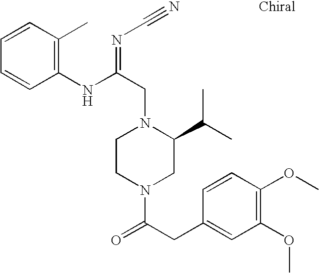 | 2.70 (i) | 478 (M + H)⁺ |
| 29.2 | 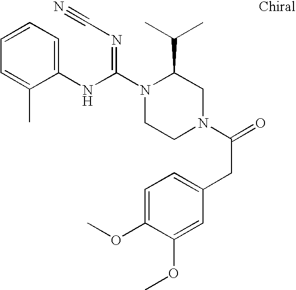 | 2.15 (i) | 464 (M + H)⁺ |
| 29.3 | 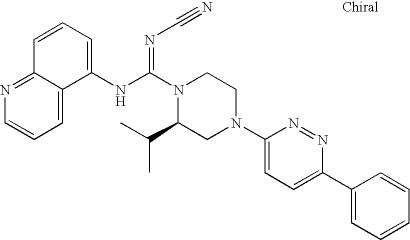 | 1.8 (a) | 477 (M + H)⁺ |
| 29.4 | 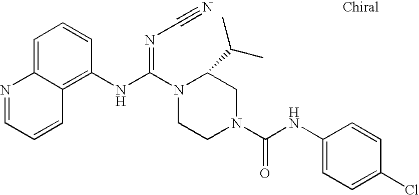 | 2.55 (a) | 476 (M + H)⁺ |
| 29.5 | 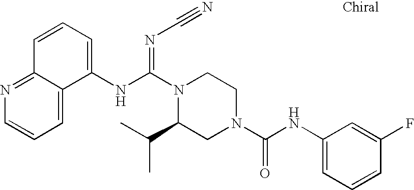 | 2.43 (a) | 460 (M + H)⁺ |
| 29.6 | 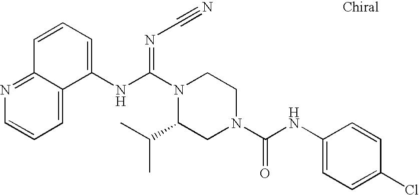 | 2.28 (a) | 476 (M + H)⁺ |
| 29.7 | 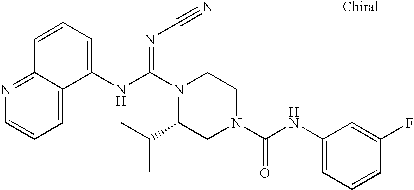 | 1.57 (d) | 460 (M + H)⁺ |
| 29.8 | 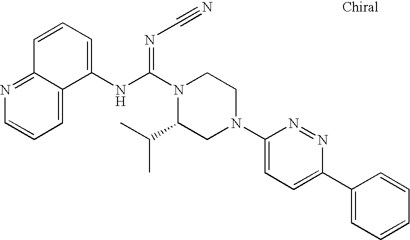 | 1.70 (d) | 477 (M + H)⁺ |
| 29.9 | 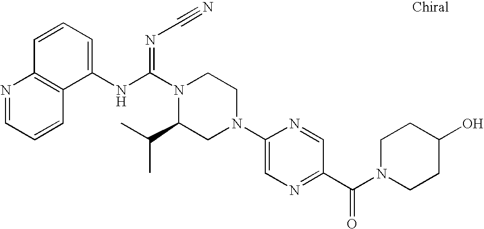 | 1.48 (b) | 528 (M + H)⁺ |
| 29.10 | 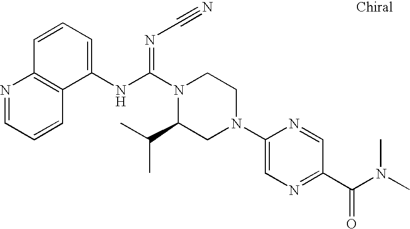 | 1.58 (b) | 472 (M + H)⁺ |
| 29.11 | 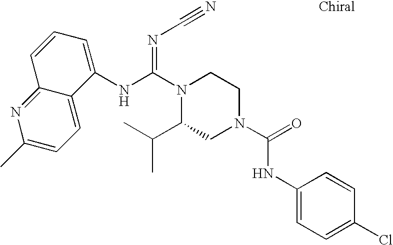 | 1.53 (d) | 490 (M + H)⁺ |
| 29.12 | 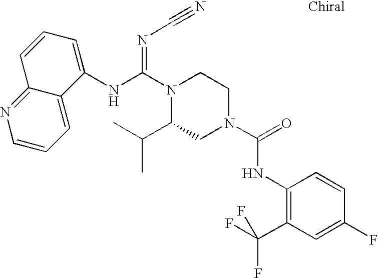 | 1.61 (d) | 528 (M + H)⁺ |
| 29.13 | 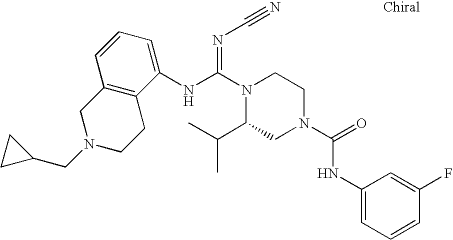 | 1.77 (d) | 518 (M + H)⁺ |
| 29.14 | 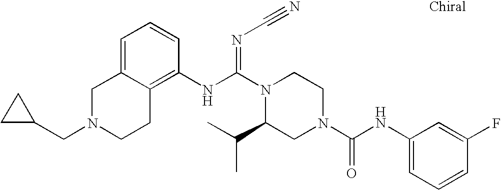 | 1.82 (b) | 518 (M + H)⁺ |
| 29.15 | 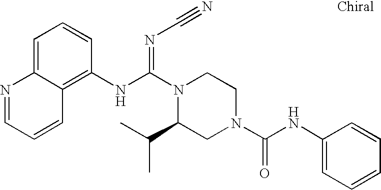 | 2.01 (b) | 520 (M + H)⁺ |
| 29.16 | 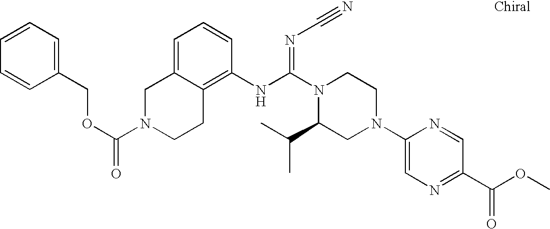 | 2.11 (b) | 597 (M + H)⁺ |
| 29.17 | 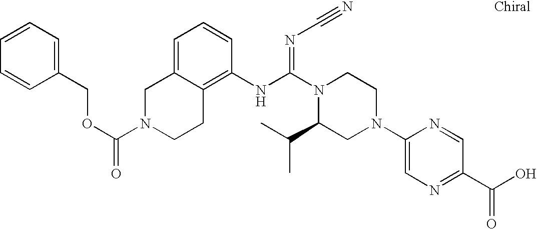 | 1.78 (b) | 581 (M − H)⁻ |
| 29.18 | 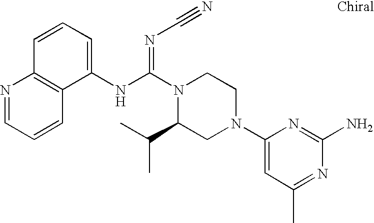 | 1.34 (b) | 430 (M + H)⁺ |
| 29.19 | 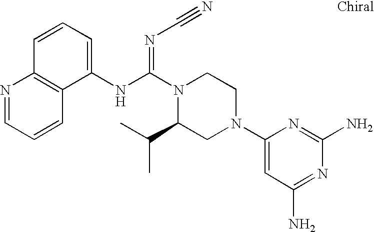 | 1.36 (b) | 431 (M + H)⁺ |
| 29.20 | 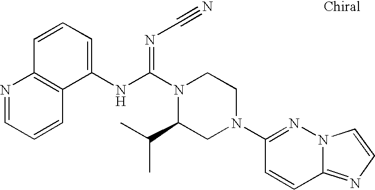 | 1.72 (b) | 440 (M + H)⁺ |
| 29.21 | 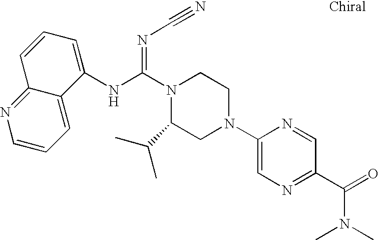 | 1.46 (d) | 472 (M + H)⁺ |
| 29.22 | 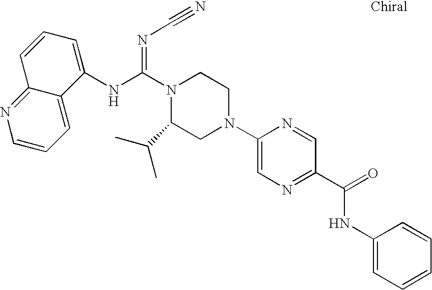 | 2.11 (d) | 520 (M + H)⁺ |
| 29.23 | 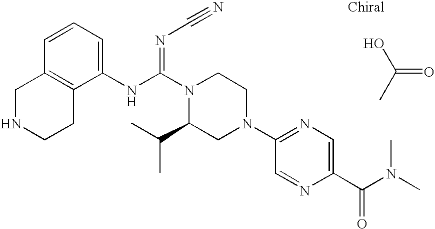 | 1.37 (b) | 476 (M + H)⁺ |
| 29.24 | 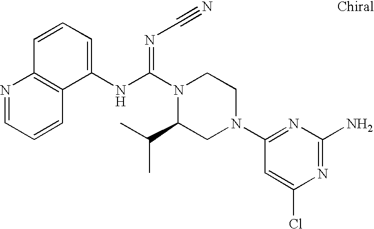 | 1.73 (b) | 450 (M + H)⁺ |
| 29.25 | 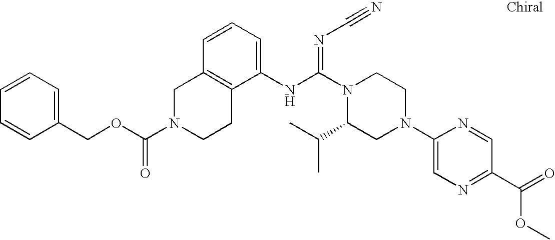 | 2.11 (b) | 596 (M + H)⁺ |
| 29.26 | 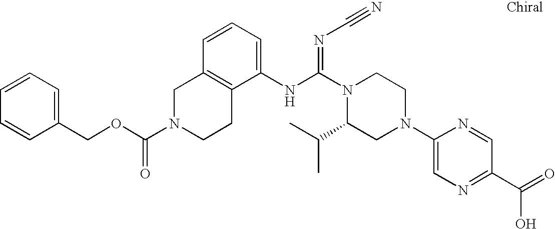 | 1.70 (b) | 581 (M − H)⁻ |
| 29.27 | 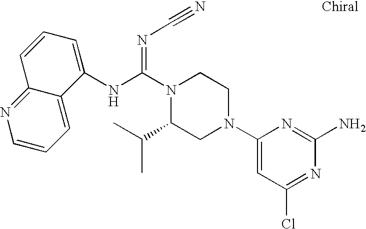 | 1.52 (d) | 450 (M + H)⁺ |
| 29.28 | 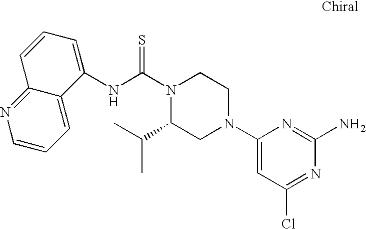 | 1.69 (d) | 442 (M + H)⁺ |
| 29.29 | 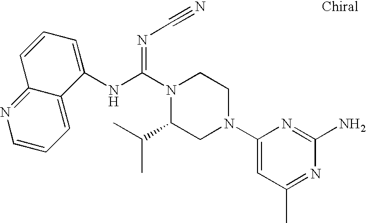 | 0.90 (d) | 430 (M + H)⁺ |
| 29.30 | 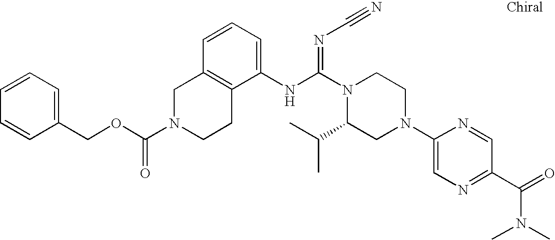 | 2.69 (b) | 608 (M − H)⁻ |
| 29.31 | 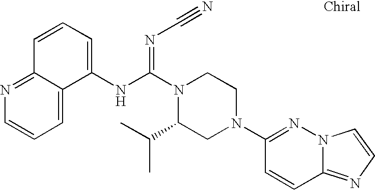 | 1.50 (a) | 440 (M + H)⁺ |
| 29.32 | 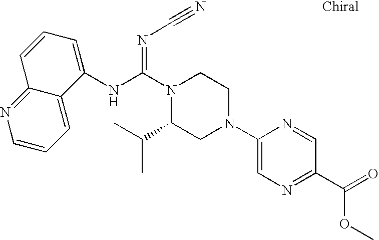 | 1.45 (d) | 459 (M + H)⁺ |
| 29.33 | 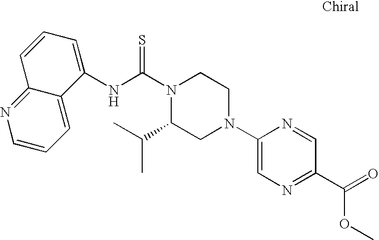 | 1.68 (d) | 451 (M + H)⁺ |
| 29.34 | 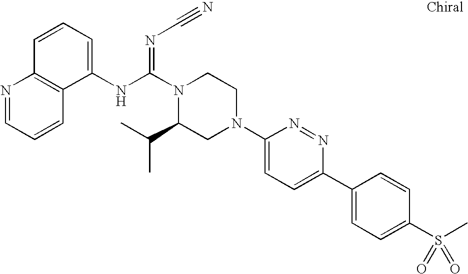 | 1.75 (b) | 555 (M + H)⁺ |
| 29.35 | 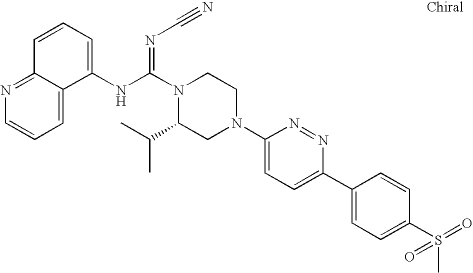 | 1.63 (d) | 555 (M + H)⁺ |
| 29.36 | 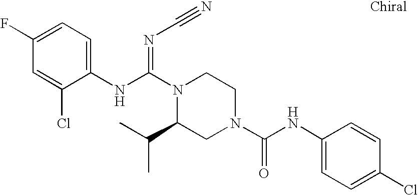 | 2.69 (a) | 477 (M + H)⁺ |
| 29.37 | 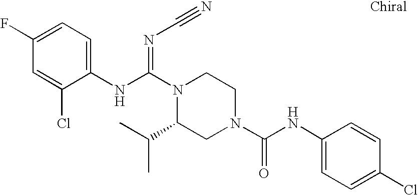 | 2.71 (a) | 477 (M + H)⁺ |

What is claimed:

1. A compound of Formula I 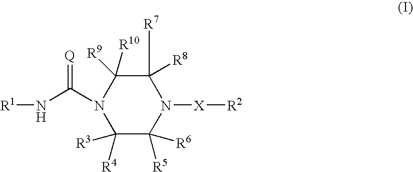
pharmaceutically acceptable salts thereof, metabolites thereof, isomers thereof, enantiomers thereof or prodrugs thereof wherein
Q is N—CN or S;
X is selected from the group consisting of a bond, C(O), C(O)—N(X¹), S(O)₂ and C(O)—N(X¹)—S(O)₂;
wherein X¹ is H or alkyl;
R¹ is selected from the optionally substituted group consisting of diphenylalkyl, alkoxy, alkoxycarbonylalkyl, alkyl, amino, aryl, arylalkyl, benzyloxy, cycloalkyl, cycloalkylalkyl, heteroaryl and heterocyclyl; or
R¹ is A-B wherein A is attached to the nitrogen of the amino and
A is —C(O)— or is selected from the optionally substituted group consisting of alkylidenyl, heterocyclyl, aryl and heteroaryl;
B is selected from the optionally substituted group consisting of —(CH₂)ₙ—C(O)—O(CH₂)ₙ-aryl, —(CH₂)ₙ—NR^{a}—C(O)—O(CH₂)ₙ-phenyl, —NR^{a}—C(O)—(CH₂)ₙ—NR^{a}R^{b}, —C(O)—O(CH₂)ₙ-aryl, C(O)—(CH₂)ₙ—C(O)—NR^{a}R^{b}, —C(O)—R^{c}—NR^{a}—C(O)-cycloalkyl, —NR^{a}—C(O)—(CH₂)_{c}ycloalkyl, —NR^{a}—C(O)—O(CH₂)ₙ-phenyl, alkoxy, alkyl-NR^{a}R^{b}, NR^{a}-alkyl-R^{a}R^{b}, aryl, aryloxy, benzyloxy, cycloalkyl, cycloalkylalkyl, heterocyclyl, heterocyclyoalkyl, heterocycloalkylamine and heteroaryl; or
R² is H or —(CH₂)ₙ—C(O)O-alkyl; or
R² is selected from the optionally substituted group consisting of alkyl, aryl, arylalkyl, cycloalkyl, heteroaryl and heterocyclyl; or
R² is Y-Z wherein Y is attached to X and
Y is selected from the group consisting of alkylidenyl, alkenyl, aryl, cycloalkenyl, heteroaryl, heterocyclyl and 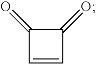
Z is —NR^{a}(CH₂)ₙ-Z¹⁰⁰, —NR^{a}—(CH₂)ₙ—C(O)-Z¹⁰⁰, —NR^{a}(CH₂)ₙ—S(O)₂-Z²⁰⁰, —NR^{a}—C(O)—(CH₂)ₙ—OH or is selected from the optionally substituted group consisting of —(CH₂)ₙ—NR^{a}—C(O)—O(CH₂)ₙ-aryl, -alkylNR^{a}R^{b}, aryl, aryloxy, benzyloxy, heteroaryl, heterocyclyl, —C(O)NR^{a}(CH₂), OH, —C(O)—O(CH₂)ₙ-aryl, —C(O)—R^{d}, —C(O)—NR^{a}R^{b}, —C(O)—NR^{a}—R^{d}, —O—R^{d} and —NR^{a}—R^{d};
wherein Z¹⁰⁰ is selected from the group consisting of OH, —NR^{a}R^{b}, alkoxy or optionally substituted heterocyclyl;
wherein Z²⁰⁰ is selected from the group consisting of alkyl and optionally substituted heterocyclyl and optionally substituted heterocyclylalkyl;
R³, R⁴, R⁵, R⁶, R⁷, R⁸, R⁹ and R¹⁰ are independently H, COOH, —C(O)—NH₂, —CH₂—O—(CH₂)ₘ—O—CH₂CH₂—OCH₃, —CH₂—O—CH₂—O—(CH₂)ₘ—OCH₃ or are independently selected from the optionally substituted group consisting of alkyl, alkenyl, alkynyl, alkoxyalkyl, cycloalkyl, aryl, heterocyclyl and heteroaryl; or
R⁷ and R⁸ taken together with the carbon to which they are attached to form a cycloalkyl attached to the piperazine; or
R⁹ and R¹⁰ taken together with the carbon atom to which they are attached form a cycloalkyl attached to the piperazine; or
R⁷ and R⁹ taken together with the carbon to which they are attached to form a cycloalkyl group attached to the piperazine; or
R⁸ and R¹⁰ taken together with the carbon atoms to which they are attached form a cycloalkyl group attached to the piperazine;
provided that R³ and R⁴ are not cycloalkyl, aryl, heteroaryl or heterocyclyl at the same time;
provided that R⁵ and R⁶ are not cycloalkyl, aryl, heteroaryl or heterocyclyl at the same time;
provided that R⁷, R⁸, R⁹ and R¹⁰ are not cycloalkyl, aryl, heteroaryl or heterocyclyl at the same time;
R^{a} and R^{b} are independently selected from H, alkyl, cycloalkyl and aryl;
R^{c} is selected from the optionally substituted group consisting of alkyl, alkoxy, alkoxyalkyl, amino, alkyl-C(O)—NR^{a}R^{b}, cycloalkyl, cycloalkylalkyl, —NR^{a}R^{b}, alkyl-NR^{a}R^{b}, —NH-alkyl-NR^{a}R^{b}, heteroaryl, heteroarylalkyl, heterocyclyl, heterocyclylalkyl, phenylalkylamino, —NH-heteroaryl and —NH-heterocyclyl
R^{d} is selected from the optionally substituted group consisting of —(CH₂)ₘ—NH₂, alkyl, alkoxy, aryl, heteroaryl, heterocyclyl and arylalkyl;
m is 1 or 2; and
n is 1,2, 3 or 4; or
Q is O; and
R¹ is selected from the optionally substituted group consisting of alkyl, —(CH₂)ₙC(O)—OCH₃, —(CH₂)ₙC(O)—O-alkyl, C(O)-phenyl, adamantanyl, benzo[1,3]dioxolyl, benzyl, cyclohexyl, cyclopentyl, diphenylmethyl. fluorenyl, indanyl, isoquinolinyl. diphenylmethyl, naphthyl, phenyl, piperidinyl, quinolinyl and thienyl; or
R¹ is A-B wherein
A is selected from the optionally substituted group consisting of methyl, ethyl, phenoxy and phenyl;
B is selected from the optionally substituted group consisting of benzyloxy, furanyl, phenoxy and phenyl;
X is selected from the group consisting of a bond, C(O) and C(O)NH;
R² is selected from the optionally substituted group consisting of benzimidazolyl, benzoxazolyl, benzyl, cyclohexyl, phenyl, piperidinyl, pyrazinyl, pyridazinyl, pyrimidinyl, pyrrolo[3,2-d]pyrimidinyl. quinazolinyl quinolinyl, quinoxalinyl and thieno[3,2-d]pyrimidinyl; or
R² is Y-Z wherein Y is attached to X and
Y is selected from the group consisting of naphthyl, phenyl, pyridazinyl, pyrimidinyl, tetrazolyl and 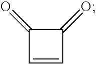
Z is selected from the optionally substituted group consisting of phenoxy, phenyl and piperazinyl;
R³ is H or isopropyl;
R⁴, R⁵, R⁷, R⁸ and R¹⁰ are H;
R⁶ is H, isopropyl or phenyl;
R⁹ is H, isopropyl or optionally substituted phenyl;
provided that X—R² is not H;
provided that the compound is not 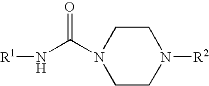
wherein
R¹ is phenyl substituted with CF₃, piperidinyl substituted with methyl, cyclohexyl substituted with one or more C(O)OH, methyl, CF₃, methoxy, F, Cl or H or 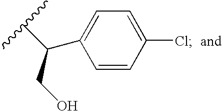
R² is benzyl, phenyl optionally substituted with one or more OH, Cl or methoxy, piperidinyl substituted with methyl, pyrimidinyl substituted with Cl or quinolinyl substituted with Cl;
provided that the compound is not 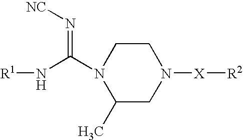
wherein
X is a bond;
R¹ is selected from the group consisting of methyl, ethyl, t-butyl, butyl, cyclohexyl, furanylmethyl, pyridinylmethyl, pyridinylethyl, optionally substituted phenyl, optionally substituted benzyl, optionally substituted phenylethyl, optionally substituted phenylpropyl, 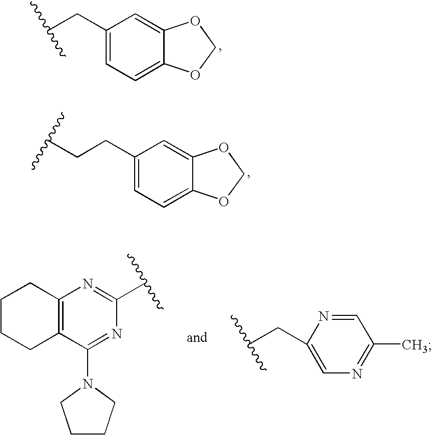
wherein the substituents are selected from the group consisting of Cl, Br, F, methyl, propyl, isobutyl, butyl, t-butyl, OCH3, isopropoxy, O-t-butyl, cyclohexyl, CF₃, SCH₃, SO₂CH₃ and CH₂C(CF₃)₃; and
R² is selected from the group consisting of phenylethyl, 1,2,3-triazolyl, pyridinyl substituted with Cl, quinolinyl substituted with Cl, 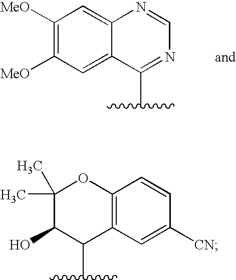
provided that the compound is not 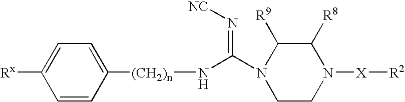
wherein
X is a bond;
R^{x} is selected the group consisting of from t-butyl, isobutyl, sec-butyl, t-butoxy, isopropyl, CF₃, ethyl, OCF₃, halo, n-butyl and n-propyl;
R⁸ and R⁹ are independently H or methyl;
R² is selected from the group consisting of 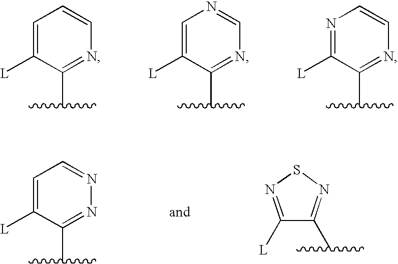
wherein L is Cl, methyl, CF₃, OH, NO₂, CN, Br, I or F; and
n is 0, 2 or 3.

2. The compound of claim 1 wherein Q is O.

3. The compound of claim 2 wherein X is C(O).

4. The compound of claim 3 wherein
R¹ is selected from the optionally substituted group consisting of benzyl, naphthyl and phenyl;
wherein the benzyl is substituted with methyl or Cl and the phenyl is optionally substituted with one or more methyls;
R² is benzyl substituted with two OCH₃; and
R⁶ is H.

5. The compound of claim 2 wherein X is a bond.

6. The compound of claim 5 wherein
R¹ is selected from the optionally substituted group consisting of naphthyl, phenyl and quinolinyl;
R² is selected from the optionally substituted group consisting of pyrazinyl, pyridazinyl, pyrimidinyl, pyrrolo[3,2-d]pyrimidinyl and thieno[3,2-d]pyrimidinyl; or
R² is Y-Z wherein Y is pyridazinyl and Z is phenyl.

7. The compound of claim 2 wherein X is C(O)NH wherein the C(O) is attached to the nitrogen of the piperazine.

8. The compound of claim 7 wherein
R¹ is selected from the optionally substituted group consisting of naphthyl and benzyl;
R² is 4-chlorophenyl;
R³, R⁴, R⁵, R⁶, R⁷, R⁸ and R¹⁰ are H; and
R⁹ is isopropyl.

9. The compound of claim 8 wherein R¹ is benzyl substituted with methyl or Cl.

10. The compound of claim 1 wherein Q is S.

11. The compound of claim 10 wherein X is a bond.

12. The compound of claim 11 wherein
R¹ is quinolinyl;
R² is Y-Z wherein
Y is pyridazinyl; and
Z is benzo[b]thiophenyl substituted with methyl;
R³ is isopropyl; and
R⁴, R⁵, R⁶, R⁷, R⁸, R⁹, R¹⁰ are H.

13. The compound of claim 12 wherein X is C(O)—NH wherein the C(O) is attached to the nitrogen of the piperazine.

14. The compound of claim 13 wherein R¹ is 2,3-dihydrobenzofuranyl, quinolinyl or phenyl wherein
the quinolinyl is optionally substituted with methyl and the phenyl is substituted with —C(O)OCH₃ or —S(O)₂CH₃;
R² is phenyl substituted with Cl, F or CF₃;
R³ is H or isopropyl;
R⁴, R⁵, R⁶, R⁷, R⁸ and R¹⁰ are H; and
R⁹ is H or isopropyl.

15. The compound of claim 1 wherein Q is N—CN.

16. The compound of claim 15 wherein X is a bond.

17. The compound of claim 16 wherein
R¹ is selected from the optionally substituted group consisting of dihydrobenzofuranyl, indazolyl, isoquinolinyl, phenyl, quinolinyl and tetrahydroquinolinyl; or
R¹ is A-B wherein
A is unsubstituted isoquinolinyl or phenyl substituted with methyl; and
B is selected from the group consisting of —C(O)—OCH₂-phenyl, —C(O)—CH₃ and —C(O)—N(CH₃)₂;
R² is selected from the optionally substituted group consisting of benzimidazolyl, benzoxazolyl, imidazo[1,2-b]pyridazinyl, pyrazinyl, pyridazinyl, pyridinyl, pyrimidinyl, pyrrolo[3,2-d]pyrimidinyl, quinazolinyl, quinoxalinyl, thiazolyl, thieno[2,3-d]pyrimidinyl, thieno[3,2-d]pyrimidinyl and thienyl; or
R² is Y-Z wherein
Y is selected from the optionally substituted group consisting of pyrazinyl, pyridazinyl, pyrimidinyl and 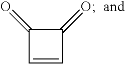
Z is selected from the optionally substituted group consisting of C(O)-morpholinyl, C(O)-piperazinyl, C(O)-piperidinyl, C(O)-pyrrolidinyl, —C(O)—NH-isoxazolyl, —C(O)—NH-phenyl, —C(O)—NH-pyridinyl, —C(O)—NH-thiazolyl, —C(O)NHCH₂CH₂OH, NHCH₂CH₂-morpholino, phenyl, piperazinyl, pyrazolyl and pyridinyl; and
R⁹ is isopropyl.

18. The compound of claim 54 wherein X is C(O)NH wherein the C(O) is attached to the piperazine.

19. The compound of claim 18
R¹ is phenyl substituted with methyl or R¹ is unsubstituted quinolinyl;
R² is unsubstituted dihydrobenzo[1,4]dioxinyl, unsubstituted thienyl or phenyl substituted with one or more CN, Cl, F, SO₂CH₃ or OCH₃; or
R² is Y-Z wherein Y is ethyl and Z is phenyl substituted with one or more C(O)CH₃ or OCH₃, or Z is unsubstituted CH₂—NH—C(O)—OCH₂-phenyl;
R³ is isopropyl; and
R⁷ is phenyl.

20. The compound of claim 15 wherein X is C(O).

21. The compound of claim 20 wherein
R² is selected from the optionally substituted group consisting of adamantanyl, benzyl, indolyl, phenyl, pyrazinyl, pyrazolyl, pyrrolyl, thiazolyl and thienyl; or
R² is Y-Z wherein
Y is selected from the optionally substituted group consisting of methyl, ethyl, propyl, pyridinyl and thienyl; and
Z is selected from the optionally substituted group consisting of benzotriazolyl, furanyl, isoxazolyl, morpholinyl, oxazolyl, phenyl, pyrazolyl, pyridinyl and thienyl;
R⁷ is isopropyl or phenyl; and
R⁹ is H, isopropyl or phenyl.

22. The compound of claim 15 wherein X is S(O)₂.

23. The compound of claim 22 wherein
R¹ is phenyl substituted with methyl or NH—C(O)CH₃;
R² is unsubstituted benzo[1,2,5]oxadioazolyl, unsubstituted benzo[1,2,5]thiadiazolyl, benzoxazolyl substituted with oxo, phenyl substituted with one or more methyl, F, CN, Cl, or OCH₃ or thienyl optionally substituted with methyl; and
R⁷ is phenyl.
